(19) United States
(12) Patent Application Publication
(10) Pub. No.: US 2008/0131451 A1
(43) Pub. Date: Jun. 5, 2008

(54) Epitope escape mutations

(76) Inventors: Giancarlo Tanzi, Hopkinton, MA (US); Shaun Geer, Los Alamos, NM (US)

Correspondence Address: Ingrid A. Beattie, Ph.D., J.D.;Mintz Levin Cohn Ferris Glovsky & Popeo, P.C., One Financial Center, Boston, MA, 02111, US

(21) Appl. No.: 11/076,381

(22) Filed: Mar. 7, 2005

Related U.S. Application Data
(60) Provisional application No. 60/550,766, filed on Mar. 5, 2004.

(51) Int. Cl. A61K 39/00; C12Q 1/70

(52) U.S. Cl. 424/188.1; 435/5

(57) ABSTRACT

The present invention provides methods for predicting epitope escape mutations arising during the course of HIV disease using an algorithm that predicts the difficulty with which and the manner in which HIV may escape from a CTL response. Thus, the invention is useful for the development of effective therapeutic or prophylactic vaccines against highly variable pathogens, such as HIV, Hepatitis C, and Influenza virus.

### RELATED APPLICATION

This application claims priority to U.S. Ser. No. 60/550,766, filed Mar. 5, 2004. The contents of this application are incorporated herein by reference in its entirety.

### STATEMENT AS TO FEDERALLY SPONSORED RESEARCH

This invention was made with U.S. government support under NIH grant, number NIH NS 26084. The government has certain rights in the invention.

### FIELD OF THE INVENTION

The present application relates to the field of vaccines.

### BACKGROUND OF THE INVENTION

Given that viral load in patients is largely controlled by cytotoxic T Lymphocytes (CTL), many vaccines are designed to elicit cellular immune responses against pathogens. This approach, however, is limited in Human Immunodeficiency Virus (HIV) vaccines. Current HIV vaccines employ immunizing epitopes that correspond to the most common and most potent CTL responses in infected patients. Over time however, genetic mutations within the HIV genome allows the virus to evade the CTL-mediated immune response. These ‘escape mutations’ often occur in or adjacent to HIV epitopes that are typically recognized and presented to the immune system. These mutations are thought to have an important role in the loss of immune control of HIV seen in most patients.

Thus, vaccines having the ability to generate a strong immune response that do not allow for escape mutations are needed.

### SUMMARY OF THE INVENTION

The present invention features immunogenic compositions useful for the treatment and prevention of pathogenic infections, such as a viral infection (e.g., HIV-1), bacterial infection, fungal infection, and parasitic infection.

In one aspect, the invention provides immunogenic compositions that contain an epitope of a pathogenic protein (e.g., HIV-1 protein such as Pol, Vif, Tat, Rev, Vpu, Env, Vpr, or Nef) having an amino acid sequence with an entropy score that is less than the average entropy score of said pathogenic protein. The epitope is preferably in a conserved region of a structural protein of HIV-1 such as Gag and/or Pol. A conserved region is a fragment of a protein in which two or more consecutive residues have an entropy score that is greater than the average entropy score of residues of the full length of the protein. For example, the entropy score or conservation score of the pathogenic protein may be 0.900, 0.850, 0.800, 0.750, 0.700, 0.650, or less. An entropy score is determined by ΣPₐₐlogPₐₐ, where Pₐₐ is the proportion of each amino acid in the respective position. Thus, an entropy score is a numerical value that reflects the amount of amino acid variability at a given position in a patient-derived protein isolate. Accordingly, a high level of variability (and corresponding low entropy score) indicates a position that is subject to epitope escape mutations. Desirably, the entropy score is less than an average of the entropy scores of each residue position in the target protein.

Alternatively, the invention features an immunogenic composition containing two, three, four, five, or more peptides that are epitopes of a pathogenic protein (e.g., HIV-1 protein) and that are substantially identical to one another. Thus, the epitopes may share the same amino acid sequence but differ at least at one amino acid position, which is desirably at the escape mutation site or at the site with low entropy score (e.g., less than the average entropy score of the pathogenic protein). For example, the peptides may be epitopes of an HIV-1 protein, all of which have the same amino acid sequence with the exception of the presence of at least one amino acid position having an entropy score less than the average entropy score of the HIV-1 protein (see TABLES 10-12). For example, one peptide contains an HIV consensus sequence (e.g., STTVKAACWW (SEQ ID NO: 1), IVLPEKDSW (SEQ ID NO: 2), AIFQSSMTK (SEQ ID NO: 3), SPAIFQSSM (SEQ ID NO: 4), ACQGVGGPGHK (SEQ ID NO: 5) or GSEELRSLY (SEQ ID NO: 6)) and a second peptide that differs from the first peptide by a nonidentical amino acid at the amino acid position with low entropy score.

For example, the composition may contain various epitopes of the Pol protein having the amino acid sequences STTVKAACWW (SEQ ID NO: 1), IVLPEKDSW (SEQ ID NO: 2), AIFQSSMTK (SEQ ID NO: 3), or SPAIFQSSM (SEQ ID NO: 4), for example. The composition of the invention may therefore include a mixture of peptides containing the amino acid sequence STTVKAACWW (SEQ ID NO: 1) in addition to peptides having the amino acid sequence SNTVKAACWW (SEQ ID NO: 7), SATVKAACWW (SEQ ID NO: 8), STAVKAACWW (SEQ ID NO: 9), STSVKAACWW (SEQ ID NO: 10), or combinations thereof. Alternatively, the composition may include peptides having the amino acid sequence IVLPEKDSW (SEQ ID NO: 2) and peptides having the amino acid sequence IMLPEKDSW (SEQ ID NO: 11), ITLPEKDSW (SEQ ID NO: 12), IKLPEKDSW (SEQ ID NO: 13), or combinations thereof. The composition may also contain a peptide having the amino acid sequence AIFQSSMTK (SEQ ID NO: 3) and peptides having the amino acid sequence AIFQSSMTR(SEQ ID NO: 14), AIFQSSMTT (SEQ ID NO: 15), AIFQSSMTI (SEQ ID NO: 16), or combinations thereof. The composition may also contain a peptide having the amino acid sequence SPAIFQSSM (SEQ ID NO: 4) and peptides having the amino acid sequence SPAIFQCSM (SEQ ID NO: 17), SPAIFQYSM (SEQ ID NO: 18), or combinations thereof.

Optionally, the immunogenic composition contains epitopes of the Gag protein having the amino acid sequences ACQGVGGPGHK (SEQ ID NO: 5) or GSEELRSLY (SEQ ID NO: 6), for example. For example, the composition may contain a mixture of peptides having the amino acid sequence ACQGVGGPGHK (SEQ ID NO: 5) and ACQGVGGPSHK (SEQ ID NO: 19). Alternatively, the composition contains a mixture of peptides having the amino acid sequence GSEELRSLY (SEQ ID NO: 6) and GSEELRSLF (SEQ ID NO: 20).

The polypeptides and nucleic acids described herein may also be formulated as vaccines or may be employed for vaccine development. The polypeptides and nucleic acids may be derived from a microorganism, such as a pathogenic bacterium, virus, or fungus and may be substantially pure.

The present invention further features a method of determining an escape mutation site within a protein epitope (e.g., a protein of a highly variable pathogenic organism) that includes the steps of: (1) determining a first entropy score for a first amino acid position of a target protein and (2) comparing the first score to a second score for a second amino acid position of the same target protein or to an average of scores of 2 or more positions of the target protein. For example, the first entropy score may be compared to an average of the scores of all the positions of the target protein. An escape mutation is determined by identifying a low entropy score (e.g., less than the numerical average entropy score).

The invention also features a method of determining the composition of an escape mutation resistant-vaccine. An escape mutation resistant vaccine is a vaccine that contains at least two immunogens having a substantially identical amino acid sequence but differing at least at one escape mutation site. Desirably, the vaccine contains 2, 3, 4, 5, 6, 7, 8, 9, or 10 or more substantially identical peptides, differing by at least one amino acid substitution at the site of an escape mutation. The composition of such a vaccine may be determined by (1) providing an entropy score profile of a target protein; (2) selecting an amino acid sequence within the target protein that contains an amino acid site corresponding to a low entropy score; (3) determining the identity of amino acid variants at the amino acid site with low entropy score from a patient-derived index of amino acid sequences of the target protein; and (4) selecting one, two, three, or more peptides, each containing the amino acid sequence with the amino acid site with low entropy score and each containing a different amino acid variant at the amino acid site with low entropy score. Alternatively, step (4) involves selecting at least two substantially identical peptides, containing the same amino acid sequence having the amino acid position with low entropy score but having a different amino acid at such position.

Desirably, the composition contains a mixture of at least 2, 3, 4, 5, or more peptides containing an amino acid substitution at the amino acid site that was found to correspond to a low entropy score. Data pertaining to amino acid sequence variation of viral proteins of patient-derived viral isolates are described herein and summarized and available in database format. The amino acids substitutions are selected from amino acids previously observed to be present in escape mutation viral isolates taken from human patients at various stages of disease progression. Such amino acid variation information may be obtained from public databases.

According to the present invention, an “epitope escape mutation” is a change in an epitope of a pathogen resulting from a genetic mutation that reduces the ability of the immune response to recognize the pathogen. The escape mutation may render the pathogen less susceptible to killing, elimination, or neutralization by the immune response elicited by the primary infectious agent. Accordingly, the ability of the immune system to recognize the epitope with an escape mutation may be reduced by at least 5%, 10%, 20%, 30%, 40%, 50%, 60%, 70%, 80%, 90%, 95%, or even 100% relative to a control epitope not having the escape mutation. For example, epitope escape mutations in HIV proteins lead to a reduced CTL response to HIV-infected cells.

A vaccine, as used herein, is a preparation including materials that evoke a desired biological response, e.g., an immune response, in combination with a suitable carrier. The vaccine may be administered by any standard route of administration including, for example, parenteral, subcutaneous, intradermal, and intramuscular administration.

By a “substantially pure polypeptide” is meant a polypeptide which is separated from those components (proteins and other naturally-occurring organic molecules) which naturally accompany it. A polypeptide is substantially pure when it constitutes at least 60%, by weight, of the protein in the preparation. Preferably, the protein in the preparation is at least 75%, more preferably at least 90%, and most preferably at least 99%, by weight, of the desired peptide. A substantially pure polypeptide is obtained, e.g., by extraction from a natural source; by expression of a recombinant nucleic acid; or by chemically synthesizing the protein. Purity can be measured by any appropriate method, e.g., column chromatography, polyacrylamide gel electrophoresis, or HPLC analysis. A protein is substantially free of naturally associated components when it is separated from those contaminants which accompany it in its natural state. Thus, a protein which is chemically synthesized or produced in a cellular system different from the cell from which it naturally originates will be substantially free from its naturally associated components. Accordingly, substantially pure polypeptides include recombinant polypeptides derived from a eukaryote but produced in E. coli or another prokaryote, or in a eukaryote other than that from which the polypeptide was originally derived.

The peptides to be used in vaccines are typically at least 8 residues in length. For example, the peptides range in length from 10-500 amino acids. Peptides, which are 10, 20, 30, 40, or 50 amino acids in length are preferred. For example, the peptide is 9 to 15 amino acids in length.

By “substantially identical” is meant a polypeptide or nucleic acid exhibiting at least 75%, but preferably 85%, more preferably 90%, most preferably 95%, or even 99% identity to a reference amino acid or nucleic acid sequence. For polypeptides, the length of comparison sequences is generally at least 5 amino acids, 8 amino acids, 10 amino acids, 20 amino acids, preferably at least 30 amino acids, more preferably at least 40 amino acids, and most preferably 50 amino acids. For nucleic acids, the length of comparison sequences will generally be at least 20 nucleotides, 40 nucleotides, 60 nucleotides, preferably at least 90 nucleotides, and more preferably at least 120 nucleotides.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65 C, or a buffer containing 48% formamide, 4.8×SSC, 0.2 M Tris-Cl, pH 7.6, 1×Denhardt's solution, 10% dextran sulfate, and 0.1% SDS, at a temperature of 42 C. Other conditions for high stringency hybridization, such as for PCR, Northern, Southern, or in situ hybridization, DNA sequencing, etc., are well-known by those skilled in the art of molecular biology. See, e.g., F. Ausubel et al., Current Protocols in Molecular Biology, John Wiley & Sons, New York, N.Y., 1998, hereby incorporated by reference.

Other embodiments and features of the invention will be apparent from the following description thereof, and from the claims.

### BRIEF DESCRIPTIONS OF THE FIGURES

FIG. 1 is a series of graphs showing the viral loads and CD4+ T cell counts in four subjects identified during acute HIV-1 infection and followed longitudinally. Arrows (denote time points from which viral sequences were derived. Shaded regions denote time under highly active antiretroviral therapy (HAART).

FIGS. 2A-B are schematics showing sites of sequence variation and associated CD8 T cell responses. Longitudinal whole genome sequencing, excluding the env gene, was conducted using population sequencing FIG. 2A shows mutations accumulating over time in subject AC-38 over 4 years of follow-up. All mutations except #15 persisted once they were detected. FIG. 2B shows mutations observed to develop in each of four study subjects over 0.3-5.3 years of follow-up. (o) denotes mutations arising over the course of follow-up. (•) denotes mutations associated with detectable IFN-γ Elispot CD8+ T cell responses.

FIG. 2C is a pie chart showing the percentage of mutations associated with CD8+ T cell responses and reversion. In cases where associations with HLA alleles were detected are indicated separately. Numbers within each region of the pie graph indicate the number of mutations, out of a total of 98 mutations tracked, associated with each selective pressure.

FIGS. 3A-3B are a series of bar graphs showing that escape mutations in Gag and Pol occur at highly variable residues in the population. The degree of variability of residues in Gag and Pol was assessed by plotting Shannon entropy values for each residue. Below each epitope is indicated the residue observed to arise during sequence evolution in a subject. Solid bars denote escape mutations corresponding to the most polymorphic residue in each epitope, hatched bars denote escaping residues not corresponding to the most polymorphic residue in each epitope. CD8-targeted regions in which mutations were observed include B57 epitopes (FIG. 3A) and published epitopes (Pub), optimal epitopes in study subjects (i.e. AC-38), and unmapped responses in study subjects (unmapped) (FIG. 3B).

FIGS. 4A and 4B are a series of bar graphs showing the shared polymorphic residues in Gag and Pol in Clade B and C. The degree of variability of clade C sequences are plotted against clade B Gag and Pol epitopes shown in FIG. 3. Black bars denote clade B sequences while grey bars denote clade C sequences. (*) indicates highly polymorphic residues shown to mutate in FIG. 3 which are shared across clade B and clade C. FIG. 4A represent B57 epitopes and FIG. 4B represents published epitopes (Pub), optimal epitopes in study subjects (i.e. AC-38), and unmapped responses in study subjects (unmapped).

FIGS. 5A-5D are schematics showing the alignment of HIV-1 sequences in four study subjects. Whole genome sequences, excluding Env, from early and late time points are aligned. Dark shaded regions denote mutations within optimal CD8 epitopes in which CD8+ T cell responses were detected. Light shaded regions denote mutations associated with novel, unmapped CD8+ T cell responses. Individually shaded residues denote mutations not associated with detectable CD8+ T cell responses. Mutations with a) and b) suffix's indicate mutations resulting in substitutions within overlapping reading frames of different proteins. Time points for each subject include: AC-38 (day 64 vs. day 1510), AC-59 (day 41 vs. day 111), AC-04 (day 0 vs. day 1650), AC-16 (day 0 vs. day 1934). Underlined regions indicate insertions in comparison to the clade B consensus sequence.

FIG. 6 is a series of bar graphs showing escape mutations in accessory and regulatory proteins. The degree of variability of residues in accessory and regulatory proteins was assessed by plotting Shannon entropy values for each residue. Below each epitope is indicated the residue observed to arise during sequence evolution. Solid bars denote escape mutations corresponding to the most polymorphic residue in each epitope, hatched bars denote escaping residues not corresponding to the most polymorphic residue in each epitope. CD8-targeted regions in which mutations were observed were derived from published epitopes (Pub), optimal epitopes in study subjects (i.e. AC-38), and unmapped responses in study subjects (unmapped).

FIGS. 7A-7B are tables showing the mutations associated with HLA alleles expressed by the current host and transmitted HLA-associated mutations reverting to consensus.

FIG. 8 is a table showing stereotypic CD8 escape mutations observed in chronic Gag and Pol HLA-B57 epitopes.

FIG. 9 is a table showing regions of HIV-1 exhibiting sequence variation and associated CD8+ T cell responses.

FIG. 10 is a table showing conserved regions associated with CD8+ T cell responses.

### DETAILED DESCRIPTION

The present invention allows for the prediction of HIV CTL epitope escape mutations arising during the course of HIV disease using an algorithm that predicts the difficulty with which and the manner in which HIV may escape from a CTL response. This information, in turn, allows the identification of peptide sequences for the development of effective therapeutic or prophylactic vaccines against highly variable pathogens, such as HIV, Hepatitis C, and Influenza virus. In this regard, the present invention facilitates the choice of vaccine epitopes and the choice of vaccine escape variants to be formulated within a vaccine.

The relationship between genome-wide viral evolution and adaptive CD8+ T cell responses following acute HIV-1 infection was assessed. Our results show that between 48-60% of evolving mutations in non-envelope proteins were associated with detectable virus-specific CD8+ T cell responses in 4 clade B virus-infected patients studied longitudinally up to 5 years after acute infection. Together with additional mutations consistent with reversion of transmitted CD8+ T cell-driven mutations, nearly two thirds of all mutations were attributable to CD8+ T cell selective pressures. Expanded studies in persons with chronic disease indicated that selection pressure frequently resulted in identical amino acid substitutions in mutating epitopes, and mutating residues correlated with highly polymorphic sites in both clade B and C viruses.

The striking reproducibility of many escape mutations, their predominance at highly polymorphic sites across different HIV-1 clades, and the observed reversion of HLA-associated polymorphisms, together suggest that biochemical constraints limit which residues within a targeted HIV-1 CD8 epitope are likely to mutate in response to CD8+ T cell selective pressures. Some mutations may therefore be induced by immune-mediated selective pressures have an impact upon viral replication capacity, especially mutations developing within more conserved regions of the virus. Thus, vaccines may be designed to target highly conserved regions of HIV-1 and to exploit the limitations of HIV-1 evolution.

Prediction of Amino Acid Variability to Identify Location of Escape Mutations

The ability of HIV to mutate is in large part a result of the degree of structural flexibility of HIV proteins. As a proxy for structural flexibility, the degree of amino acid variability at each position of the HIV proteome was determined. The lack of variability at a given amino acid position indicates structural constraint. For example, known structurally important regions of HIV Gag exhibited very low amino acid variability scores. The determination of HIV amino acid variability was possible because of the large degree of variability observed between HIV isolates and the large number of HIV sequences available in the public database.

HIV amino acid variability was determined by calculating amino acid entropy values (e.g., Shannon entropy scores) for each amino acid of HIV as described, for example in Yusim et al. 2002, J. Virol. 76:8757 or www.ebi.ac.uk/thorntonsrv/databases/valdarprograms/scorecons_server-help.html. Entropy is a measure of the amino acid variability at a given position that takes into account both the number of possible amino acids allowed and their frequency. Entropy in each amino acid position was determined employing the calculation: −ΣPₐₐlogPₐₐ, where Pₐₐ is the proportion of each amino acid in the respective position. The numerical value calculated reflects both the number of amino acids found at a given position of an HIV protein and also the frequency of each amino acid. The consensus sequence, i.e. the base sequence to which the database variations were compared, of each HIV protein examined were obtained from GenBank (the full length genome for HIV HXB2, GenBank accession number K03455; www.hiv.lanl.gov/content/hiv-db/REVIEWS/HXB2.html).

Shannon entropy scores were calculated for each position of the protein alignment. To determine the degree of amino acid entropy of translated HIV sequences found in the database, between 43 and 119 (depending on the protein) nucleic acid alignments of HIV-1 were retrieved from public Los Alamos National Laboratory HIV Sequence database. These sequences were selected on the basis of two criterions: 1) the sequence was derived from a B clade HIV viral isolate and 2) the sequence had a unique patient identifier indicating that the sequence was unique. The nucleotide sequences were aligned and insertions were removed. The sequence was translated into an amino acid sequence, and Shannon's entropy score was determined for each amino acid position in HIV proteins.

Once amino acid entropy was determined, studies were undertaken to determine if a relationship exists between HIV amino acid escape and the HIV amino acid entropy score. To test this relationship, CTL sequence escape data obtained from longitudinal studies was used. Data containing 15 epitopes and 16 escape mutations was analyzed. Specifically, CTL responses from patients were mapped early in infection and during subsequent chronic infection. Many of the early responses were lost as a result of HIV acquiring escape mutations. Examination of the entropy score of escaped amino acids demonstrated a very strong relationship between HIV entropy and amino acid escape. Specifically, amino acids with higher entropy, i.e., lower Shannon entropy scores, were far more likely to give rise to escape mutations than were amino acids that were invariable in the database. Based on the entropy of positions within a CTL epitope, the probability that particular residues will mutate may be estimated. In addition, the analysis determined that, in almost all cases, escaped residues mutate to amino acids already represented in the database. Thus, using a known CTL epitope sequence, the probability that an amino acid will escape and the amino acid variant that will predominate may be predicted.

Overall, the present invention addresses several issues that limit the development of a CTL based HIV vaccine. In this regard, the ability to predict HIV CTL epitope(s) that, due to protein invariability, are likely to escape immune detection, permits the identification of effective epitopes for CTL targeted HIV vaccine design. In this regard, the relationship between entropy and escape allows the prediction of sites where vaccine escape mutations arise, the specific amino acids most likely to escape, and the amino acids most likely to arise in escape variants. Based on such information, epitopes which are most effective in viral control are determined and vaccines may be designed accordingly. For example, a vaccine may be designed to include a peptide that contains an amino acid sequence corresponding to a linear CTL consensus sequence in which the site corresponding to a low score contains an amino acid selected from the group of residues observed in a database of patient-derived sequences in that particular position. Individuals are then immunized with the most common CTL consensus sequence as well as probable CTL escape variants.

TABLE 10 shows epitope average data, demonstrating that the average epitope conservation value of a stable epitope is higher than that of a non-fixed escape epitope. TABLE 11 shows residue conservation data, demonstrating that the epitope residues that remained fixed have on average higher conservation values than those that escape. The difference was quite large (0.19) with p value<0.005. The statistics program at http://calculators.stat.ucla.edu/ was employed for this analysis. TABLE 12 shows residue variation data, demonstrating that the amino acids to which the residues change when epitopes escape is predicted. The amino acid changes that were found within a patient and also the various amino acids found at that position in the database are shown. One to five amino acids (e.g., one, two, or three) are typically observed at a given escaping position. Furthermore, when an amino acid escapes, it usually changes to an amino acid that is well represented in the database indicating that a given residue only tolerates a small number of amino acids and that in the event that an amino acid position does mutate, the amino acid at such position is usually another amino acid predictable. Accordingly, a vaccine is designed by incorporating the consensus epitope and one or more of the predicted escape variants. Such vaccines designs are particularly useful in view of the concept known as “original antigenic sin,” in which the body is unable to mount a new immune response to an epitope that mutates (see, for example, Klenerman et al., Nature 394:482-5, 1998) and which may contribute to the ability of HIV to escape an epitope in the absence of the immune system mounting an immune response to the mutated epitope. Thus, if a mammal is able to mount immune responses to an epitope and common escape variants at the time of vaccination, HIV is less likely to effectively escape the immune response.

Thus, a vaccine may be designed to contain two, three, four, five, or more peptides that are epitopes of a pathogenic protein (e.g., HIV-1 protein) and that are substantially identical to one another. The epitopes may share the same amino acid sequence but differ at least at one amino acid position, which is desirably the escape mutation site or the site with low entropy score (e.g., less than the average entropy score of the pathogenic protein). For example, the peptides may be epitopes of an HIV-1 protein, all of which have the same amino acid sequence with at least one amino acid position having an entropy score less than the average entropy score of the HIV-1 protein (see TABLES 10-12). The peptides, however, have a different amino acid at the amino acid position with low entropy score.

Vaccine Administration

Vaccine compositions (peptides or combinations of peptide variants) are administered to a patient in the form of a peptide solution in a pharmaceutically acceptable carrier using standard methods known in the art. Standard methods for intracellular delivery of peptides can be used, e.g. packaged in liposomes. It is expected that an intravenous dosage of approximately 1 to 100 μmoles of the polypeptide of the invention would be administered per kg of body weight per day. The compositions of the invention are useful for parenteral administration, such as intravenous, subcutaneous, intramuscular, and intraperitoneal. Vaccines are also administered orally or nasally. For example, a unit dose of the peptide ranges from 0.1 to 100 mg, which may be administered, one time or repeatedly, to a patient. Optionally, the HIV peptide formulated in the vaccine contains an amino acid sequence with an amino acid site associated with a low entropy score. An HIV peptide may be administered alone or may be administered with one, two, three, or more epitope peptides (simultaneously or sequentially). If desired, an HIV peptide with an amino acid site associated with low entropy score is administered with one, two, three, or more peptides with an amino acid substitution at the amino acid site associated with low entropy containing an amino acid site are administered together.

Peptides are recombinantly produced or synthetically made using known methods. Peptide solutions are optionally lyophilized or granulated with a vehicle such as sugar. When the compositions are administered by injection, they are dissolved in distilled water or another pharmaceutically acceptable excipient prior to the injection. The peptides are optionally mixed with an agents which enhance the immunogenicity of the peptides; such agents include BCG bacterial cells, ISCOM (Immunostimulating complex), QS-21, aluminum hydroxide (alum), and KLH (Keyhole Limpet Hemocyanin).

The peptides are used to induce anti-HIV CTL activity in vivo or ex vivo. The peptides may be administered directly to an individual. Alternatively, lymphocytes (e.g., derived from peripheral blood mononuclear cells) are removed from an individual, cultured with one or more peptides, and returned to the individual. For example, 0.01 to 1 mg of the peptide is added to 10⁷ to 10⁹ peripheral blood lymphocytes originated from a patient, then the cells are cultivated for several hours to one day and thereafter they are intravenously administered to the patient. Optionally, cells are continuously cultured in vitro in a culture medium to which recombinant interleukin 2 and 1 μg/ml of the peptide has been added. The cells are cultured over several weeks to induce CTL. Activated CTL are then intravenously injected into the patient.

If desired, DNA encoding a peptide epitope is incorporated into a viral vector for intracellular expression. The DNA is administered in a pharmaceutically acceptable carrier. The therapeutic composition may also include a gene delivery system such as a viral vector system. Pharmaceutically acceptable carriers are biologically compatible vehicles which are suitable for administration to an animal e.g., physiological saline. A therapeutically effective amount is an amount of the nucleic acid of the invention which is capable of producing a medically desirable result in a treated animal. As is well known in the medical arts, dosage for any given patient depends upon many factors, including the patient's size, body surface area, age, the particular compound to be administered, sex, time and route of administration, general health, and other drugs being administered concurrently. For example, nucleic acids are administered intravenously at a dose of approximately 10⁶ to 10²² copies of the nucleic acid molecule. Determination of optimal dosage is well within the abilities of a pharmacologist of ordinary skill.

### EXAMPLE 1

### Immune Response Driven-Mutation Analysis

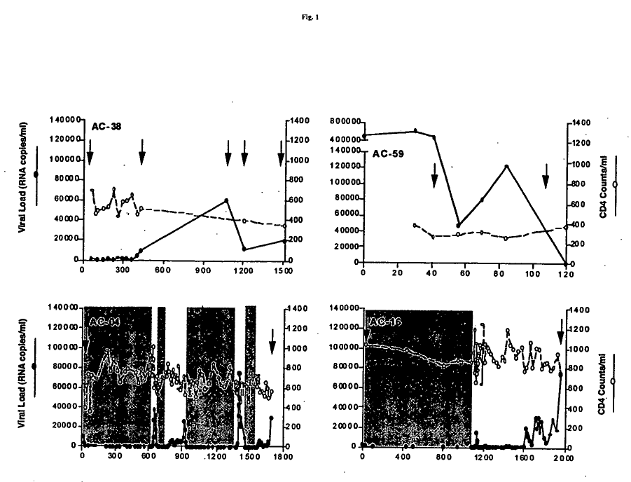

To determine the relationship between immune selection pressure and intra-host viral evolution, we performed whole genome sequencing together with assessment of CD8+ T cell responses in four HIV-1-infected subjects identified during acute infection, two of whom were transiently treated with antiretroviral drugs (see FIG. 1). For this analysis, we excluded the envelope gene which is known to be under dominant humoral immune selection pressure. Population sequencing of circulating plasma virus performed at the time of acute infection showed a homogeneous virus population in each person, but with the infecting viruses differing from one another by 8.7%-10.8%.

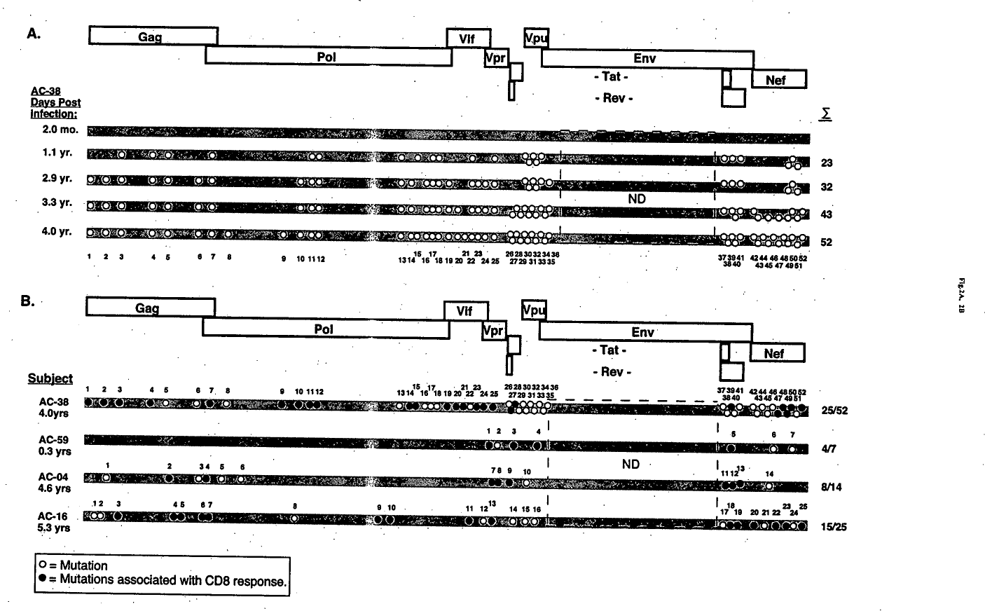

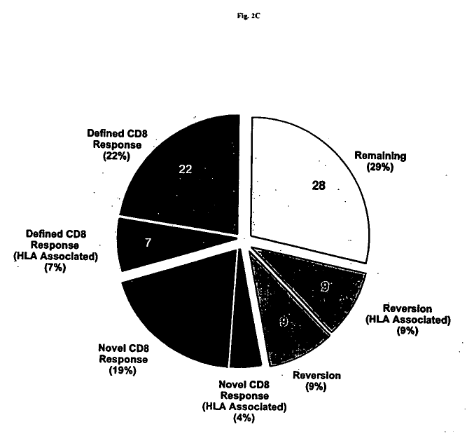

Longitudinal viral sequence data indicated the progressive accumulation of mutations that occurred in a step wise fashion. Representative data are presented in FIG. 2A for subject AC-38, in whom a total of 52 mutations arose sequentially over the 4 years of follow-up. Nearly half of these mutations occurred in the first year after infection before viral loads rose above 5,000 copies/ml; all but one mutation remained stable subsequently. Similar analyses were performed in all 4 persons, ranging from 0.3 to 5.3 years of follow up and showed evolution of 7 to 52 mutations (FIG. 2B).

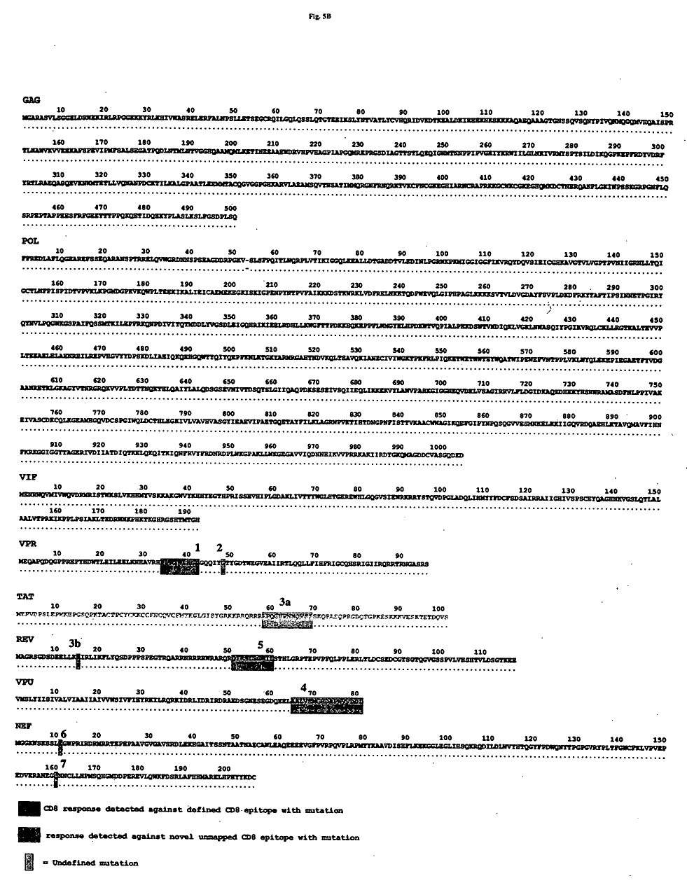

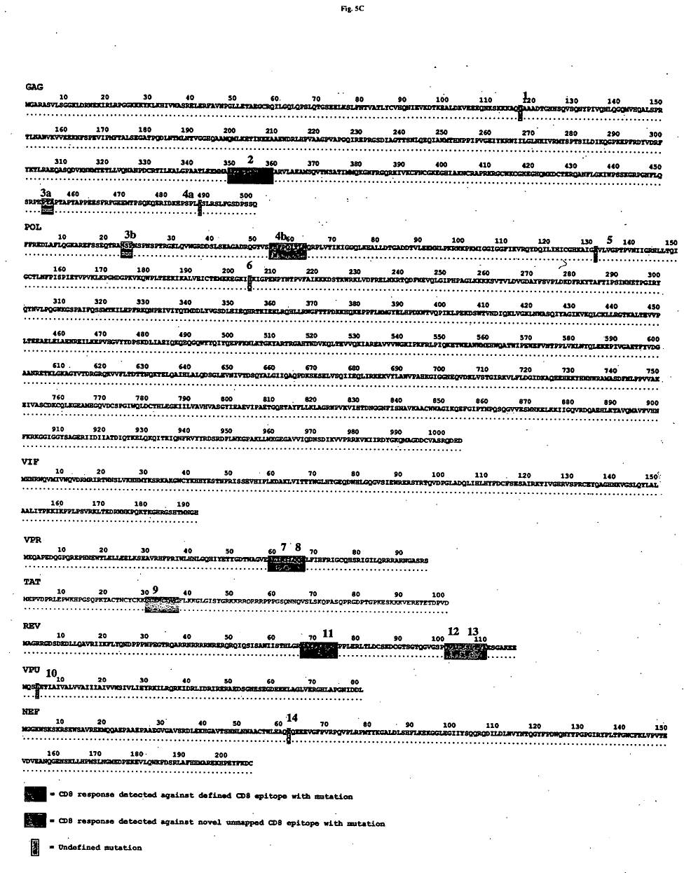

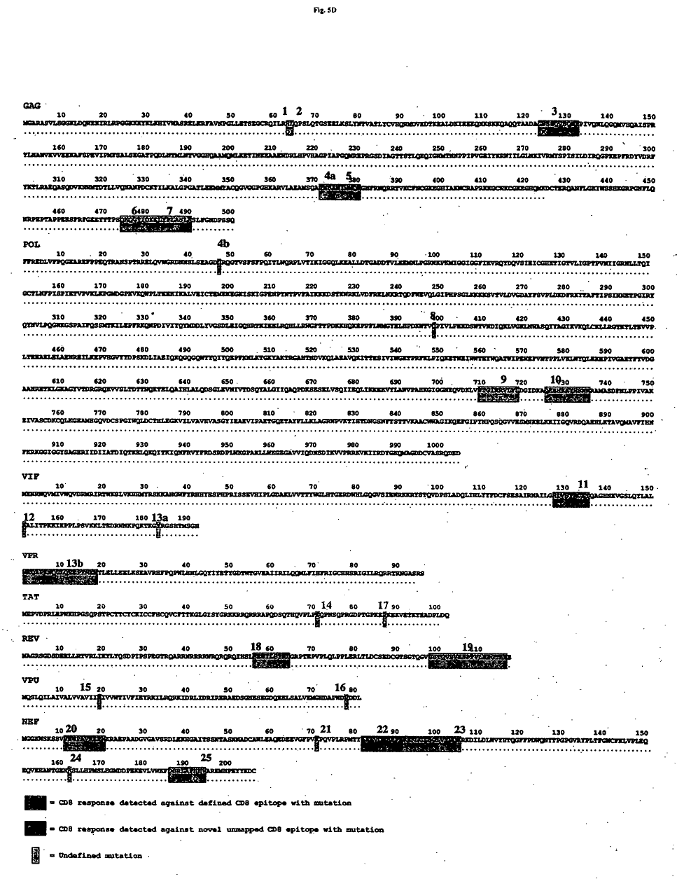

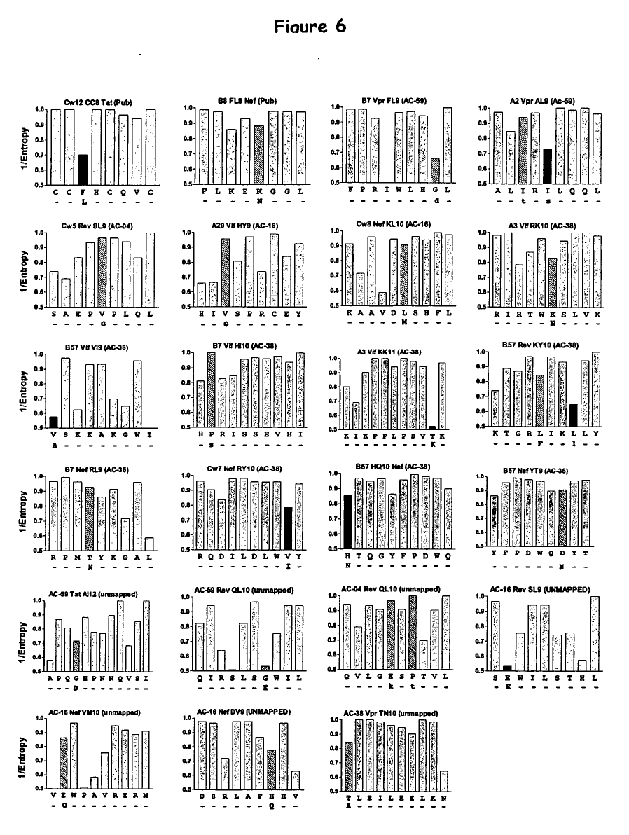

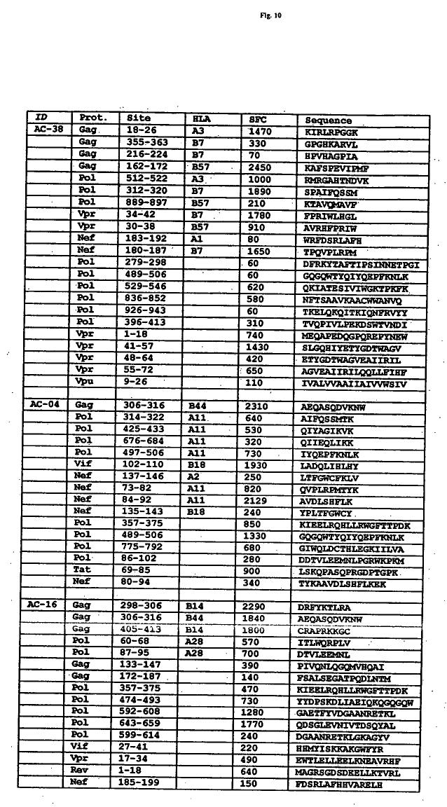

Peripheral blood mononuclear cells from subjects were also screened by IFN-γ Elispot assay to determine the specificity of the CD8+ T cell response to all non-envelope proteins. Where indicated, peptides corresponding to autologous virus sequences were also tested when the autologous virus differed from the clade B consensus sequence in a region undergoing evolution (See FIG. 9). Of the 98 mutations defined in these subjects, 29 resided within previously defined CD8 epitopes restricted by HLA alleles expressed in the individual subjects, and in each case a CD8+ T cell response was detected to the optimally defined CD8 epitope or the autologous variant (FIG. 2B). An additional 23 mutations were newly defined by IFN-γ Elispot assays to be targeted by circulating CD8+ T cells in these individuals, representing novel epitopes not previously described (FIGS. 2B, 5, and 9). Accordingly, 52 out of the 98 mutations (53% overall, range 48% to 60%) arising from the time of acute infection were linked to CD8 T cell selection pressures documented to be present in vivo.

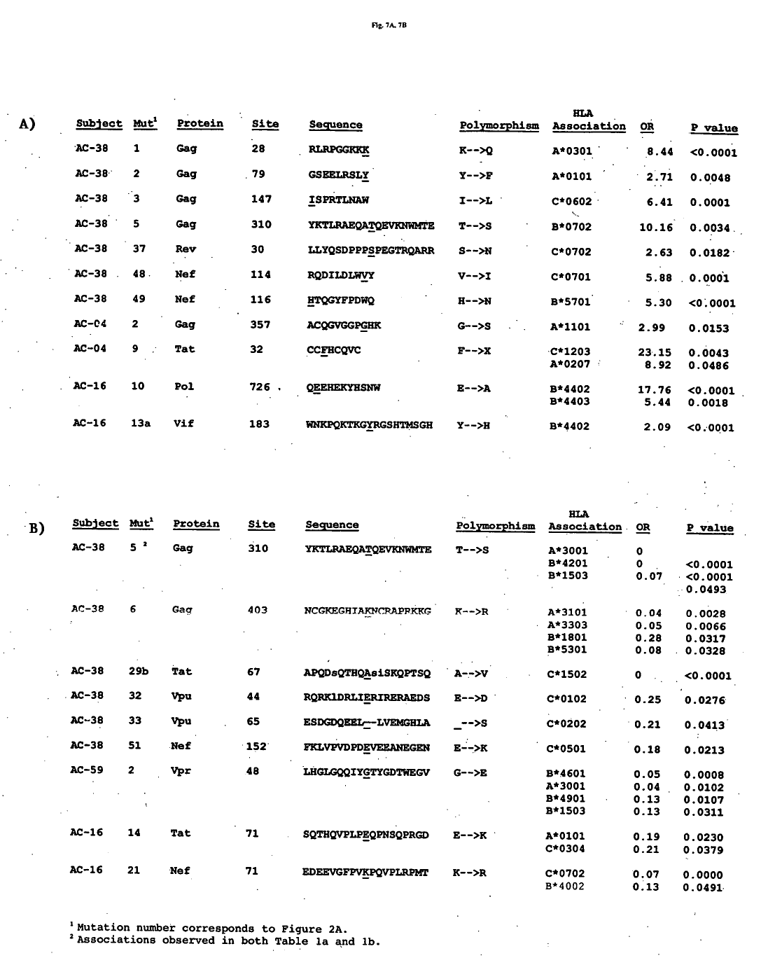

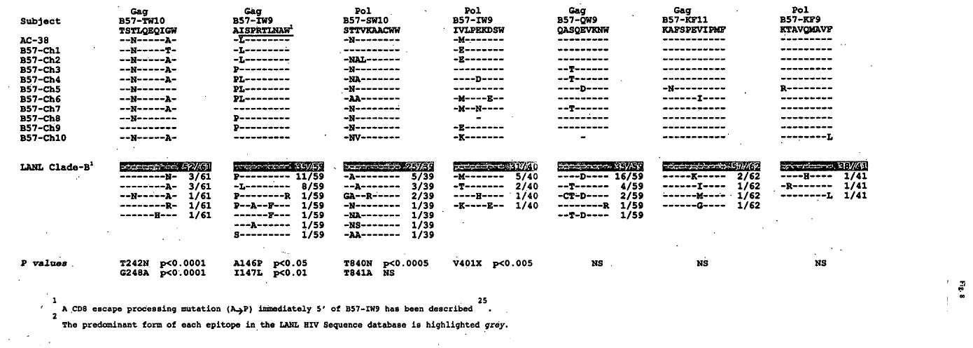

We next compared these evolving mutations to an extensive HIV-1 clade B sequence database in which allele-specific polymorphisms have been defined for the most frequently expressed alleles and statistically linked to immune selection pressure, but for which direct immunologic evidence has been lacking. Allele-specific polymorphisms were identified using the customized software program Epipop, as previously described (Moore et al., Science 296, 1439-43, 2002). Eleven mutations were strongly associated at the population level in persons expressing those same alleles (FIG. 7A), providing direct experimental support that these population sequence polymorphisms are linked to host CD8+ T cell responses.

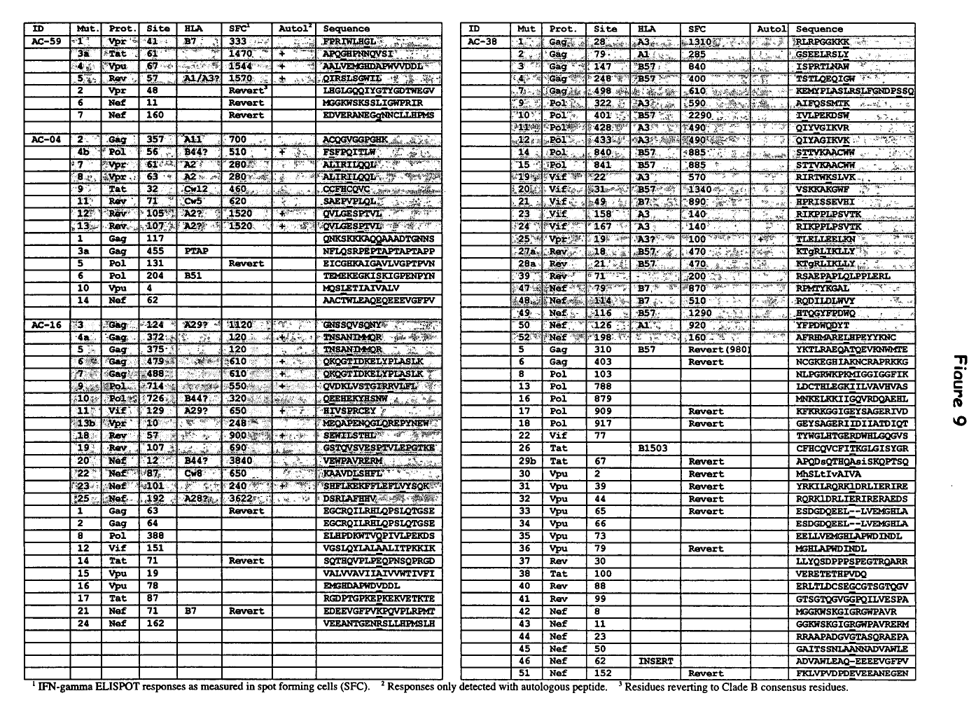

We also addressed the proportion of CD8+ T cell epitopes that remained invariant despite documented selection pressure in the three subjects followed for more than one year. In addition to the 21, 6, and 13 CD8+ T cell responses against evolving regions in subjects AC-38, AC-04 and AC-16, another 22, 16, and 16 responses were detected in each subject, respectively, that never varied despite persistence of responses (FIG. 10). Thus, a majority of the CD8+ T cell responses in these subjects (51%-73%) targeted regions that remained invariant over time. Including those epitopes that were associated with variation and those that remained stable, between 9% and 18% of the non-Env residues in the viral genomes of these subjects were exposed to CD8+ T cell selective pressures. Therefore, mutations were >7-fold more likely to occur within CD8 T cell-targeted versus non-targeted regions of the virus (p<0.0001), indicating that viral evolution was not random, but rather due to specific selection pressures. In addition to mutations arising under CD8 selection pressure, others arose in regions with no detectable CD8+ T cell responses. A possible cause of these sequence changes is reversion of transmitted mutations selected by CD8+ T cell responses in the prior host, which arise in the absence of the original selection pressure. Of the 46 mutations not associated with CD8+ T cell responses, 18 (39%) represented evolution towards the more common HIV-1 clade B consensus sequence, and most of these represented highly conserved residues in the database, consistent with mutations that are intrinsically unstable in the absence of selection pressure (FIG. 9). Again using Epipop, 9 out of the 18 mutations were found to be associated with allele-specific polymorphisms at the population level, and in each case the associated allele was not expressed by the respective acutely infected subject (FIG. 8B). Therefore, in addition to the 52 mutations associated with detectable CD8+ T cell responses in the host, at least 9 and as many as 18 additional mutations are consistent with adaptations due to previous CD8+ T cell selective pressures. Reversion of CD8-associated mutations following transmission may therefore be a common event and may additionally contribute to the evolution of HIV. Together with mutations associated with detectable CD8 responses, 62%-71% of all non-envelope mutations in these subjects were associated with cellular immune pressures (FIG. 2C). Based on the above data, the majority of mutations arising during HIV-1 infection may not simply be random, but rather may arise under specific selection pressures.

To further address the predictability of mutations, we examined whether precise sites in evolving epitopes are reproducibly selected and focused on HLA-B57, an allele known to be associated with enhanced immune control following acute infection, and therefore likely exerting substantial selective pressures 20. Each of the 12 previously defined HLA-B57-restricted CD8 epitopes 21 was targeted in untreated subject AC-38, and viral evolution was observed in 8 of these (FIG. 9). We focused on the seven B57 epitopes residing in Gag and Pol, as these conserved proteins were most likely to reveal clear evolutionary patterns. Viral evolution within these seven Gag and Pol epitopes was compared to viral evolution in ten additional HLA-B57 subjects exhibiting high levels of viral replication, which is clinically linked to disease progression 22. Four of these epitopes exhibited strikingly similar mutations at 7 residues, with 6 out of 7 residues significantly more variable (range p<0.05 to p<0.0001) in persons expressing HLA-B57 than in the LANL database overall (FIG. 8). A fifth epitope, QW9, exhibited only a low frequency of mutations at P3 and P5, whereas the remaining two epitopes, KF11 and KF9, were rarely observed to exhibit any sequence variation and were similarly highly conserved in the LANL database (FIG. 8). These data provide support for the concept of predictable viral evolutionary dynamics in persons sharing the same HLA allele.

### EXAMPLE 2

### Determination of Shannon Entropy Scores

Amino acid entropy scores were determined for each position of the HIV gag protein (TABLE 1) using Shannon's scoring system. A sampling of amino acid variations at the indicated position of the target protein is shown. The sampling may contain at least 5, 10, 20, 50, 100 or more different patient-derived isolates and may reflect data from clinical isolates taken from different times in a HIV infection or disease progression.

The nucleotide sequences were aligned and insertions were removed. The sequence was translated into the amino acid sequence and Shannon's entropy score was determined for each amino acid position in HIV proteins. TABLE 1 is organized as follows. Column A shows the amino acid number; column B shows the consensus (or most common) amino acid at a given position, column C shows the Shannon entropy score at each amino acid, therefore reflecting the variability of amino acids found in HIV at a position, and column E shows the raw data of amino acids found at a given position in the HIV data. Defined patient epitopes and escape mutations that evade the immune response are indicated. The collected data shows a correlation between escaped mutations and low entropy scores and suggest that escape mutations may be predicted from entropy scores.

The additional tables provide consensus sequences, entropy scores, and amino acid variants for Pol (TABLE 2), Vif (TABLE 3), Tat (TABLE 4), Rev (TABLE 5), Vpu (TABLE 6), Env (TABLE 7), Vpr (TABLE 8), and Nef (TABLE 9). As described above, target epitope domains are identified by an entropy score that is lower than the average entropy score. Based on the collected data, a vaccine is designed that includes a peptide having the consensus sequence as well as peptides having the consensus sequence with a sequence substitution at the position of the escape mutation.

### EXAMPLE 3

### Relationship Between Immune Pressure and HIV-1 Sequence Polymorphisms

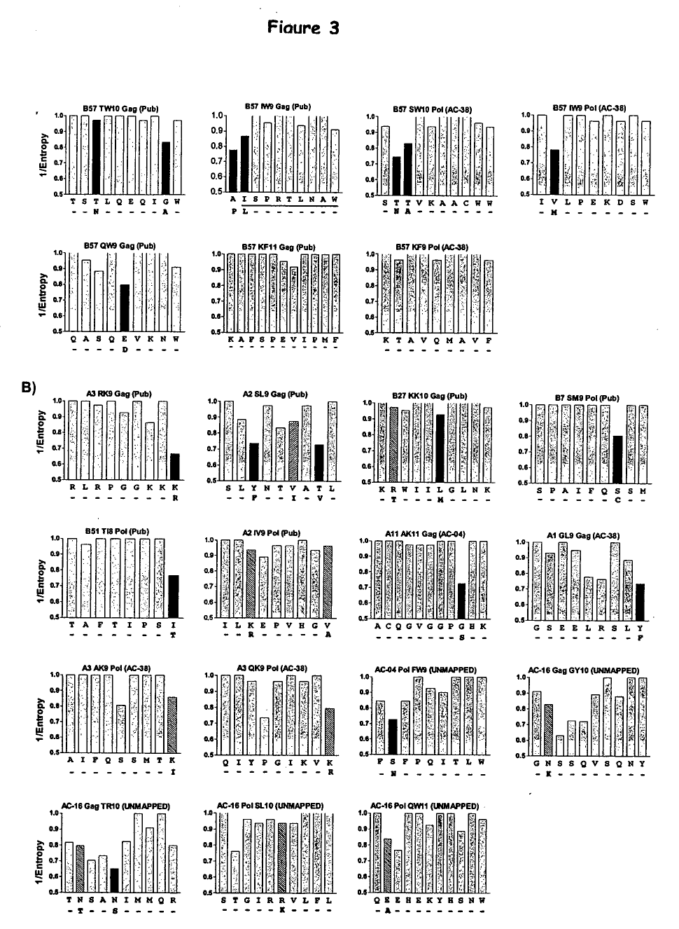

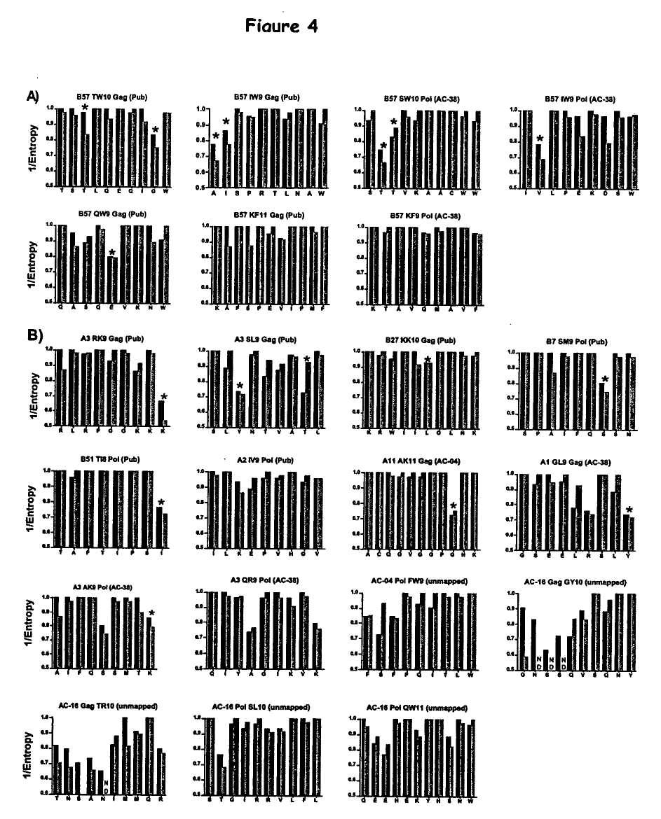

The reproducibility of mutations in the HLA-B57 epitopes suggested that there may exist a relationship between the mutations induced by immune pressures and HIV-1 sequence polymorphisms at the population level. We therefore determined the degree to which mutating and non-mutating residues within each CD8 epitope were associated with sequence polymorphisms at the population level. Using sequences reported in the Los Alamos National Laboratory (LANL) HIV Sequence Database (www.hiv.lanl.gov), Shannon entropy scores were determined for each residue of HIV-1 clade B, providing an overall assessment of the sequence diversity present at each residue in circulating HIV-1 clade B strains. FIG. 3A illustrates the relative conservation (1/entropy) of each residue within the seven HLA-B57-restricted CD8 epitopes studied and the position where mutations reproducibly arose. Not only did this analysis illustrate that the residues commonly mutating reflect the most polymorphic residues in each epitope, but also that the majority of non-mutating residues within these CD8 epitopes are highly conserved.

The analysis was then broadened to examine the additional CD8 epitopes defined in these four subjects, again focusing on responses to the more conserved structural proteins Gag and Pol. Overall, 18 of the 28 mutations (64%) in these epitopes were found to lie within the most variable residue in the epitope (FIG. 3; solid bars). One exception was the A3-AK9 Pol epitope (AIFQSSMTK (SEQ ID NO: 3); FIG. 3B) in which viral escape was observed at position 9 (P9) rather than at the most polymorphic P5 residue. Notably, a second epitope B7-SM9 (SPAIFQSSM (SEQ ID NO: 4); FIG. 3B) with an escape mutation at this same residue overlaps A3-AK9 and may account for this increased variability at P5 in the A3-AK9 epitope. Therefore, within these highly conserved proteins of HIV-1, the same residues are repeatedly selected for sequence variation, both in an individual and at the population level. Based on these results, selection against deleterious mutations (purifying selection) may function to conserve important residues in clade B while allowing sequence variation only at precise residues to facilitate evasion of host immune pressures.

As a result of these strong relationships, we conducted a similar analysis in the remaining more variable accessory and regulatory proteins of HIV-1. By combining previously described CD8 escape mutations in Tat and Nef, as well as evolving targeted CD8 epitopes in our study subjects, a total of 23 epitopes were examined. In this analysis, however, only 8/26 mutations correlated with the most variable residue in each epitope (FIG. 10). The inability to draw similar correlations between host CD8 escape mutations and sequence diversity in these proteins may be due to the greater sequence variability within these proteins. This increased variability may reflect either a more intense targeting of these regions of HIV-1 by host CD8+ T cell responses, or a reduced requirement to maintain such sequence conservation in these non-structural proteins.

In order to determine whether the inherent variability of a CD8 epitope was predictive of epitopes undergoing sequence variation, we compared the average entropy scores of mutating epitopes with those refractory to sequence changes using sequences from the LANL database. A comprehensive analysis of the 57 evolving and non-evolving minimally defined CD8 epitopes in this study, including those from the regulatory and accessory genes, demonstrated that epitopes exhibiting evolution were significantly less conserved than epitopes that remained unchanged (p<0.0005). Evolving epitopes exhibited average entropy scores of 90.5% per residue versus non-evolving epitopes exhibiting 94.8% per residue.

However, this finding may have been influenced by a trend for mutating epitopes to reside within the more variable non-structural proteins while conserved epitopes resided within the more conserved structural proteins (p=0.0531). Indeed, those proteins which accumulated the most mutations in these subjects when adjusting for protein length (Rev>Tat>Vpr>Vpu>Nef>others) closely reflect the relative variability of each of the HIV-1 proteins (Vpu>Tat>Rev>Nef>others) 24. Therefore, the inherent variability of a targeted CD8 epitope, or protein, appears to be influencing whether sequence variation is likely to occur in response to CD8+ T cell selective pressures.

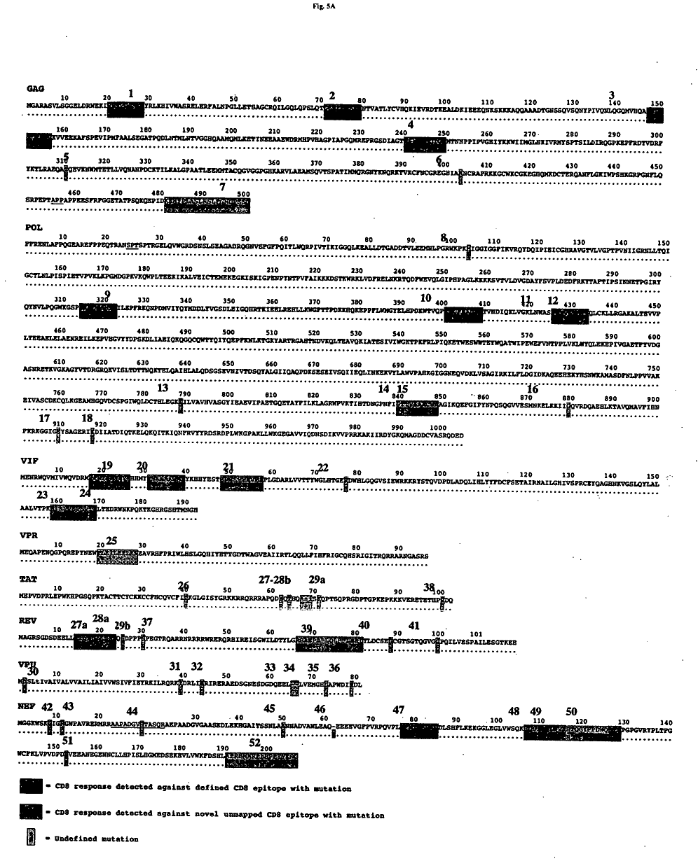

Restrictions may therefore influence the residues within a targeted region that are able to mutate. If purifying selective pressures play a role in dictating these restrictions, then similar patterns of conserved and variable residues may exist within other clades of HIV-1. Seventeen of the 19 mutations in the clade B CD8 epitopes in Gag and Pol which correlated with the most polymorphic residue at the population level (FIG. 3) also reflected the most polymorphic residue in clade C (FIG. 4). In fact, for the majority of residues in these epitopes very similar patterns of conserved and variable residues were observed between clade B and C, even at highly polymorphic residues (FIG. 4).

An analysis of CD8+ T cell-associated mutations in the accessory and regulatory proteins again did not exhibit any strong associations, although similar patterns of conserved and variable residues were still evident. Therefore, we broadened the analysis to all 2299 amino acid residues of HIV-1 (excluding Env). In total, 85% of residues exhibited similar entropy scores (within 10%) between clade B and C, despite over 45% of these 2299 residues exhibiting some degree of variability (>5%) in either clade B or clade C. Purifying selective pressures may therefore maintain the overall structure and function of HIV-1 across multiple clades while selectively allowing for the same residues to support sequence variation in the presence of host immune selective pressures.

Based on these data assessing the impact of CD8+ T cell selective pressures on HIV-1 evolution, nearly two thirds of all mutations arising in non-envelope proteins in the transition to chronic infection may be attributed to either CD8-associated selective pressures in the infected individual, or reversion of HLA class I-associated mutations selected in a previous host. Viral evolution following acute HIV-1 infection is thus not a random process, but rather is substantially influenced by adaptive CD8+ T selection pressures. Given the preferential selection of individual residues for mutation and the repeated selection for identical mutations, there may significant constraints on viral evolution and explain the ability to repeatedly link certain sequence polymorphisms in HIV-1 with various HLA alleles, despite both HIV-1 and the HLA class I loci being highly polymorphic. Moreover, due to the selective mutation of specific residues within epitopes and concordance of highly variable sites among different lades, immune-selection may also drive global HIV diversity. A substantial proportion of the IV proteome may therefore be under immune selection pressure, and a majority of CD8 responses may not result in sequence changes within the targeted epitopes despite nearly 20% of all residues being targeted in some persons. Since the non-mutating residues are more highly conserved at a population level, these data indicate substantial constraints on HIV evolution. Indeed, comparing the entropy values of clade B and clade C sequences indicated very similar patterns of conserved and variable residues, suggesting that viral escape in different HIV-1 clades may occur at similar residues despite unique distributions of HLA alleles present in infected populations.

Accordingly, the same purifying selection pressures may contribute to maintain specific residues and preserve the basic structure of HIV-1 proteins in different HIV-1 clades, while routinely allowing other residues to fluctuate. Indeed, the same residues observed to mutate in CD8 epitopes in clade B and C reflect those consistently capable of supporting alternative amino acids in many other HIV-1 clades including clade O and CPZ, whereas conserved residues are maintained across different HIV-1 clades.

The methods used to detect CD8+ T cell responses relied on measuring IFN-γ production. If desired, in vitro expansion or alternative measurements of CD8+ T cell immune function such as cytolytic capacity, TNF-α production, or HLA class I tetramers Shankar, P. et al., Blood 96: 3094-101, 2000; Kostense, S. et al. Blood 99: 2505-11, 2002) may also be employed. It is noteworthy that four of the mutations not associated with CD8+ T cell responses are within regions exhibiting CD4+ T cell proliferative responses. Compensatory mutations accommodating CD8 escape mutations and antigen processing mutations may also account for additional sequence polymorphisms.

### EXAMPLE 4

### Design of Vaccine

Formulation 1

A vaccine composition based on the data obtained above includes a linear gag peptide corresponding to at least 8 consecutive residues of the consensus sequence in TABLE 1. Position 12 is occupied by any of the residues observed at that position in the patient database, such as E, K, Q, G, or D. The composition contains one or a mixture of peptide, e.g., 6 peptides, each with a different residue at position 12.

Formulation 2

A vaccine composition may include a linear gag peptide corresponding to at least 8 consecutive residues of the consensus sequence in TABLE 1. Position 28 is occupied by any of the residues observed at that position in the patient database, such as K, H, R, Q, T, or M. The composition contains one or a mixture of peptides.

Methods and Materials

Subjects

The four acute HIV-1 infected subjects were enrolled from the Boston Acute Infection Cohort and identified during primary HIV-1 infection. Subjects AC-04 and AC-16 were enrolled in a structured treatment interruption protocol previously described (Rosenberg et al., Nature 407: 523-6, 2000). Chronic infected subjects were enrolled from the Massachusetts General and Shattuck Hospitals in Boston, Queen Elisabeth Hospital in Bridgetown, Barbados 41, and Praxis Heiko Jessen, Berlin. HLA class I typing of four study subjects were as follows: AC-59 (A02, B07, B44, C05, C07); AC-04 (A02, A11, B18, B44, C05, C12); AC-38 (A01, A03, B07, B57, C06, C07); AC-16 (A28, A29, B14, B44, C08). Time points for each subject include: AC-38 (day 64, 408, 1073, 1213, 1510), AC-59 (day 41, 111), AC-04 (day 0, 1650), AC-16 (day 0, 1934) defined from day of presentation with acute HIV-1 infection.

IFN-γ Elispot Assay

HIV-1-specific CD8+ T-cell responses were quantified by IFN-γ Elispot assay as previously described 16, using overlapping peptides (15-18mer peptides overlapping by 10 amino acids) spanning the entire expressed HIV-1 clade B 2001 consensus sequence, as well as peptides corresponding to optimal described clade B CTL epitopes 21 and autologous virus sequences. IFN-γ secreting T-cells were counted by automated reader and expressed as spot forming cells (SFC) per 106 input cells after subtracting the negative control. A response was considered positive if >50 SFC per 106 cells and at least 3 times greater than mean background activity. Comparison of autologous and variant epitopes was performed using log dilutions of peptides as described (Altfeld et al., Nature 420: 434-9, 2002).

Sequencing of Autologous Virus

Viral DNA was isolated from PBMC (5×106 cells) and viral RNA was extracted from plasma samples and reverse-transcribed as described previously 17. A set of 49 primary and nested PCR primer pairs amplified the entire HIV-1 genome as previously described 17. When necessary PCR fragments were gel purified, and PCR products were then population sequenced bi-directionally on an ABI 3100 PRISM automated sequencer. Sequencher (Gene Codes Corp., Ann Arbor, Mich.) and MacVector 4.1 (Oxford Molecular) software were used to edit and align sequences. These sequence data are available from GenBank under accession no. xxxx to xxxx.

Identification of HLA-Associated Sequence Polymorphisms

HLA-associated sequence polymorphisms were identified using the customized software program Epipop, as previously described (Moore et al., Science 296: 1439-43, 2002).

Statistical Analysis

Statistical analysis and was done using Prism 4.0 (GraphPad, San Diego, Calif.).

All publications and patents cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Other embodiments are within the following claims.

Sample#: 62

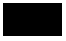

Gag

| | | | |
|---|---|---|---|
| AA # | Cons. | Conservation | Raw data |

Table 1A

| | | | |
|---|---|---|---|
| 1 | M | 0.973 | #MMMMMMMMMMMMMMMMMMMMMMMMMMMMMMMMMMMMMLMMMMMMMMMMMMMMMMMMMMMMMMM |
| 2 | G | 1 | #GGGGGGGGGGGGGGGGGGGGGGGGGGGGGGGGGGGGGGGGGGGGGGGGGGGGGGGGGGGGGGG |
| 3 | A | 1 | #AAAAAAAAAAAAAAAAAAAAAAAAAAAAAAAAAAAAAAAAAAAAAAAAAAAAAAAAAAAAAAA |
| 4 | R | 1 | #RRRRRRRRRRRRRRRRRRRRRRRRRRRRRRRRRRRRRRRRRRRRRRRRRRRRRRRRRRRRRRR |
| 5 | A | 1 | #AAAAAAAAAAAAAAAAAAAAAAAAAAAAAAAAAAAAAAAAAAAAAAAAAAAAAAAAAAAAAAA |
| 6 | S | 1 | #SSSSSSSSSSSSSSSSSSSSSSSSSSSSSSSSSSSSSSSSSSSSSSSSSSSSSSSSSSSSSSS |
| 7 | V | 0.856 | #VVVIVVVVVVVVVVIIIVVVVVVVVVIVVVVVVVVVVVVIVVVIVIVVVVVVVVVVVIVVVVI |
| 8 | L | 0.954 | #LLLLLLLLLLLLLLLLLLLLLLLLLLLLLLLILLLLILLLLLLLLLLLLLLLLLLLLLLLLLL |
| 9 | S | 0.973 | #SSSSSSSSSSSSSSSSSSSSSSSSSSSSSSSSSSSSSSSSSSSSSSSSSSSSSSSSSSSSSSK |
| 10 | G | 0.954 | #GGGGGGGGGGGGGGGGGGGGGGGGGGGGGGGGGGGGGGGGGGGGGAGGGGGGGGGAGGGGGGG |
| 11 | G | 0.954 | #GGGGGGGGGGGGGGGGGEGGGGGGGGGGGGGGGGGGGGGGGGGGGGGGGGGGGGGGGGGGGGE |
| 12 | E | 0.691 | #EKKEEEEEEEKEEEKEEEKQQQQQEEGEEEEEEEEEEEEQEEEEEEDEKEEEEKKEKEEEKQK |
| 13 | L | 1 | #LLLLLLLLLLLLLLLLLLLLLLLLLLLLLLLLLLLLLLLLLLLLLLLLLLLLLLLLLLLLLLL |
| 14 | D | 1 | #DDDDDDDDDDDDDDDDDDDDDDDDDDDDDDDDDDDDDDDDDDDDDDDDDDDDDDDDDDDDDDD |
| 15 | R | 0.742 | #RKKRRRKRRRRRRRRRRRKRRRRRRKRRKKRRKRKRRRKRKRRQKKRKKKRKRRKKKKRRSRR |
| 16 | W | 0.973 | #WWWWWWWWWWWWWWWWWWWWWWWWWWWWWWWW$WWWWWWWWWWWWWWWWWWWWWWWWWWWWWW |
| 17 | E | 1 | #EEEEEEEEEEEEEEEEEEEEEEEEEEEEEEEEEEEEEEEEEEEEEEEEEEEEEEEEEEEEEEE |
| 18 | K | 0.884 | #KKKKKKKKKKKKKRKKKKKKKKKKKKKKKKKKKKTKKKKKKKKKKRKKKKNKKKKKKKKKKKR |
| 19 | I | 0.973 | #IIIIIIIIIIIIIIIIIIIIIIIIIIIIIIIIIIIIIIIIIIIIIVIIIIIIIIIIIIIIIII |
| 20 | R | 1 | #RRRRRRRRRRRRRRRRRRRRRRRRRRRRRRRRRRRRRRRRRRRRRRRRRRRRRRRRRRRRRRR |
| 21 | L | 1 | #LLLLLLLLLLLLLLLLLLLLLLLLLLLLLLLLLLLLLLLLLLLLLLLLLLLLLLLLLLLLLLL |
| 22 | R | 0.973 | #RRRRRRTRRRRRRRRRRRRRRRRRRRRRRRRRRRRRRRRRRRRRRRRRRRRRRRRRRRRRRRR |
| 23 | P | 1 | #PPPPPPPPPPPPPPPPPPPPPPPPPPPPPPPPPPPPPPPPPPPPPPPPPPPPPPPPPPPPPPP |
| 24 | G | 0.927 | #GGGGGGGGGGGGGRGGGGGGGGGGGGGGGGGGEGGGGGGGGGGGGGGGRGGGGGGGGGGGGGG |
| 25 | G | 1 | #GGGGGGGGGGGGGGGGGGGGGGGGGGGGGGGGGGGGGGGGGGGGGGGGGGGGGGGGGGGGGGG |
| 26 | K | 0.865 | #KKKKKKKKKRKKKKKKKRKKKKKKKKKKKKKSKKKRSKKKKKKKKKKKKKKKKKKKKKKNKKK |
| 27 | K | 1 | #KKKKKKKKKKKKKKKKKKKKKKKKKKKKKKKKKKKKKKKKKKKKKKKKKKKKKKKKKKKKKKK |
| 28 | K | 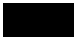 | #KHHKRQKKKKKTQKKKKKTKKKKKKKKKKKKKKKQKKKQRKKKKRKKLRKKQKKKQKKKKKMH |
| 29 | Y | 1 | #YYYYYYYYYYYYYYYYYYYYYYYYYYYYYYYYYYYYYYYYYYYYYYYYYYYYYYYYYYYYYYY |
| 30 | K | 0.589 | #KKKRKRKKKKKKRQKQQQQRLQRRRQRKQKRRHRRRRRKRQRKRKRMKKKKRKKRRKRKKKKM |
| 31 | L | 1 | #LLLLLLLLLLLLLLLLLLLLLLLLLLLLLLLLLLLLLLLLLLLLLLLLLLLLLLLLLLLLLLL |
| 32 | K | 1 | #KKKKKKKKKKKKKKKKKKKKKKKKKKKKKKKKKKKKKKKKKKKKKKKKKKKKKKKKKKKKKKK |
| 33 | H | 1 | #HHHHHHHHHHHHHHHHHHHHHHHHHHHHHHHHHHHHHHHHHHHHHHHHHHHHHHHHHHHHHHH |
| 34 | I | 0.759 | #IIIVIIIVVIIIIIIIIIILVILLIILIIIVIILIIIIILIIILILIIIIVIIIIILLIIIII |
| 35 | V | 0.973 | #VVVVVVVVVVVVVVVVVVVVVVVVVVVVVVVVVVVVVVVVVVVVVVVIVVVVVVVVVVVVVVV |
| 36 | W | 0.973 | #WWWWWWWWWWWWWWWWWWWWWWWWWWWWWWWW$WWWWWWWWWWWWWWWWWWWWWWWWWWWWWW |
| 37 | A | 1 | #AAAAAAAAAAAAAAAAAAAAAAAAAAAAAAAAAAAAAAAAAAAAAAAAAAAAAAAAAAAAAAA |
| 38 | S | 0.947 | #SSSSRSSSSSSSSSSSSSSSSGSSSSSSSSSSSSSSSSSSSSSSSSSSSSSSSSSSSSSSSSS |
| 39 | R | 1 | #RRRRRRRRRRRRRRRRRRRRRRRRRRRRRRRRRRRRRRRRRRRRRRRRRRRRRRRRRRRRRRR |
| 40 | E | 1 | #EEEEEEEEEEEEEEEEEEEEEEEEEEEEEEEEEEEEEEEEEEEEEEEEEEEEEEEEEEEEEEE |
| 41 | L | 1 | #LLLLLLLLLLLLLLLLLLLLLLLLLLLLLLLLLLLLLLLLLLLLLLLLLLLLLLLLLLLLLLL |
| 42 | E | 1 | #EEEEEEEEEEEEEEEEEEEEEEEEEEEEEEEEEEEEEEEEEEEEEEEEEEEEEEEEEEEEEEE |
| 43 | R | 0.947 | #RRRRRRRRRRRRRRRRRRRRRRRRRRRRRRRRRRRRRRRRRRRRRRRRRRRRRRRRRQRRRRK |
| 44 | F | 0.954 | #FFFFFFFFFFFFFFFFFFFFLFFFFFFFFFFFLFFFFFFFFFFFFFFFFFFFFFFFFFFFFFF |
| 45 | A | 0.947 | #AAAAAAAAAATAASAAAAAAAAAAAAAAAAAAAAAAAAAAAAAAAAAAAAAAAAAAAAAAAAA |
| 46 | V | 0.861 | #VLLVVVVVVVVVVVVVVVVVVVVVVVVVIVVVVVVVVIVVIVVVVVVVVVVVVVVVVIVVVVL |

Table 1B

| | | | |
|---|---|---|---|
| 47 | N | 0.973 | #NNNNNNNNNNNNNNNNNNNNNNNNNNNNNNNNNNNNNNNNNNNNNNNNNNNNNNNDNNNNNNN |
| 48 | P | 0.973 | #PPPPPPPPPPLPPPPPPPPPPPPPPPPPPPPPPPPPPPPPPPPPPPPPPPPPPPPPPPPPPPP |
| 49 | G | 0.937 | #GGGGGGGGGGGGGGGGGGGGGGGGGGGGGGGGGGGGGGGGGGSGGGGGGGGGGGGGGGSGGGG |
| 50 | L | 1 | #LLLLLLLLLLLLLLLLLLLLLLLLLLLLLLLLLLLLLLLLLLLLLLLLLLLLLLLLLLLLLLL |
| 51 | L | 1 | #LLLLLLLLLLLLLLLLLLLLLLLLLLLLLLLLLLLLLLLLLLLLLLLLLLLLLLLLLLLLLLL |
| 52 | E | 1 | #EEEEEEEEEEEEEEEEEEEEEEEEEEEEEEEEEEEEEEEEEEEEEEEEEEEEEEEEEEEEEEE |
| 53 | T | 0.954 | #TTTTTTTTTTTTTTTTTTTTTSTTTTTTTTTTTTTTTTTTTTTTTTTTTTTTTTSTTTTTTTT |
| 54 | S | 0.813 | #SSSASSSAASSSSSSSSSGSSSSSSSSSSSSSSSSSSTSSSSSSASASASSSSSSSAASSSSA |
| 55 | E | 0.813 | #EEEALDQEEEEEEEEEEEGEEEEEEEEEEEEEEEQDEEEDEEEEEEEEEEEEEEEEEEEEEEE |
| 56 | G | 1 | #GGGGGGGGGGGGGGGGGGGGGGGGGGGGGGGGGGGGGGGGGGGGGGGGGGGGGGGGGGGGGGG |
| 57 | C | 1 | #CCCCCCCCCCCCCCCCCCCCCCCCCCCCCCCCCCCCCCCCCCCCCCCCCCCCCCCCCCCCCCC |
| 58 | R | 0.779 | #RIRRQRIKKRRRRRRRRRKRRRRKRRRRRRRKRKRRKRRKRRRRRRRRRRRRRRRRRRRRRRN |
| 59 | Q | 0.973 | #QQQQKQQQQQQQQQQQQQQQQQQQQQQQQQQQQQQQQQQQQQQQQQQQQQQQQQQQQQQQQQQ |
| 60 | I | 1 | #IIIIIIIIIIIIIIIIIIIIIIIIIIIIIIIIIIIIIIIIIIIIIIIIIIIIIIIIIIIIIII |
| 61 | L | 0.877 | #LLLLLLMIILLLLLLLLLLLLLLLLMLLLLLMLLLLLLLLLLLLLLLLLLLLLLLLLLLLLLM |
| 62 | G | 0.614 | #GKKGGRRGGGVVGRGGEGVEQEEEGGGGGGEGGEEGGGGAGGGGGGEGGGGRGGGGGGGGGGN |
| 63 | Q | 0.973 | #QQQQQQQQQQQQQQQQQQQQQQQQQQQQQQQQRQQQQQQQQQQQQQQQQQQQQQQQQQQQQQQ |
| 64 | L | 1 | #LLLLLLLLLLLLLLLLLLLLLLLLLLLLLLLLLLLLLLLLLLLLLLLLLLLLLLLLLLLLLLL |
| 65 | Q | 0.911 | #QQQQQQQQQQQQQQHQQHQQQQQQQQQQQQQQQQQQQQQHQQQQQQQQQQQQQQLQQQQQQQQ |
| 66 | P | 0.897 | #PPPPPPPSSPPPPPPPPPPPPPPPPPPPPPPPPPPPPPPPSPPPPSPPPPPPPPPPPSSPPPP |
| 67 | S | 0.741 | #SSSSSSSSSSSSSASAATSASSSSSSASSSSSSASTASAAAASSASSSASSSASASSASSSVA |
| 68 | L | 0.974 | #LLLLLLLLLLLLLLLLLLLLLLLLLLLLLLLLLLLLLILLLFLLLLLLLLLLLLLLLLLLLLL |
| 69 | Q | 0.837 | #QQQQYQQQQQQPKQQQQPQQQQQQQQQQKQQQKQQQQQQKQQQQQQQQQQQQQQKQQQQQQQQ |
| 70 | T | 1 | #TTTTTTTTTTTTTTTTTTTTTTTTTTTTTTTTTTTTTTTTTTTTTTTTTTTTTTTTTTTTTTT |
| 71 | G | 1 | #GGGGGGGGGGGGGGGGGGGGGGGGGGGGGGGGGGGGGGGGGGGGGGGGGGGGGGGGGGGGGGG |
| 72 | S | 0.931 | #STTSSSSSSSSSSSSSSSSSSSSSSSSSSSSSSSSSSSSSSSSSSSSSSSSSSSSSSSSSSST |
| 73 | E | 1 | #EEEEEEEEEEEEEEEEEEEEEEEEEEEEEEEEEEEEEEEEEEEEEEEEEEEEEEEEEEEEEEE |
| 74 | E | 0.949 | #EEEEEEEEEEEEEDEEDEEEEEEEEEEEEEEEEEEEEEEEEEEEEEEEEEEEEEEEEEEEEEE |
| 75 | L | 0.777 | #LIILLLLLLLLLIFLLLLLLLLLLLLLLILRLLLLLLLLLILLLLLLLLLLRLLLLLLLLLLL |
| 76 | R | 0.763 | #RKKKKRKKKKKKKRKRRKKRRRRRRRRRRRRRKRRRRRRRRRKKRRRRKRKRRKRRKRKRRRK |
| 77 | S | 1 | #SSSSSSSSSSSSSSSSSSSSSSSSSSSSSSSSSSSSSSSSSSSSSSSSSSSSSSSSSSSSSSS |
| 78 | L | 0.886 | #LLLLLLLLLLLLLLVLVLLLLLLLLLLLLLLLLLLLLLLVLLLLLLLLLLLLLLLLLLLLLLL |
| 79 | Y | 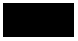 | #YHHYFYYFFFYFYYYYYYYFFFFFFYYYYYFFYYYYFYYYYYFFFYYYYYYFHFYYYYYYYYF |
| 80 | N | 0.973 | #NNNNNNYNNNNNNNNNNNNNNNNNNNNNNNNNNNNNNNNNNNNNNNNNNNNNNNNNNNNNNNN |
| 81 | T | 0.834 | #TTTTTTTTTTTTTTATTTATTTLLTTTTTTTVTTLTTTTTTTTATTTTATTTTTTTTTTTTTT |
| 82 | V | 0.875 | #VVVVIVVVVVVVVVVVVVVVVVIIVVVVVVVVVIVVVVVVVVVVVVIIVVVVVVVVIVVVVIV |
| 83 | A | 0.973 | #AAAAVAAAAAAAAAAAAAAAAAAAAAAAAAAAAAAAAAAAAAAAAAAAAAAAAAAAAAAAAAA |
| 84 | T | 0.729 | #TTTTVTTVVVVTTTVTTVTTTVAATTTTTTTVTTTAVVTTVTTVVTTATTVVTTTTVTTVTVT |
| 85 | L | 1 | #LLLLLLLLLLLLLLLLLLLLLLLLLLLLLLLLLLLLLLLLLLLLLLLLLLLLLLLLLLLLLLL |
| 86 | Y | 1 | #YYYYYYYYYYYYYYYYYYYYYYYYYYYYYYYYYYYYYYYYYYYYYYYYYYYYYYYYYYYYYYY |
| 87 | C | 1 | #CCCCCCCCCCCCCCCCCCCCCCCCCCCCCCCCCCCCCCCCCCCCCCCCCCCCCCCCCCCCCCC |
| 88 | V | 0.973 | #VVVVVVVVVVVVVVVVVVVVVVVVVVVVVVVVVVVVVVVVVVVVVIVVVVVVVVVVVVVVVVV |
| 89 | H | 1 | #HHHHHHHHHHHHHHHHHHHHHHHHHHHHHHHHHHHHHHHHHHHHHHHHHHHHHHHHHHHHHHH |
| 90 | Q | 0.832 | #QQQQQQQQQQQQQQQQQQQLQNNNQQQQQQQQQQQQQQQQQQQQQQQQQQQQQQQQQQQQERK |
| 91 | R | 0.672 | #RRRKRRRRRKGKKRNRRRGRRRRRRKKRKKRRKRKKKKKRRRRRRKKRNRKRRNRKMRRKRRK |
| 92 | I | 0.947 | #IIIIIIIIIIIIIIIIILIIMIIIIIIIIIIIIIIIIIIIIIIIIIIIIIIIIIIIIIIIIII |
| 93 | E | 0.799 | #EDDDDDEDDDEEEEDEEEEEEEEEEEDEEDDEEEEEEEEEDEEEEEEDEDEDEDEEDEEEEED |
| 94 | V | 0.808 | #VVVVVVVVVVVIVVIIVVVIIIIIVVVIVVVIIVVIVVVIIVVIVIVVVVIVVVVVVVVVVVV |
| 95 | K | 0.822 | #KRRRKREKKRKRKKKKKKRKKKKKKKKKKKKKKKRKKKKKKKKKKKKKRKKKKRKKKKKKKKR |
| 96 | D | 1 | #DDDDDDDDDDDDDDDDDDDDDDDDDDDDDDDDDDDDDDDDDDDDDDDDDDDDDDDDDDDDDDD |
| 97 | T | 1 | #TTTTTTTTTTTTTTTTTTTTTTTTTTTTTTTTTTTTTTTTTTTTTTTTTTTTTTTTTTTTTTT |

Table 1C

| | | | |
|---|---|---|---|
| 98 | K | 0.927 | #KKKKKKKKKKKKKKKKKKKKKKPQKKKKKKKQKKKKKKKKKKKKKKKKKKKKKKKKKKKKKKK |
| 99 | E | 0.973 | #EEEEEEEEEEEEEEEEEEEEEEEEEEEEEEEEEEEEEEEEEEEEEDEEEEEEEEEEEEEEEEE |
| 100 | A | 1 | #AAAAAAAAAAAAAAAAAAAAAAAAAAAAAAAAAAAAAAAAAAAAAAAAAAAAAAAAAAAAAAA |
| 101 | L | 0.973 | #LLLLLLLLLLLLLLLLVLLLLLLLLLLLLLLLLLLLLLLLLLLLLLLLLLLLLLLLLLLLLLL |
| 102 | E | 0.75 | #EDDEEDDEEEDEEEEEEEDEEEEEDDDDDEDNDEDDDEEDDDEEDEEDDEEDEDEEEEDDDDD |
| 103 | K | 0.922 | #KKKRKKKKKRKKKRKKKKKKKKKKKKKKKKKKKKKKKRKKKKKKKKKKKKKKKKKKKKKKKKK |
| 104 | I | 0.922 | #IIIIIIIIIVIIIVIIIIIIIIIIIVIIIIIIIIIIIIIIIIIIIIIIIIIIIIIIIIIIVII |
| 105 | E | 0.973 | #EEEEEEEEEEEEEEEEEEEEEEEEEEEEEEEEEEEEEEEEEEEEEEEEEEEEEEEEEEEEEQE |
| 106 | E | 1 | #EEEEEEEEEEEEEEEEEEEEEEEEEEEEEEEEEEEEEEEEEEEEEEEEEEEEEEEEEEEEEEE |
| 107 | E | 1 | #EEEEEEEEEEEEEEEEEEEEEEEEEEEEEEEEEEEEEEEEEEEEEEEEEEEEEEEEEEEEEEE |
| 108 | Q | 0.901 | #QQQQQQQQQQQQQQQQQQQQHQHPQQQQQQQQQRQQQQQQQQQQQQQQQQQQQQQQQQQQQQQ |
| 109 | N | 0.884 | #NNNNNNNNNNNNNNNNNNNNNNNNNNNNNNSNNNNNSNNNDNNSNNNNNNNNNNANNNNNNNN |
| 110 | K | 0.954 | #KKKKKKKKKKKKKKKKKKKKKKKKKNKKKKKKKKKKKKKKKKKKKKKKKKKKKKKKNKKKKKK |
| 111 | S | 0.865 | #SSSSSCSYYSSSSSCSSCSSSSSSSSSSSSSSSSSSSISSSSSSSSSSSSSSSSSSSSSSSSS |
| 112 | K | 0.92 | #KKKKKKNKKKKKKKKKKKKKKKKKKKKK$KKKKKKKKKKKKKKKKKKKKKKKKKKKKKKKKKQ |
| 113 | K | 0.877 | #KQQKKKKKKKKKKEKKKKKKEKKKKKKKKKKKQKKKKKKKKKKKKKKKKKKKKKKKKKKKKKQ |
| 114 | K | 0.891 | #KKKKKKKMMKKKKKKKKKKKKKKKKKKKKKKKKKKRRRKKKKKKKKKKKKKKKKKKKKKKKKK |
| 115 | A | 0.862 | #AAAAAAAAAAAAAAATAAAAAAAAAAAAAAAAAAVAAAAAAAAAAAVAAAAAAAEAAAAAAET |
| 116 | Q | 0.947 | #QQQQQQHQQQQQQQQQQQQQQQQQQQQQQQQQQQQQQQQQQQQQQQQQQQQQQQ-QQQQQQQQ |
| 117 | Q | 0.881 | #QQQQQQQTTQPQQQQQQQQQQQQQPQQQQQQQQQQQQQQQQQQQQQQQQQQQQQ-QQQQQQQQ |
| 118 | A | 0.911 | #AAAAAAAAAAAAAAAAAAATATAAAEAATAAAAAAAAAAAAAAAAAAAAAAAAAAAAAAAAAA |
| 119 | A | 0.927 | #AAAAAAAAAAAAAAAAAAAAAAAAAAAAAAAAAAAAATAAAAATAAAAAAAAAAAAAAAAAAK |
| 120 | A | 0.947 | #AAAAAAAAAAAAAAAAAAAAAAAARAAAAAAAAAAAAAAAAAAAATAAAAAAAAAAAAAAAAA |
| 121 | D | 0.745 | #DDDDDDDGGSDDDDGGGGDGDDCDDDDDDGDDDDDDDDDDDDDDDGADDADDDDDDDGDDDDA |
| 122 | T | 0.774 | #TTTTTTTTTTTATTTTPTTTKITTAATTTTKTATTTTTTTTTTTTTTTTATTTATTTTTTTTD |
| 123 | G | 0.909 | #GGGGGGGGGGGGEGGGGGGGGGGGGGGGGGGEGGGGGGGGGGGGGGGGGGGGGEGGGGGEGGE |
| 124 | N | 0.829 | #NNNNNNHNNNNNNNNNNNNNNNNNNNNHNNNNNNNNNNNNNNNSNNTNNTNNNKNNNNNNNN- |
| 125 | S | 0.629 | #SSSSDSNSSTSSSSSSSSSNNNNNNRSSSSSNSSSSNSSSSKSSSNGNGGRSSSSSSSSRSS- |
| 126 | S | 0.735 | #SNNSGSKSSASSSSNSSSSSSSSSSNSNSSNSSSSSSSSSNSSSSSNSSNGSSSSSSSSNSS- |
| 127 | Q | 0.72 | #QQQQPQQQQQQKQQPQQQQQQPPQQQQQQQQQKQVQQKQQPMKQQPQQQQQQKQQQQNQQQQK |
| 128 | V | 0.891 | #VAVAVVVIIVVVVVVVVVVAVVVVVVVVVVVVVVVVVVVVVVVVVVVVVVVVVVVVVVVVVVV |
| 129 | S | 1 | #SSSSSSSSSSSSSSSSSSSSSSSSSSSSSSSSSSSSSSSSSSSSSSSSSSSSSSSSSSSSSSS |
| 130 | Q | 0.881 | #QQQQQQQQQHQQQQQQQQQQQQPQQQQQQQQQQQQQQHRQQQQQQQQQQQQQQQQQRQQQQQQ |
| 131 | N | 1 | #NNNNNNNNNNNNNNNNNNNNNNNNNNNNNNNNNNNNNNNNNNNNNNNNNNNNNNNNNNNNNNN |
| 132 | Y | 1 | #YYYYYYYYYYYYYYYYYYYYYYYYYYYYYYYYYYYYYYYYYYYYYYYYYYYYYYYYYYYYYYY |
| 133 | P | 1 | #PPPPPPPPPPPPPPPPPPPPPPPPPPPPPPPPPPPPPPPPPPPPPPPPPPPPPPPPPPPPPPP |
| 134 | I | 1 | #IIIIIIIIIIIIIIIIIIIIIIIIIIIIIIIIIIIIIIIIIIIIIIIIIIIIIIIIIIIIIII |
| 135 | V | 1 | #VVVVVVVVVVVVVVVVVVVVVVVVVVVVVVVVVVVVVVVVVVVVVVVVVVVVVVVVVVVVVVV |
| 136 | Q | 1 | #QQQQQQQQQQQQQQQQQQQQQQQQQQQQQQQQQQQQQQQQQQQQQQQQQQQQQQQQQQQQQQQ |
| 137 | N | 1 | #NNNNNNNNNNNNNNNNNNNNNNNNNNNNNNNNNNNNNNNNNNNNNNNNNNNNNNNNNNNNNNN |
| 138 | L | 0.742 | #LILLILLLLLLLLMMIIILLLLLLLLIILLLLMLLLLLILLLILLLLLLLILILMLILILIML |
| 139 | Q | 0.947 | #QQQQMQQQQQQQQQQQQQQQQQQQQQQQQQQQQQQQQQQQQQQQQQQQQQEQQQQQQQQQQQQ |
| 140 | G | 1 | #GGGGGGGGGGGGGGGGGGGGGGGGGGGGGGGGGGGGGGGGGGGGGGGGGGGGGGGGGGGGGGG |
| 141 | Q | 0.973 | #QQQQPQQQQQQQQQQQQQQQQQQQQQQQQQQQQQQQQQQQQQQQQQQQQQQQQQQQQQQQQQQ |
| 142 | M | 0.954 | #MMMMMMMMMMMMMMMMMMMMMMMMMMMMMMMIMMMMMMMMMIMMMMMMMMMMMMMMMMMMMMM |
| 143 | V | 1 | #VVVVVVVVVVVVVVVVVVVVVVVVVVVVVVVVVVVVVVVVVVVVVVVVVVVVVVVVVVVVVVV |
| 144 | H | 1 | #HHHHHHHHHHHHHHHHHHHHHHHHHHHHHHHHHHHHHHHHHHHHHHHHHHHHHHHHHHHHHHH |
| 145 | Q | 1 | #QQQQQQQQQQQQQQQQQQQQQQQQQQQQQQQQQQQQQQQQQQQQQQQQQQQQQQQQQQQQQQQ |
| 146 | A | 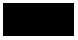 | #AAAAPAPAAASPAAAAAAAPPPPPPAAAPAAGPAPAPAAAAAAPAAAAAAAAAAAAAAAAAAA |
| 147 | I | 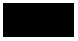 | #ILIIIIIIIIIIIIILIIIIIIIIIIIIIIILILIIIIILIIIILIIIIIIIIIIIILILLII |
| 148 | S | 1 | #SSSSSSSSSSSSSSSSSSSSSSSSSSSSSSSSSSSSSSSSSSSSSSSSSSSSSSSSSSSSSSS |

Table 1D

| | | | |
|---|---|---|---|
| 149 | P | 0.954 | #PPPPPPPPPPPPPPPPPAPPPPPPPPPPPPPPPPAPPPPPPPPPPPPPPPPPPPPPPPPPPPP |
| 150 | R | 1 | #RRRRRRRRRRRRRRRRRRRRRRRRRRRRRRRRRRRRRRRRRRRRRRRRRRRRRRRRRRRRRRR |
| 151 | T | 1 | #TTTTTTTTTTTTTTTTTTTTTTTTTTTTTTTTTTTTTTTTTTTTTTTTTTTTTTTTTTTTTTT |
| 152 | L | 0.937 | #LLLLLFLLLLLLLLLLLLLLLLLLLLLLLLLFLLFLLLLLLLLLLLLLLLLLLLLLLLLLLLL |
| 153 | N | 1 | #NNNNNNNNNNNNNNNNNNNNNNNNNNNNNNNNNNNNNNNNNNNNNNNNNNNNNNNNNNNNNNN |
| 154 | A | 1 | #AAAAAAAAAAAAAAAAAAAAAAAAAAAAAAAAAAAAAAAAAAAAAAAAAAAAAAAAAAAAAAA |
| 155 | W | 0.911 | #WWWWWWWWWWWWWWWWWWWWWWWWWWWWWWW$$WWWWWWWW$WRWWWWWWWWWWWWWWWWWWW |
| 156 | V | 1 | #VVVVVVVVVVVVVVVVVVVVVVVVVVVVVVVVVVVVVVVVVVVVVVVVVVVVVVVVVVVVVVV |
| 157 | K | 1 | #KKKKKKKKKKKKKKKKKKKKKKKKKKKKKKKKKKKKKKKKKKKKKKKKKKKKKKKKKKKKKKK |
| 158 | V | 1 | #VVVVVVVVVVVVVVVVVVVVVVVVVVVVVVVVVVVVVVVVVVVVVVVVVVVVVVVVVVVVVVV |
| 159 | V | 0.856 | #VVVVVVVVVVVVVVIVVVIVVVVVVVVVVVVIIVVVVIVIVVVIVVIVVVVVVVVVVVVIVVI |
| 160 | E | 1 | #EEEEEEEEEEEEEEEEEEEEEEEEEEEEEEEEEEEEEEEEEEEEEEEEEEEEEEEEEEEEEEE |
| 161 | E | 0.973 | #EEEEEEEEEEEEEEEEEEEEEEEEEEEEEEEEEEEEEEKEEEEEEEEEEEEEEEEEEEEEEEE |
| 162 | K | 1 | #KKKKKKKKKKKKKKKKKKKKKKKKKKKKKKKKKKKKKKKKKKKKKKKKKKKKKKKKKKKKKKK |
| 163 | A | 1 | #AAAAAAAAAAAAAAAAAAAAAAAAAAAAAAAAAAAAAAAAAAAAAAAAAAAAAAAAAAAAAAA |
| 164 | F | 1 | #FFFFFFFFFFFFFFFFFFFFFFFFFFFFFFFFFFFFFFFFFFFFFFFFFFFFFFFFFFFFFFF |
| 165 | S | 1 | #SSSSSSSSSSSSSSSSSSSSSSSSSSSSSSSSSSSSSSSSSSSSSSSSSSSSSSSSSSSSSSS |
| 166 | P | 1 | #PPPPPPPPPPPPPPPPPPPPPPPPPPPPPPPPPPPPPPPPPPPPPPPPPPPPPPPPPPPPPPP |
| 167 | E | 0.954 | #EEEEEEEEKEEEEEEEEEEEEEEEEEEEEEEEEEKEEEEEEEEEEEEEEEEEEEEEEEEEEEE |
| 168 | V | 0.92 | #VVMVVGVVVVVVVVVVVVVVVVVVVVVVVVVVIVVVVVVVVVVVVVVVVVVVVVVVVVVVVVV |
| 169 | I | 1 | #IIIIIIIIIIIIIIIIIIIIIIIIIIIIIIIIIIIIIIIIIIIIIIIIIIIIIIIIIIIIIII |
| 170 | P | 1 | #PPPPPPPPPPPPPPPPPPPPPPPPPPPPPPPPPPPPPPPPPPPPPPPPPPPPPPPPPPPPPPP |
| 171 | M | 1 | #MMMMMMMMMMMMMMMMMMMMMMMMMMMMMMMMMMMMMMMMMMMMMMMMMMMMMMMMMMMMMMM |
| 172 | F | 1 | #FFFFFFFFFFFFFFFFFFFFFFFFFFFFFFFFFFFFFFFFFFFFFFFFFFFFFFFFFFFFFFF |
| 173 | S | 0.821 | #SAAASSSSSSSSSSSSSTSSSSTTSSSSSSSSASSSTSSSSTSSSSSSSSSSSSSSSSSSSST |
| 174 | A | 1 | #AAAAAAAAAAAAAAAAAAAAAAAAAAAAAAAAAAAAAAAAAAAAAAAAAAAAAAAAAAAAAAA |
| 175 | L | 1 | #LLLLLLLLLLLLLLLLLLLLLLLLLLLLLLLLLLLLLLLLLLLLLLLLLLLLLLLLLLLLLLL |
| 176 | S | 0.922 | #SSSSASASSSSSSSSSSSSSSSSSSSSSASASSSSSSSSSSSSSSSSSSSSSSSSSSSSSSSS |
| 177 | E | 1 | #EEEEEEEEEEEEEEEEEEEEEEEEEEEEEEEEEEEEEEEEEEEEEEEEEEEEEEEEEEEEEEE |
| 178 | G | 1 | #GGGGGGGGGGGGGGGGGGGGGGGGGGGGGGGGGGGGGGGGGGGGGGGGGGGGGGGGGGGGGGG |
| 179 | A | 1 | #AAAAAAAAAAAAAAAAAAAAAAAAAAAAAAAAAAAAAAAAAAAAAAAAAAAAAAAAAAAAAAA |
| 180 | T | 0.973 | #TTTTTTTTTTITTTTTTTTTTTTTTTTTTTTTTTTTTTTTTTTTTTTTTTTTTTTTTTTTTTT |
| 181 | P | 1 | #PPPPPPPPPPPPPPPPPPPPPPPPPPPPPPPPPPPPPPPPPPPPPPPPPPPPPPPPPPPPPPP |
| 182 | Q | 1 | #QQQQQQQQQQQQQQQQQQQQQQQQQQQQQQQQQQQQQQQQQQQQQQQQQQQQQQQQQQQQQQQ |
| 183 | D | 1 | #DDDDDDDDDDDDDDDDDDDDDDDDDDDDDDDDDDDDDDDDDDDDDDDDDDDDDDDDDDDDDDD |
| 184 | L | 1 | #LLLLLLLLLLLLLLLLLLLLLLLLLLLLLLLLLLLLLLLLLLLLLLLLLLLLLLLLLLLLLLL |
| 185 | N | 0.973 | #NNNNNNNNNNNNNNNNNNNNNNNNNNNNNNNNNNNNNTNNNNNNNNNNNNNNNNNNNNNNNNN |
| 186 | T | 1 | #TTTTTTTTTTTTTTTTTTTTTTTTTTTTTTTTTTTTTTTTTTTTTTTTTTTTTTTTTTTTTTT |
| 187 | M | 1 | #MMMMMMMMMMMMMMMMMMMMMMMMMMMMMMMMMMMMMMMMMMMMMMMMMMMMMMMMMMMMMMM |
| 188 | L | 1 | #LLLLLLLLLLLLLLLLLLLLLLLLLLLLLLLLLLLLLLLLLLLLLLLLLLLLLLLLLLLLLLL |
| 189 | N | 1 | #NNNNNNNNNNNNNNNNNNNNNNNNNNNNNNNNNNNNNNNNNNNNNNNNNNNNNNNNNNNNNNN |
| 190 | T | 1 | #TTTTTTTTTTTTTTTTTTTTTTTTTTTTTTTTTTTTTTTTTTTTTTTTTTTTTTTTTTTTTTT |
| 191 | V | 0.947 | #VVGVVVXVVVVVVVVVVVVVVVVVVVVVVVVVVVVVVVVVVVVVVVVVVVVVVVVVVVVVVVV |
| 192 | G | 1 | #GGGGGGGGGGGGGGGGGGGGGGGGGGGGGGGGGGGGGGGGGGGGGGGGGGGGGGGGGGGGGGG |
| 193 | G | 0.973 | #GGAGGGGGGGGGGGGGGGGGGGGGGGGGGGGGGGGGGGGGGGGGGGGGGGGGGGGGGGGGGGG |
| 194 | H | 0.973 | #HHYHHHHHHHHHHHHHHHHHHHHHHHHHHHHHHHHHHHHHHHHHHHHHHHHHHHHHHHHHHHH |
| 195 | Q | 1 | #QQQQQQQQQQQQQQQQQQQQQQQQQQQQQQQQQQQQQQQQQQQQQQQQQQQQQQQQQQQQQQQ |
| 196 | A | 0.973 | #AAATAAAAAAAAAAAAAAAAAAAAAAAAAAAAAAAAAAAAAAAAAAAAAAAAAAAAAAAAAAA |
| 197 | A | 1 | #AAAAAAAAAAAAAAAAAAAAAAAAAAAAAAAAAAAAAAAAAAAAAAAAAAAAAAAAAAAAAAA |
| 198 | M | 1 | #MMMMMMMMMMMMMMMMMMMMMMMMMMMMMMMMMMMMMMMMMMMMMMMMMMMMMMMMMMMMMMM |
| 199 | Q | 0.973 | #QQQQQQQQQQQQQQQQQQQQQQQQQQQQQQQQQQQQQQQQQQQQQQQHQQQQQQQQQQQQQQQ |

Table 1E

| | | | |
|---|---|---|---|
| 200 | M | 1 | #MMMMMMMMMMMMMMMMMMMMMMMMMMMMMMMMMMMMMMMMMMMMMMMMMMMMMMMMMMMMMMM |
| 201 | L | 0.973 | #LLLFLLLLLLLLLLLLLLLLLLLLLLLLLLLLLLLLLLLLLLLLLLLLLLLLLLLLLLLLLLL |
| 202 | K | 1 | #KKKKKKKKKKKKKKKKKKKKKKKKKKKKKKKKKKKKKKKKKKKKKKKKKKKKKKKKKKKKKKK |
| 203 | E | 0.973 | #EEEEEEEEEEEEEEEEEEEEEEEEEEEEEEEEEEEEEEEEEEEEEEEEEEEEEEEEEEEEEED |
| 204 | T | 1 | #TTTTTTTTTTTTTTTTTTTTTTTTTTTTTTTTTTTTTTTTTTTTTTTTTTTTTTTTTTTTTTT |
| 205 | I | 1 | #IIIIIIIIIIIIIIIIIIIIIIIIIIIIIIIIIIIIIIIIIIIIIIIIIIIIIIIIIIIIIII |
| 206 | N | 1 | #NNNNNNNNNNNNNNNNNNNNNNNNNNNNNNNNNNNNNNNNNNNNNNNNNNNNNNNNNNNNNNN |
| 207 | E | 0.954 | #EEEEEEEEEEEEEEEEEEEEEEEEEEEEEEEDEEEEEDEEEEEEEEEEEEEEEEEEEEEEEEE |
| 208 | E | 1 | #EEEEEEEEEEEEEEEEEEEEEEEEEEEEEEEEEEEEEEEEEEEEEEEEEEEEEEEEEEEEEEE |
| 209 | A | 1 | #AAAAAAAAAAAAAAAAAAAAAAAAAAAAAAAAAAAAAAAAAAAAAAAAAAAAAAAAAAAAAAA |
| 210 | A | 0.973 | #AAAAAAAAAAAAAAAGAAAAAAAAAAAAAAAAAAAAAAAAAAAAAAAAAAAAAAAAAAAAAAA |
| 211 | E | 0.881 | #EEEEEEEEEEEDEEEEEEEEEEKEEEEEEEEEKEEEE$EEEEEDEEEEEEEEEEEEEEEEEEE |
| 212 | W | 0.937 | #WWWWWWWWWWWWWWWWWWWWWWWWWWWWWWW$$WWWWWWWW$WWWWWWWWWWWWWWWWWWWWW |
| 213 | D | 1 | #DDDDDDDDDDDDDDDDDDDDDDDDDDDDDDDDDDDDDDDDDDDDDDDDDDDDDDDDDDDDDDD |
| 214 | R | 0.973 | #RRRRRRRRRRRRRRRRRRRRRRRRRRRRRRRRRRRRRRRRRKRRRRRRRRRRRRRRRRRRRRR |
| 215 | L | 0.909 | #LLLLLLLLLLLLLVLLLLLLLLLLLLLVLLVLLLLLLLLLLLLLLLVLLVLLLLLLLLLLLLL |
| 216 | H | 1 | #HHHHHHHHHHHHHHHHHHHHHHHHHHHHHHHHHHHHHHHHHHHHHHHHHHHHHHHHHHHHHHH |
| 217 | P | 1 | #PPPPPPPPPPPPPPPPPPPPPPPPPPPPPPPPPPPPPPPPPPPPPPPPPPPPPPPPPPPPPPP |
| 218 | V | 0.909 | #VVVVVVVVVVVVVVAAVVAVVVVVVVVVVVVVVAVVVVVVVVAVVVVVVVVVVVVVVVVVVVV |
| 219 | H | 0.801 | #HHQHHHHHHHHHHHQQHQHHHHHHHHHHHHHHQQHHQPHQQHQHHHQHHHHHHHHHQQQHHHH |
| 220 | A | 1 | #AAAAAAAAAAAAAAAAAAAAAAAAAAAAAAAAAAAAAAAAAAAAAAAAAAAAAAAAAAAAAAA |
| 221 | G | 0.973 | #GGGGGGGGGGGGGGGGGGGGGGGGGGGGGGGGRGGGGGGGGGGGGGGGGGGGGGGGGGGGGGG |
| 222 | P | 0.973 | #PPPPPPPPPPPPPPPPPPPPPPPPPPPPPPPPPPPPPPPPPPPPPPLPPPPPPPPPPPPPPPP |
| 223 | I | 0.739 | #IIVIIIIIIIVIIIVIIVNVIIIIIIHIIIVVIVVINIIHVIIIIIIIIIIIIIIIVVVIIIV |
| 224 | A | 0.911 | #AAAAAAAAAAAAAAAAAAAAAAAAAAPAAAAAAAAAPAAPAAAAAAAAAATAAAAAAAAAAAA |
| 225 | P | 1 | #PPPPPPPPPPPPPPPPPPPPPPPPPPPPPPPPPPPPPPPPPPPPPPPPPPPPPPPPPPPPPPP |
| 226 | G | 1 | #GGGGGGGGGGGGGGGGGGGGGGGGGGGGGGGGGGGGGGGGGGGGGGGGGGGGGGGGGGGGGGG |
| 227 | Q | 1 | #QQQQQQQQQQQQQQQQQQQQQQQQQQQQQQQQQQQQQQQQQQQQQQQQQQQQQQQQQQQQQQQ |
| 228 | M | 0.908 | #MMIMMMMMMMMMMMMMMMMMMMMMMMMMMMMMMLMMLMMMMIMMMMMMMMMMMMMMMMMMMMM |
| 229 | R | 0.973 | #RRRRRRRRRRRRRRRRRKRRRRRRRRRRRRRRRRRRRRRRRRRRRRRRRRRRRRRRRRRRRRR |
| 230 | E | 0.937 | #EEEEEEEEEEEEEEDEEEEEEEEEEEEEEEEEEDEEEDEEEEEEEEEEEEEEEEEEEEEEEEE |
| 231 | P | 1 | #PPPPPPPPPPPPPPPPPPPPPPPPPPPPPPPPPPPPPPPPPPPPPPPPPPPPPPPPPPPPPPP |
| 232 | R | 0.973 | #RRRRRRRRRRRRRRRRRRRRRTRRRRRRRRRRRRRRRRRRRRRRRRRRRRRRRRRRRRRRRRR |
| 233 | G | 0.954 | #GGGGGGGGGGGGGGGGGGGGGGGGGGGGGGGRGGGGGGGGGRGGGGGGGGGGGGGGGGGGGGG |
| 234 | S | 1 | #SSSSSSSSSSSSSSSSSSSSSSSSSSSSSSSSSSSSSSSSSSSSSSSSSSSSSSSSSSSSSSS |
| 235 | D | 1 | #DDDDDDDDDDDDDDDDDDDDDDDDDDDDDDDDDDDDDDDDDDDDDDDDDDDDDDDDDDDDDDD |
| 236 | I | 1 | #IIIIIIIIIIIIIIIIIIIIIIIIIIIIIIIIIIIIIIIIIIIIIIIIIIIIIIIIIIIIIII |
| 237 | A | 1 | #AAAAAAAAAAAAAAAAAAAAAAAAAAAAAAAAAAAAAAAAAAAAAAAAAAAAAAAAAAAAAAA |
| 238 | G | 0.973 | #GGGGGGRGGGGGGGGGGGGGGGGGGGGGGGGGGGGGGGGGGGGGGGGGGGGGGGGGGGGGGGG |
| 239 | T | 0.909 | #TTTTSSSSTTTTTTTTTTTTTTTTTTTTTTTTTTTTTTTTSTTTTTTTTTTTTTTTTTTTTTT |
| 240 | T | 1 | #TTTTTTTTTTTTTTTTTTTTTTTTTTTTTTTTTTTTTTTTTTTTTTTTTTTTTTTTTTTTTTT |
| 241 | S | 1 | #SSSSSSSSSSSSSSSSSSSSSSSSSSSSSSSSSSSSSSSSSSSSSSSSSSSSSSSSSSSSSSS |
| 242 | T | 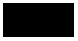 | #TTTTTTTTTTTTTTTTTTTTTTTTTTTTTTTTNTTTTTTTTTTTTTTTTTTTTTTTTTTTTTT |
| 243 | L | 1 | #LLLLLLLLLLLLLLLLLLLLLLLLLLLLLLLLLLLLLLLLLLLLLLLLLLLLLLLLLLLLLLL |
| 244 | Q | 1 | #QQQQQQQQQQQQQQQQQQQQQQQQQQQQQQQQQQQQQQQQQQQQQQQQQQQQQQQQQQQQQQQ |
| 245 | E | 1 | #EEEEEEEEEEEEEEEEEEEEEEEEEEEEEEEEEEEEEEEEEEEEEEEEEEEEEEEEEEEEEEE |
| 246 | Q | 0.973 | #QQQQQQQQQQQQQQQQHQQQQQQQQQQQQQQQQQQQQQQQQQQQQQQQQQQQQQQQQQQQQQQ |
| 247 | I | 1 | #IIIIIIIIIIIIIIIIIIIIIIIIIIIIIIIIIIIIIIIIIIIIIIIIIIIIIIIIIIIIIII |
| 248 | G | 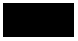 | #GGGGGNGNGGGGGGGGGGGGGGGGGGAGGGGGAGNGGGGGRGGGGGGGGGGGGGGGGGGAGGA |
| 249 | W | 0.973 | #WWWWWWWWWWWWWWWWWWWWWWWWWWWWWWW$WWWWWWWWWWWWWWWWWWWWWWWWWWWWWWW |
| 250 | M | 0.973 | #MMMMMMMMMMMMMMMMMMMMMMMMMMMMMMMMMMMMMMMMMMMMMMMMMMMMMMMMMMMMMMI |

Table 1F

| | | | |
|---|---|---|---|
| 251 | T | 1 | #TTTTTTTTTTTTTTTTTTTTTTTTTTTTTTTTTTTTTTTTTTTTTTTTTTTTTTTTTTTTTTT |
| 252 | N | 0.625 | #NSNSQSHSNNNNNNGSNNSNNHHHNNNNNNNNNHGHNSNHSNSNHNSHNNNHNNNNNHNNNNA |
| 253 | N | 0.937 | #NTNTTNNNNNNNNNNNNNNNNNNNNNNNNNNNNNNNNNNNNNNNNNNNNNNNNNNNNNNNNNN |
| 254 | P | 1 | #PPPPPPPPPPPPPPPPPPPPPPPPPPPPPPPPPPPPPPPPPPPPPPPPPPPPPPPPPPPPPPP |
| 255 | P | 0.954 | #PPPPPPPPPPPPPPAPPPPPPPPPPPPPPPPPPPAPPPPPPPPPPPPPPPPPPPPPPPPPPPP |
| 256 | I | 0.891 | #ITITIIIIIIIIIIIIIIVIIIVVIIIIIIIIIIIIIIIIIIIIIIIIIIIIIIIIIIIIIII |
| 257 | P | 1 | #PPPPPPPPPPPPPPPPPPPPPPPPPPPPPPPPPPPPPPPPPPPPPPPPPPPPPPPPPPPPPPP |
| 258 | V | 1 | #VVVVVVVVVVVVVVVVVVVVVVVVVVVVVVVVVVVVVVVVVVVVVVVVVVVVVVVVVVVVVVV |
| 259 | G | 0.973 | #GGGGGGGGGGGGGGGGGGGGGGGGGGGGGGGGGGGGGGGGGRGGGGGGGGGGGGGGGGGGGGG |
| 260 | E | 0.859 | #EEEEDEDEEEEEEEEEDDEEEDEEEEEEEEEEDEEDEEEEEKEEEEEEEEEEEEEEEEEEEEE |
| 261 | I | 1 | #IIIIIIIIIIIIIIIIIIIIIIIIIIIIIIIIIIIIIIIIIIIIIIIIIIIIIIIIIIIIIII |
| 262 | Y | 1 | #YYYYYYYYYYYYYYYYYYYYYYYYYYYYYYYYYYYYYYYYYYYYYYYYYYYYYYYYYYYYYYY |
| 263 | K | 1 | #KKKKKKKKKKKKKKKKKKKKKKKKKKKKKKKKKKKKKKKKKKKKKKKKKKKKKKKKKKKKKKK |
| 264 | R | 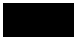 | #RRRRRRRRRRRRRRRRRRRRRRRRRRRRRRRRKRRRRRRRRRRRRRRRRRRRRRRRRRRRRRR |
| 265 | W | 0.954 | #WWWWWWWWWWWWWWWWWWWWWWWWWWWWWWWW$WWWWWWWW$WWWWWWWWWWWWWWWWWWWWW |
| 266 | I | 1 | #IIIIIIIIIIIIIIIIIIIIIIIIIIIIIIIIIIIIIIIIIIIIIIIIIIIIIIIIIIIIIII |
| 267 | I | 1 | #IIIIIIIIIIIIIIIIIIIIIIIIIIIIIIIIIIIIIIIIIIIIIIIIIIIIIIIIIIIIIII |
| 268 | L | 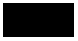 | #LLLLLLLLLLLLLLLLLLLLLLLLLLLLLLLLLLLLLLLLLLLLLLLLLLLLLLLLLMLLMLL |
| 269 | G | 1 | #GGGGGGGGGGGGGGGGGGGGGGGGGGGGGGGGGGGGGGGGGGGGGGGGGGGGGGGGGGGGGGG |
| 270 | L | 1 | #LLLLLLLLLLLLLLLLLLLLLLLLLLLLLLLLLLLLLLLLLLLLLLLLLLLLLLLLLLLLLLL |
| 271 | N | 1 | #NNNNNNNNNNNNNNNNNNNNNNNNNNNNNNNNNNNNNNNNNNNNNNNNNNNNNNNNNNNNNNN |
| 272 | K | 0.973 | #KKKKKKKKKKKKKKKKIKKKKKKKKKKKKKKKKKKKKKKKKKKKKKKKKKKKKKKKKKKKKKK |
| 273 | I | 1 | #IIIIIIIIIIIIIIIIIIIIIIIIIIIIIIIIIIIIIIIIIIIIIIIIIIIIIIIIIIIIIII |
| 274 | V | 1 | #VVVVVVVVVVVVVVVVVVVVVVVVVVVVVVVVVVVVVVVVVVVVVVVVVVVVVVVVVVVVVVV |
| 275 | R | 0.954 | #RRRRRRRRRRRRRRRRRRRRRRRRRRRRRRRKKRRRRRRRRRRRRRRRRRRRRRRRRRRRRRR |
| 276 | M | 1 | #MMMMMMMMMMMMMMMMMMMMMMMMMMMMMMMMMMMMMMMMMMMMMMMMMMMMMMMMMMMMMMM |
| 277 | Y | 1 | #YYYYYYYYYYYYYYYYYYYYYYYYYYYYYYYYYYYYYYYYYYYYYYYYYYYYYYYYYYYYYYY |
| 278 | S | 1 | #SSSSSSSSSSSSSSSSSSSSSSSSSSSSSSSSSSSSSSSSSSSSSSSSSSSSSSSSSSSSSSS |
| 279 | P | 1 | #PPPPPPPPPPPPPPPPPPPPPPPPPPPPPPPPPPPPPPPPPPPPPPPPPPPPPPPPPPPPPPP |
| 280 | T | 0.633 | #TTATTTTTTTTTTTITTTVTTSTTTTTTTTVVTSIAVTSVTASATTITITSTTTTTSTSVTTV |
| 281 | S | 0.927 | #SSSSSSSSSSSSSSSSSSSCSSSSSSSSSSSSSSSSGSSCSSSSSSSSSSSSSSSSSSSSSSS |
| 282 | I | 1 | #IIIIIIIIIIIIIIIIIIIIIIIIIIIIIIIIIIIIIIIIIIIIIIIIIIIIIIIIIIIIIII |
| 283 | L | 1 | #LLLLLLLLLLLLLLLLLLLLLLLLLLLLLLLLLLLLLLLLLLLLLLLLLLLLLLLLLLLLLLL |
| 284 | D | 1 | #DDDDDDDDDDDDDDDDDDDDDDDDDDDDDDDDDDDDDDDDDDDDDDDDDDDDDDDDDDDDDDD |
| 285 | I | 1 | #IIIIIIIIIIIIIIIIIIIIIIIIIIIIIIIIIIIIIIIIIIIIIIIIIIIIIIIIIIIIIII |
| 286 | R | 0.865 | #RRRRKRKRRRRKRRKRRRRRRRRRRKKRRRRRRKRRRRRKRRRRRRRRRRRRRRRRRRKRRRR |
| 287 | Q | 1 | #QQQQQQQQQQQQQQQQQQQQQQQQQQQQQQQQQQQQQQQQQQQQQQQQQQQQQQQQQQQQQQQ |
| 288 | G | 0.954 | #GGGGGGGGGGGGGGGGGGGGGGGGGGGGGGGRRGGGGGGGGGGGGGGGGGGGGGGGGGGGGGG |
| 289 | P | 1 | #PPPPPPPPPPPPPPPPPPPPPPPPPPPPPPPPPPPPPPPPPPPPPPPPPPPPPPPPPPPPPPP |
| 290 | K | 1 | #KKKKKKKKKKKKKKKKKKKKKKKKKKKKKKKKKKKKKKKKKKKKKKKKKKKKKKKKKKKKKKK |
| 291 | E | 1 | #EEEEEEEEEEEEEEEEEEEEEEEEEEEEEEEEEEEEEEEEEEEEEEEEEEEEEEEEEEEEEEE |
| 292 | P | 0.937 | #PPPPPPPPPPPPPPPSPPPPPPPPPPPPPPPPPPPPPPPPPPPPSPPPPPPPPPPPSPPPPPP |
| 293 | F | 1 | #FFFFFFFFFFFFFFFFFFFFFFFFFFFFFFFFFFFFFFFFFFFFFFFFFFFFFFFFFFFFFFF |
| 294 | R | 0.954 | #RRRRRRRRRRRRRRRRRRRRRRRRRRRRRRRKRRRRRRRRRKRRRRRRRRRRRRRRRRRRRRR |
| 295 | D | 1 | #DDDDDDDDDDDDDDDDDDDDDDDDDDDDDDDDDDDDDDDDDDDDDDDDDDDDDDDDDDDDDDD |
| 296 | Y | 1 | #YYYYYYYYYYYYYYYYYYYYYYYYYYYYYYYYYYYYYYYYYYYYYYYYYYYYYYYYYYYYYYY |
| 297 | V | 1 | #VVVVVVVVVVVVVVVVVVVVVVVVVVVVVVVVVVVVVVVVVVVVVVVVVVVVVVVVVVVVVVV |
| 298 | D | 0.973 | #DDDDDEDDDDDDDDDDDDDDDDDDDDDDDDDDDDDDDDDDDDDDDDDDDDDDDDDDDDDDDDD |
| 299 | R | 0.954 | #RRRRRRRRRRRRRRRRRRRRRRRRRRRRRRRRQRRRRRRRRQRRRRRRRRRRRRRRRRRRRRR |
| 300 | F | 1 | #FFFFFFFFFFFFFFFFFFFFFFFFFFFFFFFFFFFFFFFFFFFFFFFFFFFFFFFFFFFFFFF |
| 301 | Y | 0.947 | #YYYYYYNYYYYYYYYYYYYYYYYYYYYYYYYYYYYYYYYYYYYYYYYYYYYYYYYYYYYYYYF |

Table 1G

| | | | |
|---|---|---|---|
| 302 | K | 0.954 | #KKKKKKKKKKKRKKKKKKKKKKKKKKKKKKKKKRKKKKKKKKKKKKKKKKKKKKKKKKKKKKK |
| 303 | T | 0.954 | #TTTTVTVTTTTTTTTTTTTTTTTTTTTTTTTTTTTTTTTTTTTTTTTTTTTTTTTTTTTTTTT |
| 304 | L | 1 | #LLLLLLLLLLLLLLLLLLLLLLLLLLLLLLLLLLLLLLLLLLLLLLLLLLLLLLLLLLLLLLL |
| 305 | R | 1 | #RRRRRRRRRRRRRRRRRRRRRRRRRRRRRRRRRRRRRRRRRRRRRRRRRRRRRRRRRRRRRRR |
| 306 | A | 0.973 | #AAAAAAAAAAAAAAAAAAAAAAAAAAAAAAAAAAAAAAAAAAAAATAAAAAAAAAAAAAAAAA |
| 307 | E | 1 | #EEEEEEEEEEEEEEEEEEEEEEEEEEEEEEEEEEEEEEEEEEEEEEEEEEEEEEEEEEEEEEE |
| 308 | Q | 1 | #QQQQQQQQQQQQQQQQQQQQQQQQQQQQQQQQQQQQQQQQQQQQQQQQQQQQQQQQQQQQQQQ |
| 309 | A | 0.954 | #AAAACACAAAAAAAAAAAAAAAAAAAAAAAAAAAAAAAAAAAAAAAAAAAAAAAAAAAAAAAA |
| 310 | S | 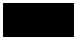 | #SSSSTSTSSSSSSSTSSSSSSSSSTTSSSSSSSSSSSSSSSSSSSSSSSSSSSSSSSSSTSST |
| 311 | Q | 1 | #QQQQQQQQQQQQQQQQQQQQQQQQQQQQQQQQQQQQQQQQQQQQQQQQQQQQQQQQQQQQQQQ |
| 312 | E | 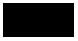 | #EDEDDEDEEEEEEDEEEEDDDDDDEEEEDEDEEEDDEEEEEEEEEDEEDDEEEEEEEEEEEED |
| 313 | V | 1 | #VVVVVVVVVVVVVVVVVVVVVVVVVVVVVVVVVVVVVVVVVVVVVVVVVVVVVVVVVVVVVVV |
| 314 | K | 1 | #KKKKKKKKKKKKKKKKKKKKKKKKKKKKKKKKKKKKKKKKKKKKKKKKKKKKKKKKKKKKKKK |
| 315 | N | 1 | #NNNNNNNNNNNNNNNNNNNNNNNNNNNNNNNNNNNNNNNNNNNNNNNNNNNNNNNNNNNNNNN |
| 316 | W | 0.911 | #WWWWWWWWWWWWWWWWWWWWWWWWWWWWWWW$$WWWWWWWW$WWWWWWWWRWWWWWWWWWWWW |
| 317 | M | 0.973 | #MMMMMMMMMMMMMMMMMMMMMMMMMMMMMMMMMMMMMMMMMMMMMMMMMMTMMMMMMMMMMMM |
| 318 | T | 1 | #TTTTTTTTTTTTTTTTTTTTTTTTTTTTTTTTTTTTTTTTTTTTTTTTTTTTTTTTTTTTTTT |
| 319 | E | 0.954 | #EEEEEEEEEEEEEEEEEEEEEEEEEEEEEEEEEEEEEEEEEDEEEEEEEEEEEEEEEEEEEED |
| 320 | T | 1 | #TTTTTTTTTTTTTTTTTTTTTTTTTTTTTTTTTTTTTTTTTTTTTTTTTTTTTTTTTTTTTTT |
| 321 | L | 0.973 | #LLLLLLLLLLLLLLLLLLLLLLLLLLLLLLLLLLLLLLLLLLLLLLLLFLLLLLLLLLLLLLL |
| 322 | L | 1 | #LLLLLLLLLLLLLLLLLLLLLLLLLLLLLLLLLLLLLLLLLLLLLLLLLLLLLLLLLLLLLLL |
| 323 | V | 1 | #VVVVVVVVVVVVVVVVVVVVVVVVVVVVVVVVVVVVVVVVVVVVVVVVVVVVVVVVVVVVVVV |
| 324 | Q | 0.973 | #QQQQQQQQQQQQQQQQQQQQQQQQQQQQQQQQQQHQQQQQQQQQQQQQQQQQQQQQQQQQQQQ |
| 325 | N | 0.973 | #NNNNNKNNNNNNNNNNNNNNNNNNNNNNNNNNNNNNNNNNNNNNNNNNNNNNNNNNNNNNNNN |
| 326 | A | 0.896 | #AAAAPAAAAAAAAAASAAAAAAAAAAAAAAAAAAAASAAAAAAAAAAAAAAASAAAAAAASAA |
| 327 | N | 1 | #NNNNNNNNNNNNNNNNNNNNNNNNNNNNNNNNNNNNNNNNNNNNNNNNNNNNNNNNNNNNNNN |
| 328 | P | 1 | #PPPPPPPPPPPPPPPPPPPPPPPPPPPPPPPPPPPPPPPPPPPPPPPPPPPPPPPPPPPPPPP |
| 329 | D | 0.973 | #DDDDDDDDDDDDDDDDDDDDDDDDDDDDDDDDDDHDDDDDDDDDDDDDDDDDDDDDDDDDDDD |
| 330 | C | 1 | #CCCCCCCCCCCCCCCCCCCCCCCCCCCCCCCCCCCCCCCCCCCCCCCCCCCCCCCCCCCCCCC |
| 331 | K | 0.896 | #KKKKKKKKKKKRKKKKKRKKKKRRKKKKKKKKKKKKKKKKKKKKKKKEKKKKKKKKKKKKKKK |
| 332 | T | 0.973 | #TTTTTTTTTTTTTTTTTTTTTTTTTTTTTTTTTTTTNTTTTTTTTTTTTTTTTTTTTTTTTTT |
| 333 | I | 0.973 | #IIIIIIFIIIIIIIIIIIIIIIIIIIIIIIIIIIIIIIIIIIIIIIIIIIIIIIIIIIIIIII |
| 334 | L | 1 | #LLLLLLLLLLLLLLLLLLLLLLLLLLLLLLLLLLLLLLLLLLLLLLLLLLLLLLLLLLLLLLL |
| 335 | K | 0.973 | #KKKKKKKKKKKKKKKKKKKKKKKKKKKKKKKKKKKKKKKKKKKKKKKKKKKKKKKKKKKKKKR |
| 336 | A | 1 | #AAAAAAAAAAAAAAAAAAAAAAAAAAAAAAAAAAAAAAAAAAAAAAAAAAAAAAAAAAAAAAA |
| 337 | L | 0.973 | #LLLLFLLLLLLLLLLLLLLLLLLLLLLLLLLLLLLLLLLLLLLLLLLLLLLLLLLLLLLLLLL |
| 338 | G | 0.973 | #GGGGRGGGGGGGGGGGGGGGGGGGGGGGGGGGGGGGGGGGGGGGGGGGGGGGGGGGGGGGGGG |
| 339 | P | 1 | #PPPPPPPPPPPPPPPPPPPPPPPPPPPPPPPPPPPPPPPPPPPPPPPPPPPPPPPPPPPPPPP |
| 340 | A | 0.84 | #AAAAAAAAAAAAGAAAAAAAAAAAAAAAAAAAAAAAAAAAAGAGGAGGAAAAAAAAGGGGGAG |
| 341 | A | 1 | #AAAAAAAAAAAAAAAAAAAAAAAAAAAAAAAAAAAAAAAAAAAAAAAAAAAAAAAAAAAAAAA |
| 342 | T | 1 | #TTTTTTTTTTTTTTTTTTTTTTTTTTTTTTTTTTTTTTTTTTTTTTTTTTTTTTTTTTTTTTT |
| 343 | L | 1 | #LLLLLLLLLLLLLLLLLLLLLLLLLLLLLLLLLLLLLLLLLLLLLLLLLLLLLLLLLLLLLLL |
| 344 | E | 0.973 | #EEEEEEEEEEEEEEEEEKEEEEEEEEEEEEEEEEEEEEEEEEEEEEEEEEEEEEEEEEEEEEE |
| 345 | E | 0.927 | #EEEEEEEEEEEEEEEEEEEEEEEEEEEEEEEKEEEEEEEEDEEEEEEEEEEEEEEEEEEDEEE |
| 346 | M | 1 | #MMMMMMMMMMMMMMMMMMMMMMMMMMMMMMMMMMMMMMMMMMMMMMMMMMMMMMMMMMMMMMM |
| 347 | M | 1 | #MMMMMMMMMMMMMMMMMMMMMMMMMMMMMMMMMMMMMMMMMMMMMMMMMMMMMMMMMMMMMMM |
| 348 | T | 0.973 | #TTTPTTTTTTTTTTTTTTTTTTTTTTTTTTTTTTTTTTTTTTTTTTTTTTTTTTTTTTTTTTT |
| 349 | A | 1 | #AAAAAAAAAAAAAAAAAAAAAAAAAAAAAAAAAAAAAAAAAAAAAAAAAAAAAAAAAAAAAAA |
| 350 | C | 1 | #CCCCCCCCCCCCCCCCCCCCCCCCCCCCCCCCCCCCCCCCCCCCCCCCCCCCCCCCCCCCCCC |
| 351 | Q | 1 | #QQQQQQQQQQQQQQQQQQQQQQQQQQQQQQQQQQQQQQQQQQQQQQQQQQQQQQQQQQQQQQQ |
| 352 | G | 0.973 | #GGGGEGGGGGGGGGGGGGGGGGGGGGGGGGGGGGGGGGGGGGGGGGGGGGGGGGGGGGGGGGG |

Table 1H

| | | | |
|---|---|---|---|
| 353 | V | 0.973 | #VVVVVVVVVVVVVVVVVVVVVVVVVVVVVVVVVVGVVVVVVVVVVVVVVVVVVVVVVVVVVVV |
| 354 | G | 0.973 | #GGGGGGGGGGGGGGGGGGGGGGGGGGGGGGGGGGGGGGGGGEGGGGGGGGGGGGGGGGGGGGG |
| 355 | 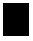 | 1 | #GGGGGGGGGGGGGGGGGGGGGGGGGGGGGGGGGGGGGGGGGGGGGGGGGGGGGGGGGGGGGGG |
| 356 | 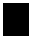 | 1 | #PPPPPPPPPPPPPPPPPPPPPPPPPPPPPPPPPPPPPPPPPPPPPPPPPPPPPPPPPPPPPPP |
| 357 | 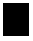 | 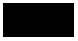 | #GSGGSGHSGGSGGGSGGGSSSSSSGGGGGGGGGAGAGGGSGAGSGSGGSGGGSGGGGGGGGGS |
| 358 | 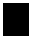 | 1 | #HHHHHHHHHHHHHHHHHHHHHHHHHHHHHHHHHHHHHHHHHHHHHHHHHHHHHHHHHHHHHHH |
| 359 | 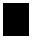 | 1 | #KKKKKKKKKKKKKKKKKKKKKKKKKKKKKKKKKKKKKKKKKKKKKKKKKKKKKKKKKKKKKKK |
| 360 | 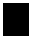 | 1 | #AAAAAAAAAAAAAAAAAAAAAAAAAAAAAAAAAAAAAAAAAAAAAAAAAAAAAAAAAAAAAAA |
| 361 | 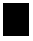 | 0.973 | #RRRRRRRRRRRRRRRRRRRRRRRKRRRRRRRRRRRRRRRRRRRRRRRRRRRRRRRRRRRRRRR |
| 362 | 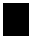 | 0.885 | #VIVVVVVVVVIVVVVVVVIIVVIIVVVVVVVVVVVVVVVVVVVVVVVVIVVVVVVVVVVVVVV |
| 363 | 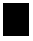 | 1 | #LLLLLLLLLLLLLLLLLLLLLLLLLLLLLLLLLLLLLLLLLLLLLLLLLLLLLLLLLLLLLLL |
| 364 | A | 1 | #AAAAAAAAAAAAAAAAAAAAAAAAAAAAAAAAAAAAAAAAAAAAAAAAAAAAAAAAAAAAAAA |
| 365 | E | 1 | #EEEEEEEEEEEEEEEEEEEEEEEEEEEEEEEEEEEEEEEEEEEEEEEEEEEEEEEEEEEEEEE |
| 366 | A | 1 | #AAAAAAAAAAAAAAAAAAAAAAAAAAAAAAAAAAAAAAAAAAAAAAAAAAAAAAAAAAAAAAA |
| 367 | M | 0.973 | #MMMMMMMMMMMMMMMMMMMMMMMMMMMMMMMIMMMMMMMMMMMMMMMMMMMMMMMMMMMMMMM |
| 368 | S | 1 | #SSSSSSSSSSSSSSSSSSSSSSSSSSSSSSSSSSSSSSSSSSSSSSSSSSSSSSSSSSSSSSS |
| 369 | Q | 0.947 | #QQQQQQQQQQQQQQQQQQQQHQQQQQQQQQQQQQQQQQQQQQQQQRQQQQQQQQQQQQQQQQQ |
| 370 | V | 0.694 | #VAMMAV--PVVVVVAVAAVVMAVVVVVVVVVVAVVVVVVAVVVAVVVVVVVVAVVVVVVVIVA |
| 371 | T | 0.818 | #TTTTS---TTTTTTTTTTTTTTTTTTTT-T-TTTTTTTTT-TTTTPTTTTTTTTTTTTTTTTN |
| 372 | N | 0.795 | #NNNNNNSSNNNNNNNNQSSNNNNNNGNNNNNNSNNNNSSGNNNNNNNNNNNNSNNNNSNNNNN |
| 373 | S | 0.704 | #SSSSSPSSASSSSSASSSPSSSPPSSSSSSSPSSSSPSSGTPSSSSSPSPSPSSPSSSSTTSA |
| 374 | A | 0.733 | #ANAASAAAAAAAAAAAAAAAAHAAAAAAVAAAATNTAAAATATAATAAAAAAAAAAAAAASAV |
| 375 | T | 0.651 | #TITTVSPTTATTTTTTTANIAATTTATTTTTTATVTTTTTTTTTTNATTNTTTTTTTTTTAT- |
| 376 | I | 0.825 | #IIIIMIIIIIIIIIVTVVIIVIIIVIIIVIIIIIIIIIIIIIIIIIIIIIIIIIIIIIIMIII |
| 377 | M | 1 | #MMMMMMMMMMMMMMMMMMMMMMMMMMMMMMMMMMMMMMMMMMMMMMMMMMMMMMMMMMMMMMM |
| 378 | M | 0.911 | #MMMMMIMMMMMMMMMMMMMMMMMMMMMMMMMMMMMMMMMMMMMMMMMILMMIMMMMMMMMMMM |
| 379 | Q | 1 | #QQQQQQQQQQQQQQQQQQQQQQQQQQQQQQQQQQQQQQQQQQQQQQQQQQQQQQQQQQQQQQQ |
| 380 | R | 0.801 | #RRRRRKRRRKRRRRRRRRRRRRRRRRKRKRKKRRRRRRRKKKRRRRRKKRRRRRRRRKRRGKK |
| 381 | G | 0.883 | #GGGGGGGGGGGGGGSSSSGGGGGGGGGGGGGGGGGGGDGGGGGGGGGGGGGGGGGGGGGGGGS |
| 382 | N | 0.947 | #NNNNNNINNNNNNNNNNNNNNNNNNNNNNNNNNNNNNKNNNNNNNNNNNNNNNNNNNNNNNNN |
| 383 | F | 0.927 | #FFFFFFFFFFFFFFFFFFFFFFFFFFFFFFFFFFFFFFLYFFFFFYFFFFFFFFFFFFFFFFF |
| 384 | R | 0.859 | #RRSKRRRRRRRRRRRRRRRRRRRRRRRRKRRKRRRRRRRRKKRRRRKRRRRRRRRRRRRRRRK |
| 385 | N | 0.851 | #NNNNSNNNNNNNNNNSNNNNNNNNNNNNNNSNGNNNNNNNNNNNNNNNDNNNNNNNNNNSNNG |
| 386 | Q | 0.911 | #QQQSQQQQQQQQQQQQQQQQQQQQQQQQQQQQQQQQPQQQQQQQQQQQQQQQQQQQQQQPQQP |
| 387 | R | 0.937 | #RRRRRRRRRRRRRRRRKKRRRRRRRRRRRRRRRRRRRRRRRRRRRRRRRRRKRRRRRRRRRRR |
| 388 | K | 0.897 | #KRKRKKRKKKKKKKKKKKKKRKKKKKKKKKKKKKKKKKKKKKKRKKKKKKKKKRKKKKKKKKK |
| 389 | T | 0.585 | #TIPIPPITTTTTTTNNNNTITIPTTTIITTTTIITIIIITTAITITMTITITITITTTTT-II |
| 390 | V | 0.885 | #VVVVVVVVVVVVVVVVIVIVVVVVVVVVVVVVVVVVVVVVVVIVVVVVVVIVVVVVVVVIIVI |
| 391 | K | 0.973 | #KKKKKKKKKKKKKKKKKKKKKKKKKKKKKKKKKKKKKKKKKKKKKKKKKKKKKKKKKKKKKKR |
| 392 | C | 1 | #CCCCCCCCCCCCCCCCCCCCCCCCCCCCCCCCCCCCCCCCCCCCCCCCCCCCCCCCCCCCCCC |
| 393 | F | 1 | #FFFFFFFFFFFFFFFFFFFFFFFFFFFFFFFFFFFFFFFFFFFFFFFFFFFFFFFFFFFFFFF |
| 394 | N | 1 | #NNNNNNNNNNNNNNNNNNNNNNNNNNNNNNNNNNNNNNNNNNNNNNNNNNNNNNNNNNNNNNN |
| 395 | C | 1 | #CCCCCCCCCCCCCCCCCCCCCCCCCCCCCCCCCCCCCCCCCCCCCCCCCCCCCCCCCCCCCCC |
| 396 | G | 1 | #GGGGGGGGGGGGGGGGGGGGGGGGGGGGGGGGGGGGGGGGGGGGGGGGGGGGGGGGGGGGGGG |
| 397 | K | 1 | #KKKKKKKKKKKKKKKKKKKKKKKKKKKKKKKKKKKKKKKKKKKKKKKKKKKKKKKKKKKKKKK |
| 398 | E | 0.954 | #EEEEEEEEEEEEEEEEEEEEEEEEEEEEEVEEEEEEEEEEEEEEEEEEVEEEEEEEEEEEEEE |
| 399 | G | 0.937 | #GGGGGGGGGGGGGGGGGGGGGGGGGGGGGGGRGGGGGGGGRRGGGGGGGGGGGGGGGGGGGGG |
| 400 | H | 1 | #HHHHHHHHHHHHHHHHHHHHHHHHHHHHHHHHHHHHHHHHHHHHHHHHHHHHHHHHHHHHHHH |
| 401 | I | 0.839 | #IIII-VIIIIIIIIIIIILIIIIIIIITIIIIIILIIIIIIILIIIVIIIIIIIIIIIIIIII |
| 402 | A | 0.947 | #AAAA-AAAAAAAAAAAAAAAAAAAAAAAAAAAAAAAAAAAAAAAAAAAAAAAAAAAAAAASAA |
| 403 | K | 0.728 | #KKRKKRKKNRKRRKRTKKRKKKRRKRKRKKRKKKRKKRRKKRRKRRRKKKKKRKRKRRKRKRR |

Table 1I

| | | | |
|---|---|---|---|
| 404 | N | 0.92 | #NNNNNNNNNNNNNNNNYNHNNNNNNNNNNNNNNNNNNINNNNNNNNNNNNNNNNNNNNNNNNN |
| 405 | C | 1 | #CCCCCCCCCCCCCCCCCCCCCCCCCCCCCCCCCCCCCCCCCCCCCCCCCCCCCCCCCCCCCCC |
| 406 | R | 0.937 | #RRRRRRRRRRRRKRRRRRRRRRRRRRRRRRRRRRRRRKRRRRRRRRRRRRRRRRRRKRRRRRR |
| 407 | A | 1 | #AAAAAAAAAAAAAAAAAAAAAAAAAAAAAAAAAAAAAAAAAAAAAAAAAAAAAAAAAAAAAAA |
| 408 | P | 1 | #PPPPPPPPPPPPPPPPPPPPPPPPPPPPPPPPPPPPPPPPPPPPPPPPPPPPPPPPPPPPPPP |
| 409 | R | 0.973 | #RRRRRRRRRRRRRRRRRRRRRRRRRRRRRRRKRRRRRRRRRRRRRRRRRRRRRRRRRRRRRRR |
| 410 | K | 0.927 | #KKKKKKKKKKRKKKKKIKKKKKKKRKKKKKKKKKKKKKKKKKKKKKKKKKKKKKKKKKKKKKK |
| 411 | K | 0.848 | #KKKKKKKKRKKRRKRRRRKKKKKKKKRKKKKKKKKKKKRKKKKKKKKKKKRKKKKKRKKKKKK |
| 412 | G | 0.973 | #GGGGGAGGGGGGGGGGGGGGGGGGGGGGGGGGGGGGGGGGGGGGGGGGGGGGGGGGGGGGGGG |
| 413 | C | 1 | #CCCCCCCCCCCCCCCCCCCCCCCCCCCCCCCCCCCCCCCCCCCCCCCCCCCCCCCCCCCCCCC |
| 414 | W | 0.911 | #WWWWC$WWWWWWWWWWWWWWWWWWWWWWWWW$WWWWWWWWW$WWWWWWWWWWWWWWWWWWWWW |
| 415 | K | 0.911 | #KRKRKKKKIKKKKKKKKKKKKKKKKKKKKKKKKKKKKKKKKKKKKKKKKRKKKKKKKKKKKKK |
| 416 | C | 0.973 | #CCCCCCCCCCCCCCCCCYCCCCCCCCCCCCCCCCCCCCCCCCCCCCCCCCCCCCCCCCCCCCC |
| 417 | G | 0.901 | #GGGGGEGGVGGGGGGGGGGGGGGGGGGGGGGRGGGGGGGGGRGGGGGGGGGGGGGGGGGGGGG |
| 418 | K | 0.883 | #KKKKKKKKKKKKKKKKKKRKKKKKKKKKRKKKKKKKKRKRKKKKKKKKKRKKKKKKKKKQKKK |
| 419 | E | 0.973 | #EEEEEEEEEEEEEEEEVEEEEEEEEEEEEEEEEEEEEEEEEEEEEEEEEEEEEEEEEEEEEEE |
| 420 | G | 0.973 | #GGGGGGGGGGGGGGGGGGGGGGGGGGGGGGGRGGGGGGGGGGGGGGGGGGGGGGGGGGGGGGG |
| 421 | H | 1 | #HHHHHHHHHHHHHHHHHHHHHHHHHHHHHHHHHHHHHHHHHHHHHHHHHHHHHHHHHHHHHHH |
| 422 | Q | 0.927 | #QQQQQQQQQQQQQQQHQQQLLQQQQQQQQQQQQQQQQQQQQQQQQQQQQQQQQQQQQQQQQQQ |
| 423 | M | 0.973 | #MMMMMMMMMMMMMMMMMMMMMMMMMMMMMMMMLMMMMMMMMMMMMMMMMMMMMMMMMMMMMMM |
| 424 | K | 0.947 | #KKKKKKKKKKKKKKKTIKKKKKKKKKKKKKKKKKKKKKKKKKKKKKKKKKKKKKKKKKKKKKK |
| 425 | D | 0.954 | #DDDDDDDDDDDDDDDDDDDDDDEDDDDDDDEDDDDDDDDDDDDDDDDDDDDDDDDDDDDDDDD |
| 426 | C | 1 | #CCCCCCCCCCCCCCCCCCCCCCCCCCCCCCCCCCCCCCCCCCCCCCCCCCCCCCCCCCCCCCC |
| 427 | T | 0.865 | #TTSTTTTTTTTTTTTTTTTTTTTTTTTTTNTTTTTTITTTITNTTTITTTTTTTTTTTTTTTT |
| 428 | E | 0.901 | #EEEEEKEEEEEEEEEDEEEEEEEEEEEEEEEEEEEEEEEEEKEEEEEENEEEEEEEEEEEEEE |
| 429 | R | 1 | #RRRRRRRRRRRRRRRRRRRRRRRRRRRRRRRRRRRRRRRRRRRRRRRRRRRRRRRRRRRRRRR |
| 430 | Q | 1 | #QQQQQQQQQQQQQQQQQQQQQQQQQQQQQQQQQQQQQQQQQQQQQQQQQQQQQQQQQQQQQQQ |
| 431 | A | 0.937 | #AVAVAVAAAAAAAAAAAAAAAAAAAAAAAAAAAAAAAAAAAAAAAAAAAAAAAAAAAAAAAAA |
| 432 | N | 1 | #NNNNNNNNNNNNNNNNNNNNNNNNNNNNNNNNNNNNNNNNNNNNNNNNNNNNNNNNNNNNNNN |
| 433 | F | 1 | #FFFFFFFFFFFFFFFFFFFFFFFFFFFFFFFFFFFFFFFFFFFFFFFFFFFFFFFFFFFFFFF |
| 434 | L | 1 | #LLLLLLLLLLLLLLLLLLLLLLLLLLLLLLLLLLLLLLLLLLLLLLLLLLLLLLLLLLLLLLL |
| 435 | G | 0.954 | #GGGGGGGGGGGGGGGGGGGGGGGGGGGGGGGRGGGGGGGGGRGGGGGGGGGGGGGGGGGGGGG |
| 436 | K | 0.973 | #KKRKKKKKKKKKKKKKKKKKKKKKKKKKKKKKKKKKKKKKKKKKKKKKKKKKKKKKKKKKKKK |
| 437 | I | 0.973 | #IIIIIIIIIIIIIIIIIIIIIILIIIIIIIIIIIIIIIIIIIIIIIIIIIIIIIIIIIIIIII |
| 438 | W | 1 | #WWWWWWWWWWWWWWWWWWWWWWWWWWWWWWWWWWWWWWWWWWWWWWWWWWWWWWWWWWWWWWW |
| 439 | P | 0.973 | #PPPPPPPPPPPPPPPPPPPPPPPPPPPPPPPPPPPPPPPPPPPPPPPPPPPPPPPPPPPSPPP |
| 440 | S | 1 | #SSSSSSSSSSSSSSSSSSSSSSSSSSSSSSSSSSSSSSSSSSSSSSSSSSSSSSSSSSSSSSS |
| 441 | H | 0.781 | #HHHHHHHHHHHHYHHYHHHHPHHHYHHYHHHHHHHHHHHYHHSHHHHHHYCHHHYHHHHQHHH |
| 442 | K | 0.947 | #KKKKKKKKKKKKKKKKKKKKKKKKKKRKKKKKSKKKKKKKKKKKKKKKKKKKKKKKKKKKKKK |
| 443 | G | 0.937 | #GGEGGGGGGGGGGGGGGGGGGGGGGGGGGGGGGGGGGGGGGGGGGEGGGGGGGGGGGGGGEGG |
| 444 | R | 1 | #RRRRRRRRRRRRRRRRRRRRRRRRRRRRRRRRRRRRRRRRRRRRRRRRRRRRRRRRRRRRRRR |
| 445 | P | 0.947 | #PPPPPPPPPPPPPPPPPQPPPPPPPPPPPPPPPPPPPPPPPPPPPPPPPP-PPPPPPPPPPPP |
| 446 | G | 0.881 | #GGGGGGGGGGGGGGGGGEGGGGGGGGGGGGGKGGGGGGGGERGGGGGGGGRGGGGGGGGGGGG |
| 447 | N | 1 | #NNNNNNNNNNNNNNNNNNNNNNNNNNNNNNNNNNNNNNNNNNNNNNNNNNNNNNNNNNNNNNN |
| 448 | F | 1 | #FFFFFFFFFFFFFFFFFFFFFFFFFFFFFFFFFFFFFFFFFFFFFFFFFFFFFFFFFFFFFFF |
| 449 | L | 0.885 | #LLPLPLLLLLLLLLLLLLLLLLLLLLPLLLLLLLPLLLLLLLLLLLLLLLPLLLLLPLLPLLL |
| 450 | Q | 1 | #QQQQQQQQQQQQQQQQQQQQQQQQQQQQQQQQQQQQQQQQQQQQQQQQQQQQQQQQQQQQQQQ |
| 451 | S | 0.852 | #SSRSSSSSSNSSSSSSSSSSSSSSRSNSNSSSSSSSSSSNSSSSSSSSSSSSSSSSSNSSSSN |
| 452 | R | 1 | #RRRRRRRRRRRRRRRRRRRRRRRRRRRRRRRRRRRRRRRRRRRRRRRRRRRRRRRRRRRRRRR |
| 453 | P | 0.809 | #PPLPAPLLTPPPPPPPPPPPPPPPPPLPPPPPPPTPPPPPPPPPPPPPPPTPPPPPLPPLPPP |
| 454 | E | 1 | #EEEEEEEEEEEEEEEEEEEEEEEEEEEEEEEEEEEEEEEEEEEEEEEEEEEEEEEEEEEEEEE |

Table 1J

| | | | |
|---|---|---|---|
| 455 | P | 1 | #PPPPPPPPPPPPPPPPPPPPPPPPPPPPPPPPPPPPPPPPPPPPPPPPPPPPPPPPPPPPPPP |
| 456 | T | 0.839 | #TTTTTSSSTSTTTTTTTSTTTTTTTTTTTTITTTTTSTTSTTTSTTTTTTTTTTTTTTTTSTT |
| 457 | A | 1 | #AAAAAAAAAAAAAAAAAAAAAAAAAAAAAAAAAAAAAAAAAAAAAAAAAAAAAAAAAAAAAAA |
| 458 | P | 0.973 | #PPPPPPPPPQPPPPPPPPPPPPPPPPPPPPPPPPPPPPPPPPPPPPPPPPPPPPPPPPPPPPP |
| 459 | P | 0.927 | #PPPPPPPPPSPPPPLPPPPPPPPPPPPPPPPPPPPPPPPPPPPPPPPPPPPPPPPSPPPPPPP |
| 460 | E | 0.805 | #EEEEEAAAAREEAEQEEEEEEEEEEEAEAEEEEEEEEEEAEEEEEEEEEEEEEEEEEAEEEEA |
| 461 | E | 0.937 | #EEEEEEEEEEEEEEEEEEEEEEEEEEEEEEEKEEEEEEEEEKEEEEEEEEEEEEKEEEEEEEE |
| 462 | S | 0.973 | #SSSSSSSSSSSSSSSSSSSSSSSSSSSSSSSSSSSSSSSISSSSSSSSSSSSSSSSSSSSSSS |
| 463 | F | 0.947 | #FFFFFFFFFFFFFFFFFFFFFFFFFFFFFFFFFFFFFFFFFFFFFFLFFFFFFFFVFFFFFFF |
| 464 | R | 0.896 | #RRRRRRGGRRRRRRRRRRRRRRRRRRRRGRRRRRRRRRRGRRRRRRRRRRRRRRRRRRRRRRK |
| 465 | F | 0.954 | #FFFFFFFFFFFFFFFFFFFFFFFFFFFSFFFFFFFFFFFFFFFFFFSFFFFFFFFFFFFFFFF |
| 466 | G | 0.855 | #GGGGGGGGGGGGGGGGGGGGGGGGGGGGGGGKGGGGGGRGEKGGGGGGGGGGGGGGGGGRGG- |
| 467 | E | 0.901 | #EEVEEEEEEEEEEEEEEEEEEEEEEEEVEEGEEEEEEEEEEEEEEEIEEEEEEEEEEEEEEEE |
| 468 | E | 0.855 | #EEEEEEGGEEEEEEEEEKEEEEEEE-EEEEEKEEEEEEEEEEEEEQEEEEEEEEEEEEEEEEE |
| 469 | T | 0.849 | #TTTTTTAAPTTTTTTTTTTTTTTTTATTTKTTKTTTTTTTTTTTTTTTTKTTTTTTTTTTTTT |
| 470 | T | 0.87 | #TTATTTTTTATTTTTTTTTTTTTTATTTTTTTTTTTATTTTTTTTTTT-TTTTTATTTTTATT |
| 471 | T | 0.861 | #TTTTTTAATTTTTTTTTTTTTTTTTATTTTTTTTATTTT-TTTTTTTTTTTTTTTTTTTTT-- |
| 472 | P | 0.973 | #PPPPPPPPPPPPPPPPPPPPPPPPPPPPPPPPPPSPPPPPPPPPPPPPPPPPPPPPPPPPPPP |
| 473 | S | 0.705 | #SPSPSPSSASASSSSSSPPSSSASFSSPPSSSSSPSSSSPPSSSSSSSSSYSSSSSPSSSSPT |
| 474 | Q | 0.954 | #QQQQQQQQQQQQQQQQQQQQQQQQQQQQQQQQQQQQPQQQQQQQQQQQQQQQQQQQQQQQQQP |
| 475 | K | 0.973 | #KKKKKKKKKKKKKKKKKKKKKKKKKKKKKKKKKKKKKKKKKKKKKKKKKKKKKKKKKKKKKKR |
| 476 | Q | 1 | #QQQQQQQQQQQQQQQQQQQQQQQQQQQQQQQQQQQQQQQQQQQQQQQQQQQQQQQQQQQQQQQ |
| 477 | E | 0.973 | #EEEEEEEEEEEEEEEEEEEEEEEEEEEEEEEEEEEEEEEEEREEEEEEEEEEEEEEEEEEEEE |
| 478 | P | 0.784 | #PPPPPPPPPPPPPPPSPPPPQPPPPPLPPPPPPPPPPPLPPTPPPPPPKPKPPPPPRPPPPPS |
| 479 | I | 0.765 | #IIIIIIIIRVIIIIRIIIIITIIIIIIIIIIIIILIIIIIIIRIRIIIIIIIIIIIEIII-IK |
| 480 | D | 0.92 | #DDDDDDDDDDDDDEDDDDDDDDDDDDDDDDNDDDDDDDDDDDDDDDDDDDDDDDDDDDDD-DD |
| 481 | K | 0.92 | #KKKKKKKKKKKKKKSKKKKKKKKKKKKKKKKKKKQKKKKKKKKKKKKKKKKKKKKKKKKKKKR |
| 482 | E | 0.8 | #EEEEEEEEDAEEEESEDEDEEEEEEEEEGEEDEEGEEEEEEEEEEDEEEEDEEEEEEEEEEEE |
| 483 | L | 0.71 | #L-LLLLLLLLLLLLLLLLLLLLLLLLLLLLPLLTKTLMMKLLLLLLVLLLLLQLMLMLLLLL- |
| 484 | Y | 0.92 | #YLYYYYYHYYYYYYYYYYYYYYYYYYYYYYYYYYYYYYYYYYYYYYYYYYYYYYYYYYYYYY- |
| 485 | P | 1 | #PPPPPPPPPPPPPPPPPPPPPPPPPPPPPPPPPPPPPPPPPPPPPPPPPPPPPPPPPPPPPPP |
| 486 | L | 0.839 | #LLLLFTTTLLLLLLLLLLLLLLLVLLSLLLLLLLLLLLSLLLLLLLLLLLLLLLLLLLLLLLL |
| 487 | A | 0.807 | #AAAASTAAAAAAAAATAAAAAATTAAATTAAAAAAAAAAAATAAAATAATAAATTAAAATAAT |
| 488 | S | 1 | #SSSSSSSSSSSSSSSSSSSSSSSSSSSSSSSSSSSSSSSSSSSSSSSSSSSSSSSSSSSSSSS |
| 489 | L | 1 | #LLLLLLLLLLLLLLLLLLLLLLLLLLLLLLLLLLLLLLLLLLLLLLLLLLLLLLLLLLLLLLL |
| 490 | R | 0.707 | #RKKKKRRSRKRRRKRRIRKKKKKKRKKRRRRKKRRRKRRRKRRRRERRKRKRRKRRRRRKKKK |
| 491 | S | 0.954 | #SSSSSSSSPSPSSSSSSSSSSSSSSSSSSSSSSSSSSSSSSSSSSSSSSSSSSSSSSSSSSSS |
| 492 | L | 1 | #LLLLLLLLLLLLLLLLLLLLLLLLLLLLLLLLLLLLLLLLLLLLLLLLLLLLLLLLLLLLLLL |
| 493 | F | 1 | #FFFFFFFFFFFFFFFFFFFFFFFFFFFFFFFFFFFFFFFFFFFFFFFFFFFFFFFFFFFFFFF |
| 494 | G | 1 | #GGGGGGGGGGGGGGGGGGGGGGGGGGGGGGGGGGGGGGGGGGGGGGGGGGGGGGGGGGGGGGG |
| 495 | N | 0.865 | #NNSNNNNNNNNNNNNNNNNNNNNNNSNNNNNNNNNNNSNNSSNNNNNSNNNSNNNSNNNNNNS |
| 496 | D | 1 | #DDDDDDDDDDDDDDDDDDDDDDDDDDDDDDDDDDDDDDDDDDDDDDDDDDDDDDDDDDDDDDD |
| 497 | P | 0.973 | #PPPPPPPPPPPPPPPPPPPPPPPPPPPPPPPPHPPPPPPPPPPPPPPPPPPPPPPPPPPPPPP |
| 498 | S | 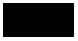 | #SSSSASSSSLSSSSSSSSSSLSSSSSLSSSSSLS$S$SSSSSSSSSSSSSLSSSSSSSSS$SS |
| 499 | S | 0.911 | #SSSSSSSSSSSSSSSSSSLSSSSSSSSSSSSLSSSSSSSSSSSSSSSSSSSSSSSSSSSS-LS |
| 500 | Q | 0.92 | #QQQQQQQQQQQQQQQQQQQQQQQQQQQQQQQLQQQQQQQQQQPQQQQQQQQQQQQQQQQQ-QQ |
| | | | #$$$$$$$$$$$$$$$$$$$$$$$$$$$$$$$$$$$$$$$$$$$$$$$$$$$$$$$$$$$$-$$ |

Sample #

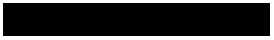

Pol

| | | | | | |
|---|---|---|---|---|---|
| | | | | Ranking | |
| AA# | Cons. | entropy | ratio:Cons. | of entropy | Raw data |

Table 2A

| | | | | | |
|---|---|---|---|---|---|
| 1 | F | 1 | 1 | 2310 | # FFFFFFFFFFFFFFFFFFFFFFFFFFFFFFFFFFFFFFFFF |
| 2 | F | 1 | 1 | 2311 | # FFFFFFFFFFFFFFFFFFFFFFFFFFFFFFFFFFFFFFFFF |
| 3 | R | 0.963 | 0.97560976 | 1361 | # RRRRKRRRRRRRRRRRRRRRRRRRRRRRRRRRRRRRRRRRR |
| 4 | E | 0.963 | 0.97560976 | 1362 | # EEEEEEEEEEEKEEEEEEEEEEEEEEEEEEEEEEEEEEEEE |
| 5 | D | 0.797 | 0.68292683 | 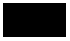 | # DNDNNDDDNDNNNDDDDDDNDDDNDNDDDDDDDDNNDNDDD |
| 6 | L | 0.963 | 0.97560976 | 1363 | # LMLLLLLLLLLLLLLLLLLLLLLLLLLLLLLLLLLLLLLLL |
| 7 | A | 0.963 | 0.97560976 | 1364 | # AAAAAAAAAAAAAAAAAAAAAAAAAAAAAAAAAAAAVAAAA |
| 8 | F | 0.937 | 0.95121951 | 1079 | # LFFFFLFFFFFFFFFFFFFFFFFFFFFFFFFFFFFFFFFFF |
| 9 | P | 0.785 | 0.73170732 | 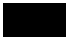 | # PQPPPPLPPLPPPPLPLLPPPPPPPPLLPPLLPPPPPPLPP |
| 10 | Q | 0.937 | 0.95121951 | 1080 | # QQQQQQQQQQQQQQQQQQQQQQQQQQQQQQQQQQQQKQKQQ |
| 11 | G | 0.897 | 0.90243902 | 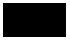 | # GRGRGGGGGGGGRGGGGGGGGGGGGGGGGGGGGGGGGGRGG |
| 12 | K | 0.829 | 0.78048786 | 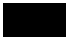 | # KKKKKKKKEKEEEKKEKKKEKKKKKKKKKEKKKKKKKKEKE |
| 13 | A | 1 | 1 | 2312 | # AAAAAAAAAAAAAAAAAAAAAAAAAAAAAAAAAAAAAAAAA |
| 14 | R | 0.926 | 0.95121951 | 952 | # RRRRRRGRRRRRRRRRRRRRRRRRRRR-RRRRRRRRRRRRR |
| 15 | E | 0.88 | 0.87804878 | 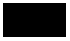 | # EEEEEEKEEKEEEEEKEEEEEEEKEEEEEEEEKEEEEEEEE |
| 16 | F | 0.865 | 0.85365854 | 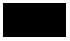 | # FFFFFLFFLLFLFLFFFFFFFFFFFLFFFFFFFFFFFFFFF |
| 17 | S | 0.88 | 0.87804878 | 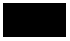 | # SPSSSSSSSSSSSSSSSSSSSPSSSSSSSSPSSSPSSSSSP |
| 18 | S | 0.879 | 0.90243902 | 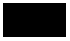 | # SSPSSSSSSSSSSSSSSSSSPSSSSSSSSTSSSSSSSSSSP |
| 19 | E | 0.926 | 0.95121951 | 953 | # EEKEEEEEEEEEEEAEEEEEEEEEEEEEEEEEEEEEEEEEE |
| 20 | Q | 1 | 1 | 2313 | # QQQQQQQQQQQQQQQQQQQQQQQQQQQQQQQQQQQQQQQQQ |
| 21 | T | 0.875 | 0.90243902 | 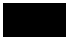 | # TTATTTTTATTTTTTTTTTTTTTTTTTNTTTTTTTTTTTTN |
| 22 | R | 0.901 | 0.92682927 | 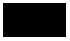 | # RGRRRRRRGRRRRRRRRRRRRRRRRRRRRRRRRRRRRRRRS |
| 23 | A | 0.926 | 0.95121951 | 954 | # AAAAAAAAAAAAAAAAAAAAAAAAAAAAAAAAAAAATAAAP |
| 24 | N | 0.825 | 0.85365854 | 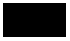 | # NNNNNNNNINNINNNNNNNNDINNNNNNNNNNNNNNNINNT |
| 25 | S | 1 | 1 | 2314 | # SSSSSSSSSSSSSSSSSSSSSSSSSSSSSSSSSSSSSSSSS |
| 26 | P | 0.963 | 0.97560976 | 1365 | # PPPPPPPPPPPPPPPPPPPPPPPPPPPPPPPPPSPPPPPPP |
| 27 | T | 0.926 | 0.95121951 | 955 | # TTTTTTTTTTTTTTTTTTTTTATTTTTTTTTTITTTTTTTT |
| 28 | R | 0.897 | 0.90243902 | 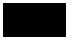 | # RSRRRRSRRRRRRRRRRRSRRRRRRRRRRRRRRRSRRRRRR |
| 29 | R | 0.861 | 0.87804878 | 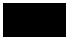 | # GRRRRRRRRRRRGGRRRRRRRRRGRRRRRRRKRRRRRRRRR |
| 30 | E | 1 | 1 | 2315 | # EEEEEEEEEEEEEEEEEEEEEEEEEEEEEEEEEEEEEEEEE |
| 31 | L | 0.926 | 0.95121951 | 956 | # LLLLLLLLLLLLLLLLLLLLLLLLLLLLLLLLRLLLLLPLL |
| 32 | Q | 0.963 | 0.97560976 | 1366 | # QQQQQQQQQQQQQQQQQQRQQQQQQQQQQQQQQQQQQQQQQ |
| 33 | V | 0.926 | 0.95121951 | 957 | # VLVVVVVVVVVVVVVVVVVVVVVVVVVVVVVVVVVVVV-VV |
| 34 | W | 0.864 | 0.90243902 | 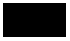 | # WWWW$WWWWWWWWWWWWWWWRWWWWWWWWWWWWWWWQW-WW |
| 35 | G | 0.963 | 0.97560976 | 1367 | # GGGGGGGGGGGGGGGGGGGGGGGGGGGGGGGGRGGGGGGGG |
| 36 | R | 0.679 | 0.6097561 | 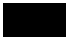 | # RGGGRRRGRGGERRRSRRRRRRRTRRGRRGRRRGRGRGSGR |
| 37 | D | 0.854 | 0.87804878 | 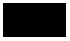 | # DDDNDDDDDDDNDDDNDDDEDDDDDDEDDDDDDDDDDDDDD |
| 38 | N | 0.785 | 0.7804878 | 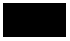 | # NNNSSNNNNNNNNNS-NNNNNNNNN-NNNNSSNNNNNSN-N |
| 39 | N | 0.926 | 0.95121951 | 958 | # NNNNNNNNNNNHNNNSNNNNNNNNNNNNNNNNNNNNNNNNN |
| 40 | S | 0.963 | 0.97560976 | 1368 | # SSSSSSSSSSSSPSSSSSSSSSSSSSSSSSSSSSSSSSSSS |
| 41 | L | 0.725 | 0.70731707 | 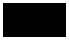 | # CRPPLLLLLLLPLILLPPPLLLLLLLLLLLLLLPLLLLLPP |
| 42 | S | 0.926 | 0.95121951 | 959 | # SSSSSSSSSPSSSSSSSSSSTSSSSSSSSSSSSSSSSSSSS |
| 43 | E | 1 | 1 | 2316 | # EEEEEEEEEEEEEEEEEEEEEEEEEEEEEEEEEEEEEEEEE |

Table 2B

| | | | | | |
|---|---|---|---|---|---|
| 44 | A | 0.963 | 0.97560976 | 1369 | # AAAAAAAAAAAAAAAAAAAATAAAAAAAAAAAAAAAAAAAA |
| 45 | G | 1 | 1 | 2317 | # GGGGGGGGGGGGGGGGGGGGGGGGGGGGGGGGGGGGGGGGG |
| 46 | A | 0.8 | 0.82926829 | 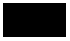 | # TAAATAAAAVAATAAAAAAAAAAAAEAEAAAAAAAAAATAA |
| 47 | D | 0.737 | 0.73170732 | 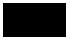 | # DDEEDDDDDDDDEDDDDDDDDNEDDDDEDDEDDGDDNEDND |
| 48 | R | 0.843 | 0.87804878 | 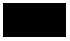 | # RRGGRRRRRRRRRRRRRRRRKRGRRRRRRRRRRRRRR-RRR |
| 49 | Q | 0.89 | 0.92682927 | 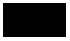 | # QSQQQQQEQQQQQQQQQQQQQQQQQQQQQQQQQQQQQ-QQQ |
| 50 | G | 1 | 1 | 2318 | # GGGGGGGGGGGGGGGGGGGGGGGGGGGGGGGGGGGGGGGGG |
| 51 | T | 0.705 | 0.75609756 | 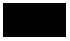 | # TETATTTTPTPTTTTTTTTTTTTPTTTPTTINTNTTATTTT |
| 52 | V | 0.915 | 0.92682927 | 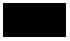 | # VVVIVVVVVVVVVIVVVVVVVVVVVVVVVVVVVVVVVIVVV |
| 53 | S | 1 | 1 | 2319 | # SSSSSSSSSSSSSSSSSSSSSSSSSSSSSSSSSSSSSSSSS |
| 54 | F | 0.811 | 0.73170732 | 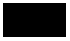 | # LFLLLFFFFLFFFFFLFFFFFFLFFFFFFFFFFLFLFLLFF |
| 55 | S | 0.727 | 0.68292683 | 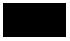 | # SNDNSSSSSNSSGSSSNNNSSSSSSSNSSDNNSSSSNSSSS |
| 56 | F | 0.844 | 0.85365854 | 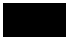 | # FCFLFFFFFFFFFFFLFFLFFFLFFFFFFFFFFFFFFLFFF |
| 57 | P | 1 | 1 | 2320 | # PPPPPPPPPPPPPPPPPPPPPPPPPPPPPPPPPPPPPPPPP |
| 58 | Q | 0.926 | 0.95121051 | 960 | # QQQQQQQQQQHQQQQQQQQQQQQRQQQQQQQQQQQQQQQQQ |
| 59 | I | 0.901 | 0.92682927 | 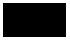 | # TIIIIIIIIIIIIIIIVVIIIIIIIIIIIIIIIIIIIIIII |
| 60 | T | 1 | 1 | 2321 | # TTTTTTTTTTTTTTTTTTTTTTTTTTTTTTTTTTTTTTTTT |
| 61 | L | 1 | 1 | 2322 | # LLLLLLLLLLLLLLLLLLLLLLLLLLLLLLLLLLLLLLLLL |
| 62 | W | 1 | 1 | 2323 | # WWWWWWWWWWWWWWWWWWWWWWWWWWWWWWWWWWWWWWWWW |
| 63 | Q | 1 | 1 | 2324 | # QQQQQQQQQQQQQQQQQQQQQQQQQQQQQQQQQQQQQQQQQ |
| 64 | R | 1 | 1 | 2325 | # RRRRRRRRRRRRRRRRRRRRRRRRRRRRRRRRRRRRRRRRR |
| 65 | P | 1 | 1 | 2326 | # PPPPPPPPPPPPPPPPPPPPPPPPPPPPPPPPPPPPPPPPP |
| 66 | L | 0.84 | 0.80487805 | 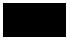 | # LLIILILLLLLLLLLLLLILILLLLILILILLLLLLLLLLL |
| 67 | V | 1 | 1 | 2327 | # VVVVVVVVVVVVVVVVVVVVVVVVVVVVVVVVVVVVVVVVV |
| 68 | T | 0.901 | 0.92682927 | 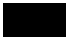 | # TTTTTTTTTTTTKTTTTTTTTATTTTTTTTTTTTTTTKTTT |
| 69 | I | 0.915 | 0.92682927 | 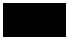 | # IIIIIIIVIIIIIIIIIIIIIIIIIVIIIIIIIIIIIIIIV |
| 70 | K | 0.937 | 0.95121951 | 1081 | # KKKKKKKKKKKKKKKKKKKKKKKRKKRKKKKKKKKKKKKKK |
| 71 | I | 0.865 | 0.85365854 | 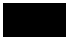 | # IVIIIIIVIIIIVVIIIIIIIIIIIIIIIIIIIIIVIIIVI |
| 72 | G | 0.926 | 0.95121951 | 961 | # GGGGKGGGGGGGGGGGGGGGGGGGGGGGGGGGGGGGEGGGG |
| 73 | G | 0.963 | 0.97560976 | 1370 | # GGGGRGGGGGGGGGGGGGGGGGGGGGGGGGGGGGGGGGGGG |
| 74 | Q | 1 | 1 | 2328 | # QQQQQQQQQQQQQQQQQQQQQQQQQQQQQQQQQQQQQQQQQ |
| 75 | L | 0.963 | 0.97560976 | 1371 | # LLLLLLLLLLLLLLLLLLLLLLLLLLLLLLLLLLILLLLLL |
| 76 | K | 0.963 | 0.97560976 | 1372 | # KRKKKKKKKKKKKKKKKKKKKKKKKKKKKKKKKKKKKKKKK |
| 77 | E | 0.963 | 0.97560976 | 1373 | # EEEEEEEEEEVEEEEEEEEEEEEEEEEEEEEEEEEEEEEEE |
| 78 | A | 1 | 1 | 2329 | # AAAAAAAAAAAAAAAAAAAAAAAAAAAAAAAAAAAAAAAAA |
| 79 | L | 1 | 1 | 2330 | # LLLLLLLLLLLLLLLLLLLLLLLLLLLLLLLLLLLLLLLLL |
| 80 | L | 1 | 1 | 2331 | # LLLLLLLLLLLLLLLLLLLLLLLLLLLLLLLLLLLLLLLLL |
| 81 | D | 1 | 1 | 2332 | # DDDDDDDDDDDDDDDDDDDDDDDDDDDDDDDDDDDDDDDDD |
| 82 | T | 1 | 1 | 2333 | # TTTTTTTTTTTTTTTTTTTTTTTTTTTTTTTTTTTTTTTTT |
| 83 | G | 1 | 1 | 2334 | # GGGGGGGGGGGGGGGGGGGGGGGGGGGGGGGGGGGGGGGGG |
| 84 | A | 1 | 1 | 2335 | # AAAAAAAAAAAAAAAAAAAAAAAAAAAAAAAAAAAAAAAAA |
| 85 | D | 1 | 1 | 2336 | # DDDDDDDDDDDDDDDDDDDDDDDDDDDDDDDDDDDDDDDDD |
| 86 | D | 1 | 1 | 2337 | # DDDDDDDDDDDDDDDDDDDDDDDDDDDDDDDDDDDDDDDDD |
| 87 | T | 1 | 1 | 2338 | # TTTTTTTTTTTTTTTTTTTTTTTTTTTTTTTTTTTTTTTTT |
| 88 | V | 1 | 1 | 2339 | # VVVVVVVVVVVVVVVVVVVVVVVVVVVVVVVVVVVVVVVVV |
| 89 | L | 0.937 | 0.95121951 | 1082 | # LLLLLLVLLLLLLLLVLLLLLLLLLLLLLLLLLLLLLLLLL |
| 90 | E | 0.963 | 0.97560976 | 1374 | # EEEEEEEEEEEEEEEEEEEEEEEEEEEGEEEEEEEEEEEEE |
| 91 | E | 0.769 | 0.68292683 | 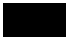 | # EEEEDEEEDEEKDDEEEEEEDEEEEEEEEDDDEEEDDEDED |

Table 2C

| | | | | | |
|---|---|---|---|---|---|
| 92 | M | 0.937 | 0.95121951 | 1083 | # MIMMMMMMMMMMMMMMMMMMMMMMMMMMMMMMMMMMMIMMM |
| 93 | N | 0.628 | 0.6097561 | 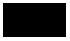 | # NDSNSCNNHNANNNSSSSNNNSNNNNNNNNDDNNNSNNDND |
| 94 | L | 1 | 1 | 2340 | # LLLLLLLLLLLLLLLLLLLLLLLLLLLLLLLLLLLLLLLLL |
| 95 | P | 1 | 1 | 2341 | # PPPPPPPPPPPPPPPPPPPPPPPPPPPPPPPPPPPPPPPPP |
| 96 | G | 0.937 | 0.95121951 | 1084 | # GGGGRGGGGGGGGGGGGGGGGGGGGGGRGGGGGGGGGGGGG |
| 97 | R | 0.84 | 0.80487805 | 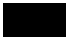 | # RRRRKRRRKRRRKRRRRRRRRRRKRKKRRRRRRRRRKKRRR |
| 98 | W | 0.963 | 0.97560976 | 1375 | # WWWW$WWWWWWWWWWWWWWWWWWWWWWWWWWWWWWWWWWWW |
| 99 | K | 1 | 1 | 2342 | # KKKKKKKKKKKKKKKKKKKKKKKKKKKKKKKKKKKKKKKKK |
| 100 | P | 1 | 1 | 2343 | # PPPPPPPPPPPPPPPPPPPPPPPPPPPPPPPPPPPPPPPPP |
| 101 | K | 0.963 | 0.97560976 | 1376 | # KKKKKKKKKKNKKKKKKKKKKKKKKKKKKKKKKKKKKKKKK |
| 102 | M | 1 | 1 | 2344 | # MMMMMMMMMMMMMMMMMMMMMMMMMMMMMMMMMMMMMMMMM |
| 103 | I | 1 | 1 | 2345 | # IIIIIIIIIIIIIIIIIIIIIIIIIIIIIIIIIIIIIIIII |
| 104 | G | 1 | 1 | 2346 | # GGGGGGGGGGGGGGGGGGGGGGGGGGGGGGGGGGGGGGGGG |
| 105 | G | 1 | 1 | 2347 | # GGGGGGGGGGGGGGGGGGGGGGGGGGGGGGGGGGGGGGGGG |
| 106 | I | 1 | 1 | 2348 | # IIIIIIIIIIIIIIIIIIIIIIIIIIIIIIIIIIIIIIIII |
| 107 | G | 0.963 | 0.97560976 | 1377 | # GGGGRGGGGGGGGGGGGGGGGGGGGGGGGGGGGGGGGGGGG |
| 108 | G | 0.963 | 0.97560976 | 1378 | # GGGGSGGGGGGGGGGGGGGGGGGGGGGGGGGGGGGGGGGGG |
| 109 | F | 1 | 1 | 2349 | # FFFFFFFFFFFFFFFFFFFFFFFFFFFFFFFFFFFFFFFFF |
| 110 | I | 1 | 1 | 2350 | # IIIIIIIIIIIIIIIIIIIIIIIIIIIIIIIIIIIIIIIII |
| 111 | K | 0.963 | 0.97560976 | 1379 | # KKKKKKKKKKRKKKKKKKKKKKKKKKKKKKKKKKKKKKKKK |
| 112 | V | 1 | 1 | 2351 | # VVVVVVVVVVVVVVVVVVVVVVVVVVVVVVVVVVVVVVVVV |
| 113 | R | 0.861 | 0.87804878 | 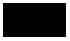 | # RRRRRRRRKRTRKRRRRRRRKRRRRRRRRRRRRRRRRRKRR |
| 114 | Q | 1 | 1 | 2352 | # QQQQQQQQQQQQQQQQQQQQQQQQQQQQQQQQQQQQQQQQQ |
| 115 | Y | 1 | 1 | 2353 | # YYYYYYYYYYYYYYYYYYYYYYYYYYYYYYYYYYYYYYYYY |
| 116 | D | 0.915 | 0.92682927 | 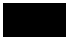 | # DDDDDDDDDDDDDDDDDDDDDDDDDDDDDDDDDDDEDDEDE |
| 117 | Q | 0.937 | 0.95121951 | 1085 | # QQQQQQQQEQQQQQQQQQQQQQQQQQQQQQQQQQQQQQQQE |
| 118 | I | 0.88 | 0.87804878 | 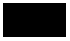 | # IIVIIVIIIIIIIIIIIIIIIVIIIIIIIIIIIIIIVVIII |
| 119 | L | 0.669 | 0.56097561 | 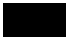 | # LTPLPLLLLLLLPPLLLLLPLAPPLLPTLLPLPPPDPLLLP |
| 120 | I | 0.829 | 0.7804878 | 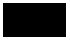 | # IVIVIVIIVIIVIIIVIIIIVIIIIIVIIIIIIIIIIIVII |
| 121 | E | 0.926 | 0.95121951 | 962 | # EEEEEEEEEEEEEEEEEEEEEEEEEEEGEEDEEEEEEEEEE |
| 122 | I | 1 | 1 | 2354 | # IIIIIIIIIIIIIIIIIIIIIIIIIIIIIIIIIIIIIIIII |
| 123 | C | 0.963 | 0.97560976 | 1380 | # CYCCCCCCCCCCCCCCCCCCCCCCCCCCCCCCCCCCCCCCC |
| 124 | G | 1 | 1 | 2355 | # GGGGGGGGGGGGGGGGGGGGGGGGGGGGGGGGGGGGGGGGG |
| 125 | H | 0.963 | 0.97560976 | 1381 | # HKHHHHHHHHHHHHHHHHHHHHHHHHHHHHHHHHHHHHHHH |
| 126 | K | 1 | 1 | 2356 | # KKKKKKKKKKKKKKKKKKKKKKKKKKKKKKKKKKKKKKKKK |
| 127 | A | 0.963 | 0.97560976 | 1382 | # AAAATAAAAAAAAAAAAAAAAAAAAAAAAAAAAAAAAAAAA |
| 128 | I | 0.926 | 0.95121951 | 963 | # IIIIIIIIIIIIIIIIIIIIIIIIIIIIIIVIIIIKIIIII |
| 129 | G | 1 | 1 | 2357 | # GGGGGGGGGGGGGGGGGGGGGGGGGGGGGGGGGGGGGGGGG |
| 130 | T | 1 | 1 | 2358 | # TTTTTTTTTTTTTTTTTTTTTTTTTTTTTTTTTTTTTTTTT |
| 131 | V | 1 | 1 | 2359 | # VVVVVVVVVVVVVVVVVVVVVVVVVVVVVVVVVVVVVVVVV |
| 132 | L | 1 | 1 | 2360 | # LLLLLLLLLLLLLLLLLLLLLLLLLLLLLLLLLLLLLLLLL |
| 133 | V | 0.937 | 0.95121951 | 1086 | # VVVVIVVVVVVVVVVVVVVVVIVVVVVVVVVVVVVVVVVVV |
| 134 | G | 1 | 1 | 2361 | # GGGGGGGGGGGGGGGGGGGGGGGGGGGGGGGGGGGGGGGGG |
| 135 | P | 1 | 1 | 2362 | # PPPPPPPPPPPPPPPPPPPPPPPPPPPPPPPPPPPPPPPPP |
| 136 | T | 1 | 1 | 2363 | # TTTTTTTTTTTTTTTTTTTTTTTTTTTTTTTTTTTTTTTTT |
| 137 | P | 1 | 1 | 2364 | # PPPPPPPPPPPPPPPPPPPPPPPPPPPPPPPPPPPPPPPPP |
| 138 | V | 1 | 1 | 2365 | # VVVVVVVVVVVVVVVVVVVVVVVVVVVVVVVVVVVVVVVVV |
| 139 | N | 1 | 1 | 2366 | # NNNNNNNNNNNNNNNNNNNNNNNNNNNNNNNNNNNNNNNNN |

Table 2D

| | | | | | |
|---|---|---|---|---|---|
| 140 | I | 1 | 1 | 2367 | # IIIIIIIIIIIIIIIIIIIIIIIIIIIIIIIIIIIIIIIII |
| 141 | I | 1 | 1 | 2368 | # IIIIIIIIIIIIIIIIIIIIIIIIIIIIIIIIIIIIIIIII |
| 142 | G | 0.963 | 0.97560976 | 1383 | # GGGGRGGGGGGGGGGGGGGGGGGGGGGGGGGGGGGGGGGGG |
| 143 | R | 0.963 | 0.97560976 | 1384 | # RRRRRRRRRRRKRRRRRRRRRRRRRRRRRRRRRRRRRRRRR |
| 144 | N | 0.963 | 0.97560976 | 1385 | # NNNNNNNNNDNNNNNNNNNNNNNNNNNNNNNNNNNNNNNNN |
| 145 | L | 1 | 1 | 2369 | # LLLLLLLLLLLLLLLLLLLLLLLLLLLLLLLLLLLLLLLLL |
| 146 | L | 1 | 1 | 2370 | # LLLLLLLLLLLLLLLLLLLLLLLLLLLLLLLLLLLLLLLLL |
| 147 | T | 1 | 1 | 2371 | # TTTTTTTTTTTTTTTTTTTTTTTTTTTTTTTTTTTTTTTTT |
| 148 | Q | 1 | 1 | 2372 | # QQQQQQQQQQQQQQQQQQQQQQQQQQQQQQQQQQQQQQQQQ |
| 149 | I | 0.829 | 0.7804878 | 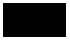 | # ILLILLIIIIIIILIIIILILIIIIIILILIIIIIIIIIII |
| 150 | G | 1 | 1 | 2373 | # GGGGGGGGGGGGGGGGGGGGGGGGGGGGGGGGGGGGGGGGG |
| 151 | C | 1 | 1 | 2374 | # CCCCCCCCCCCCCCCCCCCCCCCCCCCCCCCCCCCCCCCCC |
| 152 | T | 1 | 1 | 2375 | # TTTTTTTTTTTTTTTTTTTTTTTTTTTTTTTTTTTTTTTTT |
| 153 | L | 1 | 1 | 2376 | # LLLLLLLLLLLLLLLLLLLLLLLLLLLLLLLLLLLLLLLLL |
| 154 | N | 0.963 | 0.97560976 | 1386 | # NNNNNNNNNYNNNNNNNNNNNNNNNNNNNNNNNNNNNNNNN |
| 155 | F | 1 | 1 | 2377 | # FFFFFFFFFFFFFFFFFFFFFFFFFFFFFFFFFFFFFFFFF |
| 156 | P | 1 | 1 | 2378 | # PPPPPPPPPPPPPPPPPPPPPPPPPPPPPPPPPPPPPPPPP |
| 157 | I | 1 | 1 | 2379 | # IIIIIIIIIIIIIIIIIIIIIIIIIIIIIIIIIIIIIIIII |
| 158 | S | 0.963 | 0.97560976 | 1387 | # SSCSSSSSSSSSSSSSSSSSSSSSSSSSSSSSSSSSSSSSS |
| 159 | P | 1 | 1 | 2380 | # PPPPPPPPPPPPPPPPPPPPPPPPPPPPPPPPPPPPPPPPP |
| 160 | I | 1 | 1 | 2381 | # IIIIIIIIIIIIIIIIIIIIIIIIIIIIIIIIIIIIIIIII |
| 161 | E | 0.879 | 0.90243902 | 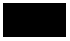 | # EEEEEEEEEDDEEKEEEEEEEEEDEEEEEEEEEEEEEEEEE |
| 162 | T | 1 | 1 | 2382 | # TTTTTTTTTTTTTTTTTTTTTTTTTTTTTTTTTTTTTTTTT |
| 163 | V | 1 | 1 | 2383 | # VVVVVVVVVVVVVVVVVVVVVVVVVVVVVVVVVVVVVVVVV |
| 164 | P | 1 | 1 | 2384 | # PPPPPPPPPPPPPPPPPPPPPPPPPPPPPPPPPPPPPPPPP |
| 165 | V | 1 | 1 | 2385 | # VVVVVVVVVVVVVVVVVVVVVVVVVVVVVVVVVVVVVVVVV |
| 166 | K | 0.963 | 0.97560976 | 1388 | # KKKKKKKKSKKKKKKKKKKKKKKKKKKKKKKKKKKKKKKKK |
| 167 | L | 1 | 1 | 2386 | # LLLLLLLLLLLLLLLLLLLLLLLLLLLLLLLLLLLLLLLLL |
| 168 | K | 1 | 1 | 2387 | # KKKKKKKKKKKKKKKKKKKKKKKKKKKKKKKKKKKKKKKKK |
| 169 | P | 1 | 1 | 2388 | # PPPPPPPPPPPPPPPPPPPPPPPPPPPPPPPPPPPPPPPPP |
| 170 | G | 0.926 | 0.95121951 | 964 | # GGGGRGGGGGGEGGGGGGGGGGGGGGGGGGGGGGGGGGGGG |
| 171 | M | 0.926 | 0.95121951 | 965 | # MMMMIMMMMMMMMMMMMMMMMMMMVMMMMMMMMMMMMMMMM |
| 172 | D | 0.963 | 0.97560976 | 1389 | # DDDDDDDDDDHDDDDDDDDDDDDDDDDDDDDDDDDDDDDDD |
| 173 | G | 0.963 | 0.97560976 | 1390 | # GGGGSGGGGGGGGGGGGGGGGGGGGGGGGGGGGGGGGGGGG |
| 174 | P | 1 | 1 | 2389 | # PPPPPPPPPPPPPPPPPPPPPPPPPPPPPPPPPPPPPPPPP |
| 175 | K | 0.963 | 0.97560976 | 1391 | # KKKKKKKKKKKKKKKKKKKKKKKKKKKKKKKKKKKKKRKKK |
| 176 | V | 0.963 | 0.97560976 | 1392 | # VIVVVVVVVVVVVVVVVVVVVVVVVVVVVVVVVVVVVVVVV |
| 177 | K | 0.963 | 0.97560976 | 1393 | # KKKKRKKKKKKKKKKKKKKKKKKKKKKKKKKKKKKKKKKKK |
| 178 | Q | 1 | 1 | 2390 | # QQQQQQQQQQQQQQQQQQQQQQQQQQQQQQQQQQQQQQQQQ |
| 179 | W | 1 | 1 | 2391 | # WWWWWWWWWWWWWWWWWWWWWWWWWWWWWWWWWWWWWWWWW |
| 180 | P | 1 | 1 | 2392 | # PPPPPPPPPPPPPPPPPPPPPPPPPPPPPPPPPPPPPPPPP |
| 181 | L | 1 | 1 | 2393 | # LLLLLLLLLLLLLLLLLLLLLLLLLLLLLLLLLLLLLLLLL |
| 182 | T | 0.963 | 0.97560976 | 1394 | # TTTTTTTTTTTTTTTTTTTTTTTTTTTTTTTTTTTTTSTTT |
| 183 | E | 1 | 1 | 2394 | # EEEEEEEEEEEEEEEEEEEEEEEEEEEEEEEEEEEEEEEEE |
| 184 | E | 1 | 1 | 2395 | # EEEEEEEEEEEEEEEEEEEEEEEEEEEEEEEEEEEEEEEEE |
| 185 | K | 1 | 1 | 2396 | # KKKKKKKKKKKKKKKKKKKKKKKKKKKKKKKKKKKKKKKKK |
| 186 | I | 1 | 1 | 2397 | # IIIIIIIIIIIIIIIIIIIIIIIIIIIIIIIIIIIIIIIII |
| 187 | K | 1 | 1 | 2398 | # KKKKKKKKKKKKKKKKKKKKKKKKKKKKKKKKKKKKKKKKK |

Table 2E

| | | | | | |
|---|---|---|---|---|---|
| 188 | A | 0.937 | 0.95121951 | 1087 | # AAAAAAAAAAAAAAAAAAVAAAAVAAAAAAAAAAAAAAAAA |
| 189 | L | 1 | 1 | 2399 | # LLLLLLLLLLLLLLLLLLLLLLLLLLLLLLLLLLLLLLLLL |
| 190 | V | 0.83 | 0.82926829 | 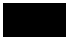 | # VVIVVVVVVVVVVVVIVVIVVVVIVVVIVVVVVVIVVTVVV |
| 191 | E | 1 | 1 | 2400 | # EEEEEEEEEEEEEEEEEEEEEEEEEEEEEEEEEEEEEEEEE |
| 192 | I | 1 | 1 | 2401 | # IIIIIIIIIIIIIIIIIIIIIIIIIIIIIIIIIIIIIIIII |
| 193 | C | 1 | 1 | 2402 | # CCCCCCCCCCCCCCCCCCCCCCCCCCCCCCCCCCCCCCCCC |
| 194 | T | 0.963 | 0.97560976 | 1395 | # TTTTTTTMTTTTTTTTTTTTTTTTTTTTTTTTTTTTTTTTT |
| 195 | E | 1 | 1 | 2403 | # EEEEEEEEEEEEEEEEEEEEEEEEEEEEEEEEEEEEEEEEE |
| 196 | M | 0.963 | 0.97560976 | 1396 | # MMMMMMMMMMMMMMMMMMMMMMMMMMMMMMMMMMMMMMLMM |
| 197 | E | 0.963 | 0.97560976 | 1397 | # EEEEEEEEEEEKEEEEEEEEEEEEEEEEEEEEEEEEEEEEE |
| 198 | K | 1 | 1 | 2404 | # KKKKKKKKKKKKKKKKKKKKKKKKKKKKKKKKKKKKKKKKK |
| 199 | E | 1 | 1 | 2405 | # EEEEEEEEEEEEEEEEEEEEEEEEEEEEEEEEEEEEEEEEE |
| 200 | G | 0.963 | 0.97560976 | 1398 | # GGGGRGGGGGGGGGGGGGGGGGGGGGGGGGGGGGGGGGGGG |
| 201 | K | 0.963 | 0.97560976 | 1399 | # KKRKKKKKKKKKKKKKKKKKKKKKKKKKKKKKKKKKKKKKK |
| 202 | I | 1 | 1 | 2406 | # IIIIIIIIIIIIIIIIIIIIIIIIIIIIIIIIIIIIIIIII |
| 203 | S | 1 | 1 | 2407 | # SSSSSSSSSSSSSSSSSSSSSSSSSSSSSSSSSSSSSSSSS |
| 204 | K | 0.937 | 0.95121951 | 1088 | # KKKKKKKKKKKRKKKKKKKKKKKKKKKKKRKKKKKKKKKKK |
| 205 | I | 0.963 | 0.97560976 | 1400 | # IIIIIIIIIIIIIIIIIIVIIIIIIIIIIIIIIIIIIIIII |
| 206 | G | 0.963 | 0.97560976 | 1401 | # GGGGRGGGGGGGGGGGGGGGGGGGGGGGGGGGGGGGGGGGG |
| 207 | P | 1 | 1 | 2408 | # PPPPPPPPPPPPPPPPPPPPPPPPPPPPPPPPPPPPPPPPP |
| 208 | E | 0.963 | 0.97560976 | 1402 | # EEEEEEEEEEEKEEEEEEEEEEEEEEEEEEEEEEEEEEEEE |
| 209 | N | 1 | 1 | 2409 | # NNNNNNNNNNNNNNNNNNNNNNNNNNNNNNNNNNNNNNNNN |
| 210 | P | 1 | 1 | 2410 | # PPPPPPPPPPPPPPPPPPPPPPPPPPPPPPPPPPPPPPPPP |
| 211 | Y | 1 | 1 | 2411 | # YYYYYYYYYYYYYYYYYYYYYYYYYYYYYYYYYYYYYYYYY |
| 212 | N | 1 | 1 | 2412 | # NNNNNNNNNNNNNNNNNNNNNNNNNNNNNNNNNNNNNNNNN |
| 213 | T | 1 | 1 | 2413 | # TTTTTTTTTTTTTTTTTTTTTTTTTTTTTTTTTTTTTTTTT |
| 214 | P | 1 | 1 | 2414 | # PPPPPPPPPPPPPPPPPPPPPPPPPPPPPPPPPPPPPPPPP |
| 215 | V | 0.963 | 0.97560976 | 1403 | # VVVVVVVVVVVVVVVVVVVVVVVVVVVVVVVVVVVVVIVVV |
| 216 | F | 1 | 1 | 2415 | # FFFFFFFFFFFFFFFFFFFFFFFFFFFFFFFFFFFFFFFFF |
| 217 | A | 1 | 1 | 2416 | # AAAAAAAAAAAAAAAAAAAAAAAAAAAAAAAAAAAAAAAAA |
| 218 | I | 1 | 1 | 2417 | # IIIIIIIIIIIIIIIIIIIIIIIIIIIIIIIIIIIIIIIII |
| 219 | K | 0.963 | 0.97560976 | 1404 | # KKKKKKKKRKKKKKKKKKKKKKKKKKKKKKKKKKKKKKKKK |
| 220 | K | 1 | 1 | 2418 | # KKKKKKKKKKKKKKKKKKKKKKKKKKKKKKKKKKKKKKKKK |
| 221 | K | 1 | 1 | 2419 | # KKKKKKKKKKKKKKKKKKKKKKKKKKKKKKKKKKKKKKKKK |
| 222 | D | 0.963 | 0.97560976 | 1405 | # DDDDDDDDDDDDDDDDDDDDDDDDDDDDDDDDDDDDDNDDD |
| 223 | S | 0.937 | 0.95121951 | 1089 | # SSSSSSSGGSSSSSSSSSSSSSSSSSSSSSSSSSSSSSSSS |
| 224 | T | 0.963 | 0.97560976 | 1406 | # TTTTTTTTTTTTTTTTTTTTTTTTTTTTTTTTTTTTTTTTN |
| 225 | K | 0.915 | 0.92682927 | 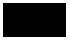 | # KKRKKKKKKKKKKKKKKKKKKKKKKKKKKRKKKKKKKRKKK |
| 226 | W | 0.963 | 0.97560976 | 1407 | # WWWW$WWWWWWWWWWWWWWWWWWWWWWWWWWWWWWWWWWWW |
| 227 | R | 1 | 1 | 2420 | # RRRRRRRRRRRRRRRRRRRRRRRRRRRRRRRRRRRRRRRRR |
| 228 | K | 1 | 1 | 2421 | # KKKKKKKKKKKKKKKKKKKKKKKKKKKKKKKKKKKKKKKKK |
| 229 | L | 1 | 1 | 2422 | # LLLLLLLLLLLLLLLLLLLLLLLLLLLLLLLLLLLLLLLLL |
| 230 | V | 1 | 1 | 2423 | # VVVVVVVVVVVVVVVVVVVVVVVVVVVVVVVVVVVVVVVVV |
| 231 | D | 1 | 1 | 2424 | # DDDDDDDDDDDDDDDDDDDDDDDDDDDDDDDDDDDDDDDDD |
| 232 | F | 1 | 1 | 2425 | # FFFFFFFFFFFFFFFFFFFFFFFFFFFFFFFFFFFFFFFFF |
| 233 | R | 1 | 1 | 2426 | # RRRRRRRRRRRRRRRRRRRRRRRRRRRRRRRRRRRRRRRRR |
| 234 | E | 1 | 1 | 2427 | # EEEEEEEEEEEEEEEEEEEEEEEEEEEEEEEEEEEEEEEEE |
| 235 | L | 1 | 1 | 2428 | # LLLLLLLLLLLLLLLLLLLLLLLLLLLLLLLLLLLLLLLLL |

Table 2F

| | | | | | |
|---|---|---|---|---|---|
| 236 | N | 1 | 1 | 2429 | # NNNNNNNNNNNNNNNNNNNNNNNNNNNNNNNNNNNNNNNNN |
| 237 | K | 0.963 | 0.97560976 | 1408 | # KKKKKKKKKKKKKKKKKKKKKKKKKKKKKKRKKKKKKKKKK |
| 238 | R | 0.88 | 0.87804878 | 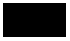 | # RRKRRRRRRKKRRRRRRRRRRRRRRRRKRRRKRRRRRRRRR |
| 239 | T | 1 | 1 | 2430 | # TTTTTTTTTTTTTTTTTTTTTTTTTTTTTTTTTTTTTTTTT |
| 240 | Q | 1 | 1 | 2431 | # QQQQQQQQQQQQQQQQQQQQQQQQQQQQQQQQQQQQQQQQQ |
| 241 | D | 1 | 1 | 2432 | # DDDDDDDDDDDDDDDDDDDDDDDDDDDDDDDDDDDDDDDDD |
| 242 | F | 1 | 1 | 2433 | # FFFFFFFFFFFFFFFFFFFFFFFFFFFFFFFFFFFFFFFFF |
| 243 | W | 0.963 | 0.97560976 | 1409 | # WWWW$WWWWWWWWWWWWWWWWWWWWWWWWWWWWWWWWWWWW |
| 244 | E | 1 | 1 | 2434 | # EEEEEEEEEEEEEEEEEEEEEEEEEEEEEEEEEEEEEEEEE |
| 245 | V | 0.963 | 0.97560976 | 1410 | # VVVVVVVVVVVVVAVVVVVVVVVVVVVVVVVVVVVVVVVVV |
| 246 | Q | 1 | 1 | 2435 | # QQQQQQQQQQQQQQQQQQQQQQQQQQQQQQQQQQQQQQQQQ |
| 247 | L | 1 | 1 | 2436 | # LLLLLLLLLLLLLLLLLLLLLLLLLLLLLLLLLLLLLLLLL |
| 248 | G | 1 | 1 | 2437 | # GGGGGGGGGGGGGGGGGGGGGGGGGGGGGGGGGGGGGGGGG |
| 249 | I | 1 | 1 | 2438 | # IIIIIIIIIIIIIIIIIIIIIIIIIIIIIIIIIIIIIIIII |
| 250 | P | 1 | 1 | 2439 | # PPPPPPPPPPPPPPPPPPPPPPPPPPPPPPPPPPPPPPPPP |
| 251 | H | 1 | 1 | 2440 | # HHHHHHHHHHHHHHHHHHHHHHHHHHHHHHHHHHHHHHHHH |
| 252 | P | 1 | 1 | 2441 | # PPPPPPPPPPPPPPPPPPPPPPPPPPPPPPPPPPPPPPPPP |
| 253 | A | 0.897 | 0.90243902 | 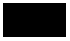 | # AAAAAAAASAASAAAAAAAASAAAAAAAAAAAAAAASAAAA |
| 254 | G | 1 | 1 | 2442 | # GGGGGGGGGGGGGGGGGGGGGGGGGGGGGGGGGGGGGGGGG |
| 255 | L | 1 | 1 | 2443 | # LLLLLLLLLLLLLLLLLLLLLLLLLLLLLLLLLLLLLLLLL |
| 256 | K | 0.963 | 0.97560976 | 1411 | # KKKKKKKKKKKKRKKKKKKKKKKKKKKKKKKKKKKKKKKKK |
| 257 | K | 0.937 | 0.95121951 | 1090 | # KKKKKKKKKKKKKKKKKKKKKKKKQKKKQKKKKKKKKKKKK |
| 258 | K | 0.963 | 0.97560976 | 1412 | # KKKKKKKKKKKKKKKKKKKKKKKKKKKKKKKRKKKKKKKKK |
| 259 | K | 0.963 | 0.97560976 | 1413 | # KKKKKKKKRKKKKKKKKKKKKKKKKKKKKKKKKKKKKKKKK |
| 260 | S | 1 | 1 | 2444 | # SSSSSSSSSSSSSSSSSSSSSSSSSSSSSSSSSSSSSSSSS |
| 261 | V | 0.926 | 0.95121951 | 966 | # VVVIVVVVVVVVVVVVVVVVVVGVVVVVVVVVVVVVVVVVV |
| 262 | T | 1 | 1 | 2445 | # TTTTTTTTTTTTTTTTTTTTTTTTTTTTTTTTTTTTTTTTT |
| 263 | V | 0.963 | 0.97560976 | 1414 | # VVVVVVVVVVVVVVVVVVVVVVAVVVVVVVVVVVVVVVVVV |
| 264 | L | 1 | 1 | 2446 | # LLLLLLLLLLLLLLLLLLLLLLLLLLLLLLLLLLLLLLLLL |
| 265 | D | 1 | 1 | 2447 | # DDDDDDDDDDDDDDDDDDDDDDDDDDDDDDDDDDDDDDDDD |
| 266 | V | 1 | 1 | 2448 | # VVVVVVVVVVVVVVVVVVVVVVVVVVVVVVVVVVVVVVVVV |
| 267 | G | 1 | 1 | 2449 | # GGGGGGGGGGGGGGGGGGGGGGGGGGGGGGGGGGGGGGGGG |
| 268 | D | 1 | 1 | 2450 | # DDDDDDDDDDDDDDDDDDDDDDDDDDDDDDDDDDDDDDDDD |
| 269 | A | 0.963 | 0.97560976 | 1415 | # AAAAAAAAAAAAAAAAAAAAAAAAAAAAAAAAARAAAAAAA |
| 270 | Y | 1 | 1 | 2451 | # YYYYYYYYYYYYYYYYYYYYYYYYYYYYYYYYYYYYYYYYY |
| 271 | F | 1 | 1 | 2452 | # FFFFFFFFFFFFFFFFFFFFFFFFFFFFFFFFFFFFFFFFF |
| 272 | S | 1 | 1 | 2453 | # SSSSSSSSSSSSSSSSSSSSSSSSSSSSSSSSSSSSSSSSS |
| 273 | V | 0.937 | 0.95121951 | 1091 | # VVVVIVVVVVVVVVVVVVVVVVVVVVVVVVVVVVVVVIVVV |
| 274 | P | 1 | 1 | 2454 | # PPPPPPPPPPPPPPPPPPPPPPPPPPPPPPPPPPPPPPPPP |
| 275 | L | 1 | 1 | 2455 | # LLLLLLLLLLLLLLLLLLLLLLLLLLLLLLLLLLLLLLLLL |
| 276 | D | 0.875 | 0.90243902 | 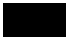 | # YDDDDDDYDDDDDDDDDDDDDDDDDDDDDDDDHDDDDDDDH |
| 277 | K | 0.782 | 0.6097561 | 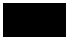 | # EKKKKKKEEEKEEKEKEEKKKEKKKKKKKKKKEKKEEEKEE |
| 278 | D | 0.784 | 0.80487805 | 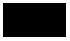 | # DDDDDDEDNNDSDDDDDDDDEDDDDEDDDDDDDDEDDDEDD |
| 279 | F | 1 | 1 | 2456 | # FFFFFFFFFFFFFFFFFFFFFFFFFFFFFFFFFFFFFFFFF |
| 280 | R | 1 | 1 | 2457 | # RRRRRRRRRRRRRRRRRRRRRRRRRRRRRRRRRRRRRRRRR |
| 281 | K | 1 | 1 | 2458 | # KKKKKKKKKKKKKKKKKKKKKKKKKKKKKKKKKKKKKKKKK |
| 282 | Y | 1 | 1 | 2459 | # YYYYYYYYYYYYYYYYYYYYYYYYYYYYYYYYYYYYYYYYY |
| 283 | T | 1 | 1 | 2460 | # TTTTTTTTTTTTTTTTTTTTTTTTTTTTTTTTTTTTTTTTT |

Table 2G

| | | | | | |
|---|---|---|---|---|---|
| 284 | A | 0.963 | 0.97560976 | 1416 | # VAAAAAAAAAAAAAAAAAAAAAAAAAAAAAAAAAAAAAAAA |
| 285 | F | 1 | 1 | 2461 | # FFFFFFFFFFFFFFFFFFFFFFFFFFFFFFFFFFFFFFFFF |
| 286 | T | 1 | 1 | 2462 | # TTTTTTTTTTTTTTTTTTTTTTTTTTTTTTTTTTTTTTTTT |
| 287 | I | 1 | 1 | 2463 | # IIIIIIIIIIIIIIIIIIIIIIIIIIIIIIIIIIIIIIIII |
| 288 | P | 1 | 1 | 2464 | # PPPPPPPPPPPPPPPPPPPPPPPPPPPPPPPPPPPPPPPPP |
| 289 | S | 1 | 1 | 2465 | # SSSSSSSSSSSSSSSSSSSSSSSSSSSSSSSSSSSSSSSSS |
| 290 | I | 0.767 | 0.75609756 | 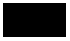 | # IIIITIITIIIITVIIIIITITVIIIIIIIIIIITIIVIIV |
| 291 | N | 1 | 1 | 2466 | # NNNNNNNNNNNNNNNNNNNNNNNNNNNNNNNNNNNNNNNNN |
| 292 | N | 1 | 1 | 2467 | # NNNNNNNNNNNNNNNNNNNNNNNNNNNNNNNNNNNNNNNNN |
| 293 | E | 0.963 | 0.97560976 | 1417 | # EEEEEEEEEEEEEEEEEEEEEEEEEEEEEEEEEEEEEAEEE |
| 294 | T | 0.937 | 0.95121951 | 1092 | # ATTTTTTTTTTTTTTTTTTTTTTTTTTTTTTTTTTTTATTT |
| 295 | P | 1 | 1 | 2468 | # PPPPPPPPPPPPPPPPPPPPPPPPPPPPPPPPPPPPPPPPP |
| 296 | G | 0.926 | 0.95121951 | 967 | # GGGGEGGGGGGGGGGGGGGGGGGGGRGGGGGGGGGGGGGGG |
| 297 | I | 0.875 | 0.90243902 | 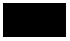 | # VIIIIIIIIIITIIVIIIIIIIIIIIIIIIIITIIIIIIII |
| 298 | R | 1 | 1 | 2469 | # RRRRRRRRRRRRRRRRRRRRRRRRRRRRRRRRRRRRRRRRR |
| 299 | Y | 1 | 1 | 2470 | # YYYYYYYYYYYYYYYYYYYYYYYYYYYYYYYYYYYYYYYYY |
| 300 | Q | 1 | 1 | 2471 | # QQQQQQQQQQQQQQQQQQQQQQQQQQQQQQQQQQQQQQQQQ |
| 301 | Y | 1 | 1 | 2472 | # YYYYYYYYYYYYYYYYYYYYYYYYYYYYYYYYYYYYYYYYY |
| 302 | N | 1 | 1 | 2473 | # NNNNNNNNNNNNNNNNNNNNNNNNNNNNNNNNNNNNNNNNN |
| 303 | V | 1 | 1 | 2474 | # VVVVVVVVVVVVVVVVVVVVVVVVVVVVVVVVVVVVVVVVV |
| 304 | L | 1 | 1 | 2475 | # LLLLLLLLLLLLLLLLLLLLLLLLLLLLLLLLLLLLLLLLL |
| 305 | P | 1 | 1 | 2476 | # PPPPPPPPPPPPPPPPPPPPPPPPPPPPPPPPPPPPPPPPP |
| 306 | Q | 1 | 1 | 2477 | # QQQQQQQQQQQQQQQQQQQQQQQQQQQQQQQQQQQQQQQQQ |
| 307 | G | 0.963 | 0.97560976 | 1418 | # GGGGRGGGGGGGGGGGGGGGGGGGGGGGGGGGGGGGGGGGG |
| 308 | W | 1 | 1 | 2478 | # WWWWWWWWWWWWWWWWWWWWWWWWWWWWWWWWWWWWWWWWW |
| 309 | K | 1 | 1 | 2479 | # KKKKKKKKKKKKKKKKKKKKKKKKKKKKKKKKKKKKKKKKK |
| 310 | G | 1 | 1 | 2480 | # GGGGGGGGGGGGGGGGGGGGGGGGGGGGGGGGGGGGGGGGG |
| 311 | S | 1 | 1 | 2481 | # SSSSSSSSSSSSSSSSSSSSSSSSSSSSSSSSSSSSSSSSS |
| 312 | P | 1 | 1 | 2482 | # PPPPPPPPPPPPPPPPPPPPPPPPPPPPPPPPPPPPPPPPP |
| 313 | A | 1 | 1 | 2483 | # AAAAAAAAAAAAAAAAAAAAAAAAAAAAAAAAAAAAAAAAA |
| 314 | I | 1 | 1 | 2484 | # IIIIIIIIIIIIIIIIIIIIIIIIIIIIIIIIIIIIIIIII |
| 315 | F | 1 | 1 | 2485 | # FFFFFFFFFFFFFFFFFFFFFFFFFFFFFFFFFFFFFFFFF |
| 316 | Q | 1 | 1 | 2486 | # QQQQQQQQQQQQQQQQQQQQQQQQQQQQQQQQQQQQQQQQQ |
| 317 | S | 0.805 | 0.7804878 | 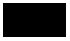 | # SSSSSSSCSSYSCCSSSSSSSSCSCSSSCSSSSSSSSSSCC |
| 318 | S | 1 | 1 | 2487 | # SSSSSSSSSSSSSSSSSSSSSSSSSSSSSSSSSSSSSSSSS |
| 319 | M | 1 | 1 | 2488 | # MMMMMMMMMMMMMMMMMMMMMMMMMMMMMMMMMMMMMMMMM |
| 320 | T | 1 | 1 | 2489 | # TTTTTTTTTTTTTTTTTTTTTTTTTTTTTTTTTTTTTTTTT |
| 321 | K | 0.861 | 0.87804878 | 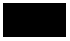 | # KKRKKKKKRKKKKKKKKKKKKKKRKKKKKKKKTKKKKKRKK |
| 322 | I | 1 | 1 | 2490 | # IIIIIIIIIIIIIIIIIIIIIIIIIIIIIIIIIIIIIIIII |
| 323 | L | 1 | 1 | 2491 | # LLLLLLLLLLLLLLLLLLLLLLLLLLLLLLLLLLLLLLLLL |
| 324 | E | 1 | 1 | 2492 | # EEEEEEEEEEEEEEEEEEEEEEEEEEEEEEEEEEEEEEEEE |
| 325 | P | 1 | 1 | 2493 | # PPPPPPPPPPPPPPPPPPPPPPPPPPPPPPPPPPPPPPPPP |
| 326 | F | 1 | 1 | 2494 | # FFFFFFFFFFFFFFFFFFFFFFFFFFFFFFFFFFFFFFFFF |
| 327 | R | 0.963 | 0.97560976 | 1419 | # RRRRRRRRRRRRRRRRRRRRRRRRRKRRRRRRRRRRRRRRR |
| 328 | K | 0.926 | 0.95121951 | 968 | # KKKKKKKKRKKKKKKKKKKKKKKKKKKKKKKKKKKKKKKQK |
| 329 | Q | 0.937 | 0.95121951 | 1093 | # QHQQQQQQQQQQQQQQQQQQQQQQQQQQQQQQQQQQQQQQH |
| 330 | N | 1 | 1 | 2495 | # NNNNNNNNNNNNNNNNNNNNNNNNNNNNNNNNNNNNNNNNN |
| 331 | P | 1 | 1 | 2496 | # PPPPPPPPPPPPPPPPPPPPPPPPPPPPPPPPPPPPPPPPP |

Table 2H

| | | | | | |
|---|---|---|---|---|---|
| 332 | D | 0.865 | 0.85365854 | 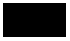 | # DDDDDDEEDDDDDDDDDDDDEDDDDEDDDDDDDDEDDDDDE |
| 333 | I | 0.864 | 0.90243902 | 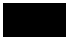 | # VMIIIVIIIIIIIIIIIIIIIIIIIIIIIIIILIIIIIIII |
| 334 | V | 0.937 | 0.95121951 | 1094 | # VVVVVVVVVVVVVVVVVVVVVVVVVVVVVVIIVVVVVVVVV |
| 335 | I | 1 | 1 | 2497 | # IIIIIIIIIIIIIIIIIIIIIIIIIIIIIIIIIIIIIIIII |
| 336 | Y | 1 | 1 | 2498 | # YYYYYYYYYYYYYYYYYYYYYYYYYYYYYYYYYYYYYYYYY |
| 337 | Q | 1 | 1 | 2499 | # QQQQQQQQQQQQQQQQQQQQQQQQQQQQQQQQQQQQQQQQQ |
| 338 | Y | 1 | 1 | 2500 | # YYYYYYYYYYYYYYYYYYYYYYYYYYYYYYYYYYYYYYYYY |
| 339 | M | 0.963 | 0.97560976 | 1420 | # MMMMIMMMMMMMMMMMMMMMMMMMMMMMMMMMMMMMMMMMM |
| 340 | D | 1 | 1 | 2501 | # DDDDDDDDDDDDDDDDDDDDDDDDDDDDDDDDDDDDDDDDD |
| 341 | D | 1 | 1 | 2502 | # DDDDDDDDDDDDDDDDDDDDDDDDDDDDDDDDDDDDDDDDD |
| 342 | L | 1 | 1 | 2503 | # LLLLLLLLLLLLLLLLLLLLLLLLLLLLLLLLLLLLLLLLL |
| 343 | Y | 1 | 1 | 2504 | # YYYYYYYYYYYYYYYYYYYYYYYYYYYYYYYYYYYYYYYYY |
| 344 | V | 1 | 1 | 2505 | # VVVVVVVVVVVVVVVVVVVVVVVVVVVVVVVVVVVVVVVVV |
| 345 | G | 1 | 1 | 2506 | # GGGGGGGGGGGGGGGGGGGGGGGGGGGGGGGGGGGGGGGGG |
| 346 | S | 1 | 1 | 2507 | # SSSSSSSSSSSSSSSSSSSSSSSSSSSSSSSSSSSSSSSSS |
| 347 | D | 1 | 1 | 2508 | # DDDDDDDDDDDDDDDDDDDDDDDDDDDDDDDDDDDDDDDDD |
| 348 | L | 1 | 1 | 2509 | # LLLLLLLLLLLLLLLLLLLLLLLLLLLLLLLLLLLLLLLLL |
| 349 | E | 1 | 1 | 2510 | # EEEEEEEEEEEEEEEEEEEEEEEEEEEEEEEEEEEEEEEEE |
| 350 | I | 0.963 | 0.97560976 | 1421 | # IIIIIIIIIIIIIIIIIIIIIIIIIIIIIIIIIIIIIILII |
| 351 | G | 0.915 | 0.92682927 | 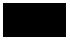 | # GGGGGGGGGGGGGGGEGGGGGGGGGGGGGGGGGGGGGEGGE |
| 352 | Q | 1 | 1 | 2511 | # QQQQQQQQQQQQQQQQQQQQQQQQQQQQQQQQQQQQQQQQQ |
| 353 | H | 1 | 1 | 2512 | # HHHHHHHHHHHHHHHHHHHHHHHHHHHHHHHHHHHHHHHHH |
| 354 | R | 1 | 1 | 2513 | # RRRRRRRRRRRRRRRRRRRRRRRRRRRRRRRRRRRRRRRRR |
| 355 | T | 0.781 | 0.7804878 | 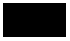 | # TTTTTTTTITTTTATTTTTTTITITITATTTATTTATTTTA |
| 356 | K | 1 | 1 | 2514 | # KKKKKKKKKKKKKKKKKKKKKKKKKKKKKKKKKKKKKKKKK |
| 357 | I | 0.937 | 0.95121951 | 1095 | # IIIIIIIIIIIIIIIIIIIIIIIVIIIIIIIIIIIIIIVII |
| 358 | E | 1 | 1 | 2515 | # EEEEEEEEEEEEEEEEEEEEEEEEEEEEEEEEEEEEEEEEE |
| 359 | E | 0.937 | 0.95121951 | 1096 | # EEEEEEEEEEEEEEEEEEEEEEEEEEEEEEEEEEEDEEDEE |
| 360 | L | 1 | 1 | 2516 | # LLLLLLLLLLLLLLLLLLLLLLLLLLLLLLLLLLLLLLLLL |
| 361 | R | 1 | 1 | 2517 | # RRRRRRRRRRRRRRRRRRRRRRRRRRRRRRRRRRRRRRRRR |
| 362 | Q | 0.803 | 0.85365854 | 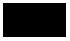 | # QEAQQQHQQQRQQQQQQQQQQQQQQEQRQQQQQQQQQQQQQ |
| 363 | H | 0.963 | 0.97560976 | 1422 | # YHHHHHHHHHHHHHHHHHHHHHHHHHHHHHHHHHHHHHHHH |
| 364 | L | 1 | 1 | 2518 | # LLLLLLLLLLLLLLLLLLLLLLLLLLLLLLLLLLLLLLLLL |
| 365 | L | 1 | 1 | 2519 | # LLLLLLLLLLLLLLLLLLLLLLLLLLLLLLLLLLLLLLLLL |
| 366 | R | 0.797 | 0.68292683 | 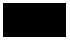 | # KKKRKRRRKKRRKRRKRRRRRRRRRKRRRRKRRRRKRRKRK |
| 367 | W | 0.963 | 0.97560976 | 1423 | # WWWW$WWWWWWWWWWWWWWWWWWWWWWWWWWWWWWWWWWWW |
| 368 | G | 1 | 1 | 2520 | # GGGGGGGGGGGGGGGGGGGGGGGGGGGGGGGGGGGGGGGGG |
| 369 | F | 0.852 | 0.82926829 | 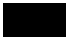 | # FFFLLFFFFFFFLFFFLLFLFFFFFFFFFFFFFFFFFLFFF |
| 370 | T | 0.853 | 0.90243902 | 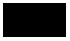 | # TTTTTTTTTTTTTTTTTTTTSTTTITTTTTTTTTTTTFYTT |
| 371 | T | 1 | 1 | 2521 | # TTTTTTTTTTTTTTTTTTTTTTTTTTTTTTTTTTTTTTTTT |
| 372 | P | 1 | 1 | 2522 | # PPPPPPPPPPPPPPPPPPPPPPPPPPPPPPPPPPPPPPPPP |
| 373 | D | 1 | 1 | 2523 | # DDDDDDDDDDDDDDDDDDDDDDDDDDDDDDDDDDDDDDDDD |
| 374 | K | 0.963 | 0.97560976 | 1424 | # KKKKKKKKKKKKKKKKKKKKKKKKKKKKKKKKKKKKKQKKK |
| 375 | K | 1 | 1 | 2524 | # KKKKKKKKKKKKKKKKKKKKKKKKKKKKKKKKKKKKKKKKK |
| 376 | H | 1 | 1 | 2525 | # HHHHHHHHHHHHHHHHHHHHHHHHHHHHHHHHHHHHHHHHH |
| 377 | Q | 1 | 1 | 2526 | # QQQQQQQQQQQQQQQQQQQQQQQQQQQQQQQQQQQQQQQQQ |
| 378 | K | 1 | 1 | 2527 | # KKKKKKKKKKKKKKKKKKKKKKKKKKKKKKKKKKKKKKKKK |
| 379 | E | 0.963 | 0.97560976 | 1425 | # EEEEEEEEEEEEEEEEEEEEEEEEEEEEEEEEEEEEDEEEE |

Table 2I

| | | | | | |
|---|---|---|---|---|---|
| 380 | P | 1 | 1 | 2528 | # PPPPPPPPPPPPPPPPPPPPPPPPPPPPPPPPPPPPPPPPP |
| 381 | P | 1 | 1 | 2529 | # PPPPPPPPPPPPPPPPPPPPPPPPPPPPPPPPPPPPPPPPP |
| 382 | F | 1 | 1 | 2530 | # FFFFFFFFFFFFFFFFFFFFFFFFFFFFFFFFFFFFFFFFF |
| 383 | L | 1 | 1 | 2531 | # LLLLLLLLLLLLLLLLLLLLLLLLLLLLLLLLLLLLLLLLL |
| 384 | W | 1 | 1 | 2532 | # WWWWWWWWWWWWWWWWWWWWWWWWWWWWWWWWWWWWWWWWW |
| 385 | M | 0.963 | 0.97560976 | 1426 | # MMMMIMMMMMMMMMMMMMMMMMMMMMMMMMMMMMMMMMMMM |
| 386 | G | 1 | 1 | 2533 | # GGGGGGGGGGGGGGGGGGGGGGGGGGGGGGGGGGGGGGGGG |
| 387 | Y | 1 | 1 | 2534 | # YYYYYYYYYYYYYYYYYYYYYYYYYYYYYYYYYYYYYYYYY |
| 388 | E | 1 | 1 | 2535 | # EEEEEEEEEEEEEEEEEEEEEEEEEEEEEEEEEEEEEEEEE |
| 389 | L | 1 | 1 | 2536 | # LLLLLLLLLLLLLLLLLLLLLLLLLLLLLLLLLLLLLLLLL |
| 390 | H | 1 | 1 | 2537 | # HHHHHHHHHHHHHHHHHHHHHHHHHHHHHHHHHHHHHHHHH |
| 391 | P | 0.963 | 0.97560976 | 1427 | # PPPPPPPPPPPPPLPPPPPPPPPPPPPPPPPPPPPPPPPPP |
| 392 | D | 0.963 | 0.97560976 | 1428 | # DDDDDDDDDDDNDDDDDDDDDDDDDDDDDDDDDDDDDDDDD |
| 393 | K | 1 | 1 | 2538 | # KKKKKKKKKKKKKKKKKKKKKKKKKKKKKKKKKKKKKKKKK |
| 394 | W | 1 | 1 | 2539 | # WWWWWWWWWWWWWWWWWWWWWWWWWWWWWWWWWWWWWWWWW |
| 395 | T | 1 | 1 | 2540 | # TTTTTTTTTTTTTTTTTTTTTTTTTTTTTTTTTTTTTTTTT |
| 396 | V | 1 | 1 | 2541 | # VVVVVVVVVVVVVVVVVVVVVVVVVVVVVVVVVVVVVVVVV |
| 397 | Q | 1 | 1 | 2542 | # QQQQQQQQQQQQQQQQQQQQQQQQQQQQQQQQQQQQQQQQQ |
| 398 | P | 1 | 1 | 2543 | # PPPPPPPPPPPPPPPPPPPPPPPPPPPPPPPPPPPPPPPPP |
| 399 | I | 1 | 1 | 2544 | # IIIIIIIIIIIIIIIIIIIIIIIIIIIIIIIIIIIIIIIII |
| 400 | V | 0.784 | 0.80487805 | 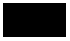 | # TMVVVVVVVVVVVMTVVVMMVVVVVVMVVVVVVVVVKVVVV |
| 401 | L | 1 | 1 | 2545 | # LLLLLLLLLLLLLLLLLLLLLLLLLLLLLLLLLLLLLLLLL |
| 402 | P | 1 | 1 | 2546 | # PPPPPPPPPPPPPPPPPPPPPPPPPPPPPPPPPPPPPPPPP |
| 403 | E | 0.963 | 0.97560976 | 1429 | # EEEEEEEEEEEEHEEEEEEEEEEEEEEEEEEEEEEEEEEEE |
| 404 | K | 1 | 1 | 2547 | # KKKKKKKKKKKKKKKKKKKKKKKKKKKKKKKKKKKKKKKKK |
| 405 | D | 0.963 | 0.97560976 | 1430 | # DDDDDDDDDDDDDDDDDDDDDDDDDDDDDDDDDDDDEDDDD |
| 406 | S | 1 | 1 | 2548 | # SSSSSSSSSSSSSSSSSSSSSSSSSSSSSSSSSSSSSSSSS |
| 407 | W | 0.963 | 0.97560976 | 1431 | # WWWW$WWWWWWWWWWWWWWWWWWWWWWWWWWWWWWWWWWWW |
| 408 | T | 1 | 1 | 2549 | # TTTTTTTTTTTTTTTTTTTTTTTTTTTTTTTTTTTTTTTTT |
| 409 | V | 1 | 1 | 2550 | # VVVVVVVVVVVVVVVVVVVVVVVVVVVVVVVVVVVVVVVVV |
| 410 | N | 1 | 1 | 2551 | # NNNNNNNNNNNNNNNNNNNNNNNNNNNNNNNNNNNNNNNNN |
| 411 | D | 1 | 1 | 2552 | # DDDDDDDDDDDDDDDDDDDDDDDDDDDDDDDDDDDDDDDDD |
| 412 | I | 1 | 1 | 2553 | # IIIIIIIIIIIIIIIIIIIIIIIIIIIIIIIIIIIIIIIII |
| 413 | Q | 1 | 1 | 2554 | # QQQQQQQQQQQQQQQQQQQQQQQQQQQQQQQQQQQQQQQQQ |
| 414 | K | 1 | 1 | 2555 | # KKKKKKKKKKKKKKKKKKKKKKKKKKKKKKKKKKKKKKKKK |
| 415 | L | 1 | 1 | 2556 | # LLLLLLLLLLLLLLLLLLLLLLLLLLLLLLLLLLLLLLLLL |
| 416 | V | 1 | 1 | 2557 | # VVVVVVVVVVVVVVVVVVVVVVVVVVVVVVVVVVVVVVVVV |
| 417 | G | 1 | 1 | 2558 | # GGGGGGGGGGGGGGGGGGGGGGGGGGGGGGGGGGGGGGGGG |
| 418 | K | 1 | 1 | 2559 | # KKKKKKKKKKKKKKKKKKKKKKKKKKKKKKKKKKKKKKKKK |
| 419 | L | 1 | 1 | 2560 | # LLLLLLLLLLLLLLLLLLLLLLLLLLLLLLLLLLLLLLLLL |
| 420 | N | 0.963 | 0.97560976 | 1432 | # NNNNNNNNNNNYNNNNNNNNNNNNNNNNNNNNNNNNNNNNN |
| 421 | W | 0.963 | 0.97560976 | 1433 | # WWWW$WWWWWWWWWWWWWWWWWWWWWWWWWWWWWWWWWWWW |
| 422 | A | 1 | 1 | 2561 | # AAAAAAAAAAAAAAAAAAAAAAAAAAAAAAAAAAAAAAAAA |
| 423 | S | 1 | 1 | 2562 | # SSSSSSSSSSSSSSSSSSSSSSSSSSSSSSSSSSSSSSSSS |
| 424 | Q | 1 | 1 | 2563 | # QQQQQQQQQQQQQQQQQQQQQQQQQQQQQQQQQQQQQQQQQ |
| 425 | I | 1 | 1 | 2564 | # IIIIIIIIIIIIIIIIIIIIIIIIIIIIIIIIIIIIIIIII |
| 426 | Y | 0.963 | 0.97560976 | 1434 | # YYYFYYYYYYYYYYYYYYYYYYYYYYYYYYYYYYYYYYYYY |
| 427 | A | 0.735 | 0.6097561 | 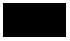 | # PPPPPSPAPAPAPAAAPPAAPAAAAAAAAAAAASAAAAPAP |

Table 2J

| | | | | | |
|---|---|---|---|---|---|
| 428 | G | 0.963 | 0.97560976 | 1435 | # GGGGKGGGGGGGGGGGGGGGGGGGGGGGGGGGGGGGGGGGG |
| 429 | I | 1 | 1 | 2565 | # IIIIIIIIIIIIIIIIIIIIIIIIIIIIIIIIIIIIIIIII |
| 430 | K | 0.963 | 0.97560976 | 1436 | # KKKKKKKKKKKRKKKKKKKKKKKKKKKKKKKKKKKKKKKKK |
| 431 | V | 1 | 1 | 2566 | # VVVVVVVVVVVVVVVVVVVVVVVVVVVVVVVVVVVVVVVVV |
| 432 | K | 0.797 | 0.68292683 | 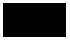 | # KKKRRRKKRKKKKKRKRRKKRKRRRKKKRKKKRKKKKKKKK |
| 433 | Q | 0.864 | 0.09243902 | 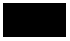 | # QQQQQQQQQQQQQEQQQQNQQQHQQQQQQQQQQQQQQQHQQ |
| 434 | L | 1 | 1 | 2567 | # LLLLLLLLLLLLLLLLLLLLLLLLLLLLLLLLLLLLLLLLL |
| 435 | C | 1 | 1 | 2568 | # CCCCCCCCCCCCCCCCCCCCCCCCCCCCCCCCCCCCCCCCC |
| 436 | K | 1 | 1 | 2569 | # KKKKKKKKKKKKKKKKKKKKKKKKKKKKKKKKKKKKKKKKK |
| 437 | L | 1 | 1 | 2570 | # LLLLLLLLLLLLLLLLLLLLLLLLLLLLLLLLLLLLLLLLL |
| 438 | L | 0.963 | 0.97560976 | 1437 | # LLLLLLLILLLLLLLLLLLLLLLLLLLLLLLLLLLLLLLLL |
| 439 | R | 1 | 1 | 2571 | # RRRRRRRRRRRRRRRRRRRRRRRRRRRRRRRRRRRRRRRRR |
| 440 | G | 1 | 1 | 2572 | # GGGGGGGGGGGGGGGGGGGGGGGGGGGGGGGGGGGGGGGGG |
| 441 | T | 0.808 | 0.82926829 | 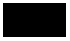 | # ATTTTTTTTTTATTAPTTTTATTTTTTTITTTTTTTTATTT |
| 442 | K | 1 | 1 | 2573 | # KKKKKKKKKKKKKKKKKKKKKKKKKKKKKKKKKKKKKKKKK |
| 443 | A | 1 | 1 | 2574 | # AAAAAAAAAAAAAAAAAAAAAAAAAAAAAAAAAAAAAAAAA |
| 444 | L | 1 | 1 | 2575 | # LLLLLLLLLLLLLLLLLLLLLLLLLLLLLLLLLLLLLLLLL |
| 445 | T | 1 | 1 | 2576 | # TTTTTTTTTTTTTTTTTTTTTTTTTTTTTTTTTTTTTTTTT |
| 446 | E | 1 | 1 | 2577 | # EEEEEEEEEEEEEEEEEEEEEEEEEEEEEEEEEEEEEEEEE |
| 447 | V | 0.937 | 0.95121951 | 1097 | # VVVVVVVIVVVVVVVVVVVVVVVVVVVVVVVVVVVVIVVVV |
| 448 | I | 0.778 | 0.58536585 | 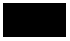 | # IVVVIIVVIIIVIIIIIIIVIVIVVVIIVVIIIVVVIIVII |
| 449 | P | 0.901 | 0.92682927 | 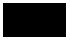 | # PPPPPPPPPPPPPPPPPPPPTPPPPQPPPPPPPTPPPPPPP |
| 450 | L | 0.963 | 0.97560976 | 1438 | # LLLLLLLLLLLLLLLLLLLLLLLLLLLLLLLLLLLLILLLL |
| 451 | T | 1 | 1 | 2578 | # TTTTTTTTTTTTTTTTTTTTTTTTTTTTTTTTTTTTTTTTT |
| 452 | E | 0.77 | 0.75609756 | 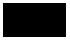 | # KEEEAEKKEEEEEEKKEEEEEEEKEKEEEEKEEEEEEEEER |
| 453 | E | 0.963 | 0.97560976 | 1439 | # EEEEEEEEEEEEEEEEEEEEEEEEEEEEEEEEEEEEEEEEG |
| 454 | A | 1 | 1 | 2579 | # AAAAAAAAAAAAAAAAAAAAAAAAAAAAAAAAAAAAAAAAA |
| 455 | E | 1 | 1 | 2580 | # EEEEEEEEEEEEEEEEEEEEEEEEEEEEEEEEEEEEEEEEE |
| 456 | L | 1 | 1 | 2581 | # LLLLLLLLLLLLLLLLLLLLLLLLLLLLLLLLLLLLLLLLL |
| 457 | E | 1 | 1 | 2582 | # EEEEEEEEEEEEEEEEEEEEEEEEEEEEEEEEEEEEEEEEE |
| 458 | L | 1 | 1 | 2583 | # LLLLLLLLLLLLLLLLLLLLLLLLLLLLLLLLLLLLLLLLL |
| 459 | A | 1 | 1 | 2584 | # AAAAAAAAAAAAAAAAAAAAAAAAAAAAAAAAAAAAAAAAA |
| 460 | E | 0.963 | 0.97560976 | 1440 | # EEEEEEEEEEDEEEEEEEEEEEEEEEEEEEEEEEEEEEEEE |
| 461 | N | 1 | 1 | 2585 | # NNNNNNNNNNNNNNNNNNNNNNNNNNNNNNNNNNNNNNNNN |
| 462 | R | 1 | 1 | 2586 | # RRRRRRRRRRRRRRRRRRRRRRRRRRRRRRRRRRRRRRRRR |
| 463 | E | 1 | 1 | 2587 | # EEEEEEEEEEEEEEEEEEEEEEEEEEEEEEEEEEEEEEEEE |
| 464 | I | 1 | 1 | 2588 | # IIIIIIIIIIIIIIIIIIIIIIIIIIIIIIIIIIIIIIIII |
| 465 | L | 1 | 1 | 2589 | # LLLLLLLLLLLLLLLLLLLLLLLLLLLLLLLLLLLLLLLLL |
| 466 | K | 0.937 | 0.95121951 | 1098 | # KKKKKKKKRKKRKKKKKKKKKKKKKKKKKKKKKKKKKKKKK |
| 467 | E | 0.89 | 0.92682927 | 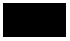 | # VEEEEEEEEEEKEEEEEEEEEEEQEEEEEEEEEEEEEEEEE |
| 468 | P | 0.963 | 0.97560976 | 1441 | # PPPPPPPPPPPPPSPPPPPPPPPPPPPPPPPPPPPPPPPPP |
| 469 | V | 0.963 | 0.97560976 | 1442 | # VVVVIVVVVVVVVVVVVVVVVVVVVVVVVVVVVVVVVVVVV |
| 470 | H | 1 | 1 | 2590 | # HHHHHHHHHHHHHHHHHHHHHHHHHHHHHHHHHHHHHHHHH |
| 471 | G | 0.937 | 0.95121951 | 1099 | # GGGGGGGGGGGEGGGGGGGGGGGGGGEGGGGGGGGGGGGGG |
| 472 | V | 0.963 | 0.97560976 | 1443 | # VVVVVVVVVVVVVVVVVVVVVVVAVVVVVVVVVVVVVVVVV |
| 473 | Y | 1 | 1 | 2591 | # YYYYYYYYYYYYYYYYYYYYYYYYYYYYYYYYYYYYYYYYY |
| 474 | Y | 0.963 | 0.97560976 | 1444 | # YYYYYYYYYYYYYYYCYYYYYYYYYYYYYYYYYYYYYYYYY |
| 475 | D | 1 | 1 | 2592 | # DDDDDDDDDDDDDDDDDDDDDDDDDDDDDDDDDDDDDDDDD |

Table 2K

| | | | | | |
|---|---|---|---|---|---|
| 476 | P | 1 | 1 | 2593 | # PPPPPPPPPPPPPPPPPPPPPPPPPPPPPPPPPPPPPPPPP |
| 477 | S | 0.901 | 0.92682927 | 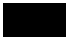 | # SSTSSSSSSSSSSSSSSSSSSASSSSSSSSSSSSSTSSSSS |
| 478 | K | 0.963 | 0.97560976 | 1445 | # KKKKKKKKKKEKKKKKKKKKKKKKKKKKKKKKKKKKKKKKK |
| 479 | D | 0.963 | 0.97560976 | 1446 | # DDDDDDDDDDDDDDDDDDDDDDEDDDDDDDDDDDDDDDDDD |
| 480 | L | 0.963 | 0.97560976 | 1447 | # LLLLLLLLLLLLLLLLLLLLLLLLLLLLLLLLLLLLLILLL |
| 481 | I | 0.794 | 0.75609756 | 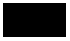 | # TVIIVIIIIIIIIIIVIIVIVIIVIIVIIVIIIIVIIIIII |
| 482 | A | 0.963 | 0.97560976 | 1448 | # AAAAAAAAAAAAAAAAAAAAAAAAAAAAAAVAAAAAAAAAA |
| 483 | E | 0.963 | 0.97560976 | 1449 | # EEEEEEEEEEEEEEEEEEEEEEEEEEEEEEEEEEEEEEEED |
| 484 | I | 0.72 | 0.65853659 | 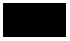 | # IVIIIVIIVIIILIIIIILLIIIIIIIVIVILIILLLILLI |
| 485 | Q | 1 | 1 | 2594 | # QQQQQQQQQQQQQQQQQQQQQQQQQQQQQQQQQQQQQQQQQ |
| 486 | K | 1 | 1 | 2595 | # KKKKKKKKKKKKKKKKKKKKKKKKKKKKKKKKKKKKKKKKK |
| 487 | Q | 1 | 1 | 2596 | # QQQQQQQQQQQQQQQQQQQQQQQQQQQQQQQQQQQQQQQQQ |
| 488 | G | 0.89 | 0.92682927 | 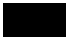 | # GDGGREGGGGGGGGGGGGGGGGGGGGGGGGGGGGGGGGGGG |
| 489 | Q | 0.748 | 0.7804878 | 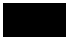 | # QNQEYQQQEQQQELQEQQQQLQQQQQQQQQQQQQQQQQEQQ |
| 490 | G | 0.963 | 0.97560976 | 1450 | # GGGGGGGGGGGGGGGGGGGGGGGGGGGGGGGGGGGGGGGGD |
| 491 | Q | 1 | 1 | 2597 | # QQQQQQQQQQQQQQQQQQQQQQQQQQQQQQQQQQQQQQQQQ |
| 492 | W | 0.963 | 0.97560976 | 1451 | # WWWW$WWWWWWWWWWWWWWWWWWWWWWWWWWWWWWWWWWWW |
| 493 | T | 1 | 1 | 2598 | # TTTTTTTTTTTTTTTTTTTTTTTTTTTTTTTTTTTTTTTTT |
| 494 | Y | 1 | 1 | 2599 | # YYYYYYYYYYYYYYYYYYYYYYYYYYYYYYYYYYYYYYYYY |
| 495 | Q | 1 | 1 | 2600 | # QQQQQQQQQQQQQQQQQQQQQQQQQQQQQQQQQQQQQQQQQ |
| 496 | I | 1 | 1 | 2601 | # IIIIIIIIIIIIIIIIIIIIIIIIIIIIIIIIIIIIIIIII |
| 497 | Y | 0.937 | 0.95121951 | 1100 | # YYYYYYYYYYYYFYYYYYYYYYYYYYYYYYFYYYYYYYYYY |
| 498 | Q | 1 | 1 | 2602 | # QQQQQQQQQQQQQQQQQQQQQQQQQQQQQQQQQQQQQQQQQ |
| 499 | E | 0.937 | 0.95121951 | 1101 | # EEEEEEEEDEEEDEEEEEEEEEEEEEEEEEEEEEEEEEEEE |
| 500 | P | 0.879 | 0.90243902 | 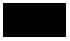 | # PPPQPPPPQPPPPPPPPPPPPPPTPPPPPPPPPPPPPPQPP |
| 501 | F | 0.915 | 0.92682927 | 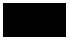 | # FFFFFFFFFFFFFYFFFFFFFFFFFFFFFFFFFFFYFFYFF |
| 502 | K | 0.937 | 0.95121951 | 1102 | # RKKKKKKKRKKKKKKKKKKKKKKKKKKKKKKKKKKKKKKKK |
| 503 | N | 0.926 | 0.95121951 | 969 | # SNNNNNNNNNNNNNNNNNNNNNNNNNNNNNNINNNNNNNNN |
| 504 | L | 1 | 1 | 2603 | # LLLLLLLLLLLLLLLLLLLLLLLLLLLLLLLLLLLLLLLLL |
| 505 | K | 1 | 1 | 2604 | # KKKKKKKKKKKKKKKKKKKKKKKKKKKKKKKKKKKKKKKKK |
| 506 | T | 1 | 1 | 2605 | # TTTTTTTTTTTTTTTTTTTTTTTTTTTTTTTTTTTTTTTTT |
| 507 | G | 0.963 | 0.97560976 | 1452 | # GGGGGGGGGGGGGGGGGGGGGGGGGGGGGGGGGAGGGGGGG |
| 508 | K | 1 | 1 | 2606 | # KKKKKKKKKKKKKKKKKKKKKKKKKKKKKKKKKKKKKKKKK |
| 509 | Y | 1 | 1 | 2607 | # YYYYYYYYYYYYYYYYYYYYYYYYYYYYYYYYYYYYYYYYY |
| 510 | A | 0.963 | 0.97560976 | 1453 | # AAAAAAAAAAAAAAAAAAAAAAAAAAAAAAAAAAAAAAATA |
| 511 | R | 0.852 | 0.82926829 | 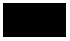 | # KRRKRRRRRRRKRRRRRRRKKRRRRRRRRKRRRRRRRRRRK |
| 512 | M | 0.727 | 0.68292683 | 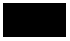 | # MMMMMMMTMVMTAMMMMMMMMMMTMMMMMMTTTTTMVTMMT |
| 513 | R | 0.897 | 0.90243902 | 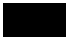 | # RRRRRKRRRRRRKRRRRRRRRRRRKRRRKRRRRRRRRRRRR |
| 514 | G | 1 | 1 | 2608 | # GGGGGGGGGGGGGGGGGGGGGGGGGGGGGGGGGGGGGGGGG |
| 515 | A | 0.865 | 0.85365854 | 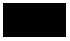 | # TAATTAAAAAAAAAAAAAATAAAAAAAAAAAAAAAAAATAT |
| 516 | H | 1 | 1 | 2609 | # HHHHHHHHHHHHHHHHHHHHHHHHHHHHHHHHHHHHHHHHH |
| 517 | T | 1 | 1 | 2610 | # TTTTTTTTTTTTTTTTTTTTTTTTTTTTTTTTTTTTTTTTT |
| 518 | N | 1 | 1 | 2611 | # NNNNNNNNNNNNNNNNNNNNNNNNNNNNNNNNNNNNNNNNN |
| 519 | D | 1 | 1 | 2612 | # DDDDDDDDDDDDDDDDDDDDDDDDDDDDDDDDDDDDDDDDD |
| 520 | V | 0.937 | 0.95121951 | 1103 | # VVVVVVVVVVVVIVVIVVVVVVVVVVVVVVVVVVVVVVVVV |
| 521 | K | 0.937 | 0.95121951 | 1104 | # KKRKKKKKKKRKKKKKKKKKKKKKKKKKKKKKKKKKKKKKK |
| 522 | Q | 1 | 1 | 2613 | # QQQQQQQQQQQQQQQQQQQQQQQQQQQQQQQQQQQQQQQQQ |
| 523 | L | 1 | 1 | 2614 | # LLLLLLLLLLLLLLLLLLLLLLLLLLLLLLLLLLLLLLLLL |

Table 2L

| | | | | | |
|---|---|---|---|---|---|
| 524 | T | 1 | 1 | 2615 | # TTTTTTTTTTTTTTTTTTTTTTTTTTTTTTTTTTTTTTTTT |
| 525 | E | 1 | 1 | 2616 | # EEEEEEEEEEEEEEEEEEEEEEEEEEEEEEEEEEEEEEEEE |
| 526 | A | 0.963 | 0.97560976 | 1454 | # AAAAAAAAAAAAAAAAAAAAAAAAAAAAAAAAAAAAAVAAA |
| 527 | V | 1 | 1 | 2617 | # VVVVVVVVVVVVVVVVVVVVVVVVVVVVVVVVVVVVVVVVV |
| 528 | Q | 1 | 1 | 2618 | # QQQQQQQQQQQQQQQQQQQQQQQQQQQQQQQQQQQQQQQQQ |
| 529 | K | 1 | 1 | 2619 | # KKKKKKKKKKKKKKKKKKKKKKKKKKKKKKKKKKKKKKKKK |
| 530 | I | 0.852 | 0.82926829 | 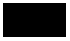 | # VIIIIIIIIIIIIIIIIIIIIVIIIVVIIIIIIIIIIVIVV |
| 531 | A | 0.694 | 0.6097561 | 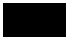 | # TATVSATTASAAAATATTTAAATAAASAAAAAATAATTAAA |
| 532 | T | 0.828 | 0.87804878 | 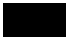 | # TTTTTTTTTTTTTTTTTTQTTITTTTTTTTNNTTTTTTTTM |
| 533 | E | 1 | 1 | 2620 | # EEEEEEEEEEEEEEEEEEEEEEEEEEEEEEEEEEEEEEEEE |
| 534 | S | 0.901 | 0.92682927 | 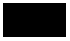 | # SSSSSSSSSSSSSSSGSSSSSSSSSSSSSSSSSCSSSCSSS |
| 535 | I | 1 | 1 | 2621 | # IIIIIIIIIIIIIIIIIIIIIIIIIIIIIIIIIIIIIIIII |
| 536 | V | 0.915 | 0.92682927 | 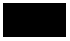 | # IVVVVVVVVIVVVVVVVVVVVVVVVVVVVVVVVIVVVVVVV |
| 537 | I | 1 | 1 | 2622 | # IIIIIIIIIIIIIIIIIIIIIIIIIIIIIIIIIIIIIIIII |
| 538 | W | 0.963 | 0.97560976 | 1455 | # WWWW$WWWWWWWWWWWWWWWWWWWWWWWWWWWWWWWWWWWW |
| 539 | G | 0.963 | 0.97560976 | 1456 | # GGGGGGGGGGGKGGGGGGGGGGGGGGGGGGGGGGGGGGGGG |
| 540 | K | 1 | 1 | 2623 | # KKKKKKKKKKKKKKKKKKKKKKKKKKKKKKKKKKKKKKKKK |
| 541 | T | 0.83 | 0.82926829 | 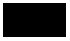 | # IVTITTTITTTTTTTTTTTTTTTTTTITTTIITTTTTTTTT |
| 542 | P | 1 | 1 | 2624 | # PPPPPPPPPPPPPPPPPPPPPPPPPPPPPPPPPPPPPPPPP |
| 543 | K | 1 | 1 | 2625 | # KKKKKKKKKKKKKKKKKKKKKKKKKKKKKKKKKKKKKKKKK |
| 544 | F | 1 | 1 | 2626 | # FFFFFFFFFFFFFFFFFFFFFFFFFFFFFFFFFFFFFFFFF |
| 545 | K | 0.829 | 0.7804878 | 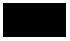 | # KKRRKKRKRKKKKKKRKKKKKKKKKKKRKKKKKRKKKRRKK |
| 546 | L | 1 | 1 | 2627 | # LLLLLLLLLLLLLLLLLLLLLLLLLLLLLLLLLLLLLLLLL |
| 547 | P | 1 | 1 | 2628 | # PPPPPPPPPPPPPPPPPPPPPPPPPPPPPPPPPPPPPPPPP |
| 548 | I | 1 | 1 | 2629 | # IIIIIIIIIIIIIIIIIIIIIIIIIIIIIIIIIIIIIIIII |
| 549 | Q | 1 | 1 | 2630 | # QQQQQQQQQQQQQQQQQQQQQQQQQQQQQQQQQQQQQQQQQ |
| 550 | K | 0.963 | 0.97560976 | 1457 | # KKKKKKKKKRKKKKKKKKKKKKKKKKKKKKKKKKKKKKKKK |
| 551 | E | 1 | 1 | 2631 | # EEEEEEEEEEEEEEEEEEEEEEEEEEEEEEEEEEEEEEEEE |
| 552 | T | 1 | 1 | 2632 | # TTTTTTTTTTTTTTTTTTTTTTTTTTTTTTTTTTTTTTTTT |
| 553 | W | 0.963 | 0.97560976 | 1458 | # WWWW$WWWWWWWWWWWWWWWWWWWWWWWWWWWWWWWWWWWW |
| 554 | E | 0.897 | 0.90243902 | 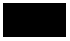 | # DEEEEEEEDEEEEEEEEEEDEEEEEEEEEEEEEEEEEEDEE |
| 555 | A | 0.749 | 0.58536585 | 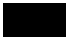 | # AATTAATTAAAATAAATTAAAAAAAAATAATTTTSATTATT |
| 556 | W | 1 | 1 | 2633 | # WWWWWWWWWWWWWWWWWWWWWWWWWWWWWWWWWWWWWWWWW |
| 557 | W | 0.963 | 0.97560976 | 1459 | # WWWW$WWWWWWWWWWWWWWWWWWWWWWWWWWWWWWWWWWWW |
| 558 | T | 0.808 | 0.82926829 | 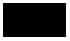 | # TMTTTMTTTTTTTTTTTTTIMTTTTTMTTMTTTTTTTTATT |
| 559 | E | 0.875 | 0.90243902 | 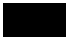 | # EEEEKEEEEEEKEEEEEEEDEEEEEEEEEEEEEEEDEEEEE |
| 560 | Y | 1 | 1 | 2634 | # YYYYYYYYYYYYYYYYYYYYYYYYYYYYYYYYYYYYYYYYY |
| 561 | W | 0.963 | 0.97560976 | 1460 | # WWWWWWWWWWWWWWWWWWWWWWWWWWWTWWWWWWWWWWWWW |
| 562 | Q | 0.963 | 0.97560976 | 1461 | # QQQQQQQQQQQQQQQQQQQQQQQQQQQ$QQQQQQQQQQQQQ |
| 563 | A | 1 | 1 | 2635 | # AAAAAAAAAAAAAAAAAAAAAAAAAAAAAAAAAAAAAAAAA |
| 564 | T | 1 | 1 | 2636 | # TTTTTTTTTTTTTTTTTTTTTTTTTTTTTTTTTTTTTTTTT |
| 565 | W | 0.963 | 0.97560976 | 1462 | # WWWW$WWWWWWWWWWWWWWWWWWWWWWWWWWWWWWWWWWWW |
| 565 | I | 1 | 1 | 2637 | # IIIIIIIIIIIIIIIIIIIIIIIIIIIIIIIIIIIIIIIII |
| 567 | P | 1 | 1 | 2638 | # PPPPPPPPPPPPPPPPPPPPPPPPPPPPPPPPPPPPPPPPP |
| 568 | E | 0.963 | 0.97560976 | 1463 | # EEEEEEEEEEEKEEEEEEEEEEEEEEEEEEEEEEEEEEEEE |
| 569 | W | 0.963 | 0.97560976 | 1464 | # WWWW$WWWWWWWWWWWWWWWWWWWWWWWWWWWWWWWWWWWW |
| 570 | E | 1 | 1 | 2639 | # EEEEEEEEEEEEEEEEEEEEEEEEEEEEEEEEEEEEEEEEE |
| 571 | F | 0.963 | 0.97560976 | 1465 | # FFFFFFFFFFFFFFFFFFFFFFFFFFFVFFFFFFFFFFFFF |

Table 2M

| | | | | | |
|---|---|---|---|---|---|
| 572 | V | 0.963 | 0.97560976 | 1466 | # VVVVVVVVVVVVVIVVVVVVVVVVVVVVVVVVVVVVVVVVV |
| 573 | N | 1 | 1 | 2640 | # NNNNNNNNNNNNNNNNNNNNNNNNNNNNNNNNNNNNNNNNN |
| 574 | T | 1 | 1 | 2641 | # TTTTTTTTTTTTTTTTTTTTTTTTTTTTTTTTTTTTTTTTT |
| 575 | P | 0.963 | 0.97560976 | 1467 | # PPPPPPPPPPPPPSPPPPPPPPPPPPPPPPPPPPPPPPPPP |
| 576 | P | 1 | 1 | 2642 | # PPPPPPPPPPPPPPPPPPPPPPPPPPPPPPPPPPPPPPPPP |
| 577 | L | 1 | 1 | 2643 | # LLLLLLLLLLLLLLLLLLLLLLLLLLLLLLLLLLLLLLLLL |
| 578 | V | 1 | 1 | 2644 | # VVVVVVVVVVVVVVVVVVVVVVVVVVVVVVVVVVVVVVVVV |
| 579 | K | 1 | 1 | 2645 | # KKKKKKKKKKKKKKKKKKKKKKKKKKKKKKKKKKKKKKKKK |
| 580 | L | 1 | 1 | 2646 | # LLLLLLLLLLLLLLLLLLLLLLLLLLLLLLLLLLLLLLLLL |
| 581 | W | 0.963 | 0.97560976 | 1468 | # WWWW$WWWWWWWWWWWWWWWWWWWWWWWWWWWWWWWWWWWW |
| 582 | Y | 1 | 1 | 2647 | # YYYYYYYYYYYYYYYYYYYYYYYYYYYYYYYYYYYYYYYYY |
| 583 | Q | 1 | 1 | 2648 | # QQQQQQQQQQQQQQQQQQQQQQQQQQQQQQQQQQQQQQQQQ |
| 584 | L | 1 | 1 | 2649 | # LLLLLLLLLLLLLLLLLLLLLLLLLLLLLLLLLLLLLLLLL |
| 585 | E | 1 | 1 | 2650 | # EEEEEEEEEEEEEEEEEEEEEEEEEEEEEEEEEEEEEEEEE |
| 586 | K | 0.901 | 0.92682927 | 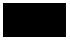 | # KKKKTKKKKKRKKKTKKKKKKKKKKKKKKKKKKKKKKKKKK |
| 587 | E | 0.937 | 0.95121951 | 1105 | # EEEEEEEEEEEEDEEEEEDEEEEEEEEEEEEEEEEEEEEEE |
| 588 | P | 1 | 1 | 2651 | # PPPPPPPPPPPPPPPPPPPPPPPPPPPPPPPPPPPPPPPPP |
| 589 | I | 1 | 1 | 2652 | # IIIIIIIIIIIIIIIIIIIIIIIIIIIIIIIIIIIIIIIII |
| 590 | V | 0.647 | 0.63414634 | 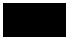 | # VVVVVVVITIIIEEVVVVVVIVVIIIVVIVVVIEVVVVVVL |
| 591 | G | 0.926 | 0.95121951 | 970 | # GGGGRGGGGGGGGEGGGGGGGGGGGGGGGGGGGGGGGGGGG |
| 592 | A | 0.963 | 0.97560976 | 1469 | # AAAAAAAAAAAAVAAAAAAAAAAAAAAAAAAAAAAAAAAAA |
| 593 | E | 1 | 1 | 2653 | # EEEEEEEEEEEEEEEEEEEEEEEEEEEEEEEEEEEEEEEEE |
| 594 | T | 1 | 1 | 2654 | # TTTTTTTTTTTTTTTTTTTTTTTTTTTTTTTTTTTTTTTTT |
| 595 | F | 1 | 1 | 2655 | # FFFFFFFFFFFFFFFFFFFFFFFFFFFFFFFFFFFFFFFFF |
| 596 | Y | 1 | 1 | 2656 | # YYYYYYYYYYYYYYYYYYYYYYYYYYYYYYYYYYYYYYYYY |
| 597 | V | 1 | 1 | 2657 | # VVVVVVVVVVVVVVVVVVVVVVVVVVVVVVVVVVVVVVVVV |
| 598 | D | 0.963 | 0.97560976 | 1470 | # DDDDDDDDDDDNDDDDDDDDDDDDDDDDDDDDDDDDDDDDD |
| 599 | G | 0.963 | 0.97560976 | 1471 | # GGGGRGGGGGGGGGGGGGGGGGGGGGGGGGGGGGGGGGGGG |
| 600 | A | 1 | 1 | 2658 | # AAAAAAAAAAAAAAAAAAAAAAAAAAAAAAAAAAAAAAAAA |
| 601 | A | 1 | 1 | 2659 | # AAAAAAAAAAAAAAAAAAAAAAAAAAAAAAAAAAAAAAAAA |
| 602 | N | 0.963 | 0.97560976 | 1472 | # NNNNNNNNNNNNNNNNNNNNNNNNNNNNNNNNNNNNNSNNN |
| 603 | R | 0.915 | 0.92682927 | 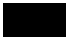 | # RRRRKRRRRRKRRRRRRRRRRRRRRRRRRRRRRRRRKRRRR |
| 604 | E | 0.937 | 0.95121951 | 1106 | # EEEEEEEEEEEEEEEEEEEEEEEEEEEEEEEEEEEDEEEED |
| 605 | T | 1 | 1 | 2660 | # TTTTTTTTTTTTTTTTTTTTTTTTTTTTTTTTTTTTTTTTT |
| 606 | K | 0.937 | 0.95121951 | 1107 | # KKKKKKKKKKKKKKKKKKKRKKKKKKKKKKKKKKKKKRKKK |
| 607 | L | 0.782 | 0.82926829 | 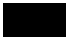 | # LISLLLLLLILVLLLLLLLLLLLLLLLKLLLLLLLSLLLSL |
| 608 | G | 0.926 | 0.95121951 | 971 | # GGGGRGGGGGGKGGGGGGGGGGGGGGGGGGGGGGGGGGGGG |
| 609 | K | 0.963 | 0.97560976 | 1473 | # KKKKKKKKKKKKKKKKKKKKKKKKKKKKKKKKKKKKKKRKK |
| 610 | A | 1 | 1 | 2661 | # AAAAAAAAAAAAAAAAAAAAAAAAAAAAAAAAAAAAAAAAA |
| 611 | G | 1 | 1 | 2662 | # GGGGGGGGGGGGGGGGGGGGGGGGGGGGGGGGGGGGGGGGG |
| 612 | Y | 1 | 1 | 2663 | # YYYYYYYYYYYYYYYYYYYYYYYYYYYYYYYYYYYYYYYYY |
| 613 | V | 1 | 1 | 2664 | # VVVVVVVVVVVVVVVVVVVVVVVVVVVVVVVVVVVVVVVVV |
| 614 | T | 1 | 1 | 2665 | # TTTTTTTTTTTTTTTTTTTTTTTTTTTTTTTTTTTTTTTTT |
| 615 | D | 0.749 | 0.58536585 | 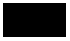 | # DDNDDDNNDNDNNNDDNNDDDDDDDDDNDDSNNNDDNNDDN |
| 616 | R | 0.852 | 0.82926829 | 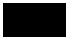 | # RRRRRRRKRKRKRKRRRRRRRRRKRRRRRRRRKKRRRRRRR |
| 617 | G | 1 | 1 | 2666 | # GGGGGGGGGGGGGGGGGGGGGGGGGGGGGGGGGGGGGGGGG |
| 618 | R | 0.963 | 0.97560976 | 1474 | # RRRRRRRRRRRRRRRRRRRRRRRRRRRRRRRRRRRRRRKRR |
| 619 | Q | 1 | 1 | 2667 | # QQQQQQQQQQQQQQQQQQQQQQQQQQQQQQQQQQQQQQQQQ |

Table 2N

| | | | | | |
|---|---|---|---|---|---|
| 620 | K | 0.963 | 0.97560976 | 1475 | # KKKKKKKKKKKKKKKKKKKKKKKKKKKKKKKKKKKKRKKKK |
| 621 | V | 1 | 1 | 2668 | # VVVVVVVVVVVVVVVVVVVVVVVVVVVVVVVVVVVVVVVVV |
| 622 | V | 0.963 | 0.97560976 | 1476 | # IVVVVVVVVVVVVVVVVVVVVVVVVVVVVVVVVVVVVVVVV |
| 623 | S | 0.669 | 0.6097561 | 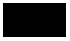 | # SPSSSPPSSLSLSTSSTTSPPPSSPSSSPPSSSTSSSSSPS |
| 624 | L | 0.937 | 0.95121951 | 1108 | # LLLLLLLLLLLLLLLLLLLLLLLLLLILLLLLLLLLLLLIL |
| 625 | T | 0.744 | 0.7804878 | 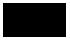 | # TKTTTSTTATTNTTTNTTTTTTNTTTATTTTTTTTATTTAT |
| 626 | D | 1 | 1 | 2669 | # DDDDDDDDDDDDDDDDDDDDDDDDDDDDDDDDDDDDDDDDD |
| 627 | T | 1 | 1 | 2670 | # TTTTTTTTTTTTTTTTTTTTTTTTTTTTTTTTTTTTTTTTT |
| 628 | T | 1 | 1 | 2671 | # TTTTTTTTTTTTTTTTTTTTTTTTTTTTTTTTTTTTTTTTT |
| 629 | N | 1 | 1 | 2672 | # NNNNNNNNNNNNNNNNNNNNNNNNNNNNNNNNNNNNNNNNN |
| 630 | Q | 1 | 1 | 2673 | # QQQQQQQQQQQQQQQQQQQQQQQQQQQQQQQQQQQQQQQQQ |
| 631 | K | 0.963 | 0.97560976 | 1477 | # KKQKKKKKKKKKKKKKKKKKKKKKKKKKKKKKKKKKKKKKK |
| 632 | T | 1 | 1 | 2674 | # TTTTTTTTTTTTTTTTTTTTTTTTTTTTTTTTTTTTTTTTT |
| 633 | E | 1 | 1 | 2675 | # EEEEEEEEEEEEEEEEEEEEEEEEEEEEEEEEEEEEEEEEE |
| 634 | L | 1 | 1 | 2676 | # LLLLLLLLLLLLLLLLLLLLLLLLLLLLLLLLLLLLLLLLL |
| 635 | Q | 0.963 | 0.97560976 | 1478 | # QQQQQQQQQQQQQQQQQQQQQQQQQQQQQQQQQEQQQQQQQ |
| 636 | A | 1 | 1 | 2677 | # AAAAAAAAAAAAAAAAAAAAAAAAAAAAAAAAAAAAAAAAA |
| 637 | I | 1 | 1 | 2678 | # IIIIIIIIIIIIIIIIIIIIIIIIIIIIIIIIIIIIIIIII |
| 638 | H | 0.759 | 0.73170732 | 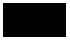 | # HHHHHQHYHHHHYYHHYYHYHHNHHHHHHHHHYHHHHYHHY |
| 639 | L | 1 | 1 | 2679 | # LLLLLLLLLLLLLLLLLLLLLLLLLLLLLLLLLLLLLLLLL |
| 640 | A | 1 | 1 | 2680 | # AAAAAAAAAAAAAAAAAAAAAAAAAAAAAAAAAAAAAAAAA |
| 641 | L | 1 | 1 | 2681 | # LLLLLLLLLLLLLLLLLLLLLLLLLLLLLLLLLLLLLLLLL |
| 642 | Q | 1 | 1 | 2682 | # QQQQQQQQQQQQQQQQQQQQQQQQQQQQQQQQQQQQQQQQQ |
| 643 | D | 1 | 1 | 2683 | # DDDDDDDDDDDDDDDDDDDDDDDDDDDDDDDDDDDDDDDDD |
| 644 | S | 1 | 1 | 2684 | # SSSSSSSSSSSSSSSSSSSSSSSSSSSSSSSSSSSSSSSSS |
| 645 | G | 1 | 1 | 2685 | # GGGGGGGGGGGGGGGGGGGGGGGGGGGGGGGGGGGGGGGGG |
| 646 | L | 0.843 | 0.87804878 | 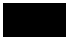 | # LLLLLLLLSLLLPVLLLLLLLLLLSLLLLLLLLLLLLLLLS |
| 647 | E | 1 | 1 | 2686 | # EEEEEEEEEEEEEEEEEEEEEEEEEEEEEEEEEEEEEEEEE |
| 648 | V | 1 | 1 | 2687 | # VVVVVVVVVVVVVVVVVVVVVVVVVVVVVVVVVVVVVVVVV |
| 649 | N | 1 | 1 | 2688 | # NNNNNNNNNNNNNNNNNNNNNNNNNNNNNNNNNNNNNNNNN |
| 650 | I | 1 | 1 | 2689 | # IIIIIIIIIIIIIIIIIIIIIIIIIIIIIIIIIIIIIIIII |
| 651 | V | 1 | 1 | 2690 | # VVVVVVVVVVVVVVVVVVVVVVVVVVVVVVVVVVVVVVVVV |
| 652 | T | 0.937 | 0.95121951 | 1109 | # STTTTTTTTTTTTTSTTTTTTTTTTTTTTTTTTTTTTTTTT |
| 653 | D | 1 | 1 | 2691 | # DDDDDDDDDDDDDDDDDDDDDDDDDDDDDDDDDDDDDDDDD |
| 654 | S | 1 | 1 | 2692 | # SSSSSSSSSSSSSSSSSSSSSSSSSSSSSSSSSSSSSSSSS |
| 655 | Q | 1 | 1 | 2693 | # QQQQQQQQQQQQQQQQQQQQQQQQQQQQQQQQQQQQQQQQQ |
| 656 | Y | 1 | 1 | 2694 | # YYYYYYYYYYYYYYYYYYYYYYYYYYYYYYYYYYYYYYYYY |
| 657 | A | 1 | 1 | 2695 | # AAAAAAAAAAAAAAAAAAAAAAAAAAAAAAAAAAAAAAAAA |
| 658 | L | 0.963 | 0.97560976 | 1479 | # LLLLLLLLLLLLLLILLLLLLLLLLLLLLLLLLLLLLLLLL |
| 659 | G | 1 | 1 | 2696 | # GGGGGGGGGGGGGGGGGGGGGGGGGGGGGGGGGGGGGGGGG |
| 660 | I | 1 | 1 | 2697 | # IIIIIIIIIIIIIIIIIIIIIIIIIIIIIIIIIIIIIIIII |
| 661 | I | 0.963 | 0.97560976 | 1480 | # IIIILIIIIIIIIIIIIIIIIIIIIIIIIIIIIIIIIIIII |
| 662 | Q | 0.963 | 0.97560976 | 1481 | # QQQQQQQQQHQQQQQQQQQQQQQQQQQQQQQQQQQQQQQQQ |
| 663 | A | 1 | 1 | 2698 | # AAAAAAAAAAAAAAAAAAAAAAAAAAAAAAAAAAAAAAAAA |
| 664 | Q | 0.926 | 0.95121951 | 972 | # QQQQTQQQQQQQQQQQQQQQQQQQQQQQQQQQQQQQQQHQQ |
| 665 | P | 1 | 1 | 2699 | # PPPPPPPPPPPPPPPPPPPPPPPPPPPPPPPPPPPPPPPPP |
| 666 | D | 1 | 1 | 2700 | # DDDDDDDDDDDDDDDDDDDDDDDDDDDDDDDDDDDDDDDDD |
| 667 | K | 0.797 | 0.80487805 | 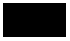 | # KKKKKKKQKKKKRKKKQQKKRKKKKKKKKKKKRKRKQKKKK |

Table 2O

| | | | | | |
|---|---|---|---|---|---|
| 668 | S | 1 | 1 | 2701 | # SSSSSSSSSSSSSSSSSSSSSSSSSSSSSSSSSSSSSSSSS |
| 669 | E | 1 | 1 | 2702 | # EEEEEEEEEEEEEEEEEEEEEEEEEEEEEEEEEEEEEEEEE |
| 670 | S | 1 | 1 | 2703 | # SSSSSSSSSSSSSSSSSSSSSSSSSSSSSSSSSSSSSSSSS |
| 671 | E | 1 | 1 | 2704 | # EEEEEEEEEEEEEEEEEEEEEEEEEEEEEEEEEEEEEEEEE |
| 672 | L | 0.819 | 0.82926829 | 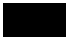 | # LLLLILLLIIVIILLLLLLLVLLLLLLLLLLLLLLLLLLLL |
| 673 | V | 1 | 1 | 2705 | # VVVVVVVVVVVVVVVVVVVVVVVVVVVVVVVVVVVVVVVVV |
| 674 | S | 0.829 | 0.7804878 | 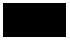 | # SNNSSNSSNSSNSSSSNNSSSSSSSSSSSSSSSSSSSNNSS |
| 675 | Q | 0.963 | 0.97560976 | 1482 | # QQQQQQQQNQQQQQQQQQQQQQQQQQQQQQQQQQQQQQQQQ |
| 676 | I | 1 | 1 | 2706 | # IIIIIIIIIIIIIIIIIIIIIIIIIIIIIIIIIIIIIIIII |
| 677 | I | 1 | 1 | 2707 | # IIIIIIIIIIIIIIIIIIIIIIIIIIIIIIIIIIIIIIIII |
| 678 | E | 1 | 1 | 2708 | # EEEEEEEEEEEEEEEEEEEEEEEEEEEEEEEEEEEEEEEEE |
| 679 | Q | 0.901 | 0.92682927 | 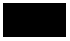 | # QQQLQQQQQQQQQQQQQQQEQQQEQQQQQQQQQQQQQQQQQ |
| 680 | L | 1 | 1 | 2709 | # LLLLLLLLLLLLLLLLLLLLLLLLLLLLLLLLLLLLLLLLL |
| 681 | I | 1 | 1 | 2710 | # IIIIIIIIIIIIIIIIIIIIIIIIIIIIIIIIIIIIIIIII |
| 682 | K | 0.963 | 0.97560976 | 1483 | # KKKKKKKKKKKKKKKKKKKKKKKKEKKKKKKKKKKKKKKKK |
| 683 | K | 1 | 1 | 2711 | # KKKKKKKKKKKKKKKKKKKKKKKKKKKKKKKKKKKKKKKKK |
| 684 | E | 1 | 1 | 2712 | # EEEEEEEEEEEEEEEEEEEEEEEEEEEEEEEEEEEEEEEEE |
| 685 | K | 1 | 1 | 2713 | # KKKKKKKKKKKKKKKKKKKKKKKKKKKKKKKKKKKKKKKKK |
| 686 | V | 0.963 | 0.97560976 | 1484 | # VVVVVIVVVVVVVVVVVVVVVVVVVVVVVVVVVVVVVVVVV |
| 687 | Y | 1 | 1 | 2714 | # YYYYYYYYYYYYYYYYYYYYYYYYYYYYYYYYYYYYYYYYY |
| 688 | L | 1 | 1 | 2715 | # LLLLLLLLLLLLLLLLLLLLLLLLLLLLLLLLLLLLLLLLL |
| 689 | A | 0.937 | 0.95121951 | 1110 | # AAAAAAAAAAAAAATAAAAAAAATAAAAAAAAAAAAAAAAA |
| 690 | W | 0.963 | 0.97560976 | 1485 | # WWWW$WWWWWWWWWWWWWWWWWWWWWWWWWWWWWWWWWWWW |
| 691 | V | 1 | 1 | 2716 | # VVVVVVVVVVVVVVVVVVVVVVVVVVVVVVVVVVVVVVVVV |
| 692 | P | 0.963 | 0.97560976 | 1486 | # PPPPPPPPPPPPPPPPPPPPPPPPPPPPPPPPPPPPPPPLP |
| 693 | A | 1 | 1 | 2717 | # AAAAAAAAAAAAAAAAAAAAAAAAAAAAAAAAAAAAAAAAA |
| 694 | H | 1 | 1 | 2718 | # HHHHHHHHHHHHHHHHHHHHHHHHHHHHHHHHHHHHHHHHH |
| 695 | K | 1 | 1 | 2719 | # KKKKKKKKKKKKKKKKKKKKKKKKKKKKKKKKKKKKKKKKK |
| 696 | G | 1 | 1 | 2720 | # GGGGGGGGGGGGGGGGGGGGGGGGGGGGGGGGGGGGGGGGG |
| 697 | I | 1 | 1 | 2721 | # IIIIIIIIIIIIIIIIIIIIIIIIIIIIIIIIIIIIIIIII |
| 698 | G | 0.963 | 0.97560976 | 1487 | # GGGGRGGGGGGGGGGGGGGGGGGGGGGGGGGGGGGGGGGGG |
| 699 | G | 1 | 1 | 2722 | # GGGGGGGGGGGGGGGGGGGGGGGGGGGGGGGGGGGGGGGGG |
| 700 | N | 1 | 1 | 2723 | # NNNNNNNNNNNNNNNNNNNNNNNNNNNNNNNNNNNNNNNNN |
| 701 | E | 0.963 | 0.97560976 | 1488 | # EEEEEDEEEEEEEEEEEEEEEEEEEEEEEEEEEEEEEEEEE |
| 702 | Q | 0.937 | 0.95121951 | 1111 | # QQQQQKQQKQQQQQQQQQQQQQQQQQQQQQQQQQQQQQQQQ |
| 703 | V | 0.915 | 0.92682927 | 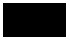 | # VVVVVVVVVVVVVIVVVVVVVVVVVVVVVIVVVVVVVVIVV |
| 704 | D | 1 | 1 | 2724 | # DDDDDDDDDDDDDDDDDDDDDDDDDDDDDDDDDDDDDDDDD |
| 705 | K | 0.926 | 0.95121951 | 973 | # KKKKKSKKKKKKKKKKKKKKKKKKKRKKKKKKKKKKKKKKK |
| 706 | L | 1 | 1 | 2725 | # LLLLLLLLLLLLLLLLLLLLLLLLLLLLLLLLLLLLLLLLL |
| 707 | V | 1 | 1 | 2726 | # VVVVVVVVVVVVVVVVVVVVVVVVVVVVVVVVVVVVVVVVV |
| 708 | S | 1 | 1 | 2727 | # SSSSSSSSSSSSSSSSSSSSSSSSSSSSSSSSSSSSSSSSS |
| 709 | A | 0.761 | 0.7804878 | 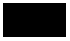 | # AAAASAAATAAAAAAAAAAASAAAATAAANAAAASASTATA |
| 710 | G | 0.963 | 0.97560976 | 1489 | # GGGGRGGGGGGGGGGGGGGGGGGGGGGGGGGGGGGGGGGGG |
| 711 | I | 0.937 | 0.95121951 | 1112 | # IIIIIIIIIIIIVIIIIIIIIIIIIIIIIIIIIVIIIIIII |
| 712 | R | 0.963 | 0.97560976 | 1490 | # RRRRKRRRRRRRRRRRRRRRRRRRRRRRRRRRRRRRRRRRR |
| 713 | K | 0.937 | 0.95121951 | 1113 | # KKKKKKKKKKKKKKKKKKKKKKKKKKKKKKKKKKKKKRKKR |
| 714 | V | 0.937 | 0.95121951 | 1114 | # VVVIVVVVVVVVVVVVVVVVIVVVVVVVVVVVVVVVVVVVV |
| 715 | L | 1 | 1 | 2728 | # LLLLLLLLLLLLLLLLLLLLLLLLLLLLLLLLLLLLLLLLL |

Table 2P

| | | | | | |
|---|---|---|---|---|---|
| 716 | F | 1 | 1 | 2729 | # FFFFFFFFFFFFFFFFFFFFFFFFFFFFFFFFFFFFFFFFF |
| 717 | L | 1 | 1 | 2730 | # LLLLLLLLLLLLLLLLLLLLLLLLLLLLLLLLLLLLLLLLL |
| 718 | D | 0.963 | 0.97560976 | 1491 | # DDDDDDDDDDDDDDDDDDDDDDDDDDNDDDDDDDDDDDDDD |
| 719 | G | 0.963 | 0.97560976 | 1492 | # GGGGRGGGGGGGGGGGGGGGGGGGGGGGGGGGGGGGGGGGG |
| 720 | I | 1 | 1 | 2731 | # IIIIIIIIIIIIIIIIIIIIIIIIIIIIIIIIIIIIIIIII |
| 721 | D | 0.963 | 0.97560976 | 1493 | # DDDDDDDDDDDDDDDDDDDDDDDDDDDDDDDDDDDDDEDDD |
| 722 | K | 0.937 | 0.95121951 | 1115 | # KKKKKKKKRKKKKKKKKKKKKKKRKKKKKKKKKKKKKKKKK |
| 723 | A | 1 | 1 | 2732 | # AAAAAAAAAAAAAAAAAAAAAAAAAAAAAAAAAAAAAAAAA |
| 724 | Q | 1 | 1 | 2733 | # QQQQQQQQQQQQQQQQQQQQQQQQQQQQQQQQQQQQQQQQQ |
| 725 | E | 0.84 | 0.80487805 | 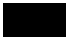 | # EEDEEDEEEEEEDEDEDDEEEEEEEDEEEEEEEEEEEEDEE |
| 726 | E | 0.769 | 0.68292683 | 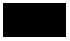 | # EEEDADEEDEEEEEEDEEEEDEEEEEEDEDDDEEDEEEDED |
| 727 | H | 1 | 1 | 2734 | # HHHHHHHHHHHHHHHHHHHHHHHHHHHHHHHHHHHHHHHHH |
| 728 | E | 1 | 1 | 2735 | # EEEEEEEEEEEEEEEEEEEEEEEEEEEEEEEEEEEEEEEEE |
| 729 | K | 0.926 | 0.95121951 | 974 | # KKKKKIKKKTKKKKKKKKKKKKKKKKKKKKKKKKKKKKKKK |
| 730 | Y | 1 | 1 | 2736 | # YYYYYYYYYYYYYYYYYYYYYYYYYYYYYYYYYYYYYYYYY |
| 731 | H | 1 | 1 | 2737 | # HHHHHHHHHHHHHHHHHHHHHHHHHHHHHHHHHHHHHHHHH |
| 732 | S | 0.89 | 0.92682927 | 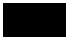 | # SSSSSSSNSSSSSCSSSSSSSSSSSSSSSSSSSSSTSSSSS |
| 733 | N | 1 | 1 | 2738 | # NNNNNNNNNNNNNNNNNNNNNNNNNNNNNNNNNNNNNNNNN |
| 734 | W | 0.963 | 0.97560976 | 1494 | # WWWW$WWWWWWWWWWWWWWWWWWWWWWWWWWWWWWWWWWWW |
| 735 | R | 0.963 | 0.97560976 | 1495 | # RRRRRRRRRRRRRRRRRRRRRRRRRRRRRRRKRRRRRRRRR |
| 736 | A | 1 | 1 | 2739 | # AAAAAAAAAAAAAAAAAAAAAAAAAAAAAAAAAAAAAAAAA |
| 737 | M | 0.937 | 0.95121951 | 1116 | # MMMMIMMMMMMMIMMMMMMMMMMMMMMMMMMMMMMMMMMMM |
| 738 | A | 1 | 1 | 2740 | # AAAAAAAAAAAAAAAAAAAAAAAAAAAAAAAAAAAAAAAAA |
| 739 | S | 0.793 | 0.82926829 | 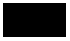 | # SSSDDNSSSSSSSSSNSSSSNGSSSSSSSSSSSSGSSSSSS |
| 740 | D | 0.937 | 0.95121951 | 1117 | # DDDDDDDDDDDDDDDDDDDDDDDDDDDDDDDDDDEDDEDDD |
| 741 | F | 1 | 1 | 2741 | # FFFFFFFFFFFFFFFFFFFFFFFFFFFFFFFFFFFFFFFFF |
| 742 | N | 0.963 | 0.97560976 | 1496 | # NNNNNYNNNNNNNNNNNNNNNNNNNNNNNNNNNNNNNNNNN |
| 743 | L | 0.963 | 0.97560976 | 1497 | # LLLLLLLILLLLLLLLLLLLLLLLLLLLLLLLLLLLLLLLL |
| 744 | P | 1 | 1 | 2742 | # PPPPPPPPPPPPPPPPPPPPPPPPPPPPPPPPPPPPPPPPP |
| 745 | P | 0.963 | 0.97560976 | 1498 | # PPPPPPPPPPPPPPPPPPPPPPPPPPPPPPPPPPPPPAPPP |
| 746 | V | 0.84 | 0.80487805 | 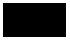 | # VVVIVIVVIVIVVVVVVVVVVVVVVVVIVIIVVVVVVVIVV |
| 747 | V | 1 | 1 | 2743 | # VVVVVVVVVVVVVVVVVVVVVVVVVVVVVVVVVVVVVVVVV |
| 748 | A | 1 | 1 | 2744 | # AAAAAAAAAAAAAAAAAAAAAAAAAAAAAAAAAAAAAAAAA |
| 749 | K | 1 | 1 | 2745 | # KKKKKKKKKKKKKKKKKKKKKKKKKKKKKKKKKKKKKKKKK |
| 750 | E | 0.901 | 0.92682927 | 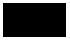 | # EEEEEDEEEEDEQEEEEEEEEEEEEEEEEEEEEEEEEEEEE |
| 751 | I | 1 | 1 | 2746 | # IIIIIIIIIIIIIIIIIIIIIIIIIIIIIIIIIIIIIIIII |
| 752 | V | 0.963 | 0.97560976 | 1499 | # VVVIVVVVVVVVVVVVVVVVVVVVVVVVVVVVVVVVVVVVV |
| 753 | A | 1 | 1 | 2747 | # AAAAAAAAAAAAAAAAAAAAAAAAAAAAAAAAAAAAAAAAA |
| 754 | S | 0.844 | 0.85365854 | 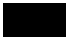 | # SSSCSSSCSSSSNSSSSSSSSCSSSSSSSSSSSSCSSCSSS |
| 755 | C | 1 | 1 | 2748 | # CCCCCCCCCCCCCCCCCCCCCCCCCCCCCCCCCCCCCCCCC |
| 756 | D | 0.963 | 0.97560976 | 1500 | # DDDDDDDDDDDDDDDDDDDDDDDDDDDDDDDDDDDNDDDDD |
| 757 | K | 1 | 1 | 2749 | # KKKKKKKKKKKKKKKKKKKKKKKKKKKKKKKKKKKKKKKKK |
| 758 | C | 1 | 1 | 2750 | # CCCCCCCCCCCCCCCCCCCCCCCCCCCCCCCCCCCCCCCCC |
| 759 | Q | 1 | 1 | 2751 | # QQQQQQQQQQQQQQQQQQQQQQQQQQQQQQQQQQQQQQQQQ |
| 760 | L | 0.879 | 0.90243902 | 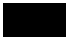 | # LLLLVQLLLLLLVLLLLLLLLLLLLLLLLLLLLLLLLVLLL |
| 761 | K | 1 | 1 | 2752 | # KKKKKKKKKKKKKKKKKKKKKKKKKKKKKKKKKKKKKKKKK |
| 762 | G | 1 | 1 | 2753 | # GGGGGGGGGGGGGGGGGGGGGGGGGGGGGGGGGGGGGGGGG |
| 763 | E | 1 | 1 | 2754 | # EEEEEEEEEEEEEEEEEEEEEEEEEEEEEEEEEEEEEEEEE |

Table 2Q

| | | | | | |
|---|---|---|---|---|---|
| 764 | A | 1 | 1 | 2755 | # AAAAAAAAAAAAAAAAAAAAAAAAAAAAAAAAAAAAAAAAA |
| 765 | M | 0.926 | 0.95121951 | 975 | # MMMIMMMMMTMMMMMMMMMMMMMMMMMMMMMMMMMMMMMMM |
| 766 | H | 1 | 1 | 2756 | # HHHHHHHHHHHHHHHHHHHHHHHHHHHHHHHHHHHHHHHHH |
| 767 | G | 1 | 1 | 2757 | # GGGGGGGGGGGGGGGGGGGGGGGGGGGGGGGGGGGGGGGGG |
| 768 | Q | 1 | 1 | 2758 | # QQQQQQQQQQQQQQQQQQQQQQQQQQQQQQQQQQQQQQQQQ |
| 769 | V | 1 | 1 | 2759 | # VVVVVVVVVVVVVVVVVVVVVVVVVVVVVVVVVVVVVVVVV |
| 770 | D | 1 | 1 | 2760 | # DDDDDDDDDDDDDDDDDDDDDDDDDDDDDDDDDDDDDDDDD |
| 771 | C | 0.963 | 0.97560976 | 1501 | # CCCCCCCCCCFCCCCCCCCCCCCCCCCCCCCCCCCCCCCCC |
| 772 | S | 1 | 1 | 2761 | # SSSSSSSSSSSSSSSSSSSSSSSSSSSSSSSSSSSSSSSSS |
| 773 | P | 1 | 1 | 2762 | # PPPPPPPPPPPPPPPPPPPPPPPPPPPPPPPPPPPPPPPPP |
| 774 | G | 1 | 1 | 2763 | # GGGGGGGGGGGGGGGGGGGGGGGGGGGGGGGGGGGGGGGGG |
| 775 | I | 0.963 | 0.97560976 | 1502 | # IIIIILIIIIIIIIIIIIIIIIIIIIIIIIIIIIIIIIIII |
| 776 | W | 1 | 1 | 2764 | # WWWWWWWWWWWWWWWWWWWWWWWWWWWWWWWWWWWWWWWWW |
| 777 | Q | 1 | 1 | 2765 | # QQQQQQQQQQQQQQQQQQQQQQQQQQQQQQQQQQQQQQQQQ |
| 778 | L | 1 | 1 | 2766 | # LLLLLLLLLLLLLLLLLLLLLLLLLLLLLLLLLLLLLLLLL |
| 779 | D | 1 | 1 | 2767 | # DDDDDDDDDDDDDDDDDDDDDDDDDDDDDDDDDDDDDDDDD |
| 780 | C | 1 | 1 | 2768 | # CCCCCCCCCCCCCCCCCCCCCCCCCCCCCCCCCCCCCCCCC |
| 781 | T | 1 | 1 | 2769 | # TTTTTTTTTTTTTTTTTTTTTTTTTTTTTTTTTTTTTTTTT |
| 782 | H | 1 | 1 | 2770 | # HHHHHHHHHHHHHHHHHHHHHHHHHHHHHHHHHHHHHHHHH |
| 783 | L | 1 | 1 | 2771 | # LLLLLLLLLLLLLLLLLLLLLLLLLLLLLLLLLLLLLLLLL |
| 784 | E | 0.963 | 0.97560976 | 1503 | # EEEEEEEEEEDEEEEEEEEEEEEEEEEEEEEEEEEEEEEEE |
| 785 | G | 0.963 | 0.97560976 | 1504 | # GGGGGGGGGGGEGGGGGGGGGGGGGGGGGGGGGGGGGGGGG |
| 786 | K | 0.963 | 0.97560976 | 1505 | # KKKKKKKKKKNKKKKKKKKKKKKKKKKKKKKKKKKKKKKKK |
| 787 | I | 0.774 | 0.53658537 | 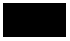 | # VIVVIVVVIVIVIIVIVVIVIVIIVIIVVIIIVIIVIVIII |
| 788 | I | 1 | 1 | 2772 | # IIIIIIIIIIIIIIIIIIIIIIIIIIIIIIIIIIIIIIIII |
| 789 | L | 1 | 1 | 2773 | # LLLLLLLLLLLLLLLLLLLLLLLLLLLLLLLLLLLLLLLLL |
| 790 | V | 1 | 1 | 2774 | # VVVVVVVVVVVVVVVVVVVVVVVVVVVVVVVVVVVVVVVVV |
| 791 | A | 1 | 1 | 2775 | # AAAAAAAAAAAAAAAAAAAAAAAAAAAAAAAAAAAAAAAAA |
| 792 | V | 1 | 1 | 2776 | # VVVVVVVVVVVVVVVVVVVVVVVVVVVVVVVVVVVVVVVVV |
| 793 | H | 1 | 1 | 2777 | # HHHHHHHHHHHHHHHHHHHHHHHHHHHHHHHHHHHHHHHHH |
| 794 | V | 1 | 1 | 2778 | # VVVVVVVVVVVVVVVVVVVVVVVVVVVVVVVVVVVVVVVVV |
| 795 | A | 1 | 1 | 2779 | # AAAAAAAAAAAAAAAAAAAAAAAAAAAAAAAAAAAAAAAAA |
| 796 | S | 1 | 1 | 2780 | # SSSSSSSSSSSSSSSSSSSSSSSSSSSSSSSSSSSSSSSSS |
| 797 | G | 0.963 | 0.97560976 | 1506 | # GGGGRGGGGGGGGGGGGGGGGGGGGGGGGGGGGGGGGGGGG |
| 798 | Y | 1 | 1 | 2781 | # YYYYYYYYYYYYYYYYYYYYYYYYYYYYYYYYYYYYYYYYY |
| 799 | I | 0.963 | 0.97560976 | 1507 | # IIILIIIIIIIIIIIIIIIIIIIIIIIIIIIIIIIIIIIII |
| 800 | E | 0.963 | 0.97560976 | 1508 | # EEEEEEEEEEDEEEEEEEEEEEEEEEEEEEEEEEEEEEEEE |
| 801 | A | 1 | 1 | 2782 | # AAAAAAAAAAAAAAAAAAAAAAAAAAAAAAAAAAAAAAAAA |
| 802 | E | 1 | 1 | 2783 | # EEEEEEEEEEEEEEEEEEEEEEEEEEEEEEEEEEEEEEEEE |
| 803 | V | 1 | 1 | 2784 | # VVVVVVVVVVVVVVVVVVVVVVVVVVVVVVVVVVVVVVVVV |
| 804 | I | 1 | 1 | 2785 | # IIIIIIIIIIIIIIIIIIIIIIIIIIIIIIIIIIIIIIIII |
| 805 | P | 1 | 1 | 2786 | # PPPPPPPPPPPPPPPPPPPPPPPPPPPPPPPPPPPPPPPPP |
| 806 | A | 0.963 | 0.97560976 | 1509 | # AAAAAAAAAAAAAAAVAAAAAAAAAAAAAAAAAAAAAAAAA |
| 807 | E | 0.963 | 0.97560976 | 1510 | # EEEEEEEEEEEDEEEEEEEEEEEEEEEEEEEEEEEEEEEEE |
| 808 | T | 1 | 1 | 2787 | # TTTTTTTTTTTTTTTTTTTTTTTTTTTTTTTTTTTTTTTTT |
| 809 | G | 0.963 | 0.97560976 | 1511 | # GGGGGGGGGGGAGGGGGGGGGGGGGGGGGGGGGGGGGGGGG |
| 810 | Q | 1 | 1 | 2788 | # QQQQQQQQQQQQQQQQQQQQQQQQQQQQQQQQQQQQQQQQQ |
| 811 | E | 0.897 | 0.90243902 | 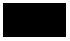 | # EEEEEEEEEDDDEEEEEEEEDEEEEEEEEEEEEEEEEEEEE |

Table 2R

| | | | | | |
|---|---|---|---|---|---|
| 812 | T | 1 | 1 | 2789 | # TTTTTTTTTTTTTTTTTTTTTTTTTTTTTTTTTTTTTTTTT |
| 813 | A | 1 | 1 | 2790 | # AAAAAAAAAAAAAAAAAAAAAAAAAAAAAAAAAAAAAAAAA |
| 814 | Y | 1 | 1 | 2791 | # YYYYYYYYYYYYYYYYYYYYYYYYYYYYYYYYYYYYYYYYY |
| 815 | F | 1 | 1 | 2792 | # FFFFFFFFFFFFFFFFFFFFFFFFFFFFFFFFFFFFFFFFF |
| 816 | L | 0.786 | 0.63414634 | 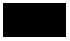 | # LILLLLLLILLLILILLLILILILLILLLILLLLILIIIII |
| 817 | L | 1 | 1 | 2793 | # LLLLLLLLLLLLLLLLLLLLLLLLLLLLLLLLLLLLLLLLL |
| 818 | K | 0.926 | 0.95121951 | 976 | # KKKKKKKKKKTRKKKKKKKKKKKKKKKKKKKKKKKKKKKKK |
| 819 | L | 1 | 1 | 2794 | # LLLLLLLLLLLLLLLLLLLLLLLLLLLLLLLLLLLLLLLLL |
| 820 | A | 1 | 1 | 2795 | # AAAAAAAAAAAAAAAAAAAAAAAAAAAAAAAAAAAAAAAAA |
| 821 | G | 1 | 1 | 2796 | # GGGGGGGGGGGGGGGGGGGGGGGGGGGGGGGGGGGGGGGGG |
| 822 | R | 1 | 1 | 2797 | # RRRRRRRRRRRRRRRRRRRRRRRRRRRRRRRRRRRRRRRRR |
| 823 | W | 1 | 1 | 2798 | # WWWWWWWWWWWWWWWWWWWWWWWWWWWWWWWWWWWWWWWWW |
| 824 | P | 1 | 1 | 2799 | # PPPPPPPPPPPPPPPPPPPPPPPPPPPPPPPPPPPPPPPPP |
| 825 | V | 1 | 1 | 2800 | # VVVVVVVVVVVVVVVVVVVVVVVVVVVVVVVVVVVVVVVVV |
| 826 | K | 0.83 | 0.82926829 | 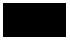 | # TKKKKKKKTKNKKKKKKKKKKKTKKKKKKTTKTKKKKKKKK |
| 827 | T | 0.926 | 0.95121951 | 977 | # TTTTTTATTTTTTTTTTTTTTTTTTVTTTTTTTTTTTTTTT |
| 828 | I | 0.844 | 0.85365854 | 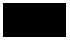 | # IIIIIVIIIILIIIIVIIIIIIIIVIIIVIIIIIIIIIIVI |
| 829 | H | 1 | 1 | 2801 | # HHHHHHHHHHHHHHHHHHHHHHHHHHHHHHHHHHHHHHHHH |
| 830 | T | 1 | 1 | 2802 | # TTTTTTTTTTTTTTTTTTTTTTTTTTTTTTTTTTTTTTTTT |
| 831 | D | 1 | 1 | 2803 | # DDDDDDDDDDDDDDDDDDDDDDDDDDDDDDDDDDDDDDDDD |
| 832 | N | 1 | 1 | 2804 | # NNNNNNNNNNNNNNNNNNNNNNNNNNNNNNNNNNNNNNNNN |
| 833 | G | 1 | 1 | 2805 | # GGGGGGGGGGGGGGGGGGGGGGGGGGGGGGGGGGGGGGGGG |
| 834 | S | 0.711 | 0.70731707 | 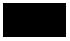 | # SPSPPPSPSSSSSRSPSSSGRSSSSSSPSTSSSSSSSSPSS |
| 835 | N | 0.963 | 0.97560976 | 1512 | # NNNNNINNNNNNNNNNNNNNNNNNNNNNNNNNNNNNNNNNN |
| 836 | F | 1 | 1 | 2806 | # FFFFFFFFFFFFFFFFFFFFFFFFFFFFFFFFFFFFFFFFF |
| 837 | T | 0.816 | 0.80487805 | 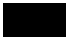 | # TTTVTITTIITITTTTTTTITTTTTTTTTTTTTTITTTITT |
| 838 | S | 0.937 | 0.95121951 | 1118 | # SSSSSSSSSSSSSSSSGGSSSSSSSSSSSSSSSSSSSSSSS |
| 839 | T | 0.744 | 0.70731707 | 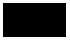 | # TTATTTTNTTTANNTTAATTTNAATTTTTTTTATATTTTTT |
| 840 | T | 0.83 | 0.82926829 | 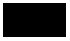 | # TTTAAATTTTTTASTTTTTTTTTATTTTTTTTTTTTTTATT |
| 841 | V | 1 | 1 | 2807 | # VVVVVVVVVVVVVVVVVVVVVVVVVVVVVVVVVVVVVVVVV |
| 842 | K | 0.937 | 0.95121951 | 1119 | # KKKKKKKKKKKKKKKKRRKKKKKKKKKKKKKKKKKKKKKKK |
| 843 | A | 1 | 1 | 2808 | # AAAAAAAAAAAAAAAAAAAAAAAAAAAAAAAAAAAAAAAAA |
| 844 | A | 1 | 1 | 2809 | # AAAAAAAAAAAAAAAAAAAAAAAAAAAAAAAAAAAAAAAAA |
| 845 | C | 1 | 1 | 2810 | # CCCCCCCCCCCCCCCCCCCCCCCCCCCCCCCCCCCCCCCCC |
| 846 | W | 0.963 | 0.97560976 | 1513 | # WWWW$WWWWWWWWWWWWWWWWWWWWWWWWWWWWWWWWWWWW |
| 847 | W | 0.937 | 0.95121951 | 1120 | # WWWW$WWWWWWWWWWWWWWWW$WWWWWWWWWWWWWWWWWWW |
| 848 | A | 0.963 | 0.97560976 | 1514 | # AAAAAAAAAAAAAAAAAAAAAAAAAAATAAAAAAAAAAAAA |
| 849 | G | 0.926 | 0.95121951 | 978 | # GGGGEGGGGGGGGGGGGGGGGRGGGGGGGGGGGGGGGGGGG |
| 850 | I | 0.937 | 0.95121951 | 1121 | # IIIIIIIIIIIIVIVIIIIIIIIIIIIIIIIIIIIIIIIII |
| 851 | K | 0.963 | 0.97560976 | 1515 | # KKKKKKKKKKTKKKKKKKKKKKKKKKKKKKKKKKKKKKKKK |
| 852 | Q | 1 | 1 | 2811 | # QQQQQQQQQQQQQQQQQQQQQQQQQQQQQQQQQQQQQQQQQ |
| 853 | E | 0.963 | 0.97560976 | 1516 | # EEEEEEEEEEEEEEEEEEEEEEEKEEEEEEEEEEEEEEEEE |
| 854 | F | 1 | 1 | 2812 | # FFFFFFFFFFFFFFFFFFFFFFFFFFFFFFFFFFFFFFFFF |
| 855 | G | 1 | 1 | 2813 | # GGGGGGGGGGGGGGGGGGGGGGGGGGGGGGGGGGGGGGGGG |
| 856 | I | 0.963 | 0.97560976 | 1517 | # IIIIIIIITIIIIIIIIIIIIIIIIIIIIIIIIIIIIIIII |
| 857 | P | 1 | 1 | 2814 | # PPPPPPPPPPPPPPPPPPPPPPPPPPPPPPPPPPPPPPPPP |
| 858 | Y | 1 | 1 | 2815 | # YYYYYYYYYYYYYYYYYYYYYYYYYYYYYYYYYYYYYYYYY |
| 859 | N | 1 | 1 | 2816 | # NNNNNNNNNNNNNNNNNNNNNNNNNNNNNNNNNNNNNNNNN |

Table 2S

| | | | | | |
|---|---|---|---|---|---|
| 860 | P | 1 | 1 | 2817 | # PPPPPPPPPPPPPPPPPPPPPPPPPPPPPPPPPPPPPPPPP |
| 861 | Q | 1 | 1 | 2818 | # QQQQQQQQQQQQQQQQQQQQQQQQQQQQQQQQQQQQQQQQQ |
| 862 | S | 0.963 | 0.97560976 | 1518 | # SSSSSRSSSSSSSSSSSSSSSSSSSSSSSSSSSSSSSSSSS |
| 863 | Q | 1 | 1 | 2819 | # QQQQQQQQQQQQQQQQQQQQQQQQQQQQQQQQQQQQQQQQQ |
| 864 | G | 1 | 1 | 2820 | # GGGGGGGGGGGGGGGGGGGGGGGGGGGGGGGGGGGGGGGGG |
| 865 | V | 1 | 1 | 2821 | # VVVVVVVVVVVVVVVVVVVVVVVVVVVVVVVVVVVVVVVVV |
| 866 | V | 0.865 | 0.85365854 | 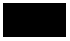 | # VIVVVVVVVVVVVVVVVVVVVVVIIVVIIVVVVVVVIVVVV |
| 867 | E | 0.926 | 0.95121951 | 979 | # EEEEEDEEEEAEEEEEEEEEEEEEEEEEEEEEEEEEEEEEE |
| 868 | S | 1 | 1 | 2822 | # SSSSSSSSSSSSSSSSSSSSSSSSSSSSSSSSSSSSSSSSS |
| 869 | M | 0.963 | 0.97560976 | 1519 | # MMMMMMMIMMMMMMMMMMMMMMMMMMMMMMMMMMMMMMMMM |
| 870 | N | 0.926 | 0.95121951 | 980 | # NNNNNNNNNNHNNDNNNNNNNNNNNNNNNNNNNNNNNNNNN |
| 871 | K | 0.805 | 0.80487805 | 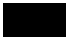 | # KKKKKNKKKKRRNKKKKKNNKKKKKKNKKKKKKKKKKKNKK |
| 872 | E | 0.89 | 0.92682927 | 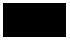 | # EEEEEDEEEEAEEEEEEEEEEEEEEQEEEEEEEEEEEEEEE |
| 873 | L | 1 | 1 | 2823 | # LLLLLLLLLLLLLLLLLLLLLLLLLLLLLLLLLLLLLLLLL |
| 874 | K | 0.963 | 0.97560976 | 1520 | # KKKKKTKKKKKKKKKKKKKKKKKKKKKKKKKKKKKKKKKKK |
| 875 | K | 0.926 | 0.95121951 | 981 | # KKKKKTKKKKKKKKKKKKKKKKKKKQKKKKKKKKKKKKKKK |
| 876 | I | 1 | 1 | 2824 | # IIIIIIIIIIIIIIIIIIIIIIIIIIIIIIIIIIIIIIIII |
| 877 | I | 1 | 1 | 2825 | # IIIIIIIIIIIIIIIIIIIIIIIIIIIIIIIIIIIIIIIII |
| 878 | G | 0.963 | 0.97560976 | 1521 | # GGGGGGGGGGGGGGGGGGGGGGGGGGGGGGGGGGGGGEGGG |
| 879 | Q | 1 | 1 | 2826 | # QQQQQQQQQQQQQQQQQQQQQQQQQQQQQQQQQQQQQQQQQ |
| 880 | V | 1 | 1 | 2827 | # VVVVVVVVVVVVVVVVVVVVVVVVVVVVVVVVVVVVVVVVV |
| 881 | R | 1 | 1 | 2828 | # RRRRRRRRRRRRRRRRRRRRRRRRRRRRRRRRRRRRRRRRR |
| 882 | D | 1 | 1 | 2829 | # DDDDDDDDDDDDDDDDDDDDDDDDDDDDDDDDDDDDDDDDD |
| 883 | Q | 1 | 1 | 2830 | # QQQQQQQQQQQQQQQQQQQQQQQQQQQQQQQQQQQQQQQQQ |
| 884 | A | 1 | 1 | 2831 | # AAAAAAAAAAAAAAAAAAAAAAAAAAAAAAAAAAAAAAAAA |
| 885 | E | 1 | 1 | 2832 | # EEEEEEEEEEEEEEEEEEEEEEEEEEEEEEEEEEEEEEEEE |
| 886 | H | 1 | 1 | 2833 | # HHHHHHHHHHHHHHHHHHHHHHHHHHHHHHHHHHHHHHHHH |
| 887 | L | 1 | 1 | 2834 | # LLLLLLLLLLLLLLLLLLLLLLLLLLLLLLLLLLLLLLLLL |
| 888 | K | 1 | 1 | 2835 | # KKKKKKKKKKKKKKKKKKKKKKKKKKKKKKKKKKKKKKKKK |
| 889 | T | 0.963 | 0.97560976 | 1522 | # TTTTTTTTTTTTTTTTTTTTTTTTTTTRTTTTTTTTTTTTT |
| 890 | A | 1 | 1 | 2836 | # AAAAAAAAAAAAAAAAAAAAAAAAAAAAAAAAAAAAAAAAA |
| 891 | V | 1 | 1 | 2837 | # VVVVVVVVVVVVVVVVVVVVVVVVVVVVVVVVVVVVVVVVV |
| 892 | Q | 0.963 | 0.97560976 | 1523 | # QQQQQQQQQQQQQQQQQQQQQQQQHQQQQQQQQQQQQQQQQ |
| 893 | M | 1 | 1 | 2838 | # MMMMMMMMMMMMMMMMMMMMMMMMMMMMMMMMMMMMMMMMM |
| 894 | A | 1 | 1 | 2839 | # AAAAAAAAAAAAAAAAAAAAAAAAAAAAAAAAAAAAAAAAA |
| 895 | V | 1 | 1 | 2840 | # VVVVVVVVVVVVVVVVVVVVVVVVVVVVVVVVVVVVVVVVV |
| 896 | F | 0.963 | 0.97560976 | 1524 | # FFFFFFFFFFFFFFFFFFFFFFFFFFFFFFFFFFFFFFFLF |
| 897 | I | 1 | 1 | 2841 | # IIIIIIIIIIIIIIIIIIIIIIIIIIIIIIIIIIIIIIIII |
| 898 | H | 1 | 1 | 2842 | # HHHHHHHHHHHHHHHHHHHHHHHHHHHHHHHHHHHHHHHHH |
| 899 | N | 1 | 1 | 2843 | # NNNNNNNNNNNNNNNNNNNNNNNNNNNNNNNNNNNNNNNNN |
| 900 | F | 0.963 | 0.97560976 | 1525 | # FFFFFFFFFYFFFFFFFFFFFFFFFFFFFFFFFFFFFFFFF |
| 901 | K | 0.963 | 0.97560976 | 1526 | # KKKKKKKKKKIKKKKKKKKKKKKKKKKKKKKKKKKKKKKKK |
| 902 | R | 1 | 1 | 2844 | # RRRRRRRRRRRRRRRRRRRRRRRRRRRRRRRRRRRRRRRRR |
| 903 | K | 1 | 1 | 2845 | # KKKKKKKKKKKKKKKKKKKKKKKKKKKKKKKKKKKKKKKKK |
| 904 | G | 1 | 1 | 2846 | # GGGGGGGGGGGGGGGGGGGGGGGGGGGGGGGGGGGGGGGGG |
| 905 | G | 1 | 1 | 2847 | # GGGGGGGGGGGGGGGGGGGGGGGGGGGGGGGGGGGGGGGGG |
| 906 | I | 1 | 1 | 2848 | # IIIIIIIIIIIIIIIIIIIIIIIIIIIIIIIIIIIIIIIII |
| 907 | G | 1 | 1 | 2849 | # GGGGGGGGGGGGGGGGGGGGGGGGGGGGGGGGGGGGGGGGG |

Table 2T

| | | | | | |
|---|---|---|---|---|---|
| 908 | G | 0.963 | 0.97560976 | 1527 | # GGGGGGDGGGGGGGGGGGGGGGGGGGGGGGGGGGGGGGGGG |
| 909 | Y | 1 | 1 | 2850 | # YYYYYYYYYYYYYYYYYYYYYYYYYYYYYYYYYYYYYYYYY |
| 910 | S | 0.963 | 0.97560976 | 1528 | # SSSSSSCSSSSSSSSSSSSSSSSSSSSSSSSSSSSSSSSSS |
| 911 | A | 1 | 1 | 2851 | # AAAAAAAAAAAAAAAAAAAAAAAAAAAAAAAAAAAAAAAAA |
| 912 | G | 1 | 1 | 2852 | # GGGGGGGGGGGGGGGGGGGGGGGGGGGGGGGGGGGGGGGGG |
| 913 | E | 0.963 | 0.97560976 | 1529 | # GEEEEEEEEEEEEEEEEEEEEEEEEEEEEEEEEEEEEEEEE |
| 914 | R | 1 | 1 | 2853 | # RRRRRRRRRRRRRRRRRRRRRRRRRRRRRRRRRRRRRRRRR |
| 915 | I | 1 | 1 | 2854 | # IIIIIIIIIIIIIIIIIIIIIIIIIIIIIIIIIIIIIIIII |
| 916 | V | 0.82 | 0.75609756 | 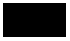 | # VIVIVIVIVVVVVVVVVVVIVIVVVVVVVVIVVVIVIVVVI |
| 917 | D | 0.963 | 0.97560976 | 1530 | # DDDDDDDDDDDDDDDDDDDDDDDDDDDGDDDDDDDDDDDDD |
| 918 | I | 0.963 | 0.97560976 | 1531 | # IIIIIIIIIIIIIIIIIIIIIIIIIIIIIMIIIIIIIIIII |
| 919 | I | 1 | 1 | 2855 | # IIIIIIIIIIIIIIIIIIIIIIIIIIIIIIIIIIIIIIIII |
| 920 | A | 1 | 1 | 2856 | # AAAAAAAAAAAAAAAAAAAAAAAAAAAAAAAAAAAAAAAAA |
| 921 | T | 0.897 | 0.90243902 | 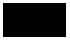 | # TTTTTSTTSTTTTTTTTTTTTTTTTTTTTTTTTTTSTSTTT |
| 922 | D | 1 | 1 | 2857 | # DDDDDDDDDDDDDDDDDDDDDDDDDDDDDDDDDDDDDDDDD |
| 923 | I | 0.926 | 0.95121951 | 982 | # IIIIIIIIIMIIIIIIIIIIIIIIIIIIIIIIIIIIIIIII |
| 924 | Q | 1 | 1 | 2858 | # QQQQQQQQQQQQQQQQQQQQQQQQQQQQQQQQQQQQQQQQQ |
| 925 | T | 0.963 | 0.97560976 | 1532 | # TTTTTTTTTTTTTTTTTTTTTTTTTTTTITTTTTTTTTTTT |
| 926 | K | 0.926 | 0.95121951 | 983 | # KKKKKKKKKKKKKRKKKKKKKKKKKKKKKKKKKKKKQKKKK |
| 927 | E | 0.963 | 0.97560976 | 1533 | # EEEEEEEEEEEEEEEEEEEEEEEEEEEEEEEEEEEEQEEEE |
| 928 | L | 1 | 1 | 2859 | # LLLLLLLLLLLLLLLLLLLLLLLLLLLLLLLLLLLLLLLLL |
| 929 | Q | 1 | 1 | 2860 | # QQQQQQQQQQQQQQQQQQQQQQQQQQQQQQQQQQQQQQQQQ |
| 930 | K | 0.937 | 0.95121951 | 1122 | # KNKKKKKKKKKKKKKKKKKKKKKKKKKKKKKKKKKKKKKKN |
| 931 | Q | 1 | 1 | 2861 | # QQQQQQQQQQQQQQQQQQQQQQQQQQQQQQQQQQQQQQQQQ |
| 932 | I | 0.963 | 0.97560976 | 1534 | # IIIIIIIIIIIIIIIIIIIIIVIIIIIIIIIIIIIIIIIII |
| 933 | T | 0.963 | 0.97560976 | 1535 | # TTTTTTTTTTTTTTTTTTTTTTTTTTTTTTTTTTTTTTTTI |
| 934 | K | 0.963 | 0.97560976 | 1536 | # KKKKKKKKKRKKKKKKKKKKKKKKKKKKKKKKKKKKKKKKK |
| 935 | I | 0.963 | 0.97560976 | 1537 | # IIIIIVIIIIIIIIIIIIIIIIIIIIIIIIIIIIIIIIIII |
| 936 | Q | 1 | 1 | 2862 | # QQQQQQQQQQQQQQQQQQQQQQQQQQQQQQQQQQQQQQQQQ |
| 937 | N | 0.963 | 0.97560976 | 1538 | # NNNNNNNNNHNNNNNNNNNNNNNNNNNNNNNNNNNNNNNNN |
| 938 | F | 1 | 1 | 2863 | # FFFFFFFFFFFFFFFFFFFFFFFFFFFFFFFFFFFFFFFFF |
| 939 | R | 1 | 1 | 2864 | # RRRRRRRRRRRRRRRRRRRRRRRRRRRRRRRRRRRRRRRRR |
| 940 | V | 1 | 1 | 2865 | # VVVVVVVVVVVVVVVVVVVVVVVVVVVVVVVVVVVVVVVVV |
| 941 | Y | 1 | 1 | 2866 | # YYYYYYYYYYYYYYYYYYYYYYYYYYYYYYYYYYYYYYYYY |
| 942 | Y | 1 | 1 | 2867 | # YYYYYYYYYYYYYYYYYYYYYYYYYYYYYYYYYYYYYYYYY |
| 943 | R | 1 | 1 | 2868 | # RRRRRRRRRRRRRRRRRRRRRRRRRRRRRRRRRRRRRRRRR |
| 944 | D | 0.963 | 0.97560976 | 1539 | # DDDDDDDDDDDDDGDDDDDDDDDDDDDDDDDDDDDDDDDDD |
| 945 | S | 0.865 | 0.85365854 | 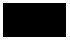 | # SSSSNSSSNSSSSSSSSSSSSSSNSSNSSSNSSNSSSSSSS |
| 946 | R | 0.937 | 0.95121951 | 1123 | # RRRRRRRRRRRRRRRRRRRRRRRRRRKRRRRRRKRRRRRRR |
| 947 | D | 0.833 | 0.85365854 | 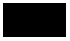 | # NDDDDDDDDDDDDDDENNEDDDDDDDDDDDDEDDDDDDDDD |
| 948 | P | 0.963 | 0.97560976 | 1540 | # PPPPPPPPPPPPPPPPPSPPPPPPPPPPPPPPPPPPPPPPP |
| 949 | L | 0.839 | 0.87804878 | 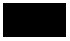 | # LLLILLLLLLLLLLLFLLLLLLLLVLLLVLILLLLLLLLLL |
| 950 | W | 1 | 1 | 2869 | # WWWWWWWWWWWWWWWWWWWWWWWWWWWWWWWWWWWWWWWWW |
| 951 | K | 1 | 1 | 2870 | # KKKKKKKKKKKKKKKKKKKKKKKKKKKKKKKKKKKKKKKKK |
| 952 | G | 1 | 1 | 2871 | # GGGGGGGGGGGGGGGGGGGGGGGGGGGGGGGGGGGGGGGGG |
| 953 | P | 0.963 | 0.97560976 | 1541 | # PPPPPPPPPPPPPPPPPPPPPPPPPHPPPPPPPPPPPPPPP |
| 954 | A | 1 | 1 | 2872 | # AAAAAAAAAAAAAAAAAAAAAAAAAAAAAAAAAAAAAAAAA |
| 955 | K | 1 | 1 | 2873 | # KKKKKKKKKKKKKKKKKKKKKKKKKKKKKKKKKKKKKKKKK |

Table 2U

| | | | | | |
|---|---|---|---|---|---|
| 956 | L | 1 | 1 | 2874 | # LLLLLLLLLLLLLLLLLLLLLLLLLLLLLLLLLLLLLLLLL |
| 957 | L | 1 | 1 | 2875 | # LLLLLLLLLLLLLLLLLLLLLLLLLLLLLLLLLLLLLLLLL |
| 958 | W | 0.963 | 0.97560976 | 1542 | # WWWW$WWWWWWWWWWWWWWWWWWWWWWWWWWWWWWWWWWWW |
| 959 | K | 1 | 1 | 2876 | # KKKKKKKKKKKKKKKKKKKKKKKKKKKKKKKKKKKKKKKKK |
| 960 | G | 1 | 1 | 2877 | # GGGGGGGGGGGGGGGGGGGGGGGGGGGGGGGGGGGGGGGGG |
| 961 | E | 1 | 1 | 2878 | # EEEEEEEEEEEEEEEEEEEEEEEEEEEEEEEEEEEEEEEEE |
| 962 | G | 1 | 1 | 2879 | # GGGGGGGGGGGGGGGGGGGGGGGGGGGGGGGGGGGGGGGGG |
| 963 | A | 1 | 1 | 2880 | # AAAAAAAAAAAAAAAAAAAAAAAAAAAAAAAAAAAAAAAAA |
| 964 | V | 1 | 1 | 2881 | # VVVVVVVVVVVVVVVVVVVVVVVVVVVVVVVVVVVVVVVVV |
| 965 | V | 1 | 1 | 2882 | # VVVVVVVVVVVVVVVVVVVVVVVVVVVVVVVVVVVVVVVVV |
| 966 | I | 1 | 1 | 2883 | # IIIIIIIIIIIIIIIIIIIIIIIIIIIIIIIIIIIIIIIII |
| 967 | Q | 1 | 1 | 2884 | # QQQQQQQQQQQQQQQQQQQQQQQQQQQQQQQQQQQQQQQQQ |
| 968 | D | 0.963 | 0.97560976 | 1543 | # DDDEDDDDDDDDDDDDDDDDDDDDDDDDDDDDDDDDDDDDD |
| 969 | N | 0.963 | 0.97560976 | 1544 | # NNNNNNNNNNNNNNNNNNNNNNNNNNNNNNNNNNNNNKNNN |
| 970 | S | 0.963 | 0.97560976 | 1545 | # SSSSSSSSSSSSSSSSSSSSSSSSSSSNSSSSSSSSSSSSS |
| 971 | D | 0.88 | 0.87804878 | 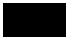 | # EEDDDDDDDDDDDDEDDDDDDDDDDDDDDDDDDDDDDDEDE |
| 972 | I | 1 | 1 | 2885 | # IIIIIIIIIIIIIIIIIIIIIIIIIIIIIIIIIIIIIIIII |
| 973 | K | 1 | 1 | 2886 | # KKKKKKKKKKKKKKKKKKKKKKKKKKKKKKKKKKKKKKKKK |
| 974 | V | 1 | 1 | 2887 | # VVVVVVVVVVVVVVVVVVVVVVVVVVVVVVVVVVVVVVVVV |
| 975 | V | 1 | 1 | 2888 | # VVVVVVVVVVVVVVVVVVVVVVVVVVVVVVVVVVVVVVVVV |
| 976 | P | 1 | 1 | 2889 | # PPPPPPPPPPPPPPPPPPPPPPPPPPPPPPPPPPPPPPPPP |
| 977 | R | 1 | 1 | 2890 | # RRRRRRRRRRRRRRRRRRRRRRRRRRRRRRRRRRRRRRRRR |
| 978 | R | 1 | 1 | 2891 | # RRRRRRRRRRRRRRRRRRRRRRRRRRRRRRRRRRRRRRRRR |
| 979 | K | 1 | 1 | 2892 | # KKKKKKKKKKKKKKKKKKKKKKKKKKKKKKKKKKKKKKKKK |
| 980 | A | 0.829 | 0.7804878 | 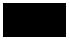 | # VAAAAAAAVVVVAVAAAAAAAAAAAAAAAAVVAAAAAAAVA |
| 981 | K | 1 | 1 | 2893 | # KKKKKKKKKKKKKKKKKKKKKKKKKKKKKKKKKKKKKKKKK |
| 982 | I | 0.963 | 0.97560976 | 1546 | # IIIIIIIIIIIIIIIIIIIIIIIIIIIVIIIIIIIIIIIII |
| 983 | I | 1 | 1 | 2894 | # IIIIIIIIIIIIIIIIIIIIIIIIIIIIIIIIIIIIIIIII |
| 984 | R | 1 | 1 | 2895 | # RRRRRRRRRRRRRRRRRRRRRRRRRRRRRRRRRRRRRRRRR |
| 985 | D | 1 | 1 | 2896 | # DDDDDDDDDDDDDDDDDDDDDDDDDDDDDDDDDDDDDDDDD |
| 986 | Y | 1 | 1 | 2897 | # YYYYYYYYYYYYYYYYYYYYYYYYYYYYYYYYYYYYYYYYY |
| 987 | G | 1 | 1 | 2898 | # GGGGGGGGGGGGGGGGGGGGGGGGGGGGGGGGGGGGGGGGG |
| 988 | K | 1 | 1 | 2899 | # KKKKKKKKKKKKKKKKKKKKKKKKKKKKKKKKKKKKKKKKK |
| 989 | Q | 0.963 | 0.97560976 | 1547 | # QQQQQQQQQQQQQQQQQQQQQQQQQQQQQQQQQQQQQQQPQ |
| 990 | M | 0.963 | 0.97560976 | 1548 | # MMMMMMMMMMMMMMMMMMMMMMMMMMMTMMMMMMMMMMMMM |
| 991 | A | 0.963 | 0.97560976 | 1549 | # AAAAAAAAAAAAAAAGAAAAAAAAAAAAAAAAAAAAAAAAA |
| 992 | G | 0.963 | 0.97560976 | 1550 | # GGGGGGGGGGGGGGGSGGGGGGGGGGGGGGGGGGGGGGGGG |
| 993 | D | 1 | 1 | 2900 | # DDDDDDDDDDDDDDDDDDDDDDDDDDDDDDDDDDDDDDDDD |
| 994 | D | 0.963 | 0.97560976 | 1551 | # DDDDGDDDDDDDDDDDDDDDDDDDDDDDDDDDDDDDDDDDD |
| 995 | C | 1 | 1 | 2901 | # CCCCCCCCCCCCCCCCCCCCCCCCCCCCCCCCCCCCCCCCC |
| 996 | V | 1 | 1 | 2902 | # VVVVVVVVVVVVVVVVVVVVVVVVVVVVVVVVVVVVVVVVV |
| 997 | A | 1 | 1 | 2903 | # AAAAAAAAAAAAAAAAAAAAAAAAAAAAAAAAAAAAAAAAA |
| 998 | S | 0.852 | 0.82926829 | 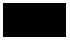 | # GSSGGSGSSSSSSSGSSSSSSSSGSSSSSSSSGSSSSSSSS |
| 999 | R | 1 | 1 | 2904 | # RRRRRRRRRRRRRRRRRRRRRRRRRRRRRRRRRRRRRRRRR |
| 1000 | Q | 1 | 1 | 2905 | # QQQQQQQQQQQQQQQQQQQQQQQQQQQQQQQQQQQQQQQQQ |
| 1001 | D | 1 | 1 | 2906 | # DDDDDDDDDDDDDDDDDDDDDDDDDDDDDDDDDDDDDDDDD |
| 1002 | E | 1 | 1 | 2907 | # EEEEEEEEEEEEEEEEEEEEEEEEEEEEEEEEEEEEEEEEE |
| 1003 | D | 1 | 1 | 2908 | # DDDDDDDDDDDDDDDDDDDDDDDDDDDDDDDDDDDDDDDDD |

Table 2V

| | | | | | |
|---|---|---|---|---|---|
| 1004 | D | 1 | 1 | 2909 | # DDDDDDDDDDDDDDDDDDDDDDDDDDDDDDDDDDDDDDDDD |

Sample#: 200

| | | 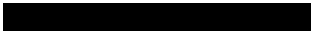 | | |
|---|---|---|---|---|
| Vif | | | Ranking | |
| AA# Cons. | entropy | ratio: Cons. | of entropy | Raw data |
| tc,1 Table 3A-1 | | | | |
| 1M | 0.99 | 0.995 | 2059 | #MMMMMMMMMMMMMMMMMMMMMMMMMMMMMMMMMMMMMMMMMMMMMMMMMMMMMMMMMMMMMMMMMM |
| 2E | 0.99 | 0.995 | 2060 | #EEEEEEEEEEEEEEEEEEEEEEEEEEEEEEEEEEEEEEEEEEEEEEEEEEEEEEEEEEEEEEEEEE |
| 3N | 0.979 | 0.99 | 1887 | #SNNNNNNNNNNNNNNNNNNNNNNNNNNNNNNNNNNNNNNNNNNNNNNNNNNNNNNNNNNNNNNNNN |
| 4R | 0.971 | 0.985 | 1674 | #RRRRRRRRRRRRRRRRRRRKRRRRRRRRRRRRRRRRRRRRRRRRRKRRRRRRRRRRRRRRRRRRRR |
| 5W | 0.99 | 0.995 | 2061 | #WWWWWWWWWWWWWWWWWWWWWWWWWWWWWWWWWWWWWWWWWWWWWWWWWWWWWWWWWWWWWWWWWW |
| 6Q | 0.979 | 0.99 | 1888 | #QQQQQQQQQQQQRQQQQQQQQQQQQQQQQQQQQQQQQQQQQQQQQQQQQQQQQQQQQQQQQQQQQQ |
| 7V | 0.962 | 0.975 | 1357 | #VVVVVVVVVVVVVVVVVVVVVVVVVVVVVVVVAVVVVVVVVVVVVVVVVVVVVVVVVVVVVVVVVV |
| 8M | 0.99 | 0.995 | 2062 | #MMMMMMMMMMMMMMMMMMMMMMMMMMMMMMMMMMMMMMMMMMMMMMMMMMMMMMMMMMMMMMMMMM |
| 9I | 0.962 | 0.975 | 1358 | #IIIIVIIIIIIIIIIIIIIIIIIIIIIIIIIIIIIIIIVIIIIIIVIVIIIIIIIIIIIIIIIIII |
| 10V | 1 | 1 | 2910 | #VVVVVVVVVVVVVVVVVVVVVVVVVVVVVVVVVVVVVVVVVVVVVVVVVVVVVVVVVVVVVVVVVV |
| 11W | 1 | 1 | 2911 | #WWWWWWWWWWWWWWWWWWWWWWWWWWWWWWWWWWWWWWWWWWWWWWWWWWWWWWWWWWWWWWWWWW |
| 12Q | 0.99 | 0.995 | 2063 | #QQQQQQQQQQQQQQQQQQQQQQQQQQQQQQQQQQQQQQQQQQQQQQQQQQQQQQQQQQQQQQQQQQ |
| 13V | 0.971 | 0.985 | 1675 | #VVVVVVVVVVVVVVVVVVVVVVVVVVVVVVVVVVVVVVVVVVVVVVVVVVVVVVVVVVVVVVVVVV |
| 14D | 0.99 | 0.995 | 2064 | #DDDDDDDDDDDDDDDDDDDDDDDDDDDDDDDDDDDDDDDDDDDDDDDDDDDDDDDDDDDDDDDDDD |
| 15R | 0.99 | 0.995 | 2065 | #RRRRRRRKRRRRRRRRRRRRRRRRRRRRRRRRRRRRRRRRRRRRRRRRRRRRRRRRRRRRRRRRRR |
| 16M | 0.99 | 0.995 | 2066 | #MMMMMMMMMMMMMMMMMMMMMMMMMMMMMMMMMMMMMMMMMMMMMMMMMMMMMMMMMMMMMMMMMM |
| 17R | 0.982 | 0.99 | 1907 | #RRRRRRRKRRRRRRRRRRRRRRRRRRRRRRRRRRRRRRRRRRRRRRRRRRRRRRRRRRRRKRRRRR |
| 18I | 1 | 1 | 2912 | #IIIIIIIIIIIIIIIIIIIIIIIIIIIIIIIIIIIIIIIIIIIIIIIIIIIIIIIIIIIIIIIIII |
| 19R | 0.785 | 0.82 | 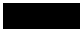 | #RNRRNRRRRRRRRRRKKRKRRRRKRRRRRRRRRRRKRRRSRRRRRRRRRRRRRRRSKKKRKRRRRR |
| 20T | 0.87 | 0.895 | 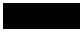 | #TTTTTTTATTTTTTTTTATTTTTTTTTTTTTTATTTTATTTTTTTTTTTTTTTTAATTTTTTTATT |
| 21W | 0.961 | 0.98 | 1333 | #WWWWWWW$WWWWWWWWWWWWWWWWWWWWWWWWWWWWWWWWWWWWWWWWWWWWWWWWWWWWWWWWWW |
| 22K | 0.827 | 0.855 | 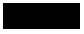 | #KKKKKKKKKKKNKKKKKKKKKKKNKKNKKKKNNKKKKKNKKKKKKKKNKKKHKKNNKKKNKKKKKN |
| 23S | 0.943 | 0.97 | 1169 | #SSSSSSSSSSSSSSSSSSSSSSSSSSSSSSSSSSSSSSSSSSSSSSSSSSSSSSSSSSSSSSSSSS |
| 24L | 1 | 1 | 2913 | #LLLLLLLLLLLLLLLLLLLLLLLLLLLLLLLLLLLLLLLLLLLLLLLLLLLLLLLLLLLLLLLLLL |
| 25V | 1 | 1 | 2914 | #VVVVVVVVVVVVVVVVVVVVVVVVVVVVVVVVVVVVVVVVVVVVVVVVVVVVVVVVVVVVVVVVVV |
| 26K | 0.979 | 0.99 | 1889 | #KKKKKKKKKKKKKKKKKKKKKKKKKKKKKKKKKKKKKKKKKKKKKKKKKKKKKKKKKKKKKKKKKK |
| 27H | 0.908 | 0.92 | 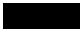 | #HYHHYYHHHHHHHHHHHHHHHHHHHHHHHHHHHHHHHHHHHHHHHHHHHHYHYYHHHHHHHHHHHH |
| 28H | 0.99 | 0.995 | 2067 | #HHHHHHHHHHHHHHHHHHHHHHHHHHHHHHHHHHHHHHHHHHHHHHHHHHHHHHHHHHHHHHHHHH |
| 29M | 0.859 | 0.88 | 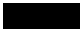 | #MMMMMMMILMMMMMIMMMMMMMIMMMMMMMMIMIIMMMMMIMMMMMMMMMMMMMMMMMMMMMMMMM |
| 30Y | 0.894 | 0.91 | 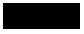 | #YHYYHYYYYYYYYYYYYYYYYYYYYYYYYYYYYHYYYYYYYYYHYYYYYYYHYYYYYYYYYYYYHY |
| 31V | 0.576 | O.23 | 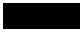 | #IVTIIWIIKIVVVVIIIIIIIIVVIIIIKVSIIIIIIIIRNVIIIIVVIIKIKKIVIIIIIVITVV |
| 325 | 0,974 | 0.985 | 1774 | #SSSSSSSSSSSSSSSSSSSSSSSSSSSSSSSSSSSSSSSSSSSSSSSSSSSSSSSSSSSSSSSSSS |
| 33G | 0.625 | 0.415 | 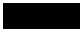 | #GKKKRRRKGGGKRKRKRGRGGGKGGEGKRKGKRGRGRGGKKGGKRKGKKGGRRRKRKKGKKKKRRG |
| 34K | 0.93 | 0.955 | 1016 | #KKKKKKKTKKKKKKKKKKRKKKKKKKKKKKKKKKKKKKKKKKKKKKKKKKKKKKKKKKKKKKKKKK |
| 35A | 0.933 | 0.96 | 1034 | #AAAAAAAAAAAAAAAAAAAAAAAAAAAAAAAAAAAAAAAAAAAAAAAAAAAAATAAAAAAAAAAAA |
| 36R | 0.699 | 0.195 | 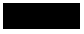 | #KRKKKEKKRKKKTKKKKKKRRRKKKKKKRKKREKTKERTKRKTKKKKKKREKGERKKKRKKKKEKR |
| 370 | 0.65 | 0.69 | 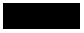 | #REGGKKGRRGGGGGGGGGGKKGGGGGGGKGGGRRGGRGGGGRGGDGGGGGKGERGGGGGRGGERGG |
| 38W | 0.956 | 0.975 | 1303 | #WWWWWW$$WWWWWWWWWWWWWWWWWWWWWWWWWWWWWWWWWWWWWWWWWWWWRWWWWWWWWWWWWW |
| 39P | 0.478 | 0485 | 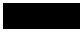 | #SSVFAFFFVACIFFICFFIIIVFVFFSFFFAVVSFVVVCIFFCFCCVFFVFSFFFSFFFVFFSSFI |
| 40Y | 0.99 | 0995 | 2068 | #YYYYYYYYYYYYYYYYYHYYYYYYYYYYYYYYYYYYYYYYYYYYYYYYYYYYYYYYYYYYYYYYYY |
| 41R | 0.8S8 | 0845 | 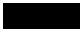 | #KRRRRRRRRRRRRRKRKRRRRKRRRRRRRRRRKRRKKKRRRRRKRKKRRRRRRRRRRRRRRRRKRR |
| 42H | 0.99 | 0995 | 2069 | #HHHHHHHHHHHHHHHHHHHHHHHHHHHHHHHHHHHHHHHHHHHHHHHHHHHHHHHHHHHHHHHHHH |
| 43H | 1 | 1 | 2915 | #HHHHHHHHHHHHHHHHHHHHHHHHHHHHHHHHHHHHHHHHHHHHHHHHHHHHHHHHHHHHHHHHHH |
| tc,1 Table 3A-2 | | | | |
| 1M | 0.99 | 0.995 | 2059 | MMMMMMMMMMMMMMMMMMMMMMMMMMMMMMMMMMMMMMMMMMMMMMMMMMMMMMMMMMMMMMMMMMM |
| 2E | 0.99 | 0.995 | 2060 | EEEEEEEEEEEEEEEEEEEEEEEEEEEEEEEEEEEEEEEEEEEEEEEEEEEEEEEEEEEEEEEEEEE |
| 3N | 0.979 | 0.99 | 1887 | NNNNNNNNNNNNNNNNNNNNNNNNNNNNNNNNNNNNNNNNNNNNNNNNNNNNNNNNNNNNNNNNNNN |
| 4R | 0.971 | 0.985 | 1674 | RRRRRRRRRRRRRRRRRRRRRRRRRRRRRRRRRRRRRRRRRRRRRRRRRRRRRRRRRRRRRRRRRRR |
| 5W | 0.99 | 0.995 | 2061 | WWWWWWWWWWWWWWWWWWWWWWWWWWWWWWWWWWWWWWWWWWWWWWWWWWWWWWWWWWWWWWWWWWW |
| 6Q | 0.979 | 0.99 | 1888 | QQQQQQQQQQQQQQQQQQQQQQQQQQQQQQQQQQQQQQQQQQQQQQQQQQQQQQQQQQQQQQQQQQQ |
| 7V | 0.962 | 0.975 | 1357 | VVVVVVVVVVVAVVVVVVVVVVVVVVVVVVVVVVVVVVVVVAVVVVVVVVVVVVVVVVVVVVVVVVV |
| 8M | 0.99 | 0.995 | 2062 | MMMMMMMMMMMMMMMMMMMMMMMMMMMMMMMMMMMMMMMMMMMMMMMMMMMMMMMMMMMMMMMMMMM |
| 9I | 0.962 | 0.975 | 1358 | IIIIIVIIIIIIIIIIIIIIIIIIIIIIIIIIIIIIIIIIIIIIIIIIIIIIIIIIIIIIIIIIIII |
| 10V | 1 | 1 | 2910 | VVVVVVVVVVVVVVVVVVVVVVVVVVVVVVVVVVVVVVVVVVVVVVVVVVVVVVVVVVVVVVVVVVV |
| 11W | 1 | 1 | 2911 | WWWWWWWWWWWWWWWWWWWWWWWWWWWWWWWWWWWWWWWWWWWWWWWWWWWWWWWWWWWWWWWWWWW |
| 12Q | 0.99 | 0.995 | 2063 | QQQQQQQQQQQQQQQQQQQQQQQQQQQQQQQQQQQQQQQQQQQQQQQQQQQQQQQQQQQQQQQQQQQ |
| 13V | 0.971 | 0.985 | 1675 | VVVVVVVVVVVVVVVVVVVVVVVVVVVVVVVVVVVVVVVVVVVVVVVVVVVVVVVVVVVVVVVVVVV |
| 14D | 0.99 | 0.995 | 2064 | DDDDDDDDDDDDDDDDDDDDDDDDDDDDDDDDDDDDDDDDDDDDDDDDDDDDDDDDDDDDDDDDDDD |
| 15R | 0.99 | 0.995 | 2065 | RRRRRRRRRRRRRRRRRRRRRRRRRRRRRRRRRRRRRRRRRRRRRRRRRRRRRRRRRRRRRRRRRRR |
| 16M | 0.99 | 0.995 | 2066 | MMMMMMMMMMMMMMMMMMMMMMMMMMMMMMMMMMMMMMMMMMMMMMMMMMMMMMMMMMMMMMMMMMM |
| 17R | 0.982 | 0.99 | 1907 | RRRRRRRRRRRRRRRRRRRRRRRRRRRRRRRRRRRRRRRRRRRRRRRRRRRRRRRRRRRRRRRRRRR |
| 18I | 1 | 1 | 2912 | IIIIIIIIIIIIIIIIIIIIIIIIIIIIIIIIIIIIIIIIIIIIIIIIIIIIIIIIIIIIIIIIIII |
| 19R | 0.785 | 0.82 | 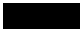 | RRRRRRRRRRRRRRRSKRRRRRRRRRRRRRNRRRSRRRRRNRRRSRRRNRRRRRRRRRRRKRSRRRR |
| 20T | 0.87 | 0.895 | 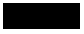 | TTTTTATTTTATTXATTTTTTTTTTTTTTTTTTTTTTTATTTTTTTTTTTTTTATTTTTTTTTTTTT |
| 21W | 0.961 | 0.98 | 1333 | WWWWWWWWWWWWWWWWWWWWWWWWWWWGWWWWWWWWWWWWWWWWWWWWWWWWWWWWWWWWWWWWWWW |
| 22K | 0.827 | 0.855 | 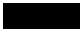 | KKNKKKKNKKKKKKKKKKKKNKKKKKKKNKKKKKKKKKKEKKKKKKKNKKKKKKKKKKKKKKKKKKK |
| 23S | 0.943 | 0.97 | 1169 | SSSSSSSSSSSSSSSSSSSSSRSSSSSSSSSSSSGSSSSSSSSSSSSSSSSSSSSSSSSSSSSSSSS |
| 24L | 1 | 1 | 2913 | LLLLLLLLLLLLLLLLLLLLLLLLLLLLLLLLLLLLLLLLLLLLLLLLLLLLLLLLLLLLLLLLLLL |
| 25V | 1 | 1 | 2914 | VVVVVVVVVVVVVVVVVVVVVVVVVVVVVVVVVVVVVVVVVVVVVVVVVVVVVVVVVVVVVVVVVVV |
| 26K | 0.979 | 0.99 | 1889 | KKKKKKKKKKKKKKKKKKKKKKKKKKKKKKKKKKKKKKKKKKKKKKKKKKKKKKKKKKKKKKKKKKK |
| 27H | 0.908 | 0.92 | 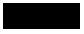 | HHHHHHHHHHHHHYHYHHHHHHHHHHHHHHYHHHHHHHHYHHHHHHHHYHHHHHYHHHHHHHHHHHY |
| 28H | 0.99 | 0.995 | 2067 | HHHHHHHHHHHHHHHHHHHHHHHHHHHHHHHHHHHHHHHHHHHHHHHHHHHHHHHHHHHHHHHHHHH |
| 29M | 0.859 | 0.88 | 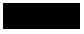 | MMMMMMMIMMMMIXMMMMMMMMMMMMMMMMMMMMMMMMMMMMIMMMMIMMMMMIMMIMMMIMIMMMM |
| 30Y | 0.894 | 0.91 | 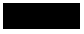 | HYYHYYYYYYYYYHYYYYYYYYHYYYYYYYYYYYYYHYYYYYYYYYYYYYYYYYYYYYYYYYHYYYY |
| 31V | 0.576 | O.23 | 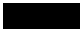 | VVVVIIVCVVKIVXTIVIIVRIVIVKISIIIIITIIIIIVTIIIIIICMIVVVMTIIVIIVIIIIRV |
| 325 | 0,974 | 0.985 | 1774 | SSSSSSSSSSTSSSSSSSSSSSSSSTSSSSSSSSSSSSSSSSSSSSSSSSSSSSSSSSSSSSSSSSS |
| 33G | 0.625 | 0.415 | 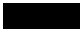 | RRRRRRGRRGGGGKGRGGKKKGGKRGGGRKKRRRGRRGRKEGRGGRRKKGRRGRKGRKGGKRRGRGK |
| 34K | 0.93 | 0.955 | 1016 | KKKKKKKKKKKKKKKKKKKKKKKKKKKKKKKKKKKKKKKKKKKKKKKKKKKKKKKKKKKKKKKKKKK |
| 35A | 0.933 | 0.96 | 1034 | AAAAAAAAAAAGAAAAAAAAAAAAAAAAAAAAAVAAAAAAAAAAAAAAAAAAAAAAAAAAATAAAAA |
| 36R | 0.699 | 0.195 | 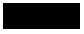 | KKKKKKEKKRRRKXRKKKKKKKKKKRKKKKKKKKKKKKKKKKKKKKKKKKGERRKKKKKKQKKKKKK |
| 370 | 0.65 | 0.69 | 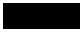 | GGGGGGKNGGKGGGGGGGGGGGGEGKKGGGGGGGGGGGGGRRQKGGGGARKKGGGGGGKKGDGKGGK |
| 38W | 0.956 | 0.975 | 1303 | WWWWWWWWWWWWWWWWWWWWWWWWWWWWWWWWWWWWWWWWWWWWWWWWWWWWWWWWWWWW$WWWWWW |
| 39P | 0.478 | 0485 | 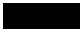 | FVVFVFFVFFGSFIVFFFFFFIIVFWfFFVVSVFFFVSFLFFVSSVFFYVFVVFVFLFSVIVLVFFF |
| 40Y | 0.99 | 0995 | 2068 | YYYYYYYYYYYYYYYYYYYYYYYYYYYYYYYYYYYYYYYYYYYYYYYYYYYYYYYYYYYYYYYYYYY |
| 41R | 0.8S8 | 0845 | 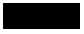 | RRRRRRKRRRRRRRRRRRRRRRRRRRRKRRKRRRRRKRRRRRRRKRRRRRRRRRRRRRRKKRRRRRR |
| 42H | 0.99 | 0995 | 2069 | HHHHHHHHHHHHHHHHHHHHHHHHHHHHHHHHHHHHHHHHHHHHHHHHHHHHHHHHHHHHHHHHHHH |
| 43H | 1 | 1 | 2915 | HHHHHHHHHHHHHHHHHHHHHHHHHHHHHHHHHHHHHHHHHHHHHHHHHHHHHHHHHHHHHHHHHHH |
| tc,1 Table 3A-3 | | | | |
| 1M | 0.99 | 0.995 | 2059 | MMMMMMMMMMMMMMMMMMMMTMMMMMMMMMMMMMMMMMMMMMMMMMMMMMMMMMMMMMMMMMMMMMM |
| 2E | 0.99 | 0.995 | 2060 | EEEEEEEEEEEEEEEEEEEEEEEEEEEEEEEEEEEEEEEEEEEEEEEEEEEEEEEEEEEEEEEEEEE |
| 3N | 0.979 | 0.99 | 1887 | NNNNNNNNNNNNNNNNNNNNNNNNNNNNNNNNNNNNNNNNNNNNNNNNNNNNNNNNNNNNNNNNNNN |
| 4R | 0.971 | 0.985 | 1674 | RRRRRRRRRRRRRRRRRRRRRRRRRRRRRSRRRRRRRRRRRRRRRRRRRRRRRRRRRRRRRRRRRRR |
| 5W | 0.99 | 0.995 | 2061 | WWWWWWWWWWWWWWWWWWWWWWWWWWWWWWWWWWWWWWWWWWWWWWWWWWWWWWRWWWWWWWWWWWW |
| 6Q | 0.979 | 0.99 | 1888 | QQQQQQQQQQQQQQQQQQQQQQQQQQQQQQQQQQQQQQQQQQQQQQQQQQQQQQQQQQQQQQQQQQQ |
| 7V | 0.962 | 0.975 | 1357 | VVVVVVVVVVVVVVVVVVVVVVVAVVVVVVAVVVVVVVVVVVVVVVVVVVVVVVVVVVVVVVVVVVV |
| 8M | 0.99 | 0.995 | 2062 | MMMMMMMMMMMMMMMMMMMMMMMMMMMMMMMMMMMMMMMMMMMMMMMMMMMKMMMMMMMMMMMMMMM |
| 9I | 0.962 | 0.975 | 1358 | IIIIIIIIIIIIIIIIIIIIIIIIIIIIIIIIIIIIIIIIIIIIIIIIIIIIIIIIIIIIIIIIIII |
| 10V | 1 | 1 | 2910 | VVVVVVVVVVVVVVVVVVVVVVVVVVVVVVVVVVVVVVVVVVVVVVVVVVVVVVVVVVVVVVVVVVV |
| 11W | 1 | 1 | 2911 | WWWWWWWWWWWWWWWWWWWWWWWWWWWWWWWWWWWWWWWWWWWWWWWWWWWWWWWWWWWWWWWWWWW |
| 12Q | 0.99 | 0.995 | 2063 | QQQQQLQQQQQQQQQQQQQQQQQQQQQQQQQQQQQQQQQQQQQQQQQQQQQQQQQQQQQQQQQQQQQ |
| 13V | 0.971 | 0.985 | 1675 | VVVVVVVVVVVVVVVVVIVVVVVVVVVVVVVVVVVVVVIVVVVVVVVVVVVVVVVVVVVVVVVVVVV |
| 14D | 0.99 | 0.995 | 2064 | DDDDYDDDDDDDDDDDDDDDDDDDDDDDDDDDDDDDDDDDDDDDDDDDDDDDDDDDDDDDDDDDDDD |
| 15R | 0.99 | 0.995 | 2065 | RRRRRRRRRRRRRRRRRRRRRRRRRRRRRRRRRRRRRRRRRRRRRRRRRRRRRRRRRRRRRRRRRRR |
| 16M | 0.99 | 0.995 | 2066 | MMMMMMMMMMMMMMMMMMMMMMMMMMIMMMMMMMMMMMMMMMMMMMMMMMMMMMMMMMMMMMMMMMM |
| 17R | 0.982 | 0.99 | 1907 | RRRRRRRRRRRRRRRRRRRRRRRRRRRRRRRRRRRRRRRRRRRRRRRRRRRRRRRRRRRRRRRRRRR |
| 18I | 1 | 1 | 2912 | IIIIIIIIIIIIIIIIIIIIIIIIIIIIIIIIIIIIIIIIIIIIIIIIIIIIIIIIIIIIIIIIIII |
| 19R | 0.785 | 0.82 | 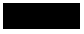 | RRRRRRRRRRRKRRRRRRRRRRRKRKKKKKRRSRNRRSRRSRSRRRRRRRRRRRRNRRRRRRRRRRK |
| 20T | 0.87 | 0.895 | 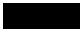 | TTTANITTTTTTAATTTTTTTTTTTTTTTTATTTTTTTTTTATTTTTTTTTTTTTTTTTATTTTTTT |
| 21W | 0.961 | 0.98 | 1333 | WWWWMWWWWWWWWWWWW$WWWWWWWWWGWWWWWWWWWWWWWWWWWWWWWWWWWWWWWWWWWWWWWWW |
| 22K | 0.827 | 0.855 | 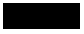 | NKKKEKKKKKKKKKKKKKKNKKKKKKKKKHKNKKKKKKKKKKKKNKKKNNTKKKKKKNKKIKKKKKK |
| 23S | 0.943 | 0.97 | 1169 | SSSSPSSSSSSSSSSSSSSSSSSSSSSSSSSSRSSSSSSSSSSSSSSSSSSSSSSRSSSSSSSSSSI |
| 24L | 1 | 1 | 2913 | LLLLLLLLLLLLLLLLLLLLLLLLLLLLLLLLLLLLLLLLLLLLLLLLLLLLLLLLLLLLLLLLLLL |
| 25V | 1 | 1 | 2914 | VVVVVVVVVVVVVVVVVVVVVVVVVVVVVVVVVVVVVVVVVVVVVVVVVVVVVVVVVVVVVVVVVVV |
| 26K | 0.979 | 0.99 | 1889 | KKKKKKKKKKKKKKRKKKKKKKKKKKKKKKKKKKKKKKKKKKKKKKKKKKKKKKKKKKKKKKKKKKK |
| 27H | 0.908 | 0.92 | 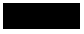 | HHHHHHHHHHHHHHHHHHHHHHHHHHHHHHHHHHHHHHHHYHHHHHHHHHHHHHHHHHHHHHHHHHY |
| 28H | 0.99 | 0.995 | 2067 | HHHHHHHHHHHHHHHHHHHHHHHHHHHHHHHHHHHHHHHHHHHHHHHHHHHHHHHHHHHHHHHHHHH |
| 29M | 0.859 | 0.88 | 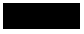 | MMIIMMMMMMMMMMMIMMMMMMIMMMMMMIMMMMMMMMMMMMMMMMMMMMVMMMMMMMMMMIMMMMM |
| 30Y | 0.894 | 0.91 | 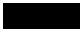 | YYHHYYYYYYYYYYYYYYYYYYYYYYYYYYYYYFYYYYYYYYYYYYYHYYYYYYYYYYYYYHYYYYH |
| 31V | 0.576 | O.23 | 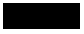 | VIIIVIIIVIIIKIVIIIIIIIIIIRRRRIICIIIIVVIITIIVIIIIKVVVIIIIIITIIIIIVTV |
| 325 | 0,974 | 0.985 | 1774 | SSSSSSSSSSSSSSSSSSSSSSSSSSSSSSSSSSSSSSTSSSSSSSSSSSSSSSSSSSSSSSSSSSS |
| 33G | 0.625 | 0.415 | 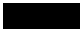 | KGKKRGGGRGGKKKKKGGGGGGRGGGGGGEGKRRKGGGGKKKKGGRKGRKKGRKKRGGGGRGGKKGG |
| 34K | 0.93 | 0.955 | 1016 | KKKKKKKKKKKKKKKKKKKKKKKKKRRRRKKKKKKKKKKKRKKKKKKKKKKKKKKKKKKKKKKKKXR |
| 35A | 0.933 | 0.96 | 1034 | AAAAAAAAAAAAAAAAAAAAAATATVAAAAAAAAAAAAAAAAAAAAAAAAAAAAAAAAAAAAAAAAT |
| 36R | 0.699 | 0.195 | 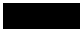 | KRKKKKEKKKKKEKKRRRRKKKKKRRRRRKKNKKKKRRRKRKRKRKKKKKRKKKKKKKKRKRKKKKK |
| 370 | 0.65 | 0.69 | 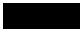 | GGEKGGREGRGERGEGGGGGGKGKGEKEEGRGGGGGGAGGGGKGGGGRGGGKGGGDKGGGGRGGGGE |
| 38W | 0.956 | 0.975 | 1303 | WWWWWWWWWWWWWWWWWWWWWWWWWWWWWWWWWWWWWWWWWWWWWWWWWWWWWWRWWWWWWWWWWWW |
| 39P | 0.478 | 0485 | 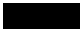 | VCVSLFFFFFCVFLSLFFVVFVVSFFFFFITVFSFFFSLFFFFFFFFLYVFFFFFFVIIFFISSFLR |
| 40Y | 0.99 | 0995 | 2068 | YYYYYYYYYYYYYYYYYYYYYYYYYYYYYYYYYYYYYYYYYYYYYYYYYYYYYYYYYYYYYYYYYYY |
| 41R | 0.8S8 | 0845 | 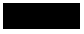 | RRRKRKKRRKRRRKRRRRRRRRRRRRRRRKRKRRKRRRRRRRRRRRRRRKRRRRRRKKRRRKRRRRR |
| 42H | 0.99 | 0995 | 2069 | HHHHHHHHHHHHHHHHHHHHHHHHHHHHHHHHHHHHHHHHHHHHHHHHHHHHHHHHHHHHHHHHHHH |
| 43H | 1 | 1 | 2915 | HHHHHHHHHHHHHHHHHHHHHHHHHHHHHHHHHHHHHHHHHHHHHHHHHHHHHHHHHHHHHHHHHHH |
| tc,1 Table 3B-1 | | | | |
| 44Y | 0.982 | 0.99 | 1908 | #YYYYYYYYYYYYYYYYYYYYYYYYYYYYYYYYYYYYYYYYYYYYYYYYYYYYYYYYYYYYYYYYYY |
| 45E | 0.935 | 0.955 | 1057 | #EEEEEEDEEEEEEEEEDEEEEEEEEEEEEEEEEEEEEEEEEEEEEEEEEEEEEEEEEEEEEEEEEE |
| 46S | 0.89 | 0.92 | 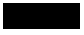 | #SSNSSSSSSSSNSSSNSSSSSSSSSSSSSSSSRSSNSSSSSSSSSSSSSSSSSSSSSSSSSSSSSS |
| 47P | 0.687 | 0.095 | 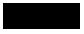 | #TTTTTTTTTTTTHTTLTNTTTTTTTTTPTHTIPTTTTLTTTTTTITTSTTTPTTTTTTTITLTTTT |
| 48H | 0.812 | 0.74 | 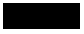 | #NNHHHHHHHHNHHHHHHHNNNNHNNHNHHHHNHHHHNHHNNHHNNHHHHNHHHHHHHHHHNHHNHN |
| 49P | 1 | 1 | 2916 | #PPPPPPPPPPPPPPPPPPPPPPPPPPPPPPPPPPPPPPPPPPPPPPPPPPPPPPPPPPPPPPPPPP |
| 50R | 0.827 | 0.78 | 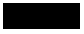 | #RRRRRRKKRRRKKKKRKRRRRRKRRRRKRRRRRRRRRRKRRRRRRRRKRRRRRRKRRRKRRRRRRR |
| 51I | 0.847 | 0.885 | 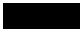 | #VIIIIIILIIIIIIKIIITIIIIITIIIIIIIVIVIIIIIIIIIIIIIIIIIIIIIIIIKIIIVIV |
| 52S | 0.957 | 0.975 | 1317 | #SSSSSSSSSSSSSSSSSSSSSSSSSSSSSSSSSSSSSSSSSSSSGSSSSSSSSSSSSSSSSSSSSS |
| 53S | 0.969 | 0.985 | 1666 | #SSSSSSSSSSSSSSSGSSSSSSSSSSSSSSSSSSSSSSSSSSSSSSSSSSSSSSSSSSSSSSSSSS |
| 54E | 0.961 | 0.98 | 1334 | #EEEEEEEGEEEEEEEEEEEEEEEEEEEEEEEEEEEEEEEEEEEEEEEEEEEEEEEEEEEEEEEEEE |
| 55V | 0.979 | 0.99 | 1890 | #VVVVVVVVVVVVVVVVVVVVVVVVVVVVVVVVVVVVVVVVVVVVVVVVVVVVVVVVVVVVVVVVVV |
| 56H | 0.939 | 0.965 | 1133 | #HHHHHHHHHHHHHHHHHHHHHHHHHHHHHHHHYHHHHHHHHHHHHHHHHHHHHHHHHHHHHHHHHH |
| 57I | 1 | 1 | 2917 | #IIIIIIIIIIIIIIIIIIIIIIIIIIIIIIIIIIIIIIIIIIIIIIIIIIIIIIIIIIIIIIIIII |
| 58P | 1 | 1 | 2918 | #PPPPPPPPPPPPPPPPPPPPPPPPPPPPPPPPPPPPPPPPPPPPPPPPPPPPPPPPPPPPPPPPPP |
| 59L | 0.941 | 0.965 | 1143 | #LLLLLLLLLVLLLLLLLLLLLLLLLLLLLLLLLLLLLLLLLLLLLLLLLLLLLLLLLLVLLLLLLL |
| 60G | 0.931 | 0.96 | 1026 | #GGGGGGGGGRGGGEGGGGGGGGGGGGGGGGGGGGGGGGGGGGGGGGGGGGGGGGGGGGGGGGGGGG |
| 61D | 0.796 | 0.78 | 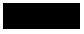 | #DDDDDEDDEEEDEEEPDDDDDDDDEDDDEDEDDDDDDDDDDDDDDDEDEEDDDDDEEDDDDDDDDD |
| 62A | 0.875 | 0.93 | 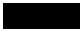 | #ADAAAAAAAAAAAAAAAAAAAAAAAAAAAAAAAAAAAAAAAAAAAAAAAAAAAAAAAAGAAAAAAA |
| 63R | 0.628 | 0.515 | 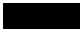 | #KKRKTIRKRKKRREKKRRKKKTRRKRKRRRKKKRRKTKKKRRRKKKRRRRRRKKKRRRRKRRKRKR |
| 64L | 0.947 | 0.97 | 1217 | #LLLLLLLLLLLLLLLLLLLLLLLLLLLLLLLLLLLLLLLMLLLLLLLLLLLLLLLMLLLLILLLLL |
| 65V | 0.951 | 0.97 | 1245 | #VVVVVVVVVVVVVVIVVVVVVVVIVVVVVVVVVVVVVVVVVVVVVVVVVVVVVVVVVVVVVVVVVV |
| 66I | 0.823 | 0.77 | 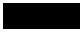 | #IIVIVIVIIIIIIIIIIVIIVVIIIIVIIIIVIIIVIIIIVIIIIIIIIIIIIIIIIIIIIIIIII |
| 67T | 0.81 | 0.87 | 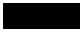 | #KTTTTTTPTTVTTTTTTTTTATTTTTTIITTTTTTSTTRTTTTTTTITTTTTTKTTTTTTTTTTTT |
| 68T | 0.99 | 0.995 | 2070 | #TTTTTTTPTTTTTTTTTTTTTTTTTTTTTTTTTTTTTTTTTTTTTTTTTTTTTTTTTTTTTTTTTT |
| 69Y | 0.979 | 0.99 | 1891 | #YYYYYYYYYYYYYYYYYFYYYYYYYYYYYYYYYYYYYYYYYYYYYYYYYYYYYYYYYYYYYYYYYY |
| 70W | 0.964 | 0.98 | 1556 | #WWWW$WWYWWWWWWWWWWWWWWWWWWWWWWWWWWWWWWWWWWWWWWWWWWWWWWWWWWWWWWWWWW |
| 71G | 0.99 | 0.995 | 2071 | #GNGGGGGGGGGGGGGGGGGGGGGGGGGGGGGGGGGGGGGGGGGGGGGGGGGGGGGGGGGGGGGGGG |
| 72L | 0.979 | 0.99 | 1892 | #LLLLLLLLLLLLLLLLLLLLLLLLLLLLLLLLLLLLLLLLLLLLLLLLLLLLLLLLLLLLLLLLLL |
| 73H | 0.908 | 0.94 | 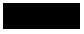 | #HHHHHNHHHHHHHHHHHHHHHHNHHHHHHHHHHHLHHHHHHHHHHQHHHHHHHHHHHHHQHHHHHH |
| 74T | 0.961 | 0.98 | 1335 | #TTTTTTTTTTTTTTTTTTTTTTTTTTTTTTTTTTTTTTTTTTTTTTTTTTTTTTTTTTTTTTTTTT |
| 75G | 0.99 | 0.995 | 2072 | #GGGGGGGGGGGGGGGGGGGGGGGGGGGGGGGGGGGGGGGGGGGGGGGGGGGGGGGGGGGGGGGGGG |
| 76E | 0.979 | 0.99 | 1893 | #EEEEEEEDEEEEEEEEEEEEEEEEEEEEEEEEEEEEEEEEEEEEEEEEEEEEEEEEEEEEEEEEEE |
| 77R | 0.951 | 0.97 | 1246 | #RRRRRRRTRRRRRRRRRRRRRRRRRRRRRRRKRRRRRRRRKRRRRRRRRRRRRRRRRRRRRRRRRR |
| 78D | 0.831 | 0.855 | 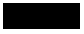 | #DDDDDDHDDDDDDNDDDDDDDDDDDDDDDDDDDDEDDDDDDDEDDDDDDDDDDDDDDDDDDDDDED |
| 79W | 0.982 | 0.99 | 1909 | #WWWWWWWRWWWWWWWWWWWWWWWWWWWWWWWWWWWWWWWWWWWWWWWWWWWWWWWWWWWWWWWWWW |
| 80H | 0.99 | 0.995 | 2073 | #HHHHHHHHHHHHHHHHHHHHHHHHHHHHHHHHHHHHHHHHHHHHHHHHHHHHHHHHHHHHHHHHHH |
| 81L | 0.971 | 0.985 | 1676 | #LLLLLLLFLLLLLLLLLLLLLLLLLLLLLLLLLLLLLLLLLLLLLLLLLLLLLLLLLLLLLLLLLL |
| 82G | 0.979 | 0.99 | 1894 | #GGGGGGGGGGGGGGGGGGGGGGDGGGGGGGGGGGGGGGGGGGGGGGGGGGGGGGGGGGGGGGGGGG |
| 83Q | 0.956 | 0.975 | 1304 | #QQQQQQQQQQQQQQQQQQQQQQQQQQQQQQQQQQQQQQQQQQNQQQQQQQQQQQQQQQQQQQQQQQ |
| 84G | 0.979 | 0.99 | 1895 | #GGGGGGGRGGGGGGGGGGGGGGGGGGGGGGGGGGGGGGGGGGGGGGGGGGGGGGGGGGGGGGGGGG |
| 85V | 0.917 | 0.94 | 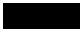 | #AVVVVVVVVVVVVVVVVVVVVVAVVVVVVVVVVVVVVVVVVVVVVVVVVVVVVVVVVVVVVAVAVV |
| 86S | 0.99 | 0.995 | 2074 | #SSSSSSSSSSSSSSSSSSSSSSSSSSSSSSSSSSSSSSSSSSSSSSSSSSSSSSSSSSSSSSSSSS |
| 87I | 0.943 | 0.965 | 1170 | #IIIIIIIIIIIIIIIIIIIIIIIIIIIIIIIIIIIIIIIIIIIIIIIIIIIIIIIIIIIIIIIIII |
| 88E | 0.971 | 0.985 | 1677 | #EEEEEEEEEEEEEEEEEEEEEEEEEEEEEEEEEEEEEEEEEEEEEEEEEEEEEEEEEEEEEEEEEE |
| 89W | 0.979 | 0.99 | 1896 | #WWWWWWW$WWWWWWWWWWWWWWWWWWWWWWWWWWWWWWWWWWWWWWWWWWWWWWWWWWWWWWWWWW |
| 90R | 0.964 | 0.98 | 1557 | #RRRRRRRGRRRRRRRRRRRRRRRRRRRRRRRRRRRRRRRRRRRRRRRRRRRRRRRRRRRRRRRRRR |
| 91K | 0.863 | 0.91 | 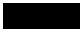 | #KTKKRKKKKKKEKKRKKKKTTKKTKKKKKKKKKKKKKKKKKKKTEKKKKKKKKKKRKQKKEKKKKK |
| tc,1 Table 3B-2 | | | | |
| 44Y | 0.982 | 0.99 | 1908 | YYYYYYYYYYYYYYYYYYYYYYYYYYYYYYYYYYYYYYYYYYYYFYYYYYYYYYYYYYYYFYYYYYY |
| 45E | 0.935 | 0.955 | 1057 | EEEEEEEEEEDEEEEEEEEEEEEEEEEEEDEEEEEEEEEEDEEEEEEEEDEQEEEEEEEEEEEEEEE |
| 46S | 0.89 | 0.92 | 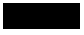 | SSSSNSSSSSSSSSNSSSSSSSSSSSSSSNNNSSSSSSSSSSSSSSSSSSSSSSNSSSSNSSNSSSS |
| 47P | 0.687 | 0.095 | 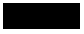 | TTITTTTTTPPTTTTTTTTPTAITTLTTTTTTTITTTNPPNTTTPTTSTITTTTPTTTTTPTTTTTT |
| 48H | 0.812 | 0.74 | 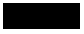 | NHNHHHNNHHHNHHHHHHHHHNNHHHHHHHHHHNHNHHHHHHHHHHHHHHHHHHHNHHHHHHNHHHH |
| 49P | 1 | 1 | 2916 | PPPPPPPPPPPPPPPPPPPPPPPPPPPPPPPPPPPPPPPPPPPPPPPPPPPPPPPPPPPPPPPPPPP |
| 50R | 0.827 | 0.78 | 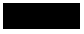 | RRRRRRRRRRKRRRRRRRKRKRRRRRRRKKRKRRRRRRRRRRRRRRKKKKRRRRRRRRRRKRRRRRR |
| 51I | 0.847 | 0.885 | 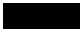 | IIIIIIIVIIIIIIIIIIIIIIIIIIIIIIIIIIIIIIVIIIIIITIIIIIIIIIVIIIIIIIIIII |
| 52S | 0.957 | 0.975 | 1317 | SSSSSSSSSSSSSSSSSSSSSSSSSSSSSSSSSSSSSSGSSSSSSSSSSSSSXSSSSSSSSSSGSSS |
| 53S | 0.969 | 0.985 | 1666 | SSSSSSSSSSSSSSSSSSSSSSSSSSSSSSSSSSSSSSSSSSSSSSSSSSSSSSSSSSSSSSSSSSS |
| 54E | 0.961 | 0.98 | 1334 | EEEEEEEEEEEEEEEEEEEEEEEEEEEEEEEEEEEEEEEEEEEEEEEEEEEEEEEEEEEEEEEEEEE |
| 55V | 0.979 | 0.99 | 1890 | VVVVVVVVVVVVVVVVVVVVVVVVVVVVVVVVVVVVVVVVVVVVVVVVVVVVVVVVVVVVVVVVVVV |
| 56H | 0.939 | 0.965 | 1133 | HHHHHHHHHHHHHHHHHHHHHHHHHHHHHHHHHHHHNHHHHHHHHHHHHHHHHHHHHHHHHHHHHHH |
| 57I | 1 | 1 | 2917 | IIIIIIIIIIIIIIIIIIIIIIIIIIIIIIIIIIIIIIIIIIIIIIIIIIIIIIIIIIIIIIIIIII |
| 58P | 1 | 1 | 2918 | PPPPPPPPPPPPPPPPPPPPPPPPPPPPPPPPPPPPPPPPPPPPPPPPPPPPPPPPPPPPPPPPPPP |
| 59L | 0.941 | 0.965 | 1143 | LLLLLLLLLLLLLLLLLILLLLLLLLLLVLLLLLLLLLLLLLLLLLLLLLVLLLLLLLLLLLLLLLL |
| 60G | 0.931 | 0.96 | 1026 | GGGGGGGGGGGGGGGGGGGGGGGGGGGGGGKGGGGGGGGGGGGGGKGGGGGGGGGGGGGGGGGGGGG |
| 61D | 0.796 | 0.78 | 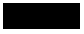 | DDDDDDDENDDEDDEDDDEDDDDDEDDDEKDDDDDDDEDDDEDEEDDDDDEEDDDDDDEEDDDDDDD |
| 62A | 0.875 | 0.93 | 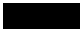 | GAAAAAAAAAAAAAXAAAAAAAAAAAAAAAAAAAAAAAAATAAASAAAAAAASEAAAAAAAAAAAAA |
| 63R | 0.628 | 0.515 | 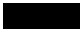 | KKRKRKRKKRKKRRKRKKRRRRRKRKKRRSRKKRKRRTKRRRRRRKKREKRRKKRRRTRRRKRKKRT |
| 64L | 0.947 | 0.97 | 1217 | LLLLLLLLLLLLLLLLLLLLLLLLLLLLLLLLLLLLLLLLLLLLLLLLLLLLLLLLMLLLLLLLLLL |
| 65V | 0.951 | 0.97 | 1245 | VVVVVVVVVVVVVVVVVVVVVVVVVVVVVVVVVVVVVVVVVVVVVVVVVVVVVVVVVVVVVIIVVVV |
| 66I | 0.823 | 0.77 | 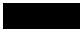 | IIIIVIIIIIIIIIIVIIIVIIIIVIVVIVIIIIIIVIIIVIIIIIIIVIIIVVIIIVVIVIIIIIV |
| 67T | 0.81 | 0.87 | 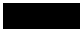 | TTTTTTTTTTTITTATTTTTTTTITTTTTTTITTTTTTTTTITTTTITTTTTTTTTTTTTIKTTITI |
| 68T | 0.99 | 0.995 | 2070 | TTTTTTTTTTTTTTTTTTTTTTTTTTTTTTTTTTTTTTTTTTTTTTTTTTTTTTTTTTTTTTTTTTT |
| 69Y | 0.979 | 0.99 | 1891 | YYYYYYYYYYYYYYYYYYYYYYYYYYYYYYYYYYYYYYYYYYYYYYYYYYYYYYYYYYYYYYYYYCY |
| 70W | 0.964 | 0.98 | 1556 | WWWWWWWWWWWWWWWWWWWWWWWWWWWWWWWWWWWWWWWWWWWWWWWWWWWWWWWWWWWW$WWWWWW |
| 71G | 0.99 | 0.995 | 2071 | GGGGGGGGGGGGGGGGGGGGGGGGGGGGGGGGGGGGGGGGGGGGGGGGGGGGGGGGGGGGGGGGGGG |
| 72L | 0.979 | 0.99 | 1892 | LLLLVLLLLLLLLLLLLLLLLLLLLLLLLLLLLLLLLLLLLLLLLLLLLLLLLLLLLLLLLLLLLLL |
| 73H | 0.908 | 0.94 | 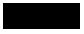 | HHHHHHHHHHHHHHHHHHHHHHHHHHHHHHHHHHHHHHHHHHHHHHHHHHQHHQHHHHHHHHHHHQH |
| 74T | 0.961 | 0.98 | 1335 | TTKTTTTTTTTTTTTTTTTTTTTTTTTTTTTTTTTTTTTTTTTTTTTTTTTTTPTTPTTTTTTTTTT |
| 75G | 0.99 | 0.995 | 2072 | GGGGG$GGGGGGGGGGGGGGGGGGGGGGGGGGGGGGGGGGGGGGGGGGGGGGGGGGGGGGGGGGGGG |
| 76E | 0.979 | 0.99 | 1893 | EEEEEKEEEEEEEEEEEEEEEEEEEEEEEEEEEEEEEEEEEEEEEEEEEEEEEEEEEEEEEEEEEEE |
| 77R | 0.951 | 0.97 | 1246 | RRRRRRRRRRRRRRRRRRRRRRRRRRRRRRRRRRRRRRRRRRRRRRRRRRRRRRRRRRRRRRRRRRR |
| 78D | 0.831 | 0.855 | 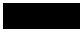 | DDDDDDDDDDDDDDDDDDDDDDDDEDDDDDDDDDDDDDDDEDEDDDDDDDDDDDDDDEDDDDDDDED |
| 79W | 0.982 | 0.99 | 1909 | WWWWWWWWWWWWWWWWWWWWWWWWWWWWWWWWWWWWWWWWWWWWWWWWWWWWWWWWWWWWWWWWWRW |
| 80H | 0.99 | 0.995 | 2073 | HHHHHHQHHHHHHHHHHHHHHHHHHHHHHHHHHHHHHHHHHHHHHHHHHHHHHHHHHHHHHHHHHHH |
| 81L | 0.971 | 0.985 | 1676 | LLLLLLLLLLLLLLLLLLLLLLLLLLLLLLLLLLLLLLLLLLLLLLLLLLLLLLLLLLLLLLLLLLL |
| 82G | 0.979 | 0.99 | 1894 | GGGGGGGGGGGGGGGGGGGGGGGGGGGGGGGGGGGGGGGGGGGGGGGGGGGGGGGGGGGGGGGGGGG |
| 83Q | 0.956 | 0.975 | 1304 | QQQQQQHQQQQQQQQQQQQQQQQQQQQQQQQQQQQQQQQQQQQQQQQQQQQQQQQQQQQQQQQQQQQ |
| 84G | 0.979 | 0.99 | 1895 | GGGGGGGGGGGGGGGGGGGGGGGGGGGGGGGGGGGGGGGGGGGGGGGGGGGGGGGGGGGGSGGGGGG |
| 85V | 0.917 | 0.94 | 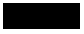 | VVVVVVVAVVVVVVVVVVVVVVVVVVVVVVVVVVVVVVVVVVVVVVVVVVVVVVVVVVVVVVVVVVA |
| 86S | 0.99 | 0.995 | 2074 | SSSSSSSSSSSSSSSSSSSSSSSSSSSSSSSSSSSSSSSSSSSSSSSSSSSSSSSSSSSSSSSSSSS |
| 87I | 0.943 | 0.965 | 1170 | IMIIIVIIIIIIIIIIIIIIIIIIIIIIIIIIIIIIIIIIIIIIIIIVIIIIIIIVIIIIIIIIIII |
| 88E | 0.971 | 0.985 | 1677 | EEEEEEEEEEEEEEEEEEEEEEEEEEEEEEEEEEEEEEEEEEEEEEEEEEEEEEEEEEEEEEEEEEE |
| 89W | 0.979 | 0.99 | 1896 | WWWWWWWWLWWWWWWWWWWWWWWWWWWWWWWWWWWWWWWWWWWWWWWWWWWWWWWWWWWWWWWWWWW |
| 90R | 0.964 | 0.98 | 1557 | RRRRRKRRRRRRRRRRRRRRRRRRRRRRRRRRRRRRKRRRRRRRRRRRRRRRRRRRRRRRRRRRRRR |
| 91K | 0.863 | 0.91 | 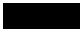 | RKKKKKKKKKKKKKKKKKKKKKKKKKKKKKEKKKKKKKKKKKKKKKKKKKKKKKKKKKTKKKKKKKK |
| tc,1 Table 3B-3 | | | | |
| 44Y | 0.982 | 0.99 | 1908 | YYYYYYYYYYYYYYYYYYYYYYYYYYYYYYYYYYYYYYYYYYYYYYYYYYYYYYYYYYYYYYYYYYY |
| 45E | 0.935 | 0.955 | 1057 | EEEEEEEEEEEEEEEEEEEEEEEEEEEEEEEEEEEEEEEEEDEEEEEDEEEEEEEEEEEEEEEEEEE |
| 46S | 0.89 | 0.92 | 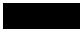 | SSSSSSSSSSSSSSSGSSSSSSSSSSSSISSSSSSSSSSSSSSSSSSSSSSSSSSSSSSSSNSSSSS |
| 47P | 0.687 | 0.095 | 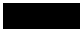 | INTTTTTTNTPTTTTTPPTTTPTTTTTTTNTITTSPTTTTTTTTTPPRTNTTTTTTMTTPTTTTTTT |
| 48H | 0.812 | 0.74 | 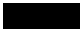 | NHNNHHNNHNHHHHHHHHNNHHNNHHHHHNNNNHNHHHNHHHHHHHHHHNHHHHHNNNHHHHNHHHH |
| 49P | 1 | 1 | 2916 | PPPPPPPPPPPPPPPPPPPPPPPPPPPPPPPPPPPPPPPPPPPPPPPPPPPPPPPPPPPPPPPPPPP |
| 50R | 0.827 | 0.78 | 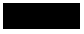 | KKRRKRRRRRRRRRKKRRRKRRKRRKKKKKRRRRRRRRRKKKRRRRRKKRKRRRRKRRRRKRRRRRR |
| 51I | 0.847 | 0.885 | 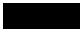 | IIIIIIIIIIVIIIIIIIIIIITIIIIIIIVIIVIIIIIIVIIIIIIIIIIIIVIITVIIIVIIIII |
| 52S | 0.957 | 0.975 | 1317 | SSSSSGSSSSSSSSSSSSSSSSSSSSSSSSSSSSSSSSSSSSSSSSSSSSSSSSSSSSSSSSSSSSS |
| 53S | 0.969 | 0.985 | 1666 | SSSSSSSSSSSSSSSSSSSSSSSSPYSSSSSSSSSSSSSSSSSSSSSSSSSSSSSSSSSSSSSSSSS |
| 54E | 0.961 | 0.98 | 1334 | ETEEEEEEEEAEEEEEEEEEEEEEEEEEEEEEEEEEEEEEEEEEAEEEEEEEEEEEEEEEEEEEEEE |
| 55V | 0.979 | 0.99 | 1890 | VVVVVVVVVVVVVVVVVVVVVVVVV$VVVVVVVVVVVVVVVVVVVVVVVVIVVVVVVVVVVVVVVVV |
| 56H | 0.939 | 0.965 | 1133 | HHHHHHHHHHHHHHQHHHHHHHHHHHHHHHHHHHHHHHYHHHHHYHHHHHHHHHHHHQHHHHHHHYH |
| 57I | 1 | 1 | 2917 | IIIIIIIIIIIIIIIIIIIIIIIIIIIIIIIIIIIIIIIIIIIIIIIIIIIIIIIIIIIIIIIIIII |
| 58P | 1 | 1 | 2918 | PPPPPPPPPPPPPPPPPPPPPPPPPPPPPPPPPPPPPPPPPPPPPPPPPPPPPPPPPPPPPPPPPPP |
| 59L | 0.941 | 0.965 | 1143 | LLLLLLLLLLLLVLLLLLLLLLLLLLLLLLLLLLLLLLLLLLLLLLLLLLLLPLLLLLLLLLLLLLL |
| 60G | 0.931 | 0.96 | 1026 | GGGGGRGGGGGGGGGGGGGGGRGGGGGGGGGGGRGGGGGGGGGGGGGGEGGGGGGGGGGGGGGGGGG |
| 61D | 0.796 | 0.78 | 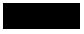 | DDDEDEEDDEDDEDDDDDDDDDDDEVDDDEDEDDDDDDDDDDDDDEDDDDDDDDDDDDDDDDDEDDE |
| 62A | 0.875 | 0.93 | 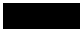 | AAAAAAAAAASAAAAAAAAAAAAAAVAAAAAAAAAAAAAAAAAAAATAAEAAEAAAAAAAAAAGAAA |
| 63R | 0.628 | 0.515 | 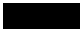 | KRKLKKRRRSRKRTRKRRRKRKRRRKRRRKERREIRRGRKRRKRRRKTKKRNRKRKRRRKRMKKSKR |
| 64L | 0.947 | 0.97 | 1217 | LLLLLLLMLLLLLLLLLLVLLLLLLLLLLLLLLLLLLLLLLLLLLLLLLLLLLLLLLLLLLLLLLLL |
| 65V | 0.951 | 0.97 | 1245 | VVVVVVVVVVVVVVVVVAVIVVVVVVVVVVVVVVVVVVVVVVVVVVVVVVVVVVVVVVVVVVVVVVV |
| 66I | 0.823 | 0.77 | 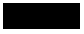 | IVIVIIVIIIIIIIIIIIVIVVVVVVVVVIIIIIIIIIIVIIIIVIVVIIVIIIIIIIIIIIVIVII |
| 67T | 0.81 | 0.87 | 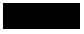 | TTATKTTTTTTTTTTTTTTKTTTTTTTTTATTTTTTTTTTTTTTTTITTTTTTTTTKTTTTTTTTTT |
| 68T | 0.99 | 0.995 | 2070 | TTTTTTTTTTTTTTTTTTTTTTTTTTTTTTTTTTTTTTTTTTTTTTTTTTTTTTTTTTTTTTTTTTT |
| 69Y | 0.979 | 0.99 | 1891 | YYYYYYYYYYYYYYYYYYYYYYYYYYYYYYYYYYYYYYYYYYYYYYYYYYYYYYYYYYYYYYYYYYY |
| 70W | 0.964 | 0.98 | 1556 | WWWWWWWW$WWWWWWWWWWWWWWWWWWWWWWWWWWWWWWWWWWWWWWWWWWWWWWWWWWWWWWWWWW |
| 71G | 0.99 | 0.995 | 2071 | GGGGGGGGGGGGGGGGGGGGGGGGGGGGGGGGGGGGGGGGGGGGGGGGGGGGGGGGGGGGGGGGGGG |
| 72L | 0.979 | 0.99 | 1892 | LLLLLLLLLLLLLLLLLLLLLLLLMLLLLLLLLLLLLLLLLLLLLLLLLLLLLLLLLLLLLLLLLLL |
| 73H | 0.908 | 0.94 | 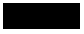 | HHHHHHHHHHHHHHHHHHHHQHNHHHHHHHHHHHHHHHHHNHHHHHHHHHHHHHHHHHHHHHHHHQH |
| 74T | 0.961 | 0.98 | 1335 | TTTTTTTTTTTTTTTTTTTTTTTTTTTTTTTTTTTTTTTTTTTTTTTTTTTSTTTTTTTTTTTTTTT |
| 75G | 0.99 | 0.995 | 2072 | GGGGGGGGGGGGGGGGGGGGGGGGGGGGGGGGGGGGGGGGGGGGGGGGGGGGGGGGGGGGGGGGGGG |
| 76E | 0.979 | 0.99 | 1893 | EEEEEEEEEEEEEEEEEEEEEEEEEEEEEEEEEEEEEEEEEEEEEEEEEEEEEEEEEEEEEEEEEEE |
| 77R | 0.951 | 0.97 | 1246 | RRRRRRRRRRRRRRRRKRRRRRRRRRRRRRRRRRRRRRRRRRRRRRRRKKKRRRRRRRRRRRRRRRR |
| 78D | 0.831 | 0.855 | 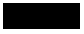 | NNNNDNDEDDNEDDEDDDEDDDDDDDDDDDEEDDEDDDDDDDEDDDDEEEEDDEDDDDDDDDDDDDD |
| 79W | 0.982 | 0.99 | 1909 | WWWWWWWWWWWWWWWWWWWWWWWWWWWWWWWWWWWWWWWWWWWWWWWWWWWWWWWWWWWWWWWWWWW |
| 80H | 0.99 | 0.995 | 2073 | HHHHHHHHHHHHHHHHHHHHHHHHHHHHHHHHHHHHHHHHHHHHHHHHHHHHHHHHHHHHHHHHHHH |
| 81L | 0.971 | 0.985 | 1676 | LLLLLLLLLLLLLLLLLLLLLLLLLLLLLFLMLLLLLLLLLLLLLLLLLLLLLLLLLLLLLLLLLLL |
| 82G | 0.979 | 0.99 | 1894 | GGGGGGGGGGGGGGGGGGGGGGGGGGGGGGGGGGGGGGGGGGGGGGGGGGGGGGGGGGGGGGGGGGG |
| 83Q | 0.956 | 0.975 | 1304 | QQQQQQQQQQQQQQQQQQQQQQQQQQQQQQNQQQQQQQQQQQQQQQQQQQHQQQQQQQQQHQQQQQQ |
| 84G | 0.979 | 0.99 | 1895 | GGGGGGGGGGGGGGGGGGGGGGGGGGGGGGEGGGGGGGGGGGGGGGGGGGGGGGGGGGGGGGGGGGG |
| 85V | 0.917 | 0.94 | 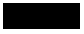 | IIVVVVVVVVVVVVVVVVVVVVVAVVVVVVAVVVAVVVVVVVVVVVVVVVVVVVVVVVVVVVVVVVV |
| 86S | 0.99 | 0.995 | 2074 | SSSSSSSSSSSSSSSSSSSSSSSSSSSSSSSSSSSSSSSSSSSSSSSSSSSSSSSSSSSSSSSSSSS |
| 87I | 0.943 | 0.965 | 1170 | IIIIIIIIIIIIIIIIIIIIIIIIIIIIIIIIIIIIIIIIIIIIIIIIIIIIIIIIIMVIVIIIIII |
| 88E | 0.971 | 0.985 | 1677 | EQEEEEEEEEEEEEEEEEEEEEDDEEEEEEEEEEEEEEEEEEEEEEEEEEEEEEEEEEEEEEEEEEE |
| 89W | 0.979 | 0.99 | 1896 | WWWWWWWWWWWWWWWWWWWWWWWWWWWWWWWWWWWWWWWWWWWWWWWWWWWWWWWWWWWWWWWWWWW |
| 90R | 0.964 | 0.98 | 1557 | RRRRRRRRRRRRRRRRRRRRRRRRRRRRRRRRRRRRRRRRRRKRRRRRRRRRRRRRRRRRRRRRRRR |
| 91K | 0.863 | 0.91 | 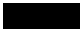 | KKQKKKKKKKKKKKKKKKKKKKKKKKKKKKKKKKKKKKTKKKKKKKKKKKKKKKKKKTKKKKKKKKK |
| tc,1 Table 3C-1 | | | | |
| 92K | 0.583 | 0.43 | 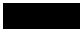 | #KRKKRKKRRKKRRGGEKRKKKKRKKRRRKRKRKKRKKKKRKKKKKRRRKRRKRGKKRRRKRRKKRK |
| 93R | 0.829 | 0.875 | 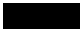 | #GRKRRRRRRRRTGRSRRRKSSGKRRRRRRRRRRRRRRRRRRRRRRRRRRRRRRRRRRRRRRRRRRR |
| 94Y | 1 | 1 | 2919 | #YYYYYYYYYYYYYYYYYYYYYYYYYYYYYYYYYYYYYYYYYYYYYYYYYYYYYYYYYYYYYYYYYY |
| 95S | 0.777 | 0.84 | 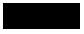 | #SRSSRSRSSSSRSSSASSSNNNKSSSSSSTSSSSSSRSSSSSSSSSSSSSSSSHSSSSSSSSSSSS |
| 96T | 0.979 | 0.99 | 1897 | #ITTSTTTTTTTTTTTTTTTTTTTTTTTTTTTTTTTTTTTTTTTTTTTTTTTTTTTTTTTTTTTTTT |
| 97Q | 0.99 | 0.995 | 2075 | #QQQQQQQQQQQQQQQQQQQQQQQQQQQQQQQQQQQQQQQQQQQQQQQQQQQQQQQQQQQQQQQQQQ |
| 98V | 0.951 | 0.97 | 1247 | #VVVVVVVVVVVVVVVVVVVVVVVVVVVVVVVVVVVVVVVVVVVVVVVVVVVVVVVVVVVVVVVVVV |
| 99D | 0.896 | 0.935 | 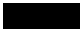 | #DDDDDDDDDDGDDDDTDGDDDDDDDDDDDDDDDDDDDDDDDDDDTDDDDDDDDDDDDDDTDDDDDE |
| 100P | 0.99 | 0.995 | 2076 | #PPPPPPPPPPPPPPPPPPPPPPPPPPPPPPPPPPPPPPPPPPPPPPPPPPPPPPPPPPPPPPPPPP |
| 101E | 0.514 | 0.045 | 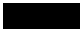 | #GDDNVGGGGDDEGEEDGDNDDNENDDGNGGGDGNDNGNGGNGGGDNNDGGGKGTNDNDNDDDDGED |
| 102L | 0.899 | 0.93 | 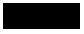 | #LLLLQLLLLLLLLQLILLLLLLLILLLLLLLLLLILLLILLLLLLLLLLILLLLLLLLIILLLLLL |
| 103A | 0.979 | 0.99 | 1898 | #AAAAAAAAAAAAAAAAAAAAAAAAAAAAAAAAAAAAAAAAAAAAAAAAAAAAAAAAAAAAAAAAAA |
| 104D | 0.99 | 0.995 | 2077 | #DDDDDDDDDDDDDDDDDDDDDDDDDDDDDDDDDDDDDDDDDDDDDDDDDDDDDDDDDDDDDDDDDD |
| 105Q | 0.86 | 0.905 | 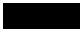 | #QQQQQQQQQQQQQQQQQQQQQQQQQQQQQQQQQQQQQQHQQQQQQQQQQQKQQQQKQQQQHQQQQQ |
| 106L | 1 | 1 | 2920 | #LLLLLLLLLLLLLLLLLLLLLLLLLLLLLLLLLLLLLLLLLLLLLLLLLLLLLLLLLLLLLLLLLL |
| 107I | 1 | 1 | 2921 | #IIIIIIIIIIIIIIIIIIIIIIIIIIIIIIIIIIIIIIIIIIIIIIIIIIIIIIIIIIIIIIIIII |
| 108H | 1 | 1 | 2922 | #HHHHHHHHHHHHHHHHHHHHHHHHHHHHHHHHHHHHHHHHHHHHHHHHHHHHHHHHHHHHHHHHHH |
| 109L | 0.819 | 0.88 | 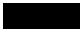 | #LVLTLLLLMLLIILILLLLLVLLLLLLLMLLLLLVMLLLLLLLLLLLLLMLVLLLLLLLLLLLLML |
| 110Y | 0.82 | 0.8 | 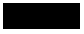 | #YYHYHYYFYYYHYYYYYHYYYYYYHYHYYYYYYYYHYYHYYHYYHYYYYYYHHHYYHYYYYYYYYY |
| 111Y | 0.969 | 0.985 | 1667 | #YYYYYYYFYYYYYYYYYYYYYYYYYYYYYYYYYYYYYYYYYYYYYYYYYYYYYYYYYYYYYYYYYY |
| 112F | 0.99 | 0.995 | 2078 | #FFFFFFFFFFFFFFFFFFFFFFFFFFFFFFFFFFFFFFFFFFFFFFFFFFFFFFFFFFFFFFFFFF |
| 113D | 0.954 | 0.975 | 1296 | #DDDDDDDDDDDDDADDDDDDDDDDDDDDDDDDDDDDDDDDDDDDDDDDDDDDDDDDDDDDDDDDDD |
| 114C | 0.99 | 0.995 | 2079 | #CCCCCCCCCCCCCCCCCCCCCCCCCCCCCCCCCCCCCCCCCCCCCCCCCCCCCCCCCCCCCCCCCC |
| 115F | 0.979 | 0.99 | 1899 | #FFFFFFFFFFFFFFFFFFFFFFFFFFFFFFFFFFFFFFFFFFFFFFFFFFFFFFFFFFFFFFFFFF |
| 116S | 0.942 | 0.965 | 1153 | #SSSSSSSSSSSSSSSASSSSSSSSSSSSSSSSSSSSSSSSSSSSSSSSSSSSTSSSSSSSSSSSSS |
| 117D | 0.861 | 0.15 | 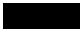 | #EEDEEEEEEEEDEDEDEEDEEEEEEEEEEEDEEEEEDEDEEDEEEEEEEEEDEEEEEEEEEEEEDE |
| 118S | 0.931 | 0.96 | 1027 | #SSSSSSSSSSSSSSSSSSSSTSSSSSSSSSSSSSSSSSSSSSSSSSSSSSSSSSSSSSSSSSSSSS |
| 119A | 1 | 1 | 2923 | #AAAAAAAAAAAAAAAAAAAAAAAAAAAAAAAAAAAAAAAAAAAAAAAAAAAAAAAAAAAAAAAAAA |
| 120I | 0.951 | 0.97 | 1248 | #IIIIIIIIIIIIIIMIIIIIIIIIIIIIIIIIIIIIIIIIIIIIIIIIIIIIIIIIIIIIIIIIII |
| 121R | 0.974 | 0.985 | 1775 | #RRRRRRRRRRRRRKRRRRRRRRRRRRRRRRRRRRRRRRRRRRRRRRRRRRRRRRRRRRRRRRRRRR |
| 122K | 0.526 | 0.255 | 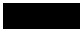 | #NHNKKNHHNNKANNHKNSKKNNGKSHNQNHNNNKKKNKNNKKNNNSKKKKSKNKQNNQHQSKKNKN |
| 123A | 0.919 | 0.95 | 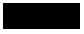 | #AAAATAVAAAAAAATAAAAAAAAAAAAAAAAAAAAAAAAAAAAAAAAAAAAAAAAAAAAAAAAAAA |
| 124L | 0.88 | 0.105 | 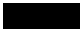 | #IIIIIIIIIILIILIIIIIIIIIIIIIIIIIIIIIIIIIIIIIIIILIIIILIIIIIIIILIIILI |
| 125L | 0.88 | 0.9 | 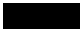 | #LLLLLLLLLLLLLLLLLLLLLLLLLLLLLVLLLLVVLLLLLVVLLLLLVVVLVVLLLLLLLLLVLL |
| 126G | 0.971 | 0.985 | 1678 | #GGGGGGGGGGGGGGGGGGGGGGGGGGGGGGGGGGGGGGGGGGGGGGGGGGGGGGGGGGGGGGGGGG |
| 127H | 0.659 | 0.655 | 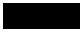 | #HHHHRRHHRNHHHHHRRHHHRRRHHHYHHRRHYHRHHHHRHRRHHHHDQHRHHHHHRHHHHHHRHR |
| 128I | 0.664 | 0.61 | 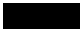 | #IIIIIRIIIRRRRIRIVRIIIIIIIIRRIIIRRILIIIRVIIIIIIIIIIIIRIRRVIIIIIRIII |
| 129V | 0.957 | 0.975 | 1318 | #VVVVVVVVVVVVVVVVVVVVVVVVVVVVVVVVVVVVGVVVVVVVVVVDVVVVVVVVVVVVVVVVVV |
| 130S | 0.808 | 0.855 | 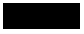 | #SINSSSSRSSSRRSSSSRSYRRSSSSSSSSSSSSNSSSSSSSRSSSSSSSSSSSSSSSSISNSSSS |
| 131P | 0.971 | 0.985 | 1679 | #PPPPPPPPPPPPPPPPPPPPPPPPPPPPPPPPPPPPPPPPPPPPPPPHPPPPPPPPPPPSPPPPPP |
| 132R | 0.738 | 0.605 | 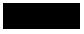 | #RKSSSSGRSRRSSSRSSRSSRRSSRRRRRRRSRSRRSRSSSSSRSRSSRSRRRRRSRRRSRSRSRS |
| 133C | 0.99 | 0.995 | 2080 | #CCCCCYCCCCCCCCCCCCCCCCCCCCCCCCCCCCCCCCCCCCCCCCCCCCCCCCCCCCCCCCCCCC |
| 134E | 0.841 | 0.86 | 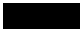 | #EEEEEEEEEEEEEEEDDEDEDDEDEDEEEEEEEEEEEEEEEEEEEDEEEEEEEEEEEEEEEEEEEE |
| 135Y | 0.925 | 0.955 | 949 | #YYYYYYYYHYYYYYYFYYYYYYYCYYYYYYYYYYYYYYYYYYYYYYYYYYYYYYYYYYYYYYYYYY |
| 136Q | 0.89 | 0.93 | 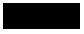 | #QQQQQQQQQQQQQPQRQQQQQQQQRPQRQQQQQQLQQQQQQQQQQQQQQQQQQQQQQQKQQQQQQQ |
| 137A | 0.951 | 0.97 | 1249 | #AAAAAAAAAAAAAAAAAAAATAAAAAAAAAAAAAAAAAAAAAAAAAAAAAAAAAAAAAAAAAAAAA |
| 138G | 1 | 1 | 2924 | #GGGGGGGGGGGGGGGGGGGGGGGGGGGGGGGGGGGGGGGGGGGGGGGGGGGGGGGGGGGGGGGGGG |
| 139H | 1 | 1 | 2925 | #HHHHHHHHHHHHHHHHHHHHHHHHHHHHHHHHHHHHHHHHHHHHHHHHHHHHHHHHHHHHHHHHHH |
| tc,1 Table 3C-2 | | | | |
| 92K | 0.583 | 0.43 | 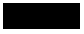 | ERRRQRKRRKRKKKEKKKKRRRRKKRKKKRRDKRKREKRRRREKEKDEKRRRKGERGKKRDREKKKR |
| 93R | 0.829 | 0.875 | 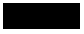 | RRRRKRRRRRRRRRRRRRRRRRRRRRRRRRRRRRRRGRRRRRRRKRRRRRRRKRRRRRGRRGRRRRR |
| 94Y | 1 | 1 | 2919 | YYYYYYYYYYYYYYYYYYYYYYYYYYYYYYYYYYYYYYYYYYYYYYYYYYYYYYYYYYYYYYYYYYY |
| 95S | 0.777 | 0.84 | 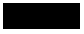 | SSSSSSSSSSSSSSSSSSSSSRCSSSSSNSSSSSSSSKSSSRNSKSSRSSSSSSSSSSNRRNSSSSS |
| 96T | 0.979 | 0.99 | 1897 | TTTTTTTTTTTTTTTTTTTTTTTTTTTTTTTTTTTTTTTTTTTTTTTTTTTTTTTTTTTTTTTTTTT |
| 97Q | 0.99 | 0.995 | 2075 | QQQQQQQQQQQQQQQQQQQQQQQQQQQQQQQQQQQQQHQQQQQQQQQQQQQQQQQQQQQQQQQQQQQ |
| 98V | 0.951 | 0.97 | 1247 | VVVVVVVVLVVVVVVVVVVVVVVVVVVVVVVVVVVVVVVVVVVVVVVVVVVVVVVVVVVVVVVVVVV |
| 99D | 0.896 | 0.935 | 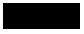 | DDDDDDDDDDDDDDDDDDEDDDDDDDDDDDDDDGDDDDDDDTDDDDDDDDDDDDDDDDDDDDADDDD |
| 100P | 0.99 | 0.995 | 2076 | PPPPPPPPPPPPPPPPPPPPPPPPPPPPPPPPPPPPPPPPPPPPPPPPPPPPPPPPPPPPPPPPPPP |
| 101E | 0.514 | 0.045 | 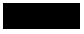 | NDGGGNGNNENGNNNGNNDNNEVDDDNGEDDANDNNNDNNNDDGDSADDNDDGTGDDGDGDDDGGSN |
| 102L | 0.899 | 0.93 | 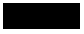 | LLLLLLLLLLLLLLLLLLLLLLLILLLLLLLLLLLLLLLLLLLILLLLLLLILLLLLLLLLLLLLLL |
| 103A | 0.979 | 0.99 | 1898 | AAAAAAAAAAAAAAAAAAAAAAAAAAAAAAAAAAAAAAAAAAAAAAAAAAAAAAAAAAAAAAAAAAA |
| 104D | 0.99 | 0.995 | 2077 | DDDDDDDDDDDDDDDDDDDDDDDDDDDDDDDDDDDDDDDDDDDDDDDDDDDDDDDDDDDDDDDDDDD |
| 105Q | 0.86 | 0.905 | 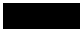 | QQQQQQQQQQHQQQQQQQRRQQQQQQQQQQQQQQQQQQQQQQQQQQQRQHQQQQQQQQQQQQQQQQQ |
| 106L | 1 | 1 | 2920 | LLLLLLLLLLLLLLLLLLLLLLLLLLLLLLLLLLLLLLLLLLLLLLLLLLLLLLLLLLLLLLLLLLL |
| 107I | 1 | 1 | 2921 | IIIIIIIIIIIIIIIIIIIIIIIIIIIIIIIIIIIIIIIIIIIIIIIIIIIIIIIIIIIIIIIIIII |
| 108H | 1 | 1 | 2922 | HHHHHHHHHHHHHHHHHHHHHHHHHHHHHHHHHHHHHHHHHHHHHHHHHHHHHHHHHHHHHHHHHHH |
| 109L | 0.819 | 0.88 | 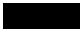 | LLMLVLLLLLLLLLLLLLLLLLLLLLLLLLLLLLLLLLLLMLLLLLLLLLLLLLLLLTLQLLLLLLL |
| 110Y | 0.82 | 0.8 | 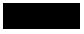 | HHYHYHYYHYYYYYYYYYHYYYYYYYYYYYYYYYYYYYYYYYHYYYYYHYYYYYYYYYYYYYYHYYY |
| 111Y | 0.969 | 0.985 | 1667 | YYYYYYYYYYYYYYYYYYYYYYYYYYYYYYYYYYYYYYYYYYYYYYYYYYYYYYYYYYYYYYYYYYY |
| 112F | 0.99 | 0.995 | 2078 | FFFFFFFFFFFFFFFFFFFFFFFFFFFFFFFFFFFFFFFFFFFFFFFFFFFFFFFFFFFFFFFFFFF |
| 113D | 0.954 | 0.975 | 1296 | DDDDDDDDDDDDDDNDDDDDDDDDDDDDDDDDDDDDDDDDDDDDDDDDDDDDDDDDDDDDDDDDDDD |
| 114C | 0.99 | 0.995 | 2079 | CCCCCCCCCCCCCCCCCCCCCCCCCCCCCCCCCCCCCCCCCCCCCCCCCCCCCCCCCCCCCCCCCCC |
| 115F | 0.979 | 0.99 | 1899 | FFFFFFFFFFFFFFFFFFFFFFFFFFFFFFFFFFFFFFFFFFFFFFFFFFFFFFFFFFFFFFFFFFF |
| 116S | 0.942 | 0.965 | 1153 | SSSTSSSSSSSSSSSSSSSSSSSSSSSSSSSSSSSSSSSSSSSSSSSSSSSSSSSSSSSSSSSSSSS |
| 117D | 0.861 | 0.15 | 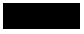 | DEEEDEEEEDEEEEEEDEEEEEEEEEEEEEEEEEEEEEEEEEEEEEEEDEEEDEEEEEEEEEEEDEE |
| 118S | 0.931 | 0.96 | 1027 | SSSSSSSSSSSSSSSSSSSSSSSFSSSFSSSFSSSSSSSSSSSSSSSSSSSSSSSSSSSSSTSSSSF |
| 119A | 1 | 1 | 2923 | AAAAAAAAAAAAAAAAAAAAAAAAAAAAAAAAAAAAAAAAAAAAAAAAAAAAAAAAAAAAAAAAAAA |
| 120I | 0.951 | 0.97 | 1248 | IIIIIIIIIIIIIIIIVIVIIIIIIIIIIVIIIIIIIIIIIIIIIIIIIIIIIIIIIIIIIIIIIII |
| 121R | 0.974 | 0.985 | 1775 | RRRRRRRRRRRRRRRRRRRRRRRRRRRRRRRRRRRRRRRRRRRRRRRRRRRRRRRRRRRRRRRRRRR |
| 122K | 0.526 | 0.255 | 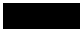 | NSKNQNNNNKNNNNNNKNNGKGGNNNSNNHQNNGNSKNNNNNNNNSNKSNQDNNQNNNKNKKKKNNK |
| 123A | 0.919 | 0.95 | 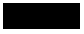 | AAAAAAAAAAAAAAAAAAAAAAAATAAAAAAAAAAAAAAAATAAAAAAAAAAAAAAAAAAAAAAAAA |
| 124L | 0.88 | 0.105 | 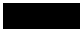 | IIIILIIIILIIIIIIIIIIIIIIIIIIIIIIILIIIIIIIIIIIIIIIIIIIIIIIIILLLIIIIL |
| 125L | 0.88 | 0.9 | 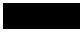 | LLLVLLLLLLLLLLLLLLLLLLLLLLLLLLLLLLLLILLLLLLLLVLLLLLLLLLLLLVLLLLVLLL |
| 126G | 0.971 | 0.985 | 1678 | GGGGGGGGGGGGGGGGGGGGGGGGGGGGGGGGGGGGGGGGGGGGGGGGGGGGGGGGGGGGGGGGGGG |
| 127H | 0.659 | 0.655 | 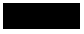 | HHHHHRRRHHYHHHHHHHRHHHRRRHHHHRHRHHHHHHHHHHRHDHRHRHHRHHHHRNRRYHHRHHK |
| 128I | 0.664 | 0.61 | 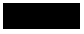 | IRIRIIIIIIRIIIRIIILIRIILLRIIRLRIIIIIIRIIIRLIRIIILIIIIIKIIILTRRILIII |
| 129V | 0.957 | 0.975 | 1318 | VVVVVVGVVVVVVVVVVVVVVVVVVVVVVVVGGVVVVVVVVVVVVVVVVVVVVVVVVVVVVVVVVVV |
| 130S | 0.808 | 0.855 | 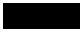 | SSSSSSSSSSSSSQSISSSSSSSSSSSISSRSSSSSSSSSSSSSSSSSRSSSSSSSSSSSSSSSSSS |
| 131P | 0.971 | 0.985 | 1679 | PPPPPPPPPPPPPSPPPPPPPPPPPPPPPPPPPPPPPPPPPPPPPPPPPPPPPPPPPPPPPPPPPPP |
| 132R | 0.738 | 0.605 | 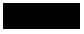 | RSSRRRSRRRRSSRRSRRRRSSSSRRRSSRRRRRRRRRRSSRQSRRRSRRSRRRRSSRRSRSSRRSS |
| 133C | 0.99 | 0.995 | 2080 | CCCCCCCCCCCCCCCCCCCCCCCCCCCCCCCCCCCCCCCCCCCCCCCCCCCCCCCCCCCCCCCCCCC |
| 134E | 0.841 | 0.86 | 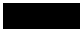 | EEEEEEEEEEEDDEEEEEEDEEEEEEDEEEEEEEEEEEEEEEEDEEEEEEEEDEEEEEEKDEEEEEE |
| 135Y | 0.925 | 0.955 | 949 | YYYYYYYYYYYYYYYYYYYYYYYYYYYYYYYYYYYYYYYYYYYYYYYYYYYYYYYYYYYYYYYYYYY |
| 136Q | 0.89 | 0.93 | 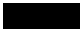 | QQQQQQRQQQQQQQQQQQQQQRQQQQQQQQQQQQQQQQQQQQLQQQQQQQQQQQQQQPQQPQQQQQQ |
| 137A | 0.951 | 0.97 | 1249 | AAAAAAAAAAAAAAAAAAAAAAAAAAAAAAAAAAAAAAAAAATAAAAAAAAAAAAAAAAATAAAAAA |
| 138G | 1 | 1 | 2924 | GGGGGGGGGGGGGGGGGGGGGGGGGGGGGGGGGGGGGGGGGGGGGGGGGGGGGGGGGGGGGGGGGGG |
| 139H | 1 | 1 | 2925 | HHHHHHHHHHHHHHHHHHHHHHHHHHHHHHHHHHHHHHHHHHHHHHHHHHHHHHHHHHHHHHHHHHH |
| tc,1 Table 3C-3 | | | | |
| 92K | 0.583 | 0.43 | 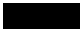 | KKEERKKERKRKKRERRKKERNSNKRRRRRKEKRKRRGRRRTRRERKRRERKRKKKKRKKKQKQRKK |
| 93R | 0.829 | 0.875 | 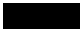 | RKRRRRRRRGRRRRRRRRRRKRRRRRRRRKRRRRRRRRRRRRRRRRRKRRSRRKRRRRRRRSRRRRR |
| 94Y | 1 | 1 | 2919 | YYYYYYYYYYYYYYYYYYYYYYYYYYYYYYYYYYYYYYYYYYYYYYYYYYYYYYYYYYYYYYYYYYY |
| 95S | 0.777 | 0.84 | 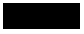 | SSNSNSRSSSSNSSSSSSSISSSSSSSSSSSSSSSSSSSSSSSSSNSSSSSSSSSSSSNSSRSSSSS |
| 96T | 0.979 | 0.99 | 1897 | TTTTTTTTTTTTTTTTTTTTTTTTTTTTTTTTTTTTTTTTTTTTTTTTTTTTTTTTTTTTTTTTTTT |
| 97Q | 0.99 | 0.995 | 2075 | QQQQQQQQQQQQQQQQQQQQQQQQQQQQQQQQQQQQQQQQQQQQQQQQQQQQQQQQQQQQQQQQQQQ |
| 98V | 0.951 | 0.97 | 1247 | VVVVVVVVVIVVVVIVVVIVIVVVVVVVVVIVVVVVVVVVVVVVVVVVVVVVVVVVVVVVVVVVVVV |
| 99D | 0.896 | 0.935 | 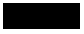 | DDDDDDDDDDDDDDDDDDDTDDDDDDDDDDDDDDDDDDDDDDDDDTDDDDDDDDDDDDDDDTDDDDD |
| 100P | 0.99 | 0.995 | 2076 | PPPPPPPPPPPPPPPPPPPPPPPPPPPPSPPPPPPPPPPPPPPPPPPPPPPPPPPPPPPPPPPPPPP |
| 101E | 0.514 | 0.045 | 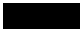 | NGDGDDGDGGNDGDDDDDGDDDDDDNNNNDNTDDGDGDNIHNDGDDNNDNEGDGDDGDDDDDGDDDG |
| 102L | 0.899 | 0.93 | 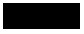 | LLLLLLLLLLLLLQLLLLLLLLLLLLLLLLLLLLLLLLLLLLLLLLTLLLLLLLLLLLLLLLLLLLL |
| 103A | 0.979 | 0.99 | 1898 | AAAAAAAAAAAAAAAAAAAAAAAAAAAAAAAAGAAAAAAAAAAAAAAAAAAAAAAAAAAAAAAAAAA |
| 104D | 0.99 | 0.995 | 2077 | DDDDDDDDDDDDDDDDDDDDDDDDDDDDEDDDDDDDDDDDDDDDDDDDDDDDDDDDDDDDDDDDDDD |
| 105Q | 0.86 | 0.905 | 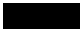 | QQQQQQQQQQQKQQQQRRRQQQQQQQQQEQQQQQQQQQQHQHHQQQQQQQQQQQHQQQQQQQRQQQQ |
| 106L | 1 | 1 | 2920 | LLLLLLLLLLLLLLLLLLLLLLLLLLLLLLLLLLLLLLLLLLLLLLLLLLLLLLLLLLLLLLLLLLL |
| 107I | 1 | 1 | 2921 | IIIIIIIIIIIIIIIIIIIIIIIIIIIIIIIIIIIIIIIIIIIIIIIIIIIIIIIIIIIIIIIIIII |
| 108H | 1 | 1 | 2922 | HHHHHHHHHHHHHHHHHHHHHHHHHHHHHHHHHHHHHHHHHHHHHHHHHHHHHHHHHHHHHHHHHHH |
| 109L | 0.819 | 0.88 | 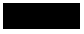 | LMLLLLLLLLLLLLLRLLLLLLLLLLLLLQLLLLMLLLLLLLLLLLLLLLLLLLLLILLLLLLLLLM |
| 110Y | 0.82 | 0.8 | 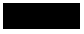 | YYYYYYYYHYYHYYHYHHYYYYHYYYSYYYYYYYYHYHYYYYYYHYHYHHYYYHHHYYYYYYYYYYY |
| 111Y | 0.969 | 0.985 | 1667 | YYYYYYYYYYYYYYYYYYYYYYYYYYSYYYYYYYYYYYYYYYYYYYYYYYYYYYYYYYYYYYYHYYY |
| 112F | 0.99 | 0.995 | 2078 | FFFFFFFFFFFFFFFFFFFFFFFFFFFFLFFFFFFFFFFFFFFFFFFFFFFFFFFFFFFFFFFFFFF |
| 113D | 0.954 | 0.975 | 1296 | DDDDDDDDDDDDDDDDDDNDDDDDDYDDDDDDDDDDDDDDDDDDDDDDDDDDDDDDDDDDDNDDDDD |
| 114C | 0.99 | 0.995 | 2079 | CCCCCCCCRCCCCCCCCCCCCCCCCCCCCCCCCCCCCCCCCCCCCCCCCCCCCCCCCCCCCCCCCCC |
| 115F | 0.979 | 0.99 | 1899 | FFSFFFFFFFFFFFFFFFFFFFFFFFFFFFFFFFFFFFFLFFFFFFFFFFFFFFFFFFFFFFFFFFF |
| 116S | 0.942 | 0.965 | 1153 | SSSSSSSSSSSSSSTSSSSSSSSSSSSSSSSSSSSSSSSASSSASSSSSSASSSSSSSSSSSSSSSS |
| 117D | 0.861 | 0.15 | 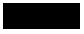 | EDEEEEEEDEEDEEDDEEEEEEEEEEEEEEEEEEEDEEEDEEEEDEEEEDDEEEDEEEEEEEDEEEE |
| 118S | 0.931 | 0.96 | 1027 | ASSSSSSSSSSSSSSSSSSSSSSSSSSSSSSSSSSSSSSSSSSSSASSSSSSSSSSSSSSSSSSSSS |
| 119A | 1 | 1 | 2923 | AAAAAAAAAAAAAAAAAAAAAAAAAAAAAAAAAAAAAAAAAAAAAAAAAAAAAAAAAAAAAAAAAAA |
| 120I | 0.951 | 0.97 | 1248 | IIIIIIIIIIIIIIIIIIIIIIIIIIIIIIIIIIIIIIIIIIVIIIIIVIIIIIIIIIIIIIIIIII |
| 121R | 0.974 | 0.985 | 1775 | RRRRRRRRRRRRRRRRRRRRRRKRRRRRRRRRRRRRRRRRRRRRRRRRRRRRRKRRRRRRRRRRRRR |
| 122K | 0.526 | 0.255 | 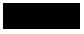 | KNKNQHNKKNNHNKNNQDQNNNKKRNNNNNKNNQKHQNNKSKSSNSNKENKNKNKNNNNKNQKNNNN |
| 123A | 0.919 | 0.95 | 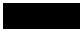 | AAAAAAAAAAAAAAAAAAAAAAAAAAAAAAAATAAAAAAAAAAAAAAATAAAPAATAAAAAASAAAA |
| 124L | 0.88 | 0.105 | 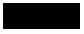 | IIILIIIIIIIIIIIILLIIILLIIIIIIILIIIILIIIIIIIIIIIIIILISIIIIIIIIIIIIII |
| 125L | 0.88 | 0.9 | 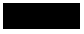 | LLLLLLLLLLLLLVLLLLLLLLLLLLLLLLLLLLTLLLVLLLLLLLLLLLLLLLLLLLLLLVLLLLL |
| 126G | 0.971 | 0.985 | 1678 | GGGGGGGGGGGGGGGGGGGGGGGGGGGGGGGGGGGGGGGGGGGGGGGGGGGGGGGGGGGGGGGGGGG |
| 127H | 0.659 | 0.655 | 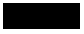 | QRHHHHHHHHRHHCRHHHYHHLLLHRRRRQHHCHHHHHRHHHHHHHRRHHHHHYHRYHHYHRHHHHR |
| 128I | 0.664 | 0.61 | 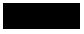 | IIRRRRRSIRRRVRLIRRRIVRRRRIIIIIRRIRILILLILIIIIIIIRIIRIRRIRIIRIIILRII |
| 129V | 0.957 | 0.975 | 1318 | VVVVVVVVVVVVVVVVVVVVVVVVVVVVVVVVVVVVVVVVVVVVVVVVVVVVVVVVVVVVVVVVVVV |
| 130S | 0.808 | 0.855 | 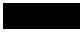 | SSSRSSRSSRSRSSSSSSSSSSSSSRRRRSSSSSSSSSSSSSSSSSTSSRSSSSSSSSSSSSSISSS |
| 131P | 0.971 | 0.985 | 1679 | PPPPPPPPPPPPPPPPPPPPPPPPPPPPPPPPPPPPPPPPPPPPPPPPPPPPPPPPPPPPPPPPPPP |
| 132R | 0.738 | 0.605 | 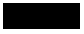 | SRRRRSSRSSRKRRRSRRRRRRRRSSSSSRRRRRSRRRRRRRSSRSSRRRSRRIRRRRRRRRSRRRR |
| 133C | 0.99 | 0.995 | 2080 | CCCCCCCCCCCCCCCCCCCCCCCCCCCCCCCCCCCCCCCCCCCCCCCCCCCCCCCCCCCCCCCCCCC |
| 134E | 0.841 | 0.86 | 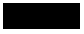 | DEEEEEEEKEEEEEEEEEEDEEEEEDDDDENEEEEEEEEEDEEEEEEDEEAEEEEEEEEEEEEEEED |
| 135Y | 0.925 | 0.955 | 949 | YYYYYYYYYYYYYYYYYYYYYYYYYYYYFYYYYYYYYYSYYYYYYYYYYYYYYYFYYYYYYYYYYYY |
| 136Q | 0.89 | 0.93 | 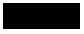 | QQQQQQQQQQRQQQQQQQQQQQQQQQQQQQQQQQQQQQQQQRQQQQQQQQQQQQQQQQQQQQQQQQQ |
| 137A | 0.951 | 0.97 | 1249 | AAAAAAAAAAAAAAAAAAAAAAAADAAAAAAAAAAAAATAAAAAAAAAAAAAAAAAAAAAATAAAAA |
| 138G | 1 | 1 | 2924 | GGGGGGGGGGGGGGGGGGGGGGGGGGGGGGGGGGGGGGGGGGGGGGGGGGGGGGGGGGGGGGGGGGG |
| 139H | 1 | 1 | 2925 | HHHHHHHHHHHHHHHHHHHHHHHHHHHHHHHHHHHHHHHHHHHHHHHHHHHHHHHHHHHHHHHHHHH |
| tc,1 Table 3D-1 | | | | |
| 140N | 0.872 | 0.91 | 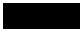 | #NNNNNNSNNNNNNNNNNNNNNNNNNNNNNNNNNNNNNNNNNNNNNNNNNNNNNNNNSNNKNNNINN |
| 141K | 0.964 | 0.98 | 1558 | #KKKKKKKTKKKKKKKKKKKKKKKKKQKKKKKKKKKKKQKKKKKKKKKKKKKKKKKKKKKKKKKKKK |
| 142V | 0.974 | 0.985 | 1776 | #VVVVVVVVVVVVVVVVVVVVIVVVVVVVVVVVVVVVVVVVVVVVVVVVVVVVVVVVVVVVVVVVVV |
| 143G | 0.982 | 0.99 | 1910 | #GGGGGGGGGGGGGGGGGGGGGGGGGGGGGGGGGGGGGGGGGGGGGGGGGGGGGGGGGGGGGGGGGG |
| 144S | 0.933 | 0.96 | 1035 | #SSSSSSSSSSSSSSSSSSSSSSSSSSSSSSSTSSSSSSSSSSSTSSSSSSSSSSSSSSSSSSSSSS |
| 145L | 0.99 | 0.995 | 2081 | #LLLLLLLLLLLLLLLLLLLLLLLLLLLLLLLLLLLLLLLLLLLLLQLLLLLLLLLLLLLLLLLLLL |
| 146Q | 0.979 | 0.99 | 1900 | #QQQQQQQPQQQQQQQQQQQQQQQQQQQQQQQQQQQQQQQQQQQQQQQQQQQQEQQQQQQQQQQQQQ |
| 147Y | 1 | 1 | 2926 | #YYYYYYYYYYYYYYYYYYYYYYYYYYYYYYYYYYYYYYYYYYYYYYYYYYYYYYYYYYYYYYYYYY |
| 148L | 1 | 1 | 2927 | #LLLLLLLLLLLLLLLLLLLLLLLLLLLLLLLLLLLLLLLLLLLLLLLLLLLLLLLLLLLLLLLLLL |
| 149A | 1 | 1 | 2928 | #AAAAAAAAAAAAAAAAAAAAAAAAAAAAAAAAAAAAAAAAAAAAAAAAAAAAAAAAAAAAAAAAAA |
| 150L | 1 | 1 | 2929 | #LLLLLLLLLLLLLLLLLLLLLLLLLLLLLLLLLLLLLLLLLLLLLLLLLLLLLLLLLLLLLLLLLL |
| 151A | 0.712 | 0.665 | 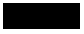 | #AVATRAAAAAASTAAATTATTTTATAATTTATAATAATAAAATAAAATAAATTTATAAAAAAAAAA |
| 152A | 0.937 | 0.96 | 1124 | #AAATAAAAAAAAAAAAAAAAAAAAAAAAAAAAAAAAAAAAAAAAAAAAAAAAAAAAAAAAAAAAAA |
| 153L | 0.99 | 0.995 | 2082 | #LLLLLLLLLLLLLLLLLLLLLLLLLLLLLLLLLLLLLLLLLLLLLLLLLLLLLLLLLLLLLLLLLL |
| 154I | 0.677 | 0.675 | 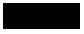 | #IIVIIVIVIIIIITIVTIIIIIIIVIVIIITIIVIIIIIIVIILIIIIIIIIIIIVIVVITTVIII |
| 155T | 0.742 | 0.775 | 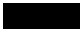 | #TKSKTTTTTTTTTTPTTTTTTTTTTTTTAATTPTTTATTRKTTTTTTKTKTTAATTTTTKTAKTTT |
| 156P | 0.939 | 0.965 | 1134 | #PPPPPPPPPPPPPPPPPPPPPPPPPPPPPPPPSPPPPPPPPPPPPPPPPPPPPPPPPPPPPPPPPP |
| 157K | 0.826 | 0.865 | 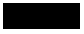 | #KKKKKQKERKKKKKKKKKKKKKKKKKKKKKKKKKKKRRKKKKKKKKRKKKKKRRKKKKKKTKKKKK |
| 158K | 0.8 | 0.82 | 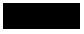 | #KKKKKQKEQKKKKKKKNKRKKKKKKKRKKKKRKKKKKQKQKKKRKKRKKRKKKKRKKKKKKKKKKK |
| 159I | 0.69 | 0.675 | 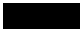 | #TIIIIIIITIIIIIITRTVIIRIIIIRIIIIIRTIRTTITIIIIRIIIIIIIIITIITTKIIITRR |
| 160K | 0.904 | 0.935 | 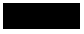 | #KKKKKQKEKKKKKKKKKKKKKKKKKKKKKKKKKKKKKKKKKKKKKKKKKKKKKKKKKKKRKKKKRR |
| 161P | 1 | 1 | 2930 | #PPPPPPPPPPPPPPPPPPPPPPPPPPPPPPPPPPPPPPPPPPPPPPPPPPPPPPPPPPPPPPPPPP |
| 162P | 1 | 1 | 2931 | #PPPPPPPPPPPPPPPPPPPPPPPPPPPPPPPPPPPPPPPPPPPPPPPPPPPPPPPPPPPPPPPPPP |
| 163L | 0.941 | 0.965 | 1144 | #LLLLLLLLLLLLLLLLLLFLLLLLLFLLLLLLLLLLLLLLLLLLVLLLLLLLLLFLLLLLLLLLLL |
| 164P | 1 | 1 | 2932 | #PPPPPPPPPPPPPPPPPPPPPPPPPPPPPPPPPPPPPPPPPPPPPPPPPPPPPPPPPPPPPPPPPP |
| 165S | 0.979 | 0.99 | 1901 | #SSSSSSSSSSSSSSSSSSSSSSSSSSSSSSSSSSSSSSSSSSSSSSSSSSSSSSSSSSSSSSSSSS |
| 166V | 0.945 | 0.965 | 1185 | #VVIVVVVVVVVVVVVLVVVVVVVVVVVVVVVVVVVVVVVVVVVVVVVVVVVVVVVVVVVVVVVVVV |
| 167T | 0.524 | 0.4 | 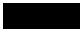 | #TKKVTVTATATAATTQTRARRRKTKKATARTRTTRATATTRAATRTRKTTTTTTAAKAAKTKKRAT |
| 168K | 0.969 | 0.985 | 1668 | #KKKKKKKTKKKKKKKIKKKKKKKKKKKKKKKKKKKKKKKKKKKKKKKKKKKKKKRKKKKKKKKKKK |
| 169L | 0.99 | 0.995 | 2083 | #LLLLLLLLLLLLLLLLLLLLLLLLLLLLLLLLLLLLLLLLLLLLLLLLLLLLLLLLLLLLLLLLLL |
| 170T | 0.971 | 0.985 | 1680 | #TTTTTTTTTTTTTTTTTTTTTTTTTTTTTTTTTTTTTTTTTTTTTTTTTTTTTTTTTTTTTTTTTT |
| 171E | 1 | 1 | 2933 | #EEEEEEEEEEEEEEEEEEEEEEEEEEEEEEEEEEEEEEEEEEEEEEEEEEEEEEEEEEEEEEEEEE |
| 172D | 1 | 1 | 2934 | #DDDDDDDDDDDDDDDDDDDDDDDDDDDDDDDDDDDDDDDDDDDDDDDDDDDDDDDDDDDDDDDDDD |
| 173R | 1 | 1 | 2935 | #RRRRRRRRRRRRRRRRRRRRRRRRRRRRRRRRRRRRRRRRRRRRRRRRRRRRRRRRRRRRRRRRRR |
| 173W | 0.982 | 0.99 | 1911 | #WWWWWW$$WWWWWWWWWWWWWWWWWWWWWWWWWWWWWWWWWWWWWWWWWWWWWWWWWWWWWWWWWW |
| 175N | 1 | 1 | 2936 | #NNNNNNNNNNNNNNNNNNNNNNNNNNNNNNNNNNNNNNNNNNNNNNNNNNNNNNNNNNNNNNNNNN |
| 176K | 0.93 | 0.955 | 1017 | #KEKKKKKKKKKKKKKKKKKKKKKEKKKKKKKKKKKKKKKKKKKKNEKKKKKKKKKKKKKKKKKKKK |
| 177P | 0.947 | 0.97 | 1218 | #PPPPPPPPPPPPPPPPPPPPPPPPPPPPPPPPPPPPPPPPPPPPPPPSPPPPPPPPPPPPPPPPPP |
| 178Q | 0.89 | 0.91 | 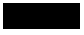 | #QQQQQQQQRQQQQQQQQQRQQQRQQRQQQQQQQQQQQQQQQQQQQQQRQRQQQQQQQQQQQQQQQQ |
| 179K | 0.902 | 0.93 | 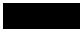 | #KKKKKKKKKKKKKKKKRKKKKKKRKKKKRKKKRRKKRKKKKKKKKKKKKKKKKKRKKKKKKKKRKK |
| 180T | 0.95 | 0.965 | 1239 | #TTTTTTTTTTITTITTTTTTTTTTTTTTTTTTTTTTTTTTTTTTTTTTTTTTTTTTTTTTTTTTTT |
| 181K | 0.947 | 0.97 | 1219 | #KKKKKKKKKKKKKKKKKKKRRKKKKKKKKKKKKKKKKKKKKKKKKKKKKKKKKKKKKKKKKKKKKK |
| 182G | 0.99 | 0.995 | 2084 | #GGGGGGGGGGGGGGGGGGGGGGGGGGGGGGGGGGGGGGGGGGGGGGGGGGGGGGGGGGGGGGGGGG |
| 183H | 0.935 | 0.955 | 1058 | #HRHHHHHHHHHHHHHHHHHHHRHHHHHHHHHHHHHHHHRHHHHHHHHHHHHHHHHHHHHHHHHHHH |
| 184R | 0.894 | 0.91 | 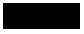 | #RRRRRRRRRRRRRRRRRRRRRRRRRRRRRRRRRRRRRKRRRRRRRKRRRRRRRRRRRRRRRRRRRR |
| 185G | 0.883 | 0.895 | 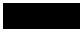 | #EGGEEGREGGGGGGGEGGGGGGGGGGGEGEEEGEGGGGGGGGGGGGGGGGGGGGGGGGGGGGGGGG |
| 186S | 0.895 | 0.925 | 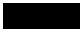 | #SSSSSSSNSSSSSRSSSSSSSSSSSSSSSSSSSNNTSSSSSSSSSSSSSSSSSSSSSSSSSSSSSS |
| 187H | 0.964 | 0.98 | 1559 | #HHHHHHHHHHHHHHHHHHHHHHHHHHHHHHHHHHHHHHHHHHHHHHHHHHHHHHHHHHHHHHHHHH |
| tc,1 Table 3D-2 | | | | |
| 140N | 0.872 | 0.91 | 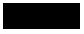 | NNNNNNNSNNSNNNNNSNSNNNNNNSNNNSNNNNNNNNNNNNNNNNNNNNNRNNNNNNNNNNNNNNT |
| 141K | 0.964 | 0.98 | 1558 | KKKKKKKKKKKKKKKKKKKKKKKKKKKKKKKKKKKKKKKKKKKKKKKKKKKKKKKKKKKKKKKKKKK |
| 142V | 0.974 | 0.985 | 1776 | VVVVVIVVVVVVVVVVVVVVVVVVVVVVVVVVVVVVVVVVVVVVVVVVVVVVVVVVVVVVVVVVVVV |
| 143G | 0.982 | 0.99 | 1910 | GGGGGGGGGGGGGGGGGGGGGGGGGGGGGGGGGGGGGGGGGGGGGGGGGGGGGGGGGGGGRGGGGGR |
| 144S | 0.933 | 0.96 | 1035 | SSSSSSSSSSSSSSSSSSSSSSSSSSSSSSSSSSSSSSSSSSSSSSSSSSSSSSSSSSSSSSSSSSF |
| 145L | 0.99 | 0.995 | 2081 | LLLLLLLLLLLLLLLLLLLLLLLLLLLLLLLLLLLLLLLLLLLLLLLLLLLLLLLLLLLLLLLLLLL |
| 146Q | 0.979 | 0.99 | 1900 | QQQQQQQQQQQQQQQQQQQQQQQQQQQQQQQQQQQQQQQQQQQQQQQQQQQQQQQQQQQQQQQQQQQ |
| 147Y | 1 | 1 | 2926 | YYYYYYYYYYYYYYYYYYYYYYYYYYYYYYYYYYYYYYYYYYYYYYYYYYYYYYYYYYYYYYYYYYY |
| 148L | 1 | 1 | 2927 | LLLLLLLLLLLLLLLLLLLLLLLLLLLLLLLLLLLLLLLLLLLLLLLLLLLLLLLLLLLLLLLLLLL |
| 149A | 1 | 1 | 2928 | AAAAAAAAAAAAAAAAAAAAAAAAAAAAAAAAAAAAAAAAAAAAAAAAAAAAAAAAAAAAAAAAAAA |
| 150L | 1 | 1 | 2929 | LLLLLLLLLLLLLLLLLLLLLLLLLLLLLLLLLLLLLLLLLLLLLLLLLLLLLLLLLLLLLLLLLLL |
| 151A | 0.712 | 0.665 | 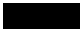 | TAATAAAATAAVAAVAAATAAAATAVAAVTAATAAAAAAAAATAALAATKAAATAAAATTTATATAA |
| 152A | 0.937 | 0.96 | 1124 | AAAAAAAAAAAAAVAVAAAAAAAAAAAVAAAAAAAAAAAAAAAAAAAAAAAAAAAAAAAAAAAAAAA |
| 153L | 0.99 | 0.995 | 2082 | LLLLLLLLLLLLLLLLLLLLLLLLLLLLLLLLLLLLLLLLLLLLLLLLLLLLLLLLLLLLLLLLLLL |
| 154I | 0.677 | 0.675 | 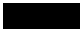 | VIVITIITVIIIIIIIIIIVIIIIIIVIITIRTIIIIIILIIVIIVRIIVIIIVRIIIIIIIIIIRV |
| 155T | 0.742 | 0.775 | 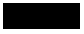 | TTTKTTTTRTTTTTTTTATTTTTKTTTTTTTTTTTTTTTTTTTTKTTKKTKTTTTTTKATTTTTTTT |
| 156P | 0.939 | 0.965 | 1134 | PPPPPPPPPPPPPPPPPPQPPPPPPPPPPQPPPPPPPPPPPPPPPPPPPPPPPPPPPPPPPPPPPPP |
| 157K | 0.826 | 0.865 | 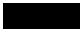 | KKKKKKKKKKKKKKKKKKKKKTTKKKRKKRERKTKKKKRKKKKKKKRKKRKKKKKTKKKKKKKKKKK |
| 158K | 0.8 | 0.82 | 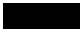 | KKQKKKKKKKKKKKKKKKRRKKKRKKKKKKKKKKKKKKKKKKKKKKKKRKKKKKKKKKKKKKKKKKK |
| 159I | 0.69 | 0.675 | 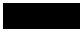 | IITIRIIIIIIIIRIIRIIRIIRIIIRITIITIIKITITIRTIIIRTRITITIRIRIIIIKIRITII |
| 160K | 0.904 | 0.935 | 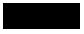 | KKRKKKKKKKKKKKKKKKKKKKKRRKKKKKKRKKKKKKKKKKRKKKKKTKKKKKKKKKKKKKKKKKK |
| 161P | 1 | 1 | 2930 | PPPPPPPPPPPPPPPPPPPPPPPPPPPPPPPPPPPPPPPPPPPPPPPPPPPPPPPPPPPPPPPPPPP |
| 162P | 1 | 1 | 2931 | PPPPPPPPPPPPPPPPPPPPPPPPPPPPPPPPPPPPPPPPPPPPPPPPPPPPPPPPPPPPPPPPPPP |
| 163L | 0.941 | 0.965 | 1144 | LLLLFLLLLLLLLLLLLLLLLLLLLLLLLLLLLLLLLLLLLLLLLLLLLLLLLLLLLLLLLLLLLLL |
| 164P | 1 | 1 | 2932 | PPPPPPPPPPPPPPPPPPPPPPPPPPPPPPPPPPPPPPPPPPPPPPPPPPPPPPPPPPPPPPPPPPP |
| 165S | 0.979 | 0.99 | 1901 | SSSSSSSISSSSSSSSSSSSSSSSSSSSSSSSSSSSSSSSSSSSSSSSSSSSSSSSSSSSSSSSSSS |
| 166V | 0.945 | 0.965 | 1185 | VIVVIVVVVVVVVVIVVVVVVVVVVVVVVIVVVVVVVVVVVVVVVVVVVVVVVVVVVVVVVVVIVVV |
| 167T | 0.524 | 0.4 | 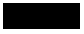 | KKTTKTATTTVTTXTTATKATTTRKTATTKRATTTTTKTTTTRAKATTKTKTTKRAKTRTKAKRKTA |
| 168K | 0.969 | 0.985 | 1668 | KKKKKKKKKKKKKKKKKKKKKKKKKKKKKKKKKKKKKKKKKKKKKKKKKKKKKKKKKKKKKKKKKKK |
| 169L | 0.99 | 0.995 | 2083 | LLLLLLLLLLLLLLLLLLLLLLLLLLLLLLLLLLLLLLLLLLLLLLLLLLLLLLLLLLLLLMLLLLL |
| 170T | 0.971 | 0.985 | 1680 | TTTTTTTTTTTTTTTTTTTTTTTTTTTTTTTTTTTTTTTTTTTTTTTTTTTTTTTTTTTTTTTTTTT |
| 171E | 1 | 1 | 2933 | EEEEEEEEEEEEEEEEEEEEEEEEEEEEEEEEEEEEEEEEEEEEEEEEEEEEEEEEEEEEEEEEEEE |
| 172D | 1 | 1 | 2934 | DDDDDDDDDDDDDDDDDDDDDDDDDDDDDDDDDDDDDDDDDDDDDDDDDDDDDDDDDDDDDDDDDDD |
| 173R | 1 | 1 | 2935 | RRRRRRRRRRRRRRRRRRRRRRRRRRRRRRRRRRRRRRRRRRRRRRRRRRRRRRRRRRRRRRRRRRR |
| 173W | 0.982 | 0.99 | 1911 | WWWWWWWWWWWWWWWWWWWWWWWWWWWWWWWWWWWWWWWWWWWWWWWWWWWWWWWWWWWWWWWWWWW |
| 175N | 1 | 1 | 2936 | NNNNNNNNNNNNNNNNNNNNNNNNNNNNNNNNNNNNNNNNNNNNNNNNNNNNNNNNNNNNNNNNNNN |
| 176K | 0.93 | 0.955 | 1017 | KKKKKKKKKKKKKKKKKKKKKKKKKERKKKKKKKKKKKKEKEKKKKKKKKKKKKKKKKKKKKKKKKK |
| 177P | 0.947 | 0.97 | 1218 | PPPLPPPPPPPPPPPPPPPPPPPPPPPPPPPPPPPPPPPPPPPPPPPPPPPPPPPPPPPPPPPPPPP |
| 178Q | 0.89 | 0.91 | 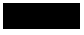 | RQQQQRRQQQQQQQQQQQQQQQQRRQQQRQQQQQRQQQQQQQQQQQQQQQQQQQQQQQQQQQQQQQQ |
| 179K | 0.902 | 0.93 | 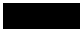 | KKRKKKKKKKKKEKKKKKKKKKKKKKKKKKKKKKKKKKKKKKKKKKKKKKKKKKKKKKKKEKKKKKK |
| 180T | 0.95 | 0.965 | 1239 | TTTTTTTTTTTTTTTTTTTTITTTTTTTTTTTTTTTTTTTTTTTTTTTTTTTTTTTTTTTTITTTTT |
| 181K | 0.947 | 0.97 | 1219 | KKKKKKKKKKKKKKKKKKKKKKKKKKKKKKKKKKKTKKKKKKKKKKKKKKKKKKKKKKKKKKEKKKK |
| 182G | 0.99 | 0.995 | 2084 | GGGGGGGGGGGGGGGGGGGGGGGGGGGGGGGGGGGGGGGGGGGGGGGGGGGGGGGGGGGGAGGGGGG |
| 183H | 0.935 | 0.955 | 1058 | HHHHHHHHHHHHRHHHHHHHHHHHHHHHHHHHHHHHHHHHHRRHHHHHHHHHHHHHHHHHHHRHHHH |
| 184R | 0.894 | 0.91 | 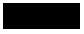 | RRRRRKRRKRKRRKKRRKRKRRRRRKKRRRRRKRKRRRKRRRRRRRRRRKRKRKRRRRRRPRRRRRR |
| 185G | 0.883 | 0.895 | 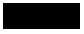 | GGGGGGGGGGEGGGGGGGEGGGGGGGGGGGGGGGGGGGGGGEEGGGGGGGGGGEGGGGGGGGGEGGG |
| 186S | 0.895 | 0.925 | 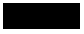 | SSSSSSSSNSSSSSSSSSSSSSSSSSSSSSSSSNSSSSSNSSSSSSSNSSSSSSSSSSSSSSSSNSN |
| 187H | 0.964 | 0.98 | 1559 | HHHHHHHHHHHHHHHHHHHHHHHHHHHHHHHHHHHHHRHHHHHHHHHHHHHHHHHHHHHHHHHHHHH |
| tc,1 Table 3D-2 | | | | |
| 140N | 0.872 | 0.91 | 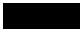 | NSNNNNTNNNNNNNNNNNNNNNSNNNNNNNNNNNNNNNNNNNNNNNNNNNSNNNNNSSNNNNNNNNN |
| 141K | 0.964 | 0.98 | 1558 | KKKKKKKKKKKKKKKKKKKKKKKKKKKKKKKKKKKKKKKKQKKKKKKKKKKKKKKKKKKKKKKKKKK |
| 142V | 0.974 | 0.985 | 1776 | VVVVVVVVVVVVVVVVVVVVVVVVVVVVVVVVVVVVVVIVVVVVVVVVVVVVVVVVVVVVVVVVVVV |
| 143G | 0.982 | 0.99 | 1910 | GGGGGGGGGGGGGGGGGGGGGGGGGGGGGGGGGGGGGGGGGGGGGGGGGGGGGGGGGGGGGGGGGGG |
| 144S | 0.933 | 0.96 | 1035 | FSFSSSSFSSFSSSSSSSSSSSSSSSSSSSSSSSSSSSSSSSSSSSSSSSSSSSPSSSSSSSSSSSS |
| 145L | 0.99 | 0.995 | 2081 | LLLLLLLLLLLLLLLLLLLLLLLLLLLLLLLLLLLLLLLLLLLLLLLLLLLLLLLLLLLLLLLLLLL |
| 146Q | 0.979 | 0.99 | 1900 | QQQQQQQQQQQQQQQQQQQQQQQQQQQQQQQQQQQQQQQQQQQQQQQQQQQQQQQQQQQQQQQQQQQ |
| 147Y | 1 | 1 | 2926 | YYYYYYYYYYYYYYYYYYYYYYYYYYYYYYYYYYYYYYYYYYYYYYYYYYYYYYYYYYYYYYYYYYY |
| 148L | 1 | 1 | 2927 | LLLLLLLLLLLLLLLLLLLLLLLLLLLLLLLLLLLLLLLLLLLLLLLLLLLLLLLLLLLLLLLLLLL |
| 149A | 1 | 1 | 2928 | AAAAAAAAAAAAAAAAAAAAAAAAAAAAAAAAAAAAAAAAAAAAAAAAAAAAAAAAAAAAAAAAAAA |
| 150L | 1 | 1 | 2929 | LLLLLLLLLLLLLLLLLLLLLLLLLLLLLLLLLLLLLLLLLLLLLLLLLLLLLLLLLLLLLLLLLLL |
| 151A | 0.712 | 0.665 | 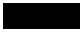 | TTTAAAAAAAATATTATTAAAAAAAAAAAAAAAARTAAATAATAAAAAAASVAATATTTTATATAAT |
| 152A | 0.937 | 0.96 | 1124 | AAAAAVAAVAAAAAAAAAAAAAAAAAAAAVAAAAAAAAAAAAAAAAAAATAAAAAAAAAAAAAAAAA |
| 153L | 0.99 | 0.995 | 2082 | LLLLLLLLLLLLLLLLLLVLLLLLLLLLLLLLLLLLLLLLLLLLLLLLLLLLLLLLLLLLLLLLLLL |
| 154I | 0.677 | 0.675 | 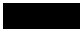 | IILLIILVILIIIVIIVVVIVTTVIIIIVIIIVIILVIIITVITVTIVIIVTIIIIIIIIIITIVII |
| 155T | 0.742 | 0.775 | 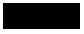 | PTTKKTTTTTTTKQTTTTTTATTTTTTTTTKTTTKKTTKKKKTTTTTTTTTATTTKTKKTTKTTTTT |
| 156P | 0.939 | 0.965 | 1134 | PPPPPPSPPPPPPPPPPPPPPPPPPPPPPPPPPPPPPPPPPSPPPPPPPPPPPPPPPPSPPPPPPPP |
| 157K | 0.826 | 0.865 | 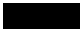 | KKKKKKKKKKKKKKKKKKKKKTKKKKKKKKKTKKKKRKKKTKKKKKKKKKKKKKKKRKKKKKRKRKK |
| 158K | 0.8 | 0.82 | 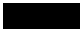 | RRRRKKRKKKKKKKRKKKKKKKKKRKKKKRKQQKKRKKKRKKKRRKKKKKKKKKKQKKKKRKRKKKR |
| 159I | 0.69 | 0.675 | 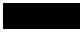 | ITIIIIRIIIIIIIIIIIIRIIIIVIIIIRIRTTIRTITTRIRRIITRIIIIITIIIIITIIIIIII |
| 160K | 0.904 | 0.935 | 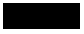 | KKKKKRKKKKRKKKKKKKKKKKKKKKKKKKKKKKKKKKKKKKKKKKKKKKKKKKKKKKKKKKKKKKK |
| 161P | 1 | 1 | 2930 | PPPPPPPPPPPPPPPPPPPPPPPPPPPPPPPPPPPPPPPPPPPPPPPPPPPPPPPPPPPPPPPPPPP |
| 162P | 1 | 1 | 2931 | PPPPPPPPPPPPPPPPPPPPPPPPPPPPPPPPPPPPPPPPPPPPPPPPPPPPPPPPPPPPPPPPPPP |
| 163L | 0.941 | 0.965 | 1144 | LLLLLLLLLLLLLLLLLLLLLLLLMLLLLLLLLLLLLLLLLLLLLLLLLLLLLLLLLLLLLLFLLLL |
| 164P | 1 | 1 | 2932 | PPPPPPPPPPPPPPPPPPPPPPPPPPPPPPPPPPPPPPPPPPPPPPPPPPPPPPPPPPPPPPPPPPP |
| 165S | 0.979 | 0.99 | 1901 | SSSSSSSSSGSSSSSSSSSSSSSSSSSSSSSSSSSSSSSSSSSSSSSSSSSSSSSSSSSSSSSSSSS |
| 166V | 0.945 | 0.965 | 1185 | VVVVVVVVVVVVVVVVVVVVVVVVVVVVVVVVVVVVVVVVVVVVVVVVVVVVVVVVVVVVVVVVVVV |
| 167T | 0.524 | 0.4 | 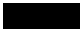 | TTKTAATKATTRTVRAAARTAKKKKAAAAKTTRKAKTVRQATAAATTAKATTKKKRTKKKTKTKAKT |
| 168K | 0.969 | 0.985 | 1668 | KKKKKKKKKKKKKKKKKKKKKKKKKKKKKKKKKKKKKKKKKKKKKKKKKKKKKKKKKKKKKKKKKKK |
| 169L | 0.99 | 0.995 | 2083 | LLLLLLLLLLLLLLLLLLLLLLLLLLLLLLLLLLLLLLLLLLLLLLLLLLLLLLLLLLLLLLLLLLL |
| 170T | 0.971 | 0.985 | 1680 | TTTTTTTTTTTTTTTTAATTTTTTTTTTTTTTTTTTTTTTTTTTTTTTTTTTTTTTTTTTTTTTTIT |
| 171E | 1 | 1 | 2933 | EEEEEEEEEEEEEEEEEEEEEEEEEEEEEEEEEEEEEEEEEEEEEEEEEEEEEEEEEEEEEEEEEEE |
| 172D | 1 | 1 | 2934 | DDDDDDDDDDDDDDDDDDDDDDDDDDDDDDDDDDDDDDDDDDDDDDDDDDDDDDDDDDDDDDDDDDD |
| 173R | 1 | 1 | 2935 | RRRRRRRRRRRRRRRRRRRRRRRRRRRRRRRRRRRRRRRRRRRRRRRRRRRRRRRRRRRRRRRRRRR |
| 173W | 0.982 | 0.99 | 1911 | WWWWWWWWWWWWWWWWWWWWWWWWWWWWWWWWWWWWWWWWWWWWWWWWWWWWWWWWWWWWWWWWWWW |
| 175N | 1 | 1 | 2936 | NNNNNNNNNNNNNNNNNNNNNNNNNNNNNNNNNNNNNNNNNNNNNNNNNNNNNNNNNNNNNNNNNNN |
| 176K | 0.93 | 0.955 | 1017 | KKKKKKKKKKKKKKKKKKKKKKKKKKKKKKKKKEKKKKKKKKKKKKKKKKKKKKKKKKKKKKKKKKK |
| 177P | 0.947 | 0.97 | 1218 | PPHPSPPPPPPPPPPPPPPPPPPSPPPPPPPPPPPPPPPPPPPPPPPPPPPPPPPPPPPPPPPPSPP |
| 178Q | 0.89 | 0.91 | 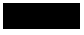 | QQQQQQQQQQQQQQQQQQQQRQQQQQQQHQQQQQQQQRQQQQQQQQQQQQQQQQQQQQQQQQQQHRQ |
| 179K | 0.902 | 0.93 | 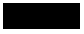 | KKKKKKKKKKKKKKKKKKRKKKKKKKKKNKKKKKKKKKKRKKKKKKKKKKKKKKKKKKKKKKKKKKK |
| 180T | 0.95 | 0.965 | 1239 | TTTTTTTTTTTTTTTTTTTTTTTTTTTTTTTTTTTTTTTTITTITTTTTTTTTTTTITTTTTTTTTT |
| 181K | 0.947 | 0.97 | 1219 | KKKKKKKKKKKKKKKKKKKKKKKKKKKKKKKKKKKKKKKKKKRKKKKRKKKKKKKKKKKKKKKKKKK |
| 182G | 0.99 | 0.995 | 2084 | GGGGGGGGGGGGGGGGGGGGGGGGGGGGGGGGGGGGGGGGGGGGGGGGGGGGGGGGGGGGGGGGGGG |
| 183H | 0.935 | 0.955 | 1058 | HHHHHHHHHHHHHHHHHHHHHHHHHHSHHHHHHHHHHHHHHHHHHHHHHHHHHHHHHHHHHHHHHHR |
| 184R | 0.894 | 0.91 | 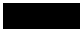 | RRRRRRRRRRRRRRRRRRRRRRRRRRRRRRRRRRRRRRRRRRRRRRRRRRRRRRRRRRRRRRRRRRR |
| 185G | 0.883 | 0.895 | 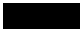 | GGGGGGGGEGEGGGGGGGEGGGGGGGGGGGGEGGGGGGGGGGGGGGGGGGGGGGGGGGGGGGGGGGG |
| 186S | 0.895 | 0.925 | 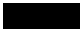 | SNSSSSSSCSSSSSSSSSSSSSSSSSSSSSNSSSSNSSSSSSSSSSSSSSSSSSSSSSSSSSSSSSS |
| 187H | 0.964 | 0.98 | 1559 | HHHHHHHHHHHHHHHHHHHHHHHHHHHHHHHHHHHHHHHHHHHHHHHHHQHHHHHHHHHRHHHHHHR |
| tc,1 Table 3E-1 | | | | |
| 188T | 0.946 | 0.97 | 1190 | #TTTTTTTTTTITTTTTTSTTTTTTTTTTTTTTTTTTTTTTTTTTTTTTTTTTTTTTTTTSTTTTTT |
| 189M | 0.895 | 0.935 | 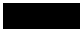 | #MMMMMMMMMMMMMRMMMMMMMMMMMMMMITMMMMMTMMLMMMMMMMMMMMMMMRMMMMMMMMMMMM |
| 190N | 0.832 | 0.88 | 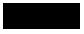 | #NNNNNNNNSNNNNNNNSNNNNNSNNNNNNNNNNNNNHNNNNNNNNNNNNNTNNNDNNHHNNHNNHN |
| 191G | 0.982 | 0.99 | 1912 | #GGGGGGGRGGGGGGGGGGGGGGGGGGGGGGGGGGGGGGGGGGGGGGGGGGGGGGGGGGGGGGGGGG |
| 192H | 0.979 | 0.99 | 1902 | #HHHHHHHHHHHHHHHHHHHHHHHHHHHHHHHHHHHHHHHHHHHHHHHHHQHHHHHHHHHHHHHHHH |
| | | | | #$$$$$$$$$$$$$$$$$$$$$$$$$$$$$$$$$$$$$$$$$$$$$$$$$$$$$$$$$$$$$$$$$$ |
| tc,1 Table 3E-2 | | | | |
| 188T | 0.946 | 0.97 | 1190 | TTTTTTTTTTTTTTTTTTTTTTTTTTTTTTTTTTTTTTTTTTTTTTTTTTTTTTTTTTTTTTTTTTT |
| 189M | 0.895 | 0.935 | 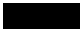 | MMMMMMMMMMMMMMMMMMIMMMMMMMMMMMMMMMMMMLMMMMMMMMMMMMMMMMMMMMMMLLMMMMM |
| 190N | 0.832 | 0.88 | 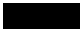 | NNNNNNNHNNNNNNNNNSNNNNNNNNNNNNNNNNNNSNNSNNNNNNNNNNNNNNNNNNNNNNNNNNN |
| 191G | 0.982 | 0.99 | 1912 | GGGGGGGGGGGGGGGGGGGGGGGGGGGGGGGGGGGGGGGGGGGGGGGGGGGGGGGGGGGGRGGGGGG |
| 192H | 0.979 | 0.99 | 1902 | HHHHHHHHHHHHHHHHHHHHHRHHHHHHHHHHHHHHHHHHHHHHHHHHHHHHHHHHHHHHHHHHHHH |
| | | | | $$$$$$$$$$$$$$$$$$$$$$$$$$$$$$$$$$$$$$$$$$$$$$$$$$$$$$$$$$$$$$$$$$$ |
| tc,1 Table 3E-2 | | | | |
| 188T | 0.946 | 0.97 | 1190 | TSTTTTTTTTTTTTTTTTTTTTTTTTRTTTTTTTTTTITTTTTTTTTTTTTTTTTTTTTTTTTTTTT |
| 189M | 0.895 | 0.935 | 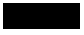 | MMMMLMMMIMMMMMMMMMMMMMMMMMMMMMMMMMMMMMMMMMMMMMMMMMMMMMIMMMMMMMMMMMM |
| 190N | 0.832 | 0.88 | 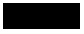 | NDNNNNNNNNNNNHHNNNNNSNHHNNNNNNDNNNNNNNNNNNNNNNNNNSNNNNNNSNNNNNTNNNN |
| 191G | 0.982 | 0.99 | 1912 | GGGGGGGGGGGGGGGGGGGGGGGGGGGGGGGGGGGGGGGGGGGGGGGGGGGGGGGGGGGGGGGGGGG |
| 192H | 0.979 | 0.99 | 1902 | HHHHHHHHHHHHHHHHHHHHHHHHHHHHHHHHHHHHHHHHHHHHHHHHHHHHHHHHHHHHHHHHHHH |
| | | | | $$$$$$$$$$$$$$$$$$$$$$$$$$$$$$$$$$$$$$$$$$$$$$$---$$$$$$$$$$$$$$$$$ |

| | | | |
|---|---|---|---|
| SAMPLE #: 46+HZ,1/42 | | | |
| | Tat | | |
| AA# | Cons. | entropy | Raw data |

TABLE 4A+TL,42

| | | | |
|---|---|---|---|
| 1 | M | 1 | # MMMMMMMMMMMMMMMMMMMMMMMMMMMMMMMMMMMMMMMMMMMMMM |
| 2 | E | 0.903 | # EEEEDEEEEEEEEEEEEEEEEEEEDEEEEEEEEEEDEEEEEDEEEE |
| 3 | P | 0.966 | # PPPPPPPSPPPPPPPPPPPPPPPPPPPPPPPPPPPPPPPPPPPPPP |
| 4 | V | 1 | # VVVVVVVVVVVVVVVVVVVVVVVVVVVVVVVVVVVVVVVVVVVVVV |
| 5 | D | 0.966 | # DDDDDDDDDDDDDDDDDDDDDDDDDDDDDDDDDDDDDDDDDDNDDD |
| 6 | P | 0.966 | # PPPPPPPPPPPPPPPPPPPPPPPPPPPPPPPPPPPPPPPPPPPHPP |
| 7 | R | 0.769 | # RRRRRRRRRRKRRRRRRRRRRRRKRNSRRNRNRRRRRRSNRRSRRR |
| 8 | L | 0.966 | # LLLLLLLLLLLLLLLLLLLLLILLLLLLLLLLLLLLLLLLLLLLLI |
| 9 | E | 1 | # EEEEEEEEEEEEEEEEEEEEEEEEEEEEEEEEEEEEEEEEEEEEEE |
| 10 | P | 1 | # PPPPPPPPPPPPPPPPPPPPPPPPPPPPPPPPPPPPPPPPPPPPPP |
| 11 | W | 1 | # WWWWWWWWWWWWWWWWWWWWWWWWWWWWWWWWWWWWWWWWWWWWWW |
| 12 | K | 0.897 | # KKKKKKKKKKKKKKEKKKKNKKKKKKKRKKKKKKKKKKKKKKKKKK |
| 13 | H | 1 | # HHHHHHHHHHHHHHHHHHHHHHHHHHHHHHHHHHHHHHHHHHHHHH |
| 14 | P | 1 | # PPPPPPPPPPPPPPPPPPPPPPPPPPPPPPPPPPPPPPPPPPPPPP |
| 15 | G | 1 | # GGGGGGGGGGGGGGGGGGGGGGGGGGGGGGGGGGGGGGGGGGGGGG |
| 16 | S | 1 | # SSSSSSSSSSSSSSSSSSSSSSSSSSSSSSSSSSSSSSSSSSSSSS |
| 17 | Q | 1 | # QQQQQQQQQQQQQQQQQQQQQQQQQQQQQQQQQQQQQQQQQQQQQQ |
| 18 | P | 1 | # PPPPPPPPPPPPPPPPPPPPPPPPPPPPPPPPPPPPPPPPPPPPPP |
| 19 | K | 0.819 | # KKRKKKRKRKRKKKKRRKKKRRKKKKKKKKKRKKKKKRKRKKKKKK |
| 20 | T | 0.966 | # TTTTTTTTTTTTTTTTTTTTTTTTTTTTTTTTTTTATTTTTTTTTT |
| 21 | A | 0.887 | # AAPAAAAAAAAAAAAPAAAAAAPAAAAAAAAAAAAADAAAAAAAAA |
| 22 | C | 0.966 | # CCCCCCCCCCCCCCCCCCSCCCCCCCCCCCCCCCCCCCCCCCCCCC |
| 23 | T | 0.848 | # TTTNTTTTTTTTTNTTTTNTTTTNTTTTTTNNTTTTTNTNTTTTTT |
| 24 | N | 0.644 | # KNTNNTPKTTSNNNNNNNNKNNKNNKNNNTNNNTNSNNTNKNNNNK |
| 25 | C | 1 | # CCCCCCCCCCCCCCCCCCCCCCCCCCCCCCCCCCCCCCCCCCCCCC |
| 26 | Y | 0.941 | # FYYYYYYYYYYYYYYYYYYYYYYYYYYYYYYYYYYYYYYYYYYYYF |
| 27 | C | 1 | # CCCCCCCCCCCCCCCCCCCCCCCCCCCCCCCCCCCCCCCCCCCCCC |
| 28 | K | 1 | # KKKKKKKKKKKKKKKKKKKKKKKKKKKKKKKKKKKKKKKKKKKKKK |
| 29 | K | 0.812 | # KKKRQKKKKKKKKKKKKKRKQKKKKKYKKKKKKKRKKKKKKKKRKK |
| 30 | C | 1 | # CCCCCCCCCCCCCCCCCCCCCCCCCCCCCCCCCCCCCCCCCCCCCC |
| 31 | C | 1 | # CCCCCCCCCCCCCCCCCCCCCCCCCCCCCCCCCCCCCCCCCCCCCC |
| 32 | F | 0.701 | # YWLLFFFFYFFFFLFLFFLLFFLFLFLLFFYFFFFFFFLFLFFFLF |
| 33 | H | 1 | # HHHHHHHHHHHHHHHHHHHHHHMHHHHHHHHHHHHHHHHHHHHHHH |
| 34 | C | 1 | # CCCCCCCCCCCCCCCCCCCCCCCCCCCCCCCCCCCCCCCCCCCCCC |

TABLE 4B+TL,42

| | | | |
|---|---|---|---|
| 35 | Q | 0.966 | # QQQQQQQQQQQQQQQQQQQQQQQQQQQQQQQYQQQQQQQQQQQQQQ |
| 36 | V | 0.941 | # VVVVVVVVVVVVVVVVVVVVVVVVVVVVVVVAVVVVVVVVVVAVVV |
| 37 | C | 1 | # CCCCCCCCCCCCCCCCCCCCCCCCCCCCCCCCCCCCCCCCCCCCCC |
| 38 | F | 1 | # FFFFFFFFFFFFFFFFFFFFFFFFFFFFFFFFFFFFFFFFFFFFFF |
| 39 | T | 0.591 | # MLMTIIIMIIIITMTTIITIILMTMTTTMILTTTTTTTITMIIMIM |
| 40 | T | 0.701 | # TKTKRTTTTTTKTKKRTTKTTRTKTTRRTTTRKKTTTRTKTTTTTT |
| 41 | K | 0.966 | # KKKKKKKKKQKKKKKKKKKKKKKKKKKKKKKKKKKKKKKKKKKKKK |
| 42 | G | 0.838 | # AGGGGGAGGGGGGGGGGAGGGGGGAAGGAGGGAAAGGGGGGGGGGG |
| 43 | L | 1 | # LLLLLLLLLLLLLLLLLLLLLLLLLLLLLLLLLLLLLLLLLLLLLL |
| 44 | G | 0.966 | # GGGGGGGGGGGGGGGSGGGGGGGGGGGGGGGGGGGGGGGGGGGGGG |
| 45 | I | 1 | # IIIIIIIIIIIIIIIIIIIIIIIIIIIIIIIIIIIIIIIIIIIIII |
| 46 | S | 0.966 | # SSSSSSSSSSSSSFSSSSSSSSSSSSSSSSSSSSSSSSSSSSSSSS |
| 47 | Y | 0.907 | # NYYYYYYYYYYYYYYYYYYYYYYYNVYYYHYYYYYYYYYYYYYYYY |
| 48 | G | 1 | # GGGGGGGGGGGGGGGGGGGGGGGGGGGGGGGGGGGGGGGGGGGGGG |
| 49 | R | 0.966 | # RRRRRRRRRRSRRRRRRRRRRRRRRRRRRRRRRRRRRRRRRRRRRR |
| 50 | K | 1 | # KKKKKKKKKKKKKKKKKKKKKKKKKKKKKKKKKKKKKKKKKKKKKK |
| 51 | K | 1 | # KKKKKKKKKKKKKKKKKKKKKKKKKKKKKKKKKKKKKKKKKKKKKK |
| 52 | R | 1 | # RRRRRRRRRRRRRRRRRRRRRRRRRRRRRRRRRRRRRRRRRRRRRR |
| 53 | R | 0.941 | # RRRKRRRRKRRRRRRRRRRRRRRRRRRRRRRRRRRRRRRRRRRRRR |
| 54 | Q | 0.966 | # QQQQQQQQQQQQQQQQQQQQQQQQQQQQQQQQQQQQQQQQQQQQKQ |
| 55 | R | 1 | # RRRRRRRRRRRRRRRRRRRRRRRRRRRRRRRRRRRRRRRRRRRRRR |
| 56 | R | 1 | # RRRRRRRRRRRRRRRRRRRRRRRRRRRRRRRRRRRRRRRRRRRRRR |
| 57 | R | 0.966 | # RRRRRRRRRRRRRRRRRRRRRRRRRRRRRRGRRRRRRRRRRRRRRR |
| 58 | A | 0.579 | # APPSAAAAPAPSTAAAAAAATLASTAATADPAAAAAATAPAAPSST |
| 59 | P | 0.869 | # PPPPPPPPPPPPPPPPPHPPPPPPPPPPHPPPHPPPHPPPPPPPPD |
| 60 | Q | 0.809 | # QQQPEQQQPQQPQQEQQQQPQEQQQQQQQQQQQEQQQQQQQQQQQQ |
| 61 | D | 0.718 | # DDDDDDNDDDDDDDDDDNDDSDDDNGDDNDGDDDDDNDDDDEDNHD |
| 62 | S | 0.883 | # SGSSSSSSSSNSSSSSSSSSSSNSSSSSSSSSSSSSSSSSSGSSSS |
| 63 | Q | 0.78 | # EQKQQEQQQKQQKQQQQQKQKQKEQQQQQQQQQQEQQQQQQQQQQQ |
| 64 | T | 0.771 | # NANNTTTTTTTTTTTTTTTTTINTTATNTTTTNTTTNTTTTTTTTT |
| 65 | H | 0.897 | # HHHHHHHHHHHHHHHHHHHRHHHHHHHHHNHHHHHHHHHHHHHHDH |
| 66 | Q | 1 | # QQQQQQQQQQQQQQQQQQQQQQQQQQQQQQQQQQQQQQQQQQQQQQ |
| 67 | V | 0.684 | # VVVVVVVIAVVVVAVVVAVVAVVVAAVVAVVAAVVVVLASAVVDAV |
| 68 | S | 0.853 | # SSSSSSSYSSSPSSSSSSSSSSSSDSSSSSSSSSSSPSSSSPSSSS |
| 69 | L | 1 | # LLLLLLLLLLLLLLLLLLLLLLLLLLLLLLLLLLLLLLLLLLLLLL |
| 70 | S | 0.828 | # SSSPSSSPSPSPSSSPSSSSSPSSSSSSSPSSSPSPPSSSSSSSSS |
| 71 | K | 0.931 | # KKKKKKKKKQKKKKKKKKKKKKKKKEKKKKKKKKKKKKKKKKKKKK |
| 72 | Q | 1 | # QQQQQQQQQQQQQQQQQQQQQQQQQQQQQQQQQQQQQQQQQQQQQQ |

TABLE 4C+TL,42

| | | | |
|---|---|---|---|
| 73 | P | 0.931 | # PPPPSPPPPPPPPPPPRPPPPPPPPPPPPPPPPPPPPPPPPPPPPP |
| 74 | A | 0.657 | # ASAAATATTSSAAAASATATATTATTATTATASAAAATSTTSSTAS |
| 75 | S | 0.941 | # SSSSPSSSSSSSSSSSSSSSSSSSSSSSSSSSSPSSSSSSSSSSSS |
| 76 | Q | 0.907 | # QQQKQQQQQQQQQQQQQQQQQQRQQQQQQQQQQQQQQQQQQQQQQR |
| 77 | P | 0.633 | # PPLPPHPPPPPPPPPQPPPPFPAPPLPPSPPSTFPAPPPLPFPPPP |
| 78 | R | 0.883 | # RRRRRRRRRRRRGRRRGRRRQQRRRRRRRRRRRRRRRRRRRRRRRR |
| 79 | G | 0.966 | # GGGEGGGGGGGGGGGGGGGGGGGGGGGGGGGGGGGGGGGGGGGGGG |
| 80 | D | 1 | # DDDDDDDDDDDDDDDDDDDDDDDDDDDDDDDDDDDDDDDDDDDDDD |
| 81 | P | 0.966 | # PPPPPPPPPPPPPPPPPPPPPPPPQPPPPPPPPPPPPPPPPPPPPP |
| 82 | T | 0.907 | # TTTTTTATTTTTTATDTTTTTTTTTTTTTTTTTTTTTTTTTTTTTT |
| 83 | G | 0.966 | # GGGGGGGGGGGGGGGSGGGGGGGGGGGGGGGGGGGGGGGGGGGGGG |
| 84 | P | 0.873 | # PPPPPPPQPPPQQPPPPPPPPPQPQQPPPPPPPPPPPPPPPPPPPP |
| 85 | K | 0.826 | # KEKKKKKKKKKKKKKKKKKEKKEKKTKEKKKTKKKKKKKTKKKKKK |
| 86 | E | 0.966 | # EEEEEEEEEEEEEEEKEEEEEEEEEEEEEEEEEEEEEEEEEEEEEE |
| 87 | S | 0.826 | # SSSSSPSSSSSSSSSSS$SSSSSSSSSS$SSSPSSSSSSSSSQSSQ |
| 88 | K | 0.873 | # KKKKKKKKKKKKKKKKK-KKKKKKTKQK-KKKKKKKKKKKKKKKKK |
| 89 | K | 0.829 | # KKKKKKKKKKKKKKKKK-KKKKEKKEKT-KEKKKKKKKKKKKKKKK |
| 90 | K | 0.907 | # KKKKKKKKKKKKKKKKK-KKKKKKKKKK-KKKEKKKKKKKKKKKKK |
| 91 | V | 0.941 | # VVVVVVVVVVVVVVVVV-VVVVVVVVVV-VVVVVVVVVVVVVVVVV |
| 92 | E | 0.941 | # EEEEEEEEEEEEEEEEE-EEEEEEEEEE-EEEEEEEEEEEEEEEEE |
| 93 | R | 0.689 | # SRRRSRRRRRRRRSTRR-RRGSKRRRSR-RRRRRRRRTRRRRRRTR |
| 94 | E | 0.849 | # EAEEKEEEEEEEEEEEE-EEEKEEEEEE-EEEEEEEEEEEEEEEEE |
| 95 | T | 0.863 | # TATTTTTATTTTTTTTT-TTTTTTTTTT-TTTATTTTTTTTTTTTT |
| 96 | E | 0.883 | # EEEEEEEEEEEEEEEEE-EEEEVEEEEE-EEEEEEEEEEEEVEEEE |
| 97 | T | 0.797 | # TATTTTTAAATTTTTAT-TTTTVTTTTT-TTTTTTTTTTTTTTTTT |
| 98 | D | 0.73 | # DDYDNDDADDDHDDDDD-DDHDDDDDDD-DDDDHDDDDDDDWDDDD |
| 99 | P | 0.883 | # PQPPPPPPPPPPPPPPP-PPPPPPPPPP-PPPPPPPPPPPPPPPPQ |
| 100 | V | 0.276 | # VKDGSVRLLVLGVRIFV-EVGSVVNCSV-VAFLVDVVCDVVEVEVG |
| 101 | D | 0.514 | # DNDTDDDDDDDRHDDDN-DADDTDDDAN-HVDDDHDHDDHDSHDHN |
| 102 | Q | 0.873 | # $$$$$$$$$$$$$$$$$-$$$W$$Q$$$-$Q$$$$$$$$$$C$$$$ |
| 103 | E | | |

SAMPLE #: 47

| | | | |
|---|---|---|---|
| | Rev | | |
| AA# | Cons. | entropy | Raw Data |

| | | | |
|---|---|---|---|
| 1 | M | 1 | # MMMMMMMMMMMMMMMMMMMMMMMMMMMMMMMMMMMMMMMMMMMMMMM |
| 2 | A | 1 | # AAAAAAAAAAAAAAAAAAAAAAAAAAAAAAAAAAAAAAAAAAAAAAA |
| 3 | G | 0.966 | # GGGGGGGGGGGVGGGGGGGGGGGGGGGGGGGGGGGGGGGGGGGGGGG |
| 4 | R | 1 | # RRRRRRRRRRRRRRRRRRRRRRRRRRRRRRRRRRRRRRRRRRRRRRR |
| 5 | S | 0.966 | # SSSSSSSSSSSSSSSSSSSSSSSSSSSSSSSRSSSSSSSSSSSSSSS |
| 6 | G | 0.966 | # GGGGGGGGGGGGGEGGGGGGGGGGGGGGGGGGGGGGGGGGGGGGGGG |
| 7 | D | 0.909 | # DDDDNDDDDNDDDDDDDDDDDDDDDDDDDDDDDDDDDDDDDDDDEDD |
| 8 | S | 0.942 | # SNSSSSSSSSSSSSSSSSSSSSSSSSSNSSSSSSSSSSSSSSSSSSS |
| 9 | D | 0.922 | # DEDDDDDDDDDEDDDDDDDDDDDDDDDDDDDDDDDDEDDDDDDDDDD |
| 10 | E | 0.966 | # EEEEEDEEEEEEEEEEEEEEEEEEEEEEEEEEEEEEEEEEEEEEEEE |
| 11 | E | 0.806 | # EEDDDEEEEDEDDEEEEEEEEEDEDEEEEEEDEEEEEEEDEEDDDEE |
| 12 | L | 0.933 | # LLLLLLLLLLLLLLLLLLLLLLFLLLLLLLLLLLLLLLLLLLLLLLI |
| 13 | L | 0.904 | # LLLLLLLLLLLLLLLLLLILLLLLLLLLLILLLILLLLLLLLLLLIL |
| 14 | K | 0.739 | # QKKKQKKKKQKKQKKKKKRKKKKRKRKKRRKKRKKKKKKRKKKKNKK |
| 15 | T | 0.889 | # TTTTTTTTTTTTTTTTTTTTTATTTTTTATTATTTTTTTTTKTTTTT |
| 16 | V | 0.871 | # VVVVVVVVVVVIVVIVVVVVVVVIVVIVAVVVVVVVVVVVVVVVVVV |
| 17 | R | 0.966 | # KRRRRRRRRRRRRRRRRRRRRRRRRRRRRRRRRRRRRRRRRRRRRRR |
| 18 | L | 0.842 | # LILIILLLLLLLLLLLLLLLLLFILLLILLLLLILLLLLLLLLLLLL |
| 19 | I | 0.966 | # VIIIIIIIIIIIIIIIIIIIIIIIIIIIIIIIIIIIIIIIIIIIIII |
| 20 | K | 0.933 | # KKKKKKKKKKKKKKKKKKKKKKKKKKKKKKKKKKKKKNKKKRKKKKK |
| 21 | F | 0.649 | # FFFFFFFFSLSFFLLFFFLFFLFFFLFFLLFSLHFFFFLVLFFILFF |
| 22 | L | 0.942 | # LLLLLLLLILLLLLLLLLLLLLLLLILLLLLLLLLLLLLLLLLLLLL |
| 23 | Y | 1 | # YYYYYYYYYYYYYYYYYYYYYYYYYYYYYYYYYYYYYYYYYYYYYYY |
| 24 | Q | 0.966 | # QQQQQQQQQQHQQQQQQQQQQQQQQQQQQQQQQQQQQQQQQQQQQQQ |
| 25 | S | 1 | # SSSSSSSSSSSSSSSSSSSSSSSSSSSSSSSSSSSSSSSSSSSSSSS |
| 26 | N | 1 | # NNNNNNNNNNNNNNNNNNNNNNNNNNNNNNNNNNNNNNNNNNNNNNN |
| 27 | P | 0.966 | # PPPPPPPPPPPPPPPPPAPPPPPPPPPPPPPPPPPPPPPPPPPPPPP |
| 28 | P | 0.862 | # PLPPPPPLPPPPPLPPPPPPPLPLPPPPLPPPPPPPPPPPPPPPLPP |
| 29 | P | 1 | # PPPPPPPPPPPPPPPPPPPPPPPPPPPPPPPPPPPPPPPPPPPPPPP |
| 30 | S | 0.655 | # SNSSSSSSNSNNNSRSSSNNSSNESNNNSNNSSKSSKSSSNSSSSGS |
| 31 | P | 0.681 | # PLPSPPTPPPPPPPPPNQPPQSPPPPPPSPPPPPSPPPPSPSLPLPP |
| 32 | E | 0.942 | # EEEEEEEEEEEEEEEEEEEEEKKEEEEEEEEEEEEEEEEEEEEEEEE |
| 33 | G | 0.966 | # GGGGKGGGGGGGGGGGGGGGGGGGGGGGGGGGGGGGGGGGGGGGGGG |
| 34 | T | 1 | # TTTTTTTTTTTTTTTTTTTTTTTTTTTTTTTTTTTTTTTTTTTTTTT |
| 35 | R | 0.966 | # RRRRRRRRRRRRRRRRPRRRRRRRRRRRRRRRRRRRRRRRRRRRRRR |
| 36 | Q | 0.966 | # QQQQQQQQQQQQQQQQTQQQQQQQQQQQQQQQQQQQQQQQQQQQQQQ |
| 37 | A | 1 | # AAAAAAAAAAAAAAAAAAAAAAAAAAAAAAAAAAAAAAAAAAAAAAA |
| 38 | R | 1 | # RRRRRRRRRRRRRRRRRRRRRRRRRRRRRRRRRRRRRRRRRRRRRRR |
| 39 | R | 1 | # RRRRRRRRRRRRRRRRRRRRRRRRRRRRRRRRRRRRRRRRRRRRRRR |
| 40 | N | 1 | # NNNNNNNNNNNNNNNNNNNNNNNNNNNNNNNNNNNNNNNNNNNNNNN |
| 41 | R | 1 | # RRRRRRRRRRRRRRRRRRRRRRRRRRRRRRRRRRRRRRRRRRRRRRR |
| 42 | R | 1 | # RRRRRRRRRRRRRRRRRRRRRRRRRRRRRRRRRRRRRRRRRRRRRRR |
| 43 | R | 1 | # RRRRRRRRRRRRRRRRRRRRRRRRRRRRRRRRRRRRRRRRRRRRRRR |
| 44 | R | 1 | # RRRRRRRRRRRRRRRRRRRRRRRRRRRRRRRRRRRRRRRRRRRRRRR |
| 45 | W | 1 | # WWWWWWWWWWWWWWWWWWWWWWWWWWWWWWWWWWWWWWWWWWWWWWW |
| 46 | R | 1 | # RRRRRRRRRRRRRRRRRRRRRRRRRRRRRRRRRRRRRRRRRRRRRRR |
| 47 | E | 0.735 | # GAEEEAEEEEEEEAAQEEEEEEAREEEEEEAEEEEEEQEEEEEEQEE |
| 48 | R | 1 | # RRRRRRRRRRRRRRRRRRRRRRRRRRRRRRRRRRRRRRRRRRRRRRR |
| 49 | Q | 1 | # QQQQQQQQQQQQQQQQQQQQQQQQQQQQQQQQQQQQQQQQQQQQQQQ |
| 50 | R | 0.966 | # RRRRRRRRRRRRRRRRRRRRRRRWRRRRRRRRRRRRRRRRRRRRRRR |
| 51 | Q | 0.823 | # QQQHQQQQQQQQHQQQQQQQQHQWQQHQQQQQQRHQQQQQQQQQQQQ |
| 52 | I | 0.942 | # IIIIIIIILIIIIIIIIIIIIIIIIILIIIIIIIIIIIIIIIIIIII |
| 53 | R | 0.642 | # QRRQQRRRHHQHQRQQRRHRRRHQRRHRRHRRRHRRRRRRRNRRQKR |
| 54 | S | 0.51 | # SSRTVQSESSSSAESSSSSKFAQSSTSSASARSSSTSATSSSSKSAS |
| 55 | I | 0.823 | # LIIILIIIIIIIVIIIIIIIIIILIIIIIILCIIIIIIIIILIIIII |
| 56 | S | 0.966 | # SSSSSSSSSSSSSSSSSSSSSSGSSSSSSSSSSSSSSSSSSSSSSSS |
| 57 | G | 0.532 | # ADGNEGDGAAATADGEEGEGENEGEGEGGEGEGAAEGGEGEAEGGGE |
| 58 | W | 0.754 | # WWWW$WRRRRRRWRWRRWRWWWWWRWRWWRWWWWWWWWRWRWRWWWR |
| 59 | I | 0.942 | # IIIIIIIIIIIIIIIIIIIIIIIIIIIIIIIIILIIIIILIIIIIII |
| 60 | L | 0.942 | # LILLLLLLLLLLILLLLLLLLLLLLLLLLLLLLLLLLLLLLLLLLLL |
| 61 | S | 0.744 | # SRSSDSSSSSSSSVSSSSGSSSSNSSSSSSSDSSSSSSSSSSGNSSD |
| 62 | T | 0.757 | # TTTAATTTTTTTTTTTTNTTNTATTNTNTTNTTTNTNTTNTTTTNNT |
| 63 | Y | 0.572 | # RHFHYYYFFFFFHYSYFYYYHHFHYYYYSYYYYCYHYFYYFHFYHYY |
| 64 | L | 1 | # LLLLLLLLLLLLLLLLLLLLLLLLLLLLLLLLLLLLLLLLLLLLLLL |
| 65 | G | 1 | # GGGGGGGGGGGGGGGGGGGGGGGGGGGGGGGGGGGGGGGGGGGGGGG |
| 66 | R | 0.922 | # RRRRRRRGRRRRRGRRRRRRRRRRRRRRRRRRRRRRRRRRRRRRRRG |
| 67 | P | 0.738 | # SPPPPPPPPPPPSSSPPPSSPPPPSPPPPSPSSSPSIPPPSPFPPPS |
| 68 | A | 0.691 | # TTAAAAATEEEETTAEAAAAAAAAAAEAAAAVAAAAAAATAAETAAT |
| 69 | E | 0.832 | # QEEEEEEEEEEKEEEQEEEEEEEEEEEEQEEDEEEEEEEEEEEEDEE |
| 70 | P | 0.933 | # PPPPPPPPSPPPPPPPPPPPPPPPPPPPPPPPPPPPPPPPPPPPAPP |
| 71 | V | 0.966 | # VVVVVVVVVVVVVVVVVVVVVVVVVVVVVVVVVVVVVVVVVVVVVVG |
| 72 | P | 0.966 | # PPPPPPPPPPPPPPPPPPPPPPPPPPPPPPPQPPPPPPPPPPPPPPP |
| 73 | L | 0.942 | # LLLLLLLLLLLLLLLLLLLLLLLLLLLLLLLLLLLLLLLFLLLLLFL |
| 74 | Q | 0.832 | # QQHQQQQQQQQPQQQPQQQQQQQQQQPQQQQQQQQQQQQQQQPPQHQ |
| 75 | L | 1 | # LLLLLLLLLLLLLLLLLLLLLLLLLLLLLLLLLLLLLLLLLLLLLLL |
| 76 | P | 1 | # PPPPPPPPPPPPPPPPPPPPPPPPPPPPPPPPPPPPPPPPPPPPPPP |
| 77 | P | 1 | # PPPPPPPPPPPPPPPPPPPPPPPPPPPPPPPPPPPPPPPPPPPPPPP |
| 78 | L | 0.889 | # LLLLLLLLLLLLLLLLLLLLLLILLLLLLLILLL-LLLLLLLLLLIL |
| 79 | E | 0.933 | # EEEEEEEEEEEEEEEEEEEEEEEEEEEEEEEEEEQEEEEEEEEDEEE |
| 80 | R | 0.966 | # RRRRRRRRRRRRRRRRRRRRRRRRRRRRRRRRRRRRRRRRRRKRRRR |
| 81 | L | 1 | # LLLLLLLLLLLLLLLLLLLLLLLLLLLLLLLLLLLLLLLLLLLLLLL |
| 82 | T | 0.904 | # TNTTNTTTTTTTTTTTTTTNTTTTTTTTTTTTTTTTTTTTNTTTTTT |
| 83 | L | 1 | # LLLLLLLLLLLLLLLLLLLLLLLLLLLLLLLLLLLLLLLLLLLLLLL |
| 84 | D | 0.909 | # DDDDGDDDDDDDDDDDDDDDDDDDDDDDDDDDDDDDDDDDGDDDDDN |
| 85 | C | 0.942 | # CCCCCCCCCCCCCCCCCCCCCCCCCCCCCCCSCCCCCCCCCCCCCSC |
| 86 | N | 0.682 | # SSNSSSNNNNNNNSNSSNNSNSDNDDSNNNSSSSSSNSNNNSNKNNN |
| 87 | E | 0.904 | # EEEEEKEKEEEEEKEEEEEEEKEEEEEEEEEEEEEEEEEEEEEEEEE |
| 88 | D | 0.966 | # DDDDDDDDDDDDDDDDDDDDDDDEDDDDDDDDDDDDDDDDDDDDDDD |
| 89 | C | 0.966 | # CCCCCCCCCCCCCCLCCCCCCCCCCCCCCCCCCCCCCCCCCCCCCCC |
| 90 | G | 1 | # GGGGGGGGGGGGGGGGGGGGGGGGGGGGGGGGGGGGGGGGGGGGGGG |
| 91 | T | 0.942 | # TTTTTTTTTTTTTTTTNTTTTTTTTTTTTTTTNTTTTTTTTTTTTTT |
| 92 | S | 0.966 | # SSSSSSSSSSSPSSSSSSSSSSSSSSSSSSSSSSSSSSSSSSSSSSS |
| 93 | G | 0.966 | # GGGGRGGGGGGGGGGGGGGGGGGGGGGGGGGGGGGGGGGGGGGGGGG |
| 94 | T | 0.966 | # TTTTTTTTTTTTTTTTTTTTTTTTTTTTTTTTATTTTTTTTTTTTTT |
| 95 | Q | 0.966 | # QQQQQQQQRQQQQQQQQQQQQQQQQQQQQQQQQQQQQQQQQQQQQQQ |
| 96 | G | 1 | # GGGGGGGGGGGGGGGGGGGGGGGGGGGGGGGGGGGGGGGGGGGGGGG |
| 97 | V | 0.799 | # VVVVEVLVMMVMVVAVVVVVVVVVVVVVVVVVVVVVVVVVVVVVVLV |
| 98 | G | 1 | # GGGGGGGGGGGGGGGGGGGGGGGGGGGGGGGGGGGGGGGGGGGGGGG |
| 99 | S | 0.731 | # NNNNSGSSSSSSDSNSSSSSSSSNSSSNSSSSSSSSSSSSSDSSTNS |
| 100 | P | 0.909 | # PPPPPPPPPPAPPPSPPPPPPPPPPPPPPPPPPPPPPPPPPPPSPPP |
| 101 | Q | 0.942 | # QQQQQQQQQQQQQQQQQQQEQQQQQQQQQQQQQQQQQQEQQQQQQQQ |
| 102 | I | 0.789 | # VIVILVIVIIIIIIVIVIIIIIVIIIIIIIIVIIIIIVIIVIIIIVI |
| 103 | L | 0.933 | # LSLLLLLLLLLLLLLLLLLLLLLLLFLLLLLLLLLLLLLLLLLLLLL |
| 104 | V | 0.909 | # VVVVVVVVVVVVVVVVVVVVVVVVVVVVVVVVVVVLVVVVVGVLVVV |
| 105 | E | 0.966 | # EEEEEEEEEEEEEEEEEEEEKEEEEEEEEEEEEEEEEEEEEEEEEEE |
| 106 | S | 0.909 | # SSSSYSSSSSSPSSSSSSSSSSSSSSSSSPSSSSSSSSSSSSSSSSS |
| 107 | P | 1 | # PPPPPPPPPPPPPPPPPPPPPPPPPPPPPPPPPPPPPPPPPPPPPPP |
| 108 | A | 0.698 | # AAAATATTPTTKTATAATTATAAAATTATTAAAATATATPTTAAAAA |
| 109 | V | 0.904 | # VVVVVVVVVVIVVVVVVIVVVVVVVVVVVIVVVVVVVVVVVVIVVVV |
| 110 | L | 1 | # LLLLLLLLLLLLLLLLLLLLLLLLLLLLLLLLLLLLLLLLLLLLLLL |
| 111 | E | 0.735 | # EEEEKEEEEGEEGDEEEDEEEEDEEDDEEEEEDEEGEEEDEDEEEED |
| 112 | S | 0.785 | # SSSSSSSSTSSPSSPSPSSPSSPSSSSSSSSSSSSSSSSSPIPPSSS |
| 113 | G | 0.966 | # GGGGGGGGGGGGGGGGGGGGGGGGGGGGGGGGGGGGGGGGGEGGGGG |
| 114 | T | 0.838 | # STTTTAATTTTTTTTTTTTTTTTTTTTTATTATTTTTTTTTLTTTTT |
| 115 | K | 0.786 | # KKKKKAKKTTQAKKKKKKKKKKKKKKKKKKKKKKKKKKKKKSKKKEK |
| 116 | E | 0.933 | # EEEEEEEEEEEEEEEEEEEEEEEEEEEEEEEEEEEEEEEEETEEEEK |
| 117 | E | 0.871 | # $$$$$$$$EEEE$$$$$$$$$$XX$$X$$$$$$$-$$$$$$R$$$$$ |

SAMPLE #: 101+HL,47

| | | | |
|---|---|---|---|
| | Vpu | | |
| AA# | Cons. | entropy | Raw Data |

TABLE 6A+TL,47

| | | | |
|---|---|---|---|
| 1 | M | 0.909 | # IMMMMMMMMMMMMMMMMMMMMTIMMMMMMMMMIMMMMMMMMMMMMMMMMMMMMMM |
| | | | MMMMMMMMMMMMMMMMMMMMMMMMMMMMMMMMMIMMMLMMMMMMMM |
| 2 | Q | 0.763 | # VNLQQQQQQQQQQQQPQQQLQQYQNQIQQQQL-QQQQQQQQQQQQQQQQXQQQQQ |
| | | | QQQQQQNQQQPQQQQQQQQQQQQQQQQQQQQQQQQQQQQ-LQQQXQ |
| 3 | S | 0.641 | # STSSPAAPPSSPSAPSPSSSSPSPSPPSSSSP-SPPTPSPPSSPPPPSLSSPSPS |
| | | | SSSSPPSASPPPFSPPPSPSLPSSPPPSSSPSPSSPPSPPSSPSSP |
| 4 | L | 0.827 | # LLLLLLLLLLLLLLLLLLLLLILLLLLLLLLL-LILLLSLLSLLLILLLVLLLLL |
| | | | LLLLPLLLLLLLLLLLLLLLIILLVLLLLLLLLLLLLLILLLLLLL |
| 5 | Q | 0.416 | # QTKQIQQQQHQQITHXQQQQQPQVQXQVQQQQQQQQQQEEYEEQEQH$AQNQQVE |
| | | | QQKPLQNKAQQEVQHAQHQEL-QEQVEEQEVQQEQQQQQYQQVQEN |
| 6 | I | 0.917 | # IIIIIIIIIIIIIIIIIIVIIIIVIIIIIIIIIIIIIIIIIIIIIIIIIIIIIVI |
| | | | IIIIIIIIIIVIIIIIIITIIIIIIIIIIIIIIIIIIVIIIIIIII |
| 7 | L | 0.576 | # VALVVLLVVVVVLLVLALAALVLASVAIVVV$LLLLVLLLVLLLLLVALLVVLAL |
| | | | SLVAYSLVLLLLLVLASVSLVVVLLLLLLVALLLLLLLALLLLLFA |
| 8 | A | 0.846 | # AATAGAAAAAAAAAAAAAAAAAALAAAAAAAATAAAAAASTAAAVAAAAAAAAAA |
| | | | AAAAAAGAAAAAAAAAAAAATAAAAAAAAAAASAAAAAAAAAAAAA |
| 9 | I | 0.891 | # IIIIIIILLIIIIIIIIIIIVIIVIIIIIIIIIIIIIIIIIIIIIIIIILIIIII |
| | | | IIIIIIIIIIVIIIIIIIIIIILIIIIIIIIIIIIIIIIVIIIIII |
| 10 | V | 0.92 | # VVVVIVVVVVVVVVVVVVVVVVVAVVVVVVVVVVVVVVVVVVVVVVVVVVVVVVV |
| | | | VVVVVVXAVVVVVAVVVVVVVVVVVVVVVVVVVVVVVVVVVVVVVV |
| 11 | A | 0.932 | # AAAAAAAAAAAAAAAAAAAAAAAAAAAAAAAAAAAAAAAAAAAAAAAAGAAAAAA |
| | | | AAAAAAAAAAAAASAAAAAAAAAAAAAASEAAAAAAAAAAAAAAAA |
| 12 | L | 0.964 | # LLLLLLLLLLLLLLLLLLLLLLLLLXLLLLLLLLLLLLLLLLLLLLLLLLLLLLL |
| | | | LLLLLLLLLLLLLLLLLLLLLLLLLLLLLLLLLLLLLLLLLLLFLL |
| 13 | V | 0.956 | # VVVVVVVVVVVVVVVVVVVVVVVIVVVVVVVVVVVVVVVVVVVVVVVVVVVVVVV |
| | | | VVVVVVVVVVVVVVVVVVIIVVVVVVVVVVVVVVVVVVVVVVVVVV |
| 14 | V | 0.95 | # VVVVVVVVVVVVVVVVVVVVVVVVVVVVVVVVVVVVVVVVVVVVVVVVVVVVVVV |
| | | | VVVVVVVVIVVVVVVVVVVVVVVVIVVVVVVVVVVVVVVLVVVVVV |
| 15 | A | 0.762 | # VAAAAAAAAAVAAVAAVAVAAAVVAVVAAAAVAAAVVVAAAAAAVAAVAAAAGVA |
| | | | VVAAAAAVAVAAAVAVAAAATAATVVAAVAAAAAAAAAVAAAAAVA |
| 16 | A | 0.482 | # TAGAAIIIIITAAAIVAAAAAITTATAXTTTAGIATATIFIIIAAATAATTAAAI |
| | | | ATVAAAAGGGAIAGAGAILALIGIGSAAAAGAAIAAGTGAAGLLTT |
| 17 | I | 0.968 | # IIIIIIIVVIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIII |
| 18 | I | 0.856 | # IIIIIIIIIIIILIILIIIIIIIIVIIIIIIIVIIIIIIILIIIIIIILIIIIIL |
| | | | IILIIILIIIILLIIIILIIIIIIIIILIIIIIIIIIIIIIIIIII |
| 19 | A | 0.945 | # AVAAAAAAAAAAAAAAAAAVAAAAAAAAAAAAAAAAAAAAAVAAAAAAAAAAAAA |
| | | | AAAAAAAAAAAAAAAAAAAAAAAAAAAAAAAAAAAAAAAAAAVAAA |
| 20 | I | 1 | # IIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIII |
| 21 | V | 0.899 | # VVIVVVVIIVVVVVVVVVVVVVVVVVVVVVVVVVVVVVVVIVVVVVVVVVVVIVV |
| | | | VVVVVVVVIVVVVVVVVVVVVAVVVVVVVVVVVVVVIVVVVVVVVV |
| 22 | V | 1 | # VVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVV |
| 23 | W | 0.968 | # WWWWWWWWWWWWWWWWW-WWWWWWWWWWWWWWWWWWWWWWWWWWWWWWWWWWWWW |
| | | | WWWWWWWWWWWWWWWWWWWWWWWWWWWWWW$WWWWWWWWWWWWWWW |
| 24 | S | 0.682 | # TTSSTTTSSSTTTTSTS-TTSSTSTSTSTTTTISSSSSSTSSTSTSSSFSSSTSX |
| | | | SSSTSTSSSTTSTSTSSSTSSSSSATTTTTSSSSTTST-TSSSTXT |
| 25 | I | 0.968 | # IIIIIIIIIIIIIIIIIXIIIIIIIIIIIIIIIIIIIIIIIIIIIIIIIIIIIII |
| | | | IIIIIIIIIIIIIIIIIIIIIIIIIIIIIIIIIIIIII-IIIIIII |
| 26 | V | 0.936 | # VVVVVVVVVVVVVVVVVVVVVVVVVVVVVVVVVVVVVVVVVVVVVVVVVVVVVVV |
| | | | VVVVVVVVVVVVVVVVAVVVVVVAVVVVVVVVVVVVVV-VVVVVXV |
| 27 | F | 0.654 | # FLIFFVVFFLFLFLFXXFFVFILFFLLLLLLFFFFFFFFYFFFFFFFFFFFFFFF |
| | | | FFFFFIFLFLFFFFLFLFFFLILFFFFGLFFFFLLFLF-FFLLFFL |
| 28 | I | 0.863 | # ILIIIIIIIIIIILIIIIIIIILIIIIIIIIIIIIIIIIIIIIIIIIIIIIILII |
| | | | IIIIIIIIIIIIIILILIIIIIIYILIIIIIIIIVIII-IIIIIII |
| 29 | E | 0.918 | # EEEEEEEEEEEEEEEEEEEEEEEEEEEEEEEEEEEEEEEEEEEEEEEEEEEEEEE |
| | | | EEEEKEEEEEEEEEEEEEEEEEELEEEEEEEKLEEEEE-EEEEEEE |
| 30 | Y | 0.95 | # YYYYYYYYYYYYYYYYYYVYYYYYYYVYYYYYYYYVYYYVYYVYYYYYYYYYYYY |
| | | | YYYYYYYYYYYYYYYYYYYYYYYYYYYIYYYYIYYYYY-YYYYYYY |
| 31 | R | 0.899 | # RRRRRRRKKRRRRRRRRRRRRRRRRRRRRRRRRRRRRRRRRKRRKRRRKRRRRRR |
| | | | RRRRRRRRRRRRKRRRRRRRRRRKRRRRRRRRRRRRRRXRRRRRRR |
| 32 | K | 0.891 | # KKRRKKKKKKRKKKKKKKKKKKKRKKKKKKKKKKKKKKKKKKKKKKKKKKKRKKK |
| | | | KKKKRKKKRKKKKRKKKKKKKKKSKKKKKKKKKKKKKKKKKKKKKK |
| 33 | I | 0.927 | # IIIIIIIIIIIIIIIIIIILIIIIIIIIIIIIIIIIIIIIILIIIIIIIIIIIII |
| | | | IIIIIIIILIIIIIIILIIIIIIIIIITIIIIIIIIIIIIIIIIII |
| 34 | L | 0.827 | # LLLLLLLLLLLLLLLLLLLLRLLLLLLXLLLRLLKLLLLRLLLLLKLLLLLLLLI |
| | | | LLLLLVVLLLLLRRLLLLLLLLLLLLLLLLRLLLLLLLLLLLLLLL |
| 35 | R | 0.909 | # RRRRRRRRRRRRRRRRRRRRRRRKRRRRRRRRKRRRKRRKRRRRRRRRKRRRRRR |
| | | | RRRRKRRKRRRRRRRRRRRRRRRRRRRRRRRRRRRRRRRRRRRRRK |
| 36 | Q | 0.982 | # QQQQQQQQQQQQQQQQQQQQQQQQQQQQQQQQKQQQQQQQQQQQQQQQQQQQQQQ |
| | | | QQQQQQQQQQQQQQQQQQQQQQQQQQQQQQQQQQQQQQQQQQQQQQ |
| 37 | R | 0.863 | # RRRRRRRRRRRRRRRRRRRRNRRRRRRRRRRKKRRRRRRKRRRRRRRRKRRRRRR |
| | | | RRRRRRNRKRRRRKRRRRRRRRRRKRRKRRRRRRKRRRRRRRKRRR |
| 38 | K | 0.881 | # KKKRKKKKKEKKKKKKKKKKKKKKKKKKKKKQKKKKRKKKRKKKKKKKKKKKKKR |
| | | | KKKKKKKKKKKRKKKKKKKKKKKKKKKKXKKKKKKKKKKKKKKKKK |
| 39 | I | 0.964 | # IIIIIIIIIIIIIIIIIIIIIIIIIIIIIIIVIIIIIIIIIIIIIIIIIIIIIII |
| | | | IIIIIIIIIIIIIIIIIIIIIIIIIIIIIIIIIIIIIIIIIIIXII |

TABLE 6B+TL,47

| | | | |
|---|---|---|---|
| 40 | D | 0.945 | # DDDDDDDDDDDDDDDDDDDNEDDDDXDDDDDDDDDDDDDDDDDDDDDDDDDDDDD |
| | | | DDDDDDDDDDDDDDDDDDDDDDDDDDDDDDDDDDDDDDDDDDDDDD |
| 41 | R | 0.868 | # RSRRRRRKKRRRRRRRRRRRRRRRKRRRGRRRRKRRRRRRRRRRRRRRRRRKSRR |
| | | | RRRRRRRRRRRRRRRRRRKRKRRRRRRRRRRRRRRRRRRRRRRRRR |
| 42 | L | 0.901 | # LILLLLLLLLLLLLLLLLLLLLLLILLLIIILLLLLLLLLLLLLLLLLLLLLILL |
| | | | LLLLLLLLLLLLLLLLLLIILLLLILLLLLLLLLLLLLLLLLLLLL |
| 43 | I | 0.945 | # IIIIIIIIIIIIIIIIIIIIIIIIIILIIIIIIIIIIIIIIIIILIIIIIIIIII |
| | | | IIILIIIIIIILIIIIIIIIIIIIIIIIIIIIIIIIIIIIIIIIII |
| 44 | D | 0.813 | # DDDDDDDDDEDDDDENDDDDDDDDDDDDDDDDDDDEDEDDNDDEKDDDDDDTDDD |
| | | | DDDDDDDDDDDEDDDDDDDDNDDDDDDDDDDNDDDDDNKDDDDDDD |
| 45 | R | 0.927 | # RRRRRRRRRRRRRRRRRRRRRRRRRRRXRRRRRRRRRRRRRKRRRRRRRRKRRRR |
| | | | RRRRRRRRRRRRRRRRRRRRRRRRRRRRRRRKRRRRKRRRRRRRRR |
| 46 | I | 0.894 | # IIIIIIITTIIIIIIIIIILILIIIIIIIIIIIIIIIIIIIIIIIIIIIIIIIII |
| | | | LIIIIIIIIIIIIIIIIIIILLIIIIIIIIIIIIIIIIIIIIIILI |
| 47 | R | 0.588 | # RIRRRRRRRRARRRTIRRITIIRRIIXRRRRIIRTTRTSRRSRSATRRRRRIRAR |
| | | | ITRATRARRIIRRRRRRIRRVIRRIRRRRRSKRRRTRTSRRRRRRR |
| 48 | E | 0.932 | # EEEEEEEEEEEEEEEEEEEEEEEEEEEEEEEEEEEEEEEEEEEEEEEEEEEEEEE |
| | | | EEEEEEEEEEEDEEEEEEEEEEEEEGEEEEEEEEEEEEKEEDEEEE |
| 49 | R | 0.968 | # RRRRRRRRRRRRRRRRRRRRRRRRRRRRRRRRRRRRRRRRRRRRRRRRRRRRRRR |
| | | | RRRRRRRRRRRRRRRRRRRRRRRRRRRRKRRKRRRRRRRRRRRRRR |
| 50 | A | 0.914 | # AAAAAAAAAAAAAAATAAAAAAAAATEAAAAAAAAAAAAAAAAAAAAAAAAAAAA |
| | | | AAEAAAAAAAAAAAAAAAAAAAAAAAAAAEAAAAAAAAAAEAAAAA |
| 51 | E | 0.95 | # EEEEEEEEEEEEEEEEEEEEEEEEEEEEEEEEEEEEEEEEEEEEEEEEEEEEEEE |
| | | | EEEEEEEEEEEEEEEEEEEEEEEGEGEEEEEEXEEEEEEEEEEEEE |
| 52 | D | 0.982 | # DDDDDDDDDDDDDDDDDDDDDDDDDDDDDDDDDDDDDDDDDDDDDDDDDDDDDDD |
| | | | DDDDDDDDDDDDDDDDDDDDDDDADDDDDDDDDDDDDDDDDDDDDD |
| 53 | S | 0.982 | # SSSSSSSSSSSSSSSSSSSSSSSSSSSSSSSSSSSSSSSSSSSSSSSSSSSSSSS |
| | | | SSSSSSSSSSSSSSSSSSSSSSSSSSSSSNSSSSSSSSSSSSSSSS |
| 54 | G | 0.982 | # GGGGGGGGGGGGGGGGGGGGGGGGGGGGGGGGDGGGGGGGGGGGGGGGGGGGGGG |
| | | | GGGGGGGGGGGGGGGGGGGGGGGGGGGGGGGGGGGGGGGGGGGGGG |
| 55 | N | 1 | # NNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNN |
| 56 | E | 0.956 | # EEEEEEEEEEEDEEEEEEEEEEDEEEEEEEEEEEEEEEEEEEEEEEEEEEEEEEE |
| | | | EEEEEEEEEEEEEEEEEEEEEEEEEEEEEEEEEEEEEEEEEEEDEE |
| 57 | S | 0.982 | # SSSSSSGSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSS |
| 58 | E | 0.792 | # EEEEEDDEEEEEEDEEDDEEDEEEEDDEDDDEEGEDEDDDEDEDEEEEEEDEEEE |
| | | | DDEEEEEEEEDDDEEEEEEEEEEEEDEDEEEEEEEEEDEEEEDEEE |
| 59 | G | 0.938 | # GGGGGGGEEGGGGGGGGGGGGGGGGXGGGGGGGGGGGGGGGGGGEGGGGGGGGGG |
| | | | GGGGGGGGGGGGGGGGGGGGGGGGGGGGGGGGGGGGGGGGGGGGGG |
| 60 | D | 0.938 | # DDDDDDDDDDDDDDDDDDDDD-DDDDDDDDDDDDDDDDDDDDDDDDDDDDDDDDD |
| | | | DDDDDDDDDDDDDDDDDDDD--DDDDDDDDDDIDDDDDDDDDDDDD |
| 61 | Q | 0.863 | # QQQQQQQQQQQQQQQQQQQQQ-QQQQQQHHHQQQQQQQQQQQQQQQQQQQQQQQQ |
| | | | QQQQQQQQQQQQQQQQQQQQ--QQQEQEQQQQRQQQQQQQQQQQQQ |
| 62 | E | 0.865 | # EEEEEEEDDEEEDEEEEEEEE-EEEEEEEEEEEEEEEEEEEEEEEEEEEEEEEEE |
| | | | EEEEEDEEEEEEEEDEEEEE--EEEEEEEEEEKEDEEEEEEEEEEE |
| 63 | E | 0.914 | # EEEEEEEEEEEEEEEEEEEEEEEEEEEEEEEEEEEEEEEEEEEEEEEEEEEEEEE |
| | | | EEEEEEEEEEEEDEE-EEEEEEEDEAEEEEEKEEEEEEEEEEEEEE |
| 64 | L | 0.945 | # LLLLLLLLLLLLLLLLLLLLLILLLLLLLLLLLLLLLLLLLLLLLLLLLLLLLLL |
| | | | LLLLLLLLLLLLLLL-LLLLLVLLLLLLLLLLLLLLLLLLLLLLLL |
| 65 | S | 0.891 | # SASSSSSSSSASSSSSSSSSSSSSASSSSSSSSSSSSSSSSASSSSSSSSSSSSS |
| | | | SSSSSSASASSSSSS-SSSSSSSSSSSSSSSSSSASSSSSASSSSS |
| 66 | A | 0.805 | # EAAAAAATTMAAVAAAAAAAAAAAATKTTTTAAAAEAEAAAAAAAAAAAAAAAAT |
| | | | AAAAAAAAAAAAAAAAAAAAAATAAAAAAAAAAAAAAAAAAAAAAA |
| 67 | L | 0.956 | # LLLLLLLLLLLLLFLLLLLLLLLLLLLLLLLLLLLLLLLLLLLLLLLLLLLLLLL |
| | | | LLLLLFLLLLFLLLLLLLLLLLLLLLLLLLLLLLLLLLLLLLLLLL |
| 68 | V | 0.869 | # VVVVVVVVMVVVVMVVVVVVVVVMVVVVVVVMVVVMVMVVVVVVVVVVVVVVVVV |
| | | | VVVVVVVVVVVVMVVMVVVMVVVMMVVVVVVVVVVVVVVV-VVVVV |
| 69 | E | 0.874 | # EGDEEEEEEEEDGEEDEEEEEEEEEEEXEEEEEEEEEEEEEEEEEEEEEEEEEEE |
| | | | DEEEEEEEEEEEEEEEEEEEEEEEEEEEEEGEEEEEEEEEEEEDEE |
| 70 | M | 0.814 | # MMMMMMMMMRMMMMMMMMMRMMMMMMMMMMMMMIMMMMMMMMMMVMMMMMMMMMM |
| | | | MRMMRMMMMMGRMMMMMRMMMMVMMRMMMMMMMMMMRRMMRMRMMR |
| 71 | G | 0.936 | # GGGGGGGGGGGGEGEGGGGGGGGGGGGGGGGGGRGGGGGGGGGGRGGGGGGGGGG |
| | | | GGGGGGGGGGGGGGGGGGGGGGGGGGGGGGGGGGGGGGGGGGGGGG |
| 72 | H | 0.873 | # HHDHHHHHHHHHHHHHHHHHHHHHHHHHYYYHHHHHHHHHHHHHHHHHHHHHHHH |
| | | | HHHYHYRHHHYHHHYHHHHHHHHYHHHHHHHHHHHHHHHHHHHHHH |
| 73 | H | 0.66 | # LHRDHHHHHLHHHHHDDLHLDHHHHHHDHHHNHHHLDLHDHHHHHHHDHHHHHHD |
| | | | LHHHLHLDLHRLHDHLDLHHHHLHHLHHLHDDDHHHLLHHLHLHHD |
| 74 | A | 0.874 | # VAVAAAAAAAADDAAAAAAAAAAAAAAADDDAAAAAAAAAAGAAAAAAAAAAAAA |
| | | | AAAAAAAAAAAAAAAVAAAAAAAAAAAAAAAAAAAAAAAAAAAAAA |
| 75 | P | 0.964 | # PPHPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPLPPPPPPP |
| 76 | W | 0.868 | # WWLW$WW$$WWWWWWWWWWWWWWWWWWWWWWWW$WWWWWWW$WWKWWWWWWWWWW |
| | | | WWWWWWWWWWWWWWWWWWWWWWWWWWWWWWWWGWGWWWWWWWWWWW |
| 77 | D | 0.85 | # DDVDDDDDDDDDDDDDDDDDDDDDDDDDDDDDDDDDNDDDDDDDDDDDDDSDDDA |
| | | | DDDDDDDDDDDDDDDDDNDDDDDDHDNDDNVNDDDDDDDDDDDDDD |
| 78 | V | 0.766 | # VVVVVVVVVIVIVVVVVVVVVVVVVVVVIIIVVIVIVVIVVIVIIVVIVIIIVVV |
| | | | VIVVIVIIIIVVIIVAIIVIVIIIVIVVVVIVIIVVIVIVVIIVIV |
| 79 | D | 0.76 | # DDNANDDNNNDNNDNNNNDNNDDNDNNDNNNNDNDDDDNDNNDNDDDDDDNDDDN |
| | | | NNNNDNDDDDNNDDNDDDDNNDDDNDDDDDNNDDDDNDDDDDDDNN |
| 80 | D | 0.95 | # DDDDDDDDDDDDDDDDDDDDDDDDDDDDDDDDDDDDDDDDDNDDDDDDDDXDDDD |
| | | | DDDDDDDDDDDDDDDDDDDDDDDDDDDDDDDNDDDDDDDDDDDDDD |
| 81 | L | 0.906 | # LLLLLLLLLLLLLLLMLLLQLLMLLLLLLLLLLLLLLLLLLLLLLLLLLLLLLLL |
| | | | LLLLLLLLLGLLLLQLLLLLLLLQLLLLLLLLLLLLLLLLLLLLLL |
| | | | # $$$$$$$$$$$$$$$$$$$$$$$$$$$$$$$$$$$$$$$$$$$$$$$$$$$$$$$ |
| | | | $$$$$$$$$L$$$$$$$$$$$$$$$$$$$$$$$$$$$$$$$$$$$$ |

SAMPLE #: 154

| | | | |
|---|---|---|---|
| | Env | | |
| AA# | Cons. | entrophy | Raw Data |

| | | | |
|---|---|---|---|
| 1 | M | 1 | # MMMMMMMMMMMMMMMMMMMMMMMMMMMMMMMMMMMMMMMMMMMMMMMMMMMMMMMMMMMMMMMMMMMMMMMMMMMMMMMMMM |
| | | | MMMMMMMMMMMMMMMMMMMMMMMMMMMMMMMMMMMMMMMMMMMMMMMMMMMMMMMMMMMMMMMMMMMMMMMM |
| 2 | R | 0.788 | # RRRKKRRTTPTRRRRRKKRRRRRKRRRRRRRTRRKRRRKKKRRRRRKRRRRRRKRRRKRRRRKRRKRKKSRRRKKRRRRRRR |
| | | | RRRRRRRRRRRRRRRRRRRRKRRRRRRRRRKRRRRRRKRRRRRRRRRRKRRRRRRRRRRRRRRKRRRRRRTK |
| 3 | V | 0.81 | # AVAAVVVVVVVVVVVVVVVVVVVAVVVVVVAAVVVAAAVVAVVAVVAVAVAVVVVVAAVVVAAVVAAAAAVVAVVVAVVVVV |
| | | | AVVVVAVVVAAVAAVVVVVVVAVVVVVVVVVVVVAVVVAVAVVVVAVVAVVVVVVVVAVVVAVAVVVVVVVA |
| 4 | K | 0.757 | # KKKKKKKKKQMKKKTKKKTKKKKKKRKKKKKRKKKKKKKKKKXKRRKKKKKKMKMKKKKKKKKKKKRKKKKKKKTKKRKKRR |
| | | | RKKRKRKRRRKRKKKRRKKKKKKKKKKKKKKKKKMKMKKKKKKKKKKKKKTKKKKKKTKMKKKKKKKKKMKK |
| 5 | G | 0.776 | # EEGGEGGGEGEGGGGGEEEEGGEGGGEEGEGGEGGEEGGGGGGEGGGEGGEGEGGGGGEGGGGGGGEGGGGGGEGEEGEEEE |
| | | | EEEEEGEGEEEEEEEEGEEGGEGGGGGGEEEEGEEGEGGGGGGGGEGEEGEGGEGGEEEGGEEEGEGGGGEG |
| 6 | I | 0.722 | # IIITTIITTITIIIIIITIKIIIIIIITIIMTTIIITIIMIIVITITIITITIIIIITIIIIIIIIMIIIIITTIITIITTI |
| | | | TITTIIIITIIITIITTIIIIIIIIIIINKTTIIMIMTIITIISIIIITITIIIITIIIIIIIIIIIIIIIT |
| 7 | R | 0.827 | # RRRRKRRRRRRRKRRRRRR-WRRRRRRKRRRRRRRRRRRRRKRRRRRRRKRRRRMRRRRRRKRKRRRKRRRRRRRRRRRRRR |
| | | | RRRRRRRKRRRRRRRRRRRRRRKRRRRRYYRRKRRMRRRRRRRGRRRRRRRRRRRRRRRKRRRRRRRRRRRR |
| 8 | K | 0.728 | # RKKKRRKKKKKKMKKKKKK-RRRKKRKKKRKKKKKKKKKKKKKKKRKKKRKKRKRNKKKKKKRKRKRKKKKKKKKRRRRRRR |
| | | | RRRRKRRRRKRKRRRRMKKRKKKKKKKKQQKKKKKRKRKRKKKRRKKKKKKKKRKKKKKNKKKKKKRKKKKR |
| 9 | N | 0.828 | # NNNNNNNNNNNNNTNNNNN-NNNNNNNSNNNNNNNNNNNNNNNNNNNNNNSSNNNNNNNNNNNNNNNNNNNNNNNNNNNINN |
| | | | NNNNNNNSNNNKNNNNNHNNNNNNNSSSHHNNTNHNNNNNNNNNNSNNNSNNNNNNNNNNNNNNNNNNNNNN |
| 10 | Y | 0.638 | # WYCYWCYYYYYCCYYCYYYYCYYYYYYYFYYYYCCYYCCYCYCCYYYYYYYCYYY-CYYYCCYYYYWCYYY-YYYCWCCWW- |
| | | | WCWWWWWWWCCWWCWWC-YYYCYCYYYYLLCYYCCCCYCYYCCYYYYYYYYYYYYYYYYYYYYYCWTCYYYY |
| 11 | Q | 0.831 | # QQQQQQQRQQRQQQQQQQQQQQQQQQQQQQQQQQQQQQQQQQQQHQQRQQQQQQQ-QQQQQQQQQQQQQQQ-QQQQPQQQQ- |
| | | | QQQQQQQQQQQQQQQPQ-WQQQQQQQQQWWRQRQQQQQQQQQQQQQQQQQRQQQQQQQQQQQQHQQQQQQQQ |
| 12 | H | 0.665 | # HHRHRHHHHHHHYHHRHRHHLHHHHPRHRNHRHRRHHHHHRHHHHHHHHLHPPHHYRHHHHRHRHHDRHHHYHHHRDHRHHY |
| | | | HRHHRGRHHRHRHHHDH-LHHRHHHHHHRRHHHRHHHHHHHHHHRHHHHYHHHHHHHHHLHHHPHHHHHHHH |
| 13 | L | 0.812 | # LLLLLLLLLLLLLFLLLLLLLLLLLLLLHLLLLLLLLLLLLLLLY-LLWWLLLLLLLLLLLWLWLWLWWWWLLLLLLLLLLL |
| | | | LLLLWLLLWLLWLLLLLL-LLLLLWLLLWWWLLLLLLLLWLLLLLLLLLLLLLLLLLLLLFLLLLLWLLLLL |
| 14 | W | 0.974 | # WWWWWWWWWCWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWW-WWWWWW |
| 15 | R | 0.654 | # GIRRRRRTTRMRGKRRRRKRRRRRRRRKKTRRKRRKRRRKRRKRRRRRKRKTTRKKRKRRKRRRRRKRRRRRRRRTKTRKKR |
| | | | KRKKARRKKRRTKRKKRRRRKRRKRKKKKKKRRKRIKRRGRKKRRRRRRKRRKRRRRKRRRRRRKRGRKKIR |
| 16 | W | 0.86 | # WWWWWWWWWWWWWWWWWGWWWWWGWWGWWWWWWWWWWWWWWWWWWWWWGGWWWWWWWWWWGWWWGWWWWWWWWWWWWWWWWW |
| | | | WWWWWWWWWWWWWWWWWWGWWWWGWWWWWWWWWWGWWWWWWWWWWWWWWGWRWWWWRGWGWWRWWGWWWGWG |
| 17 | G | 0.977 | # GGGGGGGGGGGGSGGSGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 18 | T | 0.714 | # TTTTITTTTMTTMTTITTTTPITIIITTIIMTTTTTITTITIITTTTTTIIMTTMTTITTIITIIIIIITIMITTTTTTTTI |
| | | | TTTTTTTTTTITMITTTTTTITMIMTTTITIITTITTTTTTTTTITTTITTTITTITTTTTMTIIITMITTM |
| 19 | M | 0.805 | # MVMMMMMMMLMMMMMMMLMMLMMMMMLMMLMMMMMTLMMLMMLMMLMMLLMMMMMMMMMMLMMMLMMTMMMMMMMMMMMMMM |
| | | | MMMMMMMMMMMMMMMMMLLMMMMLMMMMMMMLMMLMMLMMMMMMMMMMTLMLMMMMMLMLMLLMMLMMMLMM |
| 20 | L | 0.964 | # LFLLLLLILLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLFLLLLLLLLLLLLLLLLLLLLLLLLLLLLLLLLLLLLLLLLLLLLLLLLLLLLLL |
| 21 | L | 0.927 | # LILLLLLLLLLLLLLLLLLLFLLLLLLLLLLLLLLLLLLLLLLLLLLLLLLLLLLLLLLLLLLLLLLLLLLLLLLLFLLLLL |
| | | | LLLLLLLLLLLLLLLFLLLLLLLLLLLLLLLLLLLFLLLLLLLLLLLLLLLLLLLLLLFLLGLLLLLLLLLL |
| 22 | G | 0.961 | # GWGKGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGMGGGGGGGGGG |
| 23 | M | 0.74 | # MLMMMMLIIMIMMIMMMMMMIIMMMLMMIIIMMMMIMMMIMMIMMMMMIMMMMMIMMMMIIMMMMMMMMMMMMMMMMMMMMM |
| | | | MMMMMMMIMMMMMMMMMMMMMMIIMIIIMIMTIMIMMMMLMMMIIIMMMIMIIIIMIMIIILIMMMLTIIMM |
| 24 | L | 0.896 | # LLLLLLLLLLLLLLLLLLLLLLFLLLLLLLLLLLLLLLLWLLLLLLLLLLLLLLLLLLLLLWLWLWWWWWWLLLLLLLLLLL |
| | | | LLLLLLLLLLLLLLLLLLLLLLLLLLLLLLLLLLLWLLLLLLLLLLLLLLLLLLLLLLLLLMLLLLLLLLLL |
| 25 | M | 0.942 | # MMLMMMMMMMMMMMMMMMMMMMMMRMMMMMMMMMMMMMMMMMMMMMMMMMMMMMMMMMMMMMMMMMMMMMMMMMMMMIMMMM |
| | | | MMMMMMMMMMMMMMMMMMMMMMMMMMMMMMLMMMMMMMMMMIMMMMMMMMMMMMMMMMMMMMMMMMMMMMMM |
| 26 | I | 0.987 | # IIIIIIIIIIIIIIIIIIIIIIIIIIIIIIIIIIIIIIIIIIIIIIIIIIIIIIIIIIIITIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 27 | C | 0.899 | # YCCCCCCCCCCCCCCCCSCCCCCCCCCCCCCCCCCCCCCSCCCCCCCCCCCCCCCCCCCCCCCCCCCCCCCCCCCSCCCYCY |
| | | | CCCCCCCCCCCCCCCCSCCCCCCCSCCCCCCCSCCCCCCCCCCCCCCCCCCCCYCCCCYCCCCCCCCCCCCC |
| 28 | S | 0.857 | # SNSSSSSSSSSSSSNSSSKSSSSSNNSSSNNSSSSSSSSSSSSSSSSNNNSSSSNKSSSSSNKSSSSSSSSSSSSSSSSSSS |
| | | | SSSSSRSSSSSSSSNSSSSSSSSSSSSSSSSSSSRSSSSSSSSSSSSSSSSSSSSSSSSSSSSSSSSSSSSS |
| 29 | A | 0.925 | # AAAAAAAAAAAAAAAAAVAAAAAAAAAAAAAAAAAAAAAAAAAAAAAAAAAAAAAAAAAAAAAAAAAAVAAAAAAAAAAAAA |
| | | | TAAAAATAAAACAAAAAAAAAAATAAAAAAAAAAAAAAAAAAAAAAAAAAAAAAAAAAAAAAAAAAATAAAA |
| 30 | A | 0.592 | # ATTATAAAAAEVTTAATAATTAETATTVTAKAATTTTAAELARAGTAAATG-TTRAATAAAATAVATAVAATAAASTTTAAA |
| | | | ATAAAAAATAASAAATATAAVATATTTTTTT-AATVATATAAAATEAAAVTTTAATAAATAAATAATAAAEA |
| 31 | E | 0.7 | # TEQGEEDNEDDEEEEEEGEEKEEEEEEEEEEEDEEEEEEEGEEEEDEEGEEEEEEEDEEEDEEEEGEEGEEEEEEEEEEEGE |
| | | | AEAAEEGGEEEEEEGEEEEDEGDGEEEEEEEEGETEEEAEEEEEEEEEEEGEEEGEDEGEEDEEDEEEEEEA |
| 32 | Q | 0.52 | # NQQQKHKNPKKQQNNKKNNKKNQRKKKQKKQNKKNNKQQQQQNNQQQQNEQQEQNQKKQQQRPRKGNREEKKQQNQQQQNNQ |
| | | | NQNNKNQQKQQKKQQQNNKQNNKQKNNNKKKKQNVQDKNKQQQQKEQKQKQQNQQKKQNKKQQKQKKKNQNN |
| 33 | L | 0.825 | # LLLLLLLFLLWLLLLLLLLLLLLLLLLLLLLLMLLLLLLLLLLLLMLLLLLLKLLLLLLLLMLMLLLMGLLLLLLLLLLLLG |
| | | | LLLLGLLLLLLGLLLLLSSLLLLLLLLLLLLLLSLLLLLLLLLLLLLLLLLLLLLMLLLLLLLLLTLLLLLL |
| 34 | W | 0.977 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWW$WWWWWWWWWWWWWWWWWWWWWWWWWWWWWW |
| 35 | V | 1 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 36 | T | 1 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 37 | V | 0.968 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVIVVVVVVVVVVVVVVVVVVVVIVVVVVVVVVVVVVVVVVVVVVVVVVVVVVVVVVVIVVVVVV |
| 38 | Y | 1 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 39 | Y | 1 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 40 | G | 0.987 | # GGGRGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 41 | V | 1 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 42 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 43 | V | 1 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 44 | W | 0.977 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWW$WWWWWWWWWWWWWWWWWWWWWWWWWWWWWW |
| 45 | K | 0.914 | # KKKKKRKRRRRKKKKKKKKKKKKKKKKKKKKKKKKKKKKKKKNKKKKKKKKKKKKKKKKKKKKKKKKKKKKKKKKKKKKKKK |
| | | | KKKKRKKKKKKRKKKKKKKKKKKKKKKKKKKKKKKKKKKKKKKKKKKKKKKKKKKKKKKKKKKKKRKKKKKR |
| 46 | E | 0.948 | # EEEEEEEEEEEEEEEEEEEEEEEEEEEEEEEEEEEEEEEEEEEEEEEEEEEEEEEEEEEEEDEDDEEDEEEEEEEEEEEEEE |
| | | | EEEEEEEEEEEEEEEEEEEEEEEEEEEEEEEEEEEEEEEEEEEEEEEEEEEEEEEEEEEEEEEEGEEEEEEE |
| 47 | A | 0.96 | # AAAAAAAAAAAAAAAAAAAAAAAAAAAAAAAAAAAAAAAAAAAAAAAAAAAAAAAAAAAAAAAAAAAAAAAATAAAAAAAAA |
| | | | AAAAAATAAAAAAAAAAAAAAAAAAAAAATAAAAAAAAAAAAAAAAAAAAAAATAAAAAAAAAAAAAAAAAA |
| 48 | T | 0.811 | # TNNTTTTTTATTTTTTTTTTTTTSTTTTDDTTTTTTTTTTTTTTTTTTTNTNTTTTTTTATTTTTVTTNTTTTTNTTTTTTT |
| | | | TTTTTTTNTTTTTSTTTTTTTTNTTATTTTTTTTTTTTTTTTTTTTTTTTTTTTTTTTTTNTATTTTTTNDN |
| 49 | T | 0.987 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTATTTTTTTTTTTTTTTTTTTTTTTTTTTTTTTTTTTTTTTTTTTTTTTTTTTTTTTTTTTTTTT |
| 50 | T | 0.987 | # TTTTTTTTTTTTTTTTTTTTTTTTTTTTTTTTTTTTTTTTTTTTTTTTTTTVTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 51 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 52 | F | 1 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 53 | C | 0.946 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCGGGCCCCCCCCGCCCCCCCCGCCCCCCCCCCCCCCCGCCCC |
| 54 | A | 1 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 55 | S | 1 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 56 | D | 0.933 | # DDNNDDDDDDDDDDDDDDDDDDDDDDDNDDDDDNDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDEDDDDDDDDDDDDDDDDNDNDDDDDDDDDDDDDDDD |
| 57 | A | 1 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 58 | K | 0.93 | # KKKKKRKKKKKKKKKKKKKKKKKKKKKKKKKRKKKKKKKKKKKKKKKKKKKKKKKKKKKKKKKKKKKKKKKKKKKKKKKKKK |
| | | | KKKKQKKKKKKKKKKKKKKKKKKKKKKKKPKKKKKKKRKKKKKKKKKKKKEKKKKKKKKKKKKKKKKKKKKK |
| 59 | A | 0.929 | # AAAGAAAAAAAAAAAAAAAAAAAAAAAAAAAAAAAGAAAAAVAASTAAAAAAAAAAAAAAAAAAAAAAAAAAAAAAAAAAAA |
| | | | AAAAAAAAAAAAAAAATAAAAAAAAAAAAAAAAAAAAAAAAAAAAAAAAAAAAAAAAAAAAAAAAAAAAAAA |
| 60 | Y | 0.943 | # YYYYYYYYYYYYYYYYYYYYYYYYYYYYYYYYYYYYYYYYYYYYYYYYYYYYYYYYYYYYYQYQYYYQYYYYYYYYYYYYYY |
| | | | YYYYYYYYYYYMYYYYYYYYYYYYYYYYYYSYYYYYYYYYYYYYYYYYYYYYYYYYYYYYYYYYYYYYYYYY |
| 61 | D | 0.701 | # DDDDDDDDDDDDSDDDDDDDVDDDDNDDDDDADDEDDDDDDDDDDEDADDSVDEDDDDDDDNDNDDDNDDDDDDDVVDDDDD |
| | | | DVDDNEDDEVVNDDVVDSDSDDDDDDDDEDDDDDKSKDDDDNDDDDDDDDDDDEDDEDDDDDDDDDDDDDGD |
| 62 | T | 0.808 | # TTTTKTTETTTTTTTTKTTTQTTTTTTTTTTTTTTKKTTTTTTTTTTTTTTKTTTTTTTTKRTRTTKRTTTTTTTPATTTTT |
| | | | TTTTTTPTTTTTTTTATKTTTTTTTTTTETTTKTATTTTTTTTTTTTTTTTTTTTTTTTTTTTTTTTTTTTT |
| 63 | E | 0.977 | # EEEEEEEEEEEEEEEEEEEEEEEEEEEEEEEEEEEEEEEEEEEGEEEEEEEEEEEEEEEEEEEEEEEEEEEEEEEEEEEEEE |
| | | | EEEEEEEEEEEEEEEEEEEEEEEEEEEEEEEEEEEEEEEEEEGEEEEEEEEEEEEEEEEEEEEEEEEEEEEE |
| 64 | V | 0.722 | # VAVVVVVVVMAVKVVVVVVVAVVVVAVVVVVVAVVAVVVVVVVVVVVVVAVAVAVVVKAVVVVVVVVVAAVVVVVKKKVVVV |
| | | | VKVVAKVMKKKVVVKKVVAVVVIVRAAAVVVVVVAAVVAVVVVVVVVVVVVVVVVVVVVVMVVAVVAVVVVA |
| 65 | H | 0.987 | # HHHHHHHHHHHHHHHHHHHHHHHHHHHHHHHHHHHHHHHHHHHHHHHHHHHHHHHHHHHHHHHHHHHHHHHHHHHHHHHHHH |
| | | | HHHHHHHHHHHHHHHHHHHHHHHHHHHHHHHHHHHHHHHHHHHHHHHHHHHHHHHHHHHHHHHHHHHHHHHH |
| 66 | N | 1 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNN |
| 67 | V | 0.92 | # VVVVVVVVVVVVIVVVVVVVVVVVVVVVVVVVVVVVVVVVVVVVMVVVVVVIVVVVVVVVVIVIVVVIVVVVVVVVVVVVVV |
| | | | VVVVVVVVVVVVVVVVVVVIVVVVVVVVVVVVVVVVVVVVVVVIVVVVVVVVVVVVVVVVVVVIVVVVVVVV |
| 68 | W | 0.987 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 69 | A | 1 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 70 | T | 0.974 | # TTTTTTTTTTTTTTTTTTTTTTTTTTTTTTTTTTTTTTTTTTTTTTTTTTTTTTTTTTTTTTITTTTTTTTTTTTTTTTTTT |
| | | | TTTTTTTTTTTTTTTTTTTTTTTTTTTTTTTTTTTTKTTTTTTTTTTTTTTTTTTTTTTTTTTTTTTTTTTT |
| 71 | H | 0.974 | # HHHHHHHHHHHHHHHHHHHHHHHHHHHHHHHHHHHHHHHHHHHHHHHHHHHHHHHHHHHHHHHHHHHHHHHHHHHHHHHHHH |
| | | | HHHHHHHHHHHHHHHHHYHHHHHHHHHHHHHHHHHHHHHQHHHHHHHHHHHHHHHHHHHHHHHHHHHHHHHH |
| 72 | A | 1 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 73 | C | 0.987 | # CCCCCCCCCCCCCCCCCCCCCCCCCCCCCCCCCCCCCCCCCCCCCCCCCCCCCCCCCCCCCCCCCCSCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 74 | V | 0.987 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 75 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 76 | T | 0.939 | # TTTTTTTTTTTTTTTTTTTTTTTTTTTTTTTTTTTTTTTTTTTTTTTTTTTTTTTTTTTTTITITITIIIITTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 77 | D | 0.977 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDNDDDDDDDDDDDDDDDDDDDDDDDDDDDDDDDDD |
| 78 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 79 | N | 0.83 | # DNNNNNNNSNSSNSNNDNNNNNNNNSNNNNNNNNNNNNDNNNNNNNNDNNNNNNNDNNNNNNNNNNNNNNNNNNNNSSNNNN |
| | | | NNNSNNNNDNNNNNNSNSNSNNNNNNNNNNNNDSNNNNNNNNNNNNSNNNNNNNNNDNNSNNNNNNNNNNNS |
| 80 | P | 0.987 | # PPPPPPPPPPPPPPPPPPPPPPPPPPSPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 81 | Q | 0.952 | # QQQQQQQQQQQQQQQQQQQQQQRQQQQQRQQQQQQQQQQQQQQQQQQQQQQQQQQQQQQQQQQQQQQQQQQQQQQQQQQQQQ |
| | | | QQQQQQQQQQQQQQQQQQQQQQQQQQQQQKQQQQQQQQQQQQQQQQQQQQQQQQQQQQQQQQQQQQQQQQQQ |
| 82 | E | 0.974 | # EEEEEAEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 83 | V | 0.757 | # IVLVIVVVIVIIVMVVVVVVIIVVVVVVIVVVVVVQVIIVVLIVVVVVIVVIVVVVVVVVMVIVVVVVVVVVVVVVVVVVVV |
| | | | VVVVVVVVVVVVVVVVVIVIVVLIVLLLIVVILVVVVVVVVVVVIVVVVVVVVVVVVVIMVVVVVVVVVIMV |
| 84 | V | 0.475 | # NEVRLAVEPVAVPKVPVVEVVVVVVVRAEAAVEVVFEIIAVVKVVVVEVVEIEKVAVVVVLVAVERVVVVFVVVVVVVVVVM |
| | | | VVELVVVVVVIVVVVVTPEVVVVALKKKVVVAPVNILVVEVVVVVVAGVVVVVVVGVKVIVLVDRVEVVLVV |
| 85 | L | 0.908 | # LLLLLLLLLLLLLLLLLMLLLLMMLLLLLLLLLMLLLLLLLLLLLLLLMLLLLLLLLLLLLLLLLLLLLLLLLLLLLLLLLL |
| | | | LLLLMLLLSLLMLLLLLLLMLLMLLLLLLLLLLLLLLLLLLLLLLLLLLLLLLLLLLLLLLLLLLLLMLLLL |
| 86 | E | 0.543 | # PVKEEAKIEGEEEEGIEGKVEEGEEEEEKGEGGGG-KEEVEEEEGGGGQAEAEEEEEGVEEEGEKVAGEEEGEGGEEGGGGE |
| | | | EEEEGGEEGGEGEEEEGKKEEKGGEEEEAVEEEETGEGEVQKKGEEEEEEVGGGGEEEEKEEEEEGVGEEGK |
| 87 | N | 0.987 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNDNNNNNNNNNNNNNNNNNNNNNNNNNNNNNNNNNNNNNNNNNNNNNNNN |
| 88 | V | 0.987 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVAVVVVVVVVVVVVVVVVVVVVVVVVVVVVVVVVVVVVVVVVVVVVVVVVVVVVVVVVVVVVVVV |
| 89 | T | 1 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 90 | E | 0.987 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 91 | N | 0.76 | # NENNENNNKNHNNNNDDNNNNNEDNNDEENNNNNNNNEDNNNNNNNNNNYNNNNNNNNNKNNKNEDNNNNNNNYNNNNNNNN |
| | | | NNNNNNNNNNNKNNNNNNNENNNNNNNNDNNDNNNNNNNNNNNNNNNNNHKNNNNNNNDNNNNNENNNNDNN |
| 92 | F | 0.974 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFSFFFFFFFFFFFFFFFFFFFFFFFFFFFFFFFFFFFFFFFFFF |
| 93 | N | 0.943 | # NNNNNNNNNNNNNNNNNNNNNNNNNNNNNNNDNNNNNNNNNNNNNNNNNNNNNNNNNNNNNNNNNNNNNNNNNNNNNNNNNN |
| | | | NNNNNNNNNNNNNNNNNSDNNNNNNNNNNHNNNNNNNNNNNNNNNNNNNNNNNNNNNNNNNNNNDNNNNNNN |
| 94 | M | 0.933 | # MMMMMMMMMMMMMMMMMMMMMMIMMMMVMMMMMMMMMMMMMAMMMMMMMMMMMMMMMMMMAMMMMMMMMMMMMMMMMMMMMM |
| | | | MMMMMMMMMMMMMMMMMMMMMMMMMMMMMMMMMMMMMMMMMMMMMMMMMMMMMMIMMMMMMMMMMMMMMMIM |
| 95 | W | 0.946 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWGWWWGWWWWWWWWWWWWWWWWWWWWWWW$WWWWWWWWGWWWWWWWWWWWWWWWWWWWWW |
| 96 | K | 0.952 | # KKKKKKKKKKKKKKKKKKKKKENKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKTKKKKKKKKKKKKKKKKKKKKKKKKKKKKKKKKKKKKKKKKKK |
| 97 | N | 0.974 | # NNNNNNNNNNNNNNNNNNNNNNNNNNNNNNNNNNNNNNNNNNNNNNNNNSNNNNNNNNNNNNNNNNNNNNNNNNNNNNNNNN |
| | | | NNDNNNNNNNNNNNNNNNNNNNNNNNNNNNNNNNNNNNNNNNNNNNNNNNNNNNNNNNNNNNNNNNNNNNNN |
| 98 | N | 0.839 | # NNNNNNNNDNNNNNNNNDNDNDSSNNNNNNNNSNNDNDDNNNNNNNSNKNDDNNNNNNNNNNNNNSNNNNNNNNNNNNNNNN |
| | | | NNNNNNNNNNNNNNNNNNNNNNDNNNNNEDNNNNNNNNNNNNNNNNNNNNNNNNNNNNNNNDNNNNNNNNNN |
| 99 | M | 0.987 | # MMMMMMMMMMMMMMMMMMMMMMMMMMMMMMMMMMMMMMMMMMMMMMMMMMMMMMMMMMMMMMMMMMMMMMMMMMMMMMMMMM |
| | | | MMMMMMMMMMMMMMMMMMMMMMMMMMMMMMMMMMMMMMMMMMMMMMMMMMMTMMMMMMMMMMMMMMMMMMMM |
| 100 | V | 0.977 | # VVVVVVVVVVVVVVVVVVVVVVVVVAVVVVVVVVVVVVVVVVVVVVVVVVVVVVVVVVVVVVVVVVVVVVVVVVVVVVVVVV |
| | | | VVVVVVVVVVVVVVVVAVVVVVVVVVVVVVVVVVVVVVVVVVVVVVVVVVVVVVVVVVVVVVVVVVVVVVVV |
| 101 | E | 0.841 | # EEEEEEEEEEEKEEEEEEDEEEEEEEEEEEEEEEEENEEEEEEEEEEEEEEEEEEEEEEEEEEEEEDEEEEEEDEEDENEEE |
| | | | EEEEDEDEEDDDEDDDDEEEEEEDEEEEEEEEEEEEEEEEEKEEEEEEEEEEDEEEEEEEEEEEEDEEEEED |
| 102 | Q | 0.987 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQ$QQQQQQQQQQQQQQQQQQQQ |
| 103 | M | 1 | # MMMMMMMMMMMMMMMMMMMMMMMMMMMMMMMMMMMMMMMMMMMMMMMMMMMMMMMMMMMMMMMMMMMMMMMMMMMMMMMMMM |
| | | | MMMMMMMMMMMMMMMMMMMMMMMMMMMMMMMMMMMMMMMMMMMMMMMMMMMMMMMMMMMMMMMMMMMMMMMM |
| 104 | H | 0.897 | # HHHHHHHHHHHHHHHHHQHHHHHHHHHHHQHQHQHHHHHHHHHQHQHHHHHHHQHHHHHHHHQHHHHHHHHHHHHHHHHHHH |
| | | | HHHHHHHHHHHHHHHHQHHHHHHHHHHHHHHHHHHHHQHHHHHHHHHHHQHHHHHHHHQHHHHHHHHHHHHL |
| 105 | E | 0.903 | # EEKEEEEEEEEEEEEEEEEEQEEEEEEEEEEEEEEEEEEEEEEEEEEEEEDEEEEEEEEEEEEEEAEEEEEEEEEEEEEEEE |
| | | | EEEEEEEEEEEEEEEEEEEEEEEEEEEETEEEEEEEEEEEEKEEEGEEEEEEDEEEEEEEEEEEEEEEEEEE |
| 106 | D | 0.977 | # DDDDDDDDDDDDDDDDDDDDDDDDDDDDDDDDDDDDDDDDDDDDDDDDDDDDDDDDDDNDDDDDDDDDDDDDDDDDDDDDDD |
| | | | DDDDDDDDDDDDDDDDDDDDDDDDDDDDDDDDDDDDDDDDDNDDDDDDDDDDDDDDDDDDDDDDDDDDDDDD |
| 107 | I | 0.968 | # IIIVIIIIIIIIIIIIIIIIIIIIIIIIIIIIIIIIIIIIIIIIIIIIIIIIIIIIIIIIIIIIIIIIIIIIIIIIIIIVII |
| | | | IIIIIIIIIIIIIIIIIIIIIIIIIIIIIIIVIIIIIIIIIIIIIIIIIIIIIIIIIIIIIIIIIIIIIIII |
| 108 | I | 0.987 | # IIVIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 109 | S | 0.974 | # SSDSSSSSSSSSSSSSSSSSSSSSSSSSSSSSSSSSSSSSSSSSSSSSSSSSSSNSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 110 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 111 | W | 0.987 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 112 | D | 0.977 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DEDDDDDDDDDDDDDDDDDDDDDDDDDDDDDDDDDDDDDDDDDDDDEDDDDDDDDDDDDDDDDDDDDDDDDD |
| 113 | Q | 0.892 | # QQQQQQQQQQQQQQQQQQQQEQQQQQEQQQQQQEEQQQQQQQQEQQQQQQQQQEQQQQQQQQQQQQEQQQQQQQQQQQQQQQ |
| | | | QQQQQEQQQQQQQKQQEQQEQQQQQQQQEQQQQQQQQQQQQQQQQQQQQQQQQEQQQQQQQQQQQEQQQQQQ |
| 114 | S | 0.987 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSRSSSSSSSSSSSSSSSSSSSSSSSSSSSSSSSSSSSSS |
| 115 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 116 | K | 0.987 | # KKKKKKKKKKKKKKKKKKKKKKKKKKKKKKKKKKKKKKKKKKKKKKKKKKKKKKKKKKKKKKKKKKKKKKKKKKKKQKKKKK |
| | | | KKKKKKKKKKKKKKKKKKKKKKKKKKKKKKKKKKKKKKKKKKKKKKKKKKKKKKKKKKKKKKKKKKKKKKKK |
| 117 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 118 | C | 0.987 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCRCCCCCCCCCC |
| 119 | V | 1 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 120 | K | 0.904 | # KKKKQKKKKKKKKKKKKKKKKKKKKKKKRKKKKKKKKKKKKRKKKKKKKKKKKDKKKKKKKKKKKKKKKKKKKKKKKKKKKK |
| | | | KKKKKKEKKKKKKKKKEKKKKKKKKKKKKKKKKKKKKKKKKKKKKKKKKKEKKKKKKKKKRKKKKKKKKKKR |
| 121 | L | 0.96 | # LLLLLLLLLLLLLLLLLLLLLLLLLLLLLLLLLLLLLLLLILLLLLLLLLLLLLLILLLLLLLLLLLLILLLLLLLLLLLLL |
| | | | LLLLLLLLLLLLLLLLLLLLLLLLLLLLLLLLLLLLLLLLLLLLLLLLLLLLLLLLLLLILLLLLLLLLLLL |
| 122 | T | 0.987 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTPTTTTTTTTTTTTTTTTTTTTTTTTTTTTTTTTTTTTTTTT |
| 123 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 124 | L | 0.974 | # LLLLLLLLLLLLLLLLLLLLLLLLLLLLLLLLLLLLLLLLLLLLLLLLLFLLLILLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 125 | C | 0.987 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCYCCCCCCCCCCCCCCCCCCCCCCCCCCCCCCCCCCCCCCCCCCCCCCCCCCCCCCCCCCCCCCCCC |
| 126 | V | 0.987 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVDVVVV |
| 127 | T | 0.927 | # TTTTTTTSTSTTTTTTTTTSTTTTTTTTTTTTTTPTTTTTASTTTTTTTTTTTTTTTTTTTTTTTTTTTTTTTTTTTTTTTT |
| | | | TTTTTTTTTTTTTTTTTTTTTTTTTTTTTSTTTTTTTTTTTTTTTTTTTTTTTTTTTTTTTTTTTTTTTTTT |
| 128 | L | 0.977 | # LLMLLLLLLLLLLLLLLLLLLLLLLLLLLLLLLLLLLLLLLLLLLLLLLLLLLLLLLLLLLLLLLLLLMLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 129 | N | 0.716 | # NNDDNNNNNNNNNNNNNKNKKNNNNHNNNNNDNNNNNDDNNNHNNKNDNNHNNNNKINHNNNNNKNMTNNKDHDNNNNNNNN |
| | | | INENNNNNNNNNNNNNKNNNNNNNNNNNNKNNNNNNNNNNNNNNHNNNNNNDNDNNNNNNNKNKNNNNNHNK |
| 130 | C | 0.987 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCRCCCCCCCCCCCCCCCCCCCCCCCC |
| 131 | T | 0.722 | # TTTTTTTTTTTTTTTTTTTTTTTTTTTTITTTTTTTTTTTSITTTTSFQSTTSTTTTTTIYSSSTSASSSTTTDTTTTTTTS |
| | | | TTTTTTTTTTTITTTT-TTTTTHTTSSSTTTTTTTITTTTTTTTITTTTKTITTTIITTTTTTTTTTITTT- |
| 132 | D | 0.774 | # DNDDDDDDDDDN-DNDDDDDNDN-NDNDDDDDDNNNDDDDNDDNDDNDS-DDDDDDNDDDDDDD-NNDNNDDDDDDDDDDDD |
| | | | DDDDDDDNDDDDDDDD-DDTDDDDDDDDDDDDDNDDDDDDDDDNDDDDDDDDDDDDDDDDDDDNDNDNNDN- |
| 133 | L | 0.317 | # LWYVEVYAAYLW-ELLLYYLVLY-ALLYVYLVNWWVELLVLVAWNLVVL-LYDCAVVLLWYVWV-VVVLLLVLVLNNKEVVT |
| | | | VKVVYEVLEEKYDNNN-LVELYFFVLLLLLNLFWK-ALLLLYYLYLEDDVLYDDLLLLELLLDDVLLVVLY- |
| 134 | L | 0.255 | # -MKKLKSKTVEK-VKKKNGK----GGEVKVR-LMK-LRS--E-KLQDNK-KVILNT-RRGVKPK-K-KDR--K-RLLLLL-W |
| | | | SLLSWVPRLLLVLLLL-KSLRL--T---KKLVSKLKNGNRTLLRL-LLL-R-FLRKRRLRRQLLW-RK-KV- |
| 135 | N | 0.828 | # NNNNNNNNNNNNNKNNNNNNNNNNNNNTNNNNNANNNTNNNNNNNNNNNNNNNNNNNNNNNNNNNNNNKNNNNNNNNNNKNN |
| | | | NNNNDSGNNNNNNNNNNNNLNNNNNNNNNNRNNNSNNTNNNNNNNNNNNNNNNNNNNNNTNNNNNNNKNNKN |
| 136 | A | 0.305 | # ANAVVAATNATSVAAAAADDTVSLIAANSTATVTNTANDADNCNGTNANAGKNASCNDTDTGRGVEVGTNDVATADDIGGGD |
| | | | SDDSTYNGGDNDVDDGDADNVAAASTTTEDNADVTIGNNTANDDATVAAAANNAAADAANDANTAITTVGID |
| 137 | T | 0.787 | # TTTSTTTTSTTTTTTTTTTTTTTTTTTTTTSTTNTTTCTTTTTTTTDTTTTTTTTTTTTTTTTTTTTTNTTTTTTTTSTTTT |
| | | | STTTNAETTTITTDTTTTTCTTTTTTTTTTDTTTNTTTTTTTTTTTTTTTTDTTTTTTTNTTNTTTTNTTDT |
| 138 | N | 0.294 | # EL-DFN-NS-ISIS--N-NNNDSP-NNT-SISYNNDFTNNN-K-INT-NY-TNDDGRNNSSSTSKSNDNNND---KSSRSS- |
| | | | GSSSTNTNYGSNY-SRTSMIN--SGSSSKNTNNYGD-NTNY--S-T--V-NTNN-----T--I-SKNSNNTR |
| 139 | N | 0.852 | # NNNNNNNNKNNNNNNNNNNNNNNNN-NNNNNRNGNNNSNNNNNNNNNNNNNNNLNNNNNNNNNNNNNNNNNNNNNNNENNNN |
| | | | NNNNNNNNNNNNNKNNNNNTNNNNNNNNNNNNNNTNNNNNNNNNNN-NNNNNNNNNNNN-NN-NNNNNNNNN |
| 140 | S | 0.307 | # NTTTSSTSTTSSNTTTNSNSNSDVN-SATSINSSNGTNCATTCSATPSTSAITAACSNSATSSSVNMNTSNSTSTSSKIAVA |
| | | | ISIIDESIVSSNISSSDTANNTAFVGGGSSDANSSTVWTNGTTATS-TATSSISTTTTT-TN-TTTNIEGAN |
| 141 | T | 0.57 | # ASTIRTTSNTSSSTSTSSSSTSTTS-STTANAGINTTSTTNTTTTTTTTSTTTCTTSSSTTSSSTSTSTSSTTTSSS-STTT |
| | | | TSTTTTTTTSSSTTSSSSTDSTTTTEEESSTTSSGTTKTSSTTTTS-TTTSSSSTTTTT-TS-TTTSTTTTT |
| 142 | N | 0.502 | # NRNNNNMNNSR--A----NSNR-N---KN-NN-TNNNANTNNNNNISNNNN--NNNDIINNNNNNNNNNNNNN-----NN-- |
| | | | ----N------N-N--NNA----NN---DSNN--T---ENSSSSNTSNNNSSSSSNSSNTNNSNNNNNNKVN |
| 143 | M | 0.45 | # DMG-MEMKGMIE-MMMMMMMMMTMSMRTMMNMVIIMMERMMIMMIMSMQISMMMMMMMMMMMMMMMMMKMMMMIMVEMVSIG |
| | | | SVSSMNVQTVQM-VVEMIMDMMMM-RRRMMGLMMMGMERIEMMMMMKMMMMMMMMMMMMGMMMMMMISMMMM |
| 144 | E | 0.531 | # -E--EEEAEEEE-EE--EEEE-K-SEEEEE-EEEEEEEAEEEEEDEGIGVEEIEEEKEKEMGEGEDEEMEGEERGQQEEEEE |
| | | | GQGEEEEEEEEEEEQQEEEQ--EV-EEEGEEEEEPGEIGKEKEEEEMEEEEEDEEEEEELEGEEEEK-E-EE |
| 145 | K | 0.357 | # ING-KEK--RKKTRGKKKGKAKERGK-RQR-KKIIEKKRRKKKERKEEEDRKEAAK-G-KGEKEEK-EKKEKKKGTKIEKKK |
| | | | KKKKETDRKK-TKVKKKRKKRRG--RRRNKTKKRE-NKKGRGGGRKKMRRRKEKRPRKRREIRTKKGEEREP |
| 146 | G | 0.781 | # QGEIGGGGGGGGGGGGGGGGGGTEEGGGGGSGGEEGGEEGGGGGGGGGGPGGGGGGGGGGGGGANGGGDGGGGGGGGGGGGG |
| | | | GGGGRGGEGGGGGGGGGTGGGGGKNGGGEGGGGGGGGGGGGGGGGGGGGGGGGGGGGGGGGGGGGGGGGGGG |
| 147 | E | 0.895 | # NEEVEEEEEEEEDEEEEEEEEEGEEEEEEEEEEEEEEEEEEEEEEEEEEEEEEEEEEEEEEEEEEEGELDEEEEEEEEEEEE |
| | | | EEEEEEEEEEEEEEEEEEEEEEEEEEEEEEEEEEEEEEEEEKEEEEEEEEEEEEEEEEEEDEEEEEEEEEEE |
| 148 | I | 0.761 | # MVIMMIIIIIIIMIIMIIIIIIIMIIMIIIMLIIIIIMIIMIIIIILIMMLIIMIMIMIMMMIMIMIMMMMIIIIIIIIIII |
| | | | IIIIMIIIIIIMIIIIIMIMIIMMMIIIIIIIIMIVIIMMIIIMIIIIIIIIIIIIIIIMMIIIMIMIMIII |
| 149 | K | 0.92 | # KKKKKKKKKKKKKKKKKQKKKKKTKKKKKKKKKKKKKKKKKKKKKKKKKKKKKKKKKKKKKKKKKKKKKRKKKKKKKKKKKK |
| | | | KKKKKKKKGKKKKKKKKKKKKKKKKKKKKKKKKKKKKKRKRKKTKKKKKKKKKKKKKKKKKKKKKKKKKKKK |
| 150 | N | 0.943 | # NNNNNNNNNNNNNNNNNNNNNNKNNNNKNNNNNNNNNNNNNNNNNNNNNNNNNRNNNNNNNNNNNNNNNNNNNNNNNNNNNN |
| | | | NNNNNNNNDNNNNNNNNNNNNKNNNNNNNNNNNNNNNNNNNNNNNNNNNNNNNNNNNNNNNNNNNNNNNNNN |
| 151 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 152 | S | 1 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 153 | F | 0.961 | # FFFFFFFFFCFFFFFFFFFFFFFFFFFFFFFFFFFFFFFFFFFFFFFFFFFFFFFFFFFFFFFFFFFFFFFFFFFFFFFFFF |
| | | | FFFFFSFFFFFFFFIFFFFFFFFFFFFFFFFFFFFFFFFFFFFFFFFFFFFFFFFFFFFFFFFFFFFFFFFF |
| 154 | N | 0.863 | # NNNNNNNNNNNNNNNNNKNNNNNKKNNNNKNNN--KNNNNNNNKNKNNNNNKNNNNNNNNNNNNNNNNNNNNNNNNNNNNNN |
| | | | NNNNNSNNNNNNNNNNNNNNNNNNNNNNNNNNNNNNQNNNNNNNNNNNNNNNNNNNNNNNNNNNSYNNNNNN |
| 155 | I | 0.71 | # IIITIIIIIVIVIVIIIVIIVTIIVYVTIIITI--TIIIIVIIVIIIIIIITIIIVVIVIIVIVIVIVVVVIIIIIIIIIIV |
| | | | IIIIIVTAIIIIIIIIVIIIIVIIIIIIVIIIIVIIVIIIVIIIIIIIIIIIIIIIIIVIIAIIIIIIVVVF |
| 156 | T | 0.864 | # TTSTTTTTTTDTTTTTTTTSTTTTTTTTTTTTTTITTTTTTTTTTTTSTTTSTTTTTTTTTTTTTTTTTTTTTTTTTTTTTT |
| | | | TTTTTPTTTTTTTTTTTTTTTTTTTTTTSGTTTTTTTTTTTTTTTTTTTTTTTTTTTT-TTTNTTTATTTTT |
| 157 | T | 0.853 | # ETTTTTTTTTTTATTTTTRTTTTTTTTTTTTTTTTTTTTATTSTTTTTTTTTSTTTTTTTATTTTTTTTTTTTTTTTSTTTA |
| | | | TTTTTTTTTTTTTTTTTTTATTTTTTTTTTTKTTTTTTTTTTTTTTTTTTSTTTTTTT-TTTTTTTTTTKTT |
| 158 | S | 0.442 | # GNSSSRNNSSDSGSSRSNSSNRSSNTSSNSNSNSNSSNNSGSNSNSRDSSSGDNRNSSNSSNSNNNNNSTNSAS-DGGGTTG |
| | | | AGAVSGGGAGGSEGGGSDTGSRSNNSSSGSSNESSSSSSSIKRSSSSSSSSSSRSRSS-TSSRNSSSSLNAS |
| 159 | I | 0.697 | # IMIIIIIMMMMIIIIIIIIIIIVIIIIVIIIIMIIMMIIIIIIIIIIIIMIILILIIIIIIIIIIIIIIIIIIISIIIILII |
| | | | VIVVIIIIIIIVVIIILLLIIIIIIMMMAIIMVIVRRIIIIIIIIIIIIIIIITIIIILLIIIRIIIIIIIM |
| 160 | R | 0.675 | # KKRKRRRRRRGKKRKRRRRRRKKKRRKRRRRRRGGKKRRRRRRKKSGRRRRKKGRRRRRRRGRGRGKGRKRRRRIRRRRRRR |
| | | | RRRRRKRRRRKRQRKRRRRRRRRRRTTTPRRRRKRRRRRRREGRRRRRRRRRRRRRRRGRRGRRRRGRRIRR |
| 161 | D | 0.676 | # NDNDDNGDDDANDDTDDDDGDDDDDDNDGDDDDNNDDDDDNDDNDDEDDDDDDNDNNDDDDNDNNDNDNDDDDRGGNGDDDD |
| | | | DGDDDDGDDGGDDDGGNDDNDDDDDDDDGDDDDNDDDDDDNNNSDDDDDDDDDTDDDDDDNDDGDNDDNDDT |
| 162 | K | 0.844 | # RKKRKKKKKKKKKKKKKKKKKKRKKKKKRKKKKEKKKKKKKEKKKKKKKKKRKKKQKKKKKKKKKKKKKKKKKKTRKKRKKK |
| | | | KRKKKKRKKRRKKRRKKKKKKKKKKKKKKKKKKKKKKKKKKKKKKKKKKEKKKRKKKKKKKKKKKKKKKKKK |
| 163 | V | 0.562 | # RVVMRVVVVVAVVMVVVVVVRGFMTVQVMVMMRIIMVIIMLVWMMVVLVMVAVVVVVMVVMVVVVMRVMVVGVVKVVMVVVV |
| | | | VVVVVVVMVVVVMFVVVVVVVVMVIMMMVVVVIVVVTIVMIVVVMVVVVVVMVVMPVVKMMVVMVVMVRVVV |
| 164 | Q | 0.648 | # KQQQHQQQQQQQKQ-QQQKQKQTQQQQQQQQQKKKQQQQQQKQQQQQQQTQQQKQRRQQQQKQKQKEKQKQQRQV--Q-QQQ |
| | | | Q-QQQQ-QQ--RKK--QKQRKQQQKQQQQQQEQQQHQQQQQQQQQQX-QQQQ-QQQQQQQQRQQQKQQKKQQ |
| 165 | K | 0.732 | # KKKRKKKKKKKTKKKKKKKKKKKKKKKKKKKEKEETKRRRKKKKKKRKKKTKKQKKKKKKRKKKRKKKRKKKRKKQQKQKKK |
| | | | KQKKKNQKKQQKKQQQKKKKKKKKKKKKKKKRKKKKKKRKKKKKKKKKKKKKKKKKKKKERKKKKKKKTKKT |
| 166 | E | 0.693 | # QKEEEEEQQEHEEVDKEEEEEEEEEAEEEEEQEEEETEEEEEEEAENEEEEDEEEEEEEEEEEEEEKEEEEEEEDEEEEAAE |
| | | | TETTEVEVTEEEAEEEEEEEDEEEEEEEEEEEQEEEKEEEEKEEEEKEEEEEEEEEEEADEEEEEEEEEEEQ |
| 167 | Y | 0.812 | # HYYYFYYYYYSYYYYYYSYYYYNYYYYEYYYYYYYYYYYYYYYYHYYYYQYYYYYYYYYHHYYYYYYYYHYYHYYYYYYHYY |
| | | | YYYYYYYYYYYYYYYYYYYYYYYYYYYYYYYYYYYYYNYYYYYYSHYYYYYYYYYYYYYYRHYYHYYYYYYH |
| 168 | A | 0.887 | # AAAAAAAAAAAAAAAAAAAAAAAAAAAAAAAAAAAAAAAAAAAAAAAAAAAAAAAAAAAAAAAAAAAAAAAAAAASSASAAA |
| | | | ASAAAASAASSAASSSAAAAAAAAASSSAAAAAASAAAAAAAAAAAAAAAAAAAAAAAAAVAAAAAAAAAAA |
| 169 | L | 0.832 | # ILLLLLLLLFVLLLLYHLLFLLLLLLLLLYLLLLLFLILLLTLLLLLLLLILLLLLLLLLLLLLLLLLLLLLLLLLLLLLTL |
| | | | LLLLLLFLLLLLFLLLSLLLLLLLLLLLLFLLLLLLLLLLLLLLLLLLLLLLLLLLLLLLLLLLLLLLLLLL |
| 170 | F | 0.946 | # FFFFFFFFFFFFFFFFFFFFFFLFFFFFFFFFFFFFFFFFFFFFFFFFFFFFFFFFLFFFFFFFFFFFFFFFFFFFFFFFFF |
| | | | FFFFFFFFFFFFFFFFFFFFFFFLFFFFFFFFFFFFFFFLFFFFFFFFFFFFFFFFLFFFFFFFFFLFFFFF |
| 171 | Y | 0.865 | # DYYYYYYYYYNYYYYYYYYYYYYYYYYYYYYYYHHYYYYYYYYYYNYYYYYYYNYYYYYYYYYYYYYYYYYYYYYNYYYYYY |
| | | | YYYYYYNYYNYYYYYYYYYYYYYYYYYYYYYYYYYYYRYYYYYYHYYYYYYYYYYYHYYDYYYYYNYYYYYY |
| 172 | K | 0.641 | # KDSKKKKSKKKKNKKNKKSKRKTKKKKKRSKKSEEKKKKKKKKKKRTKRAKKKRKKKKKKKKKKREAKKKKRKNTKKKKRSK |
| | | | RKRRKKKKRKKKRKKKRNKTRKKKTKKKAKKKKKKKKNKKRKKKKKKKKKKKKKKKKNKNKRKKRRKKKRKS |
| 173 | L | 0.873 | # LLLLLLLLLLLLLLVLLTLLLLALLLLLLLLLLLLLLLLLLLLLLLLLMLLVLLLLLLLHLLLLLLLLLLLHLLVLLLLLLL |
| | | | LLLLLLLLLLLLLLLLLLLSLLLFLLLLLLLLLLLLLLLLLLLLLLLLLLLLLLLLLLLLLLLLLLLLLHLL |
| 174 | D | 0.964 | # DDDDDDDDDVDDDDDDDDDDNDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDNDDDDDDDDDDDDDDDDDDDDDDDDDDDDDD |
| 175 | V | 0.793 | # VVVVVVVIVVVVVVVIVVVIVVVVVVVVVVIVVVVVVLLVVVVVVVIVVVLIVIVVVIVVVVVVVVLVIIIVVVVVVIVVVV |
| | | | VVVVIVVVVVVVVVVVLVVVVVIIIVVVIIIVVIVVVVVIIVVVVIVVVVVIVVVVVVVIIIVVVVILVVVV |
| 176 | V | 0.717 | # VEIVVVVVVVVMVVVVVVIIVVVVVVTIVIVLVVVVVVVVVVVVVVVIIVIVEVVVVVVVTVVVVVVVVTIVVVVIIIIVVI |
| | | | VIVVIIMVVIITVMIIVVVAVVVVVVVVVVVVVVVVVVEVVVVVVVVVVVVIVVVVVVVVVVVVVVEVMVVA |
| 177 | P | 0.808 | # PPPPQPPPPPSPPPPPPPPPPPPPQQPQPAQPAQQPPSPPPPPPPPPPPPPPPPPPPSPPPKPKPQSKPPPPPPPPPPPPPP |
| | | | PPPPPPPPPPPPPPPPQQPPPPPPPPPPSPPPPPPPPPPSPPPPPPPPPPPPPPPPPPPPPPPSPPPPPPPP |
| 178 | I | 0.885 | # IIIIIIVIIIIIIIIIIIIIIIIIIMIIIIIIIIIIIIIIIIIIIIIIIMMIIIIIIIIIIIIILVMLIIIIIIIIIIIIII |
| | | | IIIIIIIIIIIIIIIITIIIIIIIITIIIIIMIIIIIIIIIIIIIIIIIIIIIIIIIIIIIIIIIIIIIIII |
| 179 | D | 0.701 | # EDDDDDDDDDDEDDGNDDKDDGDDNDDGEDGDDDDDNDDDEDDDDDDDDDTDDNGNDGDNDEDEDDKDDDD-DDDDDDDDDD |
| | | | DDDDDDNDDDDDDNDDDDDDDGDDDDDDKDNDEDDKEDDDDNDDDDDNDDDDDGDNDDDDDDDDDEDDDDEN |
| 180 | N | 0.428 | # --N-GNKD-NNDD--DNND-NN-NNEN-D--K-K-DG--RGNKK-NKNNNSNDTKKKSN---N-N-DNK----DDNSDSDNN |
| | | | DSDDGDNYNSSDDDSSE-N-NNNNNNNNN-NNNNNGK-D-K---NNNNNNN-N-TN--NNN-NNKNNDHREK |
| 181 | N | 0.949 | # NNNNNNNNNNKNNNNNNNNNNNNNNNNNNNNNNNSNNNNNNNNNNNNNNNNNNNNNNNNNNNNNNNDNNNNNNNNNNNNNNN |
| | | | NNNNNNNNNNNNNNNNNNNNNNNNNNNNNNNNNNNNNNNNNNNNNNNNNNNN-NNNNNNNNNNNNNNNNNNN |
| 182 | T | 0.441 | # NSTNTNTDNNSTGADTTKTDTNSTTTTSTTKDTRTTTNDTTNATSKITTDPNSTTDTTNSSSTSTTINTDTTDATNKNSSSG |
| | | | SNSNDNDNSNNNSSNNSDTDTTTTDTTTNTNTTTANIATDTTTTNNTNINTANNNTDASTTTNTNNTATITK |
| 183 | S | 0.729 | # SSSSSTSTSTNSSTSTSTSTSTSSSSSTSSSTSSSSTTTSRSSRTTTSSTTTTSSTSSSTSSRSSSSSSTSTSTSSSSTSSS |
| | | | SSSSSSSSSSSDSSSSTSSTSSRSSSSSSSTSSSSSSSTSSTTSTTTTTTSSNSTSTSTSSSTSTTSTSTST |
| 184 | Y | 0.923 | # YYYYYYYYYYYYYYYYYYYYYYYYYYYYYYYFYYYYYYHYYYYFYYYYYYFYYYYFYYYYYYYYYYYYYYYYFFYYYYYYYY |
| | | | YYYYYYYYYYYYYYYYYYYYYYYYYYYYHYYYYYYYYYYYYYYYYYYYYYYYYYYYYYYYYYYYYYYYYYYY |
| 185 | R | 0.742 | # MRTKRRKRRRRTRRRRRMRKTRRRRRMRRRRRRTTRRRRRTRRMRRRRRRRMRMRITIRRRTRTRTRTTTARRRRRRRRRRR |
| | | | RRRRRRRRRRRRRTRRRRRRRRRRRRRRMRRRRTRRTRRRTRRTIRRRRRRRRRRRRRRRRTRRRRRRTRRR |
| 186 | L | 0.974 | # LILLLLLLLLLLLLLLLLLLLLLLLLLMLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 187 | I | 0.786 | # VKRIIVIIIIIIIIIIIIRTVIIIIIIIIRIIIKKIIIIIILIIIIIIIRIIIIIIITIIIIVIIKIIIIRIIILIIIIITI |
| | | | IIIIIIIIIIIIIVIIIIIIIIIIIIIITIIIIIIIIIIIIIIIIIIIIITIIIVIVIIIIIIIIIIIIIII |
| 188 | S | 0.657 | # SSSSNSSSSSSNNNNSSHSSNSGNNSNNSSSHSNNSSSSSNSSHSSNSNSRGSNSHHESSSSSSSNHSHNHSHHNNNNNNNN |
| | | | NSNNNNNNNNNNNHSNSSSSNNSSSSSSSSNSSNNNHHNSNSSNSSNNSSSSSSSSSSSNSNSSHSSSNSDS |
| 189 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 190 | N | 0.933 | # NNNNNNNNNNNNNDNNNNNNNNNNNNNNNNNNNNNNNNNNNNNNNNNNNNNNNNDNNNDNNNNNNNNNNNNNNNNNNNNNNN |
| | | | NNNNNNNNNNNNNDNNNNNNNNNNNNNNNNNNNNNNNNNNDNNNNNNNNDNNNNNNNNNNNNNNNNNNKNNN |
| 191 | T | 0.882 | # TTTTTTTTTTTTTTTTTRTTTTTTTTTTTTTTTTTTTTTTTTTTTTTTTTTTTTTTTATTTSTSTSTSSSTTTSTTTTTTTT |
| | | | TTTTTTTTTTTTTTTTATTTTTTTTTTTTTTTTTTTSRTTTTTTTTTTTTTTTTTTTTTTTTTTTTTTSTTT |
| 192 | S | 1 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 193 | V | 0.826 | # VVIVVVVVVVKVIVVVVVVVEVVVVVAVVVVTVVVVAVVVVVVVVVIVVVVVVVTVVVVVTVVVVITVVVVVVVVVVVVVVV |
| | | | VVVVVVVVVVVVVVVVVVVITVVVVVVVVVTVVVVVVVVVVVVVVVVVVVVVVVVVVVVVVVVVTVVVTTIV |
| 194 | I | 0.891 | # IIIIIIIIIIIIIIIIIIIIIIIIIIIIIIIIIIIIVIIIIIIIIIILIVIIIIVIIIIIIIIIIIIIIVIIIIIIIIIIII |
| | | | IIIIIIIIVIIIIIIIIVIIIIIIIIIISIIVIIIIIIIIIIIIIIIIIIIIIIIIIITIIIIIIIIIILII |
| 195 | T | 0.94 | # TTTTTTTTTTTTTRTTTTTTTTTTTTTTTTTTTTTTTTTTTTTTTTTTTTTTKKTTTTTTTTTTTTTTTTTTTTTTTTTTTT |
| | | | TTTTTTKTTTTTTTTTKTTTTTTTTTTTTTTTTTTTTTTTTTTTTTTTTTKTTTTTTTTTTTTTTTTTTTTT |
| 196 | Q | 1 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 197 | A | 0.987 | # AAAAAAAAAAAATAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 198 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 199 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 200 | K | 0.964 | # KKKKKKKKKKKKKKKKKKKKKKKKKKKKKKKKQKKKKKKKKQKKKKKKKKKKKKKKKKKKKKKEKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 201 | V | 0.816 | # VTVVVVVIVIIVVVVIVVVVVVVVVIVVVIVIVVVIVVVVVIVVVIVVVIVVIIVVVIVIIVVVIVVVAIVVIVVVVVVVVV |
| | | | VIVVVVVVVVVVVVIVVIIVVVVVVVVVVVVVVVVVVVVIVVVVVVVVVIVVVIVVVVIVVVVVVVIVVVVI |
| 202 | S | 0.861 | # SSSSSSSSSSSSTSSSSSSSSSSSTSSSTSTSTSSSSSSSSSSTSTSSTTSSSSSSSSSSTSTSSSSSSSSSSSSSSSTSSS |
| | | | SSSSSSSSSSSSSSSSSSSSSSTTTSSSSTSSTSSSSSSSSSSSSSSSSSTSTSSSSSSSSSSSSSSSSSTN |
| 203 | F | 0.974 | # FFFFFFFFFSFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFYFFFFFFFFFFFFFFFF |
| 204 | E | 0.92 | # EEEEEEEEEEEEEEEEEEEEEEEEEEEEEEEEEEEDEEEEEVEEEEEEEEEEEEEEEEEEEEEEEEEEEEEEEEEEEEDEEE |
| | | | EEEEEEEEEEEEEEEEEEEEEEEEEEEEEEEEEEEEEEEEEEEEQEEEEEEEEEEEEEEEEEEQEQEEEEEE |
| 205 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 206 | I | 0.987 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIITIIIIIIIIIIIIIIIIIIIIIIIIIIIIIIIIIIIIIIIIIIIIIIIIIIIIIIIIII |
| 207 | P | 0.987 | # PPPPPPPPPPPPPPPPPPPPPPPPPPPPPPPPPPPPPPPPPPPPPPPPPPPPPPPLPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 208 | I | 0.987 | # IIIIIIIIIIIIIIIIIIIIIIIIIIIIIIIMIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 209 | H | 0.987 | # HHHHHHYHHHHHHHHHHHHHHHHHHHHHHHHHHHHHHHHHHHHHHHHHHHHHHHHHHHHHHHHHHHHHHHHHHHHHHHHHHH |
| | | | HHHHHHHHHHHHHHHHHHHHHHHHHHHHHHHHHHHHHHHHHHHHHHHHHHHHHHHHHHHHHHHHHHHHHHHH |
| 210 | Y | 0.933 | # YYFYYYYYYYYYYFYYYYYYYYYYYYYFYYYYYYYYYYYYFYYYFYYYYYYYYYYYYFYYYYYYYYYYYYYYYYYYYYYFYY |
| | | | YYYYYYYYYYYYYYYYYYYYYYYYYYYYYYYYYYYYYYYYYYYYYYYYYYYYYYYYYYYFYYYYYYYYYYYY |
| 211 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 212 | A | 0.785 | # AAAAAAAAAAATTATATAAAAAAATAAAAAATATTTAAAAAATAAAPAAAATTATATATAATATATATTTAAAATAATATAA |
| | | | AAAAAAAAAAATAATATAAATTAAAAAAAATAATAATTTAATTAAAATAATAATAAAAAAATAATVAAAAAA |
| 213 | P | 0.987 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 214 | A | 0.974 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAATAAAA-AAAAAAAAAAAAAAAAAAAAAAAAAAAA |
| 215 | G | 0.987 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGWGGGGGGGGGGGGGGGGGGGGGGGGGGGG |
| 216 | F | 0.964 | # FYFFFFFFFFFFFFFFFFFFFYFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFLFFFFFFFFFFFFFFFFFFFFFFFFFF |
| 217 | A | 0.964 | # AAAAAAAAAAAAAAAAAAAAAAAAAAAAAAAAAAAAAAAAAAAAAAAAAAAAAAAAAAAATAAAAAAAAAAAATAAAAAAAA |
| | | | AAAAAAAAAAAAAAAAAAAAAAAAAAAAAAAGAAAAAAAAAAAAAAAAAAAAAAAAAAAAAAAAAAAAAAAA |
| 218 | I | 0.869 | # IIIIIIIIILIIIIIIMIIIIIIIIIIIIIIIIIILIIIIIILIIIIIILIIIIIIIILILIIIIIIIIIIIMIIIIIILLI |
| | | | LILLIIIIIIIIIIIIIIIIIIIIIIIIIIIIIIIIIILIILLIIIIIIIIIIIIIIIIIIIIIIMIIIIII |
| 219 | L | 0.946 | # LLLLLLLLLLLLLLILLLLLLLLLLLLLLLLLLLLLLLLLLLLLLLLLLLLLLILLLLILLILILLLLLLLLLLLLLLLLLL |
| | | | LLLLLLLLLLLLLLLLILLLLLLLLLLLLLLLLLLLLLLLLLLLLLLLLLLLLLLLLLLLLLLLLLLLLLLL |
| 220 | K | 0.889 | # KKKRKKKKKKKKKKKKKKKKKKKKKKKKKKKKKKKKKKKKKKKKKKKKKKKKQKKKKKKKKQKQKQKQQQQKKKKKKKKKKK |
| | | | KKKKKKRKKKKKKKKKKKKKKKKKKKKKKKKKKKKKKKKKKKKKKKKKKKKKKKKRKKKKKKKKKKKKKKKK |
| 221 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 222 | N | 0.767 | # RKNNNRKNKNRNNNNKKNNNKKNNNNKNRRNNKNNNNRNRNKNNNNNNNNKNKKNKNNNNKNKNNNNNNNNNNNNNNNNNNN |
| | | | NNNNNNNNNNNNNNNNNNKKKKNNNNNNNNNKKNKNNNNNNKKNKKKKNNNNKNNRNNKNNNNKNNNNNKNN |
| 223 | D | 0.769 | # DDDNNDDEDNEDDNNDDDDNDDNDDNDNDDDDDDDENDDDNDDDNDNNNNDDDDDDDNNDDDDDDDNDDDDNNNNDNDDNDN |
| | | | DNDDNNNDNDDDNDDDNDDDDDNDNNNNDNDDDDDDDNDDDNDNDDDDDDDDDNNDDDDDDNDDDNDDDDND |
| 224 | K | 0.894 | # KKKKKKEKKKEKKKKKKKKKKKEKKKKKNKKKKKKKKKKKKKKKKKKKKKKKKKKKKKKKKKKKGKKKKKKKKKKKKKKKKK |
| | | | KKRKKKKKKKKKKKKKKKRRKKKKKKKKKKKKKEKKKKKKKKKKKKKKKKKKKKKKKKKKKKKKKKKKKKKN |
| 225 | K | 0.669 | # KNKTTKKGKTDKNTKKRKKTEKTKKTKTKKMKQKKKTKKKTKKKTKTTTTKKGNKKKTTKRKKKTKKKKKKTTTKKTKKKKK |
| | | | KKKKKTTTKKKKTKKKTKNNKKKTTKKKKTKKTKKKKTKKKKKKKKKKKTKKKTTKRKKKKRKKKTKKKKTK |
| 226 | F | 1 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 227 | N | 0.881 | # NNNNNNNNNNNNNNNNNNNNNNDNNNNNNNNNNNNNNSSNNQNNNNDNNSNNNNNNNDNNNNNNNNNNNNNNSDNNNNNNNN |
| | | | NNNNNNNNNNNNNNNNNNNNNNNNSNNNNNNNNNNNNNNSNNNNNNNNNNNNNNNNNNNNNNNSNNSNNNNN |
| 228 | G | 0.974 | # GGGGGGGGGGGGGGGGGGGGGGGGGGGGGGGGGGGGRGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGEGGGGGGGGGGGGGGGGGGGGGGGGGGGGGG |
| 229 | T | 0.766 | # TTTTKTKKKKTTTKTTKKKTTTKTTTTKKTTTSTTTTKKTTKTKKTKTKKKTTTTTKKTTTTTTKTTTTTTKKKTTSTTTTT |
| | | | TTTTTTTTTTTTTTTTTTSTTTTKKTTTTTKTTTTTTKTKTTTTKTKTTKTTTTSTKTTTTTKKTSKTKTKT |
| 230 | G | 0.987 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGEGGGGGGGGG |
| 231 | P | 0.731 | # PNPPPPLPLPLPPPPPQPPPPPPPQPPPQPPPPPPPPTTPPPPTPPRPSPEPTPTPPPPPPPLPQPPPPPPPPLPPPPPPPP |
| | | | PPPPPPPPPPPPPPPPTPPLPSPSPPPPPPPQPPPPPPPSPPPPTPLPPPPPPPPPPPSPPPTPQPSPPPPP |
| 232 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 233 | T | 0.602 | # IKKTAKTKKTKKKTTKKKTTKTTTKTKTNTKTTTTKTNNKTNTNKTPTNTTRTKTKTTKTESKSRSTSTKSTTTTKTKTTTT |
| | | | TKTTTTATKTTTTTKTTTTKKKTKTKKKNTKKTTKTKTTKTTTTKTTTTKTKKTTTTTKKETTIKTKKKTTK |
| 234 | N | 0.936 | # NNNNNGNNNYNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNINNNNNNNNNNNNNNNNNSNNNNKNNNNNNNNNNNNNNNNNNN |
| 235 | V | 0.987 | # VVVVIVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 236 | S | 0.987 | # STSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 237 | T | 0.955 | # TTTTTTTTTTTTTTTTTTTTTTTTTTTTTTTTTTTTTTATTTTTTTTTTTTTTTTITTTTATTTTTTTTTTTTTTTTTTTTT |
| | | | TTTTTTTTTITTTTTTTTTTTTTTTTTTTTTTTTTTTTTTTTTTTTTTTTTTTTTTTTTTTTTTTTTTTTTT |
| 238 | V | 0.974 | # VVVIVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVAVVVVVVVVVVVVVVVVVVVV |
| 239 | Q | 0.964 | # QHQQQQQQQQQQQQQQQQQQQQQQQQQQQQQQQQQQQQQQQLQQQQLQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 240 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 241 | T | 0.974 | # TTTTTTTTTTTTTTTTTTTTTTTTTTTTTTTTTTTTTTTTTTTTTTTTTTTTTTTTTTTTTTTITTTTTTTTTTTTTTTTTT |
| | | | TTTTTTTTTTTTTTTTTTTTTTTTTTTTTTTTTATTTTTTTTTTTTTTTTTTTTTTTTTTTTTTTTTTTTTT |
| 242 | H | 1 | # HHHHHHHHHHHHHHHHHHHHHHHHHHHHHHHHHHHHHHHHHHHHHHHHHHHHHHHHHHHHHHHHHHHHHHHHHHHHHHHHHH |
| | | | HHHHHHHHHHHHHHHHHHHHHHHHHHHHHHHHHHHHHHHHHHHHHHHHHHHHHHHHHHHHHHHHHHHHHHHH |
| 243 | G | 0.987 | # GGGRGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 244 | I | 0.987 | # IIIIIIIIIVIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 245 | R | 0.858 | # RRRRRRRRRKRKRKRRRRRRRRRRRRRRRKRKRKKRRRRRRRRRRRRRRKRRRRRRRRRKRRRRRRKRRKRRRRRKRRKKRR |
| | | | RKRRRRKRRRRRRRKRRKRRRRRRRRRRRRRRRKRRRRRRKKRRRRRRRRRRRRRKRRKRRRRRRRRRRRRK |
| 246 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 247 | V | 0.987 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVIVVVVVVVVVVVVVVVVVVVVVVVVVVVVVVVVVV |
| 248 | V | 0.987 | # VVVVVVVVVVVVVVVVVVVVVVVVVVVVVVVVIVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 249 | S | 1 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 250 | T | 1 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 251 | Q | 0.987 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQHQQQQQQQQQQQQQQQQQQQQQQQQQQQQ |
| 252 | L | 0.977 | # LLLLLLLLLLLLLLLLLLLLLLLLLLLLLLLLLLLLLLLPLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLPLLLLLLLLLLLLLLL |
| 253 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 254 | L | 0.987 | # LLLLLLLLLLLSLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 255 | N | 0.961 | # NNNNNNNNNYNNNNNNNNNNNNNNNNNNNNNNNNNNNNNNNNNNNNNNNSNNNNNNNNNNNNNNNNNNNNNNNNNNNNNNNN |
| | | | NNNNNDNNNNNNNNNNNNNNNNNNNNNNNNNNNNNNNNNNNNNNNNNNNNNNNNNNNNNNNNNNNNNNNNNN |
| 256 | G | 0.974 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGKGGGGGGGGGGGGGGGGGGGGGGGGGGGGGA |
| 257 | S | 0.987 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSNSSSSSSSSSSSSSSSSSSSSSSSSSSSSSSSSSSSSSSSSSSSSSSSSSSSSSSSSSSSSSSSSS |
| 258 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLILLLLLLLLLLLLLLLLLLLLLLLLLLLLLLLLLLLLLLLLLLLLLLLLLLLLLLL |
| 259 | A | 0.968 | # AAAAAAAAAAASAAAAAAAAAAAAAAAAAAAAAAAAAAAAAAAAAAAAAAAASSAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 260 | E | 0.977 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEGEEEEEEEEEEEEEEEEGEEEEEEEEEEEEEEEEEEEEEEEEE |
| 261 | E | 0.805 | # EEEEKEGEEEKGEEEEEKEEGETEEEEEEEKEEEEEEEEEEEEEDEEEEGEEGGEEKEEEEEEEEGEEKEEEEEEEEEEEEE |
| | | | EEEEEEEEEEEEEEEEEEDKEEEEKEEEEEEEEKEEEEEEGKEEEEEEKEEEEEEEKEEEEEEEEEEEEEKE |
| 262 | E | 0.844 | # EDEEEEEEEEEEEEEGEEEEEEEEEEEEEEEEEEEEDEEEEEEEEEEEEEEEEDEEEKGEEDEDEEEDEEEEDEEEEEEEEE |
| | | | EEEEDEGEEEEEEEEEEGDEEEEEEEEEEDEEEEEEEEEEEKEEEEEEEEEEEEEEEEEEEEEEEDEEDEED |
| 263 | V | 0.835 | # IIVVIVVVVVVVIVVVVVVVVVVVVEVIIVVVVVVVVVVVVVIVVVVVIVVVVVVVVVVVVIVIIVIIIIIIVVVVVVVVVV |
| | | | VVVVIVVVVVVVVVVVIVVIVVVIVVVVVVVVVVIIVVVVVVVVVVVVVVVVVVIVVIVVVVVVVIVVIVVV |
| 264 | V | 0.901 | # VVVVVVIVVVAVVVVVVVVVVVVIVVVVVVVIVVVVVVVVVVVVVVVVVIVVVVVVVVVVVVVVVVVVVVVVVVVVVVIVVV |
| | | | VVVVVVVVVVVVVVIVLVVVVVVVIVVVVVVVVVVVVVVVVVVVVVVVVVVVVVVVVVVVVVIVVVVVVVIV |
| 265 | I | 0.908 | # IIIIIIIIIIIIIIIIIIIIIIIIIIIIIIIIIIILIIIIIIILIIILILIILIIIIIIIIIIIIIIIIILIIIIIIIIIII |
| | | | IIIIIIIIIIIIIIIIIIIIIIIIIIIILIIVIILIIIIIIIILIIIIIIIIIIIIIIIIIIIIIIIIIILI |
| 266 | R | 0.946 | # RKRRRRRKRRRRKRKRRRRRRRRRRRRRRRRRRRRRRRRRRRRRRRRRRRRRRRRRRRRRRRRRRRRRRRRRRRRRRRRRRR |
| | | | RRRRRRRRRRRRRRRRKRRRRRRRRRRRRRRRRRRRRRRRRKRRRRRRRRRRRRRRRRRRRRRRRRRRRRRR |
| 267 | S | 0.974 | # SSSSSSSSSSSSSSFSSSSSSSSSSSSSSSSSSSSSSSSSSSSSSSSSSSSSSSSSSSSSSPSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 268 | E | 0.597 | # EAAADEEDDDAEEESEDDDVETDSEEEDDKASEEEAEDDVEDEEEADEEDENAEEEEDEEKRSEEEEEEEEEDDSEEEEEEE |
| | | | EEEEEEEEEEEEEEEEEVEESEEDDAAAAADAVEEEEDEEEKEEDEDEEDEEEEEDDEAEEEDDAEEEESEE |
| 269 | N | 0.977 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNDNNNNNNNNNNNNNNNNNNNNNNNNNNNNNNNNNNDNNNNNNNNNNNN |
| 270 | F | 0.887 | # FLFFFFIFFFFFFFFFFFFFFFFFFFLIFFFFFFFFIIIFFFFFFFFFFFFFFFFFFFFFFFFFLFLFFFFFFFFFFFFFFF |
| | | | FFFFFFFFFFFFFFFFIFFFFFFFFFFFFFFFFFFFFFFFFFSFFFFFFFFFFFIFFFFFFFFFFFFFFFFF |
| 271 | T | 0.822 | # TSSSTTTTTTMTTSTTTTSTTTSTSTTTTSSTTTTSSTTSTSSTTTSTMTTSSTTTTTTTSTTTTTTTTTTTTTTTTTTTTT |
| | | | TTTTTTSTTTTTTSTTTTTTTTTSSSSSSTTTTTTTTTTTTTTTTTTTTTTTTTTTTTSTTTTTTTTTTTTS |
| 272 | D | 0.761 | # DDDDDNNNNDDDDDDDDDDDNDNDNDDDDDDNNNNNDNNDNDNDDDDNDNNDDNNNNDDDNNDNDNDN-NNNDDDNNNNDDN |
| | | | NNNNNNDNDNNNNDNNNDNDDNNNDDDDDDNNNDDDDNNDNDDDDNNDNNNDNNNDDNDNDNDDNDNNDDNN |
| 273 | N | 0.977 | # NNNNNNNNNNSNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNSNNNNNNNNNNNNNNNNN |
| 274 | A | 0.776 | # ATTAAAAAAANAAAAAATAAATAAATATATTAAAAAAAAAAAAVAAAAAAAAAIAIAVVAAATAAAAAAAAAATAAAAAAAA |
| | | | AVAAAAAAAVAAAAAAATAAAAVATTTTAAAAAAAAVAAAVAAAAAAAAAAAAAAAAAAAIAVAAAAAAAAV |
| 275 | K | 0.943 | # KKKKKKKKKKKKKKRKKKKKKKKKKKKKKKKKKKKKKKKKKNKKKKKKKKKKKKKKKKKKDKKKKKKKKKKKKKRKKKKKKK |
| | | | KKKKKKKKKKKKKKKKKKKKKKKKKKKKKKKKRKKKKKKKKKKKKKKKKKKKKKKKKKKKKKKKKKKKKKKK |
| 276 | T | 0.743 | # TTTVSTTTTTNTTTVTTTVTTNTTTTTTTITITITTTTTTTTTTTITTITTTTNNNTTTTTTTTTTNTTTTITTVSTTTTTI |
| | | | TSTTTTTTTTTTITTNTTTTNTTTTTTTTTTITTTTTTTTTTTTTITITTTTITTITTTTNTTTTTTVNITI |
| 277 | I | 0.987 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIILIIIIIIIIIIIIIIIIIIIIIIIIIIIIIIIIIIIIIIIIIIIIIIIIIIIIIIIIIIIIIIIII |
| 278 | I | 0.987 | # IIIIIIIIIIIIIIIIIIIIIIIIIIIIIIIIIIIIIIIIIIIIIIIIIIIIIIIIIIIIIIIIIIIIIIIIIIIIILIIII |
| | | | IIIIIIIIIIIIIIIIIIIIIIIIIIIIIIIIIIIIIIIIIIIIIIIIIIIIIIIIIIIIIIIIIIIIIIII |
| 279 | V | 0.977 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVIVVVVVVVVVVVVVVVVVVVVVVVVVVVVVVVVVVVVVVVVVVIVVVVVVVVVVVV |
| 280 | Q | 0.921 | # QQQQQQQQQQQQQQQQQHQQQQQQQQQQQQQQQQQQHHQQQQQHQQQQQQQQQQQQQQQQQQQQQQQQQQQQQQQQQQQQQH |
| | | | QQQQQHQQQQQQQQQQQQQQQQQQQQQQQQQQQQQHQQQHQQQQQQQQQQQQQQQQQQQQQQQQQQQQQQQH |
| 281 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 282 | N | 0.722 | # NNNKNNKNKRNNNNNNKNNNNNTNNKNNNNNNNNKNKNNKKNTNNNKKNSNNKKNNKKNKKNNNKNNNNNNNNNNNNNNNNN |
| | | | NNNNNKSNNNNNNNNNNKKPKNNKNKKKNNNNRNNNNNNNNNNKNNKNDKKNKNKKNNKNNKNKKNRNNNNN |
| 283 | E | 0.598 | # QEEEEEDTEDEKKKEEEEETEEEEEEEKKEEKEENEEEEETKKEEEEEETQEEEEEEEEEDAEAEAKAATAVEEEEDEEDNK |
| | | | DKDDTKKKDEEKEEEEKEEEEEEEEEEEHTEEEEEEAEVEEKEEEEEEEEEEEEEEEEEEADEEEEEKVEEK |
| 284 | S | 0.647 | # TSSPSAPSSPSSSSSTSSSSTSPSPATSSTTSTSPSASSAASPTSSSATSSSPSTSPATPPSSSPSSSSSSSSSSSSSSSSS |
| | | | SSSSSSSTSSSSSSSSSPAISTSPSPPPSSSSASSSSSSSSSSASSASSSSSSTPASSATSPSPSSPPSTTP |
| 285 | V | 0.908 | # VIVVVIVVVVVVIVVVVVVVVIVVVVVVVVVVVVVVVVVVVVVVVVVVVVLVVVVVVVVVVVVVVVIVVVVVVVVVVVVVVV |
| | | | VVVIVVVVVVVVVVVVVVVVVVVVVVVVVVVVVVVVVVVVVVVVVVVVVVIIVVVVVVVVVVVVVVVVIVIV |
| 286 | E | 0.545 | # KAEEEAEEKQKVSEEEEEEEKEVVEEEEEEQEEEKVEEEEVEVEEVQEEMEVQEEEEEKVVEQESEEEEEEDVAEVVVVVVV |
| | | | VVVVVEVVIVVVVAVKKEQHEEEKAEEEEEVQEEEEQAEQEKEEEEEEVEEAVEEEEVEEEKEEEVKEEEKA |
| 287 | I | 0.987 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIISIIIIIIIIIIIIIIIIIIIIIIII |
| 288 | N | 0.872 | # NNNNHNNTNNSNNNKNNNNNHNNNNNNNNNHNNNNNNNNKNNNNNNNNNKNNNTNNNNNNNNNNNNNNNNNNNNNNNNNNNN |
| | | | NNNNNNNNNNNNNNNNNNNNNNNNDNNNTNNNNNNNNNNNNNNNNNNNNNNNNNNNNNNNNNTNNNNNNNNN |
| 289 | C | 0.987 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 290 | T | 0.853 | # TTTTMTTVTTITTTITTTTTITITTTTTTTTTTTTTTTTTTITTTTTTTSTTTMTTTTITTTTTTTTTTTTTTTTTTTTTTT |
| | | | TTTTTTTTTTTTTITTTTTTTTTTITTTTTTITTITTTTTTTTTTTTTTTTTTTTTITTTTTITTTSTTTTT |
| 291 | R | 0.987 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRKRRRRRRRRRRRRRRRRRRRRRRRRRRRRRR |
| 292 | P | 0.96 | # PPPPPPPPPPPPPPLPPPPPPPPPPPPPPPPPLPPPPPPPPPPPPPPPPPPPLPPPPPPPPPPPPPLPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 293 | N | 0.794 | # NGNNNSNNNANNNNNNNNNNNNNNTNNNNNNNNNHNNDNSNNNGNNNSNSNNSNNGNNNSSNNNNNNNGNNNNNNNNNNNNN |
| | | | NNNNNGNNNNNNNNNNNNNNNNNGSNNNNYNNSNNYNNNNNNNNNNNNNNNNNNNNNNNNNNNNGNNNNNNN |
| 294 | N | 0.929 | # NNNNNNNNNNNNNNNNNNNNNNNNNNNNNNNNNNNNNNNNNNNNNNNNNNNNNNNNNNNKDNNNNNNNNNNNNNNNNNNNNN |
| | | | NNNNNNNNNNNNNNNNNNNNNNNNNNNNYNNNNNNNNNNYNNNNINNNNNNNNNNNNNKNNNNNKNNNNNNN |
| 295 | N | 0.829 | # NRNNNSNNNNNSNNNNYNINNNNNNNNNNNNNNNNNNNNNNNNNKNNNNNNHNNNNNNKTKNNNNNNNNNNNNNNNYNNNNN |
| | | | NNNNNNNNNNNNNNNNINNNNNNNNNNNNNNNNNNNNNHNNNNNYNNNNNNNNNNNNNKNNNNNKNNNNNNN |
| 296 | T | 0.886 | # TTTTTTTTTTTKTTTTTTTTTTTTTTTTTTTTTTTTTTTTTTTTTTTTTTTTTTTTTTTTTTTTTTTTTTTITTTTTTTTTT |
| | | | TTTTTTTTTTTTTTTTSTTITTTTTTTTEKTTTTTVTTTKTTTTRTTTTTTTTTTTTTTTTTTTITTTTTTT |
| 297 | R | 0.888 | # RRRRRGRRRRRRRRRRSRRRRRRRRRRRRRRRRRRRRRRRRRRRKRRRRSRRRRRRRRRRRRRRRRRRRRRRRRRIRRRRRR |
| | | | RRRRRRRRRRRRRRRRRGRRRRRRRRRRTRRRRRRRRRRRRRRTKRRRRRRRGRRRRRRRRRRRRRRRRRRR |
| 298 | K | 0.775 | # QRKKKQKRKEKKKKKRKKRKKKRKKKKKRKKNKKKKKKKKKKKKRKKKKKKKKKKRKKKRKKKKKKQKRKKKKKKKKKKKKR |
| | | | RKRKKKKEKKRKRKKKRKRKKKKKKKKKKKKKKKKRKKKKKKKRGKKKKKKKKRKKKKKRKKNKRRKKKKKK |
| 299 | S | 0.679 | # GRSSGSSSSSSTSGSSRGSRSSSSSGSSGGSRGSSSSSSSGGSRSRSSSSRSSGSSSGRRRSSSSSSSSGSRGGSSHSSGSS |
| | | | DSDGSSSSGSSSGSSSSGSGSSRSSSSSRSSSSSSSSSRRGSSSRGSSSSSSSSSSSSRSGSSSRRSSKGGS |
| 300 | I | 0.886 | # IIIIIIIIIIMLIIIIIIIIIIIIIIIIIIIIVIIIIIIIIIIIFIIIIIIIIIIIIIVIIIIIIIIIIIIIIIIIIIIIII |
| | | | IIIIIIIIIIIIVIIIMIILIIIIIIIIIIIIIIILIIVIIIIIIIIIIIIIIIIIIIIIIIIIILIIIIII |
| 301 | H | 0.45 | # PTHSYRHPTHTHNHHHRHHRHHHNHHHSQHHSQHHSHHHPRPPAHSHPNHSHRNPTAYTHHRTRNHSRSHRHHHPRHHHTPH |
| | | | THAHHHHNTHHPTHHHHHNRHHTSHHHHRRNHHPHSTYTHAHHHHHHPHHHHNNHNNNTNHTPPHSPPTTHH |
| 302 | I | 0.702 | # ILILVIIIIILMIILIIIVIIMIIIIIIIIIILIIFMIIIIMIIIMIIIMILIIMIIMMIIIFIIIIIIMIIIILMLIIIIM |
| | | | IIIIIILIILMIIIMLIIIIIIMIIMMMIIIIIIIIKILIIIIIIIIIIIIIIMIIIITIMIIIIIMILLIM |
| 303 | G | 0.93 | # GGGGGGGGGGGGGGGGGGGGAGGGAGGGGGGGGGGGGGGGGGGGGGGGGGGGGGGGGGGGGGGGGGGGGGGGGGGGGGGGGG |
| | | | GEGGGGGGGGGGGGGGGGGPGGGGGGGGGGGGGGGGGGGGGGGGGGQGGGGAGGGGGGGGGGGGGGGGGGGG |
| 304 | P | 0.876 | # PPPPPQPPPPPPPPPPAPPPPPPPPPPPPPPPPPPPPPPPPPPPPPPPPPPPPPPPPPPPPPPPPPPPPPPPPPPIAPAPPP |
| | | | PAPPPPAPPAPPPPPATPPGPPPPPPPPYPPPPPPPPPPPPPPPPPPPPPPPPPPPPPPPPPPPPPPPPPPP |
| 305 | G | 0.927 | # LGGGGRGGGGGKGGGGRGGGGGGGGGGGGGGGGGGGGGGGGGGGGGGGGGGGGGGGGGGGGGGGGGGGGGGGGGGRRGGGGG |
| | | | GGGGGGGGGGGGGGGGGGGRGGGGGGGGGGGGGGGGGGGGGGGGGGGGGGGGGGGGGGGGGGGGGGGGGGGG |
| 306 | R | 0.692 | # GRRRRRRRKRKRRRKRRRRRRKSRRSQRRRRRSRRSRRRRRRRRRRERRRRRSRKRRRRRRSRSRSRSRRSRRRQRKRKSSK |
| | | | RKKKRRRRRKKRRRRRRRRARRRRRKKKRRRRRRRRRRRRRQQRRGRRRRRRGKRKRRRRRKKRRRRRRRRK |
| 307 | A | 0.734 | # SVAAHAAAAAVAAAAAAAAAAAAAAAAAAAAAAAAAAAAAAAAAAVAAAASWAAAATRVASAAAATAAATTAAAAAAAAVVV |
| | | | VAVVAAAAVAAAVAAAVAAWATVAAAAASAAAAAAAVAVATAAAAAAAAAAAATAAAAVAAAAAAAAAVVAA |
| 308 | F | 0.631 | # FYFFIFFFFFFFLIWIFVIFFFFFFFFFFFFFWFFNFFFFFFFVFYLWFFFSFWFFVFYFFFFFFILFFFFFVFWFFFLFFF |
| | | | FLFFFFLFSLLFFFLLFFLWFFYFFFFFFFIIFFFFIFWFLLLFFFFFFFFVFLFLFLYFFFIFFFFFLFFF |
| 309 | Y | 0.813 | # YYYYYYYYYYYYYFYYYYYVYYYYYYYYYYYHYYYFYYYLYYYLYFYYYVVYYYHYYYYVYYYYYYYYYYYYYYYYYYYYYY |
| | | | YYYCYYYYYYFYYYYYYYYYYYYYYYYYVVYYYYYRYHYYYYYYHYYYYYYYYYYYYYYYYYYYYYYYYYYY |
| 310 | T | 0.748 | # ATTAAAATAATAATTATTTTATTTTTAAAAATAAAAAATTVTAATTTAAATLATAAATTTTATAAATAAAATTATTTTTTTT |
| | | | TTTTAATYTTTATTTTTATATTTTATTTTTTATAATATTTAAAATTTTTTTTTTTTTTTATTTTTATATTTT |
| 311 | T | 0.741 | # TTTTTTTTTTTTTTTTKTTITTTTTTTTTTTTTTTTTTTTTTTT-TTTTTTATTTTTTTTTNTNTTTNTTTTTAT------- |
| | | | ----TT-T---T----XTTTTTTTTTTTVITTTTT-TTTTRTTTTTTTTTTTTTTTTTTTTTTT-RTTTTTT |
| 312 | G | 0.734 | # TGGGEGG-X-GDGGGGGGGGGGGGGGGGGGGKGGGGGGGGDGGR-G-GGEGKGGGGDGGKKGGGGGGGGGGGGRGRGGGGGG |
| | | | GGGGGGGGGGGGGGGG-GGRGGGGGGGGRGGGGGGRGGGKEEGGR-GGGGGGG-GGGGGGGGGG-RGGGGGG |
| 313 | E | 0.374 | # EEQSKKDEED-IEEQDKR-KNEQDAEEDDEQQGEEAEDGERQDQEEDEDRQDEEDRRREQTDSDDDQDSDAQRKQEEEEEEE |
| | | | EEEEEDEEEEEEEDEEQNAGEDQET---KKAGNDDEQRENKAADQKDEEEEEKDEEEEE-DEKEDNQEEEED |
| 314 | I | 0.845 | # IIVIIIIIIIIGIIIIIII-IIIIIIIIIIIIIIIIIIIIIINIIIIIIVIIVIIIIIIIIIIIIIIIIIIIIIIIIIIIII |
| | | | IIIIIIIIIIIIVIIITIIIIIVIVVV--IIIIIIIIIIIIIIIIIIIIIIIIIIIIIIIIIIIRIIIIIVI |
| 315 | I | 0.831 | # IMIIVIIIIITGIIIIIVIIIIIIIIIIIIIIIIIIIIIIIIIIVIIIIIIIIIIIIIIARIIIIIIIIIIIIIIIVIIIII |
| | | | IIIVIIIIVIIITIIIIIIIIIIIIIIILIIIIIIIIILIIIIIIIIIIIIIIIIIIIVIIIIIVIIIIVII |
| 316 | G | 0.964 | # GGGGGGGGGGGYGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 317 | D | 0.789 | # NDDDDDNDDDDTNDDDDDKNDDDDDDDNDDDDDDDDDNNDDDDDDDNDDNDDDDDDDDDDDDDDDDDDNDDNDDDDDDDDDD |
| | | | DDNDNDDDDDDNDDDDNDDKDDDNDDDDDNDDDDDIDDNTDNDDNDDEDDDNDDDDDDDDDDDNDDDDDDNN |
| 318 | I | 0.93 | # IIIIIIIIIIIGIIIIIIIMIIIIIIIIIIIIIIIIIIIIIIIIIIIIIIIIIIIIIIILIIIIIIIIIIIIIIIIIIIIII |
| | | | IIIIIIIIIIIIIIIIIIIMIIIIIIIIRMIIIIIIIIIIIIIIIIIIIIIIIIIIIIIIIIIIIIIIIIII |
| 319 | R | 0.977 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRKRRRRRRRRRRRRRRRRRRRRRKRRRRRRRRRRRRRRRRRRRRR |
| 320 | Q | 0.829 | # KKQLQHQQQQKQQQQQQLQQQQQQQQQQQQQQQQQQQQQQQQQQQKQKQQQKQQQRQQQQQQQQQQQQQQQQQQQQQQQQQQ |
| | | | QQQQQQQQQQQQQQQQQQKQQQRQQQQQQQQRQQQQKKQQQQQQQQQQQQQQQQQQQQQEQQQQQQQKRKQQ |
| 321 | A | 0.974 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAVAAAAAAAAAATAAAAAAAAAAAAAAAAAAAAAAAAAAAAAAAAAAAAAAAAAAAAAAAAAAAA |
| 322 | H | 0.878 | # YHHHHHYYHHHHHYHHHHHHHHHHHHHYHHHHHHHHHHHHHHHHYHHHHHHHHHHHHHHHQHYHHHYHHHHHHHHHHHHHHH |
| | | | HHHHHHHHHHHHHHHHHHYHHYHHHHHHHHHYHHHHHHHHHHHHHHHHHHHHYHHHHHHHHHYHYHHYHHHY |
| 323 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 324 | N | 0.86 | # NNNNNNTNNNNNNTNNNNNNNNNNSNNNDNNNNNNNNNNNNNNNNNNTNNNNNNNNNNNNNNNNNNNNNNNNNNNNNNNNNN |
| | | | DNNNNNNNNNNNNNNNNNNNNINTTNNNTHTNNNYNNNNNNNNNNNNNTNNNNNNNNNNNNNNNNNNNNNNN |
| 325 | I | 0.692 | # VLIIIILILSIIILLIIIIILLILILVIVILLIILLIVVIIILIIILIILLVLIILLIIILIIILILIIIIILLLLLLLLLL |
| | | | LLLLLLLLLLLLVVLLLIIIILLIIIIIMILIIVIILIIILIIIIILLLILIILIILLLILLIIIIIILIII |
| 326 | S | 0.884 | # SSSSSSNTSSSSSNSSSSSSSSNSSSSSSSTSSSSSSSSSSSSSSSSNSTSSSSSSSSSNNSSSNSSSSSSSSSSSSSSSSS |
| | | | SSSSSSSSSSSSSSSSSSSSSSSSNSSSNSNSSSSSSSSSSSSSSSSSNSSSSSSSSSSSSSSSSSSSSSSS |
| 327 | R | 0.56 | # KKRRRGRKRRRRARSRGRRRLEGRKRRGGQRRRGRITKEGGRKRRRRRRRGEKKRRSKRRKKRAKGKKKKRKRRSRRRRSNR |
| | | | RRSSRSRRSRRRRRRRKIRRRRRRRRRRRRKRRKKRRRRRRKRRRIKRRRRRRRRREKRGRRRRGRKGRKSR |
| 328 | A | 0.542 | # KAEATAAAVAKATATAAAAAAATAGAAAAAAAVVETTATEKATQAETAAASGVGTATEAATAARTAVAEAETAATTTTAATA |
| | | | TATTAVATATAATATTATAVTASATTTTTATTGAAAAAAAAAAAAAATTAATAAAAATATEIVATATSTVTQ |
| 329 | K | 0.485 | # DDKKNKRNDEEKEQKTKRKKEKQDKKKTTKNTKKDEKKKKDQDKKDQKKDQQQNKQKKERLKKKDKSKKKKRKQKQQKQKDA |
| | | | KQKRDQDQKQQDRKHQEKKKKKKNQKKKKKQKKKNKQQQKKKKKKKNKEKKKQKKKKQKKKEKKKKNKSKDE |
| 330 | W | 0.977 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWW$WWWWWWWWWWWWWWWWWWWWWWWWWWWWWW |
| 331 | N | 0.656 | # NNNNTDNTNINNNDNNDNNNNNNNNNTNENTENNNNNGKNNNNNNNNNNNNNNTNNNNNNSENENENEEENNENRNNNDNNN |
| | | | DNNNNNKNNNNDKNNNNNNNNEENNNNNDNDNNNTNNNNNNNNNENNDNNNTNNNNNENNENNNKNTNNHGN |
| 332 | N | 0.597 | # DNNESNDDEKKDNDNNSKNNNNKNNDNNENNKNKNNKNKNENNKNKNNNRNKDKKNNNKAANNNNNNNNNDNNNNNNDNNNN |
| | | | NNNNNDENSNNNNDNNNNENNDNSQNNNNANNNSNNNNNDDNNNDNNNNDDSNDDNHNDNNKNNNNNNNNKE |
| 333 | T | 0.94 | # TTTTVTTTTTTTTTTTTTTTTTTTTTTTTTTTTTTTTTTTATTTTTTTTTTTTTTTTTTTTTTTTTTTTTTTTTTTTTTTTT |
| | | | TTTTTTATTTTTTTTTTTTTTTTTTTTTATTTTTTTTTTTTTTTTTTATTTTTTTTTTTTTTTTTTATTTTT |
| 334 | L | 0.987 | # LVLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 335 | K | 0.434 | # SRNQRQKQRQEKEKKKRNKKYGSKQNKRKKKEENKTGRKGTKKKHKKKGEKRERKKKHEKKRRRGRKRRRKNKKEKKKKKKE |
| | | | QKKKTKNXKKKTKEKKPKKENQKQDKKKKKRKKEEKKEQRKKKKKKKQAKKEKKRKEERKKEKQEQKKKKEL |
| 336 | Q | 0.772 | # KKLQQQQKQQRQQMQKQQQQQQLQRQQLQQQQKQQKQKQQQQQHQQQQKKRQQQQQQQQQQQQQQQQQQQQQQQQQQQQQQQ |
| | | | QQQQHQQQQQQHQRQQQQQQQKQQRQQQQQQQQQQQQQQQQQQQQQQQKQQQQQQQQQQQQQQQKQQQQRKR |
| 337 | I | 0.788 | # VIIIIIIVIVIIIIIIIIIIIIIVIIIIIIIIIIIIVVVITIIIVIIIIVIIIIIIIIIIVLIIVLIIILLIIIIIIIVIVI |
| | | | IIVIIITVVIIIVIIIIIVIIIIIVVVVIIIIIIIIVIIIIIIIIIIIIIIIIIIIIIIIIIIIIIVIIVVV |
| 338 | V | 0.718 | # AAVVAVAAAVAVVVTVVFVAVVVVVVVAAAVAVVVVVIGVVVVVVVAVVVVVVVVAVVAVAAAAVAAAAAVAVVTVVVVVVV |
| | | | VVVVVVVVVVVVEIVVVVVVAVVAIVVVAAATVVVVVVAVVVVVVVVATVVVVVAVAAVAVVVVVVVVVVVV |
| 339 | I | 0.352 | # KISKVNEIITIKTEKTKRTSTADKIITKRIETTEEKKKEKAIEANKIGIKEEKAITTINGIIVIKIAIIIIKTIEITIRIII |
| | | | IIIMRIKRTIISIIITKIDKTTTIREEESSIKSKEETKTSTKKIEKIKEIIKEIKTKIIQEKGIAIEKEEEE |
| 340 | L | 0.926 | # KKKKKFKKKKKKKKKKKKKKKKKKKKKKKKKKKKKKKKKKKKKKKKKKKKKKKKKKKEKKKKKKKKKKKKKKKKKKKKKKKK |
| | | | KKKKIKKKKKKKKKKKKKKKKKKKKKKKKKKKKKKKKKTKKKKKKKKKKKKKKKQKKKKKKKKEKKKKKKKK |
| 341 | L | 0.974 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLFLLLLLLLLLLLLLLLLLLLLLLLLLLLLLLLLLLLLLLLLLLLLLLLILLLLLLL |
| 342 | R | 0.692 | # GRRERKGRGKGKGRRGRRRRRKKRKRRRRKRRRKKRRGRKRRRKRRSGRGGKGRRRKRRRRRRRRRRRGRGRRRKRRRRRRR |
| | | | RRRRKGRRRRRRKRRRRRGGKGRRKRRRRRRQRGRRRRRKKKKRRRRGRRRRRRRRRKRRGKRKRRGRRRRG |
| 343 | E | 0.823 | # EEEEEEEEEKEEEEEEEEEEDEKEKEEEEEEKEEEDEEEEEEEEEEEEEEEEEEEEEKKKEEEEEEEEEEEEEEEEEEEEEE |
| | | | EEEEEEEEEEEEEKEEEEKDEEVKKEEEEEEEEEEEEEEEEVVDEEQKEEEEEEEEEEEEEEEEEEEEEEKE |
| 344 | F | 0.795 | # QNQQRQQQQQQQQQQQRIQQQQQQQQQQQQQQQQQQHHHQQQEQQQQQQQQQQQQQQQQQQQQQLKHQQQQKQHQQQQQQQQ |
| | | | QQQQQQQQQQQQQQQQQQQKQQQQQQQQQQQQQQQQQQQQQQQQQQQQQQQQQQQQQQQQQ$QHIKQQIKQQ |
| 345 | F | 0.87 | # WFVFFFFFFFFFFFFFFRFFFYFFFFFFFKYFFFFFFFFFFFFFFFFFFFFFFFFFFFFFFFFFFFFFFFFFFFFFFFFFFF |
| | | | FFFFFFFFFFFFFFFFFFFFFYFFFYYYFFFFFFFFFFFFFFFFFFFFFFFFFFFFFFFFFFFFKFFFKFFF |
| 346 | G | 0.474 | # --KRKGK-KGKGGEGGGQKGGKNGGRGVEQERRGGG-K-EG-GGGVRVGEKKEGGGGREVKVKVKE-VRGGGRGGGGGEGEG |
| | | | GGGGGGGEGGGGGGGGGEKGGGKGVKKK-GKGGGGKDGGKRRGEKGNGGEGGEGKGGGEGKRGKQR-GQEG- |
| 347 | N | 0.939 | # NNNNNYNNKNNNNNNNNNNNNNNNNNNNNNNNNNNNNNNNNNNNNNNNNNNNNNNNNNNNNNNNNNNNDNNNNNNNNNNNNN |
| | | | NNNNNNNNNNNNNNNNNNNNNNNNNNNNNNNKNNNNNNNNNNNNNNNTNNNNNNNNNNNNNNNNNNNNNNNN |
| 348 | K | 0.819 | # QKKKKKKKNKKKKKKTKKKKKKKKKKTKKKKKKKKKRKKSKNKKRKKKKKVKKKKKKKKKAKKKKKKKKKSKKKKKKKRKKK |
| | | | KKKKKKKKKKKKKKKKKKKNKKKKKKKKKNKKKNKKKKKKKKKKKKKKIKKNKKKKKKKKKKKKKKKKKKKK |
| 349 | T | 0.899 | # TTTTTTTTTTTTTTTKTTTTTTTTTTKTTTTTTTTTTTTTTSTTTTTTTTTTTTTTTTTTTTTTTTTTATNTTTTTTTTTTT |
| | | | KTTTTTTTTTTTTTTTTTTNTTTTETTTTKTTTTTTTTTTTTTTTTTTTTTTTTTTTTTTTTTTTTTTTTTT |
| 350 | I | 0.968 | # IIIIIIIIIIIIIIIIIIIIIIIIIIIIIIIIIIIIIIIIIIIIIIIIIIIIIIIIIIIIIIIIIIIIIIIIIIIIVIIIII |
| | | | IIIIIIVIIIIIIIIVIIIIIIIIIIIIIIIIIIIIIIIIIIIIIIIIIIIIIIIIIIIIIIIIIIIIIIII |
| 351 | V | 0.661 | # IVAIVVVAVVVVIVVVVVVINAIIVVVVVVVAVVVVAVVVFVAVVVVVVSVAVIIVIVVVVAAAVAIAAAVVVKVVVVIVVV |
| | | | VVIVVAVVVVVVNVVVVVVIVVVAEIIIIISVVVIVVVAVVIIIVVVVVVIIVVIVVIVVVVVVVAVVVVVV |
| 352 | F | 1 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 353 | N | 0.754 | # DNKNNNNKNNKKNNNNNNNKNRRKNNNNNNNDKNNNNNNNNNNKKNNNTNKNNNNNNNNNNNKNNKKNNKKNNNNNNNNNNK |
| | | | NNNNNNNNNNNNSKNNNNQNNNNNKNNNNKKKNNNNTNNNNNNNNNNNNNNNNNNNNNNNNRNNNNTNKNNK |
| 354 | Q | 0.615 | # HQQHHQQPPPPQQQQHQRQQQPNPQHQQQRHRPRRQRQQQQQQQNQQHQQSQQHQQQQQRHQKQRQSQQQQQQQQQQQQQQQ |
| | | | QQQQQQQQQQQQQQQQQQPQQHRQGQQQRQQQQQSHSQQQQQQRQQHQHHQHHQQRQPQQQQQHHQNQQKHN |
| 355 | S | 0.911 | # PSASSSSSSSSSSSSSSSSSSSSSSSSSSSSSSSSSSSSSPSSSSSSSSPSPSSSSSSSSSSSSSSSSSASSSSSSSSSSSS |
| | | | SSSSSSSSSSSSPSPSSSSSSSSSSSSSSSSSSSSSSSSSSSSSSSSSSSSSSSSSSSSSHSSSSSSSSSSS |
| 356 | S | 0.905 | # SSSSSSSSSSASSSSSSSSSSSSSSSTSSSSSSSSSSSSSSSSSSSSSAASSASSSSSSSSSSSSSSSSSSSSSSSSSSSAS |
| | | | SSSSSSSSSSSSSSSSSSSSSSSTSSSSSSSSSSSSSSSSSSSSSSSSSSSSSSSVSSSSLSSSSSSSSSSS |
| 357 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 358 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 359 | D | 1 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDD |
| 360 | P | 0.83 | # LLPPPPPPPPPPPLPPPPPPPPPPPPPPPPPPPPAPPPPPPPPPPPLPPPPPPPPPPPPPLPPPPPPPPPPPPPPVIPVPPP |
| | | | PVPPPPVPPVIPPVVILPPPPPPPPPPPLPPPPPPPPPPPPPPPPPPPPPPPPPPLPPPPPPPPPPPPPPPP |
| 361 | E | 0.987 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEKEEEEEEEEEEEEEEEEEEEEEEEEEEEEEE |
| 362 | I | 0.93 | # IIIIIIIIIIIIIIIIIIIIIVIIIIIIVIIIIIIIIIIIIIIIIILIIIIIIIIVIIIILIIIIIIIIIIIIIIIIIIIII |
| | | | IIIIIIIIIIIIIIIIIIIIIIIIIIIIIIIVIIIIIIIIIIIIIIIIIIIIIIIIIIIIVIIIIIIIIIII |
| 363 | V | 0.883 | # VVVVVVVVVVKVVTVVVVVVVVVVVVVVVVVVVVVVVVVVVVEIVVVVVVTVVVVVVVVVVVVVVEVVVVVVVVVVVVVVVV |
| | | | VVVVVVVVVVVVVEVVVVVTVVVVVVVVEVVVVVVVVVVVVVVVVVVVVVVVEVVVVVVVVVVVVVVVVVVA |
| 364 | M | 0.731 | # MMMMRTMTMMMMMMMMTMMTMTMMMMMMMMMMTMMMMMMMRMMMTTMMMMTMMTTLMMMMMMLMMMTMMMMMMRRMTMMLLM |
| | | | MMMLMMMMMMTMLMTTMMTMMMMMMMMMMTMMMMFTLMMMMMMMTTMMMMTTTMTTMTMTTMMMMMTMMMMM |
| 365 | H | 0.961 | # HHHHHHHHHHHHHHHHHHHHHHHHHHHLHHHHHHHHHHHHHHHHHHHHHHHHHHHHHHHHHHHHHHHHHHHHHHHHHHHHHH |
| | | | HHHHHHHHHHHHHHHHHHHHHHHHHHHHHHQHHHHHHHHHHHHHHHHHHHHHHHHHYHHHHHHHHHHHHHHH |
| 366 | S | 0.845 | # TITSSSSSSISSSTSSSSSSSSSSSSSTSSSSSSSSSSSSTSSTMSTSSSSSISSISSNSTSSSSSSSSSSSSSTSSSSSST |
| | | | SSSSSSSSSSSSSSSSSSSTSSSSSSSSSSSSSSSSSSSSSSSSSSSTSSSSSSSSSSSSSSSSSSSSSSSS |
| 367 | F | 0.964 | # FFFFFFFFFFFFFFFFFFFFFFFFFFFFVFFFFFFFFFFFFFFFFFFFFFFFFFFFFFFFFFFFFFFFFFFFFFFFFFFFFF |
| | | | FFFFFFFFFFFFFFFFFFLFFFFFVFFFFFFFFFFFFFFFFFFFFFFFFFFFFFFFFFFFFFFFFFFFFFFF |
| 368 | N | 0.929 | # NNNNNNNNNNNNNNNNNNNNNNNNNNNNNNNNNNNNNNNNNNNNNNNNNNNNNNNNNNNNNNNNNNNNITINNNNNNNNNNN |
| | | | NNNNNNNNNNNNNNNNNNNNNNNNNTTTNNNNNNNNNNNNNNNNNNNNINNNNNNNNNNNNNNNNNNNNNNN |
| 369 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 370 | G | 0.911 | # GGGGGGGGGGGGGGGGGGGGGGGGGGGGGGGGAGGGGGGGAGGAAGGGGEGGGGGGGGGGGGGGGGGGGGGGGGGGGGGGGG |
| | | | GGGGGGRGGGGGGGGGGGGGGGGGGGGGGGGGGGGGGRGGGGGGGGGGGGGGGGGGGGGAGGGGGGAGGGGG |
| 371 | G | 0.987 | # GGGGGGGGGGGGGGGGGGGGGGGGGGGGGGGGGGGGGGGGGGGGGGGGGGGGGGGGGGGGGGGGGGRGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 372 | E | 1 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 373 | F | 0.974 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFLFFFFFFFFFFFFFFFFFFFFFFFFFFFFFFFFFYFFFFFFFFF |
| 374 | F | 0.956 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFLFFFFFFFFFFFFFFFFFFFFFFFFFFFFFFFFFFFLFFFFFFFFFLFFFFFFFFFFFFFFFF |
| 375 | Y | 0.94 | # YYYYYYYYYYYYYYYYYYYYYYYYYYYYYYYYFYYYYYYYYYYYYYYYYYYYYYYYYYYYYYHYYYYYYYYYYYYYYYYYYY |
| | | | YYFYYYYYFYYYYYYYYYYYYYFFYYYYYYYYYYYYYYYYYYYYYYYYYYYYYYYYYYYYYYYYYYYYYYYY |
| 376 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 377 | N | 0.847 | # NNDNNNNNDNNNNNNNNNNNDNNNDNNNNNDNNNNNNDDNNNNNNNNNNNNNNSDNNKDNNNNNNNNNNNNNNDNNNNNNNN |
| | | | SNNNNNNDNNNNNNNNNNNNNNNNNDDDNNNNNNNNNNNNKNNNNNNNNNNNNNNNNNKNNNNNNNNNNNNN |
| 378 | T | 0.751 | # TTTSSSSSSSTSTSTTSSSSTSTTSTTTTTSTTTTTTSSTTTTTTTSSSTTTTTSTTTSSTTSTTTTTTTTTTTTSTSTTAA |
| | | | TSTTTTTATTTSTTTTATSTSSTTTTTTTSTTSSTSTTSTTTTSSTSSTSSTTTSSSSSTSSSSSTTTSTTT |
| 379 | T | 0.809 | # TTTTTTTTTTTTTTSTATTTTTTTTTTTATTSTTTTTSSSTTTKTKTTTTTSTTSTTASTTTTTTTSTTTTTTTSTTTTTTT |
| | | | TTTTTTITTTTTTTTTTTTTTTTTTTTTTTTTTTSTTTTSTTTTTTTTSTTTTAATTTTTTTTTTATTTKTT |
| 380 | Q | 0.652 | # KQKQQQQQRQPKEQQKQKQQQKQGQQQRQQQQQKKQKQQQKQKQKQQPQQKQPEQQQQQQQKQKQQKKQNQGQQQQKQKQQP |
| | | | EQKIQQQQQQQQKQQKPKPQQQQQQKKKKQKNQQQQQQQPQKKQQQQQQQQKQKQQKQQKKPQQQQQQQKQK |
| 381 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 382 | F | 0.987 | # FFFFFFFFFFFFFFFFFFFFFFFFFFFFFFFFFFFFFFFFFFFFFFFFFFFFFFFFFFFFFFFFFFFFFFSFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 383 | N | 0.907 | # NSNNNNNNNNNNNNNNNNNNNNNNNNNNNDNNNNNNNNNNNNN-NNNSNNNNNNNNNNNNNNNNNNNNNNNNNNNNNNNNNN |
| | | | NNNNNNNNNNNNNNNNNNNNNNNNNNNNNNNNNNNNNNNNNNNSNNNNNNNNNNNNN-NNNNNSNNDNNNND |
| 384 | S | 0.811 | # SSSSSSGGSSRSSSSSSSSSSSSSSSSSSSSSSSSSSSGSSSS-SSSGSSSSSDSSTSSSSSSSSSSSSGSSSSGSSSGSSS |
| | | | SSNSSSSSSSSSTSSSSSSSSSSSISSSSSSSSSSSSNSSSSSSSSSSSSSSSSSSR-SSSSSSSSSSSSSK |
| 385 | T | 0.895 | # TTTTTTTTTITTTTTTTTTTTTITTTTTTITTTTTTTTTITTTNITTTTTTTTTTTTTTTTTTTTTTTTTTTTTTTTTTTTS |
| | | | TTTTTTTTTTTTTTTTTTITTTTTTTTTTTTTTTTTTTATTTTTTTTTTTTTTTTTTTTTTTTTTTITTTTI |
| 386 | W | 0.926 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWAWWWWWWWWWLWWWWWWWWWWWNWWWWWWWWWWWWWWWW$WWWWWWWWWWWWWWWWWWWWWWWWWWWWWW |
| 387 | N | 0.923 | # NNGNNNNNNNNNNNNNNNNNNNNNNNNNNNNNNNNNNNNNNNNNNNNNNDNNNNNNNNNNHNNNNNNNNNSNNNNNNNNNNN |
| | | | NNNNNNTNNNNNNNNNNNNNNNNNNNNNNNNNNNNNNNNNNNNNNNNNNNNNNNNNNNNNNNNNNNNENNNN |
| 388 | G | 0.362 | # GELGSSDGGDGDFGDGDNGSVGNSNDSATDSDDSSVESDSDGDDGSGTGDGEGNSGGSGSDEDESSQEIGDGDGNDSGSDGG |
| | | | DDDDGWDHGSSDDGDSADIGGSTNTSSSESDGDGIGSHVNSNNGSGSVSNSNSSDSSDNSNWNNSVGGGGGG |
| 389 | T | 0.843 | # TTNTTTTTTTTTTTTTTTTTTTTTSITSETTTTTTSTTTTTTTTTTTSTATTTTTATTTTVTTTTTTTTTGTTTTTTNTTTT |
| | | | TTTTTTTTTTTTTTTTTSTTTTTTTTTTTTTTTTTTTTTTTTTTTTTTTTTTTTTTTTTTTSTTTTTSTTTT |
| 390 | W | 0.407 | # LWGAGWWWRWWWW-WW-WWWWMWKW-GNLNWRVWWLGWGKKWSAWGRWGWWN--WKWGWW-WTWWWGW-WWVGGIRW-W-W- |
| | | | WW-WMRK-WWWRNWWWLWGWWWGWGWWW-WL-WWVKGGSWW$WGGWLRGEGGGWEWWRWWWPGGGGWGWNWW |
| 391 | N | 0.837 | # NNNNNNNNNNNNNNNGNNNNNN--NNNNNNNNNNGNNNNNNN--N-NNNNN-NNNNNNNNNNNNNNNNNNNNNNNNNNNNNN |
| | | | NN-NNNNNTNNN-NNNNNNNNNNN-NNNNNNFNNNNNNGNNNNNNNNNNNNNNNNNNNSINNNNNNNNGNNN |
| 392 | D | 0.304 | # NESGDDNGENTDMGDSNDNNED--IRTSKDSSNTDIIDGIND--S-NDNSG-DSDEDGGNTSDSKSDSTGERDNSDGGDGGT |
| | | | TD-NEFDIVDDD-GDDANNDGIIS-DDDGNNDENKSNDTNDNDNNDDNINDNNNNDDNDAININSGNSADGN |
| 393 | T | 0.759 | # TTTTTTDTTSTTTTTSTTTTTT--TTTTTTTTTTDTTTTTTT--T-TTTST-TTTTTTTTETTTTTTTTTTTTTTTTTTTTT |
| | | | TT-TTTTRSTTT-TTTTTTTTTTT-TTTKTTSTNTTTTWTTTTTTTTSSTTTTTTTTTDTTDTTTTTTDTTT |
| 394 | E | 0.288 | # ANKQEGTWETDTSKTD--EEGE--ETTTK-EEKE-EDD-KGK--D-EVGEW-E-DTEIGG-EEKDE-EEWKGKE-RRGGTNG |
| | | | EW-TTEGS-RRT-GWRNEKEEEEE-TTTGEIT-TKLG-STEKEGEGSTEETEEEREEGTEEVKEWEG-N-EE |
| 395 | G | 0.374 | # GT-LRE-KGWIGGGGT--GGEG--GG-EG-IVGE-RIT-GSG--T-GGGGN-G-G-GKPG-KGEKR-KR-EEAG-EEGEEGS |
| | | | S--EGSEE-GKG-SKEEGGGGGGG-GGGEGGL-DE-G-VGR-DGEGTESGGGGGEGGRGGR-EGSGR---GE |
| 396 | N | 0.66 | # NN-NNN-NNG--NNN-N-N-NN--NN--N---N--N-N--N---N-NNNNN-N-R-NN-NN--NNN--NT-N-N---NN-N- |
| | | | NK-N-N--NNN---D--DNNNN-N-GGG--N--NN-N-KSNNGN-NNNN-NNNNNNN--NN--N--N---NN |
| 397 | I | 0.698 | # IIIIILIIIIIIIIIIIIIIIIIIILIIIIIIFIIFIIIIIIIIIIIIIIIIIIIIIIIIIIIIIIIIIIILIIIIIIIIII |
| | | | IIIIIINNIIIIIIIIIVIIIIIIMIIIIIIIIIIIIIIIIIIIIIIIIIIIIIIIIIIIIIIIIIIIIIII |
| 398 | T | 0.81 | # ITTTTTTTTTITTTTTTITTTMTITITTTTTTTTTTTTTTITTTRTTITITTTTTITTTTTTITTTTTTTTITTTTTTTTTT |
| | | | TTTTTTIIITTTTTTTTTITTTTTTTTTTTTTTTITTITTITTTTSTTTTTTIITTTTTTTTTIRTTETTTT |
| 399 | L | 0.977 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 400 | P | 0.804 | # PQPPPPPPPPPLSPPPPPPPPTPPPPPPPPPPPPPPPQQPPPPPPPPQPQPPKPQPPPPPPQPQPPPQQSPQPPPPPPPPPL |
| | | | PPPPPPPPPPPPPPPPLPTPPPPPPPPPPPPQPPPPPPPQPPPPPPPPPPPPPPPPPPPPPPPLPQPPPPPP |
| 401 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 402 | R | 0.933 | # RRRRRRKRKRRRRRRRRRRRRRRRRRRRRRRRRRRRRKKRRRRRRRRRRRRRRRRRRRRRRRRRRRRRRRRKRRRRRRRRRR |
| | | | RRRRKRRRRRRRRRRRRRRRRRRRRRRRRRRRRRRRRRRKRRRRRRRRRRRRRRRRRRRRRRRRRRRRRRRK |
| 403 | I | 0.987 | # IIIIIIIIIIIIIIIIIIIIIIIIIIIIIIIIIIIIIIIIIIIIIIIIIIIIIIIIIIIIIIIIMIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 404 | K | 0.89 | # KKRKKKKKKKKKKKKKKKKKRKKKKKKKRKKKKKKKKKKKKKKKKKKKKKKRKKKKKKKKKKKEKKKKRKKRKKRKKKKKKK |
| | | | KKKKRKKKKKKRKKKKKKKKKKKKKKKKRKKKKRSKKKKKKKKKKKKKKKKKKKKKKKKKKKKKKKKKKKKK |
| 405 | Q | 0.974 | # QQQQQQQQQHQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQEQQQQQQQQQQQQQQQQQQQQQQQQQQQQ |
| 406 | I | 0.923 | # IFIIIIIIIIIIIIIIIIIIIIIIIIIIIIIIIIFIIIIIIIFIIIIIIIIIFIIIIIIIIIIIVIIIIIIIIIIIIIIIII |
| | | | IIIIIIIIIIIIIIIIIIIIIIIIIIIIFFIIIIIIIIIIIIIIIIVIIIIIIIIIIIIIIIIIIIIIIIII |
| 407 | I | 0.823 | # IIIIIVIIIVIIIIVIIIIIIVIIVIIIIIIVVIIIIIIIIIIIVIIIIIVVIIIIIVVIVIVIVIIIIIIVIIVVIVIIIV |
| | | | IVIIVVIVIIIVIIVIIIIIIIIIIIIIVIIIIIIIVIIIIIIIVIVIIIIIIIIIIIIIIIMVIIIIIIII |
| 408 | N | 0.961 | # NNNNNNNNNNNNNNNNNNNNNNNNNNNNNNNNNNNNNNNNNNNNNNNNNNNNNNNNNNNNTNNNNNNNNNNNNNNNNNNNNN |
| | | | NNNNNNNNNNNNNNNNNNNNNNNNNDNNNNNNNNNNNNNNNNNNNNNNNNNNNNNNNNNNNNNNNNNNNNNN |
| 409 | M | 0.737 | # RMMMMLRMLMMRMMMMMMMMRRMMMMMMMLMMMRRMLLLMRMMRMMMLMMMRMKMRRMMMMRLRMRMRRRRLMMMMMMMMMM |
| | | | MMMMMMMMMMMRMMMMMMMMMMMMMMMMMMMMMRMMMMRMRRRMMMTMLMMMMRMMMMMMMRMMMMRLRMMM |
| 410 | W | 0.943 | # WWWWWWWWWWWWWWWWWWWWWWWWWWWWWWWWWWWWWWWWWWWWWWWCWWWWWWWWWWWWWWWWWWWWWWWWWWWWWWWWWW |
| | | | WWWWWW$WWWWWWWWWWWWWWWWWWWWWWWWWRWCWWWWWWWWWWWWWWWWWWWWWWWWWWWCWWWWWWWWW |
| 411 | Q | 1 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 412 | E | 0.721 | # EKEEKEEEEEKEEEEEEEEKEEEEEEKEEEEEKEEEGEEEGEEEEEEKEEEEEEQEEEEEKEEEEEKEEEEQEGERREKEEE |
| | | | EKEEEEREERREEKKRREEKEEEEEEEEKKEEEEEGEEVEEKEEEEEGEEKEGEEEEEEEGEEEEKEEEEEG |
| 413 | V | 1 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 414 | G | 0.974 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGREGGGGGGGGGGGGGGGGGGGGGGGGGGGGG |
| 415 | K | 0.968 | # KKKKKKKKKKKKKKKKKKKKKKKKKKKKKKKKKKKKKKKKKKKKKKKKKRKKKKKKKKKKKKKKKKKKKKKKKKKKKKKKKK |
| | | | KKKKKKKKKKRKKKKKKKKKKKKKKKKKKKKKKKKKKKKKKKKKKKKKKKKKKKKKKKKKKKRKKKKKKKKK |
| 416 | A | 0.987 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAPAAAAAAAAAAAAAAAAAAAAAAAAAAAAAAAAAAAAA |
| 417 | M | 0.974 | # MMMMMMIMMMMMMMMMMMMMMMMMMMMMMMMMMMMMMMMMMMMMMMMMMMMMMMMMMMMMMMMMMMMMMMMMMMMMMMMMMM |
| | | | TMMMMMMMMMMMMMMMMMMMMMMMMMMMMMMMMMMMMMMMMMMMMMMMMMMMMMMMMMMMMMMMMMMMMMMM |
| 418 | Y | 1 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 419 | A | 0.987 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAVAAAAAAAAAAAAAAAAAAAAAAAAAAAAAAAAAAAAAAAAAAAAAAAAAAAAAAAAAAAAAAA |
| 420 | P | 0.987 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPLPPPPPPPPPPPPPPPPPPPPPPPPPPPPPPPPP |
| 421 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 422 | I | 0.953 | # IIIIIIIIIIIIIIIIIIIIIIISIIIIIIIIIIIIIIIIIIIIIIIIIIIIIIIIIIIIIIIIIIIIIIIIIIIIIIIIII |
| | | | IIIIIIIIIIIIIIIIIIIIIIIIISSSSIIIIIIIIIIIIIIIIIIIIIIIIIIIIIIIIIIIIIIIIIII |
| 423 | R | 0.501 | # SRSRNSRRRRRERKERSGRSKRKRRRRRARRSRSSKQRRRKSRESKRRRRSKRRRSTTREEERERRSERASQKSRRRRRRRR |
| | | | RRRRRRRRRRKRRKRREARSRRRRRSSSDSRRARRQSGKESRRKEERRRRRRGRRRKRSASSKRDTKRESKR |
| 424 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 425 | Q | 0.663 | # VHQQQLQQQQIHQQQQQLQQQQQKQQQQQQQQLQQKQRRNPQQQRQQQINQLQQQYTQQQKIQIQQRIQKLQQQQQQYQQQQ |
| | | | QQQQLQQQQQQQQQQQLQQQQQQEQQQQQQQHQQQQQQLQQQQQQQQQQQQQQQQQQQQQLQQQQQNQQQQN |
| 426 | I | 0.964 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIVIIIIIIIIIIIIIIIIIIIISIIIIIIIIIIIIIVIIIIIIIIIIIIIIIIIIIIIIIIIIII |
| 427 | R | 0.703 | # SRNRRRKKRRNRKRRRRRRRRRRKRRRRRRRRRRRRSRRSRRRRRRRRSSRRRKRNSRRRSRRRSRARRSRSRRRRRNRRRT |
| | | | RRRRSRRRRRRSRRRRKRKSRKKRRRRRRRRRRRIRKSRRRKRKRRRRRRRRRRKRRRRRSRRRRRSRRWSS |
| 428 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 429 | S | 0.732 | # VSSSSSSTTTTSSVSSSSSSSSSSSSLSVSSSPSLSSSSSSSSSVSSSSLSTSTTSSASSLSSSSSSSSSSSSSSSSSASSS |
| | | | SSSSSSASSAASSASSSSVSSSSSKSSSTSSVSSSSISSSSSSSSSSSSSSSSSASSSTSSSSSSSSSLSSS |
| 430 | S | 1 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 431 | N | 0.91 | # NNNNNNNNNNNNNNNNNNNNNNNNNNNNNNNKNNNNNNNNNNNNNNNKNNNNNNNNNHNNNNNNNNHNNNNNNNNNNNNNNK |
| | | | NKNNNNNNNNNNNNKNNNNNNNNNNNNNNNNNNNNNNNNNNNNNNNNNNNNNNNNKNNNNNNKNNNNNNNNK |
| 432 | I | 1 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 433 | T | 1 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 434 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 435 | L | 0.918 | # LLLLLLLLLMLLLLLLLLLLLLLLLLILLLLLILLLLLLLLLLLLLLLLLLLLLLLLLLLLLLLLLLLLLLLLLLLLLLLLL |
| | | | LLLLVLLLLLLVLLLLLLLLLLLLLLLLLLLLLLLLLLLLLIILLLLLLLLILLLLLLLLLLLLLLLLLLLL |
| 436 | L | 0.88 | # LLLIILLLILILLLLLLIILLILLLLLLLLLLLLLLLLLLLLLLLLLLIILLLVIILLLLLLLLLLLLLLLLLLLLLLLLLL |
| | | | LILLLLLLLLLLLLLLMLLLILLLLLLLLLLLLLLLLLLLLLLLLLLLLLLLLLLLLLLLLLLLLLLLLLLL |
| 437 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 438 | T | 0.949 | # ATTTTTTTTTTTTTTTTTTTTTTTLTTITTTTTTTTTTTTTTTTTTTTTTTTTTTTTTTTMTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 439 | R | 0.974 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRHRRRRRRRRRRRRRRRRRRRRRKRRRRRRRRRRRRRRRRRRRRRRRRRRRRRR |
| 440 | D | 0.964 | # DDDDDDDDDDDDDDDDDDDDDGDDDDDDDDDDDDDDDDDDDDDDDDDDDDDDDDDDDDDDDDDDDDDDDDDDDDDDDDGDDD |
| | | | DDDDDDDDDDDDDDDDDDDDDDDDDDDDDDDDDDDDDDDDDNDDDDDDDDDDDDDDDDDDDDDDDDDDDDDD |
| 441 | G | 0.987 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGVGGGGGGGGGGGGGGGGGGGG |
| 442 | G | 0.946 | # GGGGGGGGGGGGGGGGGGGGGGGGGGGGGGSGGGGGGGGSGGGGGGGGGGGGGGGGGGGGGGGGGGGGGGGGGGGGGGGGGG |
| | | | GGGGGGGGGGGGGGGGGGGVGGGGGGGGGGGGGGGGGGGGGSGGGGGGGGGVGGGGGGGGGGGGGGGGGGGG |
| 443 | N | 0.291 | # GI-DNKPN-NLKDGN-KNINADGNNN-NN-GKKSSGNDNNNKNSN-RT-ITNH-DTRRNDNT-T-T-TI-NNNKNTSISNNN |
| | | | TTN-TVT-N-N-N-IST---TGNNEIIIGNEMNNT-ETGKDTTN-STNSILTKTNTNKTNN-TNN-K-SKM- |
| 444 | N | 0.961 | # NNNNNN-NNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNKVNNNNNNNNNNNNNNNNNNNNNNNNNNNNNNNNNNNNNNNNNNNNNNNNNNNN |
| 445 | E | 0.27 | # TTDNKD-TNGSERSENESQEAETNLENDEE-TGKEEEDDGSNKNESEKNESVGEKNENNEKEIETEDEEEGNGKGGNGNTNG |
| | | | NGNNEGKDNDSHNSGSDQDGSGEAHGGGKNEGDNRSTVQDSNNGSKGERGDGDENGEGTSGTGGESSNQSKE |
| 446 | T | 0.679 | # STTSTT-TTTTSSTSTTSTSTTSTTTTTTSETTTTSTTTTSTTTTSTNSSTTTTSTTTTTTTTTTTTTTTTSTTSTSTSSTT |
| | | | TTPSTTTTSSTTTSTTSSELSSTNTSSSTGTTSTTSTTTTTSSSSSTSTTITTTTNTTTSTTSSTTRSTSTS |
| 447 | E | 0.939 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEENEEEEEEEEVEEEEEEEEKQEEEEEEEEEEEKEEEEEEEEEEEEEEEEEEEEEEEEEEEEEE |
| 448 | I | 0.722 | # TTIIITITITTTTTTTTIIITTTTIIIIIITIIIITTIIITITIITIITTTITIIITTITIIIITITIIITIIITTTITIIT |
| | | | ITIIITTIIIIIIITTVITTIVIIITTTTIVVIITIIVIIITTIIIITIIITTITITITIIIFVIITIIIIT |
| 449 | F | 1 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 450 | R | 0.987 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRGRRRRRRRRRRRRRRRR |
| 451 | P | 0.987 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPLPPPPPPPPPPPPPPPPPPPPPPPPPPPPPPPPPPP |
| 452 | G | 0.891 | # VGGGGGGGGGGGGGGGGGGGGGGGGGGGGGGAGGGGGGGGGGGGGGGGGVGIGGGGGGGGGGGGGGGGGGEGGGGGGGGGGG |
| | | | GGGGGGGGGGGGGGGGGGQGGGGTGGGGGGGGGGGGGGGGGKGGGGGGGGGGAGGGGGGGGGGGGGGGGGGG |
| 453 | G | 0.974 | # GGGRGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGEGGGGGGGGGGGGGGGGGGGGGGGGGGGGGG |
| 454 | G | 0.977 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGEGGGGGGGGGGGGGGGGGGGGGGGGGGGGGG |
| 455 | D | 0.824 | # DDNDDDDDDDDDNNDNNNDDDNDDDDDDDDDDDDDDNDDDDDDNDDNDDDNNDDDNDDDNNDDDDDNDNDNDDDNNDDDNDD |
| | | | DNDDDDDNDDNDDNNDDDNNDDNDDDDDDDDDDDNNNDDDDNDDDDDDDDDDDDDDDDDDNDDDDDDDNDDD |
| 456 | M | 0.968 | # MMMMMMMMMMMMMMMMMMMMMMMIMMMMMMMMMMMMMMMMMMMMMMMMMMMMMMMMMMMMMMMMMMMMMMMMMMMMMMMMMM |
| | | | MMMMMMMMMMMMMMMMMMMMMMMMMMMMMMMMMMMMMMMMMIMMMMMMMMMMMMMMMMMMMMIMMMMMMMMM |
| 457 | R | 0.854 | # RRKKRRRRRRRRRKRKRRKRRRRRRRRRRRRRRRRRRRRRKRRKRRKRRRKRRRRKRRRRKRRRRRRKKRKRRKRKRRRRRR |
| | | | RKRRRRRRRRRRRKKRKRRRRRRRRRRRRRRRRRKRRRRRRRRRRRRRRRRRRRRKRRRRKRRRRRRRKRRK |
| 458 | D | 0.977 | # DDDDDDDDDDDDDDDDDDDDDDDDDNDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDNDDDDDDDDDDDDDDDDDDDDDDDDDDDDDD |
| 459 | N | 0.987 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNINNNNNNNNNNNNNNNNNNNNNNNNNNNNNNNNNNNNNNNNNNNNNNNNNNNNNNN |
| 460 | W | 0.987 | # WWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWW$WWWWWWWWWWWWWWWWWWWWWWWWWWWWWW |
| 461 | R | 0.964 | # RRRRRRRRRRKRRRRRRRRRRRRRRRRRRRRRRRRRRRRRRRRRRRRRRRRRRRRRRRRRRRRRRRRRRRRRRRRRGRRRRR |
| | | | RRRRRRRRRRRRRRRRRRRRRRRRRRRRRRRRRRRRRRRRRKRRRRRRRRRRRRRRRRRRRRRRRRRRRRRR |
| 462 | S | 0.964 | # SSSSSSSSSCSSSSSSSNSSSSSSSSSSSSSSSSSSSSSSSSSSSSSSSSSSSSSSSSSSSSSSSSSSSSSSSSSSSSSSSS |
| | | | SSSSSSSSSSSSSSSSNSSSSSSSSSSSSSSSSSSSSSSSSSSSSSSSSSSSSSSSSSSSSSSSSSSSSSSS |
| 463 | E | 0.948 | # EEEEEEEEEEKEEEEEEEEEEEEEEEEEEEEEEEEEEEEEEEEEEEEEEEEEEEEEEEEEEEEEEEEEEEKEEEEEEEEEEE |
| | | | EEEEEEKEEEEEEEEEEEEEEEEEEEEEEEEEEEEEEEEEEKEEEAEEEEEEEEEEEEEEEEEEEEEEEEEE |
| 464 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 465 | Y | 0.987 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYCYYYYYYYYYY |
| 466 | K | 0.953 | # KKKKKKKRRRKKKKKKKKKKKKKKKKKKKKKKKKKKKKKKKKKKKKKKKKKKKKKRKKKKKKKKKKKKKKKKKKRKKKKKKK |
| | | | KKKKKKKKKKKKKKKKKKKKKKKKKKKKKKKKKKKKKKKKKKKKKKKKKKKKKKKKKKKKKKKKKKKKKKKK |
| 467 | Y | 1 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 468 | K | 1 | # KKKKKKKKKKKKKKKKKKKKKKKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 469 | V | 0.956 | # VVVVVVVVVVVVVVVVVVVVVVVIVVVVVIVVVVVVVVVVVVVVLVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVIVVVVVVVV |
| 470 | V | 0.953 | # VVVVVVVVVVVVVVVVVVVVVVVVVVVVVVVVVVVVVVVVVVVVIVVVVVVVVVVVVVVVIVVVVVVVVVVVVVVVVVVVVV |
| | | | VVVVVVVVVVVVVVVVVVVIVVVVVVVVIVVVVVVVVVVVVVVVVVVVVVVVVVVVVVVVVVVVVVVVVVVV |
| 471 | K | 0.749 | # RRQKRKKQKKEKKKKKKKKKQKKKKKKKKKKKKKKRKKKKRRKKKKKKQKKKKKKKKKKKERKRKKKRKKRKKKKKKEKKKK |
| | | | QKQQKKKRKKKKQKKKKKKKKKKRKRRRKKKKKRRRRKKTKKKKKKKKKKKKKEKKKKKKKKKKKRKQKRKK |
| 472 | I | 0.93 | # IIIIIIIVIIIIIIIIIIIIILIIIIIIIIIIIIIIIIIIIIIIIIIIIIIIIIIIIIIIIIIIIIIIIIIIIIIIIIIIII |
| | | | IIIIIIIIIIIIIIIIVIIVIIIIIIIIIIIIIIIIIIIIIIIIIIIIIIIIIIIIIIIIVIIIIIIIIVIL |
| 473 | E | 0.946 | # EEEEEEEEEEEEEEEEEEEEKEEEEEEEEEEEEEEEEEEEEEEEEEEEEEEEEEEEEEEEEEEEEEKEEEEEEEEDEEEEEE |
| | | | EEEEEEEEEEEEEEEEEEEEEEEEEEEEEDEEEEEEEEEEEEEEEEEEEEEEEEEEEEEEEEEEEEEEEEEE |
| 474 | P | 0.987 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 475 | L | 0.933 | # VLIILLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLILLLLLLILLLLLLLLLLLLLLLLLLLLLLLLLLLLLLLLLLLLLLLLLLLLLILLLLLI |
| 476 | G | 0.964 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGKGGGGGGGGGGEGGGGGGGGGGEGGGGGGGG |
| 477 | V | 0.738 | # IVVIVVIIVIVVVVVVVVVVVIVVVVIIVVVVVVVVVAVVVVVVIVVLIVIIVVIVVVVVVLVLVIILIIILVVVIIVIIIV |
| | | | IIIIIVIVIVVKIVIILVVIVVVVVVVVVVVVIVVVVIVVVVVVVVVVVVVVVVIVVVVVIVVVVVVVVIVI |
| 478 | A | A | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 479 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 480 | T | 0.987 | # TTTTTTTTTTTTTTTTTTTTTTTTTTTTTTTTTTTTTTTSTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 481 | K | 0.807 | # KKKKKKKRKKKKRKKKKKKKRKKRKKKKKKKKKKRKKKKKKKKKKKKKKRKRRKRKKKRKRKKKKKKKKKRKKRKKKKKKKK |
| | | | KKKKTKKKKKKKKKKKRRKKKKKKKKKKKKRKKRQKRKKKKKKKRKKKKKKKRRKKKKTKKKRKKRKRKKRE |
| 482 | A | 0.974 | # AAAAAAAAAAAAAAAAAAAAAAAAAAAAAAAAAAAAAAAAAASAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 483 | K | 0.861 | # KKKKKKKKKKKKRKRKKKKKKKRKKRKKKKKRKKKKKKKKKKRKKKKKKKKKKRKKKKKKKRKRKRKRRRRKKKKKKKKKKK |
| | | | KKKKRKKKRKKRKKRKRKKKKKKKKKKKKKKKKKKKKKKKKKKKKKKKKKKKKKKKKKKKKKKKKKKKKKKK |
| 484 | R | 1 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 485 | R | 0.987 | # RRRRRRRRRRRRRRRRRRRRRRRRRRRRRRRRRRRRRRRRRRRRRRRRRRRRRRRRRRRRRRRRRIRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 486 | V | 0.987 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVTVVVVVV |
| 487 | V | 1 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 488 | Q | 0.961 | # QQQQQQQQQQQQQEQQQQQQQQQQQQQQQQQQQQQQQQQQQQQQQQQQQAQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 489 | R | 0.987 | # RRRRRRRRRRRRRRRRRRRRRRRRRRRRRRKRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 490 | E | 0.987 | # EEEEEEEEEEEEEEEEEEEEEEEEEEEEEEEEEEEEEEEEEEEEEEEEEEEEEEEEEEEEEEEEEEEEEGEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 491 | K | 0.956 | # KKKKKKKKKKKKKRKKKKKKKKKKKKKKKKKKKKKKKKKKKRKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 492 | R | 0.964 | # RRRRRRRRRRRRRRRRRRRRRRRRRRRRRRRRRRRRRRRRRRRRRRKRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 493 | A | 0.952 | # AAAAAAAAAAAAAAAAAAAAAAAAAAAAAAAAAAAAAAAAAASAAAAAAAAAAAAAAAAAA-AAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAA-AAAAA |
| 494 | V | 0.893 | # VIVVVVVVVVVVVVVVVVVVVVVVVVVVVVVVVVVAVVVVVIVIVVIVVVIVAVVVVVVVAVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 495 | G | 0.816 | # GGGGGGGGGG-GGGGGGGGGGGG-GTGGGGGGGGG--GGTG-GGGGGG-APGGGGGGGGGGGGGGGGGGG-GGGGGGGGGGG |
| | | | GGG-GGGGGG-GGGGGGGGGGG-GGGGGGGGGGGGGGG-GEGGGGGGGGGGGTGGGGGGGTGGGGGG-MGGG |
| 496 | I | 0.705 | # LILVIILIIILIIMIILLIILILMLLLLILILIIILLIILLLILIIILLLLIIIMLIIIIIILIILLIIIFIIIIIIIIIII |
| | | | IIIIMIILIIIMIIIIIIIIIIILLILLLVIIILILIIVLILTIILIIIIIIIIIILILIIIIIILILIIIL |
| 497 | G | 0.987 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 498 | A | 0.977 | # AAAAAAAAAAAAAAAAAAAAAAAAAAAAAAAAAAAAAAAAAAAAAAAAAAAAAAAAAAAAAAAAAAAAAAAVAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 499 | M | 0.686 | # MLLMMVLMVVMLMMMLVMMLMLMLMMMMLLVMVMMMMVVLLMVMVLMMMVLVVMMMLMVVVMLMLMMMMMLLMLMLMMMMMM |
| | | | MMMMLMMMMMMMMMMMLVMMMMLMMMMMMLVMMMLMMMMLLLLMLVMVMVMLMMVMVLLLVMVMMVMLMVMM |
| 500 | F | 0.933 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFLF |
| 501 | L | 0.933 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLFLLLFL |
| 502 | G | 0.987 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 503 | F | 0.968 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 504 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 505 | G | 0.952 | # GGGSGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 506 | A | 0.953 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAT |
| 507 | A | 1 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 508 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 509 | S | 1 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 510 | T | 0.987 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 511 | M | 0.987 | # MMMIMMMMMMMMMMMMMMMMMMMMMMMMMMMMMMMMMMMMMMMMMMMMMMMMMMMMMMMMMMMMMMMMMMMMMMMMMMMMMM |
| | | | MMMMMMMMMMMMMMMMMMMMMMMMMMMMMMMMMMMMMMMMMMMMMMMMMMMMMMMMMMMMMMMMMMMMMMMM |
| 512 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 513 | A | 0.987 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 514 | A | 0.855 | # AAAAAAAAAAAAAAAAAAAAAAAAAAAAAAAAAAAAAAAAAAAAAAAAAAAAAAAAVAA-VAAAAAAAAATAARAAAAAAAA |
| | | | AAAAAAAAAAAAAAAAAAAAAAAAAAAAAAAAAAAGGVTAAAATRAAAAAAAAAAAAAAAAAAAAAAAAAAA |
| 515 | S | 0.914 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 516 | M | 0.555 | # VVIILIIVLMIMVLIIMLMMMLLMIMLMMLLMMMMMMILMLMILIMIIIMVLLLVMVLMLVLVLMLMVLIIIIIILLLVILL |
| | | | ILIIMIILVLLMIMLLLIIVIVLMVIIILMVLIMVLILMVMVVMMLIMMMMMIMVMMIMLIILMMVVIMIII |
| 517 | T | 0.882 | # TTTTATTTTTTTTTTTATTTTTAMTTTTTTTTTTTATTTTTTTTTTTTTTATTTTTTTTTATTTTTTTTTTTTTTTTTTTTT |
| | | | TTTTTTTTTTTTTTTTTTTTTATTTAAATTATTTTTTTATATTTTTTTTTTTTTTTTTTTTTTTATTTTTTA |
| 518 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 519 | T | 1 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 520 | V | 0.96 | # VVVAVVVVVVAVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 521 | Q | 1 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 522 | A | 0.953 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 523 | R | 1 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 524 | Q | 0.73 | # QQQQQQQQQLQQQQQQQQQQQLQQQQQQLQLQLLLQLLLLQQLQLLQQQQLQQQQQQLQLKLQLQLQLLLLQQQQQRLLLLL |
| | | | LLLLLQLQLLLLQLPLQQQQLQLLLQQQQQQQQLQQHQQLQQQLQLLLLLLLLLLQLQQLQLLQQLQLLQQQ |
| 525 | L | 0.974 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 526 | L | 0.964 | # LLLLLLLLLLLLLLLLLLLLLLLLLLLLLLLLLLLLLLLLLLLLLLLLLLLLLLLLLLLLMLLLLLLLLSLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 527 | S | 0.955 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 528 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 529 | I | 1 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 530 | V | 0.987 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 531 | Q | 0.987 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 532 | Q | 1 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 533 | Q | 0.987 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 534 | N | 0.82 | # NNNNNNNNSINSNNRNNNNNNNNSNSNSNNNNNSSNNNNNNNNNNNNSSNNSNNSNNNNNNNNNSNNNNSNNNNRNNNNNDN |
| | | | NNNDSNNNNNNSNNNNNNSNNNNNSNNNNNNNNNNNNNNNNNNNNNNNNNNNNNNNNNNNNNNNNNSNNNSN |
| 535 | N | 0.961 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNN |
| 536 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 537 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLMLL |
| | | | LLLLLLLLLLLLLLLLLLLLLLLLLLLLLLLLLLLLLLLLLLLLLLLLLLLLLLLLLLLLLLLLLLLLLLLL |
| 538 | R | 0.916 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRKRRRRRRRTRRRRRRRRRRRRRRRRRRRRRRRRRRRRRRRRRRRRRRRRRRRRRRRRRRRRRRRRR |
| 539 | A | 1 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 540 | I | 0.987 | # IIIIITIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 541 | E | 0.922 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 542 | A | 0.974 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 543 | Q | 1 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 544 | Q | 0.987 | # QQQQQQQQQQQQQQQQQQQQQQQQQQQQQQQQQQQQQQQQQQQQQQQQQQQQQQQQQQQQQQQQQQQQQQQQQQQQQQRQQQ |
| | | | QQQQQQQQQQQQQQQQQQQQQQQQQQQQQQQQQQQQQQQQQQQQQQQQQQQQQQQQQQQQQQQQQQQQQQQQ |
| 545 | H | 0.96 | # HHHHHHHHHHHHHHHHHHHHHHHHHHHHHHHHHHHHHHHHHHHHHHHHHHHHHHHHHHHHHHHHHHHHHHHHHHHHHHHHHH |
| | | | HHHHHHHHHHHHHHHHHHHHHHHHHHHHHHHHHHHHHHHHHHHHHHHHHHHHHHHHHHHHHHHHHHHHHHHH |
| 546 | L | 0.869 | # LLLLLLLMLLLLMLLLLLLLLLLLLLLLLLLLLLLLLLLLLLLLMLMMLLLLMLLLLLLLMLLLLLLLMLLLLLLLLLLMLL |
| | | | LLLMLMLLLLLLLLLLMLLMLLMLLLLLLLLLLMLLLLLMLLLLLLLLLMLLLLLMLLLLLLLLMMLLLMLL |
| 547 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLFLLL |
| 548 | Q | 0.952 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQEQQQ |
| 549 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 550 | T | 1 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 551 | V | 0.968 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVIIIVVVVVVVVVVVVVVVVVVVVVVVVVVVVVVVVVVVVVVVVVVVV |
| 552 | W | 0.977 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 553 | G | 0.987 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 554 | I | 0.964 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 555 | K | 1 | # KKKKKKKKKKKKKKKKKKKKKKKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 556 | Q | 1 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 557 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 558 | Q | 0.977 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 559 | A | 0.974 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 560 | R | 0.977 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 561 | V | 0.856 | # VVVVIVLVVVVVLVVVVVVIVVVVVIIVIVVVVVVVVVVVVVVVIVVVVIVVVVIVVVVIVVVVVVIVVVVIVVVVVVVVVV |
| | | | VVVVVVVVVLLVVVVVVVVVVVVVVVVVVVVVVVVVVVVVVVVVVVVVVVVVVVVVVVVVVVVVVVVVVVVV |
| 562 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 563 | A | 0.974 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 564 | V | 0.923 | # VVIVVLVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVLVVVVVVVVVVVVVVVVVVVVVVVVVVVVVVVVVVVVVVVVVMVVLVVVVVV |
| 565 | E | 0.987 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 566 | R | 0.977 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 567 | Y | 1 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 568 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 569 | K | 0.622 | # KRKKERKRTRMKRRKRRRRKQKGKGKQQKKKKKKKGKRRQKKKRRKRRKRGKKRKQKKKKKKKKKKQKKRRKKKKRRRKRRR |
| | | | RRRRRRRRRRRKRRRRRKKRRKKRKQQQKKRKKKKRRRKKKRRRRKRKKGKKKKRKKRKKVRRKRRKQKQKK |
| 570 | D | 0.987 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDD |
| 571 | Q | 0.987 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 572 | Q | 0.977 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 573 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 574 | L | 0.96 | # LLLLLLLLLLLLLLLLLLLLLLLLLLLLLLLLLLLLLLLLLLLLLLLLLLLLLLLLLLMLLLLLLLLLLMLLLLLLLLLLLL |
| | | | LLLLLLLLLLLLLLLLLLLLLLLLLLLLLLLLLLLLLLLLLLLLLLLLLLLLLLLMLLLLLLLLLMLLLLLL |
| 575 | G | 0.974 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 576 | I | 0.956 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 577 | W | 0.974 | # WWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 578 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 579 | C | 1 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 580 | S | 0.974 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 581 | G | 0.987 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 582 | K | 0.939 | # KKKKKKKKKKKKKKKKKKKKKKKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 583 | L | 0.964 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 584 | I | 0.987 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 585 | C | 0987 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 586 | T | 0.942 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 587 | T | 1 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 588 | A | 0.632 | # AAAASTTTAAATNTAAATAAAAATATATANATAAAAAAAAAAAATAAATAAATTAAATAAANANTNANNNNTAAATSTATTT |
| | | | NNTNNATSNNNNTANSNATAATAATTTTTATAATTTTAATTTTTT-ATAAATAATTTTATATAAASTATTAA |
| 589 | V | 0.974 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVLVVVVVVVVVVVVVVVVVVVVVVVVVVVVVVVVVVVV-VVVVVVVVVVVVVVVVVVVVVVVVVV |
| 590 | P | 0.987 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPP-PPPPPPPPPPPPPPPPPPPPPPPPPP |
| 591 | W | 0.948 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWW-WWWWWWWWWWWWWWWWWWWWWWWWWW |
| 592 | N | 0.961 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNDNNNNNNNNNNNNNNNNNNNNNNNNNNNNNNNNNNN-NNNNNNNNNNNNNNNNNNNNNNNNNN |
| 593 | A | 0.635 | # DTAAAAATAAAASAATAATANADAAAATATAAAAAATTTTTTTAATAATAATAATAAATAATATTTVTTTTAATATATAAAS |
| | | | TTATVATATVAVTSTAITASATAAATTTAAADTTSAAAAAAAATA-TAAAAAAATTTTATAAATAVAAATVT |
| 594 | S | 0.964 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSS-SSSSSSSSSSSSSSSSSSTSSSSSSS |
| 595 | W | 0.977 | # WWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWW$WWW-WWWWWWWWWWWWWWWWWWWWWWWWWW |
| 596 | S | 0.961 | # SSSNSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSS-SSSSSSSSSSSSSSSSSSSSSSSSSS |
| 597 | N | 0.935 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | SNDNNNNSNNNNNNNNNNNNNNNNNNNNNNNNNNNNNNNNNNNNN-NNNNNNNNNNNNNNNNNNNNNNNNNN |
| 598 | K | 0.875 | # KKKKKKKKKKKKKKKKKKKKKKKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKK-KKKKKKKKKKKKKKKKKKKKKKKKKK |
| 599 | S | 0.843 | # SSSSSSSTSSSSSSSSSTSSPSSTSSSSSSSSSSSNSSSSSSTSNSSNNSSSSTSSSSSSSSSSSSSSSSSSSSSSSNSSSS |
| | | | SSSSSSSNSSSSSSSSTSSSTSSSSSSSSSSTSSSSSSTSSSSSS-SSSSSSSSSSSSSSSSSSSSTSSSSS |
| 600 | L | 0.63 | # VLYVLLLLLLLLLLLLMLLLLLLMLQLLLLRLYLLLYLLILLMQLQLLLVLELLMLLLFLVQLQMQHQLLELLLLFLLLVLL |
| | | | LLHFLQLLLLLMLLLLYLLLLLLQLLLLLLMLLLMLLLLLLLLLL-LLLLLLLLLYLLLRFLLLLVLLQLLL |
| 601 | D | 0.481 | # DNNDSDDEEDENSEHDDDSEDEKDNDSNDDDNSDDSNTTDDKQDSDDTSDNSDGENDDNEDDNTETDDNNDDDNNNNSNDND |
| | | | SNDNNDNTNNTDNDNTDDDGDDSANDDDDEDDDDTNNEDDDNDDN-DDDDDEGTDNENNDKDEDDDENDDDD |
| 602 | E | 0.365 | # YDDDDQKKKQEQKTEEMQQQQEYQQEQQQQEEDEEQQEEAQAEEQENEQYQDEEQEKKEEYDEDDEEDTDENTEEYYDEYYE |
| | | | DYYDEYEDDEEDDKYDEKTDMDDDAKKKQQKQQKEQMDQDKKKKQ-KKERTKQDTDKEQEEQERKDAEYTYK |
| 603 | I | 0.987 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIII-IIIIIIIIIIIIIIIIIIIIIIIIII |
| 604 | W | 0.968 | # WWWWWWWWWWWWWWWWWWWWWWWWWWWWWWWWWWWWWWWWWWWWWWWWWWWWWWWWWWWWWWWWWWWWWWWWWW$WWWWWWW |
| | | | WWWWWWWWWWWWWWWWWWWWWWWWWWWWWWWWWWWWWWWWW$WWW-WWWWWWWWWWWWWWWWWWWWWWWWWW |
| 605 | D | 0.614 | # RNDNDNNDNNGDDENNNNDNNNDDNKDNHDDDDNNDDDDHNDDNDNNDDENNDNDDNNDDENNDDDDDENDNNDNDDDDDDN |
| | | | DDGNNNNDDDDNDDDDNDNDDHDNDNNNNNNNDDDDNDNNDNNGD-NHDNNDDDNKNDDDNDGNQNNDNGNN |
| 606 | N | 0.949 | # NYNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNN |
| 607 | N | 0.915 | # MTMMMMMMMMMMMMMMMMMTMMMMMMMMMMMMMMMMMMMMMMMMMMMMMMMMMMMMMTMMMMMMMMMMMMMMMMIMMMMMMM |
| | | | MMMMMMMLMMMMMMMMMMMMMMMMTMMMMMMMMMMLMMMMMIMMMMMMMMMMMMMMMMMMMMMMMMMMMMMM |
| 608 | T | 1 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 609 | W | 0.987 | # WWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 610 | M | 0.926 | # MMMMMMMMMMMMMMMMMMMMMMMMMMMMMMMMMMMMMMMMMMMMMMMMMMMMMMMMMMMMMMMMMMMMMMMMMMMMMMMMMM |
| | | | MMMMMMMMMMMMMMMMMMMMMMMMMMMMLMMMMMMMMMMMMMMMMMMMMMMMMMMMMMMMMMMMMMMMMMMM |
| 611 | E | 0.719 | # QQQEQQEQEQQEEEEQEEQEEEQEQEEQQEQQEEEEEQQEEQQQEQEEQEQEQQEEEEEEEQQQQQEQQEEEQQEQQEEQQQ |
| | | | QQQQEQEQEEGEQEQQQEEKEQEQQEEEEEEDEEEEQQEQEKEEEQEEEEEEEEQQEKQQEEKEEEQEEQQE |
| 612 | W | 0.977 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 613 | E | 0.797 | # EEEEEEEEEEEEEEEEEEEDEEEEEDEEEEEEDEEEEEEEEKEEEEEEEEEEDEEEEEEEEDEDEDEDDDDEEEEDDDEDDD |
| | | | DDDDDDEEDEEDDEDDEEEEEEEEEEEDDEEEDEEEEDEEXEEEEEEEEEEDEEEDEEEEEEEEEDDEEEEE |
| 614 | R | 0.82 | # RKRRRRKTRRKRRRRRRRRRRRKRRRKRRRRRRRRRRKKRRRKRRRRRKKRRRKRRRRRRRRKRRRKRRRRRKRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 615 | E | 0.964 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 616 | I | 0.977 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 617 | D | 0.634 | # DDDDGNDNHDRDDDDDDDDNDDDSDNDDDDSDNDDDDDDDDDEDDDDDEDDDSDSEDDNDDDDDENDDNNNDDDDNDNDNND |
| | | | NNNNNNDENDDNNDNTDNSDENDDSDDDANDDDNGDDDDDDNDDDDDDDDNDNNDDNDESNNNDDDDNNNDD |
| 618 | N | 0.961 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNN |
| 619 | Y | 0.956 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 620 | T | 0.977 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 621 | S | 0.4 | # DSGSGSDNKRKGGDRGSSSSGSGRRNGSGGEHNNNDGSSNNGGDSGSSDNSNEGDGNNNSGNSRGSSRNNNGGNREEKSNTN |
| | | | SDNNSNSSDSKSENDEDDDSGDNGVGGGNSSSNTGSGNHSGSSSSSSSNSNGSSGGSHDSRKDSEDSSGGSG |
| 622 | L | 0.643 | # HILLLLLILLYLLLELLLLLLLTTLLLEILLLLEEELLLSLLLLLLLLVLYLLLLLLTLLLLLLLLILLLLLLTEYYFPYLL |
| | | | YYYYLLLLYLLLYLYLHLLYLLLLLLLLLLLLTLLIITLLLLLLLLLLLELLLLLLLILQLLLLLYLYLLLL |
| 623 | I | 0.974 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 624 | Y | 0.968 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 625 | T | 0.581 | # LSTNHTTTSTSRTSTTTTNSTTQTNNTYNNSTTATRNTTSTTTTTTTNTTATSTTNTTNTYTTTTTTTTTDNTDTTTTNTTS |
| | | | TTTTSSNTSTTTTTTTNNNTTTSTSTTIHSTNTTNSNTTSTSSTTTTTTTTTTTTNTSNTTTTTSDSTNNTT |
| 626 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 627 | I | 0.78 | # LIIILIILIILIIIIIIIIIIIIIIIILIIIILIIIIIILIIIIILLLILLIILILIIIILLILILLLLLLIIIILLLLLLL |
| | | | LLLLLLLLLLLLLLLLLIIIIIILILLLIIIILLIILLILIIIIIIIIIIIIIILIIIIIIIIILLILIIII |
| 628 | E | 0.923 | # EEQEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 629 | E | 0.617 | # NEIKEEQEEEEEEKEEEQEEEEEEQQEKEKEEEQQQKEEKEEENEEKEQNKEKEKTEEEEKEEEEETEEEKQEEEGDEEDDE |
| | | | DDDDEDEENEEENEDDKEEEEEDEEEEEEEEEEEIEEEEKEKEEEEEEEEEKEEEEEQEEEEEEQKEKEEEE |
| 630 | S | 0.882 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | AAAASSSSASSSASAASSSSSSSSSSSSSSSSSSASSSSSSSSSSSSSYSSSSSSSSSSSSSSSSSSSSSSS |
| 631 | Q | 0.964 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQPQQQQQ |
| 632 | N | 0.793 | # NTINNNNNNNNNNKNNNNNNNTYNNTINNNNNNNNNNNNNNYNNNNNNNNNNNTNNNNNNIININNNINNNNNNNNNNNNNN |
| | | | NNDNNNNNNNNNNNNNNNNNNNINNNNNNNNNLNNNNNNTINNNNDNNNNNNINNNNNINNNNNTINNNNNN |
| 633 | Q | 0.987 | # QQQQQQQQQQQQQQQQQQQQQQQQQQQQQQQQQQQQQQQQQQQQQQQQQQQQQQQQQQQQQQQQQQQQQQQQQQLQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 634 | Q | 1 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 635 | E | 0.942 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 636 | K | 0.942 | # KKKKKKKKKKKQKQKKKKKKKKKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 637 | N | 1 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNN |
| 638 | E | 0.974 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 639 | Q | 0.822 | # QQQQQQQKQQQQQQLQQQQQQLKLQQQKQQQQQLLQQQQQQKQQQQQQQQQKQQQQKQQQQQQQQQQQQQQQQQLQQQQQHK |
| | | | QQQQQHQQQQQQQQQQQQQLQQKQLQQQQQQQQQQQQQQQQQQQQQQLQQQQQQQQQQQQQQQQQKQQQQLL |
| 640 | E | 0.853 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEDDEEEDEEDEEEEEEEEEEEEEEEEEGEEAEEEEEEEEEEEEEEEEEEEEEEEEEDE |
| 641 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 642 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLSLLLLLLLLLL |
| 643 | E | 0.768 | # EAEEAEEEEVEKEEEEEEEEEKEEEEEEEEEEAEEEEGGEAEAEEEEEEEEEAEEEEEAGVEEEAEEEEEEEEEEEEEEEEE |
| | | | EEEEAEEEEEEEEEEEEAEEAEEEEAAAEEEEEEEEEEQEEKEEEEEEEEEEEGEEEAEEAEKEEEEEEAEK |
| 644 | L | 0.974 | # FLLLLLLLLLLLLWLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 645 | D | 0.943 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDD |
| 646 | K | 0.845 | # KKKKKKKQNKHTKKKKKKKKKKKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 647 | W | 0.968 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 648 | A | 0.873 | # AAAAAAAAAAAAAAAAEAAAAAAAAAAAATAASAAAAAAAAADAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 649 | S | 0.832 | # NSSNSNSNSSSSSSSSNSSSSSSSSSSSSSNGSSSNNSSSSSSSSSSSSSSNSSSNSNSSNSSNSSSSSSSNSSSSSSNSSS |
| | | | SSSSSSNNSNNSSNSSSSSSSSSSSSSSSSSSSSSSSSSSSSSSSSSSSSSSSSSSSSSSSSSSSSSSSSSS |
| 650 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 651 | W | 0.977 | # WWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 652 | N | 0.766 | # NNNNNNNNNNNNNNNNSSNNDNNNSNNNNSTSNSSNSNNTNNNNNNNSNNNSSDNNNNNNSNNNNNNNSNNSDNNNNSSNNN |
| | | | NNNTNNSNNNNNNNNNNNNSNNNNNNNNSNTNSTNNNNTNNSSNNNNNNNSNNNNNNNNNNSNNNNNSTNNN |
| 653 | W | 0.987 | # WWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 654 | F | 0.974 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 655 | D | 0.675 | # NEDDSDDTDDDSDSDDDDSNSDDDSDSDDDDSDDDSDNDDDDSDSSDSSDDSDSDNDDSTDDSDNDSDDDNSDSDDNDDDDD |
| | | | DDDDDDDDDDDDDDDDDDDDDDDDDDDDDDDDDDDDDDDDDDDDDDDDDDDDDDDDDDDDDDDDDDDDDDDD |
| 656 | I | 0.987 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 657 | T | 0.844 | # SSTTTTTSTTTTTTTTSSTTTTTSTTTTTTTTSTTSTTTTSSTTTTTTTTTSSTTTTTTTTSTSTTSSSTTTTTTTTTTTTT |
| | | | TTSTTTTTTTTTSTTTTSTTSTTSTTTTSTTTTTTTTTTTTTTTTTTTTTTTTTTTTTTTTTTTTTTTTTTT |
| 658 | N | 0.592 | # NNQQKKNNKSNKKNKNNNNKKNNQNNNNNQNNQQNNHHNNNKKNHNNSKKNNNNNNNKNNNQNNNNNNKKNKNKKKNQNKHS |
| | | | NNNHRENKHNQNHNSNKENNNNNNQQQNNNNHQNNQNKNKKKNNKKKNKKKKNNNNKNHNNQQQNNNNNNNK |
| 659 | W | 1 | # WWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 660 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 661 | W | 0.987 | # WWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 662 | Y | 1 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 663 | I | 0.987 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 664 | K | 0.865 | # KKKKKKKKKKKKKKKKKKKKKKKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 665 | I | 0.926 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 666 | F | 0.974 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 667 | I | 0.974 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 668 | M | 0.903 | # MMMMMMMMIMMMMMMMMMMMMMMMMMMMMMMIMMMMMMMIMMMMIMMMMMMMMMMMMMMMMIMIMMMMMIMMIMMMMMMMMM |
| | | | MMMMMMMMMMMMMMMMMMMMMMMMMMMMMMMMMMMMMMMMMMMMMMMMMMMMMMMMMMMMMMMMMMMMMMMM |
| 669 | I | 0.935 | # IIIIIIIIVVVIIIIIIIIIIIIIIIIIIIIIIIIIIIIIIIIIIIIIIIIIIIIIIIIIIIIIIIIIIIIIIIIIIIIIII |
| | | | MIIIIIIIIIIIIIIIIIIIIIIIIIIIIIIIIIIIIIIIIIIIIIIIIIIIIIIIIIIIIIIIIIIIIIII |
| 670 | V | 0.917 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 671 | G | 0.929 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 672 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 673 | L | 0.977 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 674 | I | 0.753 | # VIVIVVVIIVVIIVIVVVIVVVVIIVVVVVVIVVVVIVIIVVVIVIVVIIVIVIVIIIIVVIVVVIVIVVVVIVVVIIIIII |
| | | | IIIIIIIIIIIIIIIIIIIIIIIIIIIIIIIIIIIIIIIIIIIIIIIIIIIIIIIIIIIIIIIIIIIIIIII |
| 675 | G | 0.987 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 676 | L | 1 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 677 | R | 0.948 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 678 | I | 0.974 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 679 | V | 0.873 | # VVIVVVVVVIVIVVVVVVVVVVVVVVIVVIVVVVVVVVVVVVVVVVVVVVVVVVVVVIVVIIVIVIVIIIIVVVIVVVVVVI |
| | | | VVVVVVVVVVVVVVVVVVVVVVVVVVVVVVVVVVVVVVVVVVVVVVVVVVVVVVVVVVVVVVVVVVVVVVVV |
| 680 | F | 0.949 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 681 | A | 0.608 | # TTTTATITATAAAVAAAATATATTATTATMIAAAATTTTVATTTTAATTAAAAATTTATTSAAATSAASSFAVAAATAAAAA |
| | | | AAAAAAAAAAAAAAAAAAAAAAAAAAAAAAAAAAAAAAAAAAAAAAAAAAAAAAAAAAAAAAAAAAAAAAAA |
| 682 | V | 0.953 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 683 | L | 0.878 | # LLLLLLLFILLLLLLFLLLLLLLLLLLLLFLLLLLLILPLLLFLLLLLLLLLLFLLLILLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 684 | S | 0.933 | # STSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 685 | I | 0.867 | # IIIVLIVIIIIIIIIIIIIIIIIIIIIIIVVIIIIIILLLIIIIIIIIIIVIIIIILTIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 686 | V | 0.932 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 687 | N | 0.882 | # NNNNKNNNTNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNN |
| 688 | R | 0.955 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 689 | V | 0.987 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 690 | R | 0.987 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 691 | Q | 0.933 | # KQQQEQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 692 | G | 0.974 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 693 | Y | 1 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 694 | S | 0.964 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSLSSSSSSLSSSSSSSSSSSSSSSSSSSSSSSSSSSSSSSSSSSSSSSSSSSSSSSSSSSSSSSSSSSS |
| 695 | P | 0.987 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 696 | L | 0.933 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 697 | S | 0.977 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 698 | F | 0.854 | # FLFFLFFFFFFFFFLLFFFFFFLLFFFFFFLFFLLFFLLLFFFFFFLFFFFFFFFFFLFFLFFFFFFFFFFFLFLLFFFFFF |
| | | | FLFFFLFFLFFFFFFFFFFFFFFFFFFFFFFFFFFFFFFFFFFFFFFFFFFFFFFFFFFFFFFFFFFFFFFF |
| 699 | Q | 0.974 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 700 | T | 0.977 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 701 | R | 0.678 | # RLRRRHRHHLLLRHRRRLRNRRRHRHRLRRHRHLLRRRRHHRHRRHRHRLHRRHRHRHRRLHHHRHHHHHHHHRRHHLHHHL |
| | | | HHHHRHHHHHHRHHHHHRLRHRRHRHHHHHHRLRRHHRLRRRRRRRRRRRRRRRRRRRRRRRRRRRRRRRRR |
| 702 | L | 0.785 | # FLLLPLFLLLLLLLFLLLLLLLLLFLLLLLLLLLLLLFFLLPLLHLFLLLLLLLLPFLLLLLLLLLLLFLFLLLFLLLLLLL |
| | | | LLLLLLLLLLLLLLLLLLLLLLLLLLLLLLLLLLLLLLLLLLLLLLLLLLLLLLLLLLLLLLLLLLLLLLLL |
| 703 | P | 0.987 | # PPPQPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 704 | A | 0.626 | # TAAAAAATATTATAAGAAATAATVAATAAAATAAAATTTAVATAAAAAAAATTAAAVTAAATSASASASSSSAPAAAATAAA |
| | | | VAAATAAAAAATAAAVTTAAAVGATTTTAIIAVAATAVNVAAASAAAATAAAAAAAAATAAAVAVAAGAAAA |
| 705 | P | 0.652 | # SQQPPPPPPPPPPPQPPTRPQRQPPQQPQQPQRPPRPPPPPAQPPPPPQPAPPPRQPP$PQPPPRPPPPPPQRQQRRPRRRP |
| | | | RRRRPPRRRRRPRRRRPQPPPPRPPPPPPPPPPPQPPPPPQPPQRPPPPPRPQPPQPQPPPPPQPSPPPSQQ |
| 706 | R | 0.964 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 707 | G | 0.892 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 708 | P | 0.974 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 709 | D | 0.925 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDD |
| 710 | R | 0.987 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 711 | P | 1 | # PPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 712 | E | 0.797 | # EGEEEEEEEEEEEEEEEEEEEEEGDEEEEEEEEEEEEEEEEGEEEEDEEEEEEDEGEEEEGGGGEGEGGGGEDDEEEGEEEE |
| | | | EEEEEEEEEEEEEEEEEEEEEEEEEEEEEEEEEEEEEEEEEEEEEEEEEEEEEEEEEEEEEEEEEEEEEEEE |
| 713 | G | 0.953 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 714 | I | 0.869 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | TITTIIIITIIITIITIIIIIIIIIIIIIIIIIIIIIIIIIIIIIIIIIIIIIIIIIIIIIIIIIIIIIIII |
| 715 | E | 0.918 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 716 | E | 0.847 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | GEGGGEEGGEEGGEEEEEEEEEEEEDDDGEEEEEEEGEEEEEEEEEEEEEEEEEEEEEEEEEEEEEEEEEEE |
| 717 | E | 0.911 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEGEEEEEEEEEEEEEEEEEEEEEEEEEEEEEEEEEEEEEEEEEEEEEEEEEEEEEEEEEEEEEEEEEE |
| 718 | G | 0.977 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 719 | G | 0.987 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 720 | E | 0.859 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 721 | R | 0.756 | # RSRRQRRRRRRRRRRRRRRRRRRRRRRRRRRRRRRRRRRRRRRRRRRRRRRRRRRRRRRRRRRRRRRRRRRRRRRRRRRRRR |
| | | | RRRKRRRRKRRRRRRRRRRRQRQRRRRRRRRRRRRRRRRRRRRRRRRRRRRRRRRRRRRRRRRRRRRRRRRR |
| 722 | D | 0.933 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDD |
| 723 | R | 0.865 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 724 | D | 0.855 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDD |
| 725 | R | 0.766 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRTRRRRRRTRGRRRTRRRRRRRRRRRRRRRRRRRGTRRRRRRRRRRRRRRRRRRRRRRRRRRRRRRRRR |
| 726 | S | 0.987 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 727 | G | 0.447 | # GERRVGETTSTGRGESNVGIGVVGSNGGGGGGGGNDEVSGGGTVGGVSSGEGIGIGGGGVGGGGGSGSGTGVKVIVVIVVVV |
| | | | VVVVVLVNVVIVVVVIEIGGVGSGIGGGVIVGISNVGVTGGGGGGGGGGGGGGGGGGGGGGGGGGGGGGGRG |
| 728 | R | 0.593 | # RERYRRRRRRRRQPIRPRRQRQRQRRPGRHRRPHYLRRRRRGRQFPRQPRPPIRSPRRPQRRRAHRRRRRRRRRRRRRRRRR |
| | | | RRRRRRRRRRRRRRRRRRRRRRRPRRQQQRRRRRPRRGRRRPQRRRRRRLQRRRRRRPRLRQLRRPLRRPQP |
| 729 | L | 0.92 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLSLLLLLLLLLLLLLLLLLLLLLLLLLLLLLLLLLLLLLLLLLLLLLLLLLLLLLLL |
| 730 | V | 0.946 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 731 | D | 0.494 | # TGTDTDHHHHTHDTDKSDNNDNNNDTADDDDDNNTDDDTDENDDDTDDQDDNNTTDDDNEHDNRHDNDDNDDHHSHHHHHHH |
| | | | HHHHNTHHDNHHHDNDDDDDNDDDDDNDDDHHHNDHHHHHNNDDDGNDDNNDNNDNNDNDDHDNHNDTDDDN |
| 732 | G | 0.974 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 733 | F | 0.821 | # LFFFFFFFFFFFLLFFFFFSFFFFFFFFFLFFFLLFFFFFFFFLFFFFLCFFFFFFFFFLLFFLFFFFFFFFFFFFFFFFFF |
| | | | FFFFSFFFFFSFFFFFFFFFFFFFFFFFFFFFFFFFFFFFFFFFFFFFFFFFFFFFFFFFFFFFFFFFFFFF |
| 734 | L | 0.927 | # FLSLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 735 | A | 0.765 | # AAATAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAPAAAAAAAAAAAAAAAAAAAAAAAAAAAAAAAAAAAAAAAAAAAAAAAAAAAAAAAAAAAAAAAA |
| 736 | L | 0.802 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 737 | I | 0.748 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 738 | W | 0.964 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 739 | D | 0.664 | # DVDGDVVDDVDGDVEVDDVDVDVDEVVDVIVDVDDVDVVDDVVVVVDVVDVVVDVDDDVVVVDVIVDVVVVDDDEDDDDDDD |
| | | | DEDDDDDDDEDDDDEDVDDVDDVVDVVVEDDVDDDDDEDVVIIVVVDDVVDVVDVVVDVVDVDDVVVVVVVV |
| 740 | D | 0.974 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDD |
| 741 | L | 0.974 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 742 | R | 0.924 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 743 | S | 0.902 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 744 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 745 | C | 0.848 | # CCCCCCCCCCCCCCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCC |
| 746 | L | 0.933 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 747 | F | 0.974 | # FFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFF |
| 748 | S | 0.876 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSLSSLSIS |
| 749 | Y | 0.974 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYCY |
| 750 | H | 0.939 | # HHHHHHHHHHHHHHHHHHHHHHHHHHHHHHHHHHHHHHHHHHHHHHHHHHHHHHHHHHHHHHHHHHHHHHHHHHHHHHHHHH |
| | | | HHHHHHHHHHHHHHHHHHHHHHHHHHHHHHHHHHHHHHHHHHHHHHHHHHHHHHHHHHHHHHHHHHHHHHHH |
| 751 | R | 0.849 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 752 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 753 | R | 0.961 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 754 | D | 0.977 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDD |
| 755 | L | 0.968 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 756 | L | 0.948 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 757 | L | 0.96 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 758 | I | 0.912 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 759 | V | 0.81 | # AVAAAVVVVVVVVVVVVVVVVVVVVVVVVVVVVVVVVVVVVVVVVVVVVVVVVVVVVVVVVVVVVVVVVVVVVVVVVVVVVV |
| | | | VVVVAVVVVVAAVVVVVAVAVVVV-VVVVVALVVKVVAVAVVVVVVAAVVVVVVVVVVVVVVVVVVVVVVVV |
| 760 | T | 0.707 | # ATAAATTTTTTATAATAATTATTAAATATTTATTTAATTTATTTATAKTTTTTATTATTTTATATAAAAAVTTTTAATAATA |
| | | | TATTAAAATTAATTATTATAAATT-TTTTTATTARKVAAAAAATTTAAATTTTATTTTTATATAATTKTTTT |
| 761 | R | 0.92 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRGRRRRRRRRRRRRRRRRRRRR-RRRRRRRRRRRRRRRRRRRRRRRRRRRRRRRRRRRRRRRRRRRRRRR |
| 762 | I | 0.797 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | ITIIIIIIIIIIIIIIIIIIIIII-TTTIIIITIIIITIIIIIIIIIIIIIIIIIIIIIIIIIIIIIIIIII |
| 763 | V | 0.956 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 764 | E | 0.964 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 765 | L | 0.81 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | TILLLITILTTLLTITLLLLLLLLLLLLLLLLLLLLLLLLLLLLLLLLLLLLLLLLLLLLLLLLLLLLLLLL |
| 766 | L | 0.974 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 767 | G | 0.974 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 768 | R | 0.848 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RQRRHRRRRRRHRRQRRRRRRRRRRRRRRRRRRRRRRRRRRRRRRRRRRRRRRRRRRRRRRRRRRRRRRRRR |
| 769 | R | 0.927 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | GRRRRRRRRRRRRRRRRRRRRRRRRRRRRRRRRRRRRRRRRRRRRRRRRRRRRRRRRRRRRRRRRRRRRRRR |
| 770 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 771 | W | 0.968 | # WWWRWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 772 | E | 0.93 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 773 | A | 0.559 | # TIIVAVAAAAAIIIVIVVAAAAAVGLTIIIAVAAAGAAAVAIVVAIIAAAIVAIAAAAVALIAIAVVIVIIIVVAALAIAVA |
| | | | IAVVIAIIATAIIIALAAVVVVVIAIIIAAVAAAPAAAVVAAAAVVIAVVAIAVAVAVAIAALAIAIIALIV |
| 774 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 775 | K | 0.948 | # KKKKKKKKKKKKKKKKKKKKKKKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 776 | Y | 1 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 777 | W | 0.822 | # LWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWEWWWWWWGWWWWLWWCWWWWWWWWWWWWWWWWWWWWWWWWWWWWWWWWWWWWWWWWWWWWWWWWCLWW |
| 778 | W | 0.871 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWGGWWWWWWWWGWWGWWWWWWWWGWW$WWWWWWWWWWWWWWWWWWWWWWWWWWWWWWWWWWWWWWWWWWWW |
| 779 | N | 0.933 | # SNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNN |
| 780 | L | 0.964 | # LLLILLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 781 | L | 0.987 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 782 | Q | 0.882 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QLQQQQQQQQQQQQLQQQQQQQQQQQQQQQQQQQQQQQQQQQQQQQQQQQQQQQQQQQQQQQQQQQQQQQLQ |
| 783 | Y | 1 | # YYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYY |
| 784 | W | 0.955 | # WWW$WWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWW |
| 785 | S | 0.777 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSGSSSSSSSSSIIRSSSSGISSSSSSSSSSSSSSSSSSSSSSSSSSSSSSSSSSSSSSSSSSSSSSS |
| 786 | Q | 0.929 | # QQLQQQQQQQQQQQQQQQQQQQQQQQQQQQQQQQQQQQQQQQQQQQQQQQQQQQQQQQQQQQQQQQQQQQQQQQQQQQQQQQ |
| | | | QLQQQQQQQQQQQQQQQQQQQQQQQQQQQQQQQQQQQQQQQQQQQQQQQQQQQQQQQQQQQQQQQQQQQQQQ |
| 787 | E | 0.987 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 788 | L | 0.968 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 789 | K | 0.846 | # KKKKQKKQQKQKKKKKKKKKKKKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKKK |
| 790 | N | 0.785 | # NSNNNNNKKKKNNSNNNNNNNNKNNDNKNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNN |
| 791 | S | 1 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 792 | A | 0.974 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 793 | V | 0.895 | # VVVVIVVVVIVVVVIVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 794 | S | 0.877 | # SSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSS |
| 795 | L | 0.974 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 796 | L | 0.778 | # LFFFLFLFLYFLLFLFLFLLLLFFLLLLLFLLLLLLFLLLLLLLFFLFFIFLFLFLFLFFLLFLLLLLLLLLLLLLLLLLLL |
| | | | LVLLLLLLLLLLLLLLLLLLLLLLLLLLLLLLLLLLLLLLLLLLLLLLLLLLLLLLLLLLLLLLLLLLLLLL |
| 797 | N | 0.877 | # NNNNNNNNNNNNNNNNNNNNNNNNNNNNNNNNNNNNNNNNNNNNNNNNNNNNNNNNNNNNNNNNNNNNNNNNNNNNNNNNNN |
| | | | DNDDNDNNNDDDDNNNNNNNNNNNNNNNNNNNNNNNNNNNNNNNNNNNNNNNNNNNNNNNNNNNNNNNNNNN |
| 798 | A | 0.779 | # AATAAAVAAAATAAAAATAAAAATTAAAAAAAAAAAATTTAAAAATTAAAATAAATTTAAAAAAAAAAAAAAAAAAAAAAAA |
| | | | ATVASAATTAASAATAAAATAAAAAAAAAAAAAAAAAAAAAAAAAAAAAAAAAAAAAAAAAAAAAAAAAAAA |
| 799 | T | 0.79 | # TVIITITTTTTTTTTTTTTTTTTTTTTTTTTTTTTTTTTTTTTTTTTTTTTTTTTTTTTTTTTTTTTTTTTTTTTTTTTTTT |
| | | | IIITTTTITTTITIIITTITTTTTTIIIRTTTTTTTTTTTTTTTTTTTTTTTTTTTTTTTTTTTTTTTTTTT |
| 800 | A | 0.977 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 801 | I | 0.907 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 802 | A | 0.91 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 803 | V | 0.938 | # VVVVVVVVVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVV |
| 804 | A | 0.926 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 805 | E | 0.974 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 806 | G | 0.977 | # GGGRGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 807 | T | 0.956 | # TTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTT |
| 808 | D | 1 | # DDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDD |
| 809 | R | 0.954 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 810 | V | 0.773 | # IIVIVVVVVVVVVVVVVVVVVVVVVVVVVVVVVVVVVVVVVVVVVVVVVVVVVVVVVVVVVVVVVVVVVVVVVVVVVVVVVV |
| | | | VIVVVIIIIVIVIIIIVVVVVVVVVVVVVVVVVVVVVVVVVVVVVVVVVVVVVVVVVVVVVVVVVVVVVVVV |
| 811 | I | 0.896 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 812 | E | 0.943 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 813 | V | 0.671 | # VIGIVVVAVVVVIIVVVVVVAVVVVIVVIVVIVVVIVVVVVIVIVVVVIVVVIVIVVVIIVLALIIVLIIIVVVVIVAVVVV |
| | | | IIVVVIVIAVVVIGIVVVLIVIVGAVVVVVVVVVVIVVVVVVVVVVVVVVVVVVVVVVVVVVVVVVVVVVVV |
| 814 | V | 0.595 | # GAVVVLLLLLLLVVFVLVVLVALIVVSAVLIIVLIIVILLLVFVVIVVILVLLVVVVVVLLLLLLVLVYLLLLLVVVVVVVV |
| | | | VAVVVIVAVVVVVVAVVVLIVLVLVLLLLLLVLVVLVVVAAVLAVVLLLVVVVVVVVVVVVVVVVVVVVVVV |
| 815 | Q | 0.866 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QRRQQRQQRRRQQQRLQRQQQQQQQQQQQQQQQQQQQQQQQQQQQQQQQQQQQQQQQQQQQQQQQQQQQQQQ |
| 816 | R | 0.923 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 817 | A | 0.585 | # LILIAAITAAVIAAAAAAAAIIAAVAVAVTTAIAAIAVVIIAAIATAAIAIIATAIAATAIAIATAFAAAAAIIAIIIYLIL |
| | | | VIIVAAIIIAAAIIIIVAIAIIAAAAAALAAAIIIIIIAIAITTAAAIAATIAAAAAAAAAAAAAAAAAAAA |
| 818 | F | 0.506 | # GGCVYYYGYGYFYGWCCCCCYCCFFGCFFYFCCFFFFFCYCGCWFFCFFFFCYWCYYFGYCYGCLYYYYGCCYFFCFFFFFC |
| | | | FFFFFFFFFFFFFFFFFFFFFFFFFFFFFFFFFFFFFFFFFFFFFFFFFFFFFFFFFFFFFFFFFFFFFFFF |
| 819 | R | 0.974 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 820 | A | 0.935 | # AAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 821 | I | 0.72 | # IIIIIIIIIVIVIIIIIIIIIFIIIVITIIIFIIIVFIIFIF-IIIVIIFFIIIIIIIIIIIIIIIVIIIIVIIIFIIIVFI |
| | | | FFFFVIIIVVVIFIFIIFIIIFIIIIIIIIIIIIIIIIIIIIIIIIIIIIIIIIIIIIIIIIIIIIIIIIII |
| 822 | L | 0.783 | # LLLLLLLLLLLLLLLLLLLLLLLLLLLLLLLLLLLLLLLLLL-LLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 823 | H | 0.875 | # HNHHHHHHHHHHHHHHHHHHHHHHHHHHHHHHHHHHHHHHHH-HHHHHHHHHHHHHHHHHHHHHHHHHHHHHHHHHHHHHHH |
| | | | HHHHHHHHHHHHHHHHHHHHHHHHHHHHHHHHHHHHHHHHHHHHHHHHHHHHHHHHHHHHHHHHHHHHHHHH |
| 824 | I | 0.935 | # IIIIIIIIIIIIIIIIIIIIIIIIIIIIIIIIIIIIIIIIII-IIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 825 | P | 0.974 | # PPPPPPPPPPPPPPPPPPPPPPPPPPPPPPPPPPPPPPPPPP-PPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPP |
| 826 | R | 0.658 | # RTRTRRTRRRRRTTTVRRRRRRRRVVTRTTRRRRVTRRRRRV-VRRVRTRRRTRTRRTVTTRTRRRRRTVRRRTTTTRRTTT |
| | | | RTTRRTRVRVVRRVTTTRRRTRRRRRRRRRRRRRRRRRRRRRRRRRRRRRRRRRRRRRRRRRRRRRRRRRRR |
| 827 | R | 0.952 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RGRRRRRRRRRRRRRRRRRRRRRRRRRRRRRRRRRRRRRRRRRRRRRRRRRRRRRRRRRRRRRRRRRRRRRR |
| 828 | I | 0.908 | # IIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIII |
| 829 | R | 0.987 | # RRRRRRRRRRRRRRRRRRRRRRRRRRRRRRRRRRRRRRRRRRERRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 830 | Q | 0.987 | # QQQQQQQQQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQQQQ |
| 831 | G | 1 | # GGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGG |
| 832 | L | 0.836 | # LLLLLLLLLLLLLLLLLLLLLLLLLLLLLLLLLLLLLLLLLLLLLLLLLLLLLLLLLLLLLLLLLLLLLLLLLLLLLLLLLL |
| | | | FLLLFLLLFLLLLLLLLLLLLLLLLLLLLLLLLLLLLLLLLLLLLLLLLLLLLLLLLLLLLLLLLLLLLLLL |
| 833 | E | 0.974 | # EEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEE |
| 834 | R | 0.929 | # RRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRR |
| 835 | A | 0.794 | # AAAALAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAA |
| 836 | L | 0.956 | # LLLLLLLLLMLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 837 | L | 0.828 | # VLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLL |
| 838 | | | # $$$$$$$$$$$$$$$$$$$$$$$$$$$$$$$$$$$$$$$$$$$$$$$$$$$$$$$$$$$$$$$$$$$$$$$$$$$$$$$-$$ |
| | | | $$-$$$$$$$$$$$$$$$$$$$$$$---$$$$$$$$$$$$$$$$$$$$$$$$$$$$$$$$$$$$$$$$$$$$ |

SAMPLE #: 138

| | | | |
|---|---|---|---|
| | Vpr | | |
| AA# | Cons. | entropy | Raw Data |

TABLE 8A+TL,56

| | | | |
|---|---|---|---|
| 1 | M | 0.966 | # MMMMMMMMMMMMMMMMMMMMMMIMMMMMMMMMMMMMMMMMMMMMMMMMMMMMMMMMMMMMMMMMMMMMMM |
| | | | MMMMMMMMIMMMMMMMMMMMMMMMMMMMMMMMMMMMMMMMMMMMMMMMIMMMMMMMMMMMMMMMMMMMM |
| 2 | E | 0.961 | # EEEEEEEEEEEEEEEEEEEEEEEEEEEEEEEEEEEEEEEEEEEEEEEEEEEEEEEEEEEEEEEEEEEEEE |
| | | | EEKEEEEEKEEEEEEEEEEEEEEEEEEEEEEEEEEEEEEEDEEEEEEEEEEEEEEEEEEEEEEEEEEEE |
| 3 | Q | 0.961 | # QQRQQQQQQQQQQQQQQQQQQQQQQQQQHQQQQQQQQQQQQRQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQ |
| 4 | A | 0.937 | # AAAAAAAAAADADAAAAAAAAAAAAAAAAAAAAAAAAAAAAAAAAAAAAATAAAAAAAAAAAAAAAAAAA |
| | | | AAAAAAAAAAAAAAAAAAAAAAVAAAAAAAAAAAAAAAAAAAAAAAAAAAAAAAAAAAAAAAAAAAVAA |
| 5 | P | 0.986 | # PPPPPPPPPPPPPPPPPPPPPPPPPPPPPPPPPPPPPPPPPPPPPPPPPPPPPPPPPPPPPPPPPPPPSP |
| | | | PPPPPPPPPPPPPPPPPPPPPPPPPPPPPPPPPPPPPPPPPPPPPPPPPPPPPPPPPPPPPPPPPPPPP |
| 6 | E | 0.958 | # EEEEEEEEEEEEEEEEEEEEEEEEEEEEEEEEEEEEEEEEEEEEEEEEEEEEEEEEEEEEEEEEEEEEEE |
| | | | EEEEEEEEKEEEEXEEEEEEEEEEEEEEEEEEEEEEEEEEEEEEEEEEEEEEEEEEEEEEEEEEEEQEE |
| 7 | D | 0.88 | # DDDNDDDDDDDDDDDDDDDDDDDDDDDDDDDDDDDDDDDDDDDDDDDDDDDDDDDDDDDDDDDDDDDDDD |
| | | | DNNDDDDDNDDDDDDDDDDDDXDNDDDDDDNDDNXDDDXDDVDDDDDDDDDDDDDDDDDDDDDDDDDDN |
| 8 | Q | 0.986 | # QQQQQQQQQQQQQQQQQQQQQQQQQQQQHQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQ |
| 9 | G | 0.966 | # GGGGGRGGGGGGGGGGGGGGGGRGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGRGGGGGGGGGGGGGGGGGGGG |
| 10 | P | 0.986 | # PPPPPPPPPPPPSPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPP |
| 11 | Q | 0.938 | # QQQQQQQQQQQQQQQQQQQQQQQQQQQQQQQQQQPQQQQQQQQQQQQQQQQQQQQQQQQQQQQQQQSQQQ |
| | | | QQQQQQQQQQQQQQQQQQQPQQQQQQQEQQQQQQQQQQQQQQQPQQQQQQQQQQQQQQQQQQQQQQQQQ |
| 12 | R | 0.952 | # RRRRRRRRRRRRRRRRRRRRRRRRRRRRRRRRR-RRRRRRRRRRRRRRRRRRRRRRRRRRRRRRRRRRRR |
| | | | RRRRRRRRKRRRRRRRRRRRRRRRRRRRRRRRRRRRRRRRRRRRRRRRKKRRRRRRRRRRRRRRRRRRR |
| 13 | E | 0.919 | # EEEEEEEEEIEEEEEEEEEEEEEEEEEQEEEEE-EEEEEEEEEEEEEEEEQEEEEEEEEEEEEEEEEEEE |
| | | | EEEEEEVEKEEEEEEEEEEEEEEEEEEEEEEEEEEEEEEEEEEEEEEEEEEEEEEEEEEEEEEEEEEEE |
| 14 | P | 0.986 | # PPPPPPPPPPPPPPPPPPPPPPPPPPPPPPPPP-PPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPP |
| 15 | Y | 0.848 | # YYYYYYYYYCYYYYYYYYHYYYYYYYYYYHYYY-YYYYYHYYHYYYYYYYYYYYYYYYYYYYYYYYYYHF |
| | | | YYYYYYYYYYYYYYYVYYYYYYYYYYYYYYYYYYYNYYYYYYYYYYYYSYNHYYYYYYYYYHYYYYYYH |
| 16 | N | 0.878 | # NNNNNNNNNNNNNNNNNNNNNNNNNNNNHNNNNNNNNNNNNINNNNSQNNNNNNNNNRPNNNNNNNNNNN |
| | | | NNANNNNNNNNNNXNNNNNNNNNNNNNNNNNNNNNNNNNNNNNNNNNNNNNNNNNNNNNNNNNHNNNNN |
| 17 | E | 0.851 | # EEEEEEAEEGEAEEEEEEEEEEEEEEEEVEEEEEAEEAEEEEEEEEEQEEEEEEEEEAEEEEEEEEEEEG |
| | | | EEEEEEEEKEEEEEEEEEEEEEEEEEEEEEEEEEEEEEEEEEEAEEEEEEEEEEEEQEEEEEEEDEEEE |
| 18 | W | 0.986 | # WWWWWWWWWWWWWWWWWWWWWW$WWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWW |
| 19 | T | 0.843 | # TTTATTTTTTTTTTTTTTTRATTAATTATTTTTAATTTTTTTTTAAAAATTTTTTTTTTTTTTTTTTTTT |
| | | | TTTTTTTTTTTTTTTTTTTTTTTTTTAAATAAAATTTTAATTTTTTTTTTTTTTTTATTTTTTTTTTTT |
| 20 | L | 1 | # LLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLL |
| 21 | E | 1 | # EEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEE |
| 22 | L | 0.986 | # LLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLILLLLLLLLLLLLLLLLLLLLLLLLLLLLLLLLLLLLLLLLL |
| 23 | L | 0.961 | # LLLLLLVLLLLLLLLLLLLLLLLLLLLLLLLLLLLLLLLLLLLLLLLLLLLLLLLLLLLLLLLLLLLLLL |
| | | | LLLLLLLLLSLLLLLLLLLLLLLLLLLLLLLLLLLLLLLLLLLLLLSLLLLLLLLLLLLLLLLLLLLLL |
| 24 | E | 0.947 | # EEEEEEEEEEEEEEEEEEEEEEEEEEEEEEEEEEEEEEEEEEEEEEEEEEEEEEEEEEEEEEEEEEEEEE |
| | | | EEKEEEEEKEEEEEEEEEEEEEEEEEEEEEEEGEEEEEEEEEEEEEEEEEEEEEEEEEREEEEEEEEEE |
| 25 | E | 0.903 | # EEEEEEEEEEEEEEEEEDEEDEEDDEEEEEEEEEEEEEEEEDEEEXEEEEEEEEEEEEEEEEDEEEEEEE |
| | | | EEEEEEEEKEEEEEEEEEEEEEEEEEEEEEEEEEEEEEEEEEEEEEEEEEEEEEEEEEKEEEEEEEEEE |
| 26 | L | 1 | # LLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLL |
| 27 | K | 0.986 | # KKKKKKKKKKKKKRKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKK |
| 28 | N | 0.644 | # NSSSNSSSNSSSSSNNSTSNTSNTTSSNNSRNNNNNSSNNSNSNSNNNNSSSNSSNSSSSSSNSSNNRSN |
| | | | RSNNSNSSNSSSNNNNNNNNNSTRNNNNNHNNNNSNXNNNNNNRNNNNNNNTSSSRNSSSNRNRNSNNS |
| 29 | E | 0.972 | # EEEEEEEEEEEEEEEEEEEEEEEEEEEEDEEEEEEEEEEEEEEEEEEEEEEEEEEEEEEEEEEEEEEEEE |
| | | | EEEEEEEEKEEEEEEEEEEEEEEEEEEEEEEEEEEEEEEEEEEEEEEEEEEEEEEEEEEEEEEEEEEEE |
| 30 | A | 0.972 | # AAAAAAAAAAAAAAAAAAAAAAAAAAAAAAAAAAAAAAAAAAAAAAAAAAAAAAAAAAAAAAPAAAAAAA |
| | | | AAAAAAAAAAAAAAAAAAAAAAAAAAAAAAAAAAAAAAAAAAAAAAAAAASAAAAAAAAAAAAAAAAAA |
| 31 | V | 0.93 | # VVVVVVVLVVVVVVVVVVVVVIVVDVVVVVVVVVVVVVVVVVVVVVVVVVVVVVVVVVVVVVVVVVVVVV |
| | | | VVVVVVVVVVVVVVVVVVVVVVVVVAVVVVVVVVVVVXVVVVVVVVVVVVVVVVVVVVVVVVVVVVVVV |
| 32 | R | 0.921 | # RRRRRRRRRRRRRRRRKWRKRRRKRRRRRRRRRRRRRRRRRRRRRRRRRRRRRRRRRRRRRRRRRRRRRR |
| | | | RRRRRRRRKRRRRRRRRRRKRRRRRRRRRRRRRXRRRRRRRRRRRRRRRRRRRRRRRRRRRRRRRRRRR |
| 33 | H | 1 | # HHHHHHHHHHHHHHHHHHHHHHHHHHHHHHHHHHHHHHHHHHHHNHHHHHHNHHHHHHHHHHHHHHHHHH |
| | | | HHHHHHHHHHHHHHHHHHHHHHHHHHHHHHHHHHHHHHHHHHHHHHHHHHHHHHHHHHHHHHHHHHHHH |
| 34 | F | 0.986 | # FFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFLFFFFFFFFFFFFFFFFFFFFF |
| 35 | P | 0.986 | # PPPPPPPPPPPPPPPPPPPPPPPPPPPPPPPPSPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPP |
| 36 | R | 0.927 | # RRRRRRRGGRRRRRRRRRRRRRKRRRRRRRRRGRRRRRRRRRRRRRRRRRRSRRRRRRRRRRRRRRRRRR |
| | | | RRRRRRRRRRRRRRRRRRRRRRRRRRRRRRRRRRRRRRRRRRRRRRRRRKRRRRRRRRRRRRRRRRRRR |
| 37 | I | 0.414 | # IITVPRVPPPTITVPPIIILIPMIIIIVTTIMPEIIIIIIIVIIIIIMIIPIVVVAVVVVVVVVVTMIIV |
| | | | PPVPVMMVTPGIRILIIIPNVIVPIIVMITXTAVIVIIVITTIPIMMMLILPILLPIIIPIPILIVPIV |
| 38 | W | 0.966 | # WWWWWWWWWWWWWWWWWWWWWW$WWWWWWWWWWWWWWWWWWWWWWWWWWWWWWWWWWWWWWWWWWWWWWW |
| | | | WWWWWWWW$WWWWWWWWWWWWWWWWWWWWWWWWWWWWWWWWWWWWWWWW$WWWWWWWWWWWWWWWWWWW |
| 39 | L | 0.972 | # LLLLLLLLLLLLLLLLLLLLLLLLLLLLLLLLLLLLLLLLLLLLLLLLLLLLLLLLLLIFLLLLLLLLLL |
| | | | LLLLLLLLLLLLLLLLLLLLLLLLLLLLLLLLLLLLLLLLLLLLLLLLLLLLLLLLLLLLLLLLLLLLL |
| 40 | H | 0.944 | # HHHHHHHHHHHHHHHHHHHHHHHHHHHHHQMHMHHHHHHHHHHMHHHHHHLHHMHHHHHHHHHHHHHHHH |
| | | | YHHHHHHHHHHHHHHHHHHHHHHHHHHHHHHHHHHHHHHHHHHHHHHHHHHHHHHHHHHHHHHHHHHHR |
| 41 | S | 0.664 | # GSNGSGDGGNSGDGSSGSGSSGSSSGGGGGGSGSGSSSSGSNGGSGGGGSAGGGNGGGGGGNGGGSGNGG |
| | | | SSGSSSGSSSNSGGDSSSSGGSNGGGSGSSGGSGSSGSGSGGSSGGGGSSSSNSSSGNGGSGGSSSGSG |

TABLE 8B+TL,56

| | | | |
|---|---|---|---|
| 42 | L | 1 | # LLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLL |
| 43 | G | 0.933 | # GGGGGGGGGGGGGGGGGGGGGGRGGGGGGGGGGGGGGGGGEGGGGGGGGGGGGGGGGGGGGGGGGGGGGG |
| | | | GGGGGGGGKGGGGGGGGGGGGGGGGGGGGGGGGGGGGGGGGGGGGGGGRGGG-GGGGGGGGGGGGGGGG |
| 44 | Q | 0.972 | # QQQQQQQQQQQQQQQQRQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQ-QQQQQQQQQQQQQQQQ |
| 45 | H | 0.776 | # HYYHYYYYYYHYHHHHHYHHHYHLHHHYHHHYYYHYHYHHYHHHYHHHYHHYHNHHHHHHHHHHHHYHHH |
| | | | YYYHYHYHHHHHYHHHHHYHHYYHHHHHHHHHHHHHHHHHYYHYHHHHHHHH-HHYHHHHYHYYHHYYH |
| 46 | I | 0.986 | # IIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIXIIIIIIIIIIIIIIII |
| 47 | Y | 1 | # YYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYY |
| 48 | E | 0.915 | # EEEEEEEEEEEEEEEDEEEEEDDEENEEDEEEAEEEEEEEEEEEEEEEEEEEEEEEEEEEEEEEEEEEEE |
| | | | EEEEEEEEKEEEEEEEEEEEEEEEEEEEEEEEEEEEEEEEEEEEEEEEEEEEEEEEEEEEEEEEEEEEE |
| 49 | T | 0.986 | # TTTTTTTTTTTTTTTTTTTTTTTTTTTTTTTTTTTTTTTTTTTTTTTTTTTTTTTTTTTTTTTTTTTTTT |
| | | | TTTTTTTTTTTTTTNTTTTTTTTTTTTTTTTTTTTTTTTTTTTTTTTTTTTTTTTTTTTTTTTTTTTTT |
| 50 | Y | 0.986 | # YYYYYYYYYYYYYYYYYYYYYYYYYYYYYYYYYYYYYYYYYYYYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYSYYYYY |
| 51 | G | 0.927 | # GGGGGRGGGGGGGG6GGGGGGGEGGGGGGGGGGGGGGGGGGGGGGGGGGGGGGGGGGGGGGGGGGGGGGG |
| | | | GGGGGGGGEGGGGGGGGGGGGGGGGGGGGGGGGGGGGGGGGGGGGGRGKEGGGGGGGGGGGGGGGGGGG |
| 52 | D | 0.961 | # DDDDDDDDDDDDDDDDDDDDDDDDDDDDDDDDDDDDDDDDDDDDDDDDDDDDDDDDDDDDDDDDDDDDDD |
| | | | DDDDDDDDNDDDDDDDDXDDDDDDDDDDDDDDDDDDDDDDDDDDDDDDDDDDNDDDDDDDDDDDDDDDD |
| 53 | T | 0.986 | # TATTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTT |
| 54 | W | 0.952 | # WWWWWWWWWWWWWWWWWWWWWW$WWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWGWW$$WWWWWWWWWWWWWWWWWWW |
| 55 | A | 0.756 | # ATAATAAAATTATAATAAATAAAATAAEAAAATATTTAAAEVAATAATAAPAAAAAAAAAAAAAAAVAAE |
| | | | PATAAAAAATAAAAAAAAAAAATAAAAAAAAAAAAATAAAATAATTTTAAAAAAAAAAAAAAAATTAAP |
| 56 | G | 0.975 | # GGGGGGGGGGGGGGGGGGGGGGEGGGGGEGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGG |
| 57 | V | 0.986 | # VVVVVVVVVVVVVVVVVVVVVVVVVVVVVVVVVXVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVV |
| 58 | E | 0.913 | # EEKEEGEEEEEEEEEEEEEEEEEEEEEEEEEEEGEEEEEEEVEEEEEEEEEEEEEEEEEEEEEEEEEEEQ |
| | | | EEEEEEEEKEEEEEEEEEEEEEEEEEEEEEEEEEEEEEEEEEEEEEEEEEEEEEEEEEEEEEEGEEEEE |
| 59 | A | 0.972 | # AAAAAAAAAAAAALAAAAAAAAAAAAAAAAAAAAAAAAAAAAAAAAAAAAAAAAAAAAAAAAAAAAAAAA |
| | | | AAAAAAAAAAAAAAAAAAAAAAXAAAAAAAAAAAAAAAAAAAAAAAAAAAAAAAAAAAAAAAAAAAAAA |
| 60 | I | 0.845 | # ILLILILIIIIIIIIIIIILIIIIILIIIIILIIIILIIIIIIILIIILLLIIIIIIIIIIIIIIIIIII |
| | | | LIIILLIIIIIIIIIIILIIIIIIIIIIIILIILILIIIILIIIIIIILLLIIIIIIIIIIILILIIII |
| 61 | I | 0.938 | # IIVITIIIIIIIIIIIIIIIIIIIIIIIIIIIIIIIIIIIIIIIIIIIIIIIIIIIIIIIIIIIIIIIII |
| | | | IIIIIMIIIIIIIIIIIIIIIIIIIIIIIIIIIIIIIIIIIVIIIIIIIIIIIIIIIIIIIIVIIIIII |
| 62 | R | 0.966 | # RRRRRRRRRRRRRRRRRRRRRRKRRRRRRRRRRRRRRRRRRRRRRRRRRRRRRRRRRRRRRRRRRRRRRR |
| | | | RRKRRRRRKRRRRRRRRRRRRRRRRRRRRRRRRRRRRRRRRRRRRRRRRRRRRRRRRRRRRRRRRRRRR |
| 63 | I | 0.732 | # ITIITIIVVIITIIIIIIIMIVIIISIIIITIIIIIMIIIIIIIMIIITISIIIIIIIIIIIIIIIIIII |
| | | | STIIMIIMIISIIIIIITIMTIIIITIIIIIMIIISIIIMMITIIIIISIIIIIIIIIIIIITMIIIIT |
| 64 | L | 1 | # LLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLL |
| 65 | Q | 0.986 | # QQQQQQQQQQQQQQQQQQQQQQQQQQQQQQQQQQQQQQQQQQQQQQQQQQQQQQQQQQQQQQQQQQQQQQ |
| | | | HQQQQQQQQQQQQQQQQQQQQQQQQQQQQQQQQQQQQQQQQQQQQQQQQQQQQQQQQQQQQQQQQQQQQ |
| 66 | Q | 1 | # QQQQQQQQQQQQQQQQQQQQQQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQQQQQQQQQQQQQQQQQQQQQQQQQQQ |
| 67 | L | 0.961 | # LLLLLLLLLMLLLPLLLLLLLLLLLLLLLLLLLMLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLL |
| 68 | L | 1 | # LLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLL |
| 69 | F | 0.949 | # FFFFFFFFFFFFFFFFFFFFFFFFFFFFFLFFFFFFFFFFFFFFFFFFFFFFFFFFFFFLFFFFFFFFFF |
| | | | FFFFFFFFFFFFFFFFFFFFFFFFFFFFFFFFFFFFFFFFFFFFLFFFFFFFLFFFFFFFFFFFFFLFF |
| 70 | | 0.966 | # IIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIITIIIIIIIITIIIIIIIIIIIIIIIIIIIIITIIIIII |
| 71 | H | 0.972 | # HHHHHHHHHHHHHHHHHHHHHHHHHHHHHHHHHHHHHHHHHHHHHHHHNHHHYHHHHHHHHHHHHHHHHH |
| | | | HHHHHHHHHHHHHHHHHHHHHHHHHHHHHHHHHHHHHHHHHHHHHHHHHHHHHHHHHHHHHHLHHHHHH |
| 72 | F | 0.943 | # FFFFFFFFFFFFFFFFFFFFFFFFFFFFFFFFFFFFFFFXFFFFFFFFFFFFFFFFFFSSFFFSSFFFFF |
| | | | FFFFFFFFFFFFFFFFFFFFFFFFFFFFFFFFFFFFFFFFFFFFFFFFFFFFFFFFFFFFFFFFFFFFF |
| 73 | R | 0.961 | # RRRRRRRRRRRRRRRRRRRRRRKRRRRRRRRRRRRRRRRRRRRRRRRRRRRRRRRRRRTTRRRRRRRRRR |
| | | | RRRRRRRRRRRRRRRRRRRRRRRRRRRRRRRRRRRRRRRRRRRRRRRRRRRRRRRRRRRRRRRRRRRRR |
| 74 | I | 0.972 | # IIIIIIIIIIIIIIIIIIIIIIIIIIIIIIIIIIIIIIIIIIIIIIIMIIIIIIIIIIIIIIIIIIIIII |
| | | | IIIIIIIIIIIIIIIIIIIIIIIIIIIIIIIIIIIVIIIIIIIIIIIIIIIIIIIIIIIIIIIIIIIII |
| 75 | G | 0.911 | # GGGGGRGGGGGGGGGGRGGGGGKGGGGGRGGGGGGGGGGGGGGGGGGGGGGGGGGGGGGGGGGGGGGGGG |
| | | | GGGGGGGGEGGGGGGGGGGGGGGGGGGGGGGGGGGGGGGGGGGGGGRGKRGGGGGGGGGGGGGGGGGGG |
| 76 | C | 1 | # CCCCCCCCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCC |
| 77 | Q | 0.692 | # QRHQRRRRHHRQRRQQRQQQQRRQQRRQRRQRRHQQQRQRQQRRQQQRQQRRQHQQQQHQHHQHHRRQRQ |
| | | | RQRRQRRRRHRQQRRRRQHRRRQQRRQQQQQRQQQQQQQQRHQQRRRRQQRQRRQQRQRQRQRRRRQRQ |
| 78 | H | 1 | # HHHHHHHHHHHHHHHHHHHHHHHHHHHHHHHHHHHHHHHHHHHHHHHHHHHHHHHHHHHHHHHHHHHHHH |
| | | | HHHHHHHHHHHHHHHHHHHHHHHHHHHHHHHHHHHHHHHHHHHHHHHHHHHHHHHMHHHHHHHHHHHHH |
| 79 | S | 1 | # SSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSS |
| 80 | R | 1 | # RRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRR |
| 81 | I | 0.961 | # IIIIIIIIIIIIIIIIIIIIIIIMIIIIIIIIIIIMIIIIIIIIIIIIIIIIIIIIIIIIIIIIIIIIII |
| | | | IIIIIIIIILIIIIIIIIIIIIIIIIIIIIIIIIIIIIIIIIIIIIIIIIIIIIIIIIIIIIIIIIIII |
| 82 | G | 0.941 | # GGGGGGGGGGSGSGGGGGGGGGSGGGGGGGGGGGGGGGDGGGGGGGGGGGGGGGGGGGGGGGGGGGGGGG |
| | | | GGGGGGGGGGGGGGGGGGGGGGGGGGGGGGGGGGGGGGGGGGGGGGGGGGGGDGGGGGGGGGGGGGGGG |
| 83 | I | 0.949 | # IIIIIIIIIIVIIIIIIIIIIIIIIIIIIIIIIIIIIIIVIIIIIIIIIIIIIIIIIIIIIIIVIIIIII |
| | | | IIIIIIIIIIIIIIIIIIIIIIIIIIIIIIIIIIIVIIIIIIIIIIIIIIIIVIIIIIIIIIIIIIIII |
| 84 | T | 0.613 | # TTTTIITIIITIVSTT-TITITTXTN-FTTI-TXITTTLTTITTTTIITIITLIILIIIIIIIIIITTTT |
| | | | TTTTTITTIIITTGTIIT-ATITITTTTTIITTIT-TITTTI-ITTTTTIIITITIIIIITITIITITT |
| 85 | Q | 0.537 | # RRRRPPQRRRY-PPRPIPQPQRRQPP-RRYQ-RRQQRPRRRRPPPPQQPRQRRRRRRRRRRRRRRQRRQR |
| | | | PRRQRLPRQRRRLXQQPRQPPRRQQRRQQPQRQRQQQQPPRRQRTTTTQ-RQRRRQRRQ-RQQQQPRQR |
| 86 | Q | 0.732 | # QQQQQQQQQQS-QQQVQQQ-VQQVQQ-QQQQQQRRRQQRQQQQQRRRQRRQQ-QQQQQQQQQQQQRQQQQ |
| | | | QQQQQQQQQQQQQQQQQQRQQQQQQQRRRRRQRRRRRRRRQRRQRRRRRRQQQQQQQQQRQQRQQQQQQ |

TABLE 8C+TL,56

| | | | |
|---|---|---|---|
| 87 | R | 0.831 | # RRRRRRRRRRTRRRRRRRRRRRRRRRMRGKRRRSRRRRRRRRSSSSSRSSRRRRRRRRRRRRRRRRRRRR |
| | | | RRRRRRRRKRTRRXRRRRRRRRRRRRRRRRRRRSRRRRRRRRRRRRRRRRRRRRRRRRRRRRRRRRRRR |
| 88 | R | 0.855 | # RRRRRGRRGRGRGRRRSRRRRRRRRRRRRRRRRRRRRRRRRRRRRRRRRRRRRRRTRTRRRRRRRRRRRR |
| | | | RRRRRRRRKRRRRRRRRRRRRRRARRRRRRRRRRRRRRRRRRRRRGRRRRRARRRARRRRRARRRRRRR |
| 89 | A | 0.758 | # ATAAAAAAATEATAAATAAPAAAAAAAAAAAAAATTAAAAAATSAAAAAASATAAAAAAAAAAAAAAAAA |
| | | | TAAAATAPAAAAAAAAAATATAARAAAAAAAAAAAATAAAAASAAAAAATARAAARAASTARAATATAA |
| 90 | R | 0.944 | # RRRRRRRRRRNRGRRSRRRRRRRRRRRRRRRRRRRRRRRRRRRRRRRRRRRRRRRRRRRRRRRRRRRRRR |
| | | | RRRRRRRRKRRRRRRRRRRRRRRRRRRRRRRRRRRRRRRRRRRRRRRRRRRRRRRRRRRRRRRRRRRRR |
| 91 | N | 0.919 | # NNNNNNNNNNKNNNNNNNNNNNNNNNNNNNNNNNNNNNNNNNHNNNNNNNNNNNNDDNNNNNNINNNNNN |
| | | | NNNNNNNNNNNNNNNNNNNNNNNNNNNNNNNNNNNNNNNNNNNNNNNNSNNNNNNNNNNNNNNNNNNNN |
| 92 | G | 0.947 | # GGGGGGGGGGRGGGGGGGAGGGGGGGGGGGGGGGGGGGGGGGGGGGGGGGGGGGGGGGGGGGGGG6GGGG |
| | | | GGGGGGGGKGGGGGGGGGGGGGGGGGGGGGGGGGGGGGGGGGGGGGRGGGGGGGGGGGGGGGGGGGGGG |
| 93 | A | 0.854 | # AAAAAAAAAAAAASAAAATAAAAAASAVAAAASASAAAAAAAAAAAAAAAAAAAAAAAAAAAADAAAAAA |
| | | | AAAAAAAAAAAAAAAAATAAAAAAAAAAAAAAAAAAASAAASASAAAAATSAAAAAAASAAAASAAAAA |
| 94 | S | 0.849 | # SSSSSSSSSSSSSSSSSSFSSSSSSSSSSSSGSGSSSSSSSSGNRRSSRSSSSSSSRSRSSSSRSSSSSS |
| | | | SSSSSSSSSSSSGSSSSSSSSSSSSSSSSSSSSSSGGSSSSSSSSSSSSGSSSSSSSSSSSSSSSSSSS |
| 95 | R | 0.913 | # RRRRRRRRRRRRRRRRRRARRRRRRRRRRRRRRRRRRRRRRRRRRRRRRRRRRRRRSTTTRRRRRRRRRR |
| | | | RRRRRRRRKRRRRRRRRRRRRRRRRRRRRRRRRRRRRRRRRRRRRRRRRRRRRRRRRRRRRRRRKRRRR |
| 96 | S | 0.957 | # SSSSSSSSSSSSSSSSSSSSSSSSSSSSSSSSSSSSSSSSSSSSSSSSSSSSSSSSSSSSSSSSSPSSSS |
| | | | SSSSSPSSSSSSSSSSSSSSSSSSSSPSSSSSSSSSSSSSSSPSSSSSSSSSSSSSSSSSSSSSSSSSS |
| 97 | | 0.917 | # Y$$$$$$$$$$$$$$$$$$$L$$$$$$$$$$$$$$$$$$$$$$$$$$$$$$$$$$$Y$YYL$$YY$$$$$ |
| | | | $$$$$$$$$$$$$$$$$$$$$$$$$$$$$$$$$$$$$$$$$$$$-------$$$$$$$$$$$$$$$$$$ |

SAMPLE #: 378

| | | |
|---|---|---|
| | Nef | |
| AA# | ConS, entropy | Raw Data |

| | | | |
|---|---|---|---|
| 1 | M | 0.985 | # MMMMMMMMMMMMMMMMMMMMMMMMMMMMMMMMMMMMMMMMMMMMMMMMMMMMMMMMMMMMMMMMMMMMMMMMMMM |
| | | | MMMMMMMMMMMMMMMMMMMMMMMMMMMMMMMMMMMMMMMMMMMMMMMMMMMMMMMMMMMMMMMMMMMMMMMMMMMMMMMMMMMMM |
| | | | MMMMMMMMMMMMMMMMMMMMMMMMMMMMMMMMMMMMMMMMMMMMMMMMMMMMMMMMMMMMNMMMMMMMMMMMMM |
| 2 | G | 0.994 | # GGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGDGGGGGGGGGGGGGGGGGGGGGGGGGGGGGGGGGGGGGGG |
| 3 | G | 0.91 | # GGGGGGGGGGGGGGGGGGGGGGGGGGGGGGGGGGGGGGGGGGGGGGGGGGGGGGGGGGGGGGGGGGGGGGGAGGG |
| | | | GGGGGGGGGGGGGGGGNNGGGGGGGGGGGGGGAGGNGGGGGGGGGGGGGGGSGGGGSGGGSGGGGGGGGGGGGGGGGGGGGGGGG |
| | | | GGGGGGGGGGGGSGGGGGGGGGNGGGGGGGGNGGGGGSGGGGGGGGGGSGGSNGGGGGGGGGGGGGGGGGGGGG |
| 4 | K | 0.943 | # KKKKKKKKKKKKKKKKKKKKKKKKKKKKKKKKKKKKKKKRKKKKKKKKKKKKKKKRKKKKKKKKKKKKKKKKKKK |
| | | | KKKKKKKKKKKKKKKKKKKKKKKKKRKKKKKKKKKKKKKKKKQKKKKKKKKKKKKKKKKKKKKKKKKKKKKKKKKKKKKKKKKKK |
| | | | KKKKKKKKKKKKKKKKKKKKKKKKXKKKKKKXKKKKKKKKKKKKKKKKKEKKKKKKKKKKKKKKKKKKKKKKKK |
| 5 | W | 0.899 | # WWWWWWWLLW$WWWGWWWWWWWWWWWWWWWWWCWWWWWWGWWWWWGWWWWWWWWWWCWWWWWWRWWWWWWWWWWW |
| | | | WWWWWWWWWWWWWWWLWWWWWWWWWWWWWWWWWWWWWWWWWWWWWWWWWGWWWWWWWWWWLWWWWWWWWWWWWWWWWWWWWWWWW |
| | | | WWWWWWWWWWLWWWWWWWWWWWWWWWWWWWWWWWWWWWWWWWWWWCWWWWWLWWWWWWWWWWWWWWWWWGWCWW |
| 6 | S | 0.988 | # SSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSXSSSSSSSSSSSSSSSSSSSSSSSSSSSSSSSSSSSSSSSSSS |
| 7 | K | 0.973 | # KKKKKKKKKKKKKKKKKKKKKKKKKKKKKKKKKKKKKKKKKKKKKKKKKKKKKKKKKKKXKKKKKKKKKKKKKKK |
| | | | KKKKKKKKKKKKKKKKKKKKKKKKKKKKKKKKQKKKKKKKKKKKKKKKKKKKRKKKKKKKKKKKKKKKKKKKKKKKKKKKKKKKK |
| | | | KKRKKKKKKKKKKKKKKKKKKKKKKKKKKKKKKKKKKKKKKKKRKKKKKKKKKKKKKKKKKKKKKKKKKKKKKK |
| 8 | R | 0.519 | # SSLRIRSRRRRRRRHRSRRNSNRRRLRRSSSR-GRRRSS-S-CPSRCSSSHRCHSS-CSXSCCRSCRLRRRSRRS |
| | | | RRRSRSRSNSSRRSCR--RHCCRSRHSRRCCSCRRCSRRSSSRRRRRSCSRSSSRRRRRPRRRRRRSRRNSRSSRRSRCRLSRRC |
| | | | -LSPRR-SCRRSSRSXRNNRSSSLMSRSSSRSSRRPHSSCIRRSRRRRLRRCSRNRRRSR-RRRRRRRRCRHRR |
| 9 | S | 0.571 | # SR-SRSSSSSGKSXSSSSRRSSRSSSSSSRSS-SXISSS-R-SKSI-SRKSSSSSS-SSMSTSSSSM--SS---I |
| | | | SS-KKKISSSSSSSS---SRSS-S-SSSSSSSSSSKSSSSRRSSSMSSSSVSSSSK-S-S-SSTKSRS--RKSKKSS-SSRSSSK |
| | | | T-SNS---SSSSKSSSSSRKSSS-RSSSSSSSSSSSSSSS--SSSSSS--RG-SSSGSSS--SSSSSGSGGSSS |
| 10 | V | 0.35 | # VKKE-VIGG--RGG-MVGGVVV$E--KGVVVTLGGGVIVLGM-LVR-IV-MMMMIMLVI-M-MVV--EVGVVVVG |
| | | | VV-MGLGKMMVMLVVVMMKG-GVVVE-RGMALP-V-VVLMG-LV--VIV--VVVVMKLLVLMVGEVIVLV-VM--LIG---VVMG |
| | | | LRMFMMLM-LMMVVVVVVVAXV---MGIVVMXKVMVFVV-GVVVGFV--IV-VIVIVAV-LVVVVVVVVVFFVA |
| 11 | V | 0.315 | # VISD-VVGG--VGV-PIGVIVVVD--IPVGVGGFVGVVVGVA-GVSAFV-FVVGFFRVVAI-VTV-DIIGNTVTG |
| | | | LP-SDGAISSIGVVVAIIVD-DTIVG-RGKIVV-VVVFSIVVGG-AVIVNGIIVVDVPGPSGVGDVFVGP-PGLLGRG-V-VVFG |
| | | | GGGPGGGV-GFGISIIIITSIV---IVVIVASIVGPPVVGAPVVLPP-GPP-VPPPPPP-GPPPPPPPPPPPPP |
| 12 | G | 0.863 | # GGGKGRGGGG-EGGGGGGGGGGGRGGEGGGGGGGGGGGGGGGGGGEGGGGGGGGGGGGGGGGGGGGSGGGGGGGG |
| | | | KGGGSGGGGGGGGGGQGGEGGREGGGGEGGGGGEGGGGGGGGGGEGGGGKGGGGGGEGGGGGGGGGGGGGGGGGEGGGGEGGGGG |
| | | | GGGGGGGGGGGGGGGGLGGGGG-GGGGGGGGGGGGGGGGGGGGGGGGGGGGGGGGGGGGGGGGGGGGGGGGGGG |
| 13 | W | 0.969 | # WWWWWWWWWW-WWWWWWCWWWWWWWWWWWWWWWWWWWWWWWWWWWWWWWWWWWWWWWWWWWWWWWWWWWWWWWWW |
| | | | WWWWWWWWWWWWWWWCWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWSWWWWWWWWWWWWWWXWWWWWWWWWWWWWWWWWWWWWWWWWWWWWWWWWWWWWWWWWW |
| 14 | P | 0.512 | # PPPPSPPAAS-PPSQSPSPPPPSSSSPSPSPPPPPSPPPPPFSHPPPPPAPPPSPPSPPXPSPFPPPAPNPPSSP |
| | | | PSPDPAPPTPPPPPPSPPPAPPSPPPPPPPPPPSPPPPSPPRPPSSPPPPPPPHPTPPNPSSPPPPSPPQSPASSIPPPPPPPAS |
| | | | PTSAPPPPPSSPPPPPPPIPPPPSPPPPPPSPPPPAAPPHAAPPPDNPPDNPPDNDNNNPPNNDNNNNNDDDNN |
| 15 | T | 0.584 | # ATDAATATTT-KATSTATTAAATTTRTTAAAATATTTTATTTAATTTTAASSSIGATTNXAATTATSTTAATTTA |
| | | | ATAASAATTATATATAAAATAAATAASARTAAATATTTATTAAAANTTTAATARKTAAHKAATSAKAATTAAITAVTAATAATTT |
| | | | TAAAAATTAARAAAAATTTATAATTTTAATXAAAATAAAKTTTAKTTANATAATTTATTANTAATATTSTTTTT |
| 16 | V | 0.758 | # IVIVVIVVVV-VVVVIVVVVVVVIVVVVVVVVVVVVVVVVVVVVVVVVVVVVVIVVVVVVIVVVIVVVVVVDIII |
| | | | VIVIVIIVIIVVVVVVVVVIVVVIVVVVVIVVIVVVVVVVVVVVIVVVVVVIIVVVVIVIVVVVVVVVVIVIIIVIXVVVIIVVV |
| | | | VVIVVVVVVVVVVIVVVIVVVIVXVVVVVILVVVVIVIVVVVVVVIIVVIIVVIIIIIIVVIIIIIIIIIIIII |
| 17 | R | 0.952 | # RRRRRKRRRR-RRRRRRRRRRRRRRRRRRRRRRRRRRRRRRRRRRRRRRRRRRRRRRRRRRRRRRRRRRRRRRAR |
| | | | RRRRRRRRRRRRRRRRRRRRRRRRRRRRRRRRRRRRRRRRRRRRRRRRRRRRRRRRRRRRRRRRRRRRRRRRRRRRRRRRRRRRR |
| | | | RRKRRRRRMRRRRRRRRRRRRRRRRRRRRRRRRRRRKRRRRRRRRRRRRRRRRRRRRTRRRRRRRRRRRRRRRR |
| 18 | E | 0.921 | # EEDEEEEEEE-EEEEEEAEEEEEEEEEEEEEEKEEEEEE$EEEEEEEEEEEEEEEEEEEDDEEEEEEEEEEEEEE |
| | | | EEEEEEEEEEEEEEEEEEEEEEEEEEEEEEEEDEEEEEEEEEEEEEEEEEEEEEEEEDEEEEEEEEEEEEEEEEEEEEEEEEEEE |
| | | | EEEEDDEEEEDEEEEEEEEEEEEEEEEEEXEEEEEEEEEEEEEEEEEEEEEEEEEEEEEEEEEEEEEEEEEEEE |
| 19 | R | 0.889 | # RRRRRKRRRR-RRRRRRKRRRRKRRRRRRRRKRRRRRRRRRRRRRRRRRRRRRKRRRRRXRRRRRRRRKRRRRRR |
| | | | RRRRRRRRRRRRRRRRRRRRRRRRRRRRRRRRRRRRRRRRRRKRRRRRRRRRRRRRKRKRRRRRRRRRRRKRKRRRRKRRRRRRR |
| | | | RRRRRRRRRRRRRRRRSRRRRRRRRRRRRKRRKRRRRRRRRRRRRRRRRRRRRRRRRRRRRKRRRRRRRRRRRR |
| 20 | M | 0.913 | # MMMMMMMRMM-MMMMIMMMMMMMMMMMMIMMMMVMMMMMMMMMMMIMMMMMMMMMMMMMXMMMMMMMMMMMMMMM |
| | | | MMMMMMMMMMMMMMMMMMMMMMMMMMMMMMMMMMIMMMMMMMMMMMMMMMMMMMMMMMMMMMMMMMMMMMMMMTMMMMMMMMMMM |
| | | | MMMIMMMMMMNIMMNMIMNMMMMNMMMMMMMMMMMMMMIMMMMMMMMMMMMMMMMMMNMMMMMMMMHMMMMNMM |
| 21 | R | 0.7 | # RKRRERKRRR-EKRRRREERRRRRRRNRQKKRRRKRRKRKKRQRRIRRRRKKKKRKRRRIRQRRKKSRSRRRRRR |
| | | | RRRRSQRRRKRRRRRRQQRRRRRRRRREQKKKRRRRRRERRRKKRRRKKRRKRRRRKRRRQRRRRTRKRKERRRQRRQRRRRRRR |
| | | | RRHRQQRRRQQRRRRRERRRRRRRRRRRRRRRRRERRRRERRERRRRERRRRRRRRRTRERRRRRRRRRRRRRR |
| 22 | R | 0.607 | # RQQRRQTRRR-RRARQVRRARRRARRRARPRGRRRRRRRRQQQRRQRRRKRRRRRRARRXRAQRREERRRQQRRK |
| | | | RRRPEPERQRRRRRRPRRQQPRRRRRARRPRQRRRRRRVAQPQREQRRRERERRQRRRQARRRRRRRRRRKAKQQPRQRRRARAP |
| | | | RRRARRRRQRQQRQRRKRRRRXAARRQARRRRRARRRRRRRARRPRRRRRRAAGRRRRRRRRRREERRRRRRRR |
| 23 | A | 0.633 | # AADTAAAAAA-TIADTAAAAAAAAAAANSAAATAAATTATAAAAAAAAAAAAATATAAATAAAAAPPTATAAAAA |
| | | | TAATPAPAAAATAAAAAAEAAAAAATAAAPAAAAAATAVAATTAPAAVAPTPAAATATAAAATTAAAAVAAATATAAAAAAAAAA |
| | | | AAATAAAATAAPTTAAATAAAAXAATAATAAAAATTTTATAEATANAATTTDLTTTTTAATTTTPPTAAANNTT |
| 24 | E | 0.839 | # EEEEEEEEEE-EEEEEEEEEEEEEEEEEEGEEEEEEEEQEEEEEEEEEEEEEEEEEEEEEEEEEEEEEEEEEEEE |
| | | | EEEEEEEEEEEEEEEEEEPEEEEEEEEEEEEEEREEEEEEEEEEAEEEEETEEEEEEEEEEEEEEEEEEEEEEEEEEEEEEEEEE |
| | | | EEDEEEEEEEEEEEEEDEEEEEGEEEEEXEDEEEEEEEEEEPEEEEEEDEEEPEAEEEEEDEEEKEEEEEEEEE |
| 25 | P | 0.882 | # PPPPPPPPPP-PPPPPPPPPPPPPPPPPPPPPPPPPPPPPPPPPPPPPPPPQPPPPPPPPPPPPPPLPPPPPPPP |
| | | | PAPPPPPPPPPPPPPPPPAPPPPPPPPPPPPPPAPPPPPPPPPPPPPPPPPPPPPPPPPPPPPPPPPPPPPPPPSPQPPPPPPPP |
| | | | PPPPPPPPPPPPPPPPPPPPPPVPPPPPPPPPPPPPPPPPPAPPPPXPQPTPAPPPPPPPQPPPPPPPPPPPPP |
| 26 | A | 0.935 | # AAAAAAAAAA-AAAAAAAAAAAAAAAAAAAAAAAAAAAAAAAPAAAAAAAAAAAAAAAAAAAAAAAAAAAAAATA |
| | | | AAAAAAAAAAAAAAAAAAAAAAAAAAAAAAAAA-AAAAAAAAATAAAAAAAAAAAAAAAAAAAAAAAAAAAAAAAAAAAAAAAAA |
| | | | AAAAAAAAAAAAAAAAAAAAAAAAAAAAVAAAAAAAAAAAAAAAAAAAAAAAAAAAAAAAAAAAAAAAAAAPAA |
| 27 | A | 0.943 | # AAAEAAAAAA-AAAAAAAAAAAAAAAAAAAAAAAAAAAAAAAAAAAAAAAAAAAAAAAAAAAAAAAAAADAAAAA |
| | | | AAAAAAAAAAAAAAAAAADAAAAAAAAAAAAAA-AAVAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAEAAAAAAAAAAAAASAAAAA |
| 28 | D | 0.595 | # DED-EEDEDD-DDEDEDEEDDDDDDDPVDEEEDDEEDDDDDVADDADDDDDDDDDDADAEVXDDEDDAE-DDVEE |
| | | | E-DDDKVDDDDEEDDDDD-DDEDDDDDEPEDDD-EEEDDEDDEEDEEDEVEEDDEDDDDDEDDDEDEDDEEVDEVEDAEEDDDDA |
| | | | DDHEAADDEEEADADDDDDDKLXDDDDADEDDDDDDDDDEDDEEDEEDDEATE-EEEEDDEEEEEEEEEEEEEE |
| 29 | G | 0.958 | # GGGGGEGGGG-GGGGGGGGGGGGGGGGGGGGGGGGGGGGGGGGGGGGGGGGGGGGGGGGGGGGGGGGGGGGGGGG |
| | | | GGGGGGGGGGRGGGGRGGGGGGGGGGGGGGGGG-GGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGGGG |
| 30 | V | 0.963 | # VVVVVVVVVV-VVVVVVVVVVVVVVVVVVVVVVVVVVVVAVVVVVVVVVVVVVVVVVVVVVVVVVVVVVVVVVVV |
| | | | VVVVVVVVVVVVVVVMVVVVVVVVVVVVVVVVV-VVVVVVVVVVVVVVVVVVVVVVVVVVVVVMVVVVVVVVVVVVVVVVVVVVV |
| | | | VVVVVVVVVVVVVVVVGVVVVVVVVVVVVVVVVVVVVVVVVVVVVVVVVVVVVVVVVVVVVVVVVVVVVVVVVV |
| 31 | G | 0.967 | # GGGGGRGGGG-GGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGG-GGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGGGG |
| 32 | A | 0.966 | # AAAAAAAAAA-AAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAPAAAA-AAAAAAAAAAAAAAAAAAAAAAAAAAAAAAAAAPAAAAPAAAAAAAAAAAA |
| | | | AAAAAAAAAAAAAAAAAAAAAAAAAAAAAAAAAAAAAAAAAAAAAAAAAAAAAAAAAAAAAAAAAAAAAAAAAA |
| 33 | V | 0.78 | # AVVVVVVAAV-VVAAVVVVVVVVAVVVVVAVVAVVAVVVAVAVAAAAAVAVVVAAAVVVAAVVVVAVVVVAVVVV |
| | | | VVVAVAAVVVAAVAVVAAVVVVVAVAAAAAAVV-VVVVAVAVVVVVVVVVVAVAVAAAVVVAVVVAVAVVAAVVVVVVVVVVVAA |
| | | | VVAVVVVVVVVVVVAAVVVVVVVVVVVVVAVVVVVVVVVAVVVVVVVAVVVVVVVVVVVAVVVVAAAAVAAVAV |
| 34 | S | 0.984 | # SSSSSSSSSS-SSSSSSSSSSSSSSSSSSSSSSSSSSSSSSSSSSSSSSSSSSSSSSSSSSSSSSSSSSSSSSSS |
| | | | SSSSSSSSSSSSSSSSSSSSSSSSSSSSSSSSS-SSSSSSSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSFSSSSS |
| 35 | R | 0.905 | # RRRRRRRRRR-RKRRRRRRRRRRRRRRQRRRRRRRRRRRRRRRRRRRRRRRRRRRRRRRRRRRRRRRRRQRIRRR |
| | | | QRRRRPRRRRRRRRRRRRRRRRRRRRRRRRRRR-RRRRRLRRRRRQRRRRRRRRRRRRRRRRRRQRRRRRRRRRRRQRRRRRRRR |
| | | | RRPRRRRRRRRRRRRRRRRRRRQRRRRRRRRRRRRRRRRRRRRRRRRRRRRRRRRRRRRRRRRRRRRRRRRRRR |
| 36 | D | 0.978 | # DDDDDDDDDD-DDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDTDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDDDD |
| 37 | L | 0.971 | # LLLLLLLLLL-LLLLLLLLLLLLLLLLLLLLLLLLLLLLLLLLLLLLLLLLLLLLLLLLXLXLLLLLLLLLLLLL |
| | | | LLLLLLLLLLLLLLLLLLLLLLLLLLLLLLLLLPLLLLLLLLLLLLLLLLLLLLLLLLLLLLLLLLLLLLLLLLLLLLLLLLLLL |
| | | | LLLLLLLLLLLLLLLXLLLLLLLLLLLLLLLLLLLLLLLLLLLLLLLLLLLLLLLLLLLLLLLLLLLLLLLLLL |
| 38 | E | 0.817 | # EEEAEEEEEE-EEEEEEEE0EEEEEEEAEE$EEEEEEEEEEEGEEEEEEEEEEAEEEEEEDEEEEKEEEGEEEEE |
| | | | GEDEEEEEEEEEEEEDEEEEADEEEDEDEEEEEEEEEEEDEEEEEGEEEEEEEEEEEEEDEEEEAEEEEEEEEEEAAEEEEEEEE |
| | | | KEEEEEEEEEEEEEEEEEEEEEGGEEEEGEEEEEEEEEEAEGEEEEEEEEEEEADEEEEEEEAEKEEEEEEEAE |
| 39 | K | 0.659 | # RKKRKKKKKK-KKRKKKKKEKKKKKKKKKKKKKKKRKKRKKKKKKKRKKKKKKRKKRKRKKKRKKKKRKKKKRKK |
| | | | KKRRKKKKKKKRKKKQKKKKAKAKAKRKKKKKKKKKKKKKKKKKKKKKKKKKRKKKKKKKKRKKKKKKKKKRKKKKKKKKKRKRR |
| | | | KRKRKKKKKKKRRKKKKRRRKKXKKKKKKKKRKRRRKKRKRTKKRQQKKQQKQQQQQQQKKQQQQQQQQQQQQQ |
| 40 | H | 0.735 | # HHHHYHHHHH-HHHHHHHHHHHHYHHHHHHHHHHHHHHHHHHHHHHHHHHHHHHHHHHHHHHHHHHHHHHYYHHH |
| | | | HNHHNNHNHHHMMHHRNHNYRNRMRYYYHHHHNHYMYHYHHHMHHHHHHMMYNHNHNHHHHMNHHMHHHHYNHHYMHNHNHMHYH |
| | | | HMNHHNNHHHHHHHYYHHHHHHHHXYHHHHHHHNHHNNHHRRHHHRRYHRRHHRRRHHRYHRRRRRRRRHRRRR |
| 41 | G | 0.952 | # GGGGGGGGGG-GGGDGGGGGGGGGGGGGGGGGGGGGGGGGGGGGGGGEEGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGGGGGGGGGGRGGGG |
| | | | GGGGGGGGGGGGGGGGGGGGXGGGGGGGGGGGGGGGGGGGGGGGGGGGGGGGGGGGGGGGGGGGGGGGGGGGGG |
| 42 | A | 0.977 | # AAAAAAAAAA-AAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAGAAAAAAAAAAA |
| 43 | I | 0.609 | # LIIIVIVIII-IIIIIIIIIIIIIIIIIIIEILIIIIIILIIIIIILIIIIIIIIIIIIIIIIIIIIIIILIIII |
| | | | IIILILLIIIIIIIIILLIIIIINILIIIILIIIIIIIIIIIIIIIIIIIILIIIIIIIVIIIIILIIIILIIIIVIIIIIIILI |
| | | | IILIIIIIIIIIIILIIIIIIIIIIIIIIIIIIIIIIIIIIIIIIIIIVIIIIIIVIIVLVIIIIIIIIIIIII |
| 44 | T | 0.966 | # TTTTTTTTTT-TTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTTTT |
| 45 | S | 0.714 | # SSSSSSSSSS-SNSSSSSSSSSSSSSSSSSSSSSSSSSSSSSSSSSSSSSSSSNSASSSSNSSSSSSSNSSNSSS |
| | | | SSSSSSTSSSSSSSSSSSSSNSSISNSSSSSSSGSNSSSSSSSSSSSSSSSSSSSNSSSSSSSSSSSSSSSSSSSSSSNSSSSSS |
| | | | SSSSSSSSSSSSSSSSSSSSSSSSSSSSSSSXSSSSSSSSSSSSSTISSITSSTTTTTTSSTTTTTTTITTTTT |
| 46 | S | 0.643 | # SSSSSSSSSS-SSSSSSSSSSSSSSSSSSSSSSSSSSSSSGSSSSSSSSSSSSSSSSNNSSSSSSSSSSSSTSSS |
| | | | SSSSSSSSSSSSSSSSSSSSSSNSNSSSSSSSSSSRSSSSSSSSSSSS-SSSSSSSSSSSSSSSSSSSNSSSSSSSSSSSSSSSS |
| | | | SSFSSSSSTSSSSNNNSSSSSSSSSSSSSSSSSSSSSSSSSSSSNSNSSNSSSSSSSSSSSSSSSSSSSSSSS- |
| 47 | N | 0.977 | # NNNNNNNNNN-NNNNNNNNNNNNNNNNNNNNDNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNNNNNNNNS |
| 48 | T | 0.916 | # TTTTTTTTTT-VTTTTTTTTLLTTTTTTTTTTTTTTTTTTTTTTT-TTTTTTTTTLTTTTTTTTTTTTTTTTTTT |
| | | | TTTTTTTTTTTTTTTTTTTTTTTTTTTTTTTTTT-TTTTTTTTTT-TTTITTTTTTTTTTTTTTTATTTTTTTTTTTTTTTTTTT |
| | | | TTTTTTTTTTTTTTTTTTTTATXTTTTTTTTTTTTTTTT-TTTTTTTTTTTTTTTTTTTTTTTTTTTTTTTTTT |
| 49 | A | 0.77 | # PAAAAA-AAA-AAAAAAAAP--AA-AAPAA-AAAAAAAAAVAAAA-AAAAA--AP-AAAAAAAAPAAAAPAAAAA |
| | | | A-AAAAAAAAAAATAAPPAAAAAAATAASAAAAA-AAAAAAAPAA-AAAAAPPAAAAPAAAAAAAAAAAAAPAAAAAAPPAAAAA |
| | | | AAPAAAA-AAAAAPAAAAAP-P-APAAAAAPAAAAAAAA-AA-AAAAAAAAAAAAAATAAAAAAAAAAAAAAA- |
| 50 | A | 0.529 | # AAAAAA-AAA--AAKATATA--NAXAATAA-AAATTTTAAATATAIATAEA--DA-AAAAAAAAATAAAATASAA |
| | | | A-ATAAATAAANTAAAAAAAAATTATAATAGAATTTAANAATAAAIVATAAAATAAAAAAAATAAAAAAASAATAAAAAAASTTA |
| | | | TAAAAAA-AAAATATTAAAA-A-AATTAAAATTAAAAAAIAT-AASSAAGSAASNSLSNAASDSSSSSSESSSS |
| 51 | N | 0.73 | # NTTTTNTTTN--TNNTSTTNNNNTNTTNNNATNTTNNNNNNNTNTNNNTTTAATTNTTTTNTTNTNTNTNNTNTN |
| | | | TNTNTNNNTTTTNNNTNNTNSTNNTNTTNTNTTNNNNTTNNNNTTNNNNSTNTNTTTNTNTTNNTNTTTTTTTNTTTNTNNNNNT |
| | | | NTTNTTTNTTTTNTNNTTTTNNNTTNNTTNNNNTNTNTNNSTDNNNNNNNNTTNNNNNNNNNNNNNNNNNNNNN |
| 52 | N | 0.966 | # NNNNNNSNNN-NNNNNNNNNNNNNNNNNNNSNNNNNNNNNNNNNNNNNNNNNNNNNNNNNNNNNNNNNNNNNNNN |
| | | | NSNNNNNNNNNNNNNNNNNNNNNNNNNNNNNNNNNNNNNNNNNNNNNNNNNNNNNNNNNNNNNNNNNNNNNNNNNNNNNNNNNNN |
| | | | NNNNNNNNNNNNNNNNNNNNNNNNNNNNNNNNNNNNNNNNNNNNNNNNNNNNNNNNNNNNNNNNNNHNNNNNNS |
| 53 | A | 0.928 | # AAPAAAAAAA-AAAAAAAAAAAAAAAAAAAAAAAAAAAAAAAAAAAAAAAAAAAAAAAAAAAAAAAAPAAAAAAA |
| | | | AAAAAAAAAAAAPAAAAANAAAAAAAAAAAAAAAAAAAAAAAAAAAAAAAPAAAAAAAAAAAAAAAAAAAAAAAAAAAAAAAAAA |
| | | | AAAAAAAAAAAAAAAAAAAAAAAAAAAAAAAAAAAAAAAAAAAAAAAAADAAAAAAAAAAAAAAAAAAAAAAAA |
| 54 | D | 0.699 | # ADDDTDADDD-NDDVADDADAADDDDDEDDADDDADDADDAADAADDDADADDDDDDDDDDDDDAAADADDDDAD |
| | | | DADADADDDDADNAADDDDDDTDADDDDDDADDNADDDDAADDDDADDDDDTDDADDADDDDDDDDDDDDADDADDDDADADADD |
| | | | ADDADDAADDDDADDDDDDDADADDDDDDADDDADDADDDDDAADATDDADDDAAAADADDAAATTAADADAAA |
| 55 | C | 0.885 | # CCCCCYCCCCXCCCCCCCCCCCCCCCCCCCCCRCCCCCCCCCCCCCCCCVCCCCCCCCCCCCCVCCCCCCCSCCC |
| | | | CCCCCCCCCCCCCCCCCCCCCCCCCCCCICCCCCCLCCCCCCCCCCCCLCCCCCCCCCSCCCCCCCRCCCVCCCCCCCCCCCCCC |
| | | | CCCCCCCCCCCCCCCCCCCCCCCCXCCCCCCCCCCCCCCCCCCCCCCLCCCCICCCCCCLCCCCCCCCCCCCCC |
| 56 | A | 0.904 | # AAAAAAVAAAAVAAAAAAAATTAAAAAAAAVAAAAAAAAAAAAAAVAAAAAVVAAAAAAAAAAAAAAAAAAAAAA |
| | | | AAAAAAAAAAAAPAAPAAAAAAAAAAAAAAAAADACAAAAAAAAAAAATAAAAAAAAAAAAAAAAAAAAAAAAAAAAAAAAAAAA |
| | | | AAAAAAAAAAAAAAAAAAAAAAAAXAAAAAAAAAAAAAATAAAAAAAAAAAAAAAAAAAAAAAAAAAAAAAAAV |
| 57 | W | 0.975 | # WWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWGWWWWWWWWWWWWWWWWWRWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWWWW |
| 58 | L | 0.916 | # LLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLVVLLLLLLLLLLLLLLLLLLLLLLLLLLILLLLLLLLLLLLLLLLLLLLLLLLLVLLLLLLLLLLLLLL |
| | | | LLPLLLLLVLLLLLLLLLLLLLVLLLLLLLLLLLLLLLLLLLLLLLLLVLVLLLLLLLLLVLLLLLLLLLLLLL |
| 59 | E | 0.938 | # EEEEEEGEEEEEEEEEEEEEQQEEEEEEEEEEEEEEEEEEEEEEEQEEEEEEEEENEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEQEEEEEEEAEEEEE |
| | | | EEEEEEEEEEEEEEEEEEEEEEEEEEEEEEEEEEKEEEEQEEEEEEEEEEEEEEEEEEEEEEEEEEEEEEEEEE |
| 60 | A | 0.972 | # AAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAPAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAPAAAAAAAAAAAAAAAA |
| 61 | Q | 0.943 | # QQQQQQQQQHQQQQQQQQQQQQQQHQQQQQQQQQQQQQQQQQHQQQQQQQQQQQRQQQQQQQQQQQQQQQQQQQQ |
| | | | QQQQQQQQQQQQQQQQQQQQQQQQQQQQQQQQQQQQQQQQQQQRQQQQQQQQQQQQQQQQQQQQQQQQQHQQQQQQQQQQQQQQQ |
| | | | QQQQQQQQQQQQQQQQQQQQQQQQQQQQQQQHQQQQQQHQQQQQQQQQQQQQQQQQQQQQQQQQQQQQQQQQQQ |
| 62 | E | 0.917 | # EEEEEEEEEEEEEEEEEEE-EEEEEEEEKEEEQEEEEEEQEEEEKEEEEEEEEEEKEEEEEEEEEEEEEEEEEEE |
| | | | EEEEEEEEEEEEEEEEEEEEEEEEEEEEEEEEEEEEKEEEEEEQEEEEEQEEEEEEEEQEEQEEEQEEEEEEEEEEQEEEEEEEE |
| | | | EEEEEEEEKEEEEEEEEEQEEEEEEEEEEEEEEEEEEEEEEEEEEEEEEEEEEEEGEEEEEEEEEEEEEEEEEE |
| 63 | E | 0.809 | # EEDEEEDEEEEEEEEDEEEEEEEEEEEKDEEEEDEEEEEEEEDDEEEDEEEEEDEEEEEXGDEEEEEEEEEEEEE |
| | | | DEDEEEDEEEEEDEDEEEEDDEGEEEEEEDEEEEEDEEDEEEEEEEDEEEEEEEEEEEDEEDEEEEEEDDEEEEEEEDEEEEEEE |
| | | | EDEEDEEEEEEDEEDDEDEEEDXXEEDXEEEDEEKEEEDEDEEEEEEEEEEDEDEEEEEEEEEEEEEEEEEEEE |
| 64 | E | 0.884 | # EEEEEEEEEEEEEEEEEEEEDDEEEEEEEEEEEEEEEEEGEEEEEEEEEEEEEGEEEEGEEEEEEEEEEEEEEEE |
| | | | EEEEEEEEEEEEEKEEEEEEEEEEEEEEEEEKEEEEEEEEEEEEEEEGKEEEEEEEEEGEEEEEEEEEEEGEEEEKEEEEEEDEE |
| | | | EEEEEEEEEEEEEEEEEEEEEEGEEEEEEGGEEEEEEEEEEEEEEEEEEEEEEEEEEEEEEEEEDGEEEEEEGE |
| 65 | E | 0.914 | # EEEEEEEEEEEEEEEEEEEEEEGEEEEEEEEEEGEEEEEEEEEEEEEEEEEEGEEEEEEXEEEEEEEEEEEEEEE |
| | | | EEDEEEDEEEEEEENEEEEEDEEEEEEEEEEEEEEEEEEEEEEEEEEEEEEEEEEEEEEEEEEEEEEEEEEEEEEKEEEEEEDEE |
| | | | EEEEEEEEEEEDEEEEEDEEEEEDEEDEEEEEEEEEEEEEEEEEEEEEEEEEEEEEEEEEEEEEEEEEEEEEEE |
| 66 | V | 0.973 | # VVVVVVVVVVVVVVVVVVVVVVVVVVVVVVVVVVVVVVVVVVVVVVVVVVVVVVVVVVVVVVVVVEVVVVVVVVV |
| | | | VVVVVVVVVVVVGVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVXXVVVVVVVVVVVVVVVVVVVVVGVVVVVVVVVVVVVVVVVVVVVVVVVVVV |
| 67 | G | D.978 | # GGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGRGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGGGG |
| 68 | F | 0.988 | # FFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFXFFFFFFFFFFFFFFFFFFFFFFFFFFFFFFFFFFFFFFFFFFFFFF |
| 69 | P | 0.994 | # PPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPP |
| 70 | V | 0.966 | # VVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVIVVVVVVVVVVVVVVVVVVVVVV |
| | | | VVVVVVVVVVVVVVVVVVVVVVVVVVVVVVVVVVVVVVVVVVVVVIVVVVVVVVVVVVVVVVVVVVVVVVVIVV |
| 71 | R | 0.769 | # KRRRKRKRRRRRRRRRKRKRRRKRRKKRRRRRRRRRKRRRRRRRRRRKRRRRRRRRRRKRRRRRKRKRRRKRKKK |
| | | | RRRRRRKRRRTRRRRKKKKRRRKRKRRRRRRRRRKKRTRRRRRRRTRKRRRRKRRKRKKRRRRRRRKRKRRRKRRRRTRRTTRRR |
| | | | KRRRRRTRRRRRRRRRQRRRRKRRRRRRRRRRRRRRKRRRRRKRTRRRRRKKRRRRRRRRRTRRRRRRTRKIRR |
| 72 | P | 0.973 | # PPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPLPPPPPPPPPPPPPPLPPPPPPPPPPPPPPPPPPPPPPPPPPPPP |
| | | | PPPPPPPPPPPPPPPPTPPPXPPPPPPPPPPPPPPPPPPPPPPPPPPPPPPPPPPPPLPPPPPPPPPPPPPPPP |
| 73 | Q | 0.955 | # QQQQQQQQQQQQQQQQQQQQQQQQQQQQQQQQQQQQQQQQQQQQQQQQQQQQQQQQQQQQQQQQQQQQQQQQQQQ |
| | | | QQQQQQQQQQQQKQQQQQQQQQQQQQQQQQQQQQQQQQQQQQQQQQQQQQQQQQQQQQQQQQQQQQQHQQQQQQQQQQQQQRQQQ |
| | | | QQQQQQQQHQQQQQQQQQQQQQQQQQQQQQQQQQQQQQQQQQQQQQQQQQQQQQQQQQQQQQQQQQQQEQQQQQ |
| 74 | V | 0.975 | # VVVVVVVVVVVVVVVVVVVVVVVVVVVVVVVVVVVVVVVVVVVVVVVVVVVVVVVVVVVVVVVVVVVVVVVVVVV |
| | | | VVVVVVVVVVVVVVIVVVVVVVVVVVVVVVVVVVVVVVIVVVVVVVVIVVVVVVVVVVVVVVVVVVVVVVVVVVVVVVVVVVVVV |
| | | | VVVVVVVVVVVVVVVVVVVVVVVVVVVVVVVVEVVVVVVVVVVVVVVVVVVVVVVVVVVVVVVVVVVVVVVVVV |
| 75 | P | 0.985 | # PPPPPPPPPP-PPPPPPPPPPPPPPPPPPPPPPPPPPPPPPPPPPPPPPPPPPPPPPPPPPXPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPXPPPPPPPPPPPPPPPPPPPPPPPPPPPPPPPPPPPPPPPPPPPPPPP |
| 76 | L | 0.97 | # LLLLLLLLLL-LLLVLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLVLLLLVLLLLLLLLLLLLLLLLLLLLLLLLLLLLLLLLLLLLLLLL |
| | | | LLLLVLLLLLLLLLLLLLLLLLLLLLLLLLLLLLLLLLLLLLLLLLLLLLLLLLLLLLLLLLLLLLLLLLLLLL |
| 77 | R | 0.967 | # RRRRRRRRRR-RRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRIRR$RRRRRRRRRRRRRRRRRRRRRRRRR |
| 78 | P | 0.994 | # PPPPPPPPPP-PPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPP |
| 79 | M | 0.964 | # MMMMMMMMMM-MMMMMMMMMMMMMMMMMMMMMMMMMMMMMMMMMMMMMMMMMMMMMMMMMMMMMMMMMMMMMMMM |
| | | | MMMMMMMMMMMMMMMMMMMMMMMMMMMMMMMMIMMMMNNMMMMNMMNMMMMNNMNMMMNNIMMMMMMMMMMMNMMMMMMMMMMMM |
| | | | MMNVMMMMMMMMMHMMMMMMMMMMMMMMMMMXMMNMNMMMMMMMMMMMMMMMHMMMMMMMMMMMMMMMMMMMMM |
| 80 | T | 0.928 | # TTTTTTTTTT-TTTTDTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTSTTTTTTTTTTTTTTTTTTTTTTITTTTTTTTITTTDTTTTNTTTTTTTTTTTTTTT |
| | | | TTTTTTTTTTTTTTTTTTTTTTXTTTTTTTTTTTTTTTTTTTTTT-TTTTTTDTTTTTTTTTTTTTTTTTTTTT |
| 81 | Y | 0.863 | # YFYYYYYYYY-YYYYYYYYYYYYWCYYYYYYYYYYYYYYYYYYFYFYYYYYFYYYYYYXYYYSYYYYYYYYYYYY |
| | | | YYYYYYYYRRYYYYYYYYYYYYYYYYYFYYYYYYYYYYYYYYYYYYYYYYYYYYYYYYYYYYYYYYYYYYYYYYYYYFYYYYYWY |
| | | | YYYYYYYYYYYYYYYYYYYYYYYYHYYYYYYCYYPFYYHYFYYYY-YYYYYYYYYYYYYYYYYYFFYYYYYYYF |
| 82 | K | 0.913 | # KKKKKKKKKK-KKKKRKKKKKKKKKKKKKKKKKKKKKKKKKKKRKKKKKKKKKKKKKKKKKKRKKKKKKKKKKKK |
| | | | KKKKKKKKRRKKKKKKKKKKKKKKKKKKKKKKKKKKKKQRKKKKKRRKKKKKKKKKKKKKKKKKKKKKKKKKKRKKKKKKKKKKK |
| | | | KKKKKKKKKKKKKKRRKKKQRKKKRKKKKQKRKKKKKKKKKKKKK-KKKKKKKKKKKKKKKKKKKKKKKKKKKK |
| 83 | A | 0.717 | # GGAGGAAAAA-GGSAAAGGGGGAAAGGGAAAAGGGAGGGGGAGAAAAAGAGAGGGAAAGGAGAAAGGAAAAGGGA |
| | | | AAAGGGAGAAAGVAAGGGGAAGAGGAGGAGGGAAGGGAAAAAGAGAAGGAGGGAGAAGGGGAGAGAAAAGAGAAGGGGGGAAAAG |
| | | | AAGGAAAGAAAGGGGGASGAAGAAAASAGAGAAGEAGGAGGGGGG-AAGGAGSAGGGGGAGGGGAAASAGGGGG |
| 84 | A | 0.961 | # AAAAAAAAAA-AAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAXAAAAAAAAAAAAAAAAAAAAAAAAAAAAA-AAAASASAAAAAAAAAAAAAAAAAAAAA |
| 85 | V | 0.588 | # VLVLLVVVVV-FLVVVLLLLIIVLVVLLVLLVLVLVLLVLLVFVLHVVRILLLLLLVVXLVLRVVLLMFVVLVPV |
| | | | VVVLLVVLIIVIVVVLVVLLVVLLLVVLLLMLLVFLLFLRFVLLLLVLVLLFVVFVVLVLLVLVVLVLVVVLVLILLLVLVVVLV |
| | | | VLLVVVVVVLVLLFLLLVVLRLLVIVVLLXVIVVLVVVWLLLLLV-VVLLVSVVVLLLVVLLLVHHVLVLLLLL |
| 86 | D | 0.946 | # DDDDDDDDDD-DDDDDDDDDDDDDGDDDDDDDDDDDDDDDDDDDDDDDDDDDDDDDDDDDDDDDDDDDDDDDDDD |
| | | | DDDDDDDDDDDDDDDDDDDDDDDDDDDDDDDNDDDDDDDDDDDDDDDDDDDDDDDDDDDDDDDDDDDDDDDDDDDDDDDDDDDDD |
| | | | DDDDDDDDDDDDDDDDDDDDDDDDDDDDDNDDDDDDDDDDDDDDD-DDDDDDDDDDDDDDDDDDDDDDDNDDDD |
| 87 | L | 0.907 | # LLILLILLLL-LLLLMLLLLLLLLLLLLLILLLLLLLLLLLILLILLLLLLLLLLLLLLLLLLLLLLILLLLLLL |
| | | | FLLLLLLLLLLLMLLLLLLLLLLLLLLLLLLLLLLLLLLFLLLLLLLLLLLLLLLLLLLLLLLLLLILLLLLLLLLLLLLLLLLL |
| | | | LLLLLLLLLILLLLLLLLLLLLLLLMLLLLLLLLLLLLLLLLLLL-LLLLLLLLLLLLLLLLLLLLLLLLLLLL |
| 88 | S | 0.964 | # SSSSSSSSSS-SSSSSSSSSSSSSSSSSSSSSSSSSSSSSSSNSSSSSSSSSNSSSSSSSSSSSSSSSSSSSSSS |
| | | | SSSSSSSSSSSSSSSSSSSSSSSSSSSSSSSSSSSSSSSSSSSSSSSSSSSSSSSSSSSTSSSSSSSSSSSSSSSSSSSSSSSSS |
| | | | SSSSSSSSSSSSSSSSSSSSSSSSSSSSSSSSSSSSSSSSSSSSS-SSSSSSSSSSSSSSSSSSSSSSSNSSSS |
| 89 | H | 0.941 | # HHHRHHHPHH-HHHHHHHHHHHHNHHHHHHHHHHHHHHHHHHHNHFHHWHHHHHHHHHHHHHHHHHHHFHHHHHH |
| | | | HHHHHHHHHHHHHHHHHHHHHHHHHHHHHHHHHHHHHHHHHHHHHHHHHHHHHHYHHHHHHHHHHHHHHHHHHHHHHHHHHHHHH |
| | | | HHHHHHHHHHHHHHHHHHHHHHHHHHXHHHHHHHHHHHHHHHHHH-HHHHHHHHHHHHHHHHHHHHHHHHHHHH |
| 90 | F | 0.989 | # FFFFFFFFFFXFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFPFFFFFFFFFPFFFFFFFFFFFFFFFFFFFFFFFFFFFFFFFFFFF |
| | | | FFFFFFFFFFFFFFFFFFFFFFFFFFFFFFFFFFFFFFFFFFFFF-FFFFFFFFFFFFFFFFFFFFFFFFFFFF |
| 91 | L | 0.975 | # LLLLLLLLLLLLLLLILLLLLLLLLLLLLLLLLLILLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLL-LLLLLLLLLLLLLLLLLLLLLLLLLLLL |
| 92 | K | 0.86 | # KRKRRKKKKKKKKKKKKKRKKKKKKKKKKKKKKRKKKKKKRKRKKKKKKRRKKKKKKKRKKKKKKKKKKKKKKKK |
| | | | KKKKKKKKRRKKRKKKRKRKKKEKKKKKKKKKKKKKKKKKKKKKKRRKKKKKKKKKKKKKKKKKKKKKKKKKKKKRKKKKKKKKK |
| | | | KKNKKKKKKKKRKKKKRKKKKKKKKKKKKKKKKKKKKKKKKKKKK-KKKKKRKKKKKRKKKKKKK-KKKKRRER |
| 93 | E | 0.932 | # EEEEEEEEEEEEEEEEEEEEEEEEEEEEEE$EEEEEEEEEEEEDEEEEEEEEEEEEEEEEEEEEEEEEEEEEEEE |
| | | | EEEEEEEEEEEEGEEEEEEEEEEEEEEEEEEEEEEEEEEEEEEQEKEEEEEEEEEEEEEEEEEEEEEEEEEEEEEEEEEEEEEEE |
| | | | EEEEEEEEEEEKEEEKEEEEEEEEEEEEEEEEEEEEEEEEEEEEE-EEEEEEEEEEEEEEEEEEE-EEEEEEEE |
| 94 | K | 0.884 | # KKKKKKKKKKKKKKKKKKKKKKKKKKKKKKKKKKKEKKKKKKKKKQKKKKKKKKEKKKKKKNKTKKKKKKKKKKK |
| | | | EKKKKKKKKKKKKKKKKKKKKKKKKKKKKKKKKQKKKKKKKEKKKEKKKKKKKKKKKKKKKMKKKQKKEKKKKKKKKKKKKKKKK |
| | | | KKKKKKKKKKQKKKKKKKKKKKKEKKKKKKKKKKKKKKKKKKKKK-KKQKKKKKRKKKKKKKKKK-KKKKKKKK |
| 95 | G | 0.979 | # GGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGEGGGGGGGGEGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGG-GGGGGGGGGGGGGGGGGGG-GGGGGGGG |
| 96 | G | 0.978 | # GGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGVGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGG-GGGGGGGGGGGGGGGGGGG-GGGGGGGG |
| 97 | L | 0.975 | # LLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLLLLLLLLLLLLLPL |
| | | | LLLLLLLLLLLLLLLPLLLLLLLLLLLLLLLLLLLLLLLLLLLLL-LLLLLLLLLLLLLLLLLLL-LLLLLLLL |
| 98 | E | 0.874 | # EEEEEEEEEDEEEEEDEEEEEEEEDEEEEEEEEDEEDEDEEEEEEEEEEEEEEEEEEEEEEEDEEEDEEEEEEEE |
| | | | EEEEDEEEEEEEEEEEEEDEEEEEDEEEEEEEEEEEEEEEEEEEEEEEEEEEEEEEEEEEEEDEDEEDEEEEEEEEEEEEEEEEE |
| | | | EEDEEEEEEEDEDEEEEEEEDEEEEEEEEEEEEEDEEDDEDEEEE-EGEEEEEEEEEEEEEEEED-EEEEEEEE |
| 99 | G | 0.959 | # GGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGG-GGGGGGGGGGGGGGGGGGG-GGGGGGGG |
| 100 | L | 0.899 | # LLLLLLLLLLLLLLLMLLLLLLLLLLLLLLLLLLLLLLLLLLLLLLLLLLLLLLLLLLLILLMLLLLLLLLLLLL |
| | | | LLLLLLLLLLLLLLLLLLLLLLLLLLLLLLLLLLLLLLLLLLLLLLLLLPLLLLLLLLLLLMLIIILLLLLLLLIIILLLLLLLL |
| | | | LLLILLLLLILILLLLLLLLLLLLLLXLLLLLLLLLLLMLLLLLL-LLLLLLLLLLLLLLLLILL-LLLLLLLL |
| 101 | I | 0.771 | # VVIIIIIVIIIIIIIIITTIIVIVIIIIIIIIIIIIIIIVIIIVIIIIIVIIIVIVIIVIIVIIVIIIIIIVVII |
| | | | IIIIIIIIVVIIIIIIIIIIIIIIIIIIVIIIVIIIIIVIIIIITIIIIIIIIIIIIITVIIIVIIIVIVIVIIVVIIIVIVIII |
| | | | IIIIVVTIIIIIIIIIIVIIIIVVIIIIIIIVVIIVIIIVIIVIV-VVIIVVVVIIVIIIIVVVI-VVVVVIIV |
| 102 | Y | 0.706 | # HYYHHYHHHYYYHHYYYHHYHHYYYYYHYHYYYYHFYHYYHYYWYYYYYYYYHYHYYYYYWYYFHYYYYHHYYYY |
| | | | YHYYYYYWHHHYHHHYYYYHHYYYYHYCWYHWYYYYYYHYHHYHWHYYYWHHYHYHYYYWYHYYYYYHYYYYYYYYYYYYYYHYY |
| | | | HYYYHHYYHYYYYHYYHYYHYYYYYYHYYHHWHYWWYYYYYYHYH-YHYYYYYYHYYHHHYYYHY-VVVFWHYH |
| 103 | S | 0.966 | # SSSSSSSSTSSSSSSSPSSSSSSSSSSSSSSSSSSSSSSSSSSSSSSSSSSSSSSSSSSSSSSSSSSSSSSSSSS |
| | | | SSSSSSSSSSSSSSSSSSSSFSSSSSSSSSSSSSSSSSSSSSSSSSSSSSSSSSSSSSSSSSSSSSSSSSSSSSSSSSSSSSSSS |
| | | | ASSSSSSSSSSSSSSSSSSSSSSSSSSSSSSSSSSSSSSSSSSSS-SSSSSSSSSSSSSSSSSSS-SSSSSSSS |
| 104 | Q | 0.878 | # QQRQQQQQQQQQQQQQPQQQQQQQQQQQQQKQQQQQQQQQQRQQQQQQQQQQQQQQKQQQQQQQQPQQQHQQQQQ |
| | | | QQHQQQQQQQQQQQQQQQQQQQPQPQQQQQQQQQQQQRQQQQQLQQQLQQQQRQQQHQQQPQQQQQQQQQQQQQQRQRQQQQQRQ |
| | | | QQQQQQQQQQPQQQQQQQQQQQQQQQQQQQQQQQQQQQQQQQQQQ-QQQQQQQQQQQQQQQQQQQ-QQQQQQQQ |
| 105 | K | 0.71 | # RKKKKKKKKRRKKKQKKKKQRQRKRKKKKRRKKKKRKRRKRKKKRREKNRRKKKRQRRXKKRKKKKRRKKKKKKK |
| | | | KKKRKRKKKKRRKQGKKKRKQKKRKKKKQKKKKQRKKKQKRKKKKKKKKRKKKKKKKRQKKQKKKQRKRKRQRRKKKKKRQQKKK |
| | | | RKKKKKKKQRKKKKKKKKQRRKQRKKKQRKRKRKKKKKKKKKRRQ-KKKKKKKKKKKRKKKKQRK-KKKKKKKR |
| 106 | R | 0.962 | # RRRRRRRRRRRRRRRRRRRRRRRRRRRRGRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRRRRRRRRRRRRGRRRRRRRRRRRRRRRRRRRRRRRRRRRRRRRRRR |
| | | | RRRRRRRRRRRRRRRRRRRRRRRRRRRRRRRRRRRRRRRRRRRRR-RRRRSRRRRRRRRRRRRRR-RRRRRRRR |
| 107 | Q | 0.903 | # QQQQQQQQQQQQQQRQQQQQQQQRQQQQQQQQQQQQQQRQQQQQQQQQQVQQQQQKQQQQQQQQQQQQQQQQQQQ |
| | | | QQQQQQQQQQQQQQQQKKQQQQKQQQRQQQQQQQQQQQQQQQQQQQQQQQQQQQQQQQQQQQQQQQQQQQQQQQQQQQQRQRQQQ |
| | | | QQRQQQQQQQQQQQQQQQQQQQQQQQQKQQQQQQQQQQQQQQQQQ-QQRQQQQQQQQRQQQQQQQ-QQQQQQQQ |
| 108 | D | 0.837 | # DDEDDDEDDDDDDDDDEDDDDDDDDEDDDDDDDEDDDDDDDEDDDDDDDDDDDEDDDDDDDEEEDEDDDDDDDDD |
| | | | DDDDDDDDEEDDDDDEDDDEDDEGEDEEEDDDDEDDDEDDDEDEDDDDDDDDEDDDDDDEEDDDDDEDDDDDDEDEEEDDDDDED |
| | | | DEDDDDDDDDEDDDDDDDDDDDDDEDXDEDDDDDDDDEDDEDDDD-DDDDDDDDDDDDDDDDDDD-DDDDDDDD |
| 109 | I | 0.979 | # IIIIIIIIIMIIIIIIIIIIIIIIMIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIIIIIIIIIIIIIIIIIIIIIIIIIIIIIIIIIIIIIIIIIII |
| | | | IIIIIIIIIIIIIIIIIIIIIIIIIIIIIIIIIIIIIIIIIIIII-IIIIIIIIIIIIIIIIIII-IIIIIIII |
| 110 | L | 0.981 | # LLLLLLLLLXLLLLLLLLLLLLLLXLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLXLLLLLLLLLLLLLLLLLLL-LLLLLLLL |
| 111 | D | 0.959 | # DDDDDDDDD-DDDDDDDDDDDDDD-DDDDDDDDDDDDDYDDDDDDDDDDNDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDXDDDDDDDDDDDDDDDDDDD-DDDDDDDD |
| 112 | L | 0.955 | # LLLLLLLLL-LLLLLLLLLLLLLL-LLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLQLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLL-LLLLLLLL |
| 113 | W | 0.959 | # WWWWWWWWW-WWWWWWWWWWWWWW-WWWWWWWWWWWWWWWWWWWWWWWWWWWWWWWWWWWWWWWWWWWWWWWWWW |
| | | | WWWWWWWWWWWWWWWSWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWW-WWWWWWWW |
| 114 | V | 0.786 | # VVIVIIIVI-IVVVVLVVVVVVII-VIVVIIVVVVIIIIIVIVVIIVIIVVVVVIVIIVVVVVIVVVIVVVVIVI |
| | | | VIVVVVVVVVIIVVIVVVIVVVVVVVVIVVVVVVVVVVVVVFVIVVVVVVVVVVVVVVVIVIVVIVIVVVIVIVVVVMVVVVIVV |
| | | | IVVVVVIIVIVVVVVVVVVVVVVVVVVVVVVIVVVVIVIVVVVVVIIVVVIVIVVVVVVVVVVVV-VVVVIIVI |
| 115 | Y | 0.943 | # YYYYYYYYY-YYYYYYYYYYYYYY-YYYYYYYYYYYYYYYYYYYYYYYYYYYYYYHYYYYYYYYYYYYYYYYYYY |
| | | | YYYYYYYHYYYYYYYYYYYYYYYYYYYYYYYYYYYYYYYYYHYYYYYYYYYYYYYYYYYYYYYYYYYYHYYYYYYYYYYYYYYYY |
| | | | YYYYYYYYYYYYYYYYYYYYYYYYYYYYYYHYYYHYYYYYYYYYYYYYYYYYYYYYYYYHYVYVY-YYYYYYYY |
| 116 | H | 0.854 | # NHHHHHMHH-HHHHNHHHHNNNHH-HHHHHHHNHHHHHHNNHHHHHDHHHHHHHHNHHNHHHHHHHHHHHHHNHH |
| | | | HHHHHHHHHHHHHHHHNHHHHHHHHHNHNHNHNHHHHHHHHHHNHHHHNHHNHHHHHNHHHHHHHHHHHHHHHHHHHHHHHHHHH |
| | | | HHHHHHHHNHHHHHHNHHNHHHNHNHHHHHHHNHHHHHHNHHHHHHHHNMHHHHHHTHHHNHHHH-HHHNHHHH |
| 117 | T | 0.969 | # TTTTTTTTT-TTTTTTTTTTTTTT-TTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTHTTTTTTTT-TTTTTTTT |
| 118 | Q | 0.95 | # QQQQQQQQQ-QQQQQEQQQXQQQQ-QQQQQQQQQQQQQQQQQQQQQQQQQQQQQQQQQQQQQQQQQQQQQQQQQQ |
| | | | QQQQQQQQQQQQQQQQQQQQQQQQQQQQQQQQQQQQQQQQQQQPQQQQQQQQQQQQQEQQQQQQQQQQQQQQQQQQQQQQQQQQQ |
| | | | QQQQQQQQQQQQQQQQQQQQEQQQQQQQQQQQQQQQQQQQQQQQQQQQQQQQQQQQKQQQQQQQQ-QQQQQQQQ |
| 119 | G | 0.966 | # GGGGGGGGG-GGGGGGGGGGGGGG-GGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGDGGGGGGGGGGGGG |
| | | | GGGGGGGGGGGGXGGAGGGGGGGGGGGGGGGGGGGGGGGGGGGGGGGGGGGGGGGGGGGGGGGGG-GGGGGGGG |
| 120 | Y | 0.864 | # YYYYYYYYYYYYYYYYYYYYYYYFYYYFYYYFYYYYYYYYYYYYYYYYYFYYYYYYYYYYYYYYYYYFYYYYYYY |
| | | | YYFYYYYYYYYYYFFYYYYYYYFYFYYYFYYYYYYYYYYFFYYYYYYYYYYYYYYFYYYYYYYYYYYYFYYYYYYYYYYYYYFYY |
| | | | YYYYYYYYYYYYFYYSYFYYYYFYYYYYYYYYYYYYYFYYYYYYYYFYYYYYFYYYFYYYYYYYF-FFYYYYYY |
| 121 | F | 0.958 | # FFFFFFFFFFFFFFFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFLFFFFFFFFFFFFFFFFFFFFFFFFFFFFFFFFFFFFFFFPFFFFFFFFFFF |
| | | | FFFFFFFFFFFFFFFSFFFFFFFFFFFFFFFFFFFFFFFFFFFFFFFLFFFFFFFFFFFLFFPFF-FFFFFFFF |
| 122 | P | 0.994 | # PPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPP-PPPPPPPP |
| 123 | D | 0.982 | # DDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDDDDDDDDDEDDDDD |
| | | | DDDDDDDDDDDDDDDDDDDDDDDDDDDNDDDDDDDDDDDDDDDDDDDDDDDDDDDDDDDDDDDDD-DDDDDDDD |
| 124 | W | 0.97 | # WWWWWWWWWWWWWWWWWWWWWWWWWWWWWWWWWWWWWWWWWWWWWWWWWWWWWRWWWWWWWWWWWWWWWWWWWWW |
| | | | WWWWWWWWWW$WWWWWWWWWWWWWWWWWWWWWWWWWWWWWWWWWWWWWWWWWWWWWWWWWWWWWWWWWWWWWWWWWWWWWWWWWW |
| | | | $WWWWWWWWWWWWWWWWWWWWWWWWWWXWWWWWWWWWWWWWWWWWWWWWWWWWWWWWWWWWWWWW-WWWWWWWW |
| 125 | Q | 0.9 | # QQQQQQQQQQQQQQQQQQQQQQQQQQQQQQQQQQQQQQQQQHHQQQQQQHQQQQQQQQQQQQQQQQQQQHQQQQQ |
| | | | QQQQQQQQQQQQQQQQQQQQQQKQQQQQQQQQQQQQQQQQQHQQQQQQQQQQQQQQQQQQQQQQQQQQQQQQQQQQQQQQQQQDQ |
| | | | QQHQQQQQQQQQQQQQQQQQQQQQQQQQQQQQQQQQQQQQQQQQQQQQQQQQQQQQQQQQQQQQQ-HQQQQQQQ |
| 126 | N | 0.909 | # NNNNNNNNNNNNNNNNNNNNNNNNNNNNNNNNNNNNNNNNNNNNNNNNNNNNNSNCNNNNNNNNNNNNNNNNNNN |
| | | | NNGNNNNNNNNNNNNNNSNNNNNNNNNNNNNNNNNNNNNKNNNNNNNNNNNNNNNGNNNNNNNNNNNNNNNNNNNNNNNNNNNNN |
| | | | NNNNNNKNNNNNNNNNNNNCNNNNXNNNNNNNNNNNNNNNNNNNKNNGNNNNNNNNNNNGNNNNN-NNNNNNNN |
| 127 | Y | 0.973 | # YYYYYYYYYY$YYYYYYYYYYYYYYYYYYYYYYYYYYYYYYYYYYYYVYYYYYYYYYYYYYYYYYYYYYYYYYYY |
| | | | YYYYYYYYYYYYYYYYYYYYYYYYYYYYYYYYYYYYYYYYYYYYYYYYYYYYYYYYYYYYYYYYYYYYYYYYYYYYYYYYYYYYY |
| | | | YYYYYYDYYYYYYYYYYYYYYYYYYYYYYYYYYYYYYYYYYYYYYYYYYYYYYYYYYYYYYYYYY-YYYYY-YY |
| 128 | T | 0.979 | # TTTTTTTTTT-TTTTTTTTTTTTTTTTTTTTTTTTTTTTTTTTTTTTTTTTTTTTTTTTTTTTTTTTTTTTTTTT |
| | | | TTTTTTTTTTTTTTTTTTTTTTTTTTTTTTTTTTTTTTTTTTTTTTTTTTTTTTTTTTTTTTTTTATTTTTTTTTTTTTTTTTTT |
| | | | TTTTTTTTTTTTTTTTTTTTTTTTTTTTTTTTTTTTTTTTTTTTTTTTTTTTTTTTTTTTTTTTT-TTTTT-TT |
| 129 | P | 0.892 | # PRPPPPPPPP-PPPPPPPPPPPPPPPPPPPPPPPPPPPPPPPPPPPPPPPPPPPPPPPPPPPPPPPPPPPPPPPP |
| | | | PP--P-PPPPPPPPPPPPPPPPPPPPPPPPPPPPPPPPKPPPPPPPPPPPPPPPPPPPPPPPPPPPPKPPPPPPPPPPPPKQPPP |
| | | | PPPPPSPPPPPPPPPPSPPPPPPPSPPPPPPPPPPPAEPQPPSPPPPPPPPPQPPPPPPPPPPPP-PPPPP-PP |
| 130 | G | 0.942 | # GGGGGRGGGG-GGGGGGGGGGGGGGGGGGGGGGGGGGGGGGGGGGGGGGGGGGGGGGGGGGGGGGGGGGGGGGGG |
| | | | GG--G-GGGGGGGGGGGGGGGGGGGGGGGGGGGGGGRGGGGGGGGGGGGGGGGGGGGGGGGGGGGGGGGGGGRGGGGGGGGGGGG |
| | | | GGGGGGGGGGGGGGGGGGGGGGGGGGGGGGGGGGGGGGGGGGGGGGGGGGGGGGGGGGGGGGGGG-GGGGG-GG |
| 131 | P | 0.975 | # PPPPPPPPPP-PPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPP-PPPPP-PP |
| 132 | G | 0.971 | # GGGGGGGGGG-GGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGG-GGGGG-GG |
| 133 | I | 0.614 | # TITIVVTPPI-TITIVVIIITTTTIIIIITTIIIIIVIIIIVIPITTIVTTTTTIIIIVVVITITVIVIITTTVI |
| | | | IIVITTIIIIVVIITITTVITITITVITTIIVVIVTVITTVIIIIVVTIITIITVVIIITIIVTVTTIVTIITTVVIVVITTITV |
| | | | VTVVVVVTVITITVIIIIIITVXTITTPTIITIIITVTVTTIITITIIITTITTIVIVIVITITT-ITIIT-TV |
| 134 | R | 0.968 | # RRRRRRRRRR-RRRRRRRRRRRRRRRRRRRRRRRRRRRRRRRRRRRRRRRRRRRRRRRRRRRRRRRRRRRRRRRR |
| | | | RRRRRRRRRRRRRRRRRRRRRRRRRRRRRRRRRRRRRRRTRRRRRRRRRRRRRRRRRRRRRRRRRRRRRRRRKRRRRRRRRRRRR |
| | | | RRRRRRRRRRRRRRRRRRRRRRRRRRRRRRRRRRRRRRRRRRRRRRRRRRRRRRRRRRRRRRRRR-RRRRR-RR |
| 135 | Y | 0.738 | # YYYYYYYFFF-YYFFYYYYYFFYFFYYYYYYYYYYYFYYYYYYFYYWYYWFFFFYYYYWYFYYYFYYYYYFFYYY |
| | | | YYFYFFYYWWYFYYYYFCYFFYYYYFYFFYYHYFYFFYFYYYYYYWYYYYYYYFYYYYYYYYFFYFYYYYYYFFYYYYYYFFYYY |
| | | | YYYYYFYYYYFYFYYYYYYYYYXYYYYFFYYYYYYFFFFFFYYFFFYYYFFYFFYFYYYYYFYFY-YFYYF-FY |
| 136 | P | 0.985 | # PPPPPPPPPP-PPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPP-PPPPP-PP |
| 137 | L | 0.947 | # LLLLLLLLLL-LLLLLLLLLLLLLLLLLLLRLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLILLLLILLLLLLLLLLLLLLLLL |
| | | | LLLLLLLLLLLLLLLLLLLLLLLLXLLLLLLLLLLLLLLLLLLLLLLILLLLLLLLLLLLLLLLI-LLLLL-LL |
| 138 | T | 0.88 | # TATTTTTTTT-TTTTTTTTTTTTTTTTTTTTTTTTTTTTTTTCTTTTTTTTTTTTTTTTTTTTTTTTCTTTTTTT |
| | | | TTTTTTTTTTTCTCCTTCTTTTTTTTTTTCTTTTTTTTTTTTTTTTTTTTTTTTTTTTTTTTTTTCTTTTTTTTTTTTTTTTCCT |
| | | | TTTCTTTTTTTTTTTTTTTTTCTTTTTTTTTTTTTCCTCTTTTCTTTTTTTTTTTTTRTTTTTTT-TTTTT-TT |
| 139 | F | 0.887 | # FFFFFFFFFF-FFFFFFFFFFFFFFFFFFFFFFLFFFFFFFFFFFFFFFFFFFFFFLFFFFFLFFFFFFFFFFFF |
| | | | FFFFFFFFLPFLFFFFFLFXFFLFFFFFFFFFFSFFFFYFFFFFFFFFFFFFFFFFYFFFFFFFLFFFFFLFFFFFFFFFFFFFF |
| | | | FFFFFFFLFFFFFFFFFFFYYFFFLYFFFFFFFFFFFFFFFLFFFFFFFFFFFFFFFFFFFFFFF-FFFFF-FF |
| 140 | G | 0.949 | # GGGGGRGGGG-GGGGGGGGGGGGGGGGGGGGGGGGGGGGGGGGGGGGGGGGGGGGGGGGGGGGGGGGGGGGGGGG |
| | | | GGGGGGGGGGGGGGGGGGGXGGGGGGGGGGGGGGGGGGGGGGGGGGGGGGGGGGGGGGGGGGGGGGGGGGGGGGGGGRGGGGGGG |
| | | | GGGGGGGGGGGGGGGGGGGGEGGGGGGGGGGGGGGGGGGGGGGGGGGGGGGGGGGGGGGGGGGGG-GGGGG-GG |
| 141 | W | 0.977 | # WWWWWWWWWW-WWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWW-WWWWW-WW |
| 142 | C | 0.963 | # CCCCCCCCCC-CCCCCCCCCCCCCCCCCCCCCCCCCCCCCCCCCCCCCCCCCCCCCCCCCCCCCCCCCCCCCCCC |
| | | | CCCCCCCCCCCCCCCCCCCCCCCCCCCCCCCCCCCCCCCCCCCCCCCCCCCCCCCCCCCCCCCCCCCCCCCCCCCCCCCCCCCCC |
| | | | CRCCCCCCCCCCCCCCCCCCCCCCCCCWCSCCCCCCCCCCCCCCCCCCCCCCCCCCCCCCCCCCCXCCCCC-CC |
| 143 | F | 0.903 | # FFFFFFFFFF-FFFFFFFFFFWFFFFFFFFFFFFFFFFFFFFFFFFFFFFFFFFFFFFFFFFFFFFFFFFFFFFF |
| | | | YFFFFFFFFFYFFFFFFFYFFFFFFFFFFFFFFFFFFFFFFFFFFFFFFFFFFFFFFFFFFPFFYFYFFFFFFFFFFFFFFFFFF |
| | | | YFFFFFFFFFFFFFFFFFFFFFFFFFFCFFFFYFFFFYFFFFFFFFFFFFFLFFFFFFFFFFFFFXFFFFF-FF |
| 144 | K | 0.969 | # KKKKKKKKKK-KKKKKKKKKKKKKKKKKKKKKKKKKKKKKKKKKKKKKKKKKKKKKKKKKKKKKKKKKKKKKKKK |
| | | | KKKKKKKKKKKKKKKKKKRKKKKKRKKKKKKKKKKKKKKKKKKKKKKKKKKKKKKKKKKKKKKKKKKKKKKKKKKKKKKKKKKKK |
| | | | KKKKKKKKKKKKKKKKKKKKKKKKKKKKKKKKKKKKKKKKKKKKKKKKKKKKKKKKKKKKKKKKKKKKKKK-KK |
| 145 | L | 0.989 | # LLLLLLLLLL-LLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLL-LL |
| 146 | V | 0.963 | # VVVVVVVVVV-VVVVVVVVVVVVVVVVVVVVVVVVVVVVVVVVVVVVVVVVVVVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVVVVVVVVVVVVVVV |
| | | | VVAVVVVVVVVVVVVVVVVVVVVVVVVVVVVEVVVVVVVVVVVVVVVVVVVVVVVVVVVVVVVVVVVVVVV-VV |
| 147 | P | 0.984 | # PPPPPPPPPP-PPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPP-PP |
| 148 | V | 0.909 | # VVVVVVVVVV-VVVVVVVVVVVVMVVVVVVVVVLVVVVVVVVVVVVVVVLVVVVVVVVVVVVVVVVVVVVVVVVV |
| | | | LVVVVVVVVVVVVVVVVVVVVVVVVVVVVMVVVVVVVMVVVVVVVVVVVVVVVVVVVVVVVVVVVVLVVVVVVVVVMVVVVMVVV |
| | | | VVVVVVVVAVVVVVVVVVVVVVVVVVVLVVVVVVLVVVVVIVVVVVVVVVVVVVVVVVVVVVVVVVVVVVV-VV |
| 149 | E | 0.755 | # EDEEDDEDDE-EEEEDEDEDEEEEEEEEEEEEEEEEEEEEEEEEEEEEEDDDDDEDEEEEXDEGKEDEDEKDDDE |
| | | | EDEDEEDEEEESESEEEEEPEEEEEKEDEDEDEEDEEEEEEEEEEEDEEEEDEDEEEEEEDEDEEEDEEDDEEDDEEEEEEEEDD |
| | | | EEDEEEEEEEEDEEEENEEEEEDDEEEEEDEEEEEEEEEEEDEEEEEEEDEEEEDEEGDEEEDEEEDEEEE-EE |
| 150 | P | 0.848 | # PPQPPPPPPP-PPPPPPPPPPPPRPPRPQPPPPPPPPPPPPPPPPPPPPPPPPPPPPPXPPPPPPPPPPPPPPPR |
| | | | QPPPPPPPPPPPPPPPPPPAPPPAPPQPPPPQTPPPPPPPSPPPPPPPPPPPSPPPPPPPPPPPPPPPPPPPPPPPPPPPPPPPP |
| | | | PPPPPPPTPPPPPPPPPPPPPXPPPTPKQPPPPPPPPPPPPPPPPPPKPPPPPPPPPPPKPPPPPPPPPPP-PP |
| 151 | E | 0.736 | # EEEEDEGEEE-DEEEE-DDEEDEEEEEEEEDEEEEEEDEEEEEDEDEEEDEEKEDEDQEXEEDDEDEGDEEDDEE |
| | | | EEDGDEEEEEDEEEEEEEEEEEEEE-EDEDEEEEAEEDDEEEDEEEEEDDEEEDEEEDEDDDEEEEDEEEDGEEDEDEEEEEDDE |
| | | | DEDEGDEEGEEEDEEEEEEDEXDEEEEEEDEEEEEEEDEEEEEEEEEEEDEEEEEEDEEEEEEEEEDEEDE-EE |
| 152 | K | 0.666 | # KQQKQK-KKK-QKK-K-KKQKKKKKKQQQK-KKQKKK-KKK-QKKQKKKQQQQQKKKKKEKQKEQ-KRKKEKKKK |
| | | | KKKQKQKKQQKKKKKKQQKQQKKEKNKQQQKQKKKKKQKKEKEQKKKKEKKQKKKKKKKKQKKKKKEQKKQQKKKEKQQKKKKQK |
| | | | KKQKKKKKQQEKK-KKEKKKKKKKKKKQKKKKKKQKQKEQLKQKKRKKQKKEKKKKKNKKKKKKKKKKKKK-KK |
| 153 | V | 0.747 | # VVVIVV-VVV-VVV-V-VVVVVVIVVVVVI-VVVVVI-IVV-VVVVIIIVVVVVVVVVVVXVVVV-VVVIIVVVI |
| | | | VVVVVVIVVVIVVVIVVVIVVIVVVEVVVVIVVIVVVVVVVIVIVIVIVVIIVVIVVVIVVVIIVVIIIVVVIVVVVVIIVIVVI |
| | | | VVVVVVIVVVVVV-VVVIVVVVVIVVVVVVVVVIIVVVVVVIVVIVIVVVIVVIIVIVIVIVIIVVVVVVV-VI |
| 154 | E | 0.918 | # EEEEEE-EEE-EEE-E-EEEEEEEEEEEEE-EEEEEEEEEE-EEEEEEEEEEEEEEEEEEEEEEE-EEEEEEEEE |
| | | | DEEEEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEE-EEEEEEEEEEEEEEEEEEEEEEEEEEEEEEEEEEEEEEEEEEEHEEEEEEEEEEEEE-EE |
| 155 | E | 0.779 | # EEEEKK-EEEEKEE-KEEEKEEEEEEEEEE-EKEEEARKKE-EEEKEEEREEEEEEEEEKEKE-E-KEEEEEEEE |
| | | | EEEEEEAEEEEEEEEEEEEEEKEKEEEKEEEEEEEEEEEEAEKKEEKKEEEKEEEEEEEEKEEEEEKEEEKEEEEKEKKKEEEKE |
| | | | EEEEEEEEEEKKE-EEKEEEEXEEEEEEEKEEEEEKEEKEEEEEEEEEEEEKEEEEEEEEEEEEEEEEEEE-EE |
| 156 | A | 0.855 | # AAAAAA-AAAAAAA-DAAAAAAAAAAAAAA-EAAAAAEAAAAAAAAAAAAAAAAAAAAAAAAE-A-AAAAAAAAA |
| | | | AAAAAAAAAAAAAAAAAAAAAAAAA-AAAAADAAAAAAAAAAAAAAAAAAAAAAAAAAAAAAAAAAAAAVAAAAAAAAAAAAAAA |
| | | | AAAAVAAAAAAYA-AAAAAAAAAAAAAAAAAAAAAAAAAAAAAAAAVAAAAAAAAAAAAAAAAAAAAAAAV-AV |
| 157 | W | 0.744 | # NNTNNN-NNNNNNNNNNNNNNNNNNNNNNN-NNNNNNNNNNSNTNNNNNNNNNTTNNNNNNNN-N-NNNNNNNNN |
| | | | TNNNNNNNNNNNNNNNNNNNNNNNNNNNNNNNNNNTNNNNNNTNNHNNNNNNNNTNNTNNNSNTNSNNNTNNNNNNTNTNTNNNT |
| | | | NNTTNNNNTNNNNNNNNTTNNNNTNNNNNNNNNTTTNNNNTNNNNTTNNTTNNTTTTTTNNTTTTTTTTTT-TT |
| 158 | E | 0.598 | # EEATEEEEEEEEEEEEEEEEEEGEEEEEEREVEKEEEEEEEEQEEEAEKEEEEEGKAEEEEEK-EEEEEEKEEEE |
| | | | EEEEEEEEEEKEEENEEEEEEEEEEETEEEEASEEEEEEEEKEEEEEEKEEEEQKEEIAEEAEAEEEEEEEEKKEEEEEEEEEEE |
| | | | KAEEEKKEEKEEEEEEDVEEEEEEEEEEEEAEEEEEEEEEAKEKEEVEKAAEEAVAVVVEKVAVVVVVAVE-VE |
| 159 | G | 0.89 | # GGGGGDSGGGGGGGGGEGGGGGGGGGGGGGEGGGGGGGGGGRGGGGGGGGGGGGGGGGGGGGG-GERGGGAGGGG |
| | | | GGGGGGGGGGGGGGGGGGGGGGGGGGGGGGGGGGGGGGGGGGGGGGGGGGGGGGGGGGRGGGGGGGGGGGGGGGGGGGGGGGGGG |
| | | | GGGGGGGGGRGGGGGGGGGGGXGGGGGGGGGGGGGRGGGGGGGGGGGGGGGGGGGGGGGRGRGGGGGGGGG-RG |
| 160 | E | 0.972 | # EEEEEEEEEEEEEEEEEEEEEEEEEEEEEEEEEEEEEEEEEEEEEEEEEEEEEEEEEEEEEEE-EEEEEEEEEEE |
| | | | EEEEEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEE-EE |
| 161 | N | 0.863 | # NNNNNNNNNNNNNNNNNNNNNNNNNNNNNNNNNKSNNNNNNNNNNNNNNNNNNNNKNNNRNNNNNNNNNNNNNNN |
| | | | NNNNNNNNNNNDNDNNNNNNNNNNNNNNNNNNNNNNNNNNTNNNNNNNNNNNNNNNNNNNNNNNNNNNNNNNNNNNNTNNNNNNN |
| | | | NNDDNNNNNNNNKNNNNNNNNNNNNNNNNNNNNDNNNNNNNNNNNNNNNNKNNDNNNDNNNDNNNNNNNNNNDN |
| 162 | N | 0.829 | # NNTNNNSKNNNINNNNSNNNNNNNNNNNNSSNNNNNKNNNNDNNNINNNNNTNNTSNNNNXNISNSSNNNSNNNS |
| | | | NNNNNNNNNNTNNNNNNDNNNNNNNNNNNNNNNNKTNNNNNNNNNNNNNNNNNNTNNNNNNNNNNNNNNNNNNNNNNNNNNNNNN |
| | | | TNNNNTNNNANNNNNNNNNNNNNNNNNNNNNNNNNTNNNNNNNINNNNNNNNNNNNNNNTNNNNNNNNNNNNNN |
| 163 | S | 0.613 | # KSSCSSSSSSSSNSSSSSCCSSSSSSSCCSSTSCRSSSSSCSSSSSCSSSCSSCSSCRKCSCCSSSYSCCCCSSY |
| | | | CSCSCVSRSSSSCSCCSTSSCSCSCSCCNSSNRKSKSSCSSCCSTSSSSSTSCCCSSSSSSCKCSCTSSCSSSSCCCCCSCSSCS |
| | | | SCSICSSCCSCCCSSSSCCSSIRCXSCCSCCSCCSSSCSKCSSSRCCSCCCSSCCCCSCSCCCCCCSCCCCCCS |
| 164 | L | 0.964 | # LLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLLLWLLLWLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLXLLLLLLLLLLLLLLLLLLLLLLLLLLLLLLLLLLLLLLLLLLLLLLLLLL |
| 165 | L | 0.982 | # LLLLLLLLLLSLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLVLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLLLL |
| 166 | H | 0.966 | # HHHHHHHHHHHHHHHHHHHHHHHHHHHHHHHHHHHHHHHHHHHHHHHHHHHHHHHSHHHHHHRHHHHHHHHHHHH |
| | | | HHHHHHHHHHHHHHHHHHHHHHHHHHHHHHHHHDHHHHHHHHHHHHHHHHHHHHHHHHHHHHHHHHHHHHHHHHHHHHHHHHHHH |
| | | | HHHHHHHHHHHHHHHHHHHHHHAHHHHHHHHHHHHHHHHHHHHHHHHHHHHHHHHHHHHHHHHHHHHHHHHHHH |
| 167 | P | 0.978 | # PPPPPPPPPPTPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPP |
| 168 | M | 0.676 | # VMMMMIMLIM-MMIMMMMMMMMMLMMMMMMAMIMMMMMMMMVIVMMMMMMMMMITMKIMXIMMAMAIMIIMIMMI |
| | | | ILMMMMIVMMVMMMIMMMMIMIMMNMMMMIMMMIMIMVMMMMMMMMMMIMMTMMMMMMMIMMMMMMMMMMMMMMMMLMMMMMMMM |
| | | | VMILMVMMVMMMMMMMMMMMMIMMLMMMMMMVMMIVMIMIIMMMMIIMMMAMLMMAAMMMMIIMMIMMIAVIIM |
| 169 | S | 0.73 | # SSSHSSNSSS-SSSSSSSSSSSSSSSSSSSSSSSSSSSSSSSSCSSSSSSSSSSSSSSSSSSSSSSSSSSSSSSS |
| | | | NSSSSSSSSSSSSSSSGSSCSSSSSSSSSSSSSSSSSSSSSSNSSSSSSSSSSSSSSSSSSHSNSNASSNSSSSSSNSNSNSSSN |
| | | | SSSNSSSSSSSSSSSSSNNSSSSNSSSSSSSSTSNSSSSSNSSSANNSSNNSSTNNNNNSSNNNNNNNNNNNNN |
| 170 | L | 0.748 | # LLLQQLQQQQ-LLQLLLQQQLQLQQQLQQLLLLQLQLLQLLLQQLLQLLQQQQQQLLLLLLQLLLLQLLLLLLLL |
| | | | QQQQLLLLQLLLQLQLLLLQQLLLLLQQQLLLLLLLLLQLQQQLLLQLLQLLLQQLLQQLQQLQLLQQTQQLQQQLQLQLQLLQT |
| | | | LQLQQLLLQQQQLLLLQQQLLLLQLLQLLLQLQQQLLTQLQLLQLLLLQQLQLLQLLLQLQLQLLLLLLMLLLL |
| 171 | H | 0.972 | # HHHHHHHHHH-HHHHHHHHHHHHHHHHHHHHHHHHHHHHHQHHHHHHHHHHHHHHHHHHHHHHHHHHHHHHHHHH |
| | | | HHHHHHHHHHHHHHHHHHHHHHHHHHHHHHHHHHHHHHHHHHHHHHHHHHHHHHHHHHHHHHHHHHHHHHHHHHHHHHHHHHHHH |
| | | | HHHHHHHHHHHHXHHHHHHHHHHHHHHHHHHHHHHHHHHHHHHHHHHHDHHHHHHHHHHHHHHHHHHHNHHHHH |
| 172 | G | 0.963 | # GGGGGRGGGG-GGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGGGGGGGGGGGGGGGGGGGGGGGGGGGG |
| | | | GGGGGGGGGGGGGGGGGGGGGGGGGGGGGGGGGGGGGGGGGGGGGGGGGGGGGGGGGGG6GGGGGGGGGGGGGG |
| 173 | M | 0.804 | # MMMMVITMMM-DMMMMMMMMIMVMMIMIMMTMMMMMMVMMMIMIMMMMMMATAMMMMMMMMMMTMTMMMIMWMRM |
| | | | MWMIMMMMMMMMMMMMMMMMMMMMMMMMMMMMMTMMMMMMMMMMMMMMMMMMMMMMMMMMMMMMMMMMMIVMMMMMMMMMMMMMM |
| | | | MMMMMMMMMMIMMMMMMIIMMMMMMMMMMMMMMMMMMMMMMMMMMMMIMMMMMMMMMMMIMMMMMMMMMMMMMM |
| 174 | D | 0.761 | # DDEDDDEDDD-GSDDDEDDDEEEDDDEEDEEDDDDEDEDDEEDDDEDEDDDDDEDEEEDEEXDEDEDEEDDDEDE |
| | | | DDEEEEEEEEDEEEDEE-DDDEEDEDDDDDEDDEDEDEEEDDDEEDDDEEDEEDDEDDDEDDDDDDDDDDDEDDDEDEDDEDEED |
| | | | DDEDDDEEDDEDDEDDDEEEDDDDEEEEEDDEDDDEEDDDDDEGDDDEDDEDDDDDDDDEDDDDDDDDDDDDDD |
| 175 | D | 0.962 | # DDDDDDDDDD-GDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDGDDDDDDDDDDDDDDDDDDDDDDD |
| 176 | P | 0.808 | # PPPPPPTPLPPQPPPPTPPPPPPPPPPPPPTPPPPPPTPPPTPPPKPPPPPPPPPPPPPTPPPTPTPPPPPPPPP |
| | | | PPPAPPPPPPPPPPPPPTPPPPPPPPPPLPPPPPPPPPPPPPPTKPPPTPPPPPPPPPPSPPPPPPPPPPPKPPPTPAPPPPPEP |
| | | | PPPPPPASPPPPPPPPPPPPPPPXPPPPXPPPPPPSPPPPPPPPPPPPPPPPPPPPPPPPPPPPPPPPPPPPPP |
| 177 | E | 0.953 | # EEEEEEEEEEEREEEEEEEEEEEEEEEEEEEEEEEEEEEEEEGEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEDEEEEEEEEEEEEEEEEEEEEEEEEEEEEEEEEEEEEEEEEEEE |
| 178 | R | 0.648 | # RKGKKKRKRRKKKRKGRKKKRKKRRRGRKRRRGKKRRRRGRGKRKKRRGKRRRRRRKKRGNKRRRRKGKRRKKKK |
| | | | KKRRRRKKKKRKKKKKKGKKKKRKRGRRKKRGRKKRKKKKRRKRGRKKKKKKRKRKKGRKKRKKKKKRGRKKKRKGKGRKRGGKR |
| | | | RRKRRRGRKKKKKGKKKRRKKRKRKRKKKKNKKKKGKKKKKRKKRGKKGRRKKGRGRRRRGGKGRRRGGRGGGG |
| 179 | E | 0.977 | # EEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEGEEEEEEEEEEEEEEEEEEEEEEEEDEEEEEEEEEEEEEEEEEEEEEEEEEEEKEEEEEEEEEEEEEEE |
| 180 | V | 0.961 | # VVVVVVVVVVVVVVVVVVVVVVVVVVVVVVVVVVVVVVVVVVVVVVGVVVVVVVVVVVVVVXVVVVVVVVVVVVV |
| | | | VVVVVVVVVVVVVVVVVVVVVVVVVVVVVVVVVVVVVVVVVVVVVVVVVVVVVVVVVVVVVVAVVVVVVVVVVVVVVVVVVVVVV |
| | | | VVVVVVVVVVVVVVVVVVVVVVVVVVVVVVVVVVVVVVVVVVVVVVVVVVVVVVVVVVVVVVVVVVVVVVVVVV |
| 181 | L | 0.994 | # LLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLLISILLLLLLLLLLLLLLLLLLLLLLLLLLLLLLLLLLLLLLLLLLLLLLLLLLLLLLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLLLL |
| 182 | E | 0.492 | # VVMMMQMVVEEMEVAQQMVMEVVVEEKQMEKEVMVVEKEVMMMVEMEEEVMMMVQEEEVREMQKVKEEVQVVVQE |
| | | | EMMVVVEVVVEVIVVMMRQMMEIKNEMMVVVQEQQMVVEAKEQVEEEEMVEMQVEQVEELMEEVMQVVMVMVEEVMMMMEMVMMQ |
| | | | EMMQVEEQMMVQXNVVVVMAAVVVAAMMEEEVVVVVVQQVMEVMVEVEEVVVVVVEVMVMEVVVMNVVVVEEVV |
| 183 | W | 0.972 | # WWWWW$WWWWWWWWWWWWWWWWWWWWWWWWWWRWWWWWWWWWWWWWWWWWWWWWWWWWWWWWWWWWWWWWWWWWW |
| | | | WWWWWWWWWWWWWWWWWGWWWWWWWWWWWWWWWWWWWWWWWWWWWWWWWWWWWWWWWWWWWWWWWWWWWWWWWWWWWWWWWWWWW |
| | | | WWWWWWWWWWWWWWWWWWWWWWWWWWWWWWWWWWWWWWWWWWWWWWWWWWWWWWWWWWWWWWWWWWWWWWWWWM |
| 184 | K | 0.742 | # RKKKKKKRKRRKRKRKKK$KRKKKRKKKKRKRKKKRRKRKKKKKRKRRKKKKKKKRRRKKRKKRRKRKKKRKRKR |
| | | | KKKKKKKRKKRKKRRKKSKKKKKKKRKKRKKKRKKKQRRKKRKKRKKKKKKKKKKRKRRKKRRKKKKKKKKKRRKKKKKRKKKKR |
| | | | RKKKKRKRKKKKKKERKKQQKRRKRKKKRKRRKRKKKKKKKRKKKRKRRRKKRRKRRKKKRKRKKKKRKRRRKR |
| 185 | F | 0.984 | # FFFFFFFLFFFFFFFFFFFFFFFFFFFFFFFFFFFFFFFFFFFFFFFPFFFFFFFFFFFFFFFFFFPFFFFFFFF |
| | | | FFFFFFFFFFFFFFFFFFFFFFFLFFFFFFFFFFFFFFFFFFFFFFFFFFFFFFFFFFFFFFFFFFFFFFFFFFFFFFFFFFFFF |
| | | | FFFFFFFFFFFFFFFFFFFFFFFFFFFFFFFFFFFFFFFFFFFFFFFFFFFFFFFFFFFFFFFFFFFFFFFFFF |
| 186 | D | 0.978 | # DDDDDDDDDDGDDDDDDDDDDDDDDDDDDDDDDDDDDDDDDDDDDDDDDDDDDDDDDDDDDDDDDDDDDDDDDDD |
| | | | DDDDDDDDDDDDDDDDDDDDDDDDDDDDDDDDDDDDDDDDDDDDDDDDDDDDDDDDDDDDDDDDDDDDDDDDDDDDDDDDDDDDD |
| | | | DDDDDDDDDDNDDDDDDDDDDDDDDDDDDDDDDDDDDDDDDDDDDDDEDDDDDDDDDDDDDDDDDDDDDDDDDD |
| 187 | S | 0.967 | # SSSSSSSSSSSSSSSSSSSSSSSSSSSSSRSSSSSSSSSSSSSSSSSSSSSSSSSSSSSSSSSSSSSSSSSSSSS |
| | | | SSSSSSSSSSSSSSSSSSSSSSSSSSSSSSSSSSSSSSSSSSSSSSSSSSSSSSSSSSSSSSSSSSSSSSSSSSSSSSSSSSSSS |
| | | | SSSSCSSSSSSSSSSSSSSSSSSSSSSSSSSSSSSSSSSSSSSSSSSSSSSSSSSSSSSSSSSSSSSSSSSSSS |
| 188 | R | 0.72 | # RRRRRRKHHRRSRHSRSRRRSRRRRRHRRRRRHRRRRRRHRHRRRRRRSRRRRHRRRRRXRRSHRMKRHRRHRRR |
| | | | RRHRRRRRSSRHRRRRRTTXRRRRRRRRRRRRRRRHRHRLRRHRRKRRRRKRRSRHHRRHRRHHRRRHRRSRRRHRRRRRRRRSR |
| | | | RRRRHRRRRRRRSRRRRRRRLRRHXRRHRGRRKHRRRRRRSRRRRRHRHRSRRRRRRRRRHRRRRRRRHRRRRR |
| 189 | L | 0.978 | # LLLLLLLLLLLLLPLLLLLLLLLLLLLLLLLLLLLLLLLLLLLLLLLLLLLLLLLLLLLLLXLLLLLLLLLLLLL |
| | | | LLLLLLLLLLLLLLLLLLLLLLLLLLLLLLLLLLLLLLLLLLLLLLLLLLLLLLLLLLLLLLLLLLLLLLLLLLLLLLLLLLLLL |
| | | | LLLLLLLLLLLLLLLXLLLLLLLLLLLLLLLLLLLLLLLLLLLLLLLLLLLLLLLLLLLLLLLLLLLLLLLLLL |
| 190 | A | 0.978 | # AAAAAAAAAAEAAAAAAAAAAAAAAAAAAAAAAAAAAAAAAAAAAAAAAAAAAAAAAAAAAAAAAAAAAAAAAAA |
| | | | AAAAAAAVAAAAAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAATAAAAAAAAAAAAAAAA |
| 191 | F | 0.867 | # FFFFFFFFFLFFFFFFFFFFFFFFLFFFFFFFFFFFFFFFFFLFFVFFFFIFFFFFFFYFFXFFFFFFHFFFFYF |
| | | | FFFFFFFLFFFFFFFFFFFFFFFFFFFFFFFFFFFFFFFFFFFFFFFFFFFFFFLFFFFFFFFFFFFFFFFFFFFFFFFFFFFFF |
| | | | FFFFFFFFFFFFFLFFFFFFFFFFFFFFFFFFCFFLFFFFFYFLFFYFFFFLFFFFFFFFFYFFFFFFFFFFYF |
| 192 | H | 0.777 | # HHHHHHHRRHHHHCHHHHHHHHHHHHRRHHPHRHHRHHHRHQRHHHNHHHHHHHRRRRHHHXHHKHHHRHHRNHR |
| | | | HHRQHHHRHHHRHRHHHHHHHHHHHQHHHRHHHHHRHRHHHRRHHHHHHHHHHHHKKHHHHHRRHQHHHHHHRHRHHHHHHHHHH |
| | | | HRHHRHHHHHHHHHHHHHHHHHRKHHHHHHHHHRHRHHHHHHHRHHHHRHHHHHHHHQHHRHHHHHHHHHHHHH |
| 193 | H | 0.966 | # HHHHHHHHHHNHNNHNHHHHHHHNHHHHHHHHHHHHHHHHHHHHHNNHHHHHHHHHHHHHHHHHHHHHHHHHNHH |
| | | | HHHHHHHHHHHHHHHHHHHHHHHHHHHHHHHHHHHHHHHHHHHHHHHHHHHHHHHHHHHHHHHHHHHHHHHHHHHHHHHHHHHHH |
| | | | RHHHHHHHHHHHHHHHHHHHHHHHHHHHHHHSHYHHHHHHHHHHHHHHHHHHHHHHHHHHHHHHHHHHHHHHHH |
| 194 | M | 0.63 | # VVMVMMVVVMMRKIMMVMMMKMMVMMMMMMMMMIRMMTIMMKFVIMVMRMMMMMMVMMMVMMVMMKVMMVMMMMM |
| | | | VMVMMMVMKKVMKMIMVVRMVMMMMMVMMMMMMVMMMMVVMMMMMVMMVMMMMRMMMMVKMMMMVMMMMVRMMMMMVVVVVVMVV |
| | | | MMVMVVMVMMMVVMMMMVMVVVVVVVVIVVVCVMMVMVMMRMMTMMMVMMMVVIMMKMMVMMMMIIMMMKMMMV |
| 195 | A | 0.988 | # APAAAAAAAAAAAAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAVAAAAAAAAAAAAAAAAAAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAAAAAAAAAAAAAAAAAAAAAAAAAAAAAA |
| 196 | R | 0.937 | # RDRRRRRRRRRRLRRRRRRRRRRRRRRRRRRRRRRRRRRRRRRRRRRRRRRRRRRRRRRRRRRRRRRRRRRRRRR |
| | | | RRRRRRRRRRRRRRQRRRRRRRRRRRRRRRRRRRRRRRRRRRRRRRRRRRRRRRRRRRRRKRRRRRKRRRRRRRRRRRRRRRRRR |
| | | | RRRRRRRRRRRRRRRRRRRRRRRRRRRRRRRCRRRRRRRRRRRRRRRRRRRRRRRRRRRRRRRRRRRRRRRRRR |
| 197 | E | 0.952 | # EEEEEEEEEEEEEEEEEEEEEEEEEEEEEEEEEEEEEEEEEEEEEEEEEEEEEEEEEEEEEEEEEEEEEEE$EEE |
| | | | EEDEEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEVVEEEEEEEEEEEEEEDEEEEEEEEEEEDEEEEEEEEEEEEEE |
| 198 | L | 0.658 | # LLKLLLKLLLLMLLLLQLKKLKLLLLLMKLLRLKLLLLKLLMKTILLLLLMKKKLILLLLLXLLLLLLILLLLLL |
| | | | LKLLLLLLLLLLLLLKKLNLMLLLLLKKLVLQMMLLLLMLLLKLKLLLKMLKLLLLVLKLKLLLLLLLLLLKLLLLILLQLLLKM |
| | | | KLQLLLKLKKLLLLKKLLKQLXTLMLLLLLLFLLKLLLKLLLLLLKKLLKKKLKKKLKKLLKKKKKKKKLKKKK |
| 199 | H | 0.948 | # HHHHHHHYHHHHHHHHHHHHHHHHHHHHYHHHHHHHHHHHHHHHHHHHHHHHHHHHHHHHHHHHHHHHHHHHHHH |
| | | | HHHHHHHHHHHHHHHHHHHHHHYHHHHHHHHHHHHHHHHHHHHHHHHHHHHHHHHHHHHHHHHHHHHHHHYHHHHHHHRHHHHHH |
| | | | HHHHHHHHHHHHHHHHHHHHHHHQHHHHHHHHHHHHHHHHHHHHHHHHHHHHHHYHHHYHHHHHHHHHHHHHHH |
| 200 | P | 1 | # PPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPPPPPPPPPPPPP |
| | | | PPPPPPPPPPPPPPPPPPPPPPPPPPPPPPPPPPPPPPPPPPPPPPPPPPPPPPPPPPPPPPPPPPPPPPPPPP |
| 201 | E | 0.956 | # EEEDDEEEEEDEEEEEEEEEEEEEEEEEEEEEEEEEEEEEEEEEEEEEEXEEEEEEEEEEEEEEEEEEEEEEEEE |
| | | | EEEEEEEEEEEEEEEEEEEEEEEEEEEEEEEEEEEEEEEEEEEEEEEEEEEEEEEEEEEEEEEEEEEEEEEEEEEEEEEEEEEEE |
| | | | EEEEEEEEEEEEEEEEEEEEEEEEEEEEEEEEEEEEEDEEEEEEEEEEEEEEEEEEEEEEEEEEEEEEEEEEEE |
| 202 | Y | 0.842 | # YYYYYYFYYYYYYYYYYYYYYYYYYYYYYYYYYYYYYYYYYFFYYYYYYSYYYYYYYYYYYFYYYYYYYYYYYYY |
| | | | YYYYYYYYYYYYYYYYFFYYYYYYYYYFYYYYYYYYYYYYYYYYYYYYFYYYHYYYYFYYFYYYYYFYYYYYYYYYYYYYYYYYY |
| | | | YYYYYYYYYFFFYYFFYYYYYYFYYYYYYYYYYYFYYYYYYYYYYYYYYYYSYYYYYYYYYYYYYYFYYYFYYF |
| 203 | Y | 0.859 | # YYYYYYYYYYFYFYYFYYYYFFYYYYYYYSYFYYYYYYYYYYYYYYYYYSHYYYYYYYYYYYYYYYYYYYYYYYY |
| | | | YYYYYYYYYYFYYYYYYCYYYYYYYYYYYYYFYYYYYYYYYYYYYYYYYYFYYYYYYYYYYYYYYFYYVYYYYYYYYYYYYYYYY |
| | | | FYYYFFFYYYYYYYYYYYYYYYYYFYYYYYYYYYYYFYFFYFYYFFYYYYYYYYYYYYYYYYYYYYYYYYYFYY |
| 204 | K | 0.948 | # KKKKKKKKKKKTKKKIKKKKKKKKKKKKKKKKKKKKKKKKKKKKKKKKKTKKKKKKKKKKKKKKKKKKKKKKKKK |
| | | | KKKKKKKKKKKKKKKKKKKKKKKKKKKKKKKKKKKKKKKKKKKKKKEKKKKKKKKKKKKKKKKKKKKKKKKKKKKKKKKKKKKKK |
| | | | KKKSKKKKKKKKKKKKKKKKKKKKQKKKKKKKKKKKKKKKKKKKKKKKKKKKKKKKKKKKKKQKKKKKKKKKKK |
| 205 | D | 0.741 | # NDDNDNDNNNNNDNNDNDDDNDN-NNDDNNDNNNDNNDNNNDDDDNNDNDDDDDDDDDDXDDDDNDDDNNDNDND |
| | | | NNNDNNNDDDNDNDSDDDNNDNDDDDDDDDNDNNNDNDNDDDDDNNNNNNNDDNDNNDNNDNNDNDNDNDNDDDDDNDNNNNNDN |
| | | | NDNNNNNNDDDDNNNXDNNNDNDDNDDDDDDDDNDDDEDDNNDNNDDNDNDDNDNDNDNNDNNDNNNDNNDDND |
| 206 | C | 0.855 | # $CCCCSC$$CC$R$CCCCCCCCCICCCCCCC$CCC$CCCCCCCCC$$CCCCCCCCCCCC$CCCC$CCCCCCCCCC |
| | | | CCCCCCCCCCCCCCCCCCCCCCCCCCCCCCCCCCCCCC$CCCCCCCCCCCCCC$CCCCCCCCCCCCNSCC$CCCCC$CC$$$CCC |
| | | | CCCRCCCCCCCCCCCCCCCCCCCCCCCCCCCCCCCCCLCCCCCCCCCCKCCCCCCCCCCCCCCCS$CCCCCCCC |
| | C | 0.994 | # -$$$$$$--$$-$$$$$$$$$$$$$$$$$$$$$$$$$$$$$$$$$--$$$$$$$$$$$$-$$$$-$--------- |
| | | | -------$$$$------------------$$$$$$$$$-$$$$$$$$$$$$$$-$$$$$$$$$$$$$-$$-$$$$$-$$---$$$ |
| | | | $$$$$$$$$$$$$$$$$$$$$$$$$$$$$$$$$$$$$X$$$$$$$$$$D$$$$$$$$$$$$$$$$$$$$$$$$$ |
| | | 0.855 | |
| | | 0.994 | |

TABLE 10
Experimental proof of differences between stable
vs escaping average epitope conservation values

| | |
|---|---|
| Number of epitopes examined: | 57 |
| Average Shannon conservation value | 94.50% |
| of stable epitopes | |
| Average Shannon conservation value | 90.50% |
| of escaping epitopes | |
| Statistical p value of conservation | p = <0.0005 |
| difference | |

TABLE 11

| | | | | | | | |
|---|---|---|---|---|---|---|---|
| Experimental | | | | | | | |
| proof of | | | | | | | |
| differences | | | | | | | Non-fixed |
| between stable | | | | | | | amino acid |
| vs escaping | | | | | | | (Observed |
| individual | | | | | | Shannon | amino acid |
| residue | | Unfixed epitopes | (Published epitope | HIV clade B | Shannon | conservation | change |
| conservation | Source of epitope | region (Amino | region amino acid | consenses | conservation value | value (non-fixed | observed in |
| values | sequence data | acid number) | sequence) | sequence | (fixed residues) | residues) | patients) |

| | | | | | |
|---|---|---|---|---|---|
| Observed | Pol (838) | S | S | 0.937 | |
| epitope escape in | | T | T | | 0.744 T to N |
| Gag and Pol | | T | T | | 0.83 T to A |
| proteins. | | V | V | 1 | |
| | | K | K | 0.937 | |
| | | A | A | 1 | |
| | | A | A | 1 | |
| | | C | C | 1 | |
| | | W | W | 0.963 | |
| | | W | W | 0.937 | |
| | Pol IW9 (399) | I | I | 1 | |
| | | V | V | | 0.784 V to M |
| | | L | L | 1 | |
| | | P | P | 1 | |
| | | E | E | 0.963 | |
| | | K | K | 1 | |
| | | D | D | 0.963 | |
| | | S | S | 1 | |
| | | W | W | 0.963 | |
| | AK11 Gag (349) | A | A | 1 | |
| | | C | C | 1 | |
| | | Q | Q | 1 | |
| | | G | G | 0.973 | |
| | | V | V | 0.973 | |
| | | G | G | 0.973 | |
| | | G | G | 1 | |
| | | P | P | 1 | |
| | | G | G | | 0.726 G to S |
| | | H | H | 1 | |
| | | K | K | 1 | |
| | GL9 Gag (71) | G | G | 1 | |
| | | S | S | 0.931 | |
| | | E | E | 1 | |
| | | E | E | 0.949 | |
| | | L | L | 0.777 | |
| | | R | R | 0.763 | |
| | | S | S | 1 | |
| | | L | L | 0.886 | |
| | | Y | Y | | 0.736 Y to F |
| | AK9 Pol (313) | A | A | 1 | |
| | | I | I | 1 | |
| | | F | F | 1 | |
| | | Q | Q | 1 | |
| | | S | S | 0.805 | |
| | | S | S | 1 | |
| | | M | M | 1 | |
| | | T | T | 1 | |
| | | K | K | | 0.861 K to I |
| Hay C et. al., | Pol (311) | S | S | 1 | |
| Journal of | | P | P | 1 | |
| Virology, Vol.73, | | A | A | 1 | |
| p 5509, 1999. | | I | I | 1 | |
| | | F | F | 1 | |
| | | Q | Q | 1 | |
| | | S | S | | 0.805 S to C |
| | | S | S | 1 | |
| | | M | M | 1 | |
| | | | Sample | 50 | |
| | | | number (fixed) | | |
| | | | Sample | | 7 |
| | | | number (non- | | |
| | | | fixed) | | |
| | | | Mean of X | 0.97386 | |
| | | | Mean of Y | 0.783714 | |
| | | | Standard | 0.05527 | |
| | | | Deviation of X | | |
| | | | Standard | 0.05124 | |
| | | | Deviation of Y | | |
| | | | Large Sample | 0.020885 | |
| | | | Standard | | |
| | | | Deviation | | |
| | | | t-statistic | 9.10447 | |
| | | | degrees-of- | 8.087577 | |
| | | | freedom | | |
| | | | p-value | 0.000008 | |
| | | | Statistical | | |
| | | | methodology: T | | |
| | | | test of | | |
| | | | differences of | | |
| | | | means with | | |
| | | | variance | | |
| | | | assumed | | |
| | | | equal. | | |

Note:
fixed residue avg. conservation values are statistically different than non-fixed avg. conservation values at a significance level of 5%.

TABLE 12

| | | | | | | | |
|---|---|---|---|---|---|---|---|
| | | | | | | | Amino acid variablility; (Amino acids at |
| | Unfixed | | | | Shannon | Non fixed amino | given position in database/number of HIV |
| Source of | epitopes | (Published | | Shannon | conservation | acid (Observed | strains examined in database). Note that |
| epitope | region | epitope region | HIV clade B | conservation | value (non- | amino acid | experimentally observed amino acids |
| sequence | (Amino acid | amino acid | consenses | value (fixed | fixed | change observed | mutate toward amino acid observed in |
| data | number) | sequence) | sequence | residues) | residues) | in patients) | database in 5/6 cases. |

| | | | | | | |
|---|---|---|---|---|---|---|
| Observed | Pol (838) | S | S | 0.937 | | |
| epitope | | T | T | | 0.744 T to N | T (29/41), A (8/41), N (4/41) |
| escape in | | T | T | | 0.83 T to A | T (34/41), A (6/41), S (1/41) |
| Gag and | | V | V | 1 | | |
| Pol | | K | K | 0.937 | | |
| proteins. | | A | A | 1 | | |
| | | A | A | 1 | | |
| | | C | C | 1 | | |
| | | W | W | 0.963 | | |
| | | W | W | 0.937 | | |
| | Pol IW9 | I | I | 1 | | |
| | (399) | V | V | | 0.784 V to M | V (33/41), M (5/41), T (2/41), K (1/41) |
| | | L | L | 1 | | |
| | | P | P | 1 | | |
| | | E | E | 0.963 | | |
| | | K | K | 1 | | |
| | | D | D | 0.963 | | |
| | | S | S | 1 | | |
| | | W | W | 0.963 | | |
| | AK11 Gag | A | A | 1 | | |
| | (349) | C | C | 1 | | |
| | | Q | Q | 1 | | |
| | | G | G | 0.973 | | |
| | | V | V | 0.973 | | |
| | | G | G | 0.973 | | |
| | | G | G | 1 | | |
| | | P | P | 1 | | |
| | | G | G | | 0.726 G to S | G (42/63), S (17/63), H (1/63) |
| | | H | H | 1 | | |
| | | K | K | 1 | | |
| | GL9 Gag | G | G | 1 | | |
| | (71) | S | S | 0.931 | | |
| | | E | E | 1 | | |
| | | E | E | 0.949 | | |
| | | L | L | 0.777 | | |
| | | R | R | 0.763 | | |
| | | S | S | 1 | | |
| | | L | L | 0.886 | | |
| | | Y | Y | | 0.736 Y to F | Y (40/63), F (20/63), H (3/63) |
| | AK9 Pol | A | A | 1 | | |
| | (313) | I | I | 1 | | |
| | | F | F | 1 | | |
| | | Q | Q | 1 | | |
| | | S | S | 0.805 | | |
| | | S | S | 1 | | |
| | | M | M | 1 | | |
| | | T | T | 1 | | |
| | | K | K | | 0.861 K to I | K (36/41), R (4/41), T (1/41) |
| Hay C et. | Pol (311) | S | S | 1 | | |
| al., Journal | | P | P | 1 | | |
| of Virology, | | A | A | 1 | | |
| Vol.73, p | | I | I | 1 | | |
| 5509, 1999. | | F | F | 1 | | |
| | | Q | Q | 1 | | |
| | | S | S | | 0.805 S to C | S (32/41), C (8/41), Y (1/41) |
| | | S | S | 1 | | |
| | | M | M | 1 | | |

What is claimed is:

1. An immunogenic composition comprising two or more peptides, wherein said peptides are substantially identical epitopes of an HIV-1 protein, said epitopes comprising an amino acid sequence with at least one amino acid position having an entropy score less than the average entropy score of said HIV-1 protein, and wherein said peptides have a different amino acid at said amino acid position with low entropy score.

2. The composition of claim 1, wherein said entropy score is determined by ΣPₐₐlogPₐₐ, where Pₐₐ is the proportion of each amino acid in the respective position.

3. The composition of claim 1, wherein said entropy score is 0.900 or less.

4. The composition of claim 3, wherein said entropy score is 0.800 or less.

5. The composition of claim 4, wherein said entropy score is 0.700 or less.

6. The composition of claim 1, wherein said HIV-1 protein is Pol, Vif, Tat, Rev, Vpu, Env, Vpr, or Nef.

7. The composition of claim 6, wherein said HIV-1 protein is a Pol protein.

8. The composition of claim 7, comprising a peptide having the amino acid sequence STTVKAACWW (SEQ ID NO: 1) and at least one peptide having an amino acid sequence selected from the group consisting of SNTVKAACWW (SEQ ID NO: 7), SATVKAACWW (SEQ ID NO: 8), STAVKAACWW (SEQ ID NO: 9), STSVKAACWW (SEQ ID NO: 10), or combinations thereof.

9. The composition of claim 7, comprising a peptide having the amino acid sequence IVLPEKDSW (SEQ ID NO: 2) and at least one peptide having an amino acid sequence selected from the group consisting of IMLPEKDSW (SEQ ID NO: 11), ITLPEKDSW (SEQ ID NO: 12), IKLPEKDSW (SEQ ID NO: 13), or combinations thereof.

10. The composition of claim 7, comprising a peptide having the amino acid sequence AIFQSSMTK (SEQ ID NO: 3) and at least one peptide having an amino acid sequence selected from the group consisting of AIFQSSMTR (SEQ ID NO: 14), AIFQSSMTT (SEQ ID NO: 15), AIFQSSMTI (SEQ ID NO: 16), or combinations thereof. and.

11. The composition of claim 7, comprising a peptide having the amino acid sequence SPAIFQSSM (SEQ ID NO: 4) and at least one peptide having an amino acid sequence selected from the group consisting of SPAIFQCSM (SEQ ID NO: 17) or SPAIFQYSM (SEQ ID NO: 18).

12. The composition of claim 6, wherein said HIV-1 protein is a Gag protein.

13. The composition of claim 12, comprising a peptide having the amino acid sequence ACQGVGGPGHK (SEQ ID NO: 5) and a peptide having the amino acid sequence ACQGVGGPSHK (SEQ ID NO: 19).

14. The composition of claim 7, comprising a peptide having the amino acid sequence GSEELRSLY (SEQ ID NO: 6) and a peptide having the amino acid sequence GSEELRSLF (SEQ ID NO: 20).

15. A method of identifying an escape mutation site in an HIV-1 protein, comprising the steps of:
(a) determining a first entropy score for a first amino acid position of said protein; and
(b) comparing said first entropy score to a second entropy score, said second entropy score is the entropy score for a second amino acid position of said protein or an average entropy score for 2 or more amino acid positions in said protein, wherein a lower entropy score at said first amino acid position relative to said second entropy score identifies an escape mutation at said first amino acid position.

16. A method of determining the composition of an HIV-1 vaccine, said vaccine is an escape mutation resistant vaccine, comprising the steps of:
(a) providing an entropy score profile of an HIV-1 protein;
(b) selecting an amino acid sequence within said HIV-1 protein comprising an amino acid position with a low entropy score;
(c) determining the identity of amino acid variants at said amino acid site from a patient-derived index of amino acid sequences of said HIV-1 protein; and
(d) selecting at least peptide comprising an amino acid sequence that comprises an amino acid variant at said amino acid position with low entropy score.

17. The method of claim 16, wherein said low entropy score is 0.900 or less.

18. A method of determining the composition of an HIV-1 vaccine, said vaccine is an escape mutation resistant vaccine, comprising the steps of:
(a) providing an entropy score profile of an HIV-1 protein;
(b) selecting an amino acid sequence within said HIV-1 protein comprising an amino acid position with a low entropy score;
(c) determining the identity of amino acid variants at said amino acid site from a patient-derived index of amino acid sequences of said HIV-1 protein; and
(d) selecting at least two substantially identical peptides, wherein said peptides comprise said amino acid sequence with said amino acid position with low entropy score and wherein said peptides have different amino acids at said amino acid position.

19. The method of claim 18, wherein said low entropy score is 0.900 or less.


SEQUENCE LISTING (230 sequences)

SEQ ID NO: 1 - protein, 10 aa, Homo sapiens
STTVKAACWW

SEQ ID NO: 2 - protein, 9 aa, Homo sapiens
IVLPEKDSW

SEQ ID NO: 3 - protein, 9 aa, Homo sapiens
AIFQSSMTK

SEQ ID NO: 4 - protein, 9 aa, Homo sapiens
SPAIFQSSM

SEQ ID NO: 5 - protein, 11 aa, Homo sapiens
ACQGVGGPGHK

SEQ ID NO: 6 - protein, 9 aa, Homo sapiens
GSEELRSLY

SEQ ID NO: 7 - protein, 10 aa, Homo sapiens
SNTVKAACWW

SEQ ID NO: 8 - protein, 10 aa, Homo sapiens
SATVKAACWW

SEQ ID NO: 9 - protein, 10 aa, Homo sapiens
STAVKAACWW

SEQ ID NO: 10 - protein, 10 aa, Homo sapiens
STSVKAACWW

SEQ ID NO: 11 - protein, 9 aa, Homo sapiens
IMLPEKDSW

SEQ ID NO: 12 - protein, 9 aa, Homo sapiens
ITLPEKDSW

SEQ ID NO: 13 - protein, 9 aa, Homo sapiens
IKLPEKDSW

SEQ ID NO: 14 - protein, 9 aa, Homo sapiens
AIFQSSMTR

SEQ ID NO: 15 - protein, 9 aa, Homo sapiens
AIFQSSMTT

SEQ ID NO: 16 - protein, 9 aa, Homo sapiens
AIFQSSMTI

SEQ ID NO: 17 - protein, 9 aa, Homo sapiens
SPAIFQCSM

SEQ ID NO: 18 - protein, 9 aa, Homo sapiens
SPAIFQYSM

SEQ ID NO: 19 - protein, 11 aa, Homo sapiens
ACQGVGGPSHK

SEQ ID NO: 20 - protein, 9 aa, Homo sapiens
GSEELRSLF

SEQ ID NO: 21 - protein, 502 aa, Homo sapiens
MGARASVLSGGELDRWEKIRLRPGGKKKYRLKHIVWASRELERFALNPGLLETSAGCRQILGQLQPSLQTGSEELRSLYNTVATLYCVHQKIEVRDTKEALDKIEEEQNKSKKKAQQAAADTGNSSQVSQNYPIVQNLQGQMVHQAISPRTLNAWVKVVEEKAFSPEVIPMFAALSEGATPQDLNTMLNTVGGHQAAMQMLKETINEEAAEWDRMHPVHAGPIAPGQMREPRGSDIAGTTSNLQEQIGWMTNNPPIPVGEIYKKWIIMGLNKIVRMYPTSILDIRQGPKEPFRDYVDRFYKTLRAEQATQEVKNWMTETLLVQNANPDCKTILKALGPAATLEEMMTACQGVGGPGHKARVLAEAMSQVTSPATIMMQRGNYKNQRKTVKCFNCGREGHIAKNCRAPRKKGCWKCGKEGHQMKDCTERQANFLGKIWPSHKGRPGNFLQSRPEPTAPPAPPEESFRFGGETATPSQKQEPIDKEMYPLASLRSLFGNDPSSQ

SEQ ID NO: 22 - protein, 1002 aa, Homo sapiens
FFRENLAFPQGEAREFPPEQTRANSPTSPTRGELQVWGRDSNSLSEAGADRQGNVSFGFPQITLWQRPIVTIKIGGQLKEALLDTGADDTVLEEMNLPGRWKPKMIGGIGGFIKVRQYDQIPIEICGRAVGTVLVGPTPVNIIGRNLLTQIGCTLNLPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDMVIYQYMDDLYVGSDLEIGQHRTKIEELREHLLKWGFTTPDKKHQKEPPFLWMGYELHPDKWTVQPIVLPEKDSWTVNDIQKLVGKLNWASQIYGIKVKQLCKLLRGAKALTEVVPLTEEAELELAENREILKEPVHGVYYDPSKDLIAEIQKQGQCQWTYQIYQEPFKNLKTGKYARTRGAHTNDVKQLTEAVQKIATESIVIWGKTPKFRLPIQKETWESWWTEYWQATWIPEWEFVNTPPLVKLWYQLEKEPIVGAETFYVDGASNRETKVGKAGYVTDRGRQKVISLTDTTNQKTELQAIHLALQDSGSEVNIVTDSQYALGIIQAQPDKSESEIVSQIIEQLINKEKVYLAWVPAHKGIGGNEQVDKLVSAGIRKILFLDGIDKAQEEEKYHSNWKAMASDFNLPPVVAKEIVASCDKCQLKGEAMHGQVDCSPGIWQLDCTHLEGKIILVAVHVASGYIEAEVIPAETGQETAYFILKLAGRWPVKTIHTDNGPNFISTTVKAACWWAGIKQEFGIPYNPQSQGVVESMNKELKKIGQVRDQAEHLKTAVQMAVFIHNFKRKGGIGEYSAGERIIDIIATDIQTKELQKQITKIQNFRVYYRDSRDPLWKGPAKLLWKGEGAVVIQDNSDIKVVPRRKAKIIRDYGKQMAGDDCVASRQDED

SEQ ID NO: 23 - protein, 191 aa, Homo sapiens
MENRWQVMIVWQVDRMRIRTWKSLVKHHMYVSKKAKGWIYKHHYESTHPRISSEVHIPLGDARLVVTTYWGLHTGERDWHLGQGVSIEWRKKRYSTQVDPDLADQLIHLYYFDCFSETAIRNAILGHVSPRCEYQAGHNKVGSLQYLALAALVTPKRIKPPLPSVTKLTEDRWNKPQKTKGHRGSHTMNGH

SEQ ID NO: 24 - protein, 96 aa, Homo sapiens
MEQAPENQGPQREPYNEWTLEILEELKNEAVRHFPRIWLHSLGQHIYETYGDTWAGVEAIIRTLQQLLFIHFRIGCQHSRIGITRQRRARNGASRS

SEQ ID NO: 25 - protein, 102 aa, Homo sapiens
MEPVDPRLEPWKHPGSQPKTACTTCYCKKCCFHCQVCFITKGLGISYGRKKRRQRRRAPQDSQTHQASISKQPTSQPRGDPTGPKEPKKKVERETETHPVDQ

SEQ ID NO: 26 - protein, 117 aa, Homo sapiens
MAGRSGDSDEELLKTGRLIKLLYQSDPPPSPEGTRQARRNRRRRWRERQRHIREISGWILDTYLGRSAEPAPLQLPPLERLTLDCSEGCGTSGTQGVGGPQILVESPAILESGTKEE

SEQ ID NO: 27 - protein, 79 aa, Homo sapiens
MHSLTIVAIVALVVAILIAIVVWSIVFIEYRKILRQRKLDRLIERIRERAEDSGNESDGDQEELLVEMGHLAPWDINDL

SEQ ID NO: 28 - protein, 219 aa, Homo sapiens
MGGKWSKGIGRGWPAVRERMRRAAPADGVGTASQRAEPAADGVGAASKDLEKHGAITSSNLAANNADVAWLEAQEEEEVGFPVRPQVPLRPMTYKGALDLSHFLKEKGGLEGLVWSQKRQDILDLWVYHTQGYFPDWQDYTPGPGVRYPLTFGWCFKLVPVDPDEVEEANEGENNCLLHPISLHGMEDSEKEVLVWKFDSHLAFRHMARELHPEYYKNC

SEQ ID NO: 29 - protein, 503 aa, Homo sapiens
MGARASVLSGGELDRWEKIRLRPGGKKKYRLKHIVWASRELERFALNPSLLETSEGCRQILGQLQSSLQTGTEEIKSLYNTVATLYCVHQRIDVKDTKEALDKIEEEKNKSKKKAQAEQAAAGTGNSSQVSQNYPIVQNMQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIKQGPKEPFRDYVDRFYRTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIARNCRAPRKKGCWKCGKEGHQMKDCTNERQANFLGKIWPSSKGRPGNFLQSRPEPTAPPEESFRFGEETTTPPQKQETIDQEKYPLASLKSLFGSDPLSQ

SEQ ID NO: 30 - protein, 998 aa, Homo sapiens
FFREDLAFLQGEAREFSSEQARANSPTRRELQVWGRDNNSPSEAGDDRPGKVSLSFPQITLWQRPLVTIKIGGQLKEALLDTGADDTVLEDINLPGRWKPKMIGGIGGFIKVRQYDQVSIEICGHKAVGTVLVGPTPVNIIGRNLLTQIGCTLNFPISPIDTVPVKLKPGMDGPKVKQWPLTEEKIKALIEICAEMEKEGKISKIGPENPYNTPVFAIKKKDSTKWRKLVDFRELNKKTQDFWEVQLGIPHPAGLKKKKSVTVLDVGDAYFSVPLDDFRKYTAFTIPSINNETPGIRYQYNVLPQGWKGSPAIFQSSMTKILEPFRKQNPDIVIYQYMDDLYVGSDLEIGQHRIKIEELRDHLLKWGFTTPDKKHQKEPPFLWMGYELHPDKWTVQPIALPEKDSWTVNDIQKLVGKLNWASQIYGIKVRQLCKLLRGTKALTEVVPLTEEAELELAENREILREPVHGVYYDPSKDLIAEIQKQEHGQWTYQIYQEPFKNLKTGKYARMRGAHTNDVKQLTEAVQKIANECIVIWGKTPKFRLPIQKETWETWWTEYWQATWIPEWEFVNTPPVKLWYQLEKEPIEGAETFYVDGAANRETKLGKAGYVTNRGRQKVVPLTDTTNQKTELQAIYLALQDSGSEVNIVTDSQYSLGIIQAQPDKSESEIVSQIIEQLIKKEKVYLAWVPAHKGIGGNEQVDKLVSAGIRRVLFLDGIDKAQEDEKYHSNWRAMASDFNLPPIVAKEIVASCDKCQLKGEAMHGQVDCSPGIWQLDCTHLEGKIVLVAVHVASGYIEAEVIPAETGQETAYFILKLAGRWPVKTIHTDNGPNFISTTVKAACWWAGIKQEFGIPYNPQSQGVVESMNKELKKIGQVRDQAEHLKTAVQMAVFIHNFKRKGGIGGYTAGERIVDIIATDIQTKELQKQITKIQNFRVYFRDNRDPLWKGPAKLLWKGEGAVVIQDNNEIKVVPRRKAKIIRDYGKQMAGDDCVASGQDED

SEQ ID NO: 31 - protein, 191 aa, Homo sapiens
MENRWQVMIVWQVDRMRISTWKSLVKHHMYVSKKAKGWVYKHHYEGTHPRISSEVHIPLGDAKLIVTTYWGLHTGEREWHLGQGVSIEWRKRRYSTQVDPGLADQLIHMYYFDCFSDSAIRRAIIGHVSPSCEYQAGHNKVGSLQYLALAALVTPRKIKPPLPSIAKLTEDRWNKPHKTKGHRGSHTMTGH

SEQ ID NO: 32 - protein, 96 aa, Homo sapiens
MEQAPQDQGPPREPYHDWTLEILEELKNEAVRHFPRIWLHGLGQQIYGTYGDTWEGVEAIIRTLQQLLFIHFRIGCQHSRIGIIRQRRTRNGASRS

SEQ ID NO: 33 - protein, 101 aa, Homo sapiens
MEPVDPSLEPWKHPGSQPKTACTPCYCKKCCFHCQVCFMTKGLGISYGRKKRRQRRRAPQGHPNNQVSISKQPASQPRGDQTGPKESKKKVESKTETDQVS

SEQ ID NO: 34 - protein, 117 aa, Homo sapiens
MAGRSGDSDEELLKAIRLIKFLYQSDPPPSPEGTRQARRNRRRRWRARQRQIRSLSGWILSTHLGRPTEPVPFQLPPLERLTLDCSEDCGTSGTQGVGSSPVLVESHTVLDSGTKEE

SEQ ID NO: 35 - protein, 81 aa, Homo sapiens
VMSLYIISIVALVIAAIIAIVVWSIVFIEYRKILRQRKIDRLIDRIRDRAEDSGNESEGDQEELAALVEMGHDAPWVVDDL

SEQ ID NO: 36 - protein, 207 aa, Homo sapiens
MGGKWSKSSLIGWPRIRDRMRRTEPEPAAVGVGAVSRDLEKHGAITSSNTAATNAECAWLEAQEEEEVGFPVRPQVPLRPMTYKAAVDISHFLKEKGGLEGLIHSQKRQDILDLWVYHTQGYFPDWQYTPGPGVRYPLTFGWCFKLVPVEPEDVERANEGGNNCLLHPMSQHGMDDPEREVLQWKFDSRLAFHHMARELHPEYYKDC

SEQ ID NO: 37 - protein, 502 aa, Homo sapiens
MGARASVLSGGKLDRWEKIRLRPGGKKKYKLKHIVWASRELERFAVNPGLLETAEGCRQILGQLQPSLQTGSEELKSLFNTVATLYCVHQNIEVKDTKEALDKVEEEQNKSKKKAQQAAADTGNNSQSQNYPIVQNLQGQMVHQALSPRTLNAWVKVVEEKNFSPEVIPMFTALSEGATPQDLNTMLNTVGGHQAAMQMLKETINEEAAEWDRLHPVAAGPVAPGQIREPRGSDIAGTTSNLQEQIAWMTHNPPIPVGEIYKRWIILGLNKIVRMYPTSILDIKQGPKEPFRDYVDRFYKTLRAEQASQDVKNWMTETLLVQNANPDCRTILKALGPAATLEEMMAACQGVGGPGHKARVLAEAMSQVTNSATIMMQKGNFRGQRKIVKCFNCGKEGHIAKNCRAPRKRGCWKCGKEGHQMKDCTRQANFLGKIWPSSKGRPGNFLQSRPEPTAPTAPTAPPEESFRFGEEMTPSQKQERIDKEPSPLASLRSLFGSDPSSQ

SEQ ID NO: 38 - protein, 1002 aa, Homo sapiens
FFREDLAFLQGKAREFSSEQTRANSPNSPNSPTRGELQVWGRDDSLSEAGADRQGTVSFSFPQITLWQRPLVTIKIGGQLKEALLDTGADDTVLEEMNLPKRWKPKMIGGIGGFIKVRQYDQILIEIGHKAIGAVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDDFRKYTAFTIPSINNETPGIRYQYNVLPQGWKGSPAIFQSSMTKILEPFRKQNPEIVIYQYMDDLYVGSDLEIEQHRTKIEELRQHLLRWGFTTPDKKHQKEPPFLWMGYELHPDKWTVQPIKLPEKDSWTVNDIQKLVGKLNWASQIYGIKVKQLCKLLRGTKALTEVVPLTEEAELELAENREILKEPVHGVYYDPSKDLIAEIQKQEQGQWTYQIYQEPFKNLKTGKYARTRGAHTNDVKQLTEVVQKIAREAVVVWGKIPKFRLPIQKETWEAWWMEHWQATWIPEWEFVNTPPVKLWYQLEKEPIVGAETFYVDGAANRETKLGKAGYVTDRGRQKVVFLTDTTNQKTELQAIHLALQDSGLEVNIVTDSQYALGIIQAQPDKSESELVSQIIEQLIRKEKVYLAWVPAHKGIGGNEQVDKLVSTGIRKVLFLDGIDKAQEEEKYHNNWRAMASDFNLPPVVAKEIVASCDKCQLKGEAMHGQVDCSPGIWQLDCTHLEGKIILVAVHVASGYIEAEVIPAETGQETAYFLLKLAGRWPVKVIHTDNGGNFISNAVKAACWWAGIKQEFGIPYNPQSQGVVESMNKELKKIGQVRDQAEHLKTAVQMAVFVHNFKRKGGIGGYSAGERIIDIIATDIQTKELQKQITKIQNFRVYYRDSRDPLWKGPAKLLWKGEGAVVIQDNSDIKVVPRRKVKIIRDYGKQMAGDDCVASRQDED

SEQ ID NO: 39 - protein, 191 aa, Homo sapiens
MENRWQVMIVWQVDRMRIRTWNSLVKHHMYKSRKAKGWCYKHHYESTNPRISSEVHIPLKDAKLVITTYWGLHTGEQDWHLGQGVSIEWRERSYRTQVDPGLADQLIHLHYFDCFSESAIRKTIVGHVSPRCEYQAGHNKVGSLQYLALAALITPKKIKPPLPSVRKLTEDRWNKPQKTKGHRGSHTMNGH

SEQ ID NO: 40 - protein, 96 aa, Homo sapiens
MEQAPEDQGPQREPHNEWTLELLEELKSEAVRHFPRIWLHNLGQHIYETYGDTWAGVEALIRILQQLLFIHFRIGCQHSRIGILQRRRARNGASRS

SEQ ID NO: 41 - protein, 101 aa, Homo sapiens
MEPVDPRLEPWKHPGSQPKTACTNCYCKKCCFHCQVCFLKKGLGISYGRKKRRQRRRPPPGSQNNQVSLSKQPASQPRGDPTGPKESKKKVERETETDPVD

SEQ ID NO: 42 - protein, 117 aa, Homo sapiens
MAGRRGDSDEDLLQAVRIIKFLYQNDPPPNPEGTRQARRNRRRRWRERQRQIQSISAWIISTHLGRSAEPVPLQLPPLERLTLDCSEDCGTSGTQGVGSPQVLGESPTVLESGAKEE

SEQ ID NO: 43 - protein, 81 aa, Homo sapiens
MQSLETIAIVALVVAIIIAIVVWSIVLIEYRKILRQRKIDRLIDRIRERAEDSGNESEGDEEELAGLVERGHLAPGNIDDL

SEQ ID NO: 44 - protein, 207 aa, Homo sapiens
MGGKWSKSKRSEWSAVREKMQQAEPAAEPAAEGVGAVSRDLEKHGAVTSNNLNNAACTWLEAQEQEEEVGFPVRPQVPLRPMTYKGALDLSHFLKEKGGLEGIIYSQQRQDILDLWVYNTQGYFPDWNYTPGPGIRYPLTFGWCFKLVPVTEVDVEANQGENSKLLHPMSLNGMEDPEKEVLQWKFDSRLAFHHMAREKHPEYFKDC

SEQ ID NO: 45 - protein, 497 aa, Homo sapiens
MGARASVLSGGKLDQWEKIRLRPGGKKKYKLKHIVWASRELERFAVNPGLLETSEGCRQILRHLQPSLQTGSEELKSLYNTVATLYCVHQRMDVKDTKEALDKIEEEQNKSKKQAQQTAADAGNSSQSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYPISILDIRQGPKEPFRDYVDRFYKTLRAEQASQDVKNWMTDTLLVQNANPDCKTILKALGPGATLEEMMTACQGVGGPGHKARVLAEAMSQATNSANIMMQRGNFRNQRRTVKCFNCGKEGHIAKNCRAPRKKGCWKCGKEGHQMKDCTRQANFLGKIWSSHKGRPGNFLQNRPEPTAPPEESFRFGEETTTPSQKQGTIDKELYPLASLKSLFGNDPSSQ

SEQ ID NO: 46 - protein, 997 aa, Homo sapiens
FFREDLVFPQGEAREFPPEQTRANSPTRRELQVWGRDNNSLSEAGDDRQGTVSFSFPQITLWQRPLVTIKIGGQLKEALLDTGADDTVLEEMNLPGRWKPKMIGGIGGFIKVRQYDQVSIEICGHKTGTVLIGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPSGLKKKKSVTVLDVGDAYFSVPLDDFRKYTAFTIPSINNETPGIRYQYNVLPQGWKGSPAIFQSSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYGIKVKQLCKLLRGTKTLTEVVPLTEEAELELAENREILKEPVHGVYYDPSKDLIAEIQKQGQGQWTYQIYQEPFKNLKTGKYAKTRGAHTNDVKQLAEAVQKITTESIVIWGKTPRFKLPIQKETWEIWWTEYWQATWIPEWEFVNTPPVKLWYQLEKEPIVGAETFYVDGAANRETKLGKAGYVTDRGRQKVVSLTDTTNQKTELQAIHLALQDSGLEVNIVTDSQYALGIIQAQPDKSESELVSQIIEQLIKKEKVYLAWVPAHKGIGGNEQVDKLVSTGIRRVLFLDGIDKAQEEEKYHSNWRAMASDFNLPPIVAKEIVASCDKCQLKGEAMHGQVDCSPGIWQLDCTHLEGKVILVAVHVASGYIEAEVIPAETGQETAYFLLKLAGRWPVKTIHTDNGSNFTSTTVKAACWWAGIKQEFGIPYNPQSQGVVESMNKELKKIGQVRDQAEHLKTAVQMAVFIHNFKRKGGIGGYSAGERIIDIIATDIQTKELQKQITKIQNFRVYFRDSRDPLWKGPAKLLWKGEGAVVIQDNSDIKVVPRRKVKIIRDYGKQMAGDDCVASRQDED

SEQ ID NO: 47 - protein, 191 aa, Homo sapiens
MENRWQVMIVWQVDRMRIRTWKSLVKHHMYRSKKANGWFYRHHYESPHPRISSEVHIPLGDAKLVVTTYWGLHTGERDWHLGQGVSIEWRKKRYSTQVDPSLADQLIHLYYFDCFSESAIRNAILGHVSPRCEYQAGHNKVGSLQYLALAALITPKKIKPPLPSVKKLTEDRWNKPQKTKGYRGSHTMSGH

SEQ ID NO: 48 - protein, 96 aa, Homo sapiens
MEQAPENQGLQREPYNEWTLELLEELKSEAVRHFPQPWLHNLGQYIYETYGDTWTGVEAIIRILQQMLFIHFRIGCHHSRIGILRQRRTRNGASRS

SEQ ID NO: 49 - protein, 102 aa, Homo sapiens
MEPVDPRLEPWKHPGSQPSTPCTTCYCKICCFHCQVCFTTKGLGISYGRKKRRQRRRAPQDSQTHQVPLPEQPNSQPRGDPTGPKEPKEKVETKTEADPLDQ

SEQ ID NO: 50 - protein, 117 aa, Homo sapiens
MAGRSGDSDEELLRTVRLIKYLYQSDPIPSPEGTRQARRNRRRRWRQRQRQIHSLSEWILSTHLGRPTEPVPLQLPPLERLTLDCSEDCGTSGTQGVGSTQVSVESPTVLEPGTKEE

SEQ ID NO: 51 - protein, 81 aa, Homo sapiens
MQSLQILAIVALVVAVIIAIVVWTIVFIEYRKILRQRKIDRLIDRIRERAEDSGNESEGDQEELSALVEMGHDAPWDVDDL

SEQ ID NO: 52 - protein, 205 aa, Homo sapiens
MGGKWSKSSVVEWPAVRERMKRAEPAADGVGAVSRDLEKHGAITSSNTASNNADCAWLEAQEDEEVGFPVKPQVPLRPMTYKAAVDLSHFLKEKGGLEGLVYSQKRKDILDLWVYHTQGFFPDWQNYPGPGVRYPLTFGWCFKLVPLEQEQVEEANTGENRSLLHPMSLHGMDDPEKEVLVWKFDSRLAFHHVAREMHPEYYKDC

SEQ ID NO: 53 - protein, 9 aa, Homo sapiens
RLRPGGKKK

SEQ ID NO: 54 - protein, 9 aa, Homo sapiens
GSEELRSLY

SEQ ID NO: 55 - protein, 9 aa, Homo sapiens
ISPRTLNAW

SEQ ID NO: 56 - protein, 19 aa, Homo sapiens
YKTLRAEQATQEVKNWMTE

SEQ ID NO: 57 - protein, 19 aa, Homo sapiens
LLYQSDPPPSPEGTRQARR

SEQ ID NO: 58 - protein, 10 aa, Homo sapiens
RQDILDLWVY

SEQ ID NO: 59 - protein, 10 aa, Homo sapiens
HTQGYFPDWQ

SEQ ID NO: 60 - protein, 11 aa, Homo sapiens
ACQGVGGPGHK

SEQ ID NO: 61 - protein, 8 aa, Homo sapiens
CCFHCQVC

SEQ ID NO: 62 - protein, 11 aa, Homo sapiens
QEEHEKYHSNW

SEQ ID NO: 63 - protein, 19 aa, Homo sapiens
WNKPQKTKGYRGSHTMSGH

SEQ ID NO: 64 - protein, 19 aa, Homo sapiens
YKTLRAEQATQEVKNWMTE

SEQ ID NO: 65 - protein, 19 aa, Homo sapiens
NCGKEGHIAKNCRAPRKKG

SEQ ID NO: 66 - protein, 19 aa, Homo sapiens
APQDSQTHQASISKQPTSQ

SEQ ID NO: 67 - protein, 19 aa, Homo sapiens
RQRKLDRLIERIRERAEDS

SEQ ID NO: 68 - protein, 17 aa, Homo sapiens
ESDGDQEELLVEMGHLA

SEQ ID NO: 69 - protein, 19 aa, Homo sapiens
FKLVPVDPDEVEEANEGEN

SEQ ID NO: 70 - protein, 19 aa, Homo sapiens
LHGLGQQIYGTYGDTWEGV

SEQ ID NO: 71 - protein, 19 aa, Homo sapiens
SQTHQVPLPEQPNSQPRGD

SEQ ID NO: 72 - protein, 19 aa, Homo sapiens
EDEEVGFPVKPQVPLRPMT

SEQ ID NO: 73 - protein, 10 aa, Homo sapiens
TSTLQEQIGW

SEQ ID NO: 74 - protein, 10 aa, Homo sapiens
AISPRTLNAW

SEQ ID NO: 75 - protein, 10 aa, Homo sapiens
STTVKAACWW

SEQ ID NO: 76 - protein, 9 aa, Homo sapiens
IVLPEKDSW

SEQ ID NO: 77 - protein, 9 aa, Homo sapiens
QASQEVKNW

SEQ ID NO: 78 - protein, 11 aa, Homo sapiens
KAFSPEVIPMF

SEQ ID NO: 79 - protein, 9 aa, Homo sapiens
KTAVQMAVF

SEQ ID NO: 80 - protein, 9 aa, Homo sapiens
FPRIWLHGL

SEQ ID NO: 81 - protein, 12 aa, Homo sapiens
APQGHPNNQVSI

SEQ ID NO: 82 - protein, 17 aa, Homo sapiens
AALVEMGHDAPWVVDDL

SEQ ID NO: 83 - protein, 10 aa, Homo sapiens
QIRSLSGWIL

SEQ ID NO: 84 - protein, 19 aa, Homo sapiens
LHGLGQQIYGTYGDTWEGV

SEQ ID NO: 85 - protein, 17 aa, Homo sapiens
MGGKWSKSSLIGWPRIR

SEQ ID NO: 86 - protein, 19 aa, Homo sapiens
EDVERANEGGNNCLLHPMS

SEQ ID NO: 87 - protein, 11 aa, Homo sapiens
ACQGVGGPGHK

SEQ ID NO: 88 - protein, 9 aa, Homo sapiens
FSFPQITLW

SEQ ID NO: 89 - protein, 9 aa, Homo sapiens
ALIRILQQL

SEQ ID NO: 90 - protein, 9 aa, Homo sapiens
ALIRILQQL

SEQ ID NO: 91 - protein, 8 aa, Homo sapiens
CCFHCQVC

SEQ ID NO: 92 - protein, 9 aa, Homo sapiens
SAEPVPLQL

SEQ ID NO: 93 - protein, 10 aa, Homo sapiens
QVLGESPTVL

SEQ ID NO: 94 - protein, 10 aa, Homo sapiens
QVLGESPTVL

SEQ ID NO: 95 - protein, 19 aa, Homo sapiens
QNKSKKKAQQAAADTGNNS

SEQ ID NO: 96 - protein, 19 aa, Homo sapiens
NFLQSRPEPTAPTAPTAPP

SEQ ID NO: 97 - protein, 19 aa, Homo sapiens
EICGHKAIGAVLVGPTPVN

SEQ ID NO: 98 - protein, 19 aa, Homo sapiens
TEMEKEGKISKIGPENPYN

SEQ ID NO: 99 - protein, 13 aa, Homo sapiens
MQSLETIAIVALV

SEQ ID NO: 100 - protein, 19 aa, Homo sapiens
AACTWLEAQEQEEEVGFPV

SEQ ID NO: 101 - protein, 10 aa, Homo sapiens
TNSANIMMQR

SEQ ID NO: 102 - protein, 10 aa, Homo sapiens
TNSANIMMQR

SEQ ID NO: 103 - protein, 17 aa, Homo sapiens
QKQGTIDKELYPLASLK

SEQ ID NO: 104 - protein, 17 aa, Homo sapiens
QKQGTIDKELYPLASLK

SEQ ID NO: 105 - protein, 16 aa, Homo sapiens
QVDKLVSTGIRRVLFL

SEQ ID NO: 106 - protein, 11 aa, Homo sapiens
QEEHEKYHSNW

SEQ ID NO: 107 - protein, 9 aa, Homo sapiens
HIVSPRCEY

SEQ ID NO: 108 - protein, 18 aa, Homo sapiens
MEQAPENQGLQREPYNEW

SEQ ID NO: 109 - protein, 9 aa, Homo sapiens
SEWILSTHL

SEQ ID NO: 110 - protein, 19 aa, Homo sapiens
GSTQVSVESPTVLEPGTKE

SEQ ID NO: 111 - protein, 10 aa, Homo sapiens
VEWPAVRERM

SEQ ID NO: 112 - protein, 10 aa, Homo sapiens
KAAVDLSHFL

SEQ ID NO: 113 - protein, 18 aa, Homo sapiens
SHFLKEKFFLEFLVYSQK

SEQ ID NO: 114 - protein, 9 aa, Homo sapiens
DSRLAFHHV

SEQ ID NO: 115 - protein, 19 aa, Homo sapiens
EGCRQILRHLQPSLQTGSE

SEQ ID NO: 116 - protein, 19 aa, Homo sapiens
EGCRQILRHLQPSLQTGSE

SEQ ID NO: 117 - protein, 19 aa, Homo sapiens
ELHPDKWTVQPIVLPEKDS

SEQ ID NO: 118 - protein, 19 aa, Homo sapiens
VGSLQYLALAALITPKKIK

SEQ ID NO: 119 - protein, 19 aa, Homo sapiens
SQTHQVPLPEQPNSQPRGD

SEQ ID NO: 120 - protein, 19 aa, Homo sapiens
VALVVAVIIAIVVWTIVFI

SEQ ID NO: 121 - protein, 13 aa, Homo sapiens
EMGHDAPWDVDDL

SEQ ID NO: 122 - protein, 19 aa, Homo sapiens
RGDPTGPKEPKEKVETKTE

SEQ ID NO: 123 - protein, 19 aa, Homo sapiens
EDEEVGFPVKPQVPLRPMT

SEQ ID NO: 124 - protein, 19 aa, Homo sapiens
VEEANTGENRSLLHPMSLH

SEQ ID NO: 125 - protein, 9 aa, Homo sapiens
RLRPGGKKK

SEQ ID NO: 126 - protein, 9 aa, Homo sapiens
GSEELRSLY

SEQ ID NO: 127 - protein, 9 aa, Homo sapiens
ISPRTLNAW

SEQ ID NO: 128 - protein, 10 aa, Homo sapiens
TSTLQEQIGW

SEQ ID NO: 129 - protein, 20 aa, Homo sapiens
KEMYPLASLRSLFGNDPSSQ

SEQ ID NO: 130 - protein, 9 aa, Homo sapiens
AIFQSSMTK

SEQ ID NO: 131 - protein, 9 aa, Homo sapiens
IVLPEKDSW

SEQ ID NO: 132 - protein, 9 aa, Homo sapiens
QIYVGIKVR

SEQ ID NO: 133 - protein, 9 aa, Homo sapiens
QIYAGIKVK

SEQ ID NO: 134 - protein, 10 aa, Homo sapiens
STTVKAACWW

SEQ ID NO: 135 - protein, 10 aa, Homo sapiens
STTVKAACWW

SEQ ID NO: 136 - protein, 10 aa, Homo sapiens
RIRTWKSLVK

SEQ ID NO: 137 - protein, 9 aa, Homo sapiens
VSKKAKGWF

SEQ ID NO: 138 - protein, 10 aa, Homo sapiens
HPRISSEVHI

SEQ ID NO: 139 - protein, 11 aa, Homo sapiens
RIKPPLPSVTK

SEQ ID NO: 140 - protein, 11 aa, Homo sapiens
RIKPPLPSVTK

SEQ ID NO: 141 - protein, 10 aa, Homo sapiens
TLELLEELKN

SEQ ID NO: 142 - protein, 10 aa, Homo sapiens
KTGRLIKLLY

SEQ ID NO: 143 - protein, 10 aa, Homo sapiens
KTGRLIKLLY

SEQ ID NO: 144 - protein, 16 aa, Homo sapiens
RSAEPAPLQLPPLERL

SEQ ID NO: 145 - protein, 9 aa, Homo sapiens
RPMTYKGAL

SEQ ID NO: 146 - protein, 10 aa, Homo sapiens
RQDILDLWVY

SEQ ID NO: 147 - protein, 10 aa, Homo sapiens
HTQGYFPDWQ

SEQ ID NO: 148 - protein, 9 aa, Homo sapiens
YFPDWQDYT

SEQ ID NO: 149 - protein, 17 aa, Homo sapiens
AFRHMARELHPEYYKNC

SEQ ID NO: 150 - protein, 19 aa, Homo sapiens
YKTLRAEQATQEVKNWMTE

SEQ ID NO: 151 - protein, 19 aa, Homo sapiens
NCGKEGHIAKNCRAPRKKG

SEQ ID NO: 152 - protein, 19 aa, Homo sapiens
NLPGRWKPKMIGGIGGFIK

SEQ ID NO: 153 - protein, 19 aa, Homo sapiens
LDCTHLEGKIILVAVHVAS

SEQ ID NO: 154 - protein, 19 aa, Homo sapiens
MNKELKKIIGQVRDQAEHL

SEQ ID NO: 155 - protein, 19 aa, Homo sapiens
KFKRKGGIGEYSAGERIVD

SEQ ID NO: 156 - protein, 19 aa, Homo sapiens
GEYSAGERIIDIIATDIQT

SEQ ID NO: 157 - protein, 19 aa, Homo sapiens
TYWGLHTGERDWHLGQGVS

SEQ ID NO: 158 - protein, 19 aa, Homo sapiens
CFHCQVCFITKGLGISYGR

SEQ ID NO: 159 - protein, 19 aa, Homo sapiens
APQDSQTHQASISKQPTSQ

SEQ ID NO: 160 - protein, 11 aa, Homo sapiens
MHSLTIVAIVA

SEQ ID NO: 161 - protein, 19 aa, Homo sapiens
YRKILRQRKLDRLIERIRE

SEQ ID NO: 162 - protein, 19 aa, Homo sapiens
RQRKLDRLIERIRERAEDS

SEQ ID NO: 163 - protein, 17 aa, Homo sapiens
ESDGDQEELLVEMGHLA

SEQ ID NO: 164 - protein, 17 aa, Homo sapiens
ESDGDQEELLVEMGHLA

SEQ ID NO: 165 - protein, 18 aa, Homo sapiens
EELLVEMGHLAPWDINDL

SEQ ID NO: 166 - protein, 12 aa, Homo sapiens
MGHLAPWDINDL

SEQ ID NO: 167 - protein, 19 aa, Homo sapiens
LLYQSDPPPSPEGTRQARR

SEQ ID NO: 168 - protein, 12 aa, Homo sapiens
VERETETHPVDQ

SEQ ID NO: 169 - protein, 19 aa, Homo sapiens
ERLTLDCSEGCGTSGTQGV

SEQ ID NO: 170 - protein, 19 aa, Homo sapiens
GTSGTQGVGGPQILVESPA

SEQ ID NO: 171 - protein, 17 aa, Homo sapiens
MGGKWSKGIGRGWPAVR

SEQ ID NO: 172 - protein, 19 aa, Homo sapiens
GGKWSKGIGRGWPAVRERM

SEQ ID NO: 173 - protein, 19 aa, Homo sapiens
RRAAPADGVGTASQRAEPA

SEQ ID NO: 174 - protein, 19 aa, Homo sapiens
GAITSSNLAANNADVAWLE

SEQ ID NO: 175 - protein, 18 aa, Homo sapiens
ADVAWLEAQEEEEVGFPV

SEQ ID NO: 176 - protein, 19 aa, Homo sapiens
FKLVPVDPDEVEEANEGEN

SEQ ID NO: 177 - protein, 9 aa, Homo sapiens
KIRLRPGGK

SEQ ID NO: 178 - protein, 9 aa, Homo sapiens
GPGHKARVL

SEQ ID NO: 179 - protein, 9 aa, Homo sapiens
HPVHAGPIA

SEQ ID NO: 180 - protein, 11 aa, Homo sapiens
KAFSPEVIPMF

SEQ ID NO: 181 - protein, 11 aa, Homo sapiens
RMRGAHTNDVK

SEQ ID NO: 182 - protein, 9 aa, Homo sapiens
SPAIFQSSM

SEQ ID NO: 183 - protein, 9 aa, Homo sapiens
KTAVQMAVF

SEQ ID NO: 184 - protein, 9 aa, Homo sapiens
FPRIWLHGL

SEQ ID NO: 185 - protein, 9 aa, Homo sapiens
AVRHFPRIW

SEQ ID NO: 186 - protein, 10 aa, Homo sapiens
WRFDSRLAFH

SEQ ID NO: 187 - protein, 9 aa, Homo sapiens
TPQVPLRPM

SEQ ID NO: 188 - protein, 20 aa, Homo sapiens
DFRKYTAFTIPSINNETPGI

SEQ ID NO: 189 - protein, 18 aa, Homo sapiens
GQGQWTYQIYQEPFKNLK

SEQ ID NO: 190 - protein, 18 aa, Homo sapiens
QKIATESIVIWGKTPKFK

SEQ ID NO: 191 - protein, 17 aa, Homo sapiens
NFTSAAVKAACWWANVQ

SEQ ID NO: 192 - protein, 18 aa, Homo sapiens
TKELQKQITKIQNFRVYY

SEQ ID NO: 193 - protein, 17 aa, Homo sapiens
TVQPIVLPEKDSWTVND

SEQ ID NO: 194 - protein, 18 aa, Homo sapiens
MEQAPEDQGPQREPYNEW

SEQ ID NO: 195 - protein, 17 aa, Homo sapiens
SLGQHIYETYGDTWAGV

SEQ ID NO: 196 - protein, 17 aa, Homo sapiens
ETYGDTWAGVEAIIRIL

SEQ ID NO: 197 - protein, 18 aa, Homo sapiens
AGVEAIIRILQQLLFIHF

SEQ ID NO: 198 - protein, 18 aa, Homo sapiens
IVALVVAAIIAIVVWSIV

SEQ ID NO: 199 - protein, 11 aa, Homo sapiens
AEQASQDVKNW

SEQ ID NO: 200 - protein, 9 aa, Homo sapiens
AIFQSSMTK

SEQ ID NO: 201 - protein, 9 aa, Homo sapiens
QIYAGIKVK

SEQ ID NO: 202 - protein, 9 aa, Homo sapiens
QIIEQLIKK

SEQ ID NO: 203 - protein, 10 aa, Homo sapiens
IYQEPFKNLK

SEQ ID NO: 204 - protein, 10 aa, Homo sapiens
LADQLIHLHY

SEQ ID NO: 205 - protein, 10 aa, Homo sapiens
LTFGWCFKLV

SEQ ID NO: 206 - protein, 10 aa, Homo sapiens
QVPLRPMTYK

SEQ ID NO: 207 - protein, 9 aa, Homo sapiens
AVDLSHFLK

SEQ ID NO: 208 - protein, 9 aa, Homo sapiens
YPLTFGWCY

SEQ ID NO: 209 - protein, 19 aa, Homo sapiens
KIEELRQHLLRWGFTTPDK

SEQ ID NO: 210 - protein, 18 aa, Homo sapiens
GQGQWTYQIYQEPFKNLK

SEQ ID NO: 211 - protein, 18 aa, Homo sapiens
GIWQLDCTHLEGKIILVA

SEQ ID NO: 212 - protein, 18 aa, Homo sapiens
DDTVLEEMNLPGRWKPKM

SEQ ID NO: 213 - protein, 17 aa, Homo sapiens
LSKQPASQPRGDPTGPK

SEQ ID NO: 214 - protein, 15 aa, Homo sapiens
TYKAAVDLSHFLKEK

SEQ ID NO: 215 - protein, 9 aa, Homo sapiens
DRFYKTLRA

SEQ ID NO: 216 - protein, 11 aa, Homo sapiens
AEQASQDVKNW

SEQ ID NO: 217 - protein, 9 aa, Homo sapiens
CRAPRKKGC

SEQ ID NO: 218 - protein, 9 aa, Homo sapiens
ITLWQRPLV

SEQ ID NO: 219 - protein, 9 aa, Homo sapiens
DTVLEEMNL

SEQ ID NO: 220 - protein, 15 aa, Homo sapiens
PIVQNLQGQMVHQAI

SEQ ID NO: 221 - protein, 16 aa, Homo sapiens
FSALSEGATPQDLNTM

SEQ ID NO: 222 - protein, 19 aa, Homo sapiens
KIEELRQHLLRWGFTTPDK

SEQ ID NO: 223 - protein, 20 aa, Homo sapiens
YYDPSKDLIAEIQKQGQGQW

SEQ ID NO: 224 - protein, 17 aa, Homo sapiens
GAETFYVDGAANRETKL

SEQ ID NO: 225 - protein, 17 aa, Homo sapiens
QDSGLEVNIVTDSQYAL

SEQ ID NO: 226 - protein, 16 aa, Homo sapiens
DGAANRETKLGKAGYV

SEQ ID NO: 227 - protein, 15 aa, Homo sapiens
HHMYISKKAKGWFYR

SEQ ID NO: 228 - protein, 18 aa, Homo sapiens
EWTLELLEELKNEAVRHF

SEQ ID NO: 229 - protein, 18 aa, Homo sapiens
MAGRSGDSDEELLKTVRL

SEQ ID NO: 230 - protein, 15 aa, Homo sapiens
FDSRLAFHHVARELH